SEQUENCE LISTING (555 sequences)

SEQ ID NO: 1-2 - skipped (2 entries)

SEQ ID NO: 3 - RNA, 10 nt, synthetic construct
  source 1..10: mol_type = other RNA; note = Description of Artificial Sequence: Synthetic oligonucleotide
tttttttttt

SEQ ID NO: 4 - DNA, 10 bp, synthetic construct
  source 1..10: mol_type = other DNA; note = Description of Artificial Sequence: Synthetic oligonucleotide; note = Description of Combined DNA/RNA Molecule: Synthetic oligonucleotide
  misc_feature 1..4: note = DNA
  misc_feature 5..6: note = RNA
  misc_feature 7..10: note = DNA
tttttttttt

SEQ ID NO: 5-11 - skipped (7 entries)

SEQ ID NO: 12 - DNA, 10 bp, synthetic construct
  source 1..10: mol_type = other DNA; note = Description of Artificial Sequence: Synthetic oligonucleotide
tttttttttt

SEQ ID NO: 13 - DNA, 12 bp, synthetic construct
  source 1..12: mol_type = other DNA; note = Description of Artificial Sequence: Synthetic oligonucleotide
tttttttttttt

SEQ ID NO: 14 - DNA, 14 bp, synthetic construct
  source 1..14: mol_type = other DNA; note = Description of Artificial Sequence: Synthetic oligonucleotide
tttttttttttttt

SEQ ID NO: 15-17 - skipped (3 entries)

SEQ ID NO: 18 - protein, 1228 aa, unidentified
  source 1..1228: note = Description of Unknown: Lachnospiraceae bacterium sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 19 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 20 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 21 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 22 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 23 - protein, 1259 aa, Moraxella bovoculi
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 24 - protein, 1269 aa, Moraxella bovoculi
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 25 - protein, 1306 aa, Thiomicrospira sp.
MGIHGVPAATKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 26 - protein, 1214 aa, Butyrivibrio sp.
MGIHGVPAAYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 27 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 28 - protein, 1224 aa, synthetic construct
  source 1..1224: note = Description of Artificial Sequence: Synthetic polypeptide
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 29 - protein, 1297 aa, synthetic construct
  source 1..1297: note = Description of Artificial Sequence: Synthetic polypeptide
MATLVSFTKQYQVQKTLRFELIPQGKTQANIDAKGFINDDLKRDENYMKVKGVIDELHKNFIEQTLVNVDYDWRSLATAIKNYRKDRSDTNKKNLEKTQEAARKEIIAWFEGKRGNSAFKNNQKSFYGKLFKKELFSEILRSDDLEYDEETQDAIACFDKFTTYFVGFHENRKNMYSTEAKSTSVAYRVVNENFSKFLSNCEAFSVLEAVCPNVLVEAEQELHLHKAFSDLKLSDVFKVEAYNKYLSQTGIDYYNQIIGGISSAEGVRKIRGVNEVVNNAIQQNDELKVALRNKQFTMVQLFKQILSDRSTLSFVSEQFTSDQEVITVVKQFNDDIVNNKVLAVVKTLFENFNSYDLEKIYINSKELASVSNALLKDWSKIRNAVLENKIIELGANPPKTKISAVEKEVKNKDFSIAELASYNDKYLDKEGNDKEICSIANVVLEAVGALEIMLAESLPADLKTLENKNKVKGILDAYENLLHLLNYFKVSAVNDVDLAFYGAFEKVYVDISGVMPLYNKVRNYATKKPYSVEKFKLNFAMPTLADGWDKNKERDNGSIILLKDGQYYLGVMNPQNKPVIDNAVCNDAKGYQKMVYKMFPEISKMVTKCSTQLNAVKAHFEDNTNDFVLDDTDKFISDLTITKEIYDLNNVLYDGKKKFQIDYLRNTGDFAGYHKALETWIDFVKEFLSKYRSTAIYDLTTLLPTNYYEKLDVFYSDVNNLCYKIDYENISVEQVNEWVEEGNLYLFKIYNKDFATGSTGKPNLHTMYWNAVFAEENLHDVVVKLNGGAELFYRPKSNMPKVEHRVGEKLVNRKNVNGEPIADSVHKEIYAYANGKISKSELSENAQEELPLAIIKDVKHNITKDKRYLSDKYFFHVPITLNYKANGNPSAFNTKVQAFLKNNPDVNIIGIDRGERNLLYVVVIDQQGNIIDKKQVSYNKVNGYDYYEKLNQREKERIEARQSWGAVGKIKELKEGYLSLVVREIADMMVKYNAIVVMENLNAGFKRVRGGIAEKAVYQKFEKMLIDKLNYLVFKDVEAKEAGGVLNAYQLTDKFDSFEKMGNQSGFLFYVPAAYTSKIDPVTGFANVFSTKHITNTEAKKEFICSFNSLRYDEAKDKFVLECDLNKFKIVANSHIKNWKFIIGGKRIVYNSKNKTYMEKYPCEDLKATLNASGIDFSSSEIINLLKNVPANREYGKLFDETYWAIMNTLQMRNSNALTGEDYIISAVADDNEKVFDSRTCGAELPKDADANGAYHIALKGLYLLQRIDISEEGEKVDLSIKNEEWFKFVQQKEYAR

SEQ ID NO: 30 - protein, 1229 aa, synthetic construct
  source 1..1229: note = Description of Artificial Sequence: Synthetic polypeptide
MKEQFINRYPLSKTLRFSLIPVGETENNFNKNLLLKKDKQRAENYEKVKCYIDRFHKEYIESVLSKARIEKVNEYANLYWKSNKDDSDIKAMESLENDMRKQISKQLTSTEIYKKRLFGKELICEDLPSFLTDKDERETVECFRSFTTYFKGFNTNRENMYSSDGKSTAIAYRCINDNLPRFLDNVKSFQKVFDNLSDETITKLNTDLYNIFGRNIEDIFSVDYFEFVLTQSGIEIYNSMIGGYTCSDKTKIQGLNECINLYNQQVAKNEKSKKLPLMKPLYKQILSEKDSVSFIPEKFNSDNEVLHAIDDYYTGHIGDFDLLTELLQSLNTYNANGIFVKSGVAITDISNGAFNSWNVLRSAWNEKYEALHPVTSKTKIDKYIEKQDKIYKAIKSFSLFELQSLGNENGNEITDWYISSINESNSKIKEAYLQAQKLLNSDYEKSYNKRLYKNEKATELVKNLLDAIKEFQKLIKPLNGTGKEENKDELFYGKFTSYYDSIADIDRLYDKVRNYITQKPYSKDKIKLNFDNPQLLGGWDKNKESDYRTVLLHKDGLYYLAVMDKSHSKAFVDAPEITSDDKDYYEKMEYKLLPGPNKMLPKVFFASKNIDTFQPSDRILDIRKRESFKKGATFNKAECHEFIDYFKDSIKKHDDWSQFGFKFSPTESYNDISEFYREISDQGYSVRFNKISKNYIDGLVNNGYIYLFQIYNKDFSKYSKGTPNLHTLYFKMLFDERNLSNVVYKLNGEAEMFYREASIGDKEKITHYANQPIKNKNPDNEKKESVFEYDIVKDKRFTKRQFSLHLPITINFKAHGQEFLNYDVRKAVKYKDDNYVIGIDRGERNLIYISVINSNGEIVEQMSLNEIISDNGHKVDYQKLLDTKEKERDKARKNWTSVENIKELKEGYISQVVHKICELVIKYDAVIAMEDLNFGFKRGRFPVEKQVYQKFENMLISKLNLLIDKKAEPTEDGGLLRAYQLTNKFDGVNKAKQNGIIFYVPAWDTSKIDPATGFVNLLKPKCNTSVPEAKKLFETIDDIKYNANTDMFEFYIDYSKFPRCNSDFKKSWTVCTNSSRILTFRNKEKNNKWDNKQIVLTDEFKSLFNEFGIDYKGNLKDSILSISNADFYRRLIKLLSLTLQMRNSITGSTLPEDDYLISPVANKSGEFYDSRNYKGTNAALPCDADANGAYNIARKALWAINVLKDTPDDMLNKAKLSITNAEWLEYTQK

SEQ ID NO: 31 - protein, 1470 aa, synthetic construct
  source 1..1470: note = Description of Artificial Sequence: Synthetic polypeptide
MNNPRGAFGGFTNLYSLSKTLRFELKPYLEIPEGEKGKLFGDDKEYYKNCKTYTEYYLKKANKEYYDNEKVKNTDLQLVNFLHDERIEDAYQVLKPVFDTLHEEFITDSLESAEAKKIDFGNYYGLYEKQKSEQNKDEKKKIDKPLETERGKLRKAFTPIYEAEGKNLKNKAGKEKKDKDILKESGFKVLIEAGILKYIKNNIDEFADKKLKNNEGKEITKKDIETALGAENIEGIFDGFFTYFSGFNQNRENYYSTEEKATAVASRIVDENLSKFCDNILLYRKNENDYLKIFNFLKNKGKDLKLKNSKFGKENEPEFIPAYDMKNDEKSFSVADFVNCLSQGEIEKYNAKIANANYLINLYNQNKDGNSSKLSMFKILYKQIGCGEKKDFIKTIKDNAELKQILEKACEAGKKYFIRGKSEDGGVSNIFDFTDYIQSHENYKGVYWSDKAINTISGKYFANWDTLKNKLGDAKVFNKNTGEDKADVKYKVPQAVMLSELFAVLDDNAGEDWREKGIFFKASLFEGDQNKSEIIKNANRPSQALLKMICDDMESLAKNFIDSGDKILKISDRDYQKDENKQKIKNWLDNALWINQILKYFKVKANKIKGDSIDARIDSGLDMLVFSSDNPAEDYDMIRNYLTQKPQDEINKLKLNFENSSLAGGWDENKEKDNSCIILKDEQDKQYLAVMKYENTKVFEQKNSQLYIADNAAWKKMIYKLVPGASKTLPKVFFSKKWTANRPTPSDIVEIYQKGSFKKENVDFNDKKEKDESRKEKNREKIIAELQKTCWMDIRYNIDGKIESAKYVNKEKLAKLIDFYKENLKKYPSEEESWDRLFAFGFSDTKSYKSIDQFYIEVDKQGYKLEFVTINKARLDEYVRDGKIYLFEIRSRDNNLVNGEEKTSAKNLQTIYWNAAFGGDDNKPKLNGEAEIFYRPAIAENKLNKKKDKNGKEIIDGYRFSKEKFIFHCPITLNFCLKETKINDKLNAALAKPENGQGVYFLGIDRGEKHLAYYSLVNQKGEILEQGTLNLPFLDKNGKSRSIKVEKKSFEKDSNGGIIKDKDGNDKIKIEFVECWNYNDLLDARAGDRDYARKNWTTIGTIKELKDGYISQVVRKIVDLSIYKNTETKEFREMPAFIVLEDLNIGFKRGRQKIEKQVYQKLELALAKKLNFLVDKKADIGEIGSVTKAIQLTPPVNNFGDMENRKQFGNMLYIRADYTSQTDPATGWRKSIYLKSGSESNVKEQIEKSFFDIRYESGDYCFEYRDRHGKMWQLYSSKNGVSLDRFHGERNNSKNVWESEKQPLNEMLDILFDEKRFDKSKSLYEQMFKGGVALTRLPKEINKKDKPAWESLRFVIILIQQIRNTGKNGDDRNGDFIQSPVRDEKTGEHFDSRIYLDKEQKGEKADLPTSGDANGAYNIARKGIVVAEHIKRGFDKLYISDEEWDTWLAGDEIWDKWLKENRESLTKTRK

SEQ ID NO: 32 - protein, 1282 aa, synthetic construct
  source 1..1282: note = Description of Artificial Sequence: Synthetic polypeptide
MNGNRIIVYREFVGVTPVAKTLRNELRPIGHTQEHIIHNGLIQEDELRQEKSTELKNIMDDYYREYIDKSLSGVTDLDFTLLFELMNLVQSSPSKDNKKALEKEQSKMREQICTHMQSDSNYKNIFNAKFLKEILPDFIKNYNQYDAKDKAGKLETLALFNGFSTYFTDFFEKRKNVFTKEAVSTSIAYRIVHENSLTFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKENDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMSKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 33 - protein, 1225 aa, synthetic construct
  source 1..1225: note = Description of Artificial Sequence: Synthetic polypeptide
MKKIDSFVNYYPLSKTLRFSLIPVGKTEDNFNAKLLLEEDEKRAIEYEKVKRYIDRYHKHFIETVLANFHLDDVNEYAELYYKAGKDDKDLKYMEKLEGKMRKSISAAFTKDKKYKEIFGQEIIKNILPEFLENEDEKESVKMFQGFFTYFTGFNDNRKNMYTHEAQTTAISYRCINENLPKFLDNVQSFAKIKESISSDIMNKLDEVCMDLYGVYAQDMFCTDYFSFVLSQSGIDRYNNIIGGYVDDKGVKIQGINEYINLYNQQVDEKNKRLPLMKKLYKQILIEKESISFIPEKFESDNIVINAISDYYHNNVENLFDDFNKLFNEFSEYDDNGIFVTSGLAVTDISNAVFGSWNIISDSWNEEYKDSHPMKKTTNAEKYYEDMKKEYKKNLSFTIAELQRLGEAGCNDECKGDIKEYYKTTVAEKIENIKNAYEISKDLLASDYEKSNDKKLCKNDSAISLLKNLLDSIKDLEKTIKPLLGTGKEENKDDVFYGKFTNLYEMISEIDRLYDKVRNYVTQKPYSKDKIKLNFENPQHLGGWDKNKERDYRSVLLKKEDKYYLAIMDKSNNKAFIDFPDDGECYEKIEYKLLPGPNKMLPKVFFASSNIEYFAPSKKILEIRSRESFKKGDMFNLKDCHEFIDFFKESIKKHEDWSQFGFEFSPTEKYNDISEFYNEVKIQGYSLKYKNVSKKYIDELIECGQLYLFQIYNKDFSVYAKGNPNLHTMYFKMLFDERNLANVVYQLNGGAEMFYRKASIKDSEKIVHHANQPIKNKNADNVKKESVFEYDIIKDKRFTKRQFSIHIPITLNFKAKGQNFINNDVRMALKKADENYVIGIDRGERNLLYICVINSKGEIVEQKSLNEIIGDNGYRVDYHKLLDKKEAERDEARKSWGTIENIKELKEGYLSQIVHEISKLVIKYDAVIAIEDLNSGFKKGRFKVEKQVYQKFENMLCTKLNYLVDKNADANECGGLLKAYQLTNKEDGANRGRQNGIIFSVPAWLTSKIDPVTGFADLLRPKYKSVSESVEFISKIDNIRYNSKEDYFEFDIDYSKFPNSTASYKKKWTVCTYGERIINVRNKEKNNMWDNKTIVLTDEFKKLFADFGVDVSKNIKESVLAIDSKDFYYRFINLLANTLQLRNSEVGNVDVDYLISPVKGVDGSFYDSRLVKEKTLPENADANGAYNIARKALWAIDVLKQTKDEELKNANLSIKNAEWLEYVQK

SEQ ID NO: 34 - protein, 1293 aa, synthetic construct
  source 1..1293: note = Description of Artificial Sequence: Synthetic polypeptide
MRTMVTFEDFTKQYQVSKTLRFELIPQGKTLENMKRDGIISVDRQRNEDYQKAKGILDKLYKYILDFTMETVVIDWEALATATEEFRKSKDKKTYEKVQSKIRTALLEHVKKQKVGTEDLFKGMFSSKIITGEVLAAFPEIRLSDEENLILEKFKDFTTYFTGFFENRKNVFTDEALSTSFTYRLVNDNFIKFFDNCIVFKNVVNISPHMAKSLETCASDLGIFPGVSLEEVFSISFYNRLLTQTGIDQFNQLLGGISGKEGEHKQQGLNEIINLAMQQNLEVKEVLKNKAHRFTPLFKQILSDRSTMSFIPDAFADDDEVLSAVDAYRKYLSEKNIGDRAFQLISDMEAYSPELMRIGGKYVSVLSQLLFYSWSEIRDGVKAYKESLITGKKTKKELENIDKEIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAILLRKEGEYFLGIFNSKNKPKLVSDGGAGIGYEKMIYKQFPDFKKMLPKCTISLKDTKAHFQKSDEDFTLQTDKFEKSIVITKQIYDLGTQTVNGKKKFQVDYPRLTGDMEGYRAALKEWIDFGKEFIQAYTSTAIYDTSLFRDSSDYPDLPSFYKDVDNICYKLTFEWIPDAVIDDCIDDGSLYLFKLHNKDFSSGSIGKPNLHTLYWKALFEEENLSDVVVKLNGQAELFYRPKSLTRPVVHEEGEVIINKTTSTGLPVPDDVYVELSKFVRNGKKGNLTDKAKNWLDKVTVRKMPHAITKDRRFTVDKFFFHVPITLNYKADSSPYRFNDFVRQYIKDCSDVKIIGIDRGERNLIYAVVIDGKGNIIEQRSFNTVGTYNYQEKLEQKEKERQTARQDWATVTKIKDLKKGYLSAVVHELSKMIVKYKAIVALENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPYDADANGAFHIAMKGLLLTERIRNDDKLAISNEEWLNYIQEMRG

SEQ ID NO: 35 - protein, 1399 aa, synthetic construct
  source 1..1399: note = Description of Artificial Sequence: Synthetic polypeptide
MNKDIRKNFTDFVGISEIQKTLRFILIPIGKTAQNIDKYNMFEDDEIRHEYYPILKEACDDFYRNHIDQQFENLELDWSKLDEALASEDRDLINETRATYRQVLFNRLKNSVDIKGDSKKNKTLSLESSDKNLGKKKTKNTFQYNFNDLFKAKLIKAILPLYIEYIYEGEKLENAKKALKMYNRFTSRLSNFWQARANIFTDDEISTGSPYRLVNDNFTIFRINNSIYTKNKPFIEEDILEFEKKLKSKKIIKDFESVDDYFTVNAFNKLCTQNGIDKYNSILGGFTTKEREKVKGLNELFNLAQQSINKGKKGEYRKNIRLGKLTKLKKQILAISDSTSFLIEQIEDDQDLYNKIKDFFELLLKEEIENENIFTQYANLQKLIEQADLSKIYINAKHLNKISHQVTGKWDSLNKGIALLLENININEESKEKSEVISNGQTKDISSEAYKRYLQIQSEEKDIERLRTQIYFSLEDLEKALDLVLIDENMDRSDKSILSYVQSPDLNVNFERDLTDLYSRIMKLEENNEKLLANHSAIDLIKEFLDLIMLRYSRWQILFCDSNYELDQTFYPIYDAVMEILSNIIRLYNLARNYLSRKPDRMKKKKINFNNPTLADGWSESKIPDNSSMLFIKDGMYYLGIIKNRAAYSELLEAESLQSSEKKKSENSSYERMNYHFLPDAFRSIPKSSIAMKAVKEHFEINQKTADLLLDTDKFSKPLRITKEIFDMQYVDLHKNKKKYQVDYLRDTGDKKGYRKALNTWLNFCKDFISKYKGRNLFDYSKIKDADHYETVNEFYNDVDKYSYHIFFTSVAETTVEKFISEGKLYLFQLYNKDFSPHSTGKPNLHTIYWRALFSEENLTSKNIKLNGQAEIFFRPKQIETPFTHKKGSILVNRFDVNGNPIPINVYQEIKGFKNNVIKWDDLNKTTQEGLENDQYLYFESEFEIIKDRRYTEDQLFFHVPISFNWDIGSNPKINDLATQYIVNSNDIHIIGIDRGENHLIYYSVIDLQGAIVEQGSLNTITEYTENKFLNNKTNNLRKIPYKDILQQREDERADARIKWHAIDKIKDLKDGYLGQIVHFLAKLIIKYNAIVILEDLNYGFKRGRFKVERQVYQKFEMALMKKLNVLVFKDYDIDEIGGPLKPWQLTRPIDSYERMGRQNGILFYVPAAYTSAVDPVTGFANLFYLNNVKNSEKFHFFSKFESIKYHSDQDMFSFAFDYNNFGTTTRINDLSKSKWQVFTNHERSVWNNKEKNYVTQNLTDLIKKLLRTYNIEFKNNQNVLDSILKIENNTDKENFARELFRLFRLTIQLRNTTVNENNTEITENELDYIISPVKDKNGNFFDSRDELKNLPDNGDANGAYNIARKGLLYIEQLQESIKTGKLPTLSISTLDWFNYIMK

SEQ ID NO: 36 - protein, 1272 aa, synthetic construct
  source 1..1272: note = Description of Artificial Sequence: Synthetic polypeptide
MTPIFCNFVVYQIMLFNNNININVKTMNKKHLSDFTNLFPVSKTLRFRLEPQGKTMENIVKAQTIETDEERSHDYEKTKEYIDDYHRQFIDDTLDKFAFKVESTGNNDSLQDYLDAYLSANDNRTKQTEEIQTNLRKAIVSAFKMQPQFNLLFKKEMVKHLLPQFVDTDDKKRIVAKFNDFTTYFTGFFTNRENMYSDEAKSTSIAYRIVNQNLIKFVENMLTFKSHILPILPQEQLATLYDDFKEYLNVASIAEMFELDHFSIVLTQRQIEVYNSVIGGRKDENNKQIKPGLNQYINQHNQAVKDKSARLPLLKPLFNQILSEKAGVSFLPKQFKSASEVVKSLNEAYAELSPVLAAIQDVVTNITDYDCNGIFIKNDLGLTDIAQRFYGNYDAVKRGLRNQYELETPMHNGQKAEKYEEQVAKHLKSIESVSLAQINQVVTDGGDICDYFKAFGATDDGDIQRENLLASINNAHTAISPVLNKENANDNELRKNTMLIKDLLDAIKRLQWFAKPLLGAGDETNKDQVFYGKFEPLYNQLDETISPLYDKVRSYLTKKPYSLDKFKINFEKSNLLGGWDPGADRKYQYNAVILRKDNDFYLGIMRDEATSKRKCIQVLDCNDEGLDENFEKVEYKQIKPSQNMPRCAFAKKECEENADIMELKRKKNAKSYNTNKDDKNALIRHYQRYLDRTYPEFGFVYKDADEYDTVKAFTDSMDSQDYKLSFLQVSETGLNKLVDEGDLYLFKITNKDFSSYAKGRPNLHTIYWRMLFDPKNLANVVYKLEGKAEVFFRRKSLASTTTHKAKQAIKNKSRYNEAVKPQSTFDYDIIKDRRFTADKFEFHVPIKMNFKAAGWNSTRLTNEVREFIKSQGVRHIIGIDRGERHLLYLTMIDMDGNIVKQCSLNAPAQDNARASEVDYHQLLDSKEADRLAARRNWGTIENIKELKQGYLSQVVHLLATMMVDNDAILVLENLNAGFMRGRQKVEKSVYQKFEKMLIDKLNYIVDKGQSPDKPTGALHAVQLTGLYSDFNKSNMKRANVRQCGFVFYIPAWNTSKIDPVTGFVNLFDTHLSSMGEIKAFFSKFDSIRYNQDKGWFEFKFDYSRFTTRAEGCRTQWTVCTYGERIWTHRSKNQNNQFVNDTVNVTQQMLQLLQDCGIDPNGNLKEAIANIDSKKSLETLLHLFKLTVQMRNSVTGSEVDYMISPVADERGHFFDSRESDEHLPANADANGAFNIARKGLMVVRQIMATDDVSKIKFAVSNKDWLRFAQHIDD

SEQ ID NO: 37 - protein, 1313 aa, synthetic construct
  source 1..1313: note = Description of Artificial Sequence: Synthetic polypeptide
MNKGGYVIMEKMTEKNRWENQFRITKTIKEEIIPTGYTKVNLQRVNMLKREMERNEDLKKMKEICDEYYRNMIDVSLRLEQVRTLGWESLIHKYRMLNKDEKEIKALEKEQEDLRKKISKGFGEKKAWTGEQFIKKILPQYLMDHYTGEELEEKLRIVKKFKGCTMFLSTFFKNRENIFTDKPIHTAVGHRITSENAMLFAANINTYEKMESNVTLEIERLQREFWRRGINISEIFTDAYYVNVLTQKQIEAYNKICGDINQHMNEYCQKQKLKFSEFRMRELKKQILAVVGEHFEIPEKIESTKEVYRELNEYYESLKELHGQFEELKSVQLKYSQIYVQKKGYDRISRYIGGQWDLIQECMKKDCASGMKGTKKNHDAKIEEEVAKVKYQSIEHIQKLVCTYEEDRGHKVTDYVDEFIVSVCDLLGADHIITRDGERIELPLQYEPGTDLLKNDTINQRRLSDIKTILDWHMDMLEWLKTFLVNDLVIKDEEFYMAIEELNERMQCVISVYNRIRNYVTQKGYEPEKIRICFDKGTILTGWTTGDNYQYSGFLLMRNDKYYLGIINTNEKSVRKILDGNEECKDENDYIRVGYHLINDASKQLPRIFVMPKAGKKSEILMKDEQCDYIWDGYCHNKHNESKEFMRELIDYYKRSIMNYDKWEGYCFKFSSTESYDNMQDFYKEVREQSYNISFSYINENVLEQLDKDGKIYLFQVYNKDFAAGSTGTPNLHTMYLQNLFSSQNLELKRLRLGGNAELFYRPGTEKDVTHRKGSILVDRTYVREEKDGIEVRDTVPEKEYLEIYRYLNGKQKGDLSESAKQWLDKVHYREAPCDIIKDKRYAQEKYFLHFSVEINPNAEGQTALNDNVRRWLSEEEDIHVIGIDRGERNLIYVSLMDGKGRIKDQKSYNIVNSGNKEPVDYLAKLKVREKERDEARRNWKAIGKIKDIKTGYLSYVVHEIVEMAVREKAIIVMEDLNYGFKRGRFKVERQVYQKFEEMLINKLNYVVDKQLSVDEPGGLLRGYQLAFIPKDKKSSMRQNGIVFYVPAGYTSKIDPTTGFVNIFKFPQFGKGDDDGNGKDYDKIRAFFGKFDEIRYECDEKVTADNTREVKERYRFDFDYSKFETHLVHMKKTKWTVYAEGERIKRKKVGNYWTSEVISDIALRMSNTLNIAGIEYKDGHNLVNEICALRGKQAGIILNELLEIVRLTVQLRNSTTEGDVDERDEIISPVLNEKYGCFYHSTEYKQQNGDVLPKDADANGAYCIGLKGIYEIRQIKNKWKEDMTKGEGKALNEGMRISHDQWFEFIQNMNKGE

SEQ ID NO: 38 - protein, 1134 aa, synthetic construct
  source 1..1134: note = Description of Artificial Sequence: Synthetic polypeptide
MNELVKNRCKQTKTICQKLIPIGKTRETIEKYNLMEIDRKIAANKELMNKLFSLIAGKHINDTLSKCTDLDFEPLLTSLSSLNNAKENDRDNLREYYDSVFEEKKTLAEEISSRLTAVKFAGKDFFTKNIPDFLETYEGDDKNEMSELVSLVIENTVTAGYVKKLEKIDRSMEYRLVSGTVVKRVLTDNADIYEKNIEKAKDFDYGVLNIDEASQFTTLVAKDYANYLTADGIAIYNVGIGKINLALNEYCQKNKEYSYNKLALLPLQKMLYGEKLSLFEKLEDFTSDEELINSYNKFAKTVNESGLAEIIKKAVPSYDEIVIKPNKISNYSNSITGHWSLVNRIMKDYLENNGIKNADKYMEKGLTLSEIGDALENKNIKHSDFISNLINDLGHTYTEIKENKESLKKDESVNALIIKKELDMLLSILQNLKVFDIDNEMFDTGFGIEVSKAIEILGYGVPLYNKIRNYITKKPDPKKKFMTKFGSATIGTGITTSVEGSKKATFLKDGDAVFLLLYNTAGCKANNVSVSNLADLINSSLEIENSGKCYQKMIYQTPGDIKKQIPRVFVYKSEDDDLIKDFKAGLHKTDLSFLNGRLIPYLKEAFATHETYKNYTFSYRNSYESYDEFCEHMSEQAYILEWKWIDKKLIDDLVEDGSLLMFRVWNRFMKKKEGKISKHAKIVNELFSDENASNAAIKLLSVFDIFYRDKQIDNPIVHKAGTTLYNKRTKDGEVIVDYTTMVKNKEKRPNVYTTTKKYDIIKDRRYTEEQFEIHLHVNIGKEENKEKLETSKVINEKKNTLVVTRSNEHLLYVVIFDENDNILLKKSLNTVKGMNFKSKLEVVEIQKKENMQSWKTVGSNQALMEGYLSFAIKEIADLVKEYDAILVLEQNSVGKNILNERVYTRFKEMLITNLSLDVDYENKDFYSYTELGGKVASWRDCVTNGICIQVPSAYKYKDPTTSFSTISMYAKTTAEKSKKLKQIKSFKYNRERGLFELVIAKGVGLENNIVCDSFGSRSIIENDISKEVSCTLKIEKYLIDAGIEYNDEKEVLKDLDTAAKTDAVHKAVTLLLKCFNESPDGRYYISPCGEHFTLCDAPEVLSAINYYIRSRYIREQIVEGVKKMEYKKTILLAK

SEQ ID NO: 39 - protein, 1259 aa, synthetic construct
  source 1..1259: note = Description of Artificial Sequence: Synthetic polypeptide
MNYKTGLEDFIGKESLSKTLRNALIPTESTKIHMEEMGVIRDDELRAEKQQELKEIMDDYYRTFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYSVDFYDRELTQPGIEYYNGICGKINEHMNQFCQKNRINKNDFRMKKLHKQILCKKSSYYEIPFRFESDQEVYDALNEFIKTMKKKEIIRRCVHLGQECDDYDLGKIYISSNKYEQISNALYGSWDTIRKCIKEEYMDALPGKGEKKEEKAEAAAKKEEYRSIADIDKIISLYGSEMDRTISAKKCITEICDMAGQISIDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPIVHKKGSVLVNRSYTQTVGNKEIRVSIPEEYYTEIYNYLNHIGKGKLSSEAQRYLDEGKIKSFTATKDIVKNYRYCCDHYFLHLPITINFKAKSDVAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTILASTKWKVYTNGTRLKKIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 40 - protein, 1271 aa, synthetic construct
  source 1..1271: note = Description of Artificial Sequence: Synthetic polypeptide
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKRLEKIQEKKRKEIYQYLSDDAVFSEMFKEKMISGILPDFIRCNEGYSEEEKEEKLKTVALFHRFTSSFNDFFLNRKNVFTKEAIVTAIGYRVVHENAEIFLENMVAFQNIQKSAESQISIIERKNEHYFMEWKLSHIFTADYYMMLMTQKAIEHYNEMCGVVNQQMREYCQKEKKNWNLYRMKRLHKQILSNASTSFKIPEKYENDAEVYESVNSFLQNVMEKTVMERIAVLKNSTDNFDLSKIYITAPYYEKISNYLCGSWNTITDCLTHYYEQQIAGKGARKDQKVKAAVKADKWKSLSEIEQLLKEYARAEEVKRKPEEYIAEIENIVSLKEAHLLEYHPEVNLIENEKYATEIKDVLDNYMELFHWMKWFYIEEAVEKEVNFYGELDDLYEEIKDIVPLYNKVRNYVTQKPYSDTKIKLNFGTPTLANGWSKSKEYDYNAILLQKDGKYYMGIFNPIQKPEKEIIEGHSQPLEGNEYKKMVYYYLPSANKMLPKVLLSKKGMEIYQPSEYIINGYKERRHIKSEEKFDLQFCHDLIDYFKSGIERNSDWKVFGFDFSDTDTYQDISGFYREVEDQGYKIDWTYIKEADIDRLNEEGKLYLFQIYNKDFSEKSTGRENLHTMYLKNLFSEENVREQVLKLNGEAEIFFRKSSVKKPIIHKKGTMLVNRTYMEEVNGNSVRRNIPEKEYQEIYNYKNHRLKGELSTEAKKYLEKAVCHETKKDIVKDYRYSVDKFFIHLPITINYRASGKETLNSVAQRYIAHQNDMHVIGIDRGERNLIYVSVINMQGEIKEQKSFNIINEFNYKEKLKEREQSRGAARRNWKEIGQIKDLKEGYLSGVIHEIAKMMIKYHAIIAMEDLNYGFKRGRFKVERQVYQKFENMLIQKLNYLVFKDRPADEDGGVLRGYQLAYIPDSVKKMGRQCGMIFYVPAAFTSKIDPTTGFVDIFKHKVYTTEQAKREFILSFDEICYDVERQLFRFTFDYANFVTQNVTLARNNWTIYTNGTRAQKEFGNGRMRDKEDYNPKDKMVELLESEGIEFKSGKNLLPALKKVSNAKVFEELQKIVRFTVQLRNSKSEENDVDYDHVISPVLNEEGNFFDSSKYKNKEEKKESLLPVDADANGAYCIALKGLYIMQAIQKNWSEEKALSPDVLRLNNNDWFDYIQNKRYR

SEQ ID NO: 41 - protein, 1367 aa, synthetic construct
  source 1..1367: note = Description of Artificial Sequence: Synthetic polypeptide
MEKSLNDFIGLYSVSKTLRFELKPVSETLENIKKFHFLEEDKKKANDYKDVKKIIDNYHKYFIDDVLKNASFNWKKLEEAIREYNKNKSDDSALVAEQKKLGDAILKLFTSDKRYKALTAATPKELFESILPDWFGEQCNQDLNKAALKTFQKFTSYFTGFQENRKNVYSAEAIPTAVPYRIVNDNFPKFLQNVLIFKTIQEKCPQIIDEVEKELSSYLGKEKLAGIFTLESFNKYLGQGGKENQRGIDFYNQIIGGVVEKEGGINLRGVNQFLNLYWQQHPDFTKEDRRIKMVPLYKQILSDRSSLSFKIESIENDEELKNALLECADKLELKNDEKKSIFEEVCDLFSSVKNLDLSGIYINRKDINSVSRILTGDWSWLQSRMNVYAEEKFTTKAEKARWQKSLDDEGENKSKGFYSLTDLNEVLEYSSENVAETDIRITDYFEHRCRYYVDKETEMFVQGSELVALSLQEMCDDILKKRKAMNTVLENLSSENKLREKTDDVAVIKEYLDAVQELLHRIKPLKVNGVGDSTFYSVYDSIYSALSEVISVYNKTRNYITKKAASPEKYKLNFDNPTLADGWDLNKEQANTSVILRKDGMFYLGIMNPKNKPKFAEKYDCGNESCYEKMIYKQFDATKQIPKCSTQKKEVQKYFLSGATEPYILNDKKSFKSELIITKDIWFMNNHVWDGEKFVPKRDNETRPKKFQIGYFKQTGDFDGYKNALSNWISFCKNFLQSYLSATVYDYNFKNSEEYEGLDEFYNYLNATCYKLNFINIPETEINKMVSEGKLYLFQIYNKDFASGSTGMPNMHTLYWKNLFSDENLKNVCLKLNGEAELFYRPAGIKEPVIHKEGSYLVNRTTEDGESIPEKIYFEIYKNANGKLEKLSDEAQNYISNHEVVIKKAGHEIIKDRHYTEPKFLFHVPLTINFKASGNSYSINENVRKFLKNNPDVNIIGLDRGERHLIYLSLINQKGEIIKQFTFNEVERNKNGRTIKVNYHEKLDQREKERDAARKSWQAIGKIAELKEGYLSAVIHQLTKLMVEYNAVVVMEDLNFGFKRGRFHVEKQVYQKFEHILIDKSNYLVFKDRGLNEPGGVLNGYQIAGQFESFQKLGKQSGMLFYVPAGYTSKIDPKTGFVSMMNFKDLTNVHKKRDFFSKFDNIHYDEANGSFVFTFDYKKFDGKAKEEMKLTKWSVYSRDKRIVYFAKTKSYEDVLPTEKLQKIFESNGIDYKSGNNIQDSVMAIGADLKEGAKPSKEISDFWDGLLSNFKLILQMRNSNARTGEDYIISPVMADDGTFFDSREEFKKGEDAKLPLDADANGAYHIALKGLSLINKINLSKDEELKKFDMKISNADWFKFAQEKNYAK

SEQ ID NO: 42 - protein, 1229 aa, synthetic construct
  source 1..1229: note = Description of Artificial Sequence: Synthetic polypeptide
MEEKKMSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 43 - protein, 1261 aa, synthetic construct
  source 1..1261: note = Description of Artificial Sequence: Synthetic polypeptide
MIIHNCYIGGSFMKKIDSFTNCYSLSKTLRFKLIPIGATQSNFDLNKMLDEDKKRAENYSKAKSIIDKYHRFFIDKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQPTAISYRCINDNLPKFLDNVRTFKNSDVASILNDNLKILNEDFDGIYGTSAEDVFNVDYFPFVLSQKGIEAYNSILGGYTNSDGSKIKGLNEYINLYNQKNENIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSIESFSLADIQKYADVSESSETNAEVTEWLRNEIKEKCNLAVQGYESSKDLISKPYTESKKLFNNDNAVELIKNALDSVKELENVLRLLLGTGKEESKDENFYGEFLPCYERICEVDSLYDKVRNYMTQKLYKTDKIKLNFQNPQFLGGWDRNKEADYSAVLLRRNSLYYIAIMPSGYKRVFEKIPAPKADETVYEKVIYKLLPGPNKMLPKVFFSKKGIETFNPPKEILEKYELGTHKTGDGFNLDDCHALIDYFKSALDVHSDWSNFGFRFSDTSTYKNIADFYNEVKNQGYKITFCDVPQSYINELVDEGKLYLFQLYNKDFSEHSKGTPNLHTLYFKMLFDERNLENVVFKLNGEAEMFYREASISKDDMIVHPKNQPIKNKNEQNSRKQSTFEYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNLGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 44 - protein, 1242 aa, synthetic construct
  source 1..1242: note = Description of Artificial Sequence: Synthetic polypeptide
MKEQFVNQYPISKTLRFSLIPIGKTEENFNKNLLLKEDEKKAEEYQKVKGYIDRYHKFFIETALCNINFEGFEEYSLLYYKCSKDDNDLKTMEDIEIKLRKQISKTMTSHKLYKDLFGENMIKTILPNFLDSDEEKNSLEMFRGFYTYFSGFNTNRKNMYTEEAKSTSIAYRCINDNLPKFLDNSKSFEKIKCALNKEELKAKNEEFYEIFQIYATDIFNIDFFNFVLTQPGIDKYNGIIGGYTCSDGTKVQGLNEIINLYNQQIAKDDKSKRLPLLKMLYKQILSDRETVSFIPEKFSSDNEVLESINNYFSKNVSNAIKSLKELFQGFEAYNMNGIFISSGVAITDLSNAVFGDWNAISTAWEKAYFETNPPKKNKSQEKYEEELKANYKKIKSFSLDEIQRLGSIAKSPDSIGSVAEYYKITVTEKIDNITELYDGSKELLNCNYSESYDKKLIKNDTVIEKVKTLLDAVKSLEKLIKPLVGTGKEDKDELFYGTFLPLYTSLSAVDRLYDKVRNYATQKPYSKDKIKLNFNCSSFLSGWATDYSSNGGLIFEKDGLYYLGIVNKKFTTEEIDYLQQNADENPAQRIVYDFQKPDNKNTPRLFIRSKGTNYSPSVKEYNLPVEEIVELYDKRYFTTEYRNKNPELYKASLVKLIDYFKLGFTRHESYRHYDFKWKKSEEYNDISEFYKDVEISCYSLKQEKINYNTLLNFVAENRIYLFQIYNKDFSKYSKGTPNLHTRYFKALFDENNLSDVVFKLNGGSEMFFRKASIKDNEKVVHPANQPIDNKNPDNSKKQSTFDYELIKDKRFTKHQFSIHIPITMNFKARGRDFINNDIRKAIKSEYKPYVIGIDRGERNLIYISVINNNGEIVEQMSLNDIISDNGYKVDYQRLLDRKEKERDNARKSWGTIENIKELKEGYISQVIHKICELVIKYDAVIAMEDLNFGFKRGRFNVEKQVYQKFENMLISKLNYLCDKKSEANSEGGLLKAYQLTNKFDGVNKGKQNGIIFYVPAWLTSKIDPVTGFVDLLHPKYISVEETHSLFEKLDDIRYNFEKDMFEFDIDYSKLPKCNADFKQKWTVCTNADRIMTFRNSEKNNEWDNKRILLSDEFKRLFEEFGIDYCHNLKNKILSISNKDFCYRFIKLFALTMQMRNSITGSTNPEDDYLISPVRDENGVFYDSRNFIGSKAGLPIDADANGAYNIARKGLWAINAIKSTADDMLDKVDLSISNAKWLEYVQK

SEQ ID NO: 45 - protein, 1307 aa, synthetic construct
  source 1..1307: note = Description of Artificial Sequence: Synthetic polypeptide
MADLSQFTHKYQVPKTLRFELIPQGKTLENLSAYGMVADDKQRSENYKKLKPVIDRIYKYFIEESLKNTNLDWNPLYEAIREYRKEKTTATITNLKEQQDICRRAIASRFEGKVPDKGDKSVKDFNKKQSKLFKELFGKELFTDSVLEQLPGVSLSDEDKALLKSFDKFTTYFVGFYDNRKNVFSSDDISTGIPHRLVQENFPKFIDNCDDYKRLVLVAPELKEKLEKAAEATKIFEDVSLDEIFSIKFYNRLLQQNQIDQFNQLLGGIAGAPGTPKIQGLNETLNLSMQQDKTLEQKLKSVPHRFSPLYKQILSDRSSLSFIPESFSCDAEVLLAVQEYLDNLKTEHVIEDLKEVFNRLTTLDLKHIYVNSTKVTAFSQALFGDWNLCREQLRVYKMSNGNEKITKKALGELESWLKNSDIAFTELQEALADEALPAKVNLKVQEAISGLNEQMAKSLPKELKIPEEKEELKALLDAIQEVYHTLEWFIVSDDVETDTDFYVPLKETLQIIQPIIPLYNKVRNFATQKPYSVEKFKLNFANPTLADGWDENKEQQNCAVLFQKGNNYYLGILNPKNKPDFDNVDTEKQGNCYQKMVYKQFPDFSKMMPKCTTQLKEVKQHFEGKDSDYILNNKNFIKPLTITREVYDLNNVLYDGKKKFQIDYLRKTKDEDGYYTALHTWIDFAKKFVASYKSTSIYDTSTILPPEKYEKLNEFYGALDNLFYQIKFENIPEEIIDTYVEDGKLFLFQIYNKDFAAGATGAPNLHTIYWKAVFDPENVKDVVVKLNGQAELFYRPKSNMDVIRHKVGEKLVNRTLKDGSILTDELHKELYLYANGSLKKGLSEDAKIILDKNLAVIYDVHHEIVKDRRFTTDKFFFHVPLTLNYKCDKNPVKFNAEVQEYLKENPDTYVIGIDRGERNLIYAVVIDPKGRIVEQKSFNVINGFDYHGKLDQREKERVKARQAWTAVGKIKELKQGYLSLVVHEISKMMVRYQAVVVLENLNVGFKRVRSGIAEKAVYQQFEKMLINKLNYLMFKDAGGTEPGSVLNAYQLTDRFESFAKMGLQTGFLFYIPAAFTSKIDPATGFVDPFRWGAIKTLADKREFLSGFESLKFDSTTGNFILHFDVSKNKNFQKKLEGFVPDWDIIIEANKMKTGKGATYIAGKRIEFVRDNNSQGHYEDYLPCNALAETLRQCDIPYEEGKDILPLILEKNDSKLLHSVFKVVRLTLQMRNSNAETGEDYISSPVEDVSGSCFDSRMENEKLPKDADANGAYHIALKGMLALERLRKDEKMAISNNDWLNYIQEKRA

SEQ ID NO: 46 - protein, 1286 aa, synthetic construct
  source 1..1286: note = Description of Artificial Sequence: Synthetic polypeptide
MTNFDNFTKKYVNSKTIRLEAIPVGKTLKNIEKMGFIAADRQRDEDYQKAKSVIDHIYKAFMDDCLKDLFLDWDPLYEAVVACWRERSPEGRQALQIMQADYRKKIADRFRNHELYGSLFTKKIFDGSVAQRLPDLEQSAEEKSLLSNFNKFTSYFRDFFDKRKRLFSDDEKHSAIAYRLINENFLKFVANCEAFRRMTERVPELREKLQNTGSLQVYNGLALDEVFSADFYNQLIVQKQIDLYNQLIGGIAGEPGTPNIQGLNATINLALQGDSSLHEKLAGIPHRFNPLYKQILSDVSTLSFVPSAFQSDGEMLAAVRGFKVQLESGRVLQNVRRLFNGLETEADLSRVYVNNSKLAAFSSMFFGRWNLCSDALFAWKKGKQKKITNKKLTEIKKWLKNSDIAIAEIQEAFGEDFPRGKINEKIQAQADALHSQLALPIPENLKALCAKDGLKSMLDTVLGLYRMLQWFIVGDDNEKDSDFYFGLGKILGSLDPVLVLYNRVRNYITKKPYSLTKFRLNFDNSQLLNGWDENNLDTNCASIFIKDGKYYLGISNKNNRPQFDTVATSGKSGYQRMVYKQFANWGRDLPHSTTQMKKVKKHFSASDADYVLDGDKFIRPLIITKEIFDLNNVKFNGKKKLQVDYLRNTGDREGYTHALHTWINFAKDFCACYKSTSIYDISCLRPTDQYDNLMDFYADLGNLSHRIVWQTIPEEAIDNYVEQGQLFLFQLYNKDFAPGADGKPNLHTLYWKAVFNPENLEDVVVKLNGKAELFYRPRSNMDVVRHKVGEKLVNRKLKNGLTLPSRLHEEIYRYVNGTLNKDLSADARSVLPLAVVRDVQHEIIKDRRFTADKFFFHASLTFNFKSSDKPVGFNEDVREYLREHPDTYVVGVDRGERNLIYIVVIDPQGNIVEQRSFNMINGIDYWSLLDQKEKERVEAKQAWETVGKIKDLKCGYLSFLIHEITKIIIKYHAVVILENLSLGFKRVRTGIAEKAVYQQFERMLVTKLGYVVFKDRAGKAPGGVLNAYQLTDNTRTAENTGIQNGFLFYVPAAFTSRVDPATGFFDFYDWGKIKTATDKKNFIAGFNSVRYERSTGDFIVHVGAKNLAVRRVAEDVRTEWDIVIEANVRKMGIDGNSYISGKRIRYRSGEQGHGQYENHLPCQELIRALQQYGIQYETGKDILPAILQQDDAKLTDTVFDVFRLALQMRNTSAETGEDYFNSVVRDRSGRCFDTRRAEAAMPKEADANDAYHIALKGLFVLEKLRKGESIGIKNTEWLRYVQQRHS

SEQ ID NO: 47 - protein, 1264 aa, synthetic construct
  source 1..1264: note = Description of Artificial Sequence: Synthetic polypeptide
MENYGGFTGLYPLQKTLKFELRPQGRTMEHLVSSNFFEEDRDRAEKYKIVKKVIDNYHKDFINECLSKRSFDWTPLMKTSEKYYASKEKNGKKKQDLDQKIIPTIENLSEKDRKELELEQKRMRKEIVSVFKEDKRFKYLFSEKLFSILLKDEDYSKEKLTEKEILALKSFNKFSGYFIGLHKNRANFYSEGDESTAIAYRIVNENFPKFLSNLKKYREVCEKYPEIIQDAEQSLAGLNIKMDDIFPMENFNKVMTQDGIDLYNLAIGGKAQALGEKQKGLNEFLNEVNQSYKKGNDRIRMTPLFKQILSERTSYSYILDAFDDNSQLITSINGFFTEVEKDKEGNTFDRAVGLIASYMKYDLSRVYIRKADLNKVSMEIFGSWERLGGLLRIFKSELYGDVNAEKTSKKVDKWLNSGEFSLSDVINAIAGSKSAETFDEYILKMRVARGEIDNALEKIKCINGNFSEDENSKMIIKAILDSVQRLFHLFSSFQVRADFSQDGDFYAEYNEIYEKLFAIVPLYNRVRNYLTKNNLSMKKIKLNFKNPALANGWDLNKEYDNTAVIFLREGKYYLGIMNPSKKKNIKFEEGSGTGPFYKKMAYKLLPDPNKMLPKVFFAKKNINYYNPSDEIVKGYKAGKYKKGENFDIDFCHKLIDFFKESIQKNEDWRAFNYLFSATESYKDISDFYSEVEDQGYRMYFLNVPVANIDEYVEKGDLFLFQIYNKDFASGAKGNKDMHTIYWNAAFSDENLRNVVVKLNGEAELFYRDKSIIEPICHKKGEMLVNRTCFDKTPVPDKIHKELFDYHNGRAKTLSIEAKGYLDRVGVFQASYEIIKDRRYSENKMYFHVPLKLNFKADGKKNLNKMVIEKFLSDKDVHIIGIDRGERNLLYYSVIDRRGNIIDQDSLNIIDGFDYQKKLGQREIERREARQSWNSIGKIKDLKEGYLSKAVHKVSKMVLEYNAIVVLEDLNFGFKRGRFKVEKQVYQKFEKMLIDKLNYLVFKEVLDSRDAGGVLNAYQLTTQLESFNKLGKQSGILFYVPAAYTSKIDPTTGFVSLFNTSRIESDSEKKDFLSGFDSIVYSAKDGGIFAFKFDYRNRNFQREKTDHKNIWTVYTNGDRIKYKGRMKGYEITSPTKRIKDVLSSSGIRYDDGQELRDSIIQSGNKVLINEVYNSFIDTLQMRNSDGEQDYIISPVKNRNGEFFRTDPDRRELPVDADANGAYHIALRGELLMQKIAEDFDPKSDKFTMPKMEHKDWFEFMQTRGD

SEQ ID NO: 48 - protein, 1232 aa, synthetic construct
  source 1..1232: note = Description of Artificial Sequence: Synthetic polypeptide
MLHAFTNQYQLSKTLRFGATLKEDEKKCKSHEELKGFVDISYENMKSSATIAESLNENELVKKCERCYSEIVKFHNAWEKIYYRTDQIAVYKDFYRQLSRKARFDAGKQNSQLITLASLCGMYQGAKLSRYITNYWKDNITRQKSFLKDFSQQLHQYTRALEKSDKAHTKPNLINFNKTFMVLANLVNEIVIPLSNGAISFPNISKLEDGEESHLIEFALNDYSQLSELIGELKDAIATNGGYTPFAKVTLNHYTAEQKPHVFKNDIDAKIRELKLIGLVETLKGKSSEQIEEYFSNLDKFSTYNDRNQSVIVRTQCFKYKPIPFLVKHQLAKYISEPNGWDEDAVAKVLDAVGAIRSPAHDYANNQEGFDLNHYPIKVAFDYAWEQLANSLYTTVTFPQEMCEKYLNSIYGCEVSKEPVFKFYADLLYIRKNLAVLEHKNNLPSNQEEFICKINNTFENIVLPYKISQFETYKKDILAWINDGHDHKKYTDAKQQLGFIRGGLKGRIKAEEVSQKDKYGKIKSYYENPYTKLTNEFKQISSTYGKTFAELRDKFKEKNEITKITHFGIIIEDKNRDRYLLASELKHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYLLQSDTISKQSIASLVEGGCLLLPIINQDITSKERKDKNQFSKDWNHIFEGSKEFRLHPEFAVSYRTPIEGYPVQKRYGRLQFVCAFNAHIVPQNGEFINLKKQIENFNDEDVQKRNVTEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 49 - protein, 1067 aa, synthetic construct
  source 1..1067: note = Description of Artificial Sequence: Synthetic polypeptide
MKNGINLFKTKTTKTKGVDMEKYQITKTIRFKLLPDNAHEIVEKVKSLKTSNVDELMDEVKNVHLKGLELLFALKKYFYFDGNQCKSFKSTLEIKARWLRLYTPDQYYLKKSSKNSYQLKSLSYFKDVFNDWLFNWEESVSELAIIYEKYKICQHQRDSRADIALLIKKLSMKEYFPFISDLIDCVNDKNSNKTFLMKLSEELSVLLEKCNSRALPYQSNGIVVGKASLNYYTVSKSEKMLQNEYEDVCQSLDKNYDITEMKVILYKEKLDNLNFKDVTIANAYNLLKENKALQKRLFSEYVSQGKVLSLIKTELPLFSNINDNDFEKYKEWSNEIKKLADKKNTFCKKTQQDKIKDIQNKISELKKKRGALFQYKFTSFQKHCDNYKKVAVQYGKLKARKKAIEKDEIEANLLRYWSVILEQEDKHSLVLIPKNNAKDAKQYIETINTKGGKYIIHHLDSLTLRALNKLCFNAVDIEKGQMVRENTFYQGIKEEFERNKINCDNQGVLKIQGLYSFKTEGGQINEKEAVEFFKEVLKSNYAREVLNLPYDLESNIFQKEYTNLDQFRQDLEKCCYALHSKIGKDDLDEFTRRFEAQVFDITSIDLKSKKEKTKTTGEMKKHTQLWLEFWKGAIEQNFATRVNPELSIFWRAPKSSREKKYGKGSDLYDPNKNNRYLYEQYTLALTITENAGSHFKDIAFKDTSKIKEAIKEFNMSLSQSKYCFGIDRGNAELVSLCLIKNEKDFPFEKFPVYRLRDLTYQGDFKDKHDQMRYGVAIKNISYFIDQEDLFEKNNLSAIDMTTAKLIKNKIVLNGDVLTYLKLKEETAKHKLTQFFQGSSINKNSRVYFDEDENVFKITTNRNHNPEEIIYFYRGEYGAIKNKNDLEDILNEYLCKMETGESEIVLLNRVNHLRDAISANIVGILSYLIDLFPETIVALENLAKGTIDRHVSQSYENITRRFEWALYRKLLNKQLAPPELKENILLREGDDKIDQFGIIHFVEEKNTSKDCPNCRKTTQQTNDNKFKEKKFVCKSCGFDTSKDRKGMDSLNSPDTVAAYNVARKKFES

SEQ ID NO: 50 - protein, 1309 aa, synthetic construct
  source 1..1309: note = Description of Artificial Sequence: Synthetic polypeptide
MAKETKEFKTFDDFTNLYEVQKTLRFELEAVPETEIVLENRGIWYKRDKKRADEKPIVKFYMDILHREFTDEALEKIKESGVLNLSGYFKLFEELRRLQNHGANTKEEKKLKLEEIRAKKREISNELSQIRRVFSVRGFDVVDSDWKKKYTIEGKKIKNDKSKTYLILSENILNFLENRFTSKEVERLRSIDKKHVEDYGNVVNSGGENIFATFKGFFGYFDSLIKNRENFYETDGKAGRVATRSVDENLNFFAENLHIFSTDLPKALKDDLSDTQKAIFERSYYKNCLLQKDIKSYNLIIGDINKEINKHRQQRDTKIKFLNTLFKQILSIEEKEQYKHIEINNDEDLIRAIRDFISLNESKISEGTKIFNQFIQRCLQKEDLGQIYLPKDSVNTIAHRIFKPWDEIMALFDRKYFVSLEEIKDLTESSVWKERVLEESKTKSLIFKDTHIHTIISGQEIFSNFILILEKEYKNQFSGFISETRRGKAAFVGYDESLKNLRATIKWFEGKNLKLSETEKVEWIKAIKDYADAALRIFQMTKYLWLPVVGDEEDKDYLRIKAEIDQLTKDNDFYNKINAFIDGYKPEPFIYRSSFQEYLTRRPFSTDKFKINFENSRLLDGWDKDMIDDRMGILLQRDGDYFLGILNKEDRHCLDNLVDVKSEDKNSYALMQFKQLTGLYRQLPRMAFPKKKQPVLEANAEIKKIKEDFDFLQKQKKEREVNVNVVFDNKKLNLLINHYAEFLKENYKDEKCYDFSLLNKEKVYESLSDFYADVDKITYSLSFIQVSIDQLIKTGKILLFRLKNKDLLKGSLGQNKNLHTYYFHALFERENLSQGRIRLGAQAEIFFRPASIEKEKDKNRSNALKKSPKTRYVKEILKNKRYSEDKVFLHLPIQLNADAYDLPSINQNVFEFIKNRQEKVKIIGIDRGEKNLAYYSVISQNSNGKIKIEEPPRDLNLGYLEPLDELENKRQDERKAWQSISEIKSKRDGYISYAVSKIVELMLKYQAIIVLEDLSGKFKRSRMKFEKAPYQQLELALIKKLNYLVKKNSKSGKPGHYLSAYQLTEPVGSYKEMGKQTGIIFYTQAGYTSRTCPTCGWRKRVQGLYYKDRTSAQRRFDPKTGVKIFYDSVNDRFVFQYHPVYEQKELKEWDKEIYSDVTRIRWNNEEKKNNEYRKGDITLKIKRLFRDRGIDLSRNINEQLVNVGDASFWEELINLLRLITEIRNIDNENNRDFIECPHCHFQSENGFHGVAWNGDANGAYNIARKGLLITKAVCDPEKNVGDITWSDLKVDMKDWDAATDEWAKKNPEK

SEQ ID NO: 51 - protein, 1456 aa, synthetic construct
  source 1..1456: note = Description of Artificial Sequence: Synthetic polypeptide
MENEKIFSDLTNRYQVVKTLPFELKPVPRTRVLLGLDNPNKGEIFSKDRERAENFTIIKKYIDRLHSLFINESLKKADIDFSNFYKQYGKNINTKNNKNIDDDNDINDDEKEDSENDNLKKYRQEIANLFNKSKYKSWVNVGKDGDKISGMLFEKGLIDLLRTHFSDNLNEDIEIPELFSNKKIKDTRKLKEIINSFGKDGKDGQNFTTYFSVSFHNNRKNYYKSDGKMGRVSTRIVDENLERFCKNIYLYKEIIGKNEIKEIFSGNWDIYLQKKPNFSNDKTYKKLDEFKNDKYDWEMIFRDVNSYNKYFLQSDIEFYNYIRGKLNQDINEYNGKKRDSKEKINSQFENLRNQVHGEKKNYDDDFEIDEDNIIQFINEIFVRHNQNKMRFSEKLFSDFIDLLMVDNGDKLDKVYFSQKAVENAIARYYFVEETTNEGREPLLISLLLQNAGKDRKKLSNKPIKLGDIKFVLDQANNKPAEDIFKNRYVLSESNNDGIINANDKNHWANLLRLIKKDFYFHKDNLIKSQDKLALETKYNKGSDEGERQIETIKNFAESAKAILRMTKYFDLRKNGVIQNVIGGKDPIHEEVDKYFDGDVLSGEESCRISKYYDALRNFITKKAWSADKIILNFDCSEFLGGWDRSQEQKKRGIILRHRDGDEERYYLAVLGKNGKQYFENRTLFKGCESSDWQKIEYNVIQKPHMSLPKNLITPFFKKDKITNERFIDRSKKGAKALIEIDINPSDEFLNNYNLGKHTKENLDKSFLCDYFKYLMDAIAKYYKGEFNFNFPDVSNFDNTQPFYSFIEKNAYSIKYFGISSKEIEKLIADCYYKEDVYLFQIYCKDFEIDPKIGKAKYGNEFRTKAEIRKSKGEEAGNENLNTKYFKLLFDEKNLKNQNGIVYKLNGGAKMFYRPSSIKKDEKIDGKWRYKEDKYSLNITITCNFSSKKDDLSIDKDINKKIAEVNANSDFRIISIDRGEKNLAYCCVMDENANILDIKSLNRITRYDKNGKAIKEKNMFHEVKDGKLCYGEPVYDFYKDYQNLLDEREIKRLVNRRSWNVIEDIKNLKKGYVALLINYICKAVVIAINEGKYPIIVLESLDKGMLHNRVKIEKQIYRGVEEGLVRKLNYFVDKKTDNVLNAWQLLAKFETVGSSLDRKKQLGIIFYVDPGYTSITCPCCGFRQRKYIKAERAEENFKEIKIKFDGKRYSFAYDYRCIDDNGKEKSKEDIIYSNVKRLLRSGRNGRAVQIEDVTDELTNLFKKHNINIEQDINEQLAGKDNKFWKQLLWWFNAIEQIRNTQSLRRKFNTEENKLEILENNDCDFILCPHCYFDSNKDKFQNKIWNGDANGAFNIGRKGIIDIFEIKKHQRMLSDFMEQWGIDKLPKANGGNQAVIEIVKNDKKYNLCILNNKKIPYYCLRIGKEKIDSIADDRKCNQLPDLMVNWKKWDMWLDKWGK

SEQ ID NO: 52 - protein, 1486 aa, synthetic construct
  source 1..1486: note = Description of Artificial Sequence: Synthetic polypeptide
MPEVKNVFQDFTNLYELSKTLRFELKPVPETEKILELNAAKTKKFPKDLYRAENFEIIKKYTDELHRTYIRETLNNVNIDYLKFLEIFRINGKKKNEMTDENEESDENNEKDDIQKIKKELRSKIGNLFNKWNNDKDNKFKDWVKIDVGKKEKEVSGDLFGKELITILKNYFKNKLDSKVNVPMLFFNEQEIKNGEAKKQRKLEAVFENFDKFTTYFTDSFYNNRKNYYKTEGRVGQVATRIIDENLPRFCSNLIAFNEVVSLYSTLLNNFDLGWKEYLNEKKINQTWVEKFELSNYDWKALFNDVNYYNQCLLQEGIDKYNYIIKKLNKDINEYTQNKYKSVEKGNNNNPDINFFQKLHKQIHGERDFKLIEIDIDENNIFTKILPEFILHSDMKLMTKIDEEVGVEEIVGAERIIKIFIKQELKDLEKIYLSRRAIETISAKWFHSWETLKDLILGYLNKDLLESKKRKKVPDFVDFNIIKIVLENNKDDYKDLFKRKYFEADKNEFVDWIDSSGGTKKLEFGGENWINFLNVFEYEFGTLLTEYKKNKNALLYLIDKKIDYDKNNEVGQTAAIKNFADSALGIFRMVSYFALRKKGVMVEPKNGKDEIFYAFVDRYLDGDDNDREEQNKIVQYYNTLRNFVTQKAWSIDKVRLCFDCGEFLKGWDKDKIHERLGIILRNNNKFYLGILNKNHKQIFIKIKSHDNNNFYYVIYDYKQLNNVYRQIPRLAFPSRSVKKGDAYMLRAIQERKKKFFLEDEEFIELQEIKNEYDKIGNDLSKEKLTKLIEYYKKVVISNYSSLYNVSNLNNKKFNSINEFNQYVENLMYSLIPTRISPDFIKEKISKGELYLFQIYNKDFELDESIGKEKFGEDFAPVIMDGKNNLHTEYFKLLFNDSNLKNPNGVVFKLSGGAKMFYRPATENLPIKKDRDGNIIKNKKGENVIVGQRYKEDKYFLHLPIILNFVNKGKNYSINDMVNKAITNASDDQDKFRIIGLDRGEKHLVYYSVINERQEIIEIGSLNNISRKDNKGEIIEEKNWYHDKFGNIEKEPTKEYHKDYHNLLDQREIERLKSRQSWEKIENIKELKEGYISAVINKICNLVIKAIKENKIPIVALENLNSGMKRGRIKIDKQIYQKLELKLAKKLNFLVDKKEKNYLSAWQFTPKIETFSGDIEKKNQVGIIFYVDPAFTSATCPNCGFRKRIKMDPQNAKKKIKDMEITYENGIYKFDYPIENGENDVVYSDVERLKWDNEKKKVIKTKNVSDDFGKLFEDIKDKNNLKKELLSIGEENKEFWKEFSRCFNLLLRIRNSKLIKRKLNDDTGKVEIIADDDLADRDRDFIYCPQCHFHSEGGDVFGEFVKKKYLGKDNFEFNGDANGAYNIARKTIIAVNKIKDYQLGLNHFIEKYRISELPNNGKDKKNIFYNNNSYILSFFEVQDEKFRKVKVYGLKKDGDRQIIQKKEMWYRRYPDIFVNNKEWDKFVQNKS

SEQ ID NO: 53 - protein, 1403 aa, synthetic construct
  source 1..1403: note = Description of Artificial Sequence: Synthetic polypeptide
MLFFMSTDITNKPREKGVFDNFTNLYEFSKTLTFGLIPLKWDDNKKMIVEDEDFSVLRKYGVIEEDKRIAESIKIAKFYLNILHRELIGKVLGSLKFEKKNLENYDRLLGEIEKNNKNENISEDKKKEIRKNFKKELSIAQDILLKKVGEVFESNGSGILSSKNCLDELTKRFTRQEVDKLRRENKDIGVEYPDVAYREKDGKEETKSFFAMDVGYLDDFHKNRKQLYSVKGKKNSLGRRILDNFEIFCKNKKLYEKYKNLDIDFSEIERNFNLTLEKVFDFDNYNERLTQEGLDEYAKILGGESNKQERTANIHGLNQIINLYIQKKQSEQKAEQKETGKKKIKFNKKDYPTFTCLQKQILSQVFRKEIIIESDRDLIRELKFFVEESKEKVDKARGIIEFLLNHEENDIDLAMVYLPKSKINSFVYKVFKEPQDFLSVFQDGASNLDFVSFDKIKTHLENNKLTYKIFFKTLIKENHDFESFLILLQQEIDLLIDGGETVTLGGKKESITSLDEKKNRLKEKLGWFEGKVRENEKMKDEEEGEFCSTVLAYSQAVLNITKRAEIFWLNEKQDAKVGEDNKDMIFYKKFDEFADDGFAPFFYFDKFGNYLKRRSRNTTKEIKLHFGNDDLLEGWDMNKEPEYWSFILRDRNQYYLGIGKKDGEIFHKKLGNSVEAVKEAYELENEADFYEKIDYKQLNIDRFEGIAFPKKTKTEEAFRQVCKKRADEFLGGDTYEFKILLAIKKEYDDFKARRQKEKDWDSKFSKEKMSKLIEYYITCLGKRDDWKRFNLNFRQPKEYEDRSDFVRHIQRQAYWIDPRKVSKDYVDKKVAEGEMFLFKVHNKDFYDFERKSEDKKNHTANLFTQYLLELFSCENIKNIKSKDLIESIFELDGKAEIRFRPKTDDVKLKIYQKKGKDVTYADKRDGNKEKEVIQHRRFAKDALTLHLKIRLNFGKHVNLFDFNKLVNTELFAKVPVKILGMDRGENNLIYYCFLDEHGEIENGKCGSLNRVGEQIITLEDDKKVKEPVDYFQLLVDREGQRDWEQKNWQKMTRIKDLKKAYLGNVVSWISKEMLSGIKEGVVTIGVLEDLNSNFKRTRFFRERQVYQGFEKALVNKLGYLVDKKYDNYRNVYQFAPIVDSVEEMEKNKQIGTLVYVPASYTSKICPHPKCGWRERLYMKNSASKEKIVGLLKSDGIKISYDQKNDRFYFEYQWEQEHKSDGKKKKYSGVDKVFSNVSRMRWDVEQKKSIDFVDGTDGSITNKLKSLLKGKGIELDNINQQIVNQQKELGVEFFQSIIFYFNLIMQIRNYDKEKSGSEADYIQCPSCLFDSRKPEMNGKLSAITNGDANGAYNIARKGFMQLCRIRENPQEPMKLITNREWDEAVREWDIYSAAQKIPVLSEEN

SEQ ID NO: 54 - protein, 1356 aa, synthetic construct
  source 1..1356: note = Description of Artificial Sequence: Synthetic polypeptide
MTIKKHKPFTNFECLTPVQKTLRFRLIPVGRTTEFVKCRNIIEADRKRSEMYPLLKELADRFYREFMTDQLSNLLFDWSPLVEALLLARNNTDPRENQRIASLVRDEQKKYRTLLLKRLSGQVDRNGTPLPKNTASVNKKYYDDLFKARFVTETLPAYLEHLKNKPDGRISDELFDAYKDALDSYQKFTSRLTNFWQARKNIFTDEDIATGFAYRIVHEIVPDYLFNRRVYEQHKLDFPEPLDLLETELKKKNLIANDESLDALFTIPAINRLLTQKGVDLHNAVIGGFFTDDHTKVQGFNELANLKNQTLKNVSDNSEIKPVGKMTRLKKHILSISESTSFLFEQIESDDDLLARIIEFNNTLSEPDIDGLSIADINDQLYNIMTGVDPSTILVHARNLNKLSHEASLSWNRLRDGLYQMATESPYREDERFKRYIDASEEERDLSKLKNDIYFSLQELQFALDQSIDLEEEATPTEDIFLPFEFPGMDLKSELTVLFRSIEQLISSETKLIGNPDAIATIKKYLDAIMARYSIWNLLSCEAVELQDDLFYPEYDRVMGSLSNIILLYNLARNYLSRKPSSKEKFRLNFDKPTLADGWSESKVPDNFSVLLRKDDLFYLGILKDRKAYRVLSYENCDETAKNIKGYYERMIYHFSPDAYRMIPKCSTARKDVKKHFGEQGETTGYTLYPGASNFVKPFTIPYEIYRLQTELVNDKKRYQADYLKQTEDEEGYRQAVTAWIDFCKSYLESYEGTSTFDYSHLLKSEDYEDVNQFYADVDRASYSIYFEKVSVDLIHTMVDRGDLYLFQLYNKDFSPHSTGKPNLHTMYWRALFSNDNLQNNTIKLNGQAELFYRPKQVEQPTVHLQGSYLLNRFDKHGDVIPAGLYCEIYNHINERHPEGYTLSEEATQGLLDGRFVYREAPFELVKDKRYTEDQLFLHVPLEFNWTASANVPFENLANEYIKKDSDLHIIGIDRGERNLLYYSVINLQGDIVKQGSLNTLIQQTTLKGETVERQIPYQSMLKQREDERAEARQNWQSIDRIKDLKEGYLSHVIYKLSRLIIKYHAIVVMENLNVGFKRGRFKVERQVYQKFEVALINKLNALSFKEYEPNELGGVMRPWQLARRVVSPEDTRSQNGIVFYVPASYTSIVDPVTGFANLFYLNRIRNKDLNSFYGHFQEIRYDHEFDRFIFRFNYADFGVFCRIKNVPSRTWNLVSGERKAFNPKRRMIEKRDTTDEIKKALEAHGIAYQNEQNLLPLLLENENLLARIHRSFRLVLQLRNSDSDRDDIVSPALDKENNTFDSGQQPYESSLPINADANGAYNIARKGLLLVDKVKNDKRAVLSNREWFEYLMAEE

SEQ ID NO: 55 - protein, 1296 aa, synthetic construct
  source 1..1296: note = Description of Artificial Sequence: Synthetic polypeptide
MENKDYSLSRFTKQYQNSKTVRFALTPIGRTEEYIIQNQYIEAARRKNQAYKIVKPIIDEKFRSMIDDVLTHCEKQDWVTLDKLILQYQNNKCRENMDALAEQQEEIRKNISEEFTKSDEYKNFFGKEDSKKLFKIFLPEYLNQINASESDKEAVNEFQKFKTYFSNFLIVRADIFKADNKHNTIPYRIVNENFMIFAGNKRTFSNIIRLIPNALEEIAKDGMKKEEWSFYNIQNVDSWFEPDSFQMCMSQKGIQKYNFIIGLVNSYINLYTQQNPQATEVKRSRLKLRMLHKQILSDRVNPSWLPEQFKEGEEGEKQIYEAILALENDLIKNCFDKKYDLWIQSIDIQNPRIYIAASEMARVSSALHMGWNGLNDVRKTILLKSDKKQAKVEKILKQDVSLKDLSDTLNRYADIYKEEQIPSLYQYIEYGSELLQDCAITRKEYHDLLNGNSNTLSLNQNEKLIEGLKAYLDSYQAIVHFLNVFIVGDELDKDTDFYAELDGLVESLSEIVPLYNKVRNYITRKVYSLDKMRIMFERSDFLGGWGQSFDTKEALLFQKDNLYYIGIIEKKYTNMDVEYLHEGIKEGNRAIRFIYNFQKADNKNIPRTFIRSKGTNYAPAVRKYNLPIESIIDIYDVGKFKTNYKKINEKEYYESLEKLIDYFKDGILKNENYKKFHFNWKPSNEYENINEFYNDTNNACFLLEKEEINYDHLKEQANQGKIYLFQISSKDFNEGSKGTPNLQTMYWRELFSNQNCKDGVIKLCGGASIYMRDASIKQPVVHRKNAWLINKWYKVNGQNVVIPDNTYVKFTKIAQERMNEDELTPQERQLWNSGLIQKKKATHDIMKDRRFTKKQYMLHAPLTINYKQQDSPRYFNEKVRSFLKDNPDINIIGIDRGEKNLIYITIIDQKGNILKGMQKSFNQIEEKGKEGRTIDYYSKLESVEARHDAARKNWKQIGTIRELKEGYLSQVVHEITQLMIQYNAVIVMENLNMGFKKGRMKVEKSVYQKFEKMLIDKMNYLAFKRDMQGNAIDPYEVGGVMNGYQLTDRFTSFADMGSQNGFIFYVPAAYTSVIDPVTGFVNVFQKTEFKTNDFLHRFDSISWNDKEQSFVFTFDYQNFKCNGTCYQNKWSLYADVDRIETIIKNNQVDRIEPCNPNQKLIDFFDKKGIIYRDGHNIVDDLEKYDSKTISEIIHNFKLILQLRNSMRNPDTGEIIDYIASPVMHNEERFDSRKRNPELPQDADANGAYHIALKGLMFLQKINEYADSDGNMDNRKLKITNEEWFKYMQTRKEHTYF

SEQ ID NO: 56 - protein, 1250 aa, synthetic construct
  source 1..1250: note = Description of Artificial Sequence: Synthetic polypeptide
MSNKTSSITTTNKLSYTGFHNNGKQSKTLMFELKPIGRTTEHLDRKGYLADDIDRAESYKTFKEIADNFHKNLIEESLATFTFSDTLKDYFDLWLSPVRTNEDTPKLRKMEAKLRKELSSALKQHPSFAATSSGKRLIDEALYPNASDKERQCLDRFKGRSSYLDSYTEVRSFIYTDLCKHNTIAYRVVNENLKIYLENILAYEKLMQTAVNGKLETVKEMFHDLYPTFSMDISIFFTSYGFDYCLSQNAITRYNILLGGWSDDNGIHHKGLNNYINEYNQTVPRNKRLPKLNKLQKMILSEENSMSFIIDKFENDVDLANAIRYWLKNCQFDALNLLIWTLDVHYNLDEIHFKNDNQGKNISDLSQALFKNHHVIRDAWDYDYDIVNAKAKSRQKPERYAEKRDKAFKKINSFSLSYLANILSQYDNQYANFVAQFKTRISVHIQNVQQMIADKTLDMRLDPLMLLKSISSDTKLVEDIKRVLDSLKDMQRMLTPLLGEGTEPNRDAMFYSDFEPLMNYVDTLTPLYNKVRNYITKKPYSTKKTSLYFGASNFGSGFDVTKLPVSHTIIMRDKGCYYLAVIDNNKLIDKLYDHNDNDGYEYMVYKQIPSPIKYFSLKNILPQDPPDDIRQLLEDRKNGAKWSHDDETRFIDYIVNEFLPTYPPIHDKNGNPYFSWKFKNPDEYESLNEFFDDVSKQAYQTSFRFVSRDFVDDAVENGDIFFFQIYNQDFSPASHGKPSPHTLWFRALFSDVNLETKDIRLKGNATAYFRPASIFYTDEKWRKGHHYEQLKNKFKFPIIKDKRYALDKFFFHITLEINCNATVEKYFNNRVNEEIRKADRYNILAINRGERNLLYAVVMDQDGTILEQKSFNIIKSELPNKTVKETDYWKKLHAREKERDTARKSWKSIECIKDLKKGYLSYVVKTITDMMFEYNAVLVMENLDIEMKRSRQKIEKNVYAQFQNAIIQKLSMYVNKDIDLHIARTAPGGTLNPYQLTYIPASRTKTPKQNGFVFFLNPWNITEIDPTTGFVDLFQTCFRTKNEYKDFFAKFKDIRYNEAQGWFEFDTDYTYFRDKEKAGKRTRWNICSYGTRLRRFRNPDKNYAEDAMTVYPTQMLKDLFDEYNIPYAPASAKSTSISIKDDIIQIDKLDFYKKLLYILKLIVQLRNTSPSSTEQEDDYIISPVINEDTNWFYDSRDYNEESLLPCNTDANGAYSLALKCNMVIDRIKNTIPGEPVDMYISNADWLDARQ

SEQ ID NO: 57 - protein, 1328 aa, synthetic construct
  source 1..1328: note = Description of Artificial Sequence: Synthetic polypeptide
MNSKTSIFDFSNIFGRDITLRFKLTPVTINSKGEVKDANGADPYRPYLSADEELQEQYELLKTAIDAYHQMYIDKKLKHILCLPLTEKGKDGVEHDTAKSKFVKSCLAYIKDYGEKDKKRQTADLRTFISRVFADDNISSLPPYKVKSDFITKTLRQWLEQPDTKVEKKEAILDLIEKNGSKLYANCQGLLEARQRLYEKDGKSTSVPYRCIDRNLPRFSKDYHLFEKILGDCSDVFDFEQLDKDFSEELKGIARLSGIRVESVREVFQPLLYLAYLNQEGIQYLNTIIGTKKEKGTSALGLNEYINQYNQKQGIKKKKDGIPMLNKLNNQILFGDEVFIETLAEHKEAIPVIKKVVSSLGKLGAFDGECHENKLYQFLLSLSSYAGNIYVNTKVVAQISSSLWGDYSILYDAVKHDKNGRLIQKSVTLGELNEKIERLKLEDNRDAFEYFRRSQVKDVVHGSSNVGVFEQLKNCYNDFVEKKILKCSFFSEDQVLVIQRLFDSILSLQRIFKVFCPSLYEVDSDGLFVAKFSDYWNVLRGFDKDYDLLRNLFKRKPYSTDKIRVHFGLSNLMDGFVDSWTDKKDKGTQYNGYILRQAHSFVDENTSKELQEFQRYNYYLVISGNVRLFREKGNALVCEKKKEKLVASDEFSGFERFDYYQSSINNFNREFKRLTGRDRKSFTDEILQNEGKKELKSTYIENLIKVAKSMKRLTALQNLVSDEKVRKYSENLDYETLSAEIGQILATGRERKYVPVSTNEMKNLLKSSKNNKGEEVRTFMFRISNKDLSYAETMQKGERKSHGAENMHTMYFRALLDTLQNTFDIGTGTVYFRKASDKRKMKYDEKNPTHRKGDELAFKNPYNKGKKKSVFGYDLIKDRRYTKDSYLFHLSITQNYQKKGNAEDLNAMVRDYIRTQEDLRVIGIDRGERNLLYATMIDGEGHILAQKSFNVIGYQGTTASGESFQVETDYHQLLNEKAEKMRSLQREWKEMDKIQDMKDGYLSVVVHELAKMVVENNAIIVMEDLNMGFMESRQSQLANVYQKFEEKLRNKLQFYVDKRKRNDEPSGLYHALQLAGTETKDNQNGFIFYIPAWNTSKIDSVTGFVNLFNLKYTNIKDAKAFFSTFEKIEKNVETGHYDFTFSYSSMARKKMAKRMDGTRDSWTISTHGSRIVREQKGNYWEYREIESLTSEFDALFEKYSIDTRCRLKEAIDKCGEAEFFKELIRLMKWTLQLRNYDDRGNDYIVSPVCYRGNEYYCSLDYDNEEGMCISKIPCQMPKDADANGAFNIARKGLMLCERLKKGEKIGVIKGTEWLQYVQNMSERYVGMV

SEQ ID NO: 58 - protein, 1434 aa, synthetic construct
  source 1..1434: note = Description of Artificial Sequence: Synthetic polypeptide
MINTMEQPKKSIWDEFTNLYSLQKTLRFELKPQGKTKELVRTLFINPEEHHHKLISDDLELSKNYKKVKKLIDCMHRNIINNVLSKHQFTGEELKKLDKNSNAEDNDTETDNADKKDPFAKIRERLTKALNEESKIMFDNKLLNPKKGKNKGECELKKWMDKAEDKYFELGNNEKIDKEAVKADMERLEGFFTYFGGFNKNRENVYSSKKIATAIPFRIIHDNFPIFKKNIENYKKITEKHPELAKLLNEKGANEIFQLEHFNKCLTQDGIDVYNNEKLGIIAKEQGKEQDKGINQLINEYAQKKNKEIKENAKGGEKPKKIKIAVFDKLKKQILSISKTKSFQFEVFEDTSDIINGINKRYTFLTEAKEGMSIVDEIKKIIGSVGDEKYSLDEIYLKEKFISTLSKKLFNYSRYIEVALEKWYDDRYDDKINKSGTDKRKFISAKQFSITSIQDAINYYLEKYEKDEELSKKYTGKNIIVDYFKNPTITIEHKQKEEVISEEKDLFKELEVRRNVIQHILNGDYKKDLKEEKQQDGDSEKVKAFLDALLEFNYILNPFIIKDKNLRKEQEKDEEFYNEIKKLQESIFEAEILDLYNQTRNYITKKPYKLDKFKLTFGSGYFLSGWSNDMEEREGSILIKYNEDRSKNYYLIIMAKPLTDDDKKQLFSDNGTHSKICIYEFQKMDMKNFPRMFINSKGSNPAPAIEKYNLPIKTIWADYQKYKNLNQKGKDKFLEENPDFRHNLIGYFKICAEKHESLAPFKHQFSSIWKPTKEYENLAQFYKDTLEACYNLKFENVNFDNISQLVSSGKLHLFKIHNKDFNPGSTGKKNLHTLYWEMLFDEKNLQDVIFKLSGGAELFYREASILKNKIIHKIGEKVLKKFFKLPDGKLEPVPAESIKNLSAYFRKELPEHELTEIDRKYIDNYSIIGKKDDKLGIMKDERFTVDKIQFHCPITINFKSKNKNFINDDVLEYLHKRDDVHIIGLDRGERHLIYLTMINKDGKIVDNMQFSLNELQRRYKINGNEEIQKINYQKLLDTREVSRTEARRNWQTIENIKNLKEGYLSLIVHQLAKLMIEKNAIVVMENLNYGFKDSRARVEKQIYQKFESILIKKLQYLVMDKNNLYDSGGVLSAYQLTNQEVPAYKYISKQNGFLFYVPPDYTSKIDPETGFINLLDTRYYSRKNAVALLNKFDKIYYDRDNKYFRFDFDYNSTDSNGNKNFDKLRVDISELTRTKWSVCSHPAKRSITVQINNKWVRQPINDVTDKLIKLFEDKQIGYESGKCLKDEILKVEDAKFFEDLLRYLSVLLALRHTYTENGVEYDLIISSVEKAPGSNEFFVSGKDNNLPANADANGAYNIARKGLWLLRKLDEIDNQELAIKKFNELKHAKEIKKNGEESKEDKGDRKRKKKWVSQWCPNKEWLAFAQSMQDVSEK

SEQ ID NO: 59 - protein, 1263 aa, synthetic construct
  source 1..1263: note = Description of Artificial Sequence: Synthetic polypeptide
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 60 - protein, 1283 aa, synthetic construct
  source 1..1283: note = Description of Artificial Sequence: Synthetic polypeptide
MSNLNTFISPEFTGKIKMTKSLKVSMIPIGETEHWIAKHKVFEKDRELFDKNLKARPILDEFIKYTVSRALPNLLFDFEAYYLVKKDRTKARAFEKELAKTVTDLILKEMDELKSASLIDSADFVKTTLKKFAGTHDIPGLSRIEAIESLEAASKLTALNGKFNTSRIAIINTLIPKRIIENFDIYLSNMEKIRNVYESGEFGFLFERYPDTLLFMEPANYRTVCSPEAIEDYNRFISGYGDSTESWIKGFNQELSEASNSSKSSNGGVRRYSLIKPLHKQHLFETKKFFTFASISSDDDVRELINSVKGSTEDACLNALAFFSSSDPKTLFVKGSYLHTLSAFLYGSANSYILPERIKEGEKARLTAEYDSVAKKTKAVTTRYNVAMNNISKKINEKIFSLADIDAYCCDISKRRSVREILLGIMQEMYAAVYGENGKWSNIEAEAVLDSKTKIWKAKNGAVAKAVNDYLTAILEIRKFIRPFALRMEELEELGLDTSSALDAGEITNTLFEAVRAQKLVHAYLTRNDADIALSTQVYFGGTQKAAASWWNYETGDIQNRQIALAKKDGMYYFIGTFDERGSYSIEPASPGEDYYEMLDVKKGQDANKQIKKVLFSNKAIREHFADSSNDYVITTKVNSPITVRREIFDKYQAGEFKLTSQKIRKGDLVGEKEMTYYREYMDLLFQMAKGYTEYSRFNMDTLLPIEEYDTENDLLDDVNTNTIDYRWVRISAACIDDGVRNGDIFVFRAQTSSMYGKRENKKGYTGLFLELVSDENLLVTRGMSLNSAMSIYYRAKVHDAITVHKKGDVLVNKFTNARERIPENSYKAICAFYNSGKSIEELTIEDRDWLAKATTRICSGEIIKDRRYTKNQYSISISYNINRSVNNRKRVDLATIVDDTASAGRIISVTRGTKDLVYYTVIDDGGSVIEARSLNVINGINYAKMLAQISEERHDSNANFDIPKRVETIKEAYCAFAVHEIISAALKHNALIVVELISDAIKDKYSLLDNQVFLKFENVLKNCLMSVKVKGARGMEPGSISNPLQLCNADDKSFRNGILYQIPSSYINICPVTGYADIIDYYNIVSAGDIRNFFVRFENIVYNKEKARFEFSFDLKNIPIKLEKCPDRTKWTVLGRGEITTYDPLTKSNHYVFDAAQMLAETVSKEGLDPCANIVEHIDELSAATLKKMFNTFRNIAKGIVSECDEVPVSYYKSPVIDEADIKNKSLDNKSISEIKCYNLDEKARYYLALAKSSSDGENKNRYVSSTAIEWLNYIQEKRTHE

SEQ ID NO: 61 - protein, 500 aa, synthetic construct
  source 1..500: note = Description of Artificial Sequence: Synthetic polypeptide
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 62 - protein, 507 aa, synthetic construct
  source 1..507: note = Description of Artificial Sequence: Synthetic polypeptide
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 63 - protein, 529 aa, synthetic construct
  source 1..529: note = Description of Artificial Sequence: Synthetic polypeptide
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 64 - protein, 726 aa, synthetic construct
  source 1..726: note = Description of Artificial Sequence: Synthetic polypeptide
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 65 - protein, 517 aa, synthetic construct
  source 1..517: note = Description of Artificial Sequence: Synthetic polypeptide
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 66 - protein, 481 aa, synthetic construct
  source 1..481: note = Description of Artificial Sequence: Synthetic polypeptide
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 67 - protein, 358 aa, synthetic construct
  source 1..358: note = Description of Artificial Sequence: Synthetic polypeptide
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 68 - protein, 507 aa, synthetic construct
  source 1..507: note = Description of Artificial Sequence: Synthetic polypeptide
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 69 - protein, 534 aa, synthetic construct
  source 1..534: note = Description of Artificial Sequence: Synthetic polypeptide
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 70 - protein, 537 aa, synthetic construct
  source 1..537: note = Description of Artificial Sequence: Synthetic polypeptide
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 71 - protein, 540 aa, synthetic construct
  source 1..540: note = Description of Artificial Sequence: Synthetic polypeptide
LWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 72 - protein, 542 aa, synthetic construct
  source 1..542: note = Description of Artificial Sequence: Synthetic polypeptide
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 73 - protein, 564 aa, synthetic construct
  source 1..564: note = Description of Artificial Sequence: Synthetic polypeptide
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 74 - protein, 610 aa, synthetic construct
  source 1..610: note = Description of Artificial Sequence: Synthetic polypeptide
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 75 - protein, 369 aa, synthetic construct
  source 1..369: note = Description of Artificial Sequence: Synthetic polypeptide
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 76 - protein, 552 aa, synthetic construct
  source 1..552: note = Description of Artificial Sequence: Synthetic polypeptide
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 77 - protein, 534 aa, synthetic construct
  source 1..534: note = Description of Artificial Sequence: Synthetic polypeptide
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 78 - protein, 577 aa, synthetic construct
  source 1..577: note = Description of Artificial Sequence: Synthetic polypeptide
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 79 - protein, 613 aa, synthetic construct
  source 1..613: note = Description of Artificial Sequence: Synthetic polypeptide
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 80 - protein, 615 aa, synthetic construct
  source 1..615: note = Description of Artificial Sequence: Synthetic polypeptide
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 81 - protein, 775 aa, synthetic construct
  source 1..775: note = Description of Artificial Sequence: Synthetic polypeptide
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 82 - protein, 777 aa, synthetic construct
  source 1..777: note = Description of Artificial Sequence: Synthetic polypeptide
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 83 - protein, 610 aa, synthetic construct
  source 1..610: note = Description of Artificial Sequence: Synthetic polypeptide
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 84 - protein, 632 aa, synthetic construct
  source 1..632: note = Description of Artificial Sequence: Synthetic polypeptide
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 85 - protein, 625 aa, synthetic construct
  source 1..625: note = Description of Artificial Sequence: Synthetic polypeptide
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 86 - protein, 517 aa, synthetic construct
  source 1..517: note = Description of Artificial Sequence: Synthetic polypeptide
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 87 - protein, 410 aa, synthetic construct
  source 1..410: note = Description of Artificial Sequence: Synthetic polypeptide
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 88 - protein, 486 aa, synthetic construct
  source 1..486: note = Description of Artificial Sequence: Synthetic polypeptide
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 89 - protein, 602 aa, synthetic construct
  source 1..602: note = Description of Artificial Sequence: Synthetic polypeptide
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 90 - protein, 494 aa, synthetic construct
  source 1..494: note = Description of Artificial Sequence: Synthetic polypeptide
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 91 - protein, 526 aa, synthetic construct
  source 1..526: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 92 - protein, 649 aa, synthetic construct
  source 1..649: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 93 - protein, 414 aa, synthetic construct
  source 1..414: note = Description of Artificial Sequence: Synthetic polypeptide
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 94 - protein, 413 aa, synthetic construct
  source 1..413: note = Description of Artificial Sequence: Synthetic polypeptide
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 95 - protein, 449 aa, synthetic construct
  source 1..449: note = Description of Artificial Sequence: Synthetic polypeptide
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 96 - protein, 711 aa, synthetic construct
  source 1..711: note = Description of Artificial Sequence: Synthetic polypeptide
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 97 - protein, 574 aa, synthetic construct
  source 1..574: note = Description of Artificial Sequence: Synthetic polypeptide
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 98 - protein, 400 aa, synthetic construct
  source 1..400: note = Description of Artificial Sequence: Synthetic polypeptide
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 99 - protein, 743 aa, synthetic construct
  source 1..743: note = Description of Artificial Sequence: Synthetic polypeptide
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 100 - protein, 769 aa, synthetic construct
  source 1..769: note = Description of Artificial Sequence: Synthetic polypeptide
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 101 - protein, 666 aa, synthetic construct
  source 1..666: note = Description of Artificial Sequence: Synthetic polypeptide
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 102 - protein, 564 aa, synthetic construct
  source 1..564: note = Description of Artificial Sequence: Synthetic polypeptide
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 103 - protein, 565 aa, synthetic construct
  source 1..565: note = Description of Artificial Sequence: Synthetic polypeptide
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 104 - protein, 499 aa, synthetic construct
  source 1..499: note = Description of Artificial Sequence: Synthetic polypeptide
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 105 - protein, 560 aa, synthetic construct
  source 1..560: note = Description of Artificial Sequence: Synthetic polypeptide
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 106 - protein, 404 aa, synthetic construct
  source 1..404: note = Description of Artificial Sequence: Synthetic polypeptide
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 107 - protein, 392 aa, synthetic construct
  source 1..392: note = Description of Artificial Sequence: Synthetic polypeptide
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 108 - protein, 500 aa, synthetic construct
  source 1..500: note = Description of Artificial Sequence: Synthetic polypeptide
KEPLNIGKTAKAVFKEIDPTSLNRAANYDASIELNCKECKFKPFKNVKRYEFNFYNNWYRCNPNSCLQSTYKAQVRKVEIGYEKLKNEILTQMQYYPWFGRLYQNFFHDERDKMTSLDEIQVIGVQNKVFFNTVEKAWREIIKKRFKDNKETMETIPELKHAAGHGKRKLSNKSLLRRRFAFVQKSFKFVDNSDVSYRSFSNNIACVLPSRIGVDLGGVISRNPKREYIPQEISFNAFWKQHEGLKKGRNIEIQSVQYKGETVKRIEADTGEDKAWGKNRQRRFTSLILKLVPKQGGKKVWKYPEKRNEGNYEYFPIPIEFILDSGETSIRFGGDEGEAGKQKHLVIPFNDSKATPLASQQTLLENSRFNAEVKSCIGLAIYANYFYGYARNYVISSIYHKNSKNGQAITAIYLESIAHNYVKAIERQLQNLLLNLRDFSFMESHKKELKKYFGGDLEGTGGAQKRREKEEKIEKEIEQSYLPRLIRLSLTKMVTKQVEM

SEQ ID NO: 109 - protein, 507 aa, synthetic construct
  source 1..507: note = Description of Artificial Sequence: Synthetic polypeptide
ELIVNENKDPLNIGKTAKAVFKEIDPTSINRAANYDASIELACKECKFKPFNNTKRHDFSFYSNWHRCSPNSCLQSTYRAKIRKTEIGYEKLKNEILNQMQYYPWFGRLYQNFFNDQRDKMTSLDEIQVTGVQNKIFFNTVEKAWREIIKKRFRDNKETMRTIPDLKNKSGHGSRKLSNKSLLRRRFAFAQKSFKLVDNSDVSYRAFSNNVACVLPSKIGVDIGGIINKDLKREYIPQEITFNVFWKQHDGLKKGRNIEIHSVQYKGEIVKRIEADTGEDKAWGKNRQRRFTSLILKITPKQGGKKIWKFPEKKNASDYEYFPIPIEFILDNGDASIKFGGEEGEVGKQKHLLIPFNDSKATPLSSKQMLLETSRFNAEVKSTIGLALYANYFVSYARNYVIKSTYHKNSKKGQIVTEIYLESISQNFVRAIQRQLQSLMLNLKDWGFMQTHKKELKKYFGSDLEGSKGGQKRREKEEKIEKEIEASYLPRLIRLSLTKSVTKAEEM

SEQ ID NO: 110 - protein, 529 aa, synthetic construct
  source 1..529: note = Description of Artificial Sequence: Synthetic polypeptide
PEEKTSKLKPNSINLAANYDANEKFNCKECKFHPFKNKKRYEFNFYNNLHGCKSCTKSTNNPAVKRIEIGYQKLKFEIKNQMEAYPWFGRLRINFYSDEKRKMSELNEMQVTGVKNKIFFDAIECAWREILKKRFRESKETLITIPKLKNKAGHGARKHRNKKLLIRRRAFMKKNFHFLDNDSISYRSFANNIACVLPSKVGVDIGGIISPDVGKDIKPVDISLNLMWASKEGIKSGRKVEIYSTQYDGNMVKKIEAETGEDKSWGKNRKRRQTSLLLSIPKPSKQVQEFDFKEWPRYKDIEKKVQWRGFPIKIIFDSNHNSIEFGTYQGGKQKVLPIPFNDSKTTPLGSKMNKLEKLRFNSKIKSRLGSAIAANKFLEAARTYCVDSLYHEVSSANAIGKGKIFIEYYLEILSQNYIEAAQKQLQRFIESIEQWFVADPFQGRLKQYFKDDLKRAKCFLCANREVQTTCYAAVKLHKSCAEKVKDKNKELAIKERNNKEDAVIKEVEASNYPRVIRLKLTKTITNKAM

SEQ ID NO: 111 - protein, 726 aa, synthetic construct
  source 1..726: note = Description of Artificial Sequence: Synthetic polypeptide
SESENKIIEQYYAFLYSFRDKYEKPEFKNRGDIKRKLQNKWEDFLKEQNLKNDKKLSNYIFSNRNFRRSYDREEENEEGIDEKKSKPKRINCFEKEKNLKDQYDKDAINASANKDGAQKWGCFECIFFPMYKIESGDPNKRIIINKTRFKLFDFYLNLKGCKSCLRSTYHPYRSNVYIESNYDKLKREIGNFLQQKNIFQRMRKAKVSEGKYLTNLDEYRLSCVAMHFKNRWLFFDSIQKVLRETIKQRLKQMRESYDEQAKTKRSKGHGRAKYEDQVRMIRRRAYSAQAHKLLDNGYITLFDYDDKEINKVCLTAINQEGFDIGGYLNSDIDNVMPPIEISFHLKWKYNEPILNIESPFSKAKISDYLRKIREDLNLERGKEGKARSKKNVRRKVLASKGEDGYKKIFTDFFSKWKEELEGNAMERVLSQSSGDIQWSKKKRIHYTTLVLNINLLDKKGVGNLKYYEIAEKTKILSFDKNENKFWPITIQVLLDGYEIGTEYDEIKQLNEKTSKQFTIYDPNTKIIKIPFTDSKAVPLGMLGINIATLKTVKKTERDIKVSKIFKGGLNSKIVSKIGKGIYAGYFPTVDKEILEEVEEDTLDNEFSSKSQRNIFLKSIIKNYDKMLKEQLFDFYSFLVRNDLGVRFLTDRELQNIEDESFNLEKRFFETDRDRIARWFDNTNTDDGKEKFKKLANEIVDSYKPRLIRLPVVRVIKRIQPVKQREM

SEQ ID NO: 112 - protein, 517 aa, synthetic construct
  source 1..517: note = Description of Artificial Sequence: Synthetic polypeptide
KYSTRDFSELNEIQVTACKQDEFFKVIQNAWREIIKKRFLENRENFIEKKIFKNKKGRGKRQESDKTIQRNRASVMKNFQLIENEKIILRAPSGHVACVFPVKVGLDIGGFKTDDLEKNIFPPRTITINVFWKNRDRQRKGRKLEVWGIKARTKLIEKVHKWDKLEEVKKKRLKSLEQKQEKSLDNWSEVNNDSFYKVQIDELQEKIDKSLKGRTMNKILDNKAKESKEAEGLYIEWEKDFEGEMLRRIEASTGGEEKWGKRRQRRHTSLLLDIKNNSRGSKEIINFYSYAKQGKKEKKIEFFPFPLTITLDAEEESPLNIKSIPIEDKNATSKYFSIPFTETRATPLSILGDRVQKFKTKNISGAIKRNLGSSISSCKIVQNAETSAKSILSLPNVKEDNNMEIFINTMSKNYFRAMMKQMESFIFEMEPKTLIDPYKEKAIKWFEVAASSRAKRKLKKLSKADIKKSELLLSNTEEFEKEKQEKLEALEKEIEEFYLPRIVRLQLTKTILETPVM

SEQ ID NO: 113 - protein, 481 aa, synthetic construct
  source 1..481: note = Description of Artificial Sequence: Synthetic polypeptide
KKLQLLGHKILLKEYDPNAVNAAANFETSTAELCGQCKMKPFKNKRRFQYTFGKNYHGCLSCIQNVYYAKKRIVQIAKEELKHQLTDSIASIPYKYTSLFSNTNSIDELYILKQERAAFFSNTNSIDELYITGIENNIAFKVISAIWDEIIKKRRQRYAESLTDTGTVKANRGHGGTAYKSNTRQEKIRALQKQTLHMVTNPYISLARYKNNYIVATLPRTIGMHIGAIKDRDPQKKLSDYAINFNVFWSDDRQLIELSTVQYTGDMVRKIEAETGENNKWGENMKRTKTSLLLEILTKKTTDELTFKDWAFSTKKEIDSVTKKTYQGFPIGIIFEGNESSVKFGSQNYFPLPFDAKITPPTAEGFRLDWLRKGSFSSQMKTSYGLAIYSNKVTNAIPAYVIKNMFYKIARAENGKQIKAKFLKKYLDIAGNNYVPFIIMQHYRVLDTFEEMPISQPKVIRLSLTKTQHIIIKKDKTDSKM

SEQ ID NO: 114 - protein, 534 aa, synthetic construct
  source 1..534: note = Description of Artificial Sequence: Synthetic polypeptide
NTSNLINLGKKAINISANYDANLEVGCKNCKFLSSNGNFPRQTNVKEGCHSCEKSTYEPSIYLVKIGERKAKYDVLDSLKKFTFQSLKYQSKKSMKSRNKKPKELKEFVIFANKNKAFDVIQKSYNHLILQIKKEINRMNSKKRKKNHKRRLFRDREKQLNKLRLIESSNLFLPRENKGNNHVFTYVAIHSVGRDIGVIGSYDEKLNFETELTYQLYFNDDKRLLYAYKPKQNKIIKIKEKLWNLRKEKEPLDLEYEKPLNKSITFSIKNDNLFKVSKDLMLRRAKFNIQGKEKLSKEERKINRDLIKIKGLVNSMSYGRFDELKKEKNIWSPHIYREVRQKEIKPCLIKNGDRIEIFEQLKKKMERLRRFREKRQKKISKDLIFAERIAYNFHTKSIKNTSNKINIDQEAKRGKASYMRKRIGYETFKNKYCEQCLSKGNVYRNVQKGCSCFENPFDWIKKGDENLLPKKNEDLRVKGAFRDEALEKQIVKIAFNIAKGYEDFYDNLGESTEKDLKLKFKVGTTINEQESLKL

SEQ ID NO: 115 - protein, 537 aa, synthetic construct
  source 1..537: note = Description of Artificial Sequence: Synthetic polypeptide
TSNPIKLGKKAINISANYDSNLQIGCKNCKFLSYNGNFPRQTNVKEGCHSCEKSTYEPPVYTVRIGERRSKYDVLDSLKKFIFLSLKYRQSKKMKTRSKGIRGLEEFVISANLKKAMDVIQKSYRHLILNIKNEIVRMNGKKRNKNHKRLLFRDREKQLNKLRLIEGSSFFKPPTVKGDNSIFTCVAIHNIGRDIGIAGDYFDKLEPKIELTYQLYYEYNPKKESEINKRLLYAYKPKQNKIIEIKEKLWNLRKEKSPLDLEYEKPLTKSITFLVKRDGVFRISKDLMLRKAKFIIQGKEKLSKEERKINRDLIKIKSNIISLTYGRFDELKKDKTIWSPHIFRDVKQGKITPCIERKGDRMDIFQQLRKKSERLRENRKKRQKKISKDLIFAERIAYNFHTKSIKNTSNLINIKHEAKRGKASYMRKRIGNETFRIKYCEQCFPKNNVYKNVQKGCSCFEDPFEYIKKGNEDLIPNKNQDLKAKGAFRDDALEKQIIKVAFNIAKGYEDFYENLKKTTEKDIRLKFKVGTIISEEM

SEQ ID NO: 116 - protein, 541 aa, synthetic construct
  source 1..541: note = Description of Artificial Sequence: Synthetic polypeptide
NNSINLSKKAINISANYDANLQVRCKNCKFLSSNGNFPRQTDVKEGCHSCEKSTYEPPVYDVKIGEIKAKYEVLDSLKKFTFQSLKYQLSKSMKFRSKKIKELKEFVIFAKESKALNVINRSYKHLILNIKNDINRMNSKKRIKNHKGRLFLDRQKQLSKLKLIEGSSFFVPAKNVGNKSVFTCVAIHSIGRDIGIAGLYDSFTKPVNEITYQIFFSGERRLLYAYKPKQLKILSIKENLWSLKNEKKPLDLLYEKPLGKNLNFNVKGGDLFRVSKDLMIRNAKFNVHGRQRLSDEERLINRNFIKIKGEVVSLSYGRFEELKKDRKLWSPHIFKDVRQNKIKPCLVMQGQRIDIFEQLKRKLELLKKIRKSRQKKLSKDLIFGERIAYNFHTKSIKNTSNKINIDSDAKRGRASYMRKRIGNETFKLKYCDVCFPKANVYRRVQNGCSCSENPYNYIKKGDKDLLPKKDEGLAIKGAFRDEKLNKQIIKVAFNIAKGYEDFYDDLKKRTEKDVDLKFKIGTTVLDQKPMEIFDGIVITWL

SEQ ID NO: 117 - protein, 542 aa, synthetic construct
  source 1..542: note = Description of Artificial Sequence: Synthetic polypeptide
LLTTVVETNNLAKKAINVAANFDANIDRQYYRCTPNLCRFIAQSPRETKEKDAGCSSCTQSTYDPKVYVIKIGKLLAKYEILKSLKRFLFMNRYFKQKKTERAQQKQKIGTELNEMSIFAKATNAMEVIKRATKHCTYDIIPETKSLQMLKRRRHRVKVRSLLKILKERRMKIKKIPNTFIEIPKQAKKNKSDYYVAAALKSCGIDVGLCGAYEKNAEVEAEYTYQLYYEYKGNSSTKRILYCYNNPQKNIREFWEAFYIQGSKSHVNTPGTIRLKMEKFLSPITIESEALDFRVWNSDLKIRNGQYGFIKKRSLGKEAREIKKGMGDIKRKIGNLTYGKSPSELKSIHVYRTERENPKKPRAARKKEDNFMEIFEMQRKKDYEVNKKRRKEATDAAKIMDFAEEPIRHYHTNNLKAVRRIDMNEQVERKKTSVFLKRIMQNGYRGNYCRKCIKAPEGSNRDENVLEKNEGCLDCIGSEFIWKKSSKEKKGLWHTNRLLRRIRLQCFTTAKAYENFYNDLFEKKESSLDIIKLKVSITTKSM

SEQ ID NO: 118 - protein, 564 aa, synthetic construct
  source 1..564: note = Description of Artificial Sequence: Synthetic polypeptide
ASTMNLAKQAINFAANYDSNLEIGCKGCKFMSTWSKKSNPKFYPRQNNQANKCHSCTYSTGEPEVPIIEIGERAAKYKIFTALKKFVFMSVAYKERRRQRFKSKKPKELKELAICSNREKAMEVIQKSVVHCYGDVKQEIPRIRKIKVLKNHKGRLFYKQKRSKIKIAKLEKGSFFKTFIPKVHNNGCHSCHEASLNKPILVTTALNTIGADIGLINDYSTIAPTETDISWQVYYEFIPNGDSEAVKKRLLYFYKPKGALIKSIRDKYFKKGHENAVNTGFFKYQGKIVKGPIKFVNNELDFARKPDLKSMKIKRAGFAIPSAKRLSKEDREINRESIKIKNKIYSLSYGRKKTLSDKDIIKHLYRPVRQKGVKPLEYRKAPDGFLEFFYSLKRKERRLRKQKEKRQKDMSEIIDAADEFAWHRHTGSIKKTTNHINFKSEVKRGKVPIMKKRIANDSFNTRHCGKCVKQGNAINKYYIEKQKNCFDCNSIEFKWEKAALEKKGAFKLNKRLQYIVKACFNVAKAYESFYEDFRKGEEESLDLKFKIGTTTTLKQYPQNKARAM

SEQ ID NO: 119 - protein, 610 aa, synthetic construct
  source 1..610: note = Description of Artificial Sequence: Synthetic polypeptide
HSHNLMLTKLGKQAINFAANYDANLEIGCKNCKFLSYSPKQANPKKYPRQTDVHEDGNIACHSCMQSTKEPPVYIVPIGERKSKYEILTSLNKFTFLALKYKEKKRQAFRAKKPKELQELAIAFNKEKAIKVIDKSIQHLILNIKPEIARIQRQKRLKNRKGKLLYLHKRYAIKMGLIKNGKYFKVGSPKKDGKKLLVLCALNTIGRDIGIIGNIEENNRSETEITYQLYFDCLDANPNELRIKEIEYNRLKSYERKIKRLVYAYKPKQTKILEIRSKFFSKGHENKVNTGSFNFENPLNKSISIKVKNSAFDFKIGAPFIMLRNGKFHIPTKKRLSKEEREINRTLSKIKGRVFRLTYGRNISEQGSKSLHIYRKERQHPKLSLEIRKQPDSFIDEFEKLRLKQNFISKLKKQRQKKLADLLQFADRIAYNYHTSSLEKTSNFINYKPEVKRGRTSYIKKRIGNEGFEKLYCETCIKSNDKENAYAVEKEELCFVCKAKPFTWKKTNKDKLGIFKYPSRIKDFIRAAFTVAKSYNDFYENLKKKDLKNEIFLKFKIGLILSHEKKNHISIAKSVAEDERISGKSIKNILNKSIKLEKNCYSCFFHKEDM

SEQ ID NO: 120 - protein, 552 aa, synthetic construct
  source 1..552: note = Description of Artificial Sequence: Synthetic polypeptide
SLERVIDKRNLAKKAINIAANFDANINKGFYRCETNQCMFIAQKPRKTNNTGCSSCLQSTYDPVIYVVKVGEMLAKYEILKSLKRFVFMNRSFKQKKTEKAKQKERIGGELNEMSIFANAALAMGVIKRAIRHCHVDIRPEINRLSELKKTKHRVAAKSLVKIVKQRKTKWKGIPNSFIQIPQKARNKDADFYVASALKSGGIDIGLCGTYDKKPHADPRWTYQLYFDTEDESEKRLLYCYNDPQAKIRDFWKTFYERGNPSMVNSPGTIEFRMEGFFEKMTPISIESKDFDFRVWNKDLLIRRGLYEIKKRKNLNRKAREIKKAMGSVKRVLANMTYGKSPTDKKSIPVYRVEREKPKKPRAVRKEENELADKLENYRREDFLIRNRRKREATEIAKIIDAAEPPIRHYHTNHLRAVKRIDLSKPVARKNTSVFLKRIMQNGYRGNYCKKCIKGNIDPNKDECRLEDIKKCICCEGTQNIWAKKEKLYTGRINVLNKRIKQMKLECFNVAKAYENFYDNLAALKEGDLKVLKLKVSIPALNPEASDPEEDM

SEQ ID NO: 121 - protein, 534 aa, synthetic construct
  source 1..534: note = Description of Artificial Sequence: Synthetic polypeptide
NASINLGKRAINLSANYDSNLVIGCKNCKFLSFNGNFPRQTNVREGCHSCDKSTYAPEVYIVKIGERKAKYDVLDSLKKFTFQSLKYQIKKSMRERSKKPKELLEFVIFANKDKAFNVIQKSYEHLILNIKQEINRMNGKKRIKNHKKRLFKDREKQLNKLRLIGSSSLFFPRENKGDKDLFTYVAIHSVGRDIGVAGSYESHIEPISDLTYQLFINNEKRLLYAYKPKQNKIIELKENLWNLKKEKKPLDLEFTKPLEKSITFSVKNDKLFKVSKDLMLRQAKFNIQGKEKLSKEERQINRDFSKIKSNVISLSYGRFEELKKEKNIWSPHIYREVKQKEIKPCIVRKGDRIELFEQLKRKMDKLKKFRKERQKKISKDLNFAERIAYNFHTKSIKNTSNKINIDQEAKRGKASYMRKRIGNESFRKKYCEQCFSVGNVYHNVQNGCSCFDNPIELIKKGDEGLIPKGKEDRKYKGALRDDNLQMQIIRVAFNIAKGYEDFYNNLKEKTEKDLKLKFKIGTTISTQESNNKEM

SEQ ID NO: 122 - protein, 577 aa, synthetic construct
  source 1..577: note = Description of Artificial Sequence: Synthetic polypeptide
SNLIKLGKQAINFAANYDANLEVGCKNCKFLSSTNKYPRQTNVHLDNKMACRSCNQSTMEPAIYIVRIGEKKAKYDIYNSLTKFNFQSLKYKAKRSQRFKPKQPKELQELSIAVRKEKALDIIQKSIDHLIQDIRPEIPRIKQQKRYKNHVGKLFYLQKRRKNKLNLIGKGSFFKVFSPKEKKNELLVICALTNIGRDIGLIGNYNTIINPLFEVTYQLYYDYIPKKNNKNVQRRLLYAYKSKNEKILKLKEAFFKRGHENAVNLGSFSYEKPLEKSLTLKIKNDKDDFQVSPSLRIRTGRFFVPSKRNLSRQEREINRRLVKIKSKIKNMTYGKFETARDKQSVHIFRLERQKEKLPLQFRKDEKEFMEEFQKLKRRTNSLKKLRKSRQKKLADLLQLSEKVVYNNHTGTLKKTSNFLNFSSSVKRGKTAYIKELLGQEGFETLYCSNCINKGQKTRYNIETKEKCFSCKDVPFVWKKKSTDKDRKGAFLFPAKLKDVIKATFTVAKAYEDFYDNLKSIDEKKPYIKFKIGLILAHVRHEHKARAKEEAGQKNIYNKPIKIDKNCKECFFFKEEAM

SEQ ID NO: 123 - protein, 613 aa, synthetic construct
  source 1..613: note = Description of Artificial Sequence: Synthetic polypeptide
NTTRKKFRKRTGFPQSDNIKLAYCSAIVRAANLDADIQKKHNQCNPNLCVGIKSNEQSRKYEHSDRQALLCYACNQSTGAPKVDYIQIGEIGAKYKILQMVNAYDFLSLAYNLTKLRNGKSRGHQRMSQLDEVVIVADYEKATEVIKRSINHLLDDIRGQLSKLKKRTQNEHITEHKQSKIRRKLRKLSRLLKRRRWKWGTIPNPYLKNWVFTKKDPELVTVALLHKLGRDIGLVNRSKRRSKQKLLPKVGFQLYYKWESPSLNNIKKSKAKKLPKRLLIPYKNVKLFDNKQKLENAIKSLLESYQKTIKVEFDQFFQNRTEEIIAEEQQTLERGLLKQLEKKKNEFASQKKALKEEKKKIKEPRKAKLLMEESRSLGFLMANVSYALFNTTIEDLYKKSNVVSGCIPQEPVVVFPADIQNKGSLAKILFAPKDGFRIKFSGQHLTIRTAKFKIRGKEIKILTKTKREILKNIEKLRRVWYREQHYKLKLFGKEVSAKPRFLDKRKTSIERRDPNKLADQTDDRQAELRNKEYELRHKQHKMAERLDNIDTNAQNLQTLSFWVGEADKPPKLDEKDARGFGVRTCISAWKWFMEDLLKKQEEDPLLKLKLSIM

SEQ ID NO: 124 - protein, 615 aa, synthetic construct
  source 1..615: note = Description of Artificial Sequence: Synthetic polypeptide
PKKPKFQKRTGFPQPDNLRKEYCLAIVRAANLDADFEKKCTKCEGIKTNKKGNIVKGRTYNSADKDNLLCYACNISTGAPAVDYVFVGALEAKYKILQMVKAYDFHSLAYNLAKLWKGRGRGHQRMGGLNEVVIVSNNEKALDVIEKSLNHFHDEIRGELSRLKAKFQNEHLHVHKESKLRRKLRKISRLLKRRRWKWDVIPNSYLRNFTFTKTRPDFISVALLHRVGRDIGLVTKTKIPKPTDLLPQFGFQIYYTWDEPKLNKLKKSRLRSEPKRLLVPYKKIELYKNKSVLEEAIRHLAEVYTEDLTICFKDFFETQKRKFVSKEKESLKRELLKELTKLKKDFSERKTALKRDRKEIKEPKKAKLLMEESRSLGFLAANTSYALFNLIAADLYTKSKKACSTKLPRQLSTILPLEIKEHKSTTSLAIKPEEGFKIRFSNTHLSIRTPKFKMKGADIKALTKRKREILKNATKLEKSWYGLKHYKLKLYGKEVAAKPRFLDKRNPSIDRRDPKELMEQIENRRNEVKDLEYEIRKGQHQMAKRLDNVDTNAQNLQTKSFWVGEADKPPELDSMEAKKLGLRTCISAWKWFMKDLVLLQEKSPNLKLKLSLTEM

SEQ ID NO: 125 - protein, 775 aa, synthetic construct
  source 1..775: note = Description of Artificial Sequence: Synthetic polypeptide
KFSKRQEGFLIPDNIDLYKCLAIVRSANLDADVQGHKSCYGVKKNGTYRVKQNGKKGVKEKGRKYVFDLIAFKGNIEKIPHEAIEEKDQGRVIVLGKFNYKLILNIEKNHNDRASLEIKNKIKKLVQISSLETGEFLSDLLSGKIGIDEVYGIIEPDVFSGKELVCKACQQSTYAPLVEYMPVGELDAKYKILSAIKGYDFLSLAYNLSRNRANKKRGHQKLGGGELSEVVISANYDKALNVIKRSINHYHVEIKPEISKLKKKMQNEPLKVMKQARIRRELHQLSRKVKRLKWKWGMIPNPELQNIIFEKKEKDFVSYALLHTLGRDIGLFKDTSMLQVPNISDYGFQIYYSWEDPKLNSIKKIKDLPKRLLIPYKRLDFYIDTILVAKVIKNLIELYRKSYVYETFGEEYGYAKKAEDILFDWDSINLSEGIEQKIQKIKDEFSDLLYEARESKRQNFVESFENILGLYDKNFASDRNSYQEKIQSMIIKKQQENIEQKLKREFKEVIERGFEGMDQNKKYYKVLSPNIKGGLLYTDTNNLGFFRSHLAFMLLSKISDDLYRKNNLVSKGGNKGILDQTPETMLTLEFGKSNLPNISIKRKFFNIKYNSSWIGIRKPKFSIKGAVIREITKKVRDEQRLIKSLEGVWHKSTHFKRWGKPRFNLPRHPDREKNNDDNLMESITSRREQIQLLLREKQKQQEKMAGRLDKIDKEIQNLQTANFQIKQIDKKPALTEKSEGKQSVRNALSAWKWFMEDLIKYQKRTPILQLKLAKM

SEQ ID NO: 126 - protein, 777 aa, synthetic construct
  source 1..777: note = Description of Artificial Sequence: Synthetic polypeptide
KFSKRQEGFVIPENIGLYKCLAIVRSANLDADVQGHVSCYGVKKNGTYVLKQNGKKSIREKGRKYASDLVAFKGDIEKIPFEVIEEKKKEQSIVLGKFNYKLVLDVMKGEKDRASLTMKNKSKKLVQVSSLGTDEFLLTLLNEKFGIEEIYGIIEPEVFSGKKLVCKACQQSTYAPLVEYMPVGELDSKYKILSAIKGYDFLSLAYNLARHRSNKKRGHQKLGGGELSEVVISANNAKALNVIKRSLNHYYSEIKPEISKLRKKMQNEPLKVGKQARMRRELHQLSRKVKRLKWKWGKIPNLELQNITFKESDRDFISYALLHTLGRDIGMFNKTEIKMPSNILGYGFQIYYDWEEPKLNTIKKSKNTPKRILIPYKKLDFYNDSILVARAIKELVGLFQESYEWEIFGNEYNYAKEAEVELIKLDEESINGNVEKKLQRIKENFSNLLEKAREKKRQNFIESFESIARLYDESFTADRNEYQREIQSFIIEKQKQSIEKKLKNEFKKIVEKKFNEQEQGKKHYRVLNPTIINEFLPKDKNNLGFLRSKIAFILLSKISDDLYKKSNAVSKGGEKGIIKQQPETILDLEFSKSKLPSINIKKKLFNIKYTSSWLGIRKPKFNIKGAKIREITRRVRDVQRTLKSAESSWYASTHFRRWGFPRFNQPRHPDKEKKSDDRLIESITLLREQIQILLREKQKGQKEMAGRLDDVDKKIQNLQTANFQIKQTGDKPALTEKSAGKQSFRNALSAWKWFMENLLKYQNKTPDLKLKIARTVM

SEQ ID NO: 127 - protein, 610 aa, synthetic construct
  source 1..610: note = Description of Artificial Sequence: Synthetic polypeptide
KWIEPNNIDFNKCLAITRSANLDADVQGHKMCYGIKTNGTYKAIGKINKKHNTGIIEKRRTYVYDLIVTKEKNEKIVKKTDFMAIDEEIEFDEKKEKLLKKYIKAEVLGTGELIRKDLNDGEKFDDLCSIEEPQAFRRSELVCKACNQSTYASDIRYIPIGEIEAKYKILKAIKGYDFLSLKYNLGRLRDSKKRGHQKMGQGELKEFVICANKEKALDVIKRSLNHYLNEVKDEISRLNKKMQNEPLKVNDQARWRRELNQISRRLKRLKWKWGEIPNPELKNLIFKSSRPEFVSYALIHTLGRDIGLINETELKPNNIQEYGFQIYYKWEDPELNHIKKVKNIPKRFIIPYKNLDLFGKYTILSRAIEGILKLYSSSFQYKSFKDPNLFAKEGEKKITNEDFELGYDEKIKKIKDDFKSYKKALLEKKKNTLEDSLNSILSVYEQSLLTEQINNVKKWKEGLLKSKESIHKQKKIENIEDIISRIEELKNVEGWIRTKERDIVNKEETNLKREIKKELKDSYYEEVRKDFSDLKKGEESEKKPFREEPKPIVIKDYIKFDVLPGENSALGFFLSHLSFNLFDSIQYELFEKSRLSSSKHPQIPETILDL

SEQ ID NO: 128 - protein, 632 aa, synthetic construct
  source 1..632: note = Description of Artificial Sequence: Synthetic polypeptide
FRKFVKRSGAPQPDNLNKYKCIAIVRAANLDADIMSNESSNCVMCKGIKMNKRKTAKGAAKTTELGRVYAGQSGNLLCTACTKSTMGPLVDYVPIGRIRAKYTILRAVKEYDFLSLAYNLARTRVSKKGGRQKMHSLSELVIAAEYEIAWNIIKSSVIHYHQETKEEISGLRKKLQAEHIHKNKEARIRREMHQISRRIKRLKWKWHMIPNSELHNFLFKQQDPSFVAVALLHTLGRDIGMINKPKGSAKREFIPEYGFQIYYKWMNPKLNDINKQKYRKMPKRSLIPYKNLNVFGDRELIENAMHKLLKLYDENLEVKGSKFFKTRVVAISSKESEKLKRDLLWKGELAKIKKDFNADKNKMQELFKEVKEPKKANALMKQSRNMGFLLQNISYGALGLLANRMYEASAKQSKGDATKQPSIVIPLEMEFGNAFPKLLLRSGKFAMNVSSPWLTIRKPKFVIKGNKIKNITKLMKDEKAKLKRLETSYHRATHFRPTLRGSIDWDSPYFSSPKQPNTHRRSPDRLSADITEYRGRLKSVEAELREGQRAMAKKLDSVDMTASNLQTSNFQLEKGEDPRLTEIDEKGRSIRNCISSWKKFMEDLMKAQEANPVIKIKIALKDESSVLSEDSM

SEQ ID NO: 129 - protein, 625 aa, synthetic construct
  source 1..625: note = Description of Artificial Sequence: Synthetic polypeptide
KFHPENLNKSYCLAIVRAANLDADIQGHINCIGIKSNKSDRNYENKLESLQNVELLCKACTKSTYKPNINSVPVGEKKAKYSILSEIKKYDFNSLVYNLKKYRKGKSRGHQKLNELRELVITSEYKKALDVINKSVNHYLVNIKNKMSKLKKILQNEHIHVGTLARIRRERNRISRKLDHYRKKWKFVPNKILKNYVFKNQSPDFVSVALLHKLGRDIGLITKTAILQKSFPEYSLQLYYKYDTPKLNYLKKSKFKSLPKRILISYKYPKFDINSNYIEESIDKLLKLYEESPIYKNNSKIIEFFKKSEDNLIKSENDSLKRGIMKEFEKVTKNFSSKKKKLKEELKLKNEDKNSKMLAKVSRPIGFLKAYLSYMLFNIISNRIFEFSRKSSGRIPQLPSCIINLGNQFENFKNELQDSNIGSKKNYKYFCNLLLKSSGFNISYEEEHLSIKTPNFFINGRKLKEITSEKKKIRKENEQLIKQWKKLTFFKPSNLNGKKTSDKIRFKSPNNPDIERKSEDNIVENIAKVKYKLEDLLSEQRKEFNKLAKKHDGVDVEAQCLQTKSFWIDSNSPIKKSLEKKNEKVSVKKKMKAIRSCISAWKWFMADLIEAQKETPMIKLKLALM

SEQ ID NO: 130 - protein, 517 aa, synthetic construct
  source 1..517: note = Description of Artificial Sequence: Synthetic polypeptide
TTLVPSHLAGIEVMDETTSRNEDMIQKETSRSNEDENYLGVKNKCGINVHKSGRGSSKHEPNMPPEKSGEGQMPKQDSTEMQQRFDESVTGETQVSAGATASIKTDARANSGPRVGTARALIVKASNLDRDIKLGCKPCEYIRSELPMGKKNGCNHCEKSSDIASVPKVESGFRKAKYELVRRFESFAADSISRHLGKEQARTRGKRGKKDKKEQMGKVNLDEIAILKNESLIEYTENQILDARSNRIKEWLRSLRLRLRTRNKGLKKSKSIRRQLITLRRDYRKWIKPNPYRPDEDPNENSLRLHTKLGVDIGVQGGDNKRMNSDDYETSFSITWRDTATRKICFTKPKGLLPRHMKFKLRGYPELILYNEELRIQDSQKFPLVDWERIPIFKLRGVSLGKKKVKALNRITEAPRLVVAKRIQVNIESKKKKVLTRYVYNDKSINGRLVKAEDSNKDPLLEFKKQAEEINSDAKYYENQEIAKNYLWGCEGLHKNLLEEQTKNPYLAFKYGFLNIV

SEQ ID NO: 131 - protein, 410 aa, synthetic construct
  source 1..410: note = Description of Artificial Sequence: Synthetic polypeptide
LDFKRTCSQELVLLPEIEGLKLSGTQGVTSLAKKLINKAANVDRDESYGCHHCIHTRTSLSKPVKKDCNSCNQSTNHPAVPITLKGYKIAFYELWHRFTSWAVDSISKALHRNKVMGKVNLDEYAVVDNSHIVCYAVRKCYEKRQRSVRLHKRAYRCRAKHYNKSQPKVGRIYKKSKRRNARNLKKEAKRYFQPNEITNGSSDALFYKIGVDLGIAKGTPETEVKVDVSICFQVYYGDARRVLRVRKMDELQSFHLDYTGKLKLKGIGNKDTFTIAKRNESLKWGSTKYEVSRAHKKFKPFGKKGSVKRKCNDYFRSIASWSCEAASQRAQSNLKNAFPYQKALVKCYKNLDYKGVKKNDMWYRLCSNRIFRYSRIAEDIAQYQSDKGKAKFEFVILAQSVAEYDISAIM

SEQ ID NO: 132 - protein, 602 aa, synthetic construct
  source 1..602: note = Description of Artificial Sequence: Synthetic polypeptide
VFLTDDKRKTALRKIRSAFRKTAEIALVRAQEADSLDRQAKKLTIETVSFGAPGAKNAFIGSLQGYNWNSHRANVPSSGSAKDVFRITELGLGIPQSAHEASIGKSFELVGNVVRYTANLLSKGYKKGAVNKGAKQQREIKGKEQLSFDLISNGPISGDKLINGQKDALAWWLIDKMGFHIGLAMEPLSSPNTYGITLQAFWKRHTAPRRYSRGVIRQWQLPFGRQLAPLIHNFFRKKGASIPIVLTNASKKLAGKGVLLEQTALVDPKKWWQVKEQVTGPLSNIWERSVPLVLYTATFTHKHGAAHKRPLTLKVIRISSGSVFLLPLSKVTPGKLVRAWMPDINILRDGRPDEAAYKGPDLIRARERSFPLAYTCVTQIADEWQKRALESNRDSITPLEAKLVTGSDLLQIHSTVQQAVEQGIGGRISSPIQELLAKDALQLVLQQLFMTVDLLRIQWQLKQEVADGNTSEKAVGWAIRISNIHKDAYKTAIEPCTSALKQAWNPLSGFEERTFQLDASIVRKRSTAKTPDDELVIVLRQQAAEMTVAVTQSVSKELMELAVRHSATLHLLVGEVASKQLSRSADKDRGAMDHWKLLSQSM

SEQ ID NO: 133 - protein, 494 aa, synthetic construct
  source 1..494: note = Description of Artificial Sequence: Synthetic polypeptide
EDLLQKALNTATNVAAIERHSCISCLFTESEIDVKYKTPDKIGQNTAGCQSCTFRVGYSGNSHTLPMGNRIALDKLRETIQRYAWHSLLFNVPPAPTSKRVRAISELRVAAGRERLFTVITFVQTNILSKLQKRYAANWTPKSQERLSRLREEGQHILSLLESGSWQQKEVVREDQDLIVCSALTKPGLSIGAFCRPKYLKPAKHALVLRLIFVEQWPGQIWGQSKRTRRMRRRKDVERVYDISVQAWALKGKETRISECIDTMRRHQQAYIGVLPFLILSGSTVRGKGDCPILKEITRMRYCPNNEGLIPLGIFYRGSANKLLRVVKGSSFTLPMWQNIETLPHPEPFSPEGWTATGALYEKNLAYWSALNEAVDWYTGQILSSGLQYPNQNEFLARLQNVIDSIPRKWFRPQGLKNLKPNGQEDIVPNEFVIPQNAIRAHHVIEWYHKTNDLVAKTLLGWGSQTTLNQTRPQGDLRFTYTRYYFREKEVPEV

SEQ ID NO: 134 - protein, 649 aa, synthetic construct
  source 1..649: note = Description of Artificial Sequence: Synthetic polypeptide
VPKKKLMRELAKKAVFEAIFNDPIPGSFGCKRCTLIDGARVTDAIEKKQGAKRCAGCEPCTFHTLYDSVKHALPAATGCDRTAIDTGLWEILTALRSYNWMSFRRNAVSDASQKQVWSIEELAIWADKERALRVILSALTHTIGKLKNGFSRDGVWKGGKQLYENLAQKDLAKGLFANGEIFGKELVEADHDMLAWTIVPNHQFHIGLIRGNWKPAAVEASTAFDARWLTNGAPLRDTRTHGHRGRRFNRTEKLTVLCIKRDGGVSEEFRQERDYELSVMLLQPKNKLKPEPKGELNSFEDLHDHWWFLKGDEATALVGLTSDPTVGDFIQLGLYIRNPIKAHGETKRRLLICFEPPIKLPLRRAFPSEAFKTWEPTINVFRNGRRDTEAYYDIDRARVFEFPETRVSLEHLSKQWEVLRLEPDRENTDPYEAQQNEGAELQVYSLLQEAAQKMAPKVVIDPFGQFPLELFSTFVAQLFNAPLSDTKAKIGKPLDSGFVVESHLHLLEEDFAYRDFVRVTFMGTEPTFRVIHYSNGEGYWKKTVLKGKNNIRTALIPEGAKAAVDAYKNKRCPLTLEAAILNEEKDRRLVLGNKALSLLAQTARGNLTILEALAAEVLRPLSGTEGVVHLHACVTRHSTLTESTETDNM

SEQ ID NO: 135 - protein, 414 aa, synthetic construct
  source 1..414: note = Description of Artificial Sequence: Synthetic polypeptide
VEKLFSERLKRAMWLKNEAGRAPPAETLTLKHKRVSGGHEKVKEELQRVLRSLSGTNQAAWNLGLSGGREPKSSDALKGEKSRVVLETVVFHSGHNRVLYDVIEREDQVHQRSSIMHMRRKGSNLLRLWGRSGKVRRKMREEVAEIKPVWHKDSRWLAIVEEGRQSVVGISSAGLAVFAVQESQCTTAEPKPLEYVVSIWFRGSKALNPQDRYLEFKKLKTTEALRGQQYDPIPFSLKRGAGCSLAIRGEGIKFGSRGPIKQFFGSDRSRPSHADYDGKRRLSLFSKYAGDLADLTEEQWNRTVSAFAEDEVRRATLANIQDFLSISHEKYAERLKKRIESIEEPVSASKLEAYLSAIFETFVQQREALASNFLMRLVESVALLISLEEKSPRVEFRVARYLAESKEGFNRKAM

SEQ ID NO: 136 - protein, 413 aa, synthetic construct
  source 1..413: note = Description of Artificial Sequence: Synthetic polypeptide
VVITQSELYKERLLRVMEIKNDRGRKEPRESQGLVLRFTQVTGGQEKVKQKLWLIFEGFSGTNQASWNFGQPAGGRKPNSGDALKGPKSRVTYETVVFHFGLRLLSAVIERHNLKQQRQTMAYMKRRAAARKKWARSGKKCSRMRNEVEKIKPKWHKDPRWFDIVKEGEPSIVGISSAGFAIYIVEEPNFPRQDPLEIEYAISIWFRRDRSQYLTFKKIQKAEKLKELQYNPIPFRLKQEKTSLVFESGDIKFGSRGSIEHFRDEARGKPPKADMDNNRRLTMFSVFSGNLTNLTEEQYARPVSGLLAPDEKRMPTLLKKLQDFFTPIHEKYGERIKQRLANSEASKRPFKKLEEYLPAIYLEFRARREGLASNWVLVLINSVRTLVRIKSEDPYIEFKVSQYLLEKEDNKAL

SEQ ID NO: 137 - protein, 449 aa, synthetic construct
  source 1..449: note = Description of Artificial Sequence: Synthetic polypeptide
KQDALFEERLKKAIFIKRQADPLQREELSLLPPNRKIVTGGHESAKDTLKQILRAINGTNQASWNPGTPSGKRDSKSADALAGPKSRVKLETVVFHVGHRLLKKVVEYQGHQKQQHGLKAFMRTCAAMRKKWKRSGKVVGELREQLANIQPKWHYDSRPLNLCFEGKPSVVGLRSAGIALYTIQKSVVPVKEPKPIEYAVSIWFRGPKAMDREDRCLEFKKLKIATELRKLQFEPIVSTLTQGIKGFSLYIQGNSVKFGSRGPIKYFSNESVRQRPPKADPDGNKRLALFSKFSGDLSDLTEEQWNRPILAFEGIIRRATLGNIQDYLTVGHEQFAISLEQLLSEKESVLQMSIEQQRLKKNLGKKAENEWVESFGAEQARKKAQGIREYISGFFQEYCSQREQWAENWVQQLNKSVRLFLTIQDSTPFIEFRVARYLPKGEKKKGKAM

SEQ ID NO: 138 - protein, 711 aa, synthetic construct
  source 1..711: note = Description of Artificial Sequence: Synthetic polypeptide
ANHAERHKRLRKEANRAANRNRPLVADCDTGDPLVGICRLLRRGDKMQPNKTGCRSCEQVEPELRDAILVSGPGRLDNYKYELFQRGRAMAVHRLLKRVPKLNRPKKAAGNDEKKAENKKSEIQKEKQKQRRMMPAVSMKQVSVADFKHVIENTVRHLFGDRRDREIAECAALRAASKYFLKSRRVRPRKLPKLANPDHGKELKGLRLREKRAKLKKEKEKQAELARSNQKGAVLHVATLKKDAPPMPYEKTQGRNDYTTFVISAAIKVGATRGTKPLLTPQPREWQCSLYWRDGQRWIRGGLLGLQAGIVLGPKLNRELLEAVLQRPIECRMSGCGNPLQVRGAAVDFFMTTNPFYVSGAAYAQKKFKPFGTKRASEDGAAAKAREKLMTQLAKVLDKVVTQAAHSPLDGIWETRPEAKLRAMIMALEHEWIFLRPGPCHNAAEEVIKCDCTGGHAILWALIDEARGALEHKEFYAVTRAHTHDCEKQKLGGRLAGFLDLLIAQDVPLDDAPAARKIKTLLEATPPAPCYKAATSIATCDCEGKFDKLWAIIDATRAGHGTEDLWARTLAYPQNVNCKCKAGKDLTHRLADFLGLLIKRDGPFRERPPHKVTGDRKLVFSGDKKCKGHQYVILAKAHNEEVVRAWISRWGLKSRTNKAGYAATELNLLLNWLSICRRRWMDMLTVQRDTPYIRMKTGRLVVDDKKERKAM

SEQ ID NO: 139 - protein, 574 aa, synthetic construct
  source 1..574: note = Description of Artificial Sequence: Synthetic polypeptide
AKQREALRVALERGIVRASNRTYTLVTNCTKGGPLPEQCRMIERGKARAMKWEPKLVGCGSCAAATVDLPAIEEYAQPGRLDVAKYKLTTQILAMATRRMMVRAAKLSRRKGQWPAKVQEEKEEPPEPKKMLKAVEMRPVAIVDFNRVIQTTIEHLWAERANADEAELKALKAAAAYFGPSLKIRARGPPKAAIGRELKKAHRKKAYAERKKARRKRAELARSQARGAAAHAAIRERDIPPMAYERTQGRNDVTTIPIAAAIKIAATRGARPLPAPKPMKWQCSLYWNEGQRWIRGGMLTAQAYAHAANIHRPMRCEMWGVGNPLKVRAFEGRVADPDGAKGRKAEFRLQTNAFYVSGAAYRNKKFKPFGTDRGGIGSARKKRERLMAQLAKILDKVVSQAAHSPLDDIWHTRPAQKLRAMIKQLEHEWMFLRPQAPTVEGTKPDVDVAGNMQRQIKALMAPDLPPIEKGSPAKRFTGDKRKKGERAVRVAEAHSDEVVTAWISRWGIQTRRNEGSYAAQELELLLNWLQICRRRWLDMTAAQRVSPYIRMKSGRMITDAADEGVAPIPLVENM

SEQ ID NO: 140 - protein, 743 aa, synthetic construct
  source 1..743: note = Description of Artificial Sequence: Synthetic polypeptide
KSISGRSIKHMACLKDMLKSEITEIEEKQKKESLRKWDYYSKFSDEILFRRNLNVSANHDANACYGCNPCAFLKEVYGFRIERRNNERIISYRRGLAGCKSCVQSTGYPPIEFVRRKFGADKAMEIVREVLHRRNWGALARNIGREKEADPILGELNELLLVDARPYFGNKSAANETNLAFNVITRAAKKFRDEGMYDIHKQLDIHSEEGKVPKGRKSRLIRIERKHKAIHGLDPGETWRYPHCGKGEKYGVWLNRSRLIHIKGNEYRCLTAFGTTGRRMSLDVACSVLGHPLVKKKRKKGKKTVDGTELWQIKKATETLPEDPIDCTFYLYAAKPTKDPFILKVGSLKAPRWKKLHKDFFEYSDTEKTQGQEKGKRVVRRGKVPRILSLRPDAKFKVSIWDDPYNGKNKEGTLLRMELSGLDGAKKPLILKRYGEPNTKPKNFVFWRPHITPHPLTFTPKHDFGDPNKKTKRRRVFNREYYGHLNDLAKMEPNAKFFEDREVSNKKNPKAKNIRIQAKESLPNIVAKNGRWAAFDPNDSLWKLYLHWRGRRKTIKGGISQEFQEFKERLDLYKKHEDESEWKEKEKLWENHEKEWKKTLEIHGSIAEVSQRCVMQSMMGPLDGLVQKKDYVHIGQSSLKAADDAWTFSANRYKKATGPKWGKISVSNLLYDANQANAELISQSISKYLSKQKDNQGCEGRKMKFLIKIIEPLRENFVKHTRWLHEMTQKDCEVRAQFSRVSM

SEQ ID NO: 141 - protein, 769 aa, synthetic construct
  source 1..769: note = Description of Artificial Sequence: Synthetic polypeptide
FPSDVGADALKHVRMLQPRLTDEVRKVALTRAPSDRPALARFAAVAQDGLAFVRHLNVSANHDSNCTFPRDPRDPRRGPCEPNPCAFLREVWGFRIVARGNERALSYRRGLAGCKSCVQSTGFPSVPFHRIGADDCMRKLHEILKARNWRLLARNIGREREADPLLTELSEYLLVDARTYPDGAAPNSGRLAENVIKRAAKKFRDEGMRDIHAQLRVHSREGKVPKGRLQRLRRIERKHRAIHALDPGPSWEAEGSARAEVQGVAVYRSQLLRVGHHTQQIEPVGIVARTLFGVGRTDLDVAVSVLGAPLTKRKKGSKTLESTEDFRIAKARETRAEDKIEVAFVLYPTASLLRDEIPKDAFPAMRIDRFLLKVGSVQADREILLQDDYYRFGDAEVKAGKNKGRTVTRPVKVPRLQALRPDAKFRVNVWADPFGAGDSPGTLLRLEVSGVTRRSQPLRLLRYGQPSTQPANFLCWRPHRVPDPMTFTPRQKFGERRKNRRTRRPRVFERLYQVHIKHLAHLEPNRKWFEEARVSAQKWAKARAIRRKGAEDIPVVAPPAKRRWAALQPNAELWDLYAHDREARKRFRGGRAAEGEEFKPRLNLYLAHEPEAEWESKRDRWERYEKKWTAVLEEHSRMCAVADRTLPQFLSDPLGARMDDKDYAFVGKSALAVAEAFVEEGTVERAQGNCSITAKKKFASNASRKRLSVANLLDVSDKADRALVFQAVRQYVQRQAENGGVEGRRMAFLRKLLAPLRQNFVCHTRWLHM

SEQ ID NO: 142 - protein, 564 aa, synthetic construct
  source 1..564: note = Description of Artificial Sequence: Synthetic polypeptide
AARKKKRGKIGITVKAKEKSPPAAGPFMARKLVNVAANVDGVEVHLCVECEADAHGSASARLLGGCRSCTGSIGAEGRLMGSVDVDRERVIAEPVHTETERLGPDVKAFEAGTAESKYAIQRGLEYWGVDLISRNRARTVRKMEEADRPESSTMEKTSWDEIAIKTYSQAYHASENHLFWERQRRVRQHALALFRRARERNRGESPLQSTQRPAPLVLAALHAEAAAISGRARAEYVLRGPSANVRAAAADIDAKPLGHYKTPSPKVARGFPVKRDLLRARHRIVGLSRAYFKPSDVVRGTSDAIAHVAGRNIGVAGGKPKEIEKTFTLPFVAYWEDVDRVVHCSSFKADGPWVRDQRIKIRGVSSAVGTFSLYGLDVAWSKPTSFYIRCSDIRKKFHPKGFGPMKHWRQWAKELDRLTEQRASCVVRALQDDEELLQTMERGQRYYDVFSCAATHATRGEADPSGGCSRCELVSCGVAHKVTKKAKGDTGIEAVAVAGCSLCESKLVGPSKPRVHRQMAALRQSHALNYLRRLQREWEALEAVQAPTPYLRFKYARHLEVRSM

SEQ ID NO: 143 - protein, 565 aa, synthetic construct
  source 1..565: note = Description of Artificial Sequence: Synthetic polypeptide
AAKKKKQRGKIGISVKPKEGSAPPADGPFMARKLVNVAANVDGVEVNLCIECEADAHGSAPARLLGGCKSCTGSIGAEGRLMGSVDVDRADAIAKPVNTETEKLGPDVQAFEAGTAETKYALQRGLEYWGVDLISRNRSRTVRRTEEGQPESATMEKTSWDEIAIKSYTRAYHASENHLFWERQRRVRQHALALFKRAKERNRGDSTLPREPGHGLVAIAALACEAYAVGGRNLAETVVRGPTFGTARAVRDVEIASLGRYKTPSPKVAHGSPVKRDFLRARHRIVGLARAYYRPSDVVRGTSDAIAHVAGRNIGVAGGKPRAVEAVFTLPFVAYWEDVDRVVHCSSFQVSAPWNRDQRMKIAGVTTAAGTFSLHGGELKWAKPTSFYIRCSDTRRKFRPKGFGPMKRWRQWAKDLDRLVEQRASCVVRALQDDAALLETMERGQRYYDVFACAVTHATRGEADRLAGCSRCALTPCQEAHRVTTKPRGDAGVEQVQTSDCSLCEGKLVGPSKPRLHRTLTLLRQEHGLNYLRRLQREWESLEAVQVPTPYLRFKYARHLEVRSM

SEQ ID NO: 144 - protein, 499 aa, synthetic construct
  source 1..499: note = Description of Artificial Sequence: Synthetic polypeptide
TDSQSESVPEVVYALTGGEVPGRVPPDGGSAEGARNAPTGLRKQRGKIKISAKPSKPGSPASSLARTLVNEAANVDGVQSSGCATCRMRANGSAPRALPIGCVACASSIGRAPQEETVCALPTTQGPDVRLLEGGHALRKYDIQRALEYWGVDLIGRNLDRQAGRGMEPAEGATATMKRVSMDELAVLDFGKSYYASEQHLFAARQRRVRQHAKALKIRAKHANRSGSVKRALDRSRKQVTALAREFFKPSDVVRGDSDALAHVVGRNLGVSRHPAREIPQTFTLPLCAYWEDVDRVISCSSLLAGEPFARDQEIRIEGVSSALGSLRLYRGAIEWHKPTSLYIRCSDTRRKFRPRGGLKKRWRQWAKDLDRLVEQRACCIVRSLQADVELLQTMERAQRFYDVHDCAATHVGPVAVRCSPCAGKQFDWDRYRLLAALRQEHALNYLRRLQREWESLEAQQVKMPYLRFKYARKLEVSGPLIGLEVRREPSMGTAIAEM

SEQ ID NO: 145 - protein, 358 aa, synthetic construct
  source 1..358: note = Description of Artificial Sequence: Synthetic polypeptide
AGTAGRRHGSLGARRSINIAGVTDRHGRWGCESCVYTRDQAGNRARCAPCDQSTYAPDVQEVTIGQRQAKYTIFLTLQSFSWTNTMRNNKRAAAGRSKRTTGKRIGQLAEIKITGVGLAHAHNVIQRSLQHNITKMWRAEKGKSKRVARLKKAKQLTKRRAYFRRRMSRQSRGNGFFRTGKGGIHAVAPVKIGLDVGMIASGSSEPADEQTVTLDAIWKGRKKKIRLIGAKGELAVAACRFREQQTKGDKCIPLILQDGEVRWNQNNWQCHPKKLVPLCGLEVSRKFVSQADRLAQNKVASPLAARFDKTSVKGTLVESDFAAVLVNVTSIYQQCHAMLLRSQEPTPSLRVQRTITSM

SEQ ID NO: 146 - protein, 369 aa, synthetic construct
  source 1..369: note = Description of Artificial Sequence: Synthetic polypeptide
GVRFSPAQSQVFFRTVIPQSVEARFAINMAAIHDAAGAFGCSVCRFEDRTPRNAKAVHGCSPCTRSTNRPDVFVLPVGAIKAKYDVFMRLLGFNWTHLNRRQAKRVTVRDRIGQLDELAISMLTGKAKAVLKKSICHNVDKSFKAMRGSLKKLHRKASKTGKSQLRAKLSDLRERTNTTQEGSHVEGDSDVALNKIGLDVGLVGKPDYPSEESVEVVVCLYFVGKVLILDAQGRIRDMRAKQYDGFKIPIIQRGQLTVLSVKDLGKWSLVRQDYVLAGDLRFEPKISKDRKYAECVKRIALITLQASLGFKERIPYYVTKQVEIKNASHIAFVTEAIQNCAENFREMTEYLMKYQEKSPDLKVLLTQLM

SEQ ID NO: 147 - protein, 486 aa, synthetic construct
  source 1..486: note = Description of Artificial Sequence: Synthetic polypeptide
RAVVGKVFLEQARRALNLATNFGTNHRTGCNGCYVTPGKLSIPQDGEKNAAGCTSCLMKATASYVSYPKPLGEKVAKYSTLDALKGFPWYSLRLNLRPNYRGKPINGVQEVAPVSKFRLAEEVIQAVQRYHFTELEQSFPGGRRRLRELRAFYTKEYRRAPEQRQHVVNGDRNIVVVTVLHELGFSVGMFNEVELLPKTPIECAVNVFIRGNRVLLEVRKPQFDKERLLVESLWKKDSRRHTAKWTPPNNEGRIFTAEGWKDFQLPLLLGSTSRSLRAIEKEGFVQLAPGRDPDYNNTIDEQHSGRPFLPLYLYLQGTISQEYCVFAGTWVIPFQDGISPYSTKDTFQPDLKRKAYSLLLDAVKHRLGNKVASGLQYGRFPAIEELKRLVRMHGATRKIPRGEKDLLKKGDPDTPEWWLLEQYPEFWRLCDAAAKRVSQNVGLLLSLKKQPLWQRRWLESRTRNEPLDNLPLSMALTLHLTNEEAL

SEQ ID NO: 148 - protein, 400 aa, synthetic construct
  source 1..400: note = Description of Artificial Sequence: Synthetic polypeptide
AAVYSKFYIENHFKMGIPETLSRIRGPSIIQGFSVNENYINIAGVGDRDFIFGCKKCKYTRGKPSSKKINKCHPCKRSTYPEPVIDVRGSISEFKYKIYNKLKQEPNQSIKQNTKGRMNPSDHTSSNDGIIINGIDNRIAYNVIFSSYKHLMEKQINLLRDTTKRKARQIKKYNNSGKKKHSLRSQTKGNLKNRYHMLGMFKKGSLTITNEGDFITAVRKVGLDISLYKNESLNKQEVETELCLNIKWGRTKSYTVSGYIPLPINIDWKLYLFEKETGLTLRLFGNKYKIQSKKFLIAQLFKPKRPPCADPVVKKAQKWSALNAHVQQMAGLFSDSHLLKRELKNRMHKQLDFKSLWVGTEDYIKWFEELSRSYVEGAEKSLEFFRQDYFCFNYTKQTTM

SEQ ID NO: 149 - protein, 666 aa, synthetic construct
  source 1..666: note = Description of Artificial Sequence: Synthetic polypeptide
PQQQRDLMLMAANYDQDYGNGCGPCTVVASAAYRPDPQAQHGCKRHLRTLGASAVTHVGLGDRTATITALHRLRGPAALAARARAAQAASAPMTPDTDAPDDRRRLEAIDADDVVLVGAHRALWSAVRRWADDRRAALRRRLHSEREWLLKDQIRWAELYTLIEASGTPPQGRWRNTLGALRGQSRWRRVLAPTMRATCAETHAELWDALAELVPEMAKDRRGLLRPPVEADALWRAPMIVEGWRGGHSVVVDAVAPPLDLPQPCAWTAVRLSGDPRQRWGLHLAVPPLGQVQPPDPLKATLAVSMRHRGGVRVRTLQAMAVDADAPMQRHLQVPLTLQRGGGLQWGIHSRGVRRREARSMASWEGPPIWTGLQLVNRWKGQGSALLAPDRPPDTPPYAPDAAVAPAQPDTKRARRTLKEACTVCRCAPGHMRQLQVTLTGDGTWRRFRLRAPQGAKRKAEVLKVATQHDERIANYTAWYLKRPEHAAGCDTCDGDSRLDGACRGCRPLLVGDQCFRRYLDKIEADRDDGLAQIKPKAQEAVAAMAAKRDARAQKVAARAAKLSEATGQRTAATRDASHEARAQKELEAVATEGTTVRHDAAAVSAFGSWVARKGDEYRHQVGVLANRLEHGLRLQELMAPDSVVADQQRASGHARVGYRYVLTAM

SEQ ID NO: 150 - protein, 560 aa, synthetic construct
  source 1..560: note = Description of Artificial Sequence: Synthetic polypeptide
AVAHPVGRGNAGSPGARGPEELPRQLVNRASNVTRPATYGCAPCRHVRLSIPKPVLTGCRACEQTTHPAPKRAVRGGADAAKYDLAAFFAGWAADLEGRNRRRQVHAPLDPQPDPNHEPAVTLQKIDLAEVSIEEFQRVLARSVKHRHDGRASREREKARAYAQVAKKRRNSHAHGARTRRAVRRQTRAVRRAHRMGANSGEILVASGAEDPVPEAIDHAAQLRRRIRACARDLEGLRHLSRRYLKTLEKPCRRPRAPDLGRARCHALVESLQAAERELEELRRCDSPDTAMRRLDAVLAAAASTDATFATGWTVVGMDLGVAPRGSAAPEVSPMEMAISVFWRKGSRRVIVSKPIAGMPIRRHELIRLEGLGTLRLDGNHYTGAGVTKGRGLSEGTEPDFREKSPSTLGFTLSDYRHESRWRPYGAKQGKTARQFFAAMSRELRALVEHQVLAPMGPPLLEAHERRFETLLKGQDNKSIHAGGGGRYVWRGPPDSKKRPAADGDWFRFGRGHADHRGWANKRHELAANYLQSAFRLWSTLAEAQEPTPYARYKYTRVTM

SEQ ID NO: 151 - protein, 404 aa, synthetic construct
  source 1..404: note = Description of Artificial Sequence: Synthetic polypeptide
WDFLTLQVYERHTSPEVCVAGNSTKCASGTRKSDHTHGVGVKLGAQEINVSANDDRDHEVGCNICVISRVSLDIKGWRYGCESCVQSTPEWRSIVRFDRNHKEAKGECLSRFEYWGAQSIARSLKRNKLMGGVNLDELAIVQNENVVKTSLKHLFDKRKDRIQANLKAVKVRMRERRKSGRQRKALRRQCRKLKRYLRSYDPSDIKEGNSCSAFTKLGLDIGISPNKPPKIEPKVEVVFSLFYQGACDKIVTVSSPESPLPRSWKIKIDGIRALYVKSTKVKFGGRTFRAGQRNNRRKVRPPNVKKGKRKGSRSQFFNKFAVGLDAVSQQLPIASVQGLWGRAETKKAQTICLKQLESNKPLKESQRCLFLADNWVVRVCGFLRALSQRQGPTPYIRYRYRCNM

SEQ ID NO: 152 - protein, 392 aa, synthetic construct
  source 1..392: note = Description of Artificial Sequence: Synthetic polypeptide
ARNVGQRNASRQSKRESAKARSRRVTGGHASVTQGVALINAAANADRDHTTGCEPCTWERVNLPLQEVIHGCDSCTKSSPFWRDIKVVNKGYREAKEEIMRIASGISADHLSRALSHNKVMGRLNLDEVCILDFRTVLDTSLKHLTDSRSNGIKEHIRAVHRKIRMRRKSGKTARALRKQYFALRRQWKAGHKPNSIREGNSLTALRAVGFDVGVSEGTEPMPAPQTEVVLSVFYKGSATRILRISSPHPIAKRSWKVKIAGIKALKLIRREHDFSFGRETYNASQRAEKRKFSPHAARKDFFNSFAVQLDRLAQQLCVSSVENLWVTEPQQKLLTLAKDTAPYGIREGARFADTRARLAWNWVFRVCGFTRALHQEQEPTPYCRFTWRSKM

SEQ ID NO: 153 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 154 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 155 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 156 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 157 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 158 - protein, 1285 aa, unidentified
  source 1..1285: note = Description of Unknown: Herbinix hemicellulosilytica sequence
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 159 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 160 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 161 - protein, 1224 aa, Bergeyella zoohelcum
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 162 - protein, 1126 aa, Prevotella intermedia
MEDDKKTTDSIRYELKDKHFWAAFLNLARHNVYITVNHINKILEEGEINRDGYETTLKNTWNEIKDINKKDRLSKLIIKHFPFLEAATYRLNPTDTTKQKEEKQAEAQSLESLRKSFFVFIYKLRDLRNHYSHYKHSKSLERPKFEEGLLEKMYNIFNASIRLVKEDYQYNKDINPDEDFKHLDRTEEEFNYYFTKDNEGNITESGLLFFVSLFLEKKDAIWMQQKLRGFKDNRENKKKMTNEVFCRSRMLLPKLRLQSTQTQDWILLDMLNELIRCPKSLYERLREEDREKFRVPIEIADEDYDAEQEPFKNTLVRHQDRFPYFALRYFDYNEIFTNLRFQIDLGTYHFSIYKKQIGDYKESHHLTHKLYGFERIQEFTKQNRPDEWRKFVKTFNSFETSKEPYIPETTPHYHLENQKIGIRFRNDNDKIWPSLKTNSEKNEKSKYKLDKSFQAEAFLSVHELLPMMFYYLLLKTENTDNDNEIETKKKENKNDKQEKHKIEEIIENKITEIYALYDTFANGEIKSIDELEEYCKGKDIEIGHLPKQMIAILKDEHKVMATEAERKQEEMLVDVQKSLESLDNQINEEIENVERKNSSLKSGKIASWLVNDMMRFQPVQKDNEGKPLNNSKANSTEYQLLQRTLAFFGSEHERLAPYFKQTKLIESSNPHPFLKDTEWEKCNNILSFYRSYLEAKKNFLESLKPEDWEKNQYFLKLKEPKTKPKTLVQGWKNGFNLPRGIFTEPIRKWFMKHRENITVAELKRVGLVAKVIPLFFSEEYKDSVQPFYNYHFNVGNINKPDEKNFLNCEERRELLRKKKDEFKKMTDKEKEENPSYLEFKSWNKFERELRLVRNQDIVTWLLCMELFNKKKIKELNVEKIYLKNINTNTTKKEKNTEEKNGEEKNIKEKNNILNRIMPMRLPIKVYGRENFSKNKKKKIRRNTFFTVYIEEKGTKLLKQGNFKALERDRRLGGLFSFVKTPSKAESKSNTISKLRVEYELGEYQKARIEIIKDMLALEKTLIDKYNSLDTDNFNKMLTDWLELKGEPDKASFQNDVDLLIAVRNAFSHNQYPMRNRIAFANINPFSLSSANTSEEKGLGIANQLKDKTHKTIEKIIEIEKPIETKE

SEQ ID NO: 163 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 164 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFAVFFKPDDFVLAKNRKEQLISVADGKECLTVSGFAFFICLFLDREQASGMLSRIRGFKRTDENWARAVHETFCDLCIRHPHDRLESSNTKEALLLDMLNELNRCPRILYDMLPEEERAQFLPALDENSMNNLSENSLDEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMDQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELRLLDPSSGHPFLSATMETAHRYTEGFYKCYLEKKREWLAKIFYRPEQDENTKRRISVFFVPDGEARKLLPLLIRRRMKEQNDLQDWIRNKQAHPIDLPSHLFDSKVMELLKVKDGKKKWNEAFKDWWSTKYPDGMQPFYGLRRELNIHGKSVSYIPSDGKKFADCYTHLMEKTVRDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 165 - protein, 1115 aa, Bacteroides pyogenes
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETAECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTSGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPLPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 166 - protein, 668 aa, unidentified
  source 1..668: note = Description of Unknown: Cas13c sequence
MTEKKSIIFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKIAFGFLFCEFDGNISKFKDEFDASNQEKIIQYHKNGEKYLTYFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKIKKFL

SEQ ID NO: 167 - protein, 796 aa, unidentified
  source 1..796: note = Description of Unknown: Cas13c sequence
MEKDKKGEKIDISQEMIEEDLRKILILFSRLRHSMVHYDYEFYQALYSGKDFVISDKNNLENRMISQLLDLNIFKELSKVKLIKDKAISNYLDKNTTIHVLGQDIKAIRLLDIYRDICGSKNGFNKFINTMITISGEEDREYKEKVIEHFNKKMENLSTYLEKLEKQDNAKRNNKRVYNLLKQKLIEQQKLKEWFGGPYVYDIHSSKRYKELYIERKKLVDRHSKLFEEGLDEKNKKELTKINDELSKLNSEMKEMTKLNSKYRLQYKLQLAFGFILEEFDLNIDTFINNFDKDKDLIISNFMKKRDIYLNRVLDRGDNRLKNIIKEYKFRDTEDIFCNDRDNNLVKLYILMYILLPVEIRGDFLGFVKKNYYDMKHVDFIDKKDKEDKDTFFHDLRLFEKNIRKLEITDYSLSSGFLSKEHKVDIEKKINDFINRNGAMKLPEDITIEEFNKSLILPIMKNYQINFKLLNDIEISALFKIAKDRSITFKQAIDEIKNEDIKKNSKKNDKNNHKDKNINFTQLMKRALHEKIPYKAGMYQIRNNISHIDMEQLYIDPLNSYMNSNKNNITISEQIEKIIDVCVTGGVTGKELNNNIINDYYMKKEKLVFNLKLRKQNDIVSIESQEKNKREEFVFKKYGLDYKDGEINIIEVIQKVNSLQEELRNIKETSKEKLKNKETLFRDISLINGTIRKNINFKIKEMVLDIVRMDEIRHINIHIYYKGENYTRSNIIKFKYAIDGENKKYYLKQHEINDINLELKDKFVTLICNMDKHPNKNKQTINLESNYIQNVKFIIP

SEQ ID NO: 168 - protein, 897 aa, unidentified
  source 1..897: note = Description of Unknown: Cas13c sequence
MENKGNNKKIDFDENYNILVAQIKEYFTKEIENYNNRIDNIIDKKELLKYSEKKEESEKNKKLEELNKLKSQKLKILTDEEIKADVIKIIKIFSDLRHSLMHYEYKYFENLFENKKNEELAELLNLNLFKNLTLLRQMKIENKTNYLEGREEFNIIGKNIKAKEVLGHYNLLAEQKNGFNNFINSFFVQDGTENLEFKKLIDEHFVNAKKRLERNIKKSKKLEKELEKMEQHYQRLNCAYVWDIHTSTTYKKLYNKRKSLIEEYNKQINEIKDKEVITAINVELLRIKKEMEEITKSNSLFRLKYKMQIAYAFLEIEFGGNIAKFKDEFDCSKMEEVQKYLKKGVKYLKYYKDKEAQKNYEFPFEEIFENKDTHNEEWLENTSENNLFKFYILTYLLLPMEFKGDFLGVVKKHYYDIKNVDFTDESEKELSQVQLDKMIGDSFFHKIRLFEKNTKRYEIIKYSILTSDEIKRYFRLLELDVPYFEYEKGTDEIGIFNKNIILTIFKYYQIIFRLYNDLEIHGLFNISSDLDKILRDLKSYGNKNINFREFLYVIKQNNNSSTEEEYRKIWENLEAKYLRLHLLTPEKEEIKTKTKEELEKLNEISNLRNGICHLNYKEIIEEILKTEISEKNKEATLNEKIRKVINFIKENELDKVELGFNFINDFFMKKEQFMFGQIKQVKEGNSDSITTERERKEKNNKKLKETYELNCDNLSEFYETSNNLRERANSSSLLEDSAFLKKIGLYKVKNNKVNSKVKDEEKRIENIKRKLLKDSSDIMGMYKAEVVKKLKEKLILIFKHDEEKRIYVTVYDTSKAVPENISKEILVKRNNSKEEYFFEDNNKKYVTEYYTLEITETNELKVIPAKKLEGKEFKTEKNKENKLMLNNHYCFNVKIIY

SEQ ID NO: 169 - protein, 919 aa, unidentified
  source 1..919: note = Description of Unknown: Cas13c sequence
MEEIKHKKNKSSIIRVIVSNYDMTGIKEIKVLYQKQGGVDTFNLKTIINLESGNLEIISCKPKEREKYRYEFNCKTEINTISITKKDKVLKKEIRKYSLELYFKNEKKDTVVAKVTDLLKAPDKIEGERNHLRKLSSSTERKLLSKTLCKNYSEISKTPIEEIDSIKIYKIKRFLNYRSNFLIYFALINDFLCAGVKEDDINEVWLIQDKEHTAFLENRIEKITDYIFDKLSKDIENKKNQFEKRIKKYKTSLEELKTETLEKNKTFYIDSIKTKITNLENKITELSLYNSKESLKEDLIKIISIFTNLRHSLMHYDYKSFENLFENIENEELKNLLDLNLFKSIRMSDEFKTKNRTNYLDGTESFTIVKKHQNLKKLYTYYNNLCDKKNGFNTFINSFFVTDGIENTDFKNLIILHFEKEMEEYKKSIEYYKIKISNEKNKSKKEKLKEKIDLLQSELINMREHKNLLKQIYFFDIHNSIKYKELYSERKNLIEQYNLQINGVKDVTAINHINTKLLSLKNKMDKITKQNSLYRLKYKLKIAYSFLMIEFDGDVSKFKNNFDPTNLEKRVEYLDKKEEYLNYTAPKNKFNFAKLEEELQKIQSTSEMGADYLNVSPENNLFKFYILTYIMLPVEFKGDFLGFVKNHYYNIKNVDFMDESLLDENEVDSNKLNEKIENLKDSSFFNKIRLFEKNIKKYEIVKYSVSTQENMKEYFKQLNLDIPYLDYKSTDEIGIFNKNMILPIFKYYQNVFKLCNDIEIHALLALANKKQQNLEYAIYCCSKKNSLNYNELLKTFNRKTYQNLSFIRNKIAHLNYKELFSDLFNNELDLNTKVRCLIEFSQNNKFDQIDLGMNFINDYYMKKTRFIFNQRRLRDLNVPSKEKIIDGKRKQQNDSNNELLKKYGLSRTNIKDIFNKAWY

SEQ ID NO: 170 - protein, 1110 aa, unidentified
  source 1..1110: note = Description of Unknown: Cas13c sequence
MKVRYRKQAQLDTFIIKTEIVNNDIFIKSIIEKAREKYRYSFLFDGEEKYHFKNKSSVEIVKNDIFSQTPDNMIRNYKITLKISEKNPRVVEAEIEDLMNSTILKDGRRSARREKSMTERKLIEEKVAENYSLLANCPIEEVDSIKIYKIKRFLTYRSNMLLYFASINSFLCEGIKGKDNETEEIWHLKDNDVRKEKVKENFKNKLIQSTENYNSSLKNQIEEKEKLSSKEFKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTKLLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSEKPFEVFLEELKDKMIGKQLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKKSEKEEILRKRYHLINKKNEQIKDEHEAQSQLYEKILSLQKIFSCDKNNFYRRLKEEKLLFLEKQGKKKISMKEIKDKIASDISDLLGILKKEITRDIKDKLTEKFRYCEEKLLNISFYNHQDKKKEEGIRVFLIRDKNSDNFKFESILDDGSNKIFISKNGKEITIQCCDKVLETLMIEKNTLKISSNGKIISLIPHYSYSIDVKY

SEQ ID NO: 171 - RNA, 41 nt, synthetic construct
  source 1..41: mol_type = other RNA; note = Description of Artificial Sequence: Synthetic oligonucleotide
taatttctactaagtgtagatcccccagcgcttcagcgttc

SEQ ID NO: 172 - RNA, 41 nt, synthetic construct
  source 1..41: mol_type = other RNA; note = Description of Artificial Sequence: Synthetic oligonucleotide
taatttctactaagtgtagatttgctttcgtggtattcttg

SEQ ID NO: 173 - RNA, 41 nt, synthetic construct
  source 1..41: mol_type = other RNA; note = Description of Artificial Sequence: Synthetic oligonucleotide
taatttctactaagtgtagatgtggtattcttgctagttac

SEQ ID NO: 174 - RNA, 41 nt, synthetic construct
  source 1..41: mol_type = other RNA; note = Description of Artificial Sequence: Synthetic oligonucleotide
taatttctactaagtgtagatctccaagtgcctctgcattc

SEQ ID NO: 175 - RNA, 41 nt, synthetic construct
  source 1..41: mol_type = other RNA; note = Description of Artificial Sequence: Synthetic oligonucleotide
taatttctactaagtgtagatgcaatgttgttccttgagga

SEQ ID NO: 176 - RNA, 41 nt, synthetic construct
  source 1..41: mol_type = other RNA; note = Description of Artificial Sequence: Synthetic oligonucleotide
taatttctactaagtgtagatcacataccgcagacggtaca

SEQ ID NO: 177 - RNA, 41 nt, synthetic construct
  source 1..41: mol_type = other RNA; note = Description of Artificial Sequence: Synthetic oligonucleotide
taatttctactaagtgtagatgacctgcgagcgggttctga

SEQ ID NO: 178 - RNA, 41 nt, synthetic construct
  source 1..41: mol_type = other RNA; note = Description of Artificial Sequence: Synthetic oligonucleotide
taatttctactaagtgtagataattacttgggtgtgaccct

SEQ ID NO: 179 - DNA, 931 bp, Severe acute respiratory syndrome coronavirus 2
  source 1..931: mol_type = unassigned DNA
ccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtg

SEQ ID NO: 180 - DNA, 931 bp, unidentified
  source 1..931: mol_type = unassigned DNA; note = Description of Unknown: SARS-CoV N-gene sequence
ccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtg

SEQ ID NO: 181 - DNA, 931 bp, unidentified
  source 1..931: mol_type = unassigned DNA; note = Description of Unknown: Bat-SL-CoVZC45 N-gene sequence
ccaaattggctactaccgtagagctaccagacgaattcgtggtggtgacggtaaaatgaaagagctcagccccagatggtatttttactatctaggaactggaccagaagctggacttccctatggtgctaacaaagaaggcatcatatgggttgcaactgagggagccttaaacacaccgaaagaccacattggcacccgcaatcctgctaacaatgctgcaatcgtgctacaacttcctcaaggaacaacattgccaaaaggcttctacgcagaagggagcagaggcggcagtcaagcttcttcacgctcctcatcacgtagtcgcaacagttcaagaaactcaactccaggcagcagtaggggaacttctcctgctagaatggctggcaatggcggtgacactgctcttgctttgctgctgctagataggttgaaccagcttgagaacaaagtatctggcaaaggccaacaacaacagggccaaactgtcactaagaaatctgctgctgaggcatctaaaaagcctcgccaaaaacgtactgctacaaaacagtacaacgtcactcaagcatttgggagacgtggtccagaacaaacccaaggaaattttggggaccaagaattaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagataacgtcatactgctgaataagcacattgacgcatacaaaacattcccaccaacagagcctaaaaaggacaaaaagaaaaaggctgatgaacttcaggctttaccgcagagacagaagaaacaacaaactgtg

SEQ ID NO: 182 - DNA, 598 bp, Severe acute respiratory syndrome coronavirus 2
  source 1..598: mol_type = unassigned DNA
actattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatt

SEQ ID NO: 183 - DNA, 595 bp, unidentified
  source 1..595: mol_type = unassigned DNA; note = Description of Unknown: SARS-CoV E-gene sequence
tttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggatt

SEQ ID NO: 184 - DNA, 598 bp, unidentified
  source 1..598: mol_type = unassigned DNA; note = Description of Unknown: Bat-SL-CoVZC45 N-gene sequence
attactaccagctgtactcaacacaagtgagtacagacactggtgttgaacatgttactttcttcatctacaataaaattgttgatgagcctgaagaacatgttcaaattcacacaatcgacggtacatctggagttgttaatccagcaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgcttttgtggtattcttgctagtcacactagccatccttactgcgcttcgattgtgtgcgtactgctgcaatattgttaacgtgagtcttgtaaaaccttctttttacgtttactctcgtgttaaaaatctgaattcttctagagttcctgatcttttggtctaaacgaactaaatattatattagtctttctgtttggaactttaattttagccatgtcaggtgacaacggtaccattaccgttgaagagcttaaaaagctcttagaacaatggaacctagtaataggattcttgtttcttacatggatt

SEQ ID NO: 185 - DNA, 21 bp, synthetic construct
  source 1..21: mol_type = other DNA; note = Description of Artificial Sequence: Synthetic oligonucleotide
taattaattaattaattaatt

SEQ ID NO: 186 - DNA, 52 bp, synthetic construct
  source 1..52: mol_type = other DNA; note = Description of Artificial Sequence: Synthetic primer
aattctaatacgactcactatagggccaaattggctactaccgaagagctac

SEQ ID NO: 187 - DNA, 30 bp, synthetic construct
  source 1..30: mol_type = other DNA; note = Description of Artificial Sequence: Synthetic primer
cacagtttgctgtttcttctgtctctgcgg

SEQ ID NO: 188 - DNA, 54 bp, synthetic construct
  source 1..54: mol_type = other DNA; note = Description of Artificial Sequence: Synthetic primer
aattctaatacgactcactatagggctggtgttgaacatgttaccttcttcatc

SEQ ID NO: 189 - DNA, 23 bp, synthetic construct
  source 1..23: mol_type = other DNA; note = Description of Artificial Sequence: Synthetic primer
cctattactaggttccattgttc

SEQ ID NO: 190 - DNA, 26 bp, synthetic construct
  source 1..26: mol_type = other DNA; note = Description of Artificial Sequence: Synthetic primer
acaggtacgttaatagttaatagcgt

SEQ ID NO: 191 - DNA, 22 bp, synthetic construct
  source 1..22: mol_type = other DNA; note = Description of Artificial Sequence: Synthetic primer
atattgcagcagtacgcacaca

SEQ ID NO: 192 - DNA, 20 bp, synthetic construct
  source 1..20: mol_type = other DNA; note = Description of Artificial Sequence: Synthetic primer
ttacaaacattggccgcaaa

SEQ ID NO: 193 - DNA, 18 bp, synthetic construct
  source 1..18: mol_type = other DNA; note = Description of Artificial Sequence: Synthetic primer
gcgcgacattccgaagaa

SEQ ID NO: 194 - DNA, 18 bp, synthetic construct
  source 1..18: mol_type = other DNA; note = Description of Artificial Sequence: Synthetic primer
aacacaagctttcggcag

SEQ ID NO: 195 - DNA, 22 bp, synthetic construct
  source 1..22: mol_type = other DNA; note = Description of Artificial Sequence: Synthetic primer
gaaatttggatctttgtcatcc

SEQ ID NO: 196 - DNA, 41 bp, synthetic construct
  source 1..41: mol_type = other DNA; note = Description of Artificial Sequence: Synthetic primer
tgcggccaatgtttgtaatcagccaaggaaattttggggac

SEQ ID NO: 197 - DNA, 39 bp, synthetic construct
  source 1..39: mol_type = other DNA; note = Description of Artificial Sequence: Synthetic primer
cgcattggcatggaagtcactttgatggcacctgtgtag

SEQ ID NO: 198 - DNA, 19 bp, synthetic construct
  source 1..19: mol_type = other DNA; note = Description of Artificial Sequence: Synthetic primer
ttccttgtctgattagttc

SEQ ID NO: 199 - DNA, 18 bp, synthetic construct
  source 1..18: mol_type = other DNA; note = Description of Artificial Sequence: Synthetic primer
accttcgggaacgtggtt

SEQ ID NO: 200 - DNA, 18 bp, synthetic construct
  source 1..18: mol_type = other DNA; note = Description of Artificial Sequence: Synthetic primer
ccgacgacgactactagc

SEQ ID NO: 201 - DNA, 23 bp, synthetic construct
  source 1..23: mol_type = other DNA; note = Description of Artificial Sequence: Synthetic primer
agagtaaacgtaaaaagaaggtt

SEQ ID NO: 202 - DNA, 41 bp, synthetic construct
  source 1..41: mol_type = other DNA; note = Description of Artificial Sequence: Synthetic primer
acctgtctcttccgaaacgaatttgtaagcacaagctgatg

SEQ ID NO: 203 - DNA, 41 bp, synthetic construct
  source 1..41: mol_type = other DNA; note = Description of Artificial Sequence: Synthetic primer
ctagccatccttactgcgctactcacgttaacaatattgca

SEQ ID NO: 204 - DNA, 19 bp, synthetic construct
  source 1..19: mol_type = other DNA; note = Description of Artificial Sequence: Synthetic primer
tcgattgtgtgcgtactgc

SEQ ID NO: 205 - DNA, 19 bp, synthetic construct
  source 1..19: mol_type = other DNA; note = Description of Artificial Sequence: Synthetic primer
tgagtacataagttcgtac

SEQ ID NO: 206 - DNA, 17 bp, synthetic construct
  source 1..17: mol_type = other DNA; note = Description of Artificial Sequence: Synthetic primer
ttgatgagctggagcca

SEQ ID NO: 207 - DNA, 18 bp, synthetic construct
  source 1..18: mol_type = other DNA; note = Description of Artificial Sequence: Synthetic primer
caccctcaatgcagagtc

SEQ ID NO: 208 - DNA, 41 bp, synthetic construct
  source 1..41: mol_type = other DNA; note = Description of Artificial Sequence: Synthetic primer
gtgtgaccctgaagactcggttttagccactgactcggatc

SEQ ID NO: 209 - DNA, 45 bp, synthetic construct
  source 1..45: mol_type = other DNA; note = Description of Artificial Sequence: Synthetic primer
cctccgtgatatggctcttcgtttttttcttacatggctctggtc

SEQ ID NO: 210 - DNA, 20 bp, synthetic construct
  source 1..20: mol_type = other DNA; note = Description of Artificial Sequence: Synthetic primer
atgtggatggctgagttgtt

SEQ ID NO: 211 - DNA, 20 bp, synthetic construct
  source 1..20: mol_type = other DNA; note = Description of Artificial Sequence: Synthetic primer
catgctgagtactggacctc

SEQ ID NO: 212 - DNA, 18 bp, synthetic construct
  source 1..18: mol_type = other DNA; note = Description of Artificial Sequence: Synthetic primer
cacatccgagtcttcagg

SEQ ID NO: 213 - DNA, 19 bp, synthetic construct
  source 1..19: mol_type = other DNA; note = Description of Artificial Sequence: Synthetic primer
ggcaatagttacagaccgc

SEQ ID NO: 214 - DNA, 44 bp, synthetic construct
  source 1..44: mol_type = other DNA; note = Description of Artificial Sequence: Synthetic primer
tccagtactcagcatgcgaagcacccaagtaattgaaaagacac

SEQ ID NO: 215 - DNA, 39 bp, synthetic construct
  source 1..39: mol_type = other DNA; note = Description of Artificial Sequence: Synthetic primer
ctggaagcccaaaggactctatacacacactcaggaagg

SEQ ID NO: 216 - DNA, 20 bp, synthetic construct
  source 1..20: mol_type = other DNA; note = Description of Artificial Sequence: Synthetic primer
cggaggggataagtggagga

SEQ ID NO: 217 - DNA, 17 bp, synthetic construct
  source 1..17: mol_type = other DNA; note = Description of Artificial Sequence: Synthetic primer
gcattgagggtgggggt

SEQ ID NO: 218 - RNA, 41 nt, synthetic construct
  source 1..41: mol_type = other RNA; note = Description of Artificial Sequence: Synthetic oligonucleotide
taatttctactaagtgtagatttacatggctctggtccgag

SEQ ID NO: 219 - DNA, 41 bp, synthetic construct
  source 1..41: mol_type = other DNA; note = Description of Artificial Sequence: Synthetic oligonucleotide; note = Description of Combined DNA/RNA Molecule: Synthetic oligonucleotide
  modified_base 25: mod_base = OTHER; note = thymine
  modified_base 26: mod_base = OTHER; note = thymine
  modified_base 41: mod_base = OTHER; note = thymine
taatttctactaagtgtagatggcttccagggaacaggcct

SEQ ID NO: 220 - DNA, 2026 bp, Homo sapiens
  source 1..2026: mol_type = unassigned DNA
ggagtattgaatagttgggaattggaacccctccagggggaaccaaacattgtcgttcagaagaagacaaagagagattgaaatgaagctgttgatttcaacacacaaattctggtggtagatgaaagcaaagcaagtaagtttctccgaatccctagtcaactggaggtagagacggactgcgcaggttaactacagctcccagcatgcctgaggggcgggctcagcggctgcgcagactggcgcgcgcggacggtcatgggacttcagcatggcggtgtttgcagatttggacctgcgagcgggttctgacctgaaggctctgcgcggacttgtggagacagccgctcaccttggctattcagttgttgctatcaatcatatcgttgactttaaggaaaagaaacaggaaattgaaaaaccagtagctgtttctgaactcttcacaactttgccaattgtacagggaaaatcaagaccaattaaaattttaactagattaacaattattgtctcggatccatctcactgcaatgttttgagagcaacttcttcaagggcccggctctatgatgttgttgcagtttttccaaagacagaaaagctttttcatattgcttgcacacatttagatgtggatttagtctgcataactgtaacagagaaactaccattttacttcaaaagacctcctattaatgtggcgattgaccgaggcctggcttttgaacttgtctatagccctgctatcaaagactccacaatgagaaggtatacaatttccagtgccctcaatttgatgcaaatctgcaaaggaaagaatgtaattatatctagtgctgcagaaaggcctttagaaataagagggccatatgacgtggcaaatctaggcttgctgtttgggctctctgaaagtgacgccaaggctgcggtgtccaccaactgccgagcagcgcttctccatggagaaactagaaaaactgcttttggaattatctctacagtgaagaaacctcggccatcagaaggagatgaagattgtcttccagcttccaagaaagccaagtgtgagggctgaaaagaatgccccagtctctgtcagcactcccttcttcccttttatagttcatcagccacaacaaaaataaaacctttgtgtgatttactgttttcatttggagctagaaatcaatagtctataaaaacagttttacttgcaatccattaaaacaacaaacgaaacctagtgaagcatctttttaaaaggctgccagcttaatgaatttagatgtactttaagagagaaagactggttatttctcctttgtgtaagtgataaacaacagcaaatatacttgaataaaatgtttcaggtatttttgtttcattttgtttttgagatagggtctttgttgctcaggctggagtacagtggcataatcacagctcactgcaacctcaatcctgggctcaagtgatcctcccgcttcagcctctcaagcagcgggaactacaggtgtgcactaccacacctggctattttttttttttttttttttttcccttgtagagacatggtctcactatgttgctgaggctggtctcaaactcctaggatcaagccatcctcccgctttggcctcctaaagtgctgggattacatgagccaccacatgcagccagatgtttgaatattttaagagcttctttcgaaagtttcttgttcatactcaaatagtagttattttgaagatattcaaacttatattgaagaagtgactttagttcctcttgttttaagcttctttcatgtattcaaatcagcatttttttctaagaaattgctatagaatttgtggaaggagagaggatacacatgtaaaattacatctggtctcttccttcactgcttcatgcctacgtaaggtctttgaaataggattccttacttttagttagaaacccctaaaacgctaatattgattttcctgatagctgtattaaaaatagcaaagcatcggactga

SEQ ID NO: 221 - protein, 707 aa, synthetic construct
  source 1..707: note = Description of Artificial Sequence: Synthetic polypeptide
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 222 - protein, 757 aa, synthetic construct
  source 1..757: note = Description of Artificial Sequence: Synthetic polypeptide
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 223 - protein, 765 aa, synthetic construct
  source 1..765: note = Description of Artificial Sequence: Synthetic polypeptide
MYILEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 224 - protein, 766 aa, synthetic construct
  source 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 225 - protein, 812 aa, synthetic construct
  source 1..812: note = Description of Artificial Sequence: Synthetic polypeptide
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 226 - protein, 812 aa, synthetic construct
  source 1..812: note = Description of Artificial Sequence: Synthetic polypeptide
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHKGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 227 - protein, 793 aa, synthetic construct
  source 1..793: note = Description of Artificial Sequence: Synthetic polypeptide
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 228 - protein, 441 aa, synthetic construct
  source 1..441: note = Description of Artificial Sequence: Synthetic polypeptide
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 229 - protein, 812 aa, synthetic construct
  source 1..812: note = Description of Artificial Sequence: Synthetic polypeptide
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 230 - protein, 812 aa, synthetic construct
  source 1..812: note = Description of Artificial Sequence: Synthetic polypeptide
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 231 - protein, 761 aa, synthetic construct
  source 1..761: note = Description of Artificial Sequence: Synthetic polypeptide
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 232 - protein, 717 aa, synthetic construct
  source 1..717: note = Description of Artificial Sequence: Synthetic polypeptide
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 233 - protein, 781 aa, synthetic construct
  source 1..781: note = Description of Artificial Sequence: Synthetic polypeptide
MRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 234 - protein, 793 aa, synthetic construct; identical to SEQ ID NO: 227
  source 1..793: note = Description of Artificial Sequence: Synthetic polypeptide

SEQ ID NO: 235 - protein, 717 aa, synthetic construct; identical to SEQ ID NO: 232
  source 1..717: note = Description of Artificial Sequence: Synthetic polypeptide

SEQ ID NO: 236 - protein, 761 aa, synthetic construct; identical to SEQ ID NO: 231
  source 1..761: note = Description of Artificial Sequence: Synthetic polypeptide

SEQ ID NO: 237 - protein, 765 aa, synthetic construct
  source 1..765: note = Description of Artificial Sequence: Synthetic polypeptide
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 238 - protein, 766 aa, synthetic construct
  source 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 239 - protein, 870 aa, synthetic construct
  source 1..870: note = Description of Artificial Sequence: Synthetic polypeptide
MLVRTSTLVQDNKNSRSASRAFLKKPKMPKNKHIKEPTELAKLIRELFPGQRFTRAINTQAGKILKHKGRDEVVEFLKNKGIDKEQFMDFRPPTKARIVATSGAIEEFSYLRVSMAIQECCFGKYKFPKEKVNGKLVLETVGLTKEELDDFLPKKYYENKKSRDRFFLKTGICDYGYTYAQGLNEIFRNTRAIYEGVFTKVNNRNEKRREKKDKYNEERRSKGLSEEPYDEDESATDESGHLINPPGVNLNIWTCEGFCKGPYVTKLSGTPGYEVILPKVFDGYNRDPNEIISCGITDRFAIPEGEPGHIPWHQRLEIPEGQPGYVPGHQRFADTGQNNSGKANPNKKGRMRKYYGHGTKYTQPGEYQEVFRKGHREGNKRRYWEEDFRSEAHDCILYVIHIGDDWVVCDLRGPLRDAYRRGLVPKEGITTQELCNLFSGDPVIDPKHGVVTFCYKNGLVRAQKTISAGKKSRELLGALTSQGPIALIGVDLGQTEPVGARAFIVNQARGSLSLPTLKGSFLLTAENSSSWNVFKGEIKAYREAIDDLAIRLKKEAVATLSVEQQTEIESYEAFSAEDAKQLACEKFGVDSSFILWEDMTPYHTGPATYYFAKQFLKKNGGNKSLIEYIPYQKKKSKKTPKAVLRSDYNIACCVRPKLLPETRKALNEAIRIVQKNSDEYQRLSKRKLEFCRRVVNYLVRKAKKLTGLERVIIAIEDLKSLEKFFTGSGKRDNGWSNFFRPKKENRWFIPAFHKAFSELAPNRGFYVIECNPARTSITDPDCGYCDGDNRDGIKFECKKCGAKHHTDLDVAPLNIAIVAVTGRPMPKTVSNKSKRERSGGEKSVGASRKRNHRKSKANQEMLDATSSAAE

SEQ ID NO: 240 - protein, 830 aa, synthetic construct
  source 1..830: note = Description of Artificial Sequence: Synthetic polypeptide
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 241 - protein, 790 aa, synthetic construct
  source 1..790: note = Description of Artificial Sequence: Synthetic polypeptide
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLNADLDVATTNLVRVALTGKVMPRSERSGDAQTPGPARKARTGKIKGSKPTSAPQGATQTDAKAHLSQTGV

SEQ ID NO: 242 - protein, 790 aa, synthetic construct
  source 1..790: note = Description of Artificial Sequence: Synthetic polypeptide
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLHADLDVATTNLVRVALTGKVMPRSERSGDAQTPGPARKARTGKIKGSKPTSAPQGATQTDAKAHLSQTGV

SEQ ID NO: 243 - protein, 782 aa, synthetic construct
  source 1..782: note = Description of Artificial Sequence: Synthetic polypeptide
MKTEKPKTALTLLREEVFPGKKYRLDVLKEAGKKLSTKGREATIEFLTGKDEERPQNFQPPAKTSIVAQSRPFDQWPIVQVSLAVQKYIYGLTQSEFEANKKALYGETGKAISTESRRAWFEATGVDNFGFTAAQGINPIFSQAVARYEGVIKKVENRNEKKLKKLTKKNLLRLESGEEIEDFEPEATFNEEGRLLQPPGANPNIYCYQQISPRIYDPSDPKGVILPQIYAGYDRKPEDIISAGVPNRLAIPEGQPGYIPEHQRAGLKTQGRIRCRASVEAKARAAILAVVHLGEDWVVLDLRGLLRNVYWRKLASPGTLTLKGLLDFFTGGPVLDARRGIATFSYTLKSAAAVHAENTYKGKGTREVLLKLTENNSVALVTVDLGQRNPLAAMIARVSRTSQGDLTYPESVEPLTRLFLPDPFLEEVRKYRSSYDALRLSIREAAIASLTPEQQAEIRYIEKFSAGDAKKNVAEVFGIDPTQLPWDAMTPRTTYISDLFLRMGGDRSRVFFEVPPKKAKKAPKKPPKKPAGPRIVKRTDGMIARLREIRPRLSAETNKAFQEARWEGERSNVAFQKLSVRRKQFARTVVNHLVQTAQKMSRCDTVVLGIEDLNVPFFHGRGKYQPGWEGFFRQKKENRWLINDMHKALSERGPHRGGYVLELTPFWTSLRCPKCGHTDSANRDGDDFVCVKCGAKLHSDLEVATANLALVAITGQSIPRPPREQSSGKKSTGTARMKKTSGETQGKGSKACVSEALNKIEQGTARDPVYNPLNSQVSCPAP

SEQ ID NO: 244 - protein, 774 aa, synthetic construct
  source 1..774: note = Description of Artificial Sequence: Synthetic polypeptide
VYNPDMKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 245 - protein, 769 aa, synthetic construct
  source 1..769: note = Description of Artificial Sequence: Synthetic polypeptide
MKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 246 - protein, 767 aa, synthetic construct
  source 1..767: note = Description of Artificial Sequence: Synthetic polypeptide
VIKTHFPAGRFRKDHQKTAGKKLKHEGEEACVEYLRNKVSDYPPNFKPPAKGTIVAQSRPFSEWPIVRASEAIQKYVYGLTVAELDVFSPGTSKPSHAEWFAKTGVENYGYRQVQGLNTIFQNTVNRFKGVLKKVENRNKKSLKRQEGANRRRVEEGLPEVPVTVESATDDEGRLLQPPGVNPSIYGYQGVAPRVCTDLQGFSGMSVDFAGYRRDPDAVLVESLPEGRLSIPKGERGYVPEWQRDPERNKFPLREGSRRQRKWYSNACHKPKPGRTSKYDPEALKKASAKDALLVSISIGEDWAIIDVRGLLRDARRRGFTPEEGLSLNSLLGLFTEYPVFDVQRGLITFTYKLGQVDVHSRKTVPTFRSRALLESLVAKEEIALVSVDLGQTNPASMKVSRVRAQEGALVAEPVHRMFLSDVLLGELSSYRKRMDAFEDAIRAQAFETMTPEQQAEITRVCDVSVEVARRRVCEKYSISPQDVPWGEMTGHSTFIVDAVLRKGGDESLVYFKNKEGETLKFRDLRISRMEGVRPRLTKDTRDALNKAVLDLKRAHPTFAKLAKQKLELARRCVNFIEREAKRYTQCERVVFVIEDLNVGFFHGKGKRDRGWDAFFTAKKENRWVIQALHKAFSDLGLHRGSYVIEVTPQRTSMTCPRCGHCDKGNRNGEKFVCLQCGATLHADLEVATDNIERVALTGKAMPKPPVRERSGDVQKAGTARKARKPLKPKQKTEPSVQEGSSDDGVDKSPGDASRNPVYNPSDTLSI

SEQ ID NO: 247 - protein, 763 aa, synthetic construct
  source 1..763: note = Description of Artificial Sequence: Synthetic polypeptide
MAKAKTLAALLRELLPGQHLAPHHRWVANKLLMTSGDAAAFVIGKSVSDPVRGSFRKDVITKAGRIFKKDGPDAAAAFLDGKWEDRPPNFQPPAKAAIVAISRSFDEWPIVKVSCAIQQYLYALPVQEFESSVPEARAQAHAAWFQDTGVDDCNFKSTQGLNAIFNHGKRTYEGVLKKAQNRNDKKNLRLERINAKRAEAGQAPLVAGPDESPTDDAGCLLHPPGINANIYCYQQVSPRPYEQSCGIQLPPEYAGYNRLSNVAIPPMPNRLDIPQGQPGYVPEHHRHGIKKFGRVRKRYGVVPGRNRDADGKRTRQVLTEAGAAAKARDSVLAVIRIGDDWTVVDLRGLLRNAQWRKLVPDGGITVQGLLDLFTGDPVIDPRRGVVTFIYKADSVGIHSEKVCRGKQSKNLLERLCAMPEKSSTRLDCARQAVALVSVDLGQRNPVAARFSRVSLAEGQLQAQLVSAQFLDDAMVAMIRSYREEYDRFESLVREQAKAALSPEQLSEIVRHEADSAESVKSCVCAKFGIDPAGLSWDKMTSGTWRIADHVQAAGGDVEWFFFKTCGKGKEIKTVRRSDFNVAKQFRLRLSPETRKDWNDAIWELKRGNPAYVSFSKRKSEFARRVVNDLVHRARRAVRCDEVVFAIEDLNISFFHGKGQRQMGWDAFFEVKQENRWFIQALHKAFVERATHKGGYVLEVAPARTSTTCPECRHCDPESRRGEQFCCIKCRHTCHADLEVATFNIEQVALTGVSLPKRLSSTLL

SEQ ID NO: 248 - protein, 761 aa, synthetic construct
  source 1..761: note = Description of Artificial Sequence: Synthetic polypeptide
MSKEKTPPSAYAILKAKHFPDLDFEKKHKMMAGRMFKNGASEQEVVQYLQGKGSESLMDVKPPAKSPILAQSRPFDEWEMVRTSRLIQETIFGIPKRGSIPKRDGLSETQFNELVASLEVGGKPMLNKQTRAIFYGLLGIKPPTFHAMAQNILIDLAINIRKGVLKKVDNLNEKNRKKVKRIRDAGEQDVMVPAEVTAHDDRGYLNHPPGVNPTIPGYQGVVIPFPEGFEGLPSGMTPVDWSHVLVDYLPHDRLSIPKGSPGYIPEWQRPLLNRHKGRRHRSWYANSLNKPRKSRTEEAKDRQNAGKRTALIEAERLKGVLPVLMRFKEDWLIIDARGLLRNARYRGVLPEGSTLGNLIDLFSDSPRVDTRRGICTFLYRKGRAYSTKPVKRKESKETLLKLTEKSTIALVSIDLGQTNPLTAKLSKVRQVDGCLVAEPVLRKLIDNASEDGKEIARYRVAHDLLRARILEDAIDLLGIYKDEVVRARSDTPDLCKERVCRFLGLDSQAIDWDRMTPYTDFIAQAFVAKGGDPKVVTIKPNGKPKMFRKDRSIKNMKGIRLDISKEASSAYREAQWAIQRESPDFQRLAVWQSQLTKRIVNQLVAWAKKCTQCDTVVLAFEDLNIGMMHGSGKWANGGWNALFLHKQENRWFMQAFHKALTELSAHKGIPTIEVLPHRTSITCTQCGHCHPGNRDGERFKCLKCEFLANTDLEIATDNIERVALTGLPMPKGERSSAKRKPGGTRKTKKSKHSGNSPLAAE

SEQ ID NO: 249 - protein, 746 aa, synthetic construct
  source 1..746: note = Description of Artificial Sequence: Synthetic polypeptide
MEKAGPTSPLSVLIHKNFEGCRFQIDHLKIAGRKLAREGEAAAIEYLLDKKCEGLPPNFQPPAKGNVIAQSRPFTEWAPYRASVAIQKYIYSLSVDERKVCDPGSSSDSHEKWFKQTGVQNYGYTHVQGLNLIFKHALARYDGVLKKVDNRNEKNRKKAERVNSFRREEGLPEEVFEEEKATDETGHLLQPPGVNHSIYCYQSVRPKPFNPRKPGGISLPEAYSGYSLKPQDELPIGSLDRLSIPPGQPGYVPEWQRSQLTTQKHRRKRSWYSAQKWKPRTGRTSTFDPDRLNCARAQGAILAVVRIHEDWVVFDVRGLLRNALWRELAGKGLTVRDLLDFFTGDPVVDTKRGVVTFTYKLGKVDVHSLRTVRGKRSKKVLEDLTLSSDVGLVTIDLGQTNVLAADYSKVTRSENGELLAVPLSKSFLPKHLLHEVTAYRTSYDQMEEGFRRKALLTLTEDQQVEVTLVRDFSVESSKTKLLQLGVDVTSLPWEKMSSNTTYISDQLLQQGADPASLFFDGERDGKPCRHKKKDRTWAYLVRPKVSPETRKALNEALWALKNTSPEFESLSKRKIQFSRRCMNYLLNEAKRISGCGQVVFVIEDLNVRVHHGRGKRAIGWDNFFKPKRENRWFMQALHKAASELAIHRGMHIIEACPARSSITCPKCGHCDPENRCSSDREKFLCVKCGAAFHADLEVATFNLRKVALTGTALPKSIDHSRDGLIPKGARNRKLKEPQANDEKACA

SEQ ID NO: 250 - protein, 735 aa, synthetic construct
  source 1..735: note = Description of Artificial Sequence: Synthetic polypeptide
MKEQSPLSSVLKSNFPGKKFLSADIRVAGRKLAQLGEAAAVEYLSPRQRDSVPNFRPPAFCTVVAKSRPFEEWPIYKASVLLQEQIYGMTGQEFEERCGSIPTSLSGLRQWASSVGLGAAMEGLHVQGMNLMVKNAINRYKGVLVKVENRNKKLVEANEAKNSSREERGLPPLRPPELGSAFGPDGRLVNPPGIDKSIRLYQGVSPVPVVKTTGRPTVHRLDIPAGEKGHVPLWQREAGLVKEGPRRRRMWYSNSNLKRSRKDRSAEASEARKADSVVVRVSVKEDWVDIDVRGLLRNVAWRGIERAGESTEDLLSLFSGDPVVDPSRDSVVFLYKEGVVDVLSKKVVGAGKSRKQLEKMVSEGPVALVSCDLGQTNYVAARVSVLDESLSPVRSFRVDPREFPSADGSQGVVGSLDRIRADSDRLEAKLLSEAEASLPEPVRAEIEFLRSERPSAVAGRLCLKLGIDPRSIPWEKMGSTTSFISEALSAKGSPLALHDGAPIKDSRFAHAARGRLSPESRKALNEALWERKSSSREYGVISRRKSEASRRMANAVLSESRRLTGLAVVAVNLEDLNMVSKFFHGRGKRAPGWAGFFTPKMENRWFIRSIHKAMCDLSKHRGITVIESRPERTSISCPECGHCDPENRSGERFSCKSCGVSLHADFEVATRNLERVALTGKPMPRRENLHSPEGATASRKTRKKPREATASTFLDLRSVLSSAENEGSGPAARAG

SEQ ID NO: 251 - protein, 725 aa, synthetic construct
  source 1..725: note = Description of Artificial Sequence: Synthetic polypeptide
MLPPSNKIGKSMSLKEFINKRNFKSSIIKQAGKILKKEGEEAVKKYLDDNYVEGYKKRDFPITAKCNIVASNRKIEDFDISKFSSFIQNYVFNLNKDNFEEFSKIKYNRKSFDELYKKIANEIGLEKPNYENIQGEIAVIRNAINIYNGVLKKVENRNKKIQEKNQSKDPPKLLSAFDDNGFLAERPGINETIYGYQSVRLRHLDVEKDKDIIVQLPDIYQKYNKKSTDKISVKKRLNKYNVDEYGKLISKRRKERINKDDAILCVSNFGDDWIIFDARGLLRQTYRYKLKKKGLCIKDLLNLFTGDPIINPTKTDLKEALSLSFKDGIINNRTLKVKNYKKCPELISELIRDKGKVAMISIDLGQTNPISYRLSKFTANNVAYIENGVISEDDIVKMKKWREKSDKLENLIKEEAIASLSDDEQREVRLYENDIADNTKKKILEKFNIREEDLDFSKMSNNTYFIRDCLKNKNIDESEFTFEKNGKKLDPTDACFAREYKNKLSELTRKKINEKIWEIKKNSKEYHKISIYKKETIRYIVNKLIKQSKEKSECDDIIVNIEKLQIGGNFFGGRGKRDPGWNNFFLPKEENRWFINACHKAFSELAPHKGIIVIESDPAYTSQTCPKCENCDKENRNGEKFKCKKCNYEANADIDVATENLEKIAKNGRRLIKNFDQLGERLPGAEMPGGARKRKPSKSLPKNGRGAGVGSEPELINQSPSQVIA

SEQ ID NO: 252 - protein, 718 aa, synthetic construct
  source 1..718: note = Description of Artificial Sequence: Synthetic polypeptide
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 253 - protein, 708 aa, synthetic construct
  source 1..708: note = Description of Artificial Sequence: Synthetic polypeptide
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 254 - protein, 491 aa, synthetic construct
  source 1..491: note = Description of Artificial Sequence: Synthetic polypeptide
VLLSDRIQYTDPSAPIPAMTVVDRRKIKKGEPGYVPPFMRKNLSTNKHRRMRLSRGQKEACALPVGLRLPDGKDGWDFIIFDGRALLRACRRLRLEVTSMDDVLDKFTGDPRIQLSPAGETIVTCMLKPQHTGVIQQKLITGKMKDRLVQLTAEAPIAMLTVDLGEHNLVACGAYTVGQRRGKLQSERLEAFLLPEKVLADFEGYRRDSDEHSETLRHEALKALSKRQQREVLDMLRTGADQARESLCYKYGLDLQALPWDKMSSNSTFIAQHLMSLGFGESATHVRYRPKRKASERTILKYDSRFAAEEKIKLTDETRRAWNEAIWECQRASQEFRCLSVRKLQLARAAVNWTLTQAKQRSRCPRVVVVVEDLNVRFMHGGGKRQEGWAGFFKARSEKRWFIQALHKAYTELPTNRGIHVMEVNPARTSITCTKCGYCDPENRYGEDFHCRNPKCKVRGGHVANADLDIATENLARVALSGPMPKAPKLK

SEQ ID NO: 255 - protein, 848 aa, synthetic construct
  source 1..848: note = Description of Artificial Sequence: Synthetic polypeptide
MTPSFGYQMIIVTPIHHASGAWATLRLLFLNPKTSGVMLGMTKTKSAFALMREEVFPGLLFKSADLKMAGRKFAKEGREAAIEYLRGKDEERPANFKPPAKGDIIAQSRPFDQWPIVQVSQAIQKYIFGLTKAEFDATKTLLYGEGNHPTTESRRRWFEATGVPDFGFTSAQGLNAIFSSALARYEGVIQKVENRNEKRLKKLSEKNQRLVEEGHAVEAYVPETAFHTLESLKALSEKSLVPLDDLMDKIDRLAQPPGINPCLYGYQQVAPYIYDPENPRGVVLPDLYLGYCRKPDDPITACPNRLDIPKGQPGYIPEHQRGQLKKHGRVRRFRYTNPQAKARAKAQTAILAVLRIDEDWVVMDLRGLLRNVYFREVAAPGELTARTLLDTFTGCPVLNLRSNVVTFCYDIESKGALHAEYVRKGWATRNKLLDLTKDGQSVALLSVDLGQRHPVAVMISRLKRDDKGDLSEKSIQVVSRTFADQYVDKLKRYRVQYDALRKEIYDAALVSLPPEQQAEIRAYEAFAPGDAKANVLSVMFQGEVSPDELPWDKMNTNTHYISDLYLRRGGDPSRVFFVPQPSTPKKNAKKPPAPRKPVKRTDENVSHMPEFRPHLSNETREAFQKAKWTMERGNVRYAQLSRFLNQIVREANNWLVSEAKKLTQCQTVVWAIEDLHVPFFHGKGKYHETWDGFFRQKKEDRWFVNVFHKAISERAPNKGEYVMEVAPYRTSQRCPVCGFVDADNRHGDHFKCLRCGVELHADLEVATWNIALVAVQGHGIAGPPREQSCGGETAGTARKGKNIKKNKGLADAVTVEAQDSEGGSKKDAGTARNPVYIPSESQVNCPAP

SEQ ID NO: 256 - protein, 781 aa, synthetic construct
  source 1..781: note = Description of Artificial Sequence: Synthetic polypeptide
MKPKTPKPPKTPVAALIDKHFPGKRFRASYLKSVGKKLKNQGEDVAVRFLTGKDEERPPNFQPPAKSNIVAQSRPIEEWPIHKVSVAVQEYVYGLTVAEKEACSDAGESSSSHAAWFAKTGVENFGYTSVQGLNKIFPPTFNRFDGVIKKVENRNEKKRQKATRINEAKRNKGQSEDPPEAEVKATDDAGYLLQPPGINHSVYGYQSITLCPYTAEKFPTIKLPEEYAGYHSNPDAPIPAGVPDRLAIPEGQPGHVPEEHRAGLSTKKHRRVRQWYAMANWKPKPKRTSKPDYDRLAKARAQGALLIVIRIDEDWVVVDARGLLRNVRWRSLGKREITPNELLDLFTGDPVLDLKRGVVTFTYAEGVVNVCSRSTTKGKQTKVLLDAMTAPRDGKKRQIGMVAVDLGQTNPIAAEYSRVGKNAAGTLEATPLSRSTLPDELLREIALYRKAHDRLEAQLREEAVLKLTAEQQAENARYVETSEEGAKLALANLGVDTSTLPWDAMTGWSTCISDHLINHGGDTSAVFFQTIRKGTKKLETIKRKDSSWADIVRPRLTKETREALNDFLWELKRSHEGYEKLSKRLEELARRAVNHVVQEVKWLTQCQDIVIVIEDLNVRNFHGGGKRGGGWSNFFTVKKENRWFMQALHKAFSDLAAHRGIPVLEVYPARTSITCLGCGHCDPENRDGEAFVCQQCGATFHADLEVATRNIARVALTGEAMPKAPAREQPGGAKKRGTSRRRKLTEVAVKSAEPTIHQAKNQQLNGTSRDPVYKGSELPAL

SEQ ID NO: 257 - protein, 767 aa, synthetic construct
  source 1..767: note = Description of Artificial Sequence: Synthetic polypeptide
MSEITDLLKANFKGKTFKSADMRMAGRILKKSGAQAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRGVQTTGTARNPGRSLKSNKNPSEPKRVLQSKTRKKITSTETQNEPLATDLKT

SEQ ID NO: 258 - protein, 760 aa, synthetic construct
  source 1..760: note = Description of Artificial Sequence: Synthetic polypeptide
MTPKTESPLSALCKKHFPGKRFRTNYLKDAGKILKKHGEDAVVAFLSDKQEDEPANFCPPAKVHILAQSRPFEDWPINLASKAIQTYVYGLTADERKTCEPGTSKESHDRWFKETGVDHHGFTSVQGLNLIFKHTLNRYDGVIKKVETRNEKRRSSVVRINEKKAAEGLPLIAAEAEETAFGEDGRLLQPPGVNHSIYCFQQVSPQPYSSKKHPQVVLPHAVQGVDPDAPIPVGRPNRLDIPKGQPGYVPEWQRPHLSMKCKRVRMWYARANWRRKPGRRSVLNEARLKEASAKGALPIVLVIGDDWLVMDARGLLRSVFWRRVAKPGLSLSELLNVTPTGLFSGDPVIDPKRGLVTFTSKLGVVAVHSRKPTRGKKSKDLLLKMTKPTDDGMPRHVGMVAIDLGQTNPVAAEYSRVVQSDAGTLKQEPVSRGVLPDDLLKDVARYRRAYDLTEESIRQEAIALLSEGHRAEVTKLDQTTANETKRLLVDRGVSESLPWEKMSSNTTYISDCLVALGKTDDVFFVPKAKKGKKETGIAVKRKDHGWSKLLRPRTSPEARKALNENQWAVKRASPEYERLSRRKLELGRRCVNHIIQETKRWTQCEDIVVVLEDLNVGFFHGSGKRPDGWDNFFVSKRENRWFIQVLHKAFGDLATHRGTHVIEVHPARTSITCIKCGHCDAGNRDGESFVCLASACGDRRHADLEVATRNVARVAITGERMPPSEQARDVQKAGGARKRKPSARNVKSSYPAVEPAPASP

SEQ ID NO: 259 - protein, 752 aa, synthetic construct
  source 1..752: note = Description of Artificial Sequence: Synthetic polypeptide
MSDNKMKKLSKEEKPLTPLQILIRKYIDKSQYPSGFKTTIIKQAGVRIKSVKSEQDEINLANWIISKYDPTYIKRDFNPSAKCQIIATSRSVADFDIVKMSNKVQEIFFASSHLDKNVFDIGKSKSDHDSWFERNNVDRGIYTYSNVQGMNLIFSNTKNTYLGVAVKAQNKFSSKMKRIQDINNFRITNHQSPLPIPDEIKIYDDAGFLLNPPGVNPNIFGYQSCLLKPLENKEIISKTSFPEYSRLPADMIEVNYKISNRLKFSNDQKGFIQFKDKLNLFKINSQELFSKRRRLSGQPILLVASFGDDWVVLDGRGLLRQVYYRGIAKPGSITISELLGFFTGDPIVDPIRGVVSLGFKPGVLSQETLKTTSARIFAEKLPNLVLNNNVGLMSIDLGQTNPVSYRLSEITSNMSVEHICSDFLSQDQISSIEKAKTSLDNLEEEIAIKAVDHLSDEDKINFANFSKLNLPEDTRQSLFEKYPELIGSKLDFGSMGSGTSYIADELIKFENKDAFYPSGKKKFDLSFSRDLRKKLSDETRKSYNDALFLEKRTNDKYLKNAKRRKQIVRTVANSLVSKIEELGLTPVINIENLAMSGGFFDGRGKREKGWDNFFKVKKENRWVMKDFHKAFSELSPHHGVIVIESPPYCTSVTCTKCNFCDKKNRNGHKFTCQRCGLDANADLDIATENLEKVAISGKRMPGSERSSDERKVAVARKAKSPKGKAIKGVKCTITDEPALLSANSQDCSQSTS

SEQ ID NO: 260 - protein, 747 aa, synthetic construct
  source 1..747: note = Description of Artificial Sequence: Synthetic polypeptide
MALSLAEVRERHFKGLRFRSSYLKRAGKILKKEGEAACVAYLTGKDEESPPNFKPPAKCDVVAQSRPFEEWPIVQASVAVQSYVYGLTKEAFEAFNPGTTKQSHEACLAATGIDTCGYSNVQGLNLIFRQAKNRYEGVITKVENRNKKAKKKLTRKNEWRQKNGHSELPEAPEELTFNDEGRLLQPPGINPSLYTYQQISPTPWSPKDSSILPPQYAGYERDPNAPIPFGVAKDRLTIASGCPGYIPEWMRTAGEKTNPRTQKKFMHPGLSTRKNKRMRLPRSVRSAPLGALLVTIHLGEDWLVLDVRGLLRNARWRGVAPKDISTQGLLNLFTGDPVIDTRRGVVTFTYKPETVGIHSRTWLYKGKQTKEVLEKLTQDQTVALVAIDLGQTNPVSAAASRVSRSGENLSIETVDRFFLPDELIKELRLYRMAHDRLEERIREESTLALTEAQQAEVRALEHVVRDDAKNKVCAAFNLDAASLPWDQMTSNTTYLSEAILAQGVSRDQVFFTPNPKKGSKEPVEVMRKDRAWVYAFKAKLSEETRKAKNEALWALKRASPDYARLSKRREELCRRSVNMVINRAKKRTQCQVVIPVLEDLNIGFFHGSGKRLPGWDNFFVAKKENRWLMNGLHKSFSDLAVHRGFYVFEVMPHRTSITCPACGHCDSENRDGEAFVCLSCKRTYHADLDVATHNLTQVAGTGLPMPEREHPGGTKKPGGSRKPESPQTHAPILHRTDYSESADRLGS

SEQ ID NO: 261 - protein, 733 aa, synthetic construct
  source 1..733: note = Description of Artificial Sequence: Synthetic polypeptide
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 262 - protein, 702 aa, synthetic construct
  source 1..702: note = Description of Artificial Sequence: Synthetic polypeptide
MIKEQSELSKLIEKYYPGKKFYSNDLKQAGKHLKKSEHLTAKESEELTVEFLKSCKEKLYDFRPPAKALIISTSRPFEEWPIYKASESIQKYIYSLTKEELEKYNISTDKTSQENFFKESLIDNYGFANVSGLNLIFQHTKAIYDGVLKKVNNRNNKILKKYKRKIEEGIEIDSPELEKAIDESGHFINPPGINKNIYCYQQVSPTIFNSFKETKIICPFNYKRNPNDIIQKGVIDRLAIPFGEPGYIPDHQRDKVNKHKKRIRKYYKNNENKNKDAILAKINIGEDWVLFDLRGLLRNAYWRKLIPKQGITPQQLLDMFSGDPVIDPIKNNITFIYKESIIPIHSESIIKTKKSKELLEKLTKDEQIALVSIDLGQTNPVAARFSRLSSDLKPEHVSSSFLPDELKNEICRYREKSDLLEIEIKNKAIKMLSQEQQDEIKLVNDISSEELKNSVCKKYNIDNSKIPWDKMNGFTTFIADEFINNGGDKSLVYFTAKDKKSKKEKLVKLSDKKIANSFKPKISKETREILNKITWDEKISSNEYKKLSKRKLEFARRATNYLINQAKKATRLNNVVLVVEDLNSKFFHGSGKREDGWDNFFIPKKENRWFIQALHKSLTDVSIHRGINVIEVRPERTSITCPKCGCCDKENRKGEDFKCIKCDSVYHADLEVATFNIEKVAITGESMPKPDCERLGGEESIG

SEQ ID NO: 263 - protein, 661 aa, synthetic construct
  source 1..661: note = Description of Artificial Sequence: Synthetic polypeptide
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 264 - protein, 531 aa, synthetic construct
  source 1..531: note = Description of Artificial Sequence: Synthetic polypeptide
LEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGIITFSYKEGVVPVFSQKIVSRFKSRDTLEKLTSQGPVALLSVDLGQNEPVAARVCSLKNINDKIALDNSCRIPFLDDYKKQIKDYRDSLDELEIKIRLEAINSLDVNQQVEIRDLDVFSADRAKASTVDMFDIDPNLISWDSMSDARFSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKSFSELSSNRGLCVIEVNPAWTSATCPDCGFCSKENRDGINFTCRKCGVSYHADIDVATLNIARVAVLGKPMSGPADRERLGGTKKPRVARSRKDMKRKDISNGTVEVMVTA

SEQ ID NO: 265 - protein, 479 aa, synthetic construct
  source 1..479: note = Description of Artificial Sequence: Synthetic polypeptide
IPSFGYLDRLKIAKGQPGYIPEWQRETINPSKKVRRYWATNHEKIRNAIPLVVFIGDDWVIIDGRGLLRDARRRKLADKNTTIEQLLEMVSNDPVIDSTRGIATLSYVEGVVPVRSFIPIGEKKGREYLEKSTQKESVTLLSVDIGQINPVSCGVYKVSNGCSKIDFLDKFFLDKKHLDAIQKYRTLQDSLEASIVNEALDEIDPSFKKEYQNINSQTSNDVKKSLCTEYNIDPEAISWQDITAHSTLISDYLIDNNITNDVYRTVNKAKYKTNDFGWYKKFSAKLSKEAREALNEKIWELKIASSKYKKLSVRKKEIARTIANDCVKRAETYGDNVVVAMESLTKNNKVMSGRGKRDPGWHNLGQAKVENRWFIQAISSAFEDKATHHGTPVLKVNPAYTSQTCPSCGHCSKDNRSSKDRTIFVCKSCGEKFNADLDVATYNIAHVAFSGKKLSPPSEKSSATKKPRSARKSKKSRKS

SEQ ID NO: 266 - protein, 452 aa, synthetic construct
  source 1..452: note = Description of Artificial Sequence: Synthetic polypeptide
SPIEKLLNGLLVKITFGNDWIICDARGLLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 267 - protein, 425 aa, synthetic construct
  source 1..425: note = Description of Artificial Sequence: Synthetic polypeptide
LLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 268 - protein, 735 aa, synthetic construct
  source 1..735: note = Description of Artificial Sequence: Synthetic polypeptide
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 269 - protein, 18 aa, synthetic construct
  source 1..18: note = Description of Artificial Sequence: Synthetic peptide
KRPAATKKAGQAKKKKEF

SEQ ID NO: 270 - RNA, 41 nt, synthetic construct
  source 1..41: mol_type = other RNA; note = Description of Artificial Sequence: Synthetic oligonucleotide
taatttctactaagtgtagatagcattaagtgtcaagttct

SEQ ID NO: 271 - DNA, 240 bp, unidentified
  source 1..240: mol_type = unassigned DNA; note = Description of Unknown: Target nucleic acid sequence
taactctgaaaacatttctagtcttgtaatcaacatcagggtaaaaatcatgtgttaatacaaaggtacaggaacaaagaatttgttcttcatggctctctgtgtctgatccaagaggcgaggccagtttcatttgagcattaagtgtcaagttctgcacgctatcatcatcaggggccgaggcttctctttgtttttaattaattgtttttaactgtgagtttatatacacttgaagca

SEQ ID NO: 272-376 - skipped (105 entries)

SEQ ID NO: 377 - RNA, 41 nt, synthetic construct
  source 1..41: mol_type = other RNA; note = Description of Artificial Sequence: Synthetic oligonucleotide
taatttctactaagtgtagatagctgctcgaattggctttg

SEQ ID NO: 378 - DNA, 360 bp, unidentified
  source 1..360: mol_type = unassigned DNA; note = Description of Unknown: Target nucleic acid sequence
agcagaagcagaggatttgtttagtcactggcaaacaggaaaaaaaaatggcggacaacaacatgaccacaacacaaattgaggtgggtccgggagcaaccaatgccaccataaactttgaagcaggaattctggagtgctatgaaaggctttcatggcaaagggcccttgactaccctggtcaagaccgcctaaacagactaaagagaaaattagagtcaagaataaagactcacaacaaaagtgagcctgaaagtaaaaggatgtctcttgaagagagaaaagcaattggagtaaaaatgatgaaagtactcctatttatgaatccgtctgctggaattgaagggtttgagccatact

SEQ ID NO: 379 - DNA, 12 bp, synthetic construct
  source 1..12: mol_type = other DNA; note = Description of Artificial Sequence: Synthetic oligonucleotide
actttacttgct

SEQ ID NO: 380 - DNA, 690 bp, synthetic construct
  source 1..690: mol_type = other DNA; note = Description of Artificial Sequence: Synthetic polynucleotide
tggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagc

SEQ ID NO: 381 - DNA, 392 bp, synthetic construct
  source 1..392: mol_type = other DNA; note = Description of Artificial Sequence: Synthetic polynucleotide
tctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgca

SEQ ID NO: 382 - DNA, 580 bp, synthetic construct
  source 1..580: mol_type = other DNA; note = Description of Artificial Sequence: Synthetic polynucleotide
ctgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattc

SEQ ID NO: 383 - DNA, 385 bp, synthetic construct
  source 1..385: mol_type = other DNA; note = Description of Artificial Sequence: Synthetic polynucleotide
gacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccaga

SEQ ID NO: 384 - DNA, 672 bp, synthetic construct
  source 1..672: mol_type = other DNA; note = Description of Artificial Sequence: Synthetic polynucleotide
tggtgtttattaccctgacaaagttttcagatcctcagttttacattcaactcaggacttgttcttacctttcttttccaatgttacttggttccatgctatctctgggaccaatggtactaagaggtttgctaaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtggtctccctcagggtttttcggctttagaaccattggtagatttgccaataggtattaacatcactaggtttcaaactttacatataagttatttgactcctggtgattcttcttcaggttggacagctggtgctgcagc

SEQ ID NO: 385 - DNA, 392 bp, synthetic construct
  source 1..392: mol_type = other DNA; note = Description of Artificial Sequence: Synthetic polynucleotide
tctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgca

SEQ ID NO: 386 - DNA, 580 bp, synthetic construct
  source 1..580: mol_type = other DNA; note = Description of Artificial Sequence: Synthetic polynucleotide
ctgagtctaacaaaaagtttctgcctttccaacaatttggcagagacattgatgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcatcggcgggcacgtagtgtagctagtcaatccatcattgcctacactatgtcacttggtgtagaaaattcagttgcttactctaataactctattgccatacccataaattttactattagtgttaccacagaaattctaccagtgtctatgaccaagacatcagtagattgtacaatgtacatttgtggtgattc

SEQ ID NO: 387 - DNA, 385 bp, synthetic construct
  source 1..385: mol_type = other DNA; note = Description of Artificial Sequence: Synthetic polynucleotide
gacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacacacaacacatttgtgtctggtaactgtgatgttgtaataggaattgtcaacaacacagtttatgatcctttgcaacctgaattagactcattcaaggaggagttagataaatattttaagaatcatacatcaccaga

SEQ ID NO: 388 - DNA, 24 bp, synthetic construct
  source 1..24: mol_type = other DNA; note = Description of Artificial Sequence: Synthetic primer
ctgaaatctatcaggccggtagca

SEQ ID NO: 389 - DNA, 26 bp, synthetic construct
  source 1..26: mol_type = other DNA; note = Description of Artificial Sequence: Synthetic primer
gtcaagaatctcaagtgtctgtggat

SEQ ID NO: 390 - DNA, 24 bp, synthetic construct
  source 1..24: mol_type = other DNA; note = Description of Artificial Sequence: Synthetic primer
tgaaatctatcaggccggtagcac

SEQ ID NO: 391 - DNA, 25 bp, synthetic construct
  source 1..25: mol_type = other DNA; note = Description of Artificial Sequence: Synthetic primer
tcaactgaaatctatcaggccggta

SEQ ID NO: 392 - DNA, 22 bp, synthetic construct
  source 1..22: mol_type = other DNA; note = Description of Artificial Sequence: Synthetic primer
atctcaagtgtctgtggatcac

SEQ ID NO: 393 - DNA, 22 bp, synthetic construct
  source 1..22: mol_type = other DNA; note = Description of Artificial Sequence: Synthetic primer
tatcaggccggtagcacacctt

SEQ ID NO: 394 - DNA, 27 bp, synthetic construct
  source 1..27: mol_type = other DNA; note = Description of Artificial Sequence: Synthetic primer
gtgtaatgtcaagaatctcaagtgtct

SEQ ID NO: 395 - DNA, 26 bp, synthetic construct
  source 1..26: mol_type = other DNA; note = Description of Artificial Sequence: Synthetic primer
tgcaggtatatgcgctagttatcaga

SEQ ID NO: 396 - DNA, 28 bp, synthetic construct
  source 1..28: mol_type = other DNA; note = Description of Artificial Sequence: Synthetic primer
gcaacaaaagattgctgcattcagttga

SEQ ID NO: 397 - DNA, 28 bp, synthetic construct
  source 1..28: mol_type = other DNA; note = Description of Artificial Sequence: Synthetic primer
caggtatatgcgctagttatcagactca

SEQ ID NO: 398 - DNA, 26 bp, synthetic construct
  source 1..26: mol_type = other DNA; note = Description of Artificial Sequence: Synthetic primer
ggtgcaggtatatgcgctagttatca

SEQ ID NO: 399 - DNA, 26 bp, synthetic construct
  source 1..26: mol_type = other DNA; note = Description of Artificial Sequence: Synthetic primer
attggtgcaggtatatgcgctagtta

SEQ ID NO: 400 - DNA, 27 bp, synthetic construct
  source 1..27: mol_type = other DNA; note = Description of Artificial Sequence: Synthetic primer
aggttttaattgttactttcctttaca

SEQ ID NO: 401 - DNA, 27 bp, synthetic construct
  source 1..27: mol_type = other DNA; note = Description of Artificial Sequence: Synthetic primer
gctggtgcatgtagaagttcaaaagaa

SEQ ID NO: 402 - DNA, 26 bp, synthetic construct
  source 1..26: mol_type = other DNA; note = Description of Artificial Sequence: Synthetic primer
ttgtaattagaggtgatgaagtcaga

SEQ ID NO: 403 - DNA, 27 bp, synthetic construct
  source 1..27: mol_type = other DNA; note = Description of Artificial Sequence: Synthetic primer
attccaagctataacgcagcctgtaaa

SEQ ID NO: 404 - DNA, 27 bp, synthetic construct
  source 1..27: mol_type = other DNA; note = Description of Artificial Sequence: Synthetic primer
tgtaattagaggtgatgaagtcagaca

SEQ ID NO: 405 - DNA, 27 bp, synthetic construct
  source 1..27: mol_type = other DNA; note = Description of Artificial Sequence: Synthetic primer
gaattccaagctataacgcagcctgta

SEQ ID NO: 406 - DNA, 27 bp, synthetic construct
  source 1..27: mol_type = other DNA; note = Description of Artificial Sequence: Synthetic primer
attgttaataacgctactaatgttgtt

SEQ ID NO: 407 - DNA, 25 bp, synthetic construct
  source 1..25: mol_type = other DNA; note = Description of Artificial Sequence: Synthetic primer
cactttccatccaacttttgttgtt

SEQ ID NO: 408 - DNA, 26 bp, synthetic construct
  source 1..26: mol_type = other DNA; note = Description of Artificial Sequence: Synthetic primer
aacgctactaatgttgttattaaagt

SEQ ID NO: 409 - DNA, 27 bp, synthetic construct
  source 1..27: mol_type = other DNA; note = Description of Artificial Sequence: Synthetic primer
actctgaactcactttccatccaactt

SEQ ID NO: 410 - DNA, 25 bp, synthetic construct
  source 1..25: mol_type = other DNA; note = Description of Artificial Sequence: Synthetic primer
aatgatggattgactagctacacta

SEQ ID NO: 411 - DNA, 27 bp, synthetic construct
  source 1..27: mol_type = other DNA; note = Description of Artificial Sequence: Synthetic primer
aggtgttcttactgagtctaacaaaaa

SEQ ID NO: 412 - DNA, 25 bp, synthetic construct
  source 1..25: mol_type = other DNA; note = Description of Artificial Sequence: Synthetic primer
gtcaagaatctcaagtgtctgtgga

SEQ ID NO: 413 - DNA, 26 bp, synthetic construct
  source 1..26: mol_type = other DNA; note = Description of Artificial Sequence: Synthetic primer
aggcacaggtgttcttactgagtcta

SEQ ID NO: 414 - DNA, 23 bp, synthetic construct
  source 1..23: mol_type = other DNA; note = Description of Artificial Sequence: Synthetic primer
ctcaagtgtctgtggatcacgga

SEQ ID NO: 415 - DNA, 27 bp, synthetic construct
  source 1..27: mol_type = other DNA; note = Description of Artificial Sequence: Synthetic primer
cagttttacattcaactcaggacttgt

SEQ ID NO: 416 - DNA, 27 bp, synthetic construct
  source 1..27: mol_type = other DNA; note = Description of Artificial Sequence: Synthetic primer
tatgttagacttctcagtggaagcaaa

SEQ ID NO: 417 - DNA, 27 bp, synthetic construct
  source 1..27: mol_type = other DNA; note = Description of Artificial Sequence: Synthetic primer
agttttacattcaactcaggacttgtt

SEQ ID NO: 418 - DNA, 24 bp, synthetic construct
  source 1..24: mol_type = other DNA; note = Description of Artificial Sequence: Synthetic primer
tgttagacttctcagtggaagcaa

SEQ ID NO: 419 - DNA, 27 bp, synthetic construct
  source 1..27: mol_type = other DNA; note = Description of Artificial Sequence: Synthetic primer
gttttacattcaactcaggacttgttc

SEQ ID NO: 420 - DNA, 22 bp, synthetic construct
  source 1..22: mol_type = other DNA; note = Description of Artificial Sequence: Synthetic primer
gttagacttctcagtggaagca

SEQ ID NO: 421 - DNA, 25 bp, synthetic construct
  source 1..25: mol_type = other DNA; note = Description of Artificial Sequence: Synthetic primer
gacatacccattggtgcaggtatat

SEQ ID NO: 422 - DNA, 26 bp, synthetic construct
  source 1..26: mol_type = other DNA; note = Description of Artificial Sequence: Synthetic primer
gtgggtatggcaatagagttattaga

SEQ ID NO: 423 - DNA, 24 bp, synthetic construct
  source 1..24: mol_type = other DNA; note = Description of Artificial Sequence: Synthetic primer
gacatacccattggtgcaggtata

SEQ ID NO: 424 - DNA, 27 bp, synthetic construct
  source 1..27: mol_type = other DNA; note = Description of Artificial Sequence: Synthetic primer
gtgggtatggcaatagagttattagag

SEQ ID NO: 425 - DNA, 23 bp, synthetic construct
  source 1..23: mol_type = other DNA; note = Description of Artificial Sequence: Synthetic primer
tgacatacccattggtgcaggta

SEQ ID NO: 426 - DNA, 27 bp, synthetic construct
  source 1..27: mol_type = other DNA; note = Description of Artificial Sequence: Synthetic primer
gggtatggcaatagagttattagagta

SEQ ID NO: 427 - RNA, 41 nt, synthetic construct
  source 1..41: mol_type = other RNA; note = Description of Artificial Sequence: Synthetic oligonucleotide
taatttctactaagtgtagatatgctgtctctgggaccaat

SEQ ID NO: 428 - RNA, 41 nt, synthetic construct
  source 1..41: mol_type = other RNA; note = Description of Artificial Sequence: Synthetic oligonucleotide
taatttctactaagtgtagattgctgtctctgggaccaatg

SEQ ID NO: 429 - RNA, 41 nt, synthetic construct
  source 1..41: mol_type = other RNA; note = Description of Artificial Sequence: Synthetic oligonucleotide
taatttctactaagtgtagatataaccctgtcctaccattt

SEQ ID NO: 430 - RNA, 41 nt, synthetic construct
  source 1..41: mol_type = other RNA; note = Description of Artificial Sequence: Synthetic oligonucleotide
taatttctactaagtgtagatctaaccctgtcctaccattt

SEQ ID NO: 431 - RNA, 41 nt, synthetic construct
  source 1..41: mol_type = other RNA; note = Description of Artificial Sequence: Synthetic oligonucleotide
taatttctactaagtgtagatctaaccctatcctaccattt

SEQ ID NO: 432 - RNA, 41 nt, synthetic construct
  source 1..41: mol_type = other RNA; note = Description of Artificial Sequence: Synthetic oligonucleotide
taatttctactaagtgtagattcaaacctcttagtaccatt

SEQ ID NO: 433 - RNA, 41 nt, synthetic construct
  source 1..41: mol_type = other RNA; note = Description of Artificial Sequence: Synthetic oligonucleotide
taatttctactaagtgtagatgcaaacctcttagtaccatt

SEQ ID NO: 434 - RNA, 41 nt, synthetic construct
  source 1..41: mol_type = other RNA; note = Description of Artificial Sequence: Synthetic oligonucleotide
taatttctactaagtgtagatgcaaacttcttagtaccatt

SEQ ID NO: 435 - RNA, 41 nt, synthetic construct
  source 1..41: mol_type = other RNA; note = Description of Artificial Sequence: Synthetic oligonucleotide
taatttctactaagtgtagattaatggtgttgaaggtttta

SEQ ID NO: 436 - RNA, 41 nt, synthetic construct
  source 1..41: mol_type = other RNA; note = Description of Artificial Sequence: Synthetic oligonucleotide
taatttctactaagtgtagattaatggtgttaaaggtttta

SEQ ID NO: 437 - RNA, 41 nt, synthetic construct
  source 1..41: mol_type = other RNA; note = Description of Artificial Sequence: Synthetic oligonucleotide
taatttctactaagtgtagattaatgatgttaaaggtttta

SEQ ID NO: 438 - RNA, 41 nt, synthetic construct
  source 1..41: mol_type = other RNA; note = Description of Artificial Sequence: Synthetic oligonucleotide
taatttctactaagtgtagatgtaatggtgttgaaggtttt

SEQ ID NO: 439 - RNA, 41 nt, synthetic construct
  source 1..41: mol_type = other RNA; note = Description of Artificial Sequence: Synthetic oligonucleotide
taatttctactaagtgtagatgtaatggtgttaaaggtttt

SEQ ID NO: 440 - RNA, 41 nt, synthetic construct
  source 1..41: mol_type = other RNA; note = Description of Artificial Sequence: Synthetic oligonucleotide
taatttctactaagtgtagatgtaatgatgttaaaggtttt

SEQ ID NO: 441 - RNA, 41 nt, synthetic construct
  source 1..41: mol_type = other RNA; note = Description of Artificial Sequence: Synthetic oligonucleotide
taatttctactaagtgtagataaaccttcaacaccattaca

SEQ ID NO: 442 - RNA, 41 nt, synthetic construct
  source 1..41: mol_type = other RNA; note = Description of Artificial Sequence: Synthetic oligonucleotide
taatttctactaagtgtagataaacctttaacaccattaca

SEQ ID NO: 443 - RNA, 41 nt, synthetic construct
  source 1..41: mol_type = other RNA; note = Description of Artificial Sequence: Synthetic oligonucleotide
taatttctactaagtgtagataaacctttaacatcattaca

SEQ ID NO: 444 - RNA, 41 nt, synthetic construct
  source 1..41: mol_type = other RNA; note = Description of Artificial Sequence: Synthetic oligonucleotide
taatttctactaagtgtagatcaacccactaatggtgttgg

SEQ ID NO: 445 - RNA, 41 nt, synthetic construct
  source 1..41: mol_type = other RNA; note = Description of Artificial Sequence: Synthetic oligonucleotide
taatttctactaagtgtagatcaacccacttatggtgttgg

SEQ ID NO: 446 - RNA, 41 nt, synthetic construct
  source 1..41: mol_type = other RNA; note = Description of Artificial Sequence: Synthetic oligonucleotide
taatttctactaagtgtagatcaaccctcttatggtgttgg

SEQ ID NO: 447 - RNA, 41 nt, synthetic construct
  source 1..41: mol_type = other RNA; note = Description of Artificial Sequence: Synthetic oligonucleotide
taatttctactaagtgtagataacccactaatggtgttggt

SEQ ID NO: 448 - RNA, 41 nt, synthetic construct
  source 1..41: mol_type = other RNA; note = Description of Artificial Sequence: Synthetic oligonucleotide
taatttctactaagtgtagataacccacttatggtgttggt

SEQ ID NO: 449 - RNA, 41 nt, synthetic construct
  source 1..41: mol_type = other RNA; note = Description of Artificial Sequence: Synthetic oligonucleotide
taatttctactaagtgtagataaccctcttatggtgttggt

SEQ ID NO: 450 - RNA, 41 nt, synthetic construct
  source 1..41: mol_type = other RNA; note = Description of Artificial Sequence: Synthetic oligonucleotide
taatttctactaagtgtagatacccactaatggtgttggtt

SEQ ID NO: 451 - RNA, 41 nt, synthetic construct
  source 1..41: mol_type = other RNA; note = Description of Artificial Sequence: Synthetic oligonucleotide
taatttctactaagtgtagatacccacttatggtgttggtt

SEQ ID NO: 452 - RNA, 41 nt, synthetic construct
  source 1..41: mol_type = other RNA; note = Description of Artificial Sequence: Synthetic oligonucleotide
taatttctactaagtgtagataccctcttatggtgttggtt

SEQ ID NO: 453 - RNA, 41 nt, synthetic construct
  source 1..41: mol_type = other RNA; note = Description of Artificial Sequence: Synthetic oligonucleotide
taatttctactaagtgtagatgtaaccaacaccattagtgg

SEQ ID NO: 454 - RNA, 41 nt, synthetic construct
  source 1..41: mol_type = other RNA; note = Description of Artificial Sequence: Synthetic oligonucleotide
taatttctactaagtgtagatgtaaccaacaccataagtgg

SEQ ID NO: 455 - RNA, 41 nt, synthetic construct
  source 1..41: mol_type = other RNA; note = Description of Artificial Sequence: Synthetic oligonucleotide
taatttctactaagtgtagatgtaaccgacaccataagtgg

SEQ ID NO: 456 - RNA, 41 nt, synthetic construct
  source 1..41: mol_type = other RNA; note = Description of Artificial Sequence: Synthetic oligonucleotide
taatttctactaagtgtagatgcagagacattgctgacact

SEQ ID NO: 457 - RNA, 41 nt, synthetic construct
  source 1..41: mol_type = other RNA; note = Description of Artificial Sequence: Synthetic oligonucleotide
taatttctactaagtgtagatgcagagacattgatgacact

SEQ ID NO: 458 - RNA, 41 nt, synthetic construct
  source 1..41: mol_type = other RNA; note = Description of Artificial Sequence: Synthetic oligonucleotide
taatttctactaagtgtagatgcagaggcattgatgacact

SEQ ID NO: 459 - RNA, 41 nt, synthetic construct
  source 1..41: mol_type = other RNA; note = Description of Artificial Sequence: Synthetic oligonucleotide
taatttctactaagtgtagatctgacactactgatgctgtc

SEQ ID NO: 460 - RNA, 41 nt, synthetic construct
  source 1..41: mol_type = other RNA; note = Description of Artificial Sequence: Synthetic oligonucleotide
taatttctactaagtgtagatatgacactactgatgctgtc

SEQ ID NO: 461 - RNA, 41 nt, synthetic construct
  source 1..41: mol_type = other RNA; note = Description of Artificial Sequence: Synthetic oligonucleotide
taatttctactaagtgtagatatgacattactgatgctgtc

SEQ ID NO: 462 - RNA, 41 nt, synthetic construct
  source 1..41: mol_type = other RNA; note = Description of Artificial Sequence: Synthetic oligonucleotide
taatttctactaagtgtagatgtagtgtcagcaatgtctct

SEQ ID NO: 463 - RNA, 41 nt, synthetic construct
  source 1..41: mol_type = other RNA; note = Description of Artificial Sequence: Synthetic oligonucleotide
taatttctactaagtgtagatgtagtgtcatcaatgtctct

SEQ ID NO: 464 - RNA, 41 nt, synthetic construct
  source 1..41: mol_type = other RNA; note = Description of Artificial Sequence: Synthetic oligonucleotide
taatttctactaagtgtagatgtagtgccatcaatgtctct

SEQ ID NO: 465 - RNA, 41 nt, synthetic construct
  source 1..41: mol_type = other RNA; note = Description of Artificial Sequence: Synthetic oligonucleotide
taatttctactaagtgtagatcctcggcgggcacgtagtgt

SEQ ID NO: 466 - RNA, 41 nt, synthetic construct
  source 1..41: mol_type = other RNA; note = Description of Artificial Sequence: Synthetic oligonucleotide
taatttctactaagtgtagatcatcggcgggcacgtagtgt

SEQ ID NO: 467 - RNA, 41 nt, synthetic construct
  source 1..41: mol_type = other RNA; note = Description of Artificial Sequence: Synthetic oligonucleotide
taatttctactaagtgtagatcatcggtgggcacgtagtgt

SEQ ID NO: 468 - RNA, 41 nt, synthetic construct
  source 1..41: mol_type = other RNA; note = Description of Artificial Sequence: Synthetic oligonucleotide
taatttctactaagtgtagatgtgcagaaaattcagttgct

SEQ ID NO: 469 - RNA, 41 nt, synthetic construct
  source 1..41: mol_type = other RNA; note = Description of Artificial Sequence: Synthetic oligonucleotide
taatttctactaagtgtagatgtgtagaaaattcagttgct

SEQ ID NO: 470 - RNA, 41 nt, synthetic construct
  source 1..41: mol_type = other RNA; note = Description of Artificial Sequence: Synthetic oligonucleotide
taatttctactaagtgtagatgtgtaggaaattcagttgct

SEQ ID NO: 471 - RNA, 41 nt, synthetic construct
  source 1..41: mol_type = other RNA; note = Description of Artificial Sequence: Synthetic oligonucleotide
taatttctactaagtgtagattgcaccaagtgacatagtgt

SEQ ID NO: 472 - RNA, 41 nt, synthetic construct
  source 1..41: mol_type = other RNA; note = Description of Artificial Sequence: Synthetic oligonucleotide
taatttctactaagtgtagattacaccaagtgacatagtgt

SEQ ID NO: 473 - RNA, 41 nt, synthetic construct
  source 1..41: mol_type = other RNA; note = Description of Artificial Sequence: Synthetic oligonucleotide
taatttctactaagtgtagattacaccaaatgacatagtgt

SEQ ID NO: 474 - RNA, 41 nt, synthetic construct
  source 1..41: mol_type = other RNA; note = Description of Artificial Sequence: Synthetic oligonucleotide
taatttctactaagtgtagatttgccatacccacaaatttt

SEQ ID NO: 475 - RNA, 41 nt, synthetic construct
  source 1..41: mol_type = other RNA; note = Description of Artificial Sequence: Synthetic oligonucleotide
taatttctactaagtgtagatttgccatacccataaatttt

SEQ ID NO: 476 - RNA, 41 nt, synthetic construct
  source 1..41: mol_type = other RNA; note = Description of Artificial Sequence: Synthetic oligonucleotide
taatttctactaagtgtagatttgccacacccataaatttt

SEQ ID NO: 477 - RNA, 41 nt, synthetic construct
  source 1..41: mol_type = other RNA; note = Description of Artificial Sequence: Synthetic oligonucleotide
taatttctactaagtgtagatattgccatacccacaaattt

SEQ ID NO: 478 - RNA, 41 nt, synthetic construct
  source 1..41: mol_type = other RNA; note = Description of Artificial Sequence: Synthetic oligonucleotide
taatttctactaagtgtagatattgccatacccataaattt

SEQ ID NO: 479 - RNA, 41 nt, synthetic construct
  source 1..41: mol_type = other RNA; note = Description of Artificial Sequence: Synthetic oligonucleotide
taatttctactaagtgtagatattgccgtacccataaattt

SEQ ID NO: 480 - RNA, 41 nt, synthetic construct
  source 1..41: mol_type = other RNA; note = Description of Artificial Sequence: Synthetic oligonucleotide
taatttctactaagtgtagatccatacccacaaattttact

SEQ ID NO: 481 - RNA, 41 nt, synthetic construct
  source 1..41: mol_type = other RNA; note = Description of Artificial Sequence: Synthetic oligonucleotide
taatttctactaagtgtagatccatacccataaattttact

SEQ ID NO: 482 - DNA, 41 bp, synthetic construct
  source 1..41: mol_type = other DNA; note = Description of Artificial Sequence: Synthetic oligonucleotide; note = Description of Combined DNA/RNA Molecule: Synthetic oligonucleotide
  modified_base 41: mod_base = OTHER; note = thymine
taatttctactaagtgtagatccatactcataaattttact

SEQ ID NO: 483 - DNA, 20 bp, synthetic construct
  source 1..20: mol_type = other DNA; note = Description of Artificial Sequence: Synthetic oligonucleotide
atgctgtctctgggaccaat

SEQ ID NO: 484 - DNA, 20 bp, synthetic construct
  source 1..20: mol_type = other DNA; note = Description of Artificial Sequence: Synthetic oligonucleotide
tgctgtctctgggaccaatg

SEQ ID NO: 485 - DNA, 20 bp, Severe acute respiratory syndrome coronavirus 2
  source 1..20: mol_type = unassigned DNA
ataaccctgtcctaccattt

SEQ ID NO: 486 - DNA, 20 bp, synthetic construct
  source 1..20: mol_type = other DNA; note = Description of Artificial Sequence: Synthetic oligonucleotide
ctaaccctgtcctaccattt

SEQ ID NO: 487 - DNA, 20 bp, synthetic construct
  source 1..20: mol_type = other DNA; note = Description of Artificial Sequence: Synthetic oligonucleotide
ctaaccctatcctaccattt

SEQ ID NO: 488 - DNA, 20 bp, Severe acute respiratory syndrome coronavirus 2
  source 1..20: mol_type = unassigned DNA
tcaaacctcttagtaccatt

SEQ ID NO: 489 - DNA, 20 bp, synthetic construct
  source 1..20: mol_type = other DNA; note = Description of Artificial Sequence: Synthetic oligonucleotide
gcaaacctcttagtaccatt

SEQ ID NO: 490 - DNA, 20 bp, synthetic construct
  source 1..20: mol_type = other DNA; note = Description of Artificial Sequence: Synthetic oligonucleotide
gcaaacttcttagtaccatt

SEQ ID NO: 491 - DNA, 20 bp, Severe acute respiratory syndrome coronavirus 2
  source 1..20: mol_type = unassigned DNA
taatggtgttgaaggtttta

SEQ ID NO: 492 - DNA, 20 bp, synthetic construct
  source 1..20: mol_type = other DNA; note = Description of Artificial Sequence: Synthetic oligonucleotide
taatggtgttaaaggtttta

SEQ ID NO: 493 - DNA, 20 bp, synthetic construct
  source 1..20: mol_type = other DNA; note = Description of Artificial Sequence: Synthetic oligonucleotide
taatgatgttaaaggtttta

SEQ ID NO: 494 - DNA, 20 bp, Severe acute respiratory syndrome coronavirus 2
  source 1..20: mol_type = unassigned DNA
gtaatggtgttgaaggtttt

SEQ ID NO: 495 - DNA, 20 bp, synthetic construct
  source 1..20: mol_type = other DNA; note = Description of Artificial Sequence: Synthetic oligonucleotide
gtaatggtgttaaaggtttt

SEQ ID NO: 496 - DNA, 20 bp, synthetic construct
  source 1..20: mol_type = other DNA; note = Description of Artificial Sequence: Synthetic oligonucleotide
gtaatgatgttaaaggtttt

SEQ ID NO: 497 - DNA, 20 bp, Severe acute respiratory syndrome coronavirus 2
  source 1..20: mol_type = unassigned DNA
aaaccttcaacaccattaca

SEQ ID NO: 498 - DNA, 20 bp, synthetic construct
  source 1..20: mol_type = other DNA; note = Description of Artificial Sequence: Synthetic oligonucleotide
aaacctttaacaccattaca

SEQ ID NO: 499 - DNA, 20 bp, synthetic construct
  source 1..20: mol_type = other DNA; note = Description of Artificial Sequence: Synthetic oligonucleotide
aaacctttaacatcattaca

SEQ ID NO: 500 - DNA, 20 bp, Severe acute respiratory syndrome coronavirus 2
  source 1..20: mol_type = unassigned DNA
caacccactaatggtgttgg

SEQ ID NO: 501 - DNA, 20 bp, synthetic construct
  source 1..20: mol_type = other DNA; note = Description of Artificial Sequence: Synthetic oligonucleotide
caacccacttatggtgttgg

SEQ ID NO: 502 - DNA, 20 bp, synthetic construct
  source 1..20: mol_type = other DNA; note = Description of Artificial Sequence: Synthetic oligonucleotide
caaccctcttatggtgttgg

SEQ ID NO: 503 - DNA, 20 bp, Severe acute respiratory syndrome coronavirus 2
  source 1..20: mol_type = unassigned DNA
aacccactaatggtgttggt

SEQ ID NO: 504 - DNA, 20 bp, synthetic construct
  source 1..20: mol_type = other DNA; note = Description of Artificial Sequence: Synthetic oligonucleotide
aacccacttatggtgttggt

SEQ ID NO: 505 - DNA, 20 bp, synthetic construct
  source 1..20: mol_type = other DNA; note = Description of Artificial Sequence: Synthetic oligonucleotide
aaccctcttatggtgttggt

SEQ ID NO: 506 - DNA, 20 bp, Severe acute respiratory syndrome coronavirus 2
  source 1..20: mol_type = unassigned DNA
acccactaatggtgttggtt

SEQ ID NO: 507 - DNA, 20 bp, synthetic construct
  source 1..20: mol_type = other DNA; note = Description of Artificial Sequence: Synthetic oligonucleotide
acccacttatggtgttggtt

SEQ ID NO: 508 - DNA, 20 bp, synthetic construct
  source 1..20: mol_type = other DNA; note = Description of Artificial Sequence: Synthetic oligonucleotide
accctcttatggtgttggtt

SEQ ID NO: 509 - DNA, 20 bp, Severe acute respiratory syndrome coronavirus 2
  source 1..20: mol_type = unassigned DNA
gtaaccaacaccattagtgg

SEQ ID NO: 510 - DNA, 20 bp, synthetic construct
  source 1..20: mol_type = other DNA; note = Description of Artificial Sequence: Synthetic oligonucleotide
gtaaccaacaccataagtgg

SEQ ID NO: 511 - DNA, 20 bp, synthetic construct
  source 1..20: mol_type = other DNA; note = Description of Artificial Sequence: Synthetic oligonucleotide
gtaaccgacaccataagtgg

SEQ ID NO: 512 - DNA, 20 bp, Severe acute respiratory syndrome coronavirus 2
  source 1..20: mol_type = unassigned DNA
gcagagacattgctgacact

SEQ ID NO: 513 - DNA, 20 bp, synthetic construct
  source 1..20: mol_type = other DNA; note = Description of Artificial Sequence: Synthetic oligonucleotide
gcagagacattgatgacact

SEQ ID NO: 514 - DNA, 20 bp, synthetic construct
  source 1..20: mol_type = other DNA; note = Description of Artificial Sequence: Synthetic oligonucleotide
gcagaggcattgatgacact

SEQ ID NO: 515 - DNA, 20 bp, Severe acute respiratory syndrome coronavirus 2
  source 1..20: mol_type = unassigned DNA
ctgacactactgatgctgtc

SEQ ID NO: 516 - DNA, 20 bp, synthetic construct
  source 1..20: mol_type = other DNA; note = Description of Artificial Sequence: Synthetic oligonucleotide
atgacactactgatgctgtc

SEQ ID NO: 517 - DNA, 20 bp, synthetic construct
  source 1..20: mol_type = other DNA; note = Description of Artificial Sequence: Synthetic oligonucleotide
atgacattactgatgctgtc

SEQ ID NO: 518 - DNA, 20 bp, Severe acute respiratory syndrome coronavirus 2
  source 1..20: mol_type = unassigned DNA
ccatacccacaaattttact

SEQ ID NO: 519 - DNA, 20 bp, synthetic construct
  source 1..20: mol_type = other DNA; note = Description of Artificial Sequence: Synthetic oligonucleotide
ccatacccataaattttact

SEQ ID NO: 520 - DNA, 20 bp, synthetic construct
  source 1..20: mol_type = other DNA; note = Description of Artificial Sequence: Synthetic oligonucleotide
ccatactcataaattttact

SEQ ID NO: 521 - DNA, 20 bp, synthetic construct
  source 1..20: mol_type = other DNA; note = Description of Artificial Sequence: Synthetic oligonucleotide
ccatacccataaattttact

SEQ ID NO: 522 - DNA, 20 bp, synthetic construct
  source 1..20: mol_type = other DNA; note = Description of Artificial Sequence: Synthetic oligonucleotide
ccatactcataaattttact

SEQ ID NO: 523 - DNA, 20 bp, synthetic construct
  source 1..20: mol_type = other DNA; note = Description of Artificial Sequence: Synthetic primer
cctccgtgatatggctcttc

SEQ ID NO: 524 - DNA, 18 bp, synthetic construct
  source 1..18: mol_type = other DNA; note = Description of Artificial Sequence: Synthetic primer
agagtcctttgggcttcc

SEQ ID NO: 525 - DNA, 25 bp, synthetic construct
  source 1..25: mol_type = other DNA; note = Description of Artificial Sequence: Synthetic primer
aggtgcctggaatattggtgaacag

SEQ ID NO: 526 - DNA, 26 bp, synthetic construct
  source 1..26: mol_type = other DNA; note = Description of Artificial Sequence: Synthetic primer
tcaagagtgcgggagaaaattgatcg

SEQ ID NO: 527 - RNA, 41 nt, synthetic construct
  source 1..41: mol_type = other RNA; note = Description of Artificial Sequence: Synthetic oligonucleotide
taatttctactaagtgtagatcatcagaggctgctcgtgtt

SEQ ID NO: 528 - DNA, 122 bp, Severe acute respiratory syndrome coronavirus 2
  source 1..122: mol_type = unassigned DNA
gaccaggaactaatcagacaaggaactgattacaaacattggccgcaaattgcacaatttgcccccagcgcttcagcgttcttcggaatgtcgcgcattggcatggaagtcacaccttcggg

SEQ ID NO: 529 - DNA, 122 bp, unidentified
  source 1..122: mol_type = unassigned DNA; note = Description of Unknown: Bat-SL sequence
gaccaagaattaatcagacaaggaactgattacaaacattggccgcaaattgcacaatttgctccaagtgcctctgcattctttggaatgtcacgcattggcatggaagtcacaccttcggg

SEQ ID NO: 530 - DNA, 122 bp, unidentified
  source 1..122: mol_type = unassigned DNA; note = Description of Unknown: Bat-SL sequence
gaccaagaattaatcagacaaggaactgattacaaacattggccgcaaattgcacaatttgctccaagtgcctctgcattctttggaatgtctcgcattggcatggaagtcacaccttcggg

SEQ ID NO: 531 - DNA, 122 bp, unidentified
  source 1..122: mol_type = unassigned DNA; note = Description of Unknown: SARS-CoV sequence
gaccaagacctaatcagacaaggaactgattacaaacattggccgcaaattgcacaatttgctccaagtgcctctgcattctttggaatgtcacgcattggcatggaagtcacaccttcggg

SEQ ID NO: 532 - DNA, 133 bp, Severe acute respiratory syndrome coronavirus 2
  source 1..133: mol_type = unassigned DNA
gatcacattggcacccgcaatcctgctaacaatgctgcaatcgtgctacaacttcctcaaggaacaacattgccaaaaggcttctacgcagaagggagcagaggcggcagtcaagcctcttctcgttcctcat

SEQ ID NO: 533 - DNA, 133 bp, unidentified
  source 1..133: mol_type = unassigned DNA; note = Description of Unknown: Bat-SL sequence
gaccacattggcacccgcaatcctgctaacaatgctgcaatcgtgctacaacttcctcaaggaacaacattgccaaaaggcttctacgcagaagggagcagaggcggcagtcaagcttcttcacgctcctcat

SEQ ID NO: 534 - DNA, 133 bp, unidentified
  source 1..133: mol_type = unassigned DNA; note = Description of Unknown: SARS-CoV sequence
gaccacattggcacccgcaatcctaataacaatgctgccaccgtgctacaacttcctcaaggaacaacattgccaaaaggcttctacgcagagggaagcagaggcggcagtcaagcctcttctcgctcctcat

SEQ ID NO: 535 - DNA, 119 bp, Severe acute respiratory syndrome coronavirus 2
  source 1..119: mol_type = unassigned DNA
agagacaggtacgttaatagttaatagcgtacttctttttcttgctttcgtggtattcttgctagttacactagccatccttactgcgcttcgattgtgtgcgtactgctgcaatattg

SEQ ID NO: 536 - DNA, 119 bp, unidentified
  source 1..119: mol_type = unassigned DNA; note = Description of Unknown: Bat-SL sequence
agagacaggtacgttaatagttaatagcgtacttctttttcttgcttttgtggtattcttgctagtcacactagccatccttactgcgcttcgattgtgtgcgtactgctgcaatattg

SEQ ID NO: 537 - DNA, 119 bp, unidentified
  source 1..119: mol_type = unassigned DNA; note = Description of Unknown: SARS-CoV sequence
agaaacaggtacgttaatagttaatagcgtacttctttttcttgctttcgtggtattcttgctagtcacactagccatccttactgcgcttcgattgtgtgcgtactgctgcaatattg

SEQ ID NO: 538 - DNA, 34 bp, Severe acute respiratory syndrome coronavirus 2
  source 1..34: mol_type = unassigned DNA
cacaatttgcccccagcgcttcagcgttcttcgg

SEQ ID NO: 539 - DNA, 34 bp, unidentified
  source 1..34: mol_type = unassigned DNA; note = Description of Unknown: Bat-SL-CoVZC45 sequence
cacaatttgctccaagtgcctctgcattctttgg

SEQ ID NO: 540 - DNA, 34 bp, unidentified; identical to SEQ ID NO: 539
  source 1..34: mol_type = unassigned DNA; note = Description of Unknown: SARS-CoV sequence

SEQ ID NO: 541 - DNA, 34 bp, Severe acute respiratory syndrome coronavirus 2
  source 1..34: mol_type = unassigned DNA
agccttttggcaatgttgttccttgaggaagttg

SEQ ID NO: 542 - DNA, 34 bp, unidentified; identical to SEQ ID NO: 541
  source 1..34: mol_type = unassigned DNA; note = Description of Unknown: Bat-SL-CoVZC45 sequence

SEQ ID NO: 543 - DNA, 34 bp, unidentified; identical to SEQ ID NO: 541
  source 1..34: mol_type = unassigned DNA; note = Description of Unknown: SARS-CoV sequence

SEQ ID NO: 544 - DNA, 34 bp, Severe acute respiratory syndrome coronavirus 2
  source 1..34: mol_type = unassigned DNA
ttctttttcttgctttcgtggtattcttgctagt

SEQ ID NO: 545 - DNA, 34 bp, unidentified
  source 1..34: mol_type = unassigned DNA; note = Description of Unknown: Bat-SL-CoVZC45 sequence
ttctttttcttgcttttgtggtattcttgctagt

SEQ ID NO: 546 - DNA, 34 bp, unidentified; identical to SEQ ID NO: 544
  source 1..34: mol_type = unassigned DNA; note = Description of Unknown: SARS-CoV sequence

SEQ ID NO: 547 - DNA, 34 bp, Severe acute respiratory syndrome coronavirus 2
  source 1..34: mol_type = unassigned DNA
cttgctttcgtggtattcttgctagttacactag

SEQ ID NO: 548 - DNA, 34 bp, unidentified
  source 1..34: mol_type = unassigned DNA; note = Description of Unknown: Bat-SL-CoVZC45 sequence
cttgctttcgtggtattcttgctagtcacactag

SEQ ID NO: 549 - DNA, 34 bp, unidentified; identical to SEQ ID NO: 548
  source 1..34: mol_type = unassigned DNA; note = Description of Unknown: SARS-CoV sequence

SEQ ID NO: 550 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 551 - DNA, 3822 bp, Severe acute respiratory syndrome coronavirus 2
  source 1..3822: mol_type = unassigned DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 552 - DNA, 35 bp, synthetic construct
  source 1..35: mol_type = other DNA; note = Description of Artificial Sequence: Synthetic oligonucleotide
caccttgtaatggtgttgaaggttttaattgttac

SEQ ID NO: 553 - protein, 12 aa, synthetic construct
  source 1..12: note = Description of Artificial Sequence: Synthetic peptide
TPCNGVEGFNCY

SEQ ID NO: 554 - DNA, 42 bp, synthetic construct
  source 1..42: mol_type = other DNA; note = Description of Artificial Sequence: Synthetic oligonucleotide
tcatatggtttccaacccactaatggtgttggttaccaacca

SEQ ID NO: 555 - protein, 14 aa, synthetic construct
  source 1..14: note = Description of Artificial Sequence: Synthetic peptide
SYGFQPTNGVGYQP

(19) United States
(12) Patent Application Publication
(10) Pub. No.: US 2023/0332253 A1
(43) Pub. Date: Oct. 19, 2023

(54) COMPOSITIONS AND METHODS FOR DETECTION OF CORONAVIRUS

(71) Applicant: Mammoth Biosciences, Inc., Brisbane, CA (US)

(72) Inventors: Maria-Nefeli TSALOGLOU, San Francisco, CA (US); Clare FASCHING, Redwood City, CA (US); Jesus CHING, Saratoga, CA (US); Janice Sha CHEN, San Francisco, CA (US); James Paul BROUGHTON, San Francisco, CA (US); Daniel Thomas DRZAL, Pacifica, CA (US); Sarah Jane SHAPIRO, Pacifica, CA (US)

(21) Appl. No.: 17/817,889

(22) Filed: Aug. 5, 2022

Related U.S. Application Data
(63) Continuation of application No. PCT/US2021/016953, filed on Feb. 5, 2021.
(60) Provisional application No. 63/138,284, filed on Jan. 15, 2021.
(60) Provisional application No. 63/113,779, filed on Nov. 13, 2020.
(60) Provisional application No. 63/040,473, filed on Jun. 17, 2020.
(60) Provisional application No. 62/985,814, filed on Mar. 5, 2020.
(60) Provisional application No. 62/971,206, filed on Feb. 6, 2020.

(51) Int. Cl. C12Q 1/70; C12Q 1/6853; B01L 3/00; B01L 7/00

(52) U.S. Cl. CPC C12Q 1/701; C12Q 1/6853; B01L 3/502715; B01L 7/52; C12Q 2600/16; C12Q 2600/156; B01L 2300/0636; B01L 2300/087; B01L 2200/16; B01L 2300/0883

(57) ABSTRACT

Described herein, in certain embodiments, are methods of assaying for a segment of a coronavirus target nucleic acid in a sample. In some embodiments, the target nucleic acid is from a gene of SARS-CoV-2, or a variant thereof. In some embodiments, methods described herein comprise a) contacting the sample to: i) a detector nucleic acid; and ii) a composition comprising a programmable nuclease and a non-naturally occurring guide nucleic acid, and b) assaying for a change in a signal, wherein the change in the signal is produced by cleavage of the detector nucleic acid by the programmable nuclease. Also provided herein are devices for performing the methods of assaying for a segment of a coronavirus target nucleic acid described herein.

### CROSS REFERENCE

The present application claims priority to and benefit from U.S. Provisional Application No. 62/971,206, filed on Feb. 6, 2020, U.S. Provisional Application No. 62/985,814, filed on Mar. 5, 2020, U.S. Provisional Application No. 63/040,473, filed on Jun. 17, 2020, U.S. Provisional Application No. 63/113,779, filed on Nov. 13, 2020, and U.S. Provisional Application No. 63/138,284, filed on Jan. 15, 2021, the entire contents of each of which are herein incorporated by reference.

### SEQUENCE LISTING

The instant application contains a Sequence Listing which has been submitted electronically in XML file format and is hereby incorporated by reference in its entirety. Said XML copy, created on Oct. 10, 2022, is named 203477-730301_US_SL.xml and is 676,915 bytes in size.

### BACKGROUND

There have been recurrent large-scale epidemics from novel emerging viruses, including human immunodeficiency virus (HIV), SARS and MERS coronaviruses, influenza H1N1 virus, Ebola virus (EBOV), Zika virus (ZIKV), and SARS-CoV-2. The coronavirus can easily spread from an individual or environment to an individual. Person-to-person transmission of coronaviruses from infected individuals with subclinical or mild symptoms has been reported. Lack of rapid, accessible, and accurate molecular diagnostic testing has hindered the public health response to emerging viral threats, including coronaviruses. The detection of coronaviruses, especially at the early stages of infection, may provide guidance on treatment or intervention to reduce the progression or transmission of the ailment. Increased sensitivity of disease detection assays may provide earlier detection, leading to reduced transmission.

### SUMMARY

In various aspects, the present disclosure provides a method of assaying for a segment of a coronavirus target nucleic acid in a sample, the method comprising: a) contacting the sample to: i) a detector nucleic acid; and ii) a composition comprising a programmable nuclease and a non-naturally occurring guide nucleic acid that hybridizes to a segment of the target nucleic acid, wherein the programmable nuclease the detector nucleic acid upon hybridization of the non-naturally occurring guide nucleic acid to the segment of the coronavirus target nucleic acid; and b) assaying for a change in a signal, wherein the change in the signal is produced by cleavage of the detector nucleic acid.

In some aspects, the coronavirus target nucleic acid is from SARS-CoV-2. In some aspects, the target nucleic acid is from a variant of SARS-CoV-2, e.g., the United Kingdom (UK) or South African variants described herein. In some aspects, the coronavirus target nucleic acid is from an E gene, an N gene, or a combination thereof. In some aspects, the coronavirus target nucleic acid is from an E gene, an N gene, an S (Spike) gene or a combination thereof. In some aspects, the coronavirus target nucleic acid has a sequence of any one of SEQ ID NO: 179-SEQ ID NO: 184. In some aspects, the coronavirus target nucleic acid is from an S gene and comprises any of the mutations described in Table 11 herein. In some aspects, the guide nucleic acid is a guide RNA. In some aspects, the guide nucleic acid has at least 80%, at least 85%, at least 90%, at least 92%, at least 95%, at least 97%, or at least 99% sequence identify to any one of SEQ ID NO: 171-SEQ ID NO: 178, SEQ ID NO: 218, or SEQ ID NO: 219. In some aspects, the guide nucleic acid is selected from any one of SEQ ID NO: 171-SEQ ID NO: 178, SEQ ID NO: 218, or SEQ ID NO: 219. In some aspects, the guide nucleic acid comprises is selected from any of those listed in Table 15 herein. In some aspects, the guide nucleic acid has at least 80%, at least 85%, at least 90%, at least 92%, at least 95%, at least 97%, or at least 99% sequence identify to any one the sequences listed in Table 15 herein.

In some aspects, the method further comprising amplifying the coronavirus target nucleic acid. In some aspects, the amplifying the coronavirus target nucleic acid comprises contacting the sample to reagents for amplification. In some aspects, the contacting the sample to reagents for the amplification occurs prior to the contacting the sample to the detector nucleic acid to the detector nucleic acid and the composition. In some aspects, the contacting the sample to reagents for the amplification occurs concurrent to the contacting the sample to the detector nucleic acid to the detector nucleic acid and the composition. In some aspects, the amplifying comprises thermal cycling amplification. In some aspects, the amplifying comprises isothermal amplification.

In some aspects, the amplifying comprises transcription mediated amplification (TMA), helicase dependent amplification (HDA), circular helicase dependent amplification (cHDA), strand displacement amplification (SDA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), improved multiple displacement amplification (IMDA), or reverse-transcriptase polymerase chain reaction. In some aspects, the amplifying comprises loop mediated amplification (LAMP).

In some aspects, the reagents for amplification comprise an amplification primer, a polymerase, and dNTPs. In some aspects, the reagents for amplification comprise a FIP primer, a BIP primer, a LF primer, and a LB primer. In some aspects, the amplification primers are selected from SEQ ID NO: 194-SEQ ID NO: 199 or SEQ ID NO: 202-SEQ ID NO: 205. In some aspects, the primers are selected from those listed in Table 14 herein. In some aspects, the method further comprises reverse transcribing the coronavirus target nucleic acid. In some aspects, the reverse transcribing comprises contacting the sample to reagents for reverse transcription. In some aspects, the reagents for reverse transcription comprise a reverse transcriptase, an oligonucleotide primer, and dNTPs In some aspects, the contacting the sample to reagents for reverse transcription occurs prior to the contacting the sample to the detector nucleic acid to the detector nucleic acid and the composition, prior to the contacting the sample to the reagents for amplification, or prior to both.

In some aspects, the contacting the sample to reagents for reverse transcription occurs concurrent to the contacting the sample to the detector nucleic acid to the detector nucleic acid and the composition, concurrent to the contacting the sample to the reagents for amplification, or concurrent to both. In some aspects, the method further comprising assaying for a control sequence by contacting the control nucleic acid to a second detector nucleic acid and a composition comprising the programmable nuclease and a non-naturally occurring guide nucleic acid that hybridizes to a segment of the control nucleic acid, wherein the programmable nuclease the detector nucleic acid upon hybridization of the non-naturally occurring guide nucleic acid to the segment of the control nucleic acid.

In some aspects, the control nucleic acid is RNase P. In some aspects, the control nucleic acid has a sequence of SEQ ID NO: 220. In some aspects, the guide nucleic acid has at least 80%, at least 85%, at least 90%, at least 92%, at least 95%, at least 97%, or at least 99% sequence identify to SEQ ID NO: 178, SEQ ID NO: 218, or SEQ ID NO: 219. In some aspects, the guide nucleic acid is SEQ ID NO: 178, SEQ ID NO: 218, or SEQ ID NO: 219.

In some aspects, the method is carried out on a lateral flow strip. In some aspects, the lateral flow strip comprises a sample pad region, a control line, and a test line. In some aspects, the method further comprises adding the sample to the sample pad region. In some aspects, the presence or absence of an uncleaved reporter molecule is detected at the control line and the presence or absence of a cleaved reporter molecule is present at a test line. In some aspects, the method is carried out in a microfluidic cartridge. In some aspects, the method further comprises lysing the sample. In some aspects, the lysing the sample comprises contacting the sample to a lysis buffer.

In some aspects, the programmable nuclease comprises an RuvC catalytic domain. In some aspects, the programmable nuclease is a type V CRISPR/Cas effector protein. In some aspects, the type V CRISPR/Cas effector protein is a Cas12 protein. In some aspects, the Cas12 protein comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide. In some aspects, the Cas12 protein has at least 80%, at least 85%, at least 90%, at least 92%, at least 95%, at least 97%, or at least 99% sequence identity to any one of SEQ ID NO: 18-SEQ ID NO: 60. In some aspects, the Cas12 protein is selected from SEQ ID NO: 18-SEQ ID NO: 60.

In some aspects, the type V CRIPSR/Cas effector protein is a Cas14 protein. In some aspects, the Cas14 protein comprises a Cas14a polypeptide, a Cas14b polypeptide, a Cas14c polypeptide, a Cas14d polypeptide, a Cas14e polypeptide, a Cas14f polypeptide, a Cas14g polypeptide, a Cas14h polypeptide, a Cas14i polypeptide, a Cas14j polypeptide, or a Cas14k polypeptide. In some aspects, the Cas14 protein has at least 80%, at least 85%, at least 90%, at least 92%, at least 95%, at least 97%, or at least 99% sequence identity to any one of SEQ ID NO: 61-SEQ ID NO: 152.

In some aspects, the Cas14 protein is selected from SEQ ID NO: 61-SEQ ID NO: 152. In some aspects, the type V CRIPSR/Cas effector protein is a CasΦ protein. In some aspects, the CasΦ protein has at least 80%, at least 85%, at least 90%, at least 92%, at least 95%, at least 97%, or at least 99% sequence identity to any one of SEQ ID NO: 221-SEQ ID NO: 268. In some aspects, the CasΦ protein is selected from SEQ ID NO: 221-SEQ ID NO: 268. In some aspects, the method further comprises in vitro transcribing amplified coronavirus target nucleic acid. In some aspects, the in vitro transcribing comprises contacting the amplified coronavirus target nucleic acid to reagents for in vitro transcription.

In some aspects, the reagents for in vitro transcription comprise an RNA polymerase, a primer, and NTPs. In some aspects, the programmable nuclease comprises a HEPN cleaving domain. In some aspects, the programmable nuclease is a type VI CRISPR/Cas effector protein. In some aspects, the type VI CRISPR/Cas effector protein is a Cas13 protein. In some aspects, the Cas13 protein comprises a Cas13a polypeptide, a Cas13b polypeptide, a Cas13c polypeptide, a Cas13c polypeptide, a Cas13d polypeptide, or a Cas13e polypeptide. In some aspects, the Cas13 protein has at least 80%, at least 85%, at least 90%, at least 92%, at least 95%, at least 97%, or at least 99% sequence identity to any one of SEQ ID NO: 153-SEQ ID NO: 170. In some aspects, the Cas13 protein is selected from SEQ ID NO: 153-SEQ ID NO: 170.

In some aspects, the method further comprises multiplexed detection of more than one coronavirus target nucleic acid. In some aspects, the method further comprises multiplexed detection of more than one coronavirus target nucleic acid and a control nucleic acid. In some aspects, the multiplexed detection is carried out in a test tube, a well plate, a lateral flow strip, or a microfluidic cartridge. In some aspects, sample lysis, reverse transcription, amplification, in vitro transcription, detection, or any combination thereof is carried out in a single volume. In some aspects, sample lysis, reverse transcription, amplification, in vitro transcription, detection, or any combination thereof is carried out in separate volumes.

In various aspects, the present disclosure provides a composition comprising a non-naturally occurring guide nucleic acid having at least 80%, at least 85%, at least 90%, at least 92%, at least 95%, at least 97%, or at least 99% sequence identify to any one of SEQ ID NO: 171-SEQ ID NO: 177.

In some aspects, the guide nucleic acid is selected from any one of SEQ ID NO: 171-SEQ ID NO: 177. In some aspects, the composition further comprising any of the detector nucleic acids disclosed herein. In some aspects, the composition further comprising any of the programmable nucleases disclosed herein. In some aspects, the composition further comprising any of the reagents for amplification disclosed herein. In some aspects, the composition further comprising any of the reagents for reverse transcription disclosed herein. In some aspects, the composition further comprising any of the reagents for in vitro transcription disclosed herein. In some aspects, the composition further comprising any of the lysis buffers disclosed herein. In some aspects, the composition further comprising any of the control nucleic acids disclosed herein. In some aspects, the composition further comprising any of the guide nucleic acids disclosed herein. In some aspects, wherein the composition is present in any of the lateral flow strips disclosed herein. In some aspects, wherein the composition is present in any of the microfluidic cartridges disclosed herein.

Also described herein, in some aspects, is a device that comprises: a sample interface configured to receive a sample that comprises a coronavirus sequence of interest; a channel in fluid communication with the sample interface and a detection chamber, said channel comprising one or more movable mechanisms to separate the sample into a plurality of droplets, wherein said detection chamber is configured to receive and contact the plurality of droplets with at least one programmable nuclease probe disposed on a surface of said detection chamber, wherein said at least one programmable nuclease probe comprises a guide nucleic acid complexed with a programmable nuclease; and a plurality of sensors that determine a presence of said coronavirus sequence of interest by detecting a signal produced upon cleavage of a target nucleic acid region of said at least one sequence of interest by said at least one programmable nuclease probe.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates schematically the steps of preparing and detecting the presence or absence of SARS-CoV-2 (“2019-nCoV”) in a sample using reverse transcription and loop-mediated isothermal amplification (RT-LAMP) and Cas12 DNA Endonuclease-Targeted CRISPR Trans Reporter (DETECTR) reactions.

FIG. 2 shows the DETECTR assay results of the SARS-CoV-2 N-gene amplified with different primer sets (“2019-nCoV-set1” through “2019-nCoV-set12”) and detected using LbCas12a and a gRNA directed to the N-gene of SARS-CoV-2. A lower time to result is indicative of a positive result. For all primer sets, the time to result was lower for samples with more of the target sequence, indicating that the assay was sensitive for the target sequence.

FIG. 3 shows the individual traces of the DETECTR reactions plotted in FIG. 2 for the 0 fM and 5 fM samples. In each plot, the 0 fM trace is not visible above the baseline, indicating that there little to no non-specific detection.

FIG. 4 shows the time to result of a DETECTR reaction on samples containing either the N-gene, the E-gene, or no target (“NTC”) and amplified using primer sets directed to the E-gene of SARS-CoV-2 (“2019-nCoV-E-set13” through “2019-nCoV-E-set20”) or to the N-gene of SARS-CoV-2 (“2019-nCoV-N-set2l” through “2019-nCoV-N-set24”). The best performing primer set for specific detection of the SARS-CoV-2 E-gene was SARS-CoV-2-E-set14.

FIG. 5 shows the DETECTR assay results of the SARS-CoV-2 N-gene amplified with primer set 1 (“2019-nCoV-set1”) and detected using LbCas12a and either a gRNA directed to the N-gene of SARS-CoV-2 (“R1763-CDC-N2-Wuhan”) or a gRNA directed to the N-gene of SARS-CoV (“R1766-CDC-N2-SARS”).

FIG. 6 shows the results of a DETECTR reaction to determine the limit of detection of SARS-CoV-2 in a DETECTR reaction amplified using a primer set directed to the N-gene of SARS-CoV-2 (“2019-nCoV-N-set1”). Samples containing either 15,000, 4,000, 1,000, 500, 200, 100, 50, 20, or 0 copies of a SARS-CoV-2 N-gene target nucleic acid were detected. A gel of the N-gene RNA is shown below.

FIG. 7 shows the amplification of RNase P (GGAGTATTGAATAGTTGGGAATTGGAACCCCTCCAGGGGGAACCAAACATTGTC GTTCAGAAGAAGACAAAGAGAGATTGAAATGAAGCTGTTGATTTCAACACACAA ATTCTGGTGGTAGATGAAAGCAAAGCAAGTAAGTTTCTCCGAATCCCTAGTCAA CTGGAGGTAGAGACGGACTGCGCAGGTTAACTACAGCTCCCAGCATGCCTGAGG GGCGGGCTCAGCGGCTGCGCAGACTGGCGCGCGCGGACGGTCATGGGACTTCAG CATGGCGGTGTTTGCAGATTTGGACCTGCGAGCGGGTTCTGACCTGAAGGCTCTG CGCGGACTTGTGGAGACAGCCGCTCACCTTGGCTATTCAGTTGTTGCTATCAATC ATATCGTTGACTTTAAGGAAAAGAAACAGGAAATTGAAAAACCAGTAGCTGTTT CTGAACTCTTCACAACTTTGCCAATTGTACAGGGAAAATCAAGACCAATTAAAA TTTTAACTAGATTAACAATTATTGTCTCGGATCCATCTCACTGCAATGTTTTGAGA GCAACTTCTTCAAGGGCCCGGCTCTATGATGTTGTTGCAGTTTTTCCAAAGACAG AAAAGCTTTTTCATATTGCTTGCACACATTTAGATGTGGATTTAGTCTGCATAAC TGTAACAGAGAAACTACCATTTTACTTCAAAAGACCTCCTATTAATGTGGCGATT GACCGAGGCCTGGCTTTTGAACTTGTCTATAGCCCTGCTATCAAAGACTCCACAA TGAGAAGGTATACAATTTCCAGTGCCCTCAATTTGATGCAAATCTGCAAAGGAA AGAATGTAATTATATCTAGTGCTGCAGAAAGGCCTTTAGAAATAAGAGGGCCAT ATGACGTGGCAAATCTAGGCTTGCTGTTTGGGCTCTCTGAAAGTGACGCCAAGG CTGCGGTGTCCACCAACTGCCGAGCAGCGCTTCTCCATGGAGAAACTAGAAAAA CTGCTTTTGGAATTATCTCTACAGTGAAGAAACCTCGGCCATCAGAAGGAGATG AAGATTGTCTTCCAGCTTCCAAGAAAGCCAAGTGTGAGGGCTGAAAAGAATGCC CCAGTCTCTGTCAGCACTCCCTTCTTCCCTTTTATAGTTCATCAGCCACAACAAAA ATAAAACCTTTGTGTGATTTACTGTTTTCATTTGGAGCTAGAAATCAATAGTCTA TAAAAACAGTTTTACTTGCAATCCATTAAAACAACAAACGAAACCTAGTGAAGC ATCTTTTTAAAAGGCTGCCAGCTTAATGAATTTAGATGTACTTTAAGAGAGAAAG ACTGGTTATTTCTCCTTTGTGTAAGTGATAAACAACAGCAAATATACTTGAATAA AATGTTTCAGGTATTTTTGTTTCATTTTGTTTTTGAGATAGGGTCTTTGTTGCTCA GGCTGGAGTACAGTGGCATAATCACAGCTCACTGCAACCTCAATCCTGGGCTCA AGTGATCCTCCCGCTTCAGCCTCTCAAGCAGCGGGAACTACAGGTGTGCACTACC ACACCTGGCTATTTTTTTTTTTTTTTTTTTTTTCCCTTGTAGAGACATGGTCTCACT ATGTTGCTGAGGCTGGTCTCAAACTCCTAGGATCAAGCCATCCTCCCGCTTTGGC CTCCTAAAGTGCTGGGATTACATGAGCCACCACATGCAGCCAGATGTTTGAATAT TTTAAGAGCTTCTTTCGAAAGTTTCTTGTTCATACTCAAATAGTAGTTATTTTGAA GATATTCAAACTTATATTGAAGAAGTGACTTTAGTTCCTCTTGTTTTAAGCTTCTT TCATGTATTCAAATCAGCATTTTTTTCTAAGAAATTGCTATAGAATTTGTGGAAG GAGAGAGGATACACATGTAAAATTACATCTGGTCTCTTCCTTCACTGCTTCATGC CTACGTAAGGTCTTTGAAATAGGATTCCTTACTTTTAGTTAGAAACCCCTAAAAC GCTAATATTGATTTTCCTGATAGCTGTATTAAAAATAGCAAAGCATCGGACTGA, SEQ ID NO: 220) using a POP7 sample primer set. Samples were amplified using LAMP. DETECTR reactions were performed using a gRNA directed to RNase P (“R779”) and a Cas12 variant (SEQ ID NO: 28). Samples contained either HeLa total RNA or HeLa genomic DNA.

FIG. 8 shows the time to result of a multiplexed DETECTR reaction. Samples contained either in vitro transcribed N-gene of SARS-CoV-2 (“N-gene IVT”), in vitro transcribed E-gene of SARS-CoV-2 (“E-gene IVT”), HeLa total RNA, or no target (“NTC”). Samples were amplified using one or more primer sets directed to the SARS-CoV-2 N-gene (“set1”), the SARS-CoV-2 E-gene (“set14”), or RNase” (“RNaseP”).

FIG. 9 shows the time to results of a multiplexed DETECTR reaction with different combinations of primer sets directed to either SARS-CoV-2 N-gene (“set1”), SARS-CoV-2 E-gene (“set14”), or RNase P (“RNaseP”). Samples containing in vitro transcribed N-gene of SARS-CoV-2 (left, “N-gene IVT”) or in vitro transcribed E-gene of SARS-CoV-2 (right, “E-gene IVT”) were tested.

FIG. 10 shows the time to result of a multiplexed DETECTR reaction with the best performing primer set combinations from FIG. 8 and FIG. 9.

FIG. 11 schematically illustrates the sequence of the CDC-N2 target site used for detecting the N-2 gene of SARS-CoV-2. FIG. 11 discloses SEQ ID NOS 528-531, respectively, in order of appearance.

FIG. 12 schematically illustrates the sequence of a region of the SARS-CoV-2 N-gene (“N-Sarbeco”) target site. FIG. 12 discloses SEQ ID NOS 532-533 and 533-534, respectively, in order of appearance.

FIG. 13 shows the results of a DETECTR assay to determine the sensitivity of gRNAs directed to either N-gene of SARS-CoV-2 (“R1763”), the N-gene of SARS-CoV (“R1766”), or the N-gene of a Sarbeco coronavirus (“R1767”) for samples containing either the N-gene of SARS-CoV-2(“N-2019-nCoV”), the N-gene of SARS-CoV (“N-SARS-CoV”), or the N-gene of bat-SL-CoV45 (“N-bat-SL-CoV45”).

FIG. 14 schematically illustrates the sequence of a region of the SARS-CoV-2 E-gene (“E-Sarbeco”) target site. FIG. 14 discloses SEQ ID NOS 535-536 and 536-537, respectively, in order of appearance.

FIG. 15 shows the results of a DETECTR assay to determine the sensitivity of two gRNAs directed to a coronavirus N-gene for samples containing either the E-gene of SARS-CoV-2 (“E-2019-nCoV”), the E-gene of SARS-CoV (“E-SARS-CoV”), the E-gene of bat-SL-CoV45 (“E-bat-SL-CoV45”), or the E-gene of bat-SL-CoV21 (“E-bat-SL-CoV21”).

FIG. 16 shows the results of a lateral flow DETECTR reaction to detect the presence or absence of a SARS-CoV-2 N-gene target RNA using a Cas12 variant (SEQ ID NO: 28). Lateral flow test strips are shown. Samples either containing (“+”) or lacking (“−”) in vitro transcribed SARS-CoV-2 N-gene RNA (“N-gene IVT”) were tested. The top set of horizontal lines (denoted “test”) indicated the results of the DETECTR reaction.

FIG. 17 illustrates schematically the detection of a target nucleic acid using a programmable nuclease. Briefly, a Cas protein with trans collateral cleavage activity is activated upon binding to a guide nucleic acid and a target sequence reverse complementary to a region of the guide nucleic acid. The activated programmable nuclease cleaves a reporter nucleic acid, thereby producing a detectable signal.

FIG. 18 illustrates schematically detection of the presence or absence of a target nucleic acid in a sample. Select nucleic acids in a sample are amplified using isothermal amplification. The amplified sample is contacted to a programmable nuclease, a guide nucleic acid, and a reporter nucleic acid, as illustrated in FIG. 17. If the sample contains the target nucleic acid, a detectable signal is produced.

FIG. 19 shows the results of a DETECTR lateral flow reaction to detect the presence or absence of SARS-CoV-2 (“2019-nCoV”) RNA in a sample. Detection of RNase P is used as a sample quality control. Samples were in vitro transcribed and amplified (left) and detected using a Cas12 programmable nuclease (right). Samples containing (“+”) or lacking (“−”) in vitro transcribed SARS-CoV-2 RNA (“2019-nCoV IVT”) were assayed with a Cas12 programmable nuclease and gRNA directed to SARS-CoV-2 for either 0 min or 5 min. The reaction was sensitive for samples containing SARS-CoV-2.

FIG. 20 shows the results of a DETECTR reaction using an LbCas12a programmable nuclease (SEQ ID NO: 18) to determine the presence or absence of SARS-CoV-2 in patient samples.

FIG. 21 shows the results of a lateral flow DETECTR reaction to detect the presence or absence of SARS-CoV-2 in patient samples. Samples were detected with either a gRNA directed to SARS-CoV-2 or a gRNA directed to RNase P.

FIG. 22 shows technical specifications and assay conditions for detection of coronavirus using reverse transcription and loop-mediated isothermal amplification (RT-LAMP) and Cas12 detection.

FIG. 23 shows the results of a DETECTR assay evaluating multiple gRNAs for detecting SARS-CoV-2 using LbCas12a. Target nucleic acid sequences were amplified using primer sets to amplify the SARS-CoV-2 E-gene (“2019-nCoV-E-set13” through “2019-nCoV-E-set20” or the SARS-CoV-2 N-gene (“2019-nCoV-N-set2l” through “2019-nCoV-N-set24”).

FIG. 24 shows the results of a DETECTR assay evaluating multiple gRNAs for their utility in distinguishing between three different strains of coronavirus, SARS-CoV-2 (“COVID-2019”), SARS-CoV, or bat-SL-CoV45. Samples containing N-gene amplicons of either SARS-CoV-2 (“N-2019-nCoV”), SARS-CoV (“N-SARS-CoV”), or bat-SL-CoV45 (“N-bat-SL-CoV45”) were tested.

FIG. 25 shows the results of a DETECTR assay evaluating multiple gRNAs for their utility in distinguishing between three different strains of coronavirus, SARS-CoV-2 (“COVID-2019”), SARS-CoV, or bat-SL-CoV45. Samples containing E-gene amplicons of either SARS-CoV-2 (“N-2019-nCoV”), SARS-CoV (“N-SARS-CoV”), or bat-SL-CoV45 (“N-bat-SL-CoV45”) were tested.

FIG. 26 shows the results of a DETECTR assay evaluating LAMP primer sets for their utility in multiplexed amplification of SARS-CoV-2 targets. Samples were amplified with one or more primer sets directed to the SARS-CoV-2 N-gene (“set1”) or the SARS-CoV-2 E-gene (“set14”), or RNase P (“RNaseP”).

FIG. 27 shows the results of a DETECTR assay evaluating the sensitivity of an RT-LAMP amplification reaction to common sample buffers. Reactions were measured in universal transport medium (UTM, top) or DNA/RNA Shield buffer (bottom) at different buffer dilutions (from left to right: 1×, 0.5×, 0.25×, 0.125×, or no buffer).

FIG. 28 shows the results of a DETECTR assay to determine the limit of detection (LoD) of the DETECTR assay for SARS-CoV-2 (the virus attributed to the COVID-19 infection).

FIG. 29 shows the results of a DETECTR assay evaluating the target specificity of a gRNA directed to the N-gene of SARS-CoV-2 (“R1763-N-gene”) in a 2-plex multiplexed RT-LAMP reaction using an LbCas12a programmable nuclease (SEQ ID NO: 18).

FIG. 30 shows the results of a DETECTR assay evaluating the target specificity of a gRNA directed to the N-gene of SARS-CoV-2 (“R1763-N-gene”) or the E-gene of SARS-CoV-2 (“R1765-E-gene”) in a 3-plex multiplexed RT-LAMP reaction using an LbCas12a programmable nuclease (SEQ ID NO: 18).

FIG. 31 illustrates the design of detector nucleic acids compatible with a PCRD lateral flow device. Exemplary compatible detector nucleic acids, rep072, rep076, and rep100, are provided (left). These detector nucleic acids may be used in a PCRD lateral flow device (right) to detect the presence or absence of a target nucleic acid. The top right schematic illustrates an exemplary band configuration produced when contacted to a sample that does not contain a target nucleic acid. The bottom right schematic shows an exemplary band configuration produced when contacted to a sample that does contain a target nucleic acid. FIG. 31 discloses SEQ ID NOS 9, 185 and 185, respectively, in order of appearance.

FIG. 32A illustrates a genome map indicating the locations of the E (envelope) gene and the N (nucleoprotein) gene regions within a coronavirus genome. Corresponding regions or annealing regions of primers and probes relative to the E and N gene regions are shown below the respective gene regions. RT-LAMP primers are indicated by black rectangles, the binding position of the F1c and B1c half of the FIP primer (grey) is represented by a striped rectangle with dashed borders. Regions amplified in tests utilized by the World Health Organization (WHO) and the Center for Disease Control (CDC) are denoted as “WHO E amplicon” and “CDC N2 amplicon,” respectively.

FIG. 32B shows the results of a DETECTR assay evaluating the specificity or broad detection utility of gRNAs directed to the N-gene or E-gene of various coronavirus strains (SARS-CoV-2, SARS-CoV, or bat-SL-CoVZC45) using an LbCas12a programmable nuclease (SEQ ID NO: 18). The N gene gRNA used in the assay (left, “N-gene”) was specific for SARS-CoV-2, whereas the E gene gRNA was able to detect 3 SARS-like coronavirus (right, “E-gene”). A separate N gene gRNA targeting SARS-CoV and a bat coronavirus failed to detect SARS-CoV-2 (middle, “N-gene related species variant”).

FIG. 32C shows exemplary laboratory equipment utilized in the coronavirus DETECTR assays. In addition to appropriate biosafety protective equipment, the equipment utilized includes a sample collection device, microcentrifuge tubes, heat blocks set to 37° C. and 62° C., pipettes and tips, and lateral flow strips.

FIG. 32D illustrates an exemplary workflow of a DETECTR assay for the detection of a coronavirus in a subject. Conventional RNA extraction or sample matrix can be used as an input to DETECTR (LAMP pre-amplification and Cas12-based detection for NE gene, EN gene and RNase P), which is visualized by a fluorescent reader or lateral flow strip.

FIG. 32E shows lateral flow test strips (left) indicating a positive test result for SARS-CoV-2 N-gene (left, top) and a negative test result for SARS-CoV-2 N-gene (left, bottom). The table (right) illustrates possible test indicators and associated results for a lateral flow strip-based coronavirus diagnostic assay that tests for the presences of absence of the RNase P (positive control), SARS-CoV-2 N-gene, and coronavirus E-gene.

FIG. 33A illustrates cleavage of a detector nucleic acid labeled with FAM and biotin by a Cas12 programmable nuclease in the presence of a target nucleic acid (top). Schematics of lateral flow test strips (bottom) illustrate markings indicative of either the presence (“positive”) or absence (“negative”) of the target nucleic acid in the tested sample. The intact FAM-biotinylated reporter molecule flows to the control capture line. Upon recognition of the matching target, the Cas-gRNA complex cleaves the reporter molecule, which flows to the target capture line.

FIG. 33B shows the results of a DETECTR assay using LbCas12a to determine the effect of reaction time for a sample containing either 0 fM SARS-CoV-2 RNA or 5 fM SARS-CoV-2 RNA. Fluorescence signal of LbCas12a detection assay on RT-LAMP amplicon for SARS-CoV-2 N-gene saturated within 10 minutes. RT-LAMP amplicon was generated from 2 μL of 5 fM or 0 fM SARS-CoV-2 N-gene IVT RNA by amplifying at 62° C. for 20 minutes.

FIG. 33C shows lateral flow test strips assaying samples corresponding to the samples assayed by DETECTR in FIG. 33B. Bands corresponding to control (C) or test (T) are shown for samples containing either 0 fM SARS-CoV-2 RNA (“−”) or 5 fM SARS-CoV-2 RNA (“+”) as a function of reaction time. LbCas12a on the same RT-LAMP amplicon produced visible signal through lateral flow assay within 5 minutes.

FIG. 33D shows the results of a DETECTR assay with LbCas12a (middle) or a CDC protocol (left) to determine the limit of detection of SARS-CoV-2. Signal is shown as a function of the number of copies of viral genome per reaction. Representative lateral flow results for the assay shown for 0 copies/μL and 10 copies/μL (right).

FIG. 33E shows patient sample DETECTR data. Clinical samples from 6 patients with COVID-19 infection (n=11, 5 replicates) and 12 patients infected with influenza or one of the 4 seasonal coronaviruses (HCoV-229E, HCoV-HKU1, HCoV-NL63, HCoV-OC43) (n=12) were analyzed using SARS-CoV-2 DETECTR (shaded boxes). Signal intensities from lateral flow strips were quantified using ImageJ and normalized to the highest value within the N gene, E gene or RNase P set, with a positive threshold at five standard deviations above background. Final determination of the SARS-CoV-2 test was based on the interpretation matrix in FIG. 32E. FluA denotes Influenza A, and FluB denotes Influenza B. HCoV denotes human coronavirus.

FIG. 33F shows lateral flow test strips testing for SARS-CoV-2 in a patient with COVID-19 (positive for SARS-CoV-2, “patient 1”), a no target control sample lacking the target nucleic acid (“NTC”), and a positive control sample containing the target nucleic acid (“PC”). All three samples were tested for the presence of the SARS-CoV-2 N-gene, the SARS-CoV-2 E-gene, and RNase P.

FIG. 33G shows performance characteristics of the SARS-CoV-2 DETECTR assay. 83 clinical samples (41 COVID-19 positive, 42 negative) were evaluated using the fluorescent version of the SARS-CoV-2 DETECTR assay. One sample (COVID19-3) was omitted due to failing assay quality control. Positive and negative calls were based on criteria described in FIG. 32E. fM denotes femtomolar; NTC denotes no-template control; PPA denotes positive predictive agreement; NPA denotes negative predictive agreement.

FIG. 34 shows a table comparing the SARS-CoV-2 DETECTR assay with RT-LAMP of the present disclosure to the SARS-CoV-2 assay with a quantitative reverse transcription polymerase chain reaction (qRT-PCR) detection method. The N-gene target in the DETECTR RT-LAMP assay is the same as the N-gene N2 amplicon detected in the qRT-PCR assay.

FIG. 35A shows the time to result of an RT-LAMP amplification under different buffer conditions. Time to results was calculated as the time at which the fluorescent value is one third of the max for the experiment. Reactions that failed to amplify are reported with a value of 20 minutes and labeled as “no amp.” Time to result was determined for different starting concentrations of target control plasmid in either water, 10% phosphate buffered saline (PBS), or 10% universal transport medium (UTM). A lower time to result indicates faster amplification.

FIG. 35B shows the results of an RT-LAMP assay to determine the amplification efficiency of the N-gene of SARS-CoV-2, the E-gene of SARS-CoV-2, and RNase P in either 5% UTM, 5% PBS, or water. Samples containing 0.5 fM N-gene in vitro transcribed, 0.5 fM of E-gene in vitro transcribed, and 0.8 ng/μL HeLa total RNA (“N+E+total RNA”) or no target controls (“NTC”) were tested.

FIG. 35C shows amplification of RNA directly from nasal swabs in PBS. Time to result was measured as a function of PBS concentration. Nasal swabs (“nasal swab”) were either spiked with HeLa total RNA (left, “total RNA: 0.08 ng/uL”) or water (right, “total RNA: 0 ng/uL”). Samples without a nasal swab (“no swab”) were compared as controls.

FIG. 36A shows raw fluorescence curves generated by LbCas12a (SEQ ID NO: 18) detection of SARS-CoV-2 N-gene (n=6). The curves showed saturation in less than 20 minutes.

FIG. 36B shows the limit of detection of a DETECTR assay for the SARS-CoV-2 N-gene detected with LbCas12a, as determined from the raw fluorescence traces shown in FIG. 36A. Fluorescence intensity was measured with decreasing concentration (copies per mL) of SARS-CoV-2 N-gene.

FIG. 36C shows the time to result of the limit of detection DETECTR assay, as determined from the raw fluorescence traces shown in FIG. 36A. A lower time to result indicates faster amplification and detection.

FIG. 37A shows the results of a DETECTR assay using LbCas12a to determine the effect of reaction time for a sample containing either 0 fM SARS-CoV-2 RNA or 5 fM SARS-CoV-2 RNA.

FIG. 37B shows lateral flow test strips assaying samples corresponding to the samples assayed by DETECTR in FIG. 37A. Bands corresponding to control (C) or test (T) are shown for samples containing either 0 fM SARS-CoV-2 RNA (“−”) or 5 fM SARS-CoV-2 RNA (“+”) as a function of reaction time.

FIG. 38 shows the results of a DETECTR assay to determine the cross-reactivity of gRNAs for different human coronavirus strains. Samples containing in vitro transcribed RNA of the SARS-CoV-2 N-gene, the SARS-CoV N-gene, the bat-SL-CoVZC45 N-gene, the SARS-CoV-2 E-gene, the SARS-CoV E-gene, or the bat-SL-CoVZC45 E-gene, or clinical samples positive for CoV-HKU1, CoV-299E, CoV-OC43, or CoV-NL63 were tested. HeLa total RNA was tested as a positive control for RNase P, and a sample lacking a target nucleic acid (“NTC”) was tested as a negative control.

FIG. 39A shows a sequence alignment of the target sites targeted by the N-gene gRNA for three coronavirus strains. The N gene gRNA #1 is compatible with the CDC-N2 amplicon, the N gene gRNA #2 is compatible with WHO N-Sarbeco amplicon. FIG. 39A discloses SEQ ID NOS 538-543, respectively, in order of appearance.

FIG. 39B shows a sequence alignment of the target sites targeted by the E-gene gRNA for three coronavirus strains. The two E gene gRNAs tested (E gene gRNA #1 and E gene gRNA #2) are compatible with the WHO E-Sarbeco amplicon. FIG. 39B discloses SEQ ID NOS 544-549, respectively, in order of appearance.

FIG. 40A-FIG. 40C show DETECTR kinetic curves on COVID-19 infected patient samples. Ten nasal swab samples from 5 patients (COVID19-1 to COVID19-10) were tested for SARS-CoV-2 using two different genes, N2 and E as well as a sample input control, RNase P. FIG. 40A shows using the standard amplification and detection conditions, 9 of the 10 patients resulted in robust fluorescence curves indicating presence of the SARS-CoV-2 E-gene (20 minute amplification, signal within 10 minutes). FIG. 40B shows the SARS-CoV-2 N-gene required extended amplification time to produce strong fluorescence curves (30 minute amplification, signal within 10 minutes) for 8 of the 10 patients. FIG. 40C shows that as a sample input control, RNase P was positive for 17 of the 22 total samples tested (20 minute amplification, signal within 10 minutes).

FIG. 41 shows DETECTR analysis of SARS-CoV-2 identifies down to 10 viral genomes in approximately 30 min (20 min amplification, 10 min DETECTR). Duplicate LAMP reactions were amplified for twenty min followed by LbCas12a DETECTR analysis.

FIG. 42 shows the raw fluorescence at 5 minutes for the LbCas12a DETECTR analysis provided in FIG. 41. The limit of detection of the SARS-CoV-2 N-gene was determined to be 10 viral genomes per reaction (n=6).

FIG. 43 shows lateral flow DETECTR results on 10 COVID-19 infected patient samples and 12 patient samples for other viral respiratory infections. Ten samples from 6 patients (COVID19-1 to COVID19-5) with one nasopharyngeal swab (A) and one oropharyngeal swab (B) were tested for SARS-CoV-2 using two different genes, N2 and E as well as a sample input control, RNase P. Results were analyzed in accordance with the guidance provided in FIG. 44.

FIG. 44 shows instructions for the interpretation of SARS-CoV-2 DETECTR lateral flow results.

FIG. 45A-C show fluorescent DETECTR kinetic curves performed on 11 COVID-19 infected patient samples and 12 patient samples for other viral respiratory infections. Ten nasopharyngeal/oropharyngeal swab samples from 6 patients (COVID19-1 to COVID19-6) were tested for SARS-CoV-2 using two different genes, N2 and E as well as a sample input control, RNase P.

FIG. 45A shows samples tested using the standard amplification and detection conditions, 10 of the 12 COVID-19 positive patient samples resulted in robust fluorescence curves indicating presence of the SARS-CoV-2 E gene (20-minute amplification, signal within 10 min). No E gene signal was detected in the 12 other viral respiratory clinical samples.

FIG. 45B shows samples tested for the presence of the SARS-CoV-2 N gene using an extended amplification time to produce strong fluorescence curves (30-minute amplification, signal within 10 min) for 10 of the 12 COVID-19 positive patient samples. No N gene signal was detected in the 12 other viral respiratory clinical samples.

FIG. 46A shows heatmaps of SARS-CoV-2 DETECTR assay results for clinical samples with the test interpretation indicated. Results of lateral flow SARS-CoV-2 DETECTR assay (top) quantified by ImageJ Gel Analyzer tools for SARS-CoV-2 DETECTR on 24 clinical samples (12 COVID-19 positive) show 98.6% ( 71/72 strips) agreement with the results of the fluorescent version of the assay (bottom). Both assays were run with 30-minute amplification, Cas12 reaction signal taken at 10 min. Presumptive positive indicated by (+) in orange (bottom, column 4).

FIG. 46B shows heatmaps of SARS-CoV-2 DETECTR assay results for clinical samples with the test interpretation indicated. The top plot shows result of fluorescent SARS-CoV-2 DETECTR assay on an additional 30 COVID-19 positive clinical samples (27 positive, 1 presumptive positive, 2 negative). Presumptive positive indicated by (+) in orange (top, column 9). The bottom plot shows result of fluorescent SARS-CoV-2 DETECTR assay on an additional 30 COVID-19 negative clinical samples (0 positive, 30 negative).

FIG. 47 shows the time to result for RT-LAMP amplification of RNase P POP7 with different primer sets. Time to result was determined for samples amplified with primer sets 1-10. Primer set 1 corresponds to SEQ ID NO: 206-SEQ ID NO: 211, and primer set 9 corresponds to SEQ ID NO: 212-SEQ ID NO: 217.

FIG. 48 shows raw fluorescence over time of a DETECTR reaction performed on RNase P POP7 amplified using RT-LAMP with primer set 1 or primer set 9 and detected with R779, R780, or R1965 gRNAs. The DETECTR reaction was carried out at 37° C. for 90 minutes. The amplicon generated by the set 1 primers were detected without background (dotted line) by R779.

FIG. 49A shows the time to result of RNase P POP7 detection in samples containing 10-fold dilutions of total RNA amplified using RT-LAMP with primer set 1 or primer set 9. Amplification was carried out at 60° C. for 30 minutes.

FIG. 49B shows a DETECTR reaction of the RNase P POP7 amplicons shown in FIG. 49A and detected using gRNA 779 (SEQ ID NO: 178) or gRNA 1965 (SEQ ID NO: 218). Samples amplified using primer set 1 were detected with gRNA 779 and samples amplified with primer set 9 were detected with gRNA 1965. The DETECTR reaction was carried out at 37° C. for 90 minutes.

FIG. 50 shows the results of amplification of a SeraCare target nucleic acid using LAMP under different lysis conditions. Samples were amplified in a low pH buffer containing either buffer (top plots) or a viral lysis buffer (“VLB,” bottom plots). Buffers contained no reducing agent (“Control,” columns 1 and 4), Reducing Agent B (columns 2 and 5), or Reducing Agent A (columns 3 and 6). Samples were incubated for 5 minutes at either room temperature (left plots) or 95° C. (right plots). Samples containing either no target (“NTC”), 2.5, 25, or 250 copies per reaction. Assays were performed in triplicate using 5 μL of sample in a 25 μL reaction.

FIG. 51 shows the results of amplification of a SeraCare standard target nucleic acid using LAMP under different lysis conditions. Samples were amplified in a low pH buffer containing either buffer (left plots) or a viral lysis buffer (“VLB,” right plots). Buffers contained no reducing agent (“Control”), Reducing Agent B, or Reducing Agent A. Samples were incubated for 5 minutes at either room temperature (top plots) or 95° C. (bottom plots). Samples containing either no target (“NTC”), 1.5, 2.5, 15, 25, 150, or 250 copies per reaction. Assays were performed in triplicate using 3 μL of sample in a 15 μL reaction or 5 μL of sample in a 25 μL reaction.

FIG. 52 shows amplification of a SARS-CoV-2 N gene (“N”) and an RNase P sample input control nucleic acid (“RP”) in the presence of six different viral lysis buffers (“VLB,” “VLB-D,” “VLB-T,” “Buffer,” “Buffer-A,” and “Buffer-B”). Buffer-A contains Buffer with Reducing Agent A and Buffer-B contains Buffer with Reducing Agent B. Shaded squares indicate rate of amplification, with darker shading indicating faster amplification. Amplification was performed at either 95° C. (“95 C”) or room temperature (“RT”) on high, medium, or low titer COVID-19 positive patient samples (“16.9,” “30.5,” and “33.6,” respectively). Samples were measured in duplicate.

FIG. 53A and FIG. 53B show photos of cartridges designed for use in a DETECTR assay.

FIG. 54A and FIG. 54B schematic view of the cartridge pictured in FIG. 53A.

FIG. 55A-FIG. 55D show schematics of cartridges designed for use in a DETECTR assay. FIG. 55A shows a cartridge with circular reagent storage wells and a z-direction high resistance serpentine path. FIG. 55B shows a cartridge with elongated reagent storage wells and a z-direction high resistance serpentine path. FIG. 55C shows a cartridge with circular reagent storage wells and an xy-direction high resistance serpentine path. FIG. 55D shows a cartridge with elongated reagent storage wells and an xy-direction high resistance serpentine path.

FIG. 56A-FIG. 56D show schematics of cartridges designed for use in a DETECTR assay. FIG. 56A shows a cartridge with serpentine resistance channels for sample metering which are serpentine on a different plane or layer than the sample metering channel. FIG. 56B shows a cartridge with serpentine resistance channels for sample metering which are serpentine on the same plane or layer than the sample metering channel. FIG. 56C shows a cartridge with right angle arduous path resistance paths for sample metering and a DETECTR sample metering inlet on a different plane or layer than the sample metering channel. FIG. 56D shows a cartridge with right angle arduous path resistance paths for sample metering and a DETECTR sample metering inlet on the same plane or layer than the sample metering channel.

FIG. 57A shows features of a cartridge designed for use in a DETECTR assay.

FIG. 57B shows a manufacturing scheme (left and middle) for manufacturing a cartridge of the present disclosure and a readout device (right) for detecting a sample in a cartridge.

FIG. 58A shows a schematic of a cartridge manifold for heating regions of a cartridge of the present disclosure. The cartridge manifold has an integrated heating zone with integrated air supply connections and integrated O-ring grooves for air supply interface. The cartridge manifold contains an insulation zone to thermally separate the amplification temperature zone from the detection temperature zone and to maintain the appropriate temperature of the amplification chambers and the detection chambers of the cartridge.

FIG. 58B shows two production methods for producing the cartridges described herein. In a first manufacturing method (left), a cartridge is manufactured using two-dimensional (2D) lamination of multiple layers. In a second manufacturing method (right), a part containing consolidated, complex features is injection molded and sealed by lamination.

FIG. 58C shows a schematic of a cartridge with a luer slip adapter for coupling the cartridge to a syringe. The adapter can form a tight fit seal with a slip luer tip. The adapter is configured to function with any of the cartridges disclosed herein.

FIG. 59A and FIG. 59B show schematics of an integrated flow cell for use with a microfluidic cartridge. The integrated flow cell contains three regions, a lysis region, an amplification region, and a detection region. The lysis region is long enough to accommodate a microfluidic chip shop sample lysis flow cell. The lysis flow cell may be combined with the amplification and detection chambers on the cartridges disclosed herein.

FIG. 60 shows details of the inlet channels on a cartridge of the present disclosure.

FIG. 61 shows a workflow for performing a DETECTR assay using a microfluidic cartridge of the present disclosure. The cartridge (“chip”) is loaded with a sample and reaction solutions. The amplification chamber (“LAMP chamber”) is heated to 60° C. and the sample is incubated in the amplification chamber for 30 minutes. The amplified sample (“LAMP amplicon”) is pumped to the DETECTR reaction chambers, and the DETECTR reagents are pumped to the DETECTR reaction chambers. The DETECTR reaction chambers are heated to 37° C. and the sample is incubated for 30 minutes. The fluorescence in the DETECTR reaction chambers is measured in real time to produce a quantitative result.

FIG. 62 shows a schematic of a system electronics architecture of a cartridge manifold compatible with the cartridges disclosed herein. The electronics are configured to heat a first zone of a cartridge to 37° C. and a second zone of the cartridge to 60° C.

FIG. 63A and FIG. 63B show schematics of a cartridge manifold for heating and detecting a cartridge of the present disclosure. The manifold is configured to accept a cartridge, facilitate a DETECTR reaction, and read the resulting fluorescence of the DETECTR reaction.

FIG. 64A shows an example of a fluorescent sample in a cartridge and illuminated with a cartridge manifold. The positive control well contains reagents and an amplified sample following a 30 minute amplification step at 60° C. and a 30 minute detection step at 37° C. The empty well serves as a pseudo negative sample.

FIG. 64B shows a detection manifold of the present disclosure.

FIG. 65 shows a cartridge manifold for heating and detecting a cartridge of the present disclosure.

FIG. 66A and FIG. 66B show detection of a fluorescence signal produced by a DETECTR reaction performed in a microfluidic cartridge facilitated by a detection manifold.

FIG. 67A, FIG. 67B, FIG. 68A, and FIG. 68B show thermal testing summaries for an amplification chamber heated to 60° C. (FIG. 67A and FIG. 68A) or a DETECTR chamber heated to 37° C. (FIG. 67B and FIG. 68B).

FIG. 69A shows the DETECTR results run on a plate reader at a gain of 100, using the LAMP product from the microfluidic cartridge as an input. The samples were run in duplicate with a single non-template control (NTC).

FIG. 69B shows three LAMP products run on a plate reader using samples from a microfluidic chip. The LAMP reactions are numbered in the order that the chips were run (LAMP_1 was run first, etc.). The donor was homozygous for SNP A, and in accordance with that crRNA 570 comes up first. The ATTO 488 was used as a fluorescence standard.

FIG. 70A shows an image of a loaded microfluidic chip.

FIG. 70B shows results of a DETECTR reaction measured on a plate reader after 30 minutes of LAMP amplification.

FIG. 71A, FIG. 71B, FIG. 71C, and FIG. 71D show results of the coronavirus DETECTR reaction. The two reaction chambers with 10 copies input to LAMP resulted in a rapidly increasing DETECTR signal. All NTCs were negative. With 10 copies input into LAMP, the DETECTR signal gradually increased over the course of the reaction, as shown in the photodiode measurements below in FIG. 71C. The negative controls in FIG. 71D indicated an absence of contamination.

FIG. 72A, FIG. 72B, FIG. 72C, and FIG. 72D show the results of the repeated coronavirus DETECTR reaction.

FIG. 73A, FIG. 73B, FIG. 74A, FIG. 74B, and FIG. 74C show the photodiode measurements for an influenza B DETECTR reaction in a microfluidic cartridge.

FIG. 75 provides a design for an injection molded-cartridge containing a sample input chamber and multiple chambers in which portions of the sample can be subjected to amplification and detector reactions.

FIG. 76 provides a design for a device comprising a detector diode array and heating panels that is capable of utilizing the injection-molded cartridge shown in FIG. 75.

FIG. 77 and FIG. 78 show fluorescence data from a series of DETECTR reactions performed on samples subjected to different dual-lysis amplification buffers.

FIG. 79 panel (a) provides a design for an injection-molded cartridge for performing multiple amplification and DETECTR reactions on a sample. Panel (b) provides a design for a device configured to utilize the injection-molded cartridge and measure fluorescence from the DETECTR reactions performed in the cartridge.

FIG. 80 provides a method for utilizing the injection-molded cartridge and device shown in FIG. 79 for performing parallel amplification and DETECTR reactions on a sample.

FIG. 81 shows diode arrays and dye-loaded reaction compartments from the injection-molded cartridge and device in FIG. 79.

FIG. 82 shows a possible design for an injection molded cartridge comprising one sample chamber connected to 5 amplification chamber, and 2 Detection chambers connected to each amplification chamber. Thus, the device is capable of performing 10 parallel DETECTR reactions on a single sample.

FIG. 83 shows a possible design for an injection molded cartridge comprising one sample chamber connected to 4 amplification chamber, and 2 Detection chambers connected to each amplification chamber. The injection-molded cartridge comprises a series of valves and pumps or ports to pump manifolds that control flow throughout the cartridge.

FIG. 84 shows a possible design for an injection molded cartridge comprising one sample chamber connected to 4 amplification chamber, 2 Detection chambers connected to each amplification chamber, and a reagent chamber connected to the sample chamber.

FIG. 85 provides a top-down view of an injected-molded cartridge design with the reagent chambers in the flow paths leading to the amplification and Detection chambers.

FIG. 86 shows a portion of an injected-molded cartridge design with a sample chamber capable of connecting to multiple reagent and amplification chambers by a single rotating valve.

FIG. 87 shows a portion of an injected-molded cartridge design with a sliding valve connecting multiple compartments. Panels A-C show different positions that the sliding valve is capable of adopting.

FIG. 88 panel A shows a possible design for an injection-molded cartridge with a casing. Panel B provides a physical model of the design shown in panel A.

FIG. 89 panel A provides a bottom-up view a design of an injection-molded cartridge with a casing. Panel B provides a view of the top of the injection-molded cartridge.

FIG. 90 provides multiple views of an injection-molded cartridge with a sliding valve.

FIG. 91 provides two views of a portion of an injection-molded cartridge with multiple reagent wells that lead to transparent reaction chambers.

FIG. 92 panels A-B provide top-down views of an injection-molded cartridge design. Panel C shows a picture of a physical model of the injection-molded cartridge.

FIG. 93 shows a picture of an injection-molded cartridge housed in a device containing a diode array.

FIG. 94 shows a graphic user interface for controlling a device that contains an injection-molded cartridge and a diode array for detection.

FIG. 95 shows results from a series of fluorescence experiments utilizing an 8-diode detector array, an 8 chamber injection-molded cartridge, and dyes.

FIG. 96 shows fluorescence results from a series of HERC2 targeting DETECTR reactions and buffer controls, measured with an 8-diode detector array.

FIG. 97 shows an injection molded cartridge inserted into a device, with 8 chambers containing DETECTR reactions.

FIG. 98 shows a panel of gRNAs that bind to Matrix Protein 1 RNA from Influenza A virus (IAV-MP gRNAs) and gRNAs that bind to Polymerase Basic Protein 2 RNA from Influenza A virus (IAV-PB2 gRNAs) evaluated for detection efficiency. Darker squares in the background subtracted row indicate greater efficiency of detecting IAV target nucleic acids.

FIG. 99 shows a graph of pools of gRNA versus background subtracted fluorescence in a DETECTR reaction for detection of 160 fM of target nucleic acids. The number of pooled gRNA increases from 1 to 10 different gRNAs along the x-axis. This graph shows increasing signal from 1 gRNA to 10 pooled gRNAs.

FIG. 100 shows an exemplary assay design for a PON 5-plex panel comprising pooled CRISPR-Cas complexes in discrete regions for viral detection. The discrete regions are for detection of: (1) SARS-CoV-2, (2) Flu A, (3) Flu B, (4) Pan-CoV, and (5) Endogenous human control. The (1) SARS-CoV-2 region comprises gRNA for detecting N-gene targets and E-gene targets, the (2) Flu A region comprises gRNA for detecting H1N1 targets, H3N2 targets, and H1N1 pdm2009 targets, the (3) Flu B region comprises gRNA for detecting Yamagata targets and Victoria targets, the (4) Pan-CoV region comprises gRNA for detecting HCoV-OC43 targets, HCoV-NL63 targets, HCoV-229E targets, and HCoV-HKU1 targets, and the (5) Endogenous human control region comprises gRNA for human rpp30 targets. Each region can comprise pooled gRNA. For example, the gRNAs for the Flu A region bind to target sites that are 98% conserved among H1N1, H3N2, and H1N1 pdm2009, such as Matrix Protein 1 (MP), Nonstructural Protein 1 (NS), Neuraminidase (NA), Nucleoprotein (NP), Hemagglutinin (HA), PB1, Polymerase Acidic Protein (PA), and Polymerase Basic Protein 2 (PB2). Detected signal from each region can indicate the detection of a target within that region.

FIG. 101 depicts the amino acid sequence of the SARS-COV-2 Spike glycoprotein described herein. FIG. 101 discloses SEQ ID NO: 550.

FIGS. 102 A and B depict the nucleotide sequence of the SARS-COV-2 S gene described herein. FIG. 102 discloses SEQ ID NO: 551.

FIG. 103 shows the assay results from the testing of buffer and polymerase combinations that are suitable for enabling the rapid amplification of SARS-CoV-2.

FIG. 104 shows the assay results from the further optimization of buffer and polymerase combinations that are suitable for enabling the rapid amplification of SARS-CoV-2.

FIG. 105 shows the limit of detection of the FASTR assay and results of detection at a single-copy of SARS-CoV-2.

FIG. 106 shows the results from the optimization of rapid cycling times including the denaturation and annealing/extension times in the FASTR assay.

FIG. 107 shows the results from the optimization of FASTR assay conditions to minimize the reverse-transcription time (RT time).

FIG. 108 shows the effect of buffer pH conditions on FASTR assay performance.

FIG. 109 shows the performance of FASTR assay when combined with various crude lysis buffers.

FIG. 110 shows the results from a multiplexed FASTR assay under non-optimized conditions.

FIG. 111 shows the results of optimization of multiplexed FASTR assay reaction conditions containing different combinations of buffers, primer concentrations, dNTPs, DMSO, and identification of robust assay conditions.

FIG. 112 shows the performance of the optimized multiplexed FASTR assay at different concentrations of human RNA and viral RNA.

FIG. 113 shows the results of a guide screen designed to screen for guide RNAs that can detect the E484K SNP location within the spike region of SARS-CoV-2 and that are further capable of distinguishing between mutant (E484K) and WT SARS-CoV-2. FIG. 113 discloses SEQ ID NOS 552-553, respectively, in order of appearance.

FIG. 114 shows the results of a guide screen designed to screen for guide RNAs that can detect the N501Y SNP location within the spike region of SARS-CoV-2 and that are further capable of distinguishing between mutant (N501Y) and WT SARS-CoV-2. FIG. 114 discloses SEQ ID NOS 554-555, respectively, in order of appearance.

### DETAILED DESCRIPTION

The present disclosure provides various compositions and methods of use thereof for assaying for and detecting coronavirus in a sample. In particular, the various methods, reagents, and devices disclosed herein use a programmable nuclease complexed with guide nucleic acid sequence to detect the presence or absence of, and/or quantify the amount of, nucleic acids from coronavirus. Disclosed herein is a CRISPR (clustered regularly interspaced short palindromic repeats)-Cas12 based assay for detection of coronaviruses, including SARS-CoV-2, in patient samples in approximately 30 minutes. The detection assays disclosed herein may provide low cost, portable, and accurate detection of coronaviruses and may be performed using commercially available reagents. Such an assays may be referred to herein as a coronavirus DNA Endonuclease-Targeted CRISPR Trans Reporter (DETECTR) assays. The coronavirus can be SARS-CoV-2 (also known as 2019 novel coronavirus, Wuhan coronavirus, or 2019-nCoV), 229E (alpha coronavirus), NL63 (alpha coronavirus), OC43 (beta coronavirus), HKU1 (beta coronavirus), MERS-CoV, or SARS-CoV. The coronavirus may be a variant of SARS-CoV-2, particularly the United Kingdom (UK) variant known as 20B/501Y.V1, VOC 202012/01, or B.1.1.7 lineage, or the South African variant known as: 20C/501Y.V2 or B.1.351 lineage. The genetic characteristics of these variants are discussed in Leung et. al, Early transmissibility assessment of the N501Y mutant strains of SARS-CoV-2 in the United Kingdom, October to November 2020, Euro Surveill. 2021; 26(1) and in Tegally et. al., Emergence and rapid spread of a new severe acute respiratory syndrome-related coronavirus 2 (SARS-CoV-2) lineage with multiple spike mutations in South Africa, MedRxiv 2020.12.21. In some embodiments, the compositions and methods disclosed herein specifically target and assay for the SARS-CoV-2 coronavirus. The compositions and methods disclosed herein may be used to detect the presence or absence of the SARS-CoV-2 in a patient sample. In some embodiments, a patient may be diagnosed with COVID-19 if the presence of SARS-CoV-2 is detected in a sample from the patient. The assays disclosed herein may provide single nucleotide target specificity, enabling specific detection of a single coronavirus. The terms “2019-nCoV,” “SARS-CoV-2,” and “COVID-19” may be used interchangeably herein. DETECTR assays disclosed herein may use reverse transcription (RT) and/or isothermal amplification (e.g., loop-mediated amplification (LAMP)) of RNA (e.g., RNA extracted from a patient sample), followed by Cas12 detection of predefined coronavirus sequences, followed by cleavage of a reporter molecule to detect the presence of a virus. A DETECTR assay may target the E (envelope) genes or N (nucleoprotein) genes of a coronavirus (e.g., SARS-CoV-2). In some cases, a DETECTR assay may target the S (spike) gene of a coronavirus (e.g., SARS-CoV-2) or coronavirus variant. Isothermal amplification may be performed to amplify one or more regions of an N-gene of a coronavirus, an E-gene of a coronavirus, or both. Also disclosed herein are primer sets designed for LAMP amplification of one or more regions of an N-gene of a coronavirus, an E-gene of a coronavirus, or both. Also disclosed herein are primer sets designed for reverse-transcriptase PCR amplification of one or more regions of an S-gene of a coronavirus. Any nucleic acid of the SARS-CoV-2 can be assayed for using the compositions and methods disclosed herein. In some embodiments, the target nucleic acid comprises the N gene or the E gene of coronavirus and can be assayed for using the compositions and methods disclosed herein. Disclosed herein are guide nucleic acids (gRNAs) for the specific detection of the N-gene of a coronavirus strain. Also disclosed herein are guide nucleic acids (gRNAs) for the specific detection of mutations comprised in the S-gene of one or more coronavirus strains. Disclosed herein are gRNAs for the broad detection of the E-gene of one or more coronavirus strains.

In some embodiments, a programmable nuclease can be used for detection of a target nucleic acid from coronavirus (e.g., from a coronavirus such as SARS-CoV-2) in a sample from a subject. For example, a programmable nuclease can be complexed with a guide nucleic acid that hybridizes to a target sequence of a target nucleic acid from coronavirus. The complex can be contacted to a sample from a subject. The subject may or may not be infected with coronavirus. The target nucleic acid in the sample can be reverse transcribed (RT) and amplified by thermal amplification (e.g., PCR) or isothermal amplification (e.g., LAMP). In some embodiments, reverse transcription and isothermal amplification may be performed simultaneously. If the subject is infected with coronavirus, the guide nucleic acid hybridizes to the target nucleic acid leading to activation of programmable nuclease. Upon activation, the programmable nuclease can cleave a detector nucleic acid, wherein the detector nucleic acid comprises a detectable label attached to a polynucleotide (e.g., polydeoxyribonucleotide or polyribonucleotide). In some embodiments of the assay, upon cleavage of the polynucleotide, the detectable label emits a detectable signal, which is then captured and quantified (e.g., the detectable label is a fluorophore and the detectable signal is fluorescence). Upon detection of a detectable label, it can be determined that the sample from the subject contained target nucleic acids from a coronavirus. In some embodiments, the target nucleic acid comprises the N gene or the E gene of coronavirus and can be assayed for using the compositions and methods disclosed herein. In some embodiments, a DETECTR assay may detect multiple target nucleic acids or amplicons. For example a DETECTR assay may detect multiple target nucleic acids that are specific to SARS-CoV-2, or a DETECTR assay may detect a combination of a target nucleic acid specific to SARS-CoV-2 and a target nucleic acid present in related SARS-like coronaviruses.

The compositions and methods of use thereof disclosed herein include using a programmable nuclease such as a Cas12 protein, a Cas14 protein, or a Cas13 protein to assay for, detect, and/or quantify a nucleic acid from coronavirus (e.g., from a coronavirus such as SARS-CoV-2). In some embodiments, a Cas12 protein, Cas13 protein, or a Cas14 protein is used for detection of a target nucleic acid from coronavirus in a sample from a subject. For example, a Cas12 protein, a Cas14 protein, or a Cas13 protein is complexed with a guide nucleic acid that hybridizes to a target sequence of a target nucleic acid from coronavirus. The complex can be contacted to a sample from a subject. The subject may or may not be infected with coronavirus. For use in an assay with a Cas12 protein, Cas14 protein, or a Cas13 protein, a target nucleic acid in the sample can be reverse transcribed and amplified by thermal (e.g., PCR) or isothermal amplification (e.g., LAMP). For use in an assay with a Cas13 protein, the amplified target nucleic acids can be transcribed back into RNA. If the subject is infected with coronavirus, the guide nucleic acid hybridizes to the target nucleic acid or amplicon thereof leading to activation of the Cas12 protein, Cas14 protein, or Cas13 protein. Upon activation, the Cas12 protein, the Cas14 protein, or the Cas13 protein can cleave a detector nucleic acid, wherein the detector nucleic acid comprises a detectable label attached to the nucleic acid for cleavage by a Cas12 protein, a Cas13 protein, or a Cas14 protein. In some embodiments of the assay, upon cleavage of the detector nucleic acid, the detectable label emits a detectable signal, which can then be captured and quantified (e.g., the detectable label is a fluorophore and the detectable signal is fluorescence). Upon detection of a detectable label, it can be determined that the sample from the subject comprised the target nucleic acids from a coronavirus. In some embodiments, the target nucleic acid comprises the N gene or the E gene of coronavirus and can be assayed for using the compositions and methods disclosed herein.

In some embodiments, a programmable nuclease having at least 60% sequence identity to SEQ ID NO: 28 can be used for detection of a target nucleic acid from coronavirus (e.g., from a coronavirus such as SARS-CoV-2) in a sample from a subject. For example, a programmable nuclease having at least 60% sequence identity to SEQ ID NO: 28 can be complexed with a guide nucleic acid that hybridizes to a target sequence of a target nucleic acid from coronavirus. The complex can be contacted to a sample from a subject. The subject may or may not be infected with coronavirus. The target nucleic acid of the sample can be reverse transcribed and amplified by thermal amplification (e.g., PCR) or by isothermal amplification (e.g., LAMP). If the subject is infected with coronavirus, the guide nucleic acid hybridizes to the target nucleic acid leading to activation of programmable nuclease having at least 60% sequence identity to SEQ ID NO: 28. Upon activation, the programmable nuclease having at least 60% sequence identity to SEQ ID NO: 28 can cleave a detector nucleic acid, wherein the detector nucleic acid comprises a detectable label attached to a nucleic acid. In some embodiments of the assay, upon cleavage of the cleavage, the detectable label emits a detectable signal, which can then be captured and quantified (e.g., the detectable label is a fluorophore and the detectable signal is fluorescence). Upon detection of a detectable label, it can be determined that the sample from the subject contained target nucleic acids from a coronavirus. In some embodiments, the target nucleic acid comprises the N gene or the E gene of coronavirus and can be assayed for using the compositions and methods disclosed herein.

The compositions disclosed herein and methods of use thereof can be used as a companion diagnostic with medicaments used to treat coronavirus, or can be used in reagent kits, point-of-care diagnostics, or over-the-counter diagnostics. The methods may be used as a point of care diagnostic or as a lab test for detection of a target nucleic acid and, thereby, detection of a condition in a subject from which the sample was taken. The methods may be used in various sites or locations, such as in laboratories, in hospitals, in physician offices/laboratories (POLs), in clinics, at remotes sites, or at home. Sometimes, the present disclosure provides various methods, reagents, and devices for consumer genetic use or for over the counter use.

Also described herein are methods, reagents, and devices for detecting the presence of a target nucleic acid in a sample. The methods, reagents, and devices for detecting the presence of a target nucleic acid in a sample can be used in a rapid lab tests for detection of a target nucleic acid of interest (e.g., target nucleic acids from a target population). In particular, provided herein are methods, reagents, and devices wherein the rapid lab tests can be performed in a single system. The target nucleic acid may be a portion of a nucleic acid from a virus (e.g., coronavirus) or other agents responsible for a disease in the sample. The target nucleic acid may be a portion of an RNA or DNA or an amplicon thereof from a coronavirus such as SARS-CoV-2 in the sample.

In some embodiments, programmable nucleases disclosed herein are activated by RNA or DNA to initiate trans cleavage activity of a detector nucleic acid. A programmable nuclease as disclosed herein is, in some cases, binds to a target RNA to initiate trans cleavage of a detector nucleic acid, and this programmable nuclease can be referred to as an RNA-activated programmable RNA nuclease. In some instances, a programmable nuclease as disclosed herein binds to a target DNA to initiate trans cleavage of a detector nucleic acid, and this programmable nuclease can be referred to as a DNA-activated programmable RNA nuclease. In some cases, a programmable nuclease as described herein is capable of being activated by a target RNA or a target DNA. For example, a Cas13 protein, such as Cas13a, disclosed herein is activated by a target RNA nucleic acid or a target DNA nucleic acid to transcollaterally cleave RNA detector nucleic acid. In some embodiments, the Cas13 binds to a target ssDNA which initiates trans cleavage of RNA detector nucleic acid.

The detection of the target nucleic acid in the sample may indicate the presence of the disease in the sample and may provide information for taking action to reduce the transmission of the disease to individuals in the disease-affected environment or near the disease-carrying individual. The detection of the target nucleic acid in the sample may indicate the presence of a disease mutation, such as a single nucleotide polymorphism (SNP) that provide antibiotic resistance to a disease-causing bacteria. The detection of the target nucleic acid is facilitated by a programmable nuclease. The programmable nuclease can become activated after binding of a guide nucleic acid with a target nucleic, in which the activated programmable nuclease can cleave the target nucleic acid and can have trans cleavage activity, which can also be referred to as “collateral” or “transcollateral” cleavage.

Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety is released from the detector nucleic acid and generates a detectable signal that is immobilized to on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or level of the target nucleic acid associated with an ailment, such as a disease. The programmable nuclease can be a CRISPR-Cas (clustered regularly interspaced short palindromic repeats-CRISPR associated) nucleoprotein complex with trans cleavage activity, which can be activated by binding of a guide nucleic acid with a target nucleic acid. These assays, which leverage the transcollateral cleavage properties of CRISPR-Cas enzymes are referred to herein as DNA endonuclease targeted CRISPR trans reporter (DETECTR) reactions. A DETECTR reaction can be performed in a fluidic device.

In some embodiments, the present disclosure provides for Cas12 detection of a target nucleic acid from a coronavirus. In this case, nucleic acids (RNA) from a sample are reverse transcribed and amplified into DNA. Any Cas12 protein disclosed herein is complexed with a guide nucleic acid designed to hybridize to a nucleic acid sequence of the reverse transcribed and amplified DNA. DETECTR reactions are carried out. In the presence of reverse transcribed and amplified DNA indicative of coronavirus, Cas12 is activated to transcollaterally cleave a detector nucleic acid, emitting a detectable signal (e.g., fluorescence). In some embodiments, the present disclosure provides for Cas13 detection of a target nucleic acid from a coronavirus. In this case, RNA in a sample is either directly detected by complexing a Cas13 enzyme with a guide nucleic acid designed to hybridize to a target RNA sequence from a coronavirus or, RNA is reverse transcribed, amplified, and in vitro transcribed prior to contacting it with a Cas13 enzyme complexed with a guide nucleic acid designed to hybridize this amplified target RNA sequence from a coronavirus. In the presence of the RNA (unamplified or amplified), Cas13 is activated to transcollaterally cleave a detector nucleic acid, emitting a detectable signal (e.g., fluorescence).

Also described herein is a kit for detecting a target nucleic acid (e.g., from a coronavirus such as SARS-CoV-2). The kit may comprise a support medium; a guide nucleic acid sequences targeted to a target nucleic acid sequence; a programmable nuclease capable of being activated when complexed with a guide nucleic acid and a target nucleic acid; and a single-stranded detector nucleic acid comprising a detection moiety, wherein the detector nucleic acid is capable of being cleaved by the activated nuclease, thereby generating a first detectable signal.

A biological sample from an individual or an environmental sample can be tested to determine whether the individual has a viral disease (e.g., infected with coronavirus). The at least one target nucleic acid from a target nucleic acid (e.g., from a coronavirus such as SARS-CoV-2) is detected can also indicate that one or more of the target populations is wild-type or comprises a mutation that confers resistance to treatment, such as antibiotic treatment. A sample from an individual or from an environment is applied to the reagents described herein. If the target nucleic acid is present in the sample, the target nucleic acid binds to the guide nucleic acid to activate the programmable nuclease. The activated programmable nuclease cleaves the detector nucleic acid and generates a detectable signal that can be visualized, for example on a support medium. If the target nucleic acid is absent in the sample or below the threshold of detection, the guide nucleic acid remains unbound, the programmable nuclease remains inactivated, and the detector nucleic acid remains uncleaved.

Such methods, reagents, and devices described herein may allow for detection of target nucleic acid, and in turn the disease associated with the target nucleic acids (e.g., coronavirus such as SARS-CoV-2), in remote regions or low resource settings without specialized equipment. Also, such methods, reagents, and devices described herein may allow for detection of target nucleic acid, and in turn the disease associated with the target nucleic acids, in healthcare clinics or doctor offices without specialized equipment. In some cases, this provides a point of care testing for users to quickly and easily test for a disease or infection with high sensitivity at home or in an office of a healthcare provider. Assays that deliver results in under an hour, for example, in 15 to 60 minutes, are particularly desirable for at home testing for many reasons. For example, antivirals can be most effective when administered within the first 48 hours after disease exposure. Thus, the methods disclosed herein, which are capable of delivering results in under an hour, may allow for the delivery of anti-viral therapy during the first 48 hours after infection. Additionally, the systems and assays provided herein, which are capable of delivering quick diagnoses and results, can help keep or send a patient at home, improve comprehensive disease surveillance, and prevent the spread of an infection. In other cases, this provides a test, which can be used in a lab to detect one or more nucleic acid populations or varieties of interest in a sample from a subject. In particular, provided herein are methods, reagents, and devices, wherein the high sensitivity lab tests can be performed in a single assay. In some cases, this may be valuable in detecting diseases in a developing country and as a global healthcare tool to detect the spread of a disease or efficacy of a treatment or provide early detection of a disease.

Some methods as described herein use an editing technique, such as a technique using an editing enzyme or a programmable nuclease and guide nucleic acid, to detect a target nucleic acid (e.g. from a coronavirus such as SARS-CoV-2). An editing enzyme or a programmable nuclease in the editing technique can be activated by one or more target nucleic acids, after which the activated editing enzyme or activated programmable nuclease can cleave nearby single-stranded nucleic acids, such detector nucleic acids with a detection moiety. A target nucleic acid population (e.g., a target nucleic acid from a coronavirus such as SARS-CoV-2), can be amplified by isothermal amplification and then an editing technique can be used to detect the marker. In some instances, the editing technique can comprise an editing enzyme or programmable nuclease that, when activated, cleaves nearby RNA or DNA as the readout of the detection. The methods as described herein in some instances comprise obtaining a cell-free DNA sample, amplifying DNA from the sample, using an editing technique to cleave detector nucleic acids, and reading the output of the editing technique. In other instances, the method comprises obtaining a fluid sample from a patient, and without amplifying a nucleic acid of the fluid sample, using an editing technique to cleave detector nucleic acids, and detecting the nucleic acid. The method can also comprise using single-stranded detector DNA, cleaving the single-stranded detector DNA using an activated editing enzyme, wherein the editing enzyme cleaves at least 50% of a population of single-stranded detector DNA as measured by a change in color. A number of samples, guide nucleic acids, programmable nucleases or editing enzymes, support mediums, target nucleic acids, single-stranded detector nucleic acids, and reagents are consistent with the devices, systems, fluidic devices, kits, and methods disclosed herein.

Also disclosed herein are detector nucleic acids and methods detecting a target nucleic using the detector nucleic acids. Often, the detector nucleic acid is a protein-nucleic acid. For example, a method of assaying for a target nucleic acid (e.g. from a coronavirus such as SARS-CoV-2) in a sample comprises contacting the sample to a plurality of complexes comprising a guide nucleic acid, each guide nucleic acid sequence comprising a segment that is reverse complementary to a segment of a target nucleic acid sequence within a target nucleic acid population and programmable nucleases that exhibits sequence independent cleavage upon forming complexes comprising the segment of the guide nucleic acid binding to the segment of the target nucleic acid; and assaying for a signal indicating cleavage of at least some protein-nucleic acids of a population of protein-nucleic acids, wherein the signal indicates a presence of one or more of the target nucleic acid populations in the sample and wherein absence of the signal indicates an absence of the target nucleic acid population in the sample. Often, the protein-nucleic acid is an enzyme-nucleic acid or an enzyme substrate-nucleic acid. The nucleic acid can be DNA, RNA, or a DNA/RNA hybrid. The methods described herein use a programmable nuclease, such as the CRISPR/Cas system, to detect a target nucleic acid (e.g. from a coronavirus such as SARS-CoV-2). A method of assaying for a target nucleic acid (e.g. from a coronavirus such as SARS-CoV-2) in a sample, for example, comprises: a) contacting the sample to a plurality of complexes comprising a guide nucleic acid, each guide nucleic acid sequence comprising a segment that is reverse complementary to a segment of a nucleic acid target sequence within a target nucleic acid population, and programmable nucleases that exhibits sequence independent cleavage upon forming complexes comprising the segment of the guide nucleic acid binding to the segment of the target nucleic acid; b) contacting the complexes to a substrate; c) contacting the substrate to a reagent that differentially reacts with a cleaved substrate; and d) assaying for a signal indicating cleavage of the substrate, wherein the signal indicates a presence of one or more of the target nucleic acid populations in the sample and wherein absence of the signal indicates an absence of the target nucleic acid population in the sample. Often, the substrate is an enzyme-nucleic acid. Sometimes, the substrate is an enzyme substrate-nucleic acid.

Cleavage of the protein-nucleic acid produces a signal. For example, cleavage of the protein-nucleic acid produces a calorimetric signal, a potentiometric signal, an amperometric signal, an optical signal, or a piezo-electric signal. Various devices can be used to detect these different types signals, which indicate whether a target nucleic acid is present in the sample.

### Sample

A number of samples are consistent with the methods, reagents, and devices disclosed herein.

These samples can comprise a target nucleic acid for detection of an ailment, such as a disease, pathogen, or virus, such as influenza. The pathogen can also be a bacterium, a fungus, a protozoan, or a worm. A pathogen can be a virus, such as coronavirus. Generally, a sample from an individual or an animal or an environmental sample can be obtained to test for presence of a disease, or any mutation of interest. A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated or liquified prior to application to detection system of the present disclosure. A sample from an environment may be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system. Sometimes, the sample is contained in no more 20 μL. The sample, in some cases, is contained in no more than 1, 5, 10, 15, 20, 25, 30, 35 40, 45, 50, 55, 60, 65, 70, 75, 80, 90, 100, 200, 300, 400, 500 μL, or any of value from 1 μL to 500 μL. Sometimes, the sample is contained in more than 500 μL.

In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample used for disease testing may comprise at least one target sequence that can bind to a guide nucleic acid of the reagents described herein. A portion of a nucleic acid can be from a genomic locus, a transcribed mRNA, or a reverse transcribed cDNA. A portion of a nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A portion of a nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target sequence can be reverse complementary to a guide nucleic acid. Each target sequences of the multiple target sequences can be reverse complementary to a distinct guide nucleic acid.

In some cases, the target sequence is a portion of a nucleic acid population from a virus or a bacterium or other agents responsible for a disease in the sample (e.g., a nucleic acid from coronavirus). The target sequence, in some cases, is a portion of a nucleic acid population from a sexually transmitted infection or a contagious disease, in the sample. The target sequence, in some cases, is a portion of a nucleic acid population from an upper respiratory tract infection, a lower respiratory tract infection, or a contagious disease, in the sample. The target sequence, in some cases, is a portion of a nucleic acid population from a hospital acquired infection, or a contagious disease, in the sample. The target sequence, in some cases, is an ssRNA. These target sequences may be from a disease, and the disease may include but is not limited to influenza virus including influenza A virus (IAV) or influenza B virus (IBV), rhinovirus, cold viruses, a respiratory virus, an upper respiratory virus, a lower respiratory virus, or respiratory syncytial virus. Pathogens include viruses, fungi, helminths, protozoa, and parasites. Examples of viruses include coronavirus. All strains of coronavirus can be assayed for using the compositions and methods disclosed herein. For example, the coronavirus can be the SARS-CoV-2. Additionally, the coronavirus can be 229E (alpha coronavirus), NL63 (alpha coronavirus), OC43 (beta coronavirus), HKU1 (beta coronavirus), MERS-CoV, or SARS-CoV. In some embodiments, the compositions and methods disclosed herein specifically target and assay for the SARS-CoV-2 coronavirus. Any nucleic acid of the SARS-CoV-2 can be assayed for using the compositions and methods disclosed herein. In some embodiments, the N gene or the E gene of coronavirus can be assayed for using the compositions and methods disclosed herein. In some embodiments, the guide nucleic acids disclosed herein specifically target and bind a nucleic acid sequence of the SARS-CoV-2 strain. In some embodiments, the guide nucleic acids disclosed herein specifically target and bind the N gene or the E gene. Other pathogens include, e.g., Mycobacterium tuberculosis, Streptococcus agalactiae, methicillin-resistant Staphylococcus aureus, Legionella pneumophila, Streptococcus pyogenes, Escherichia coli, Neisseria meningitidis, Pneumococcus, Hemophilus influenzae B, influenza virus, respiratory syncytial virus (RSV), M. pneumoniae, Streptococcus intermdius, Streptococcus pneumoniae, and Streptococcus pyogenes. Often the target nucleic acid comprises a sequence from a virus or a bacterium or other agents responsible for a disease that can be found in the sample. Pathogenic viruses include but are not limited to influenza virus; RSV; an ssRNA virus, a respiratory virus, an upper respiratory virus, a lower respiratory virus, or a rhinovirus. Pathogens include, e.g., Mycobacterium tuberculosis, Streptococcus agalactiae, Legionella pneumophila, Streptococcus pyogenes, Hemophilus influenzae B influenza virus, respiratory syncytial virus (RSV), or Mycobacterium tuberculosis

The sample can be used for identifying a disease status. For example, a sample is any sample described herein, and is obtained from a subject for use in identifying a disease status (e.g., infected with coronavirus or uninfected) of a subject. Sometimes, a method comprises obtaining a serum sample from a subject; and identifying a disease status of the subject.

In some instances, the target nucleic acid is a single-stranded nucleic acid. Alternatively or in combination, the target nucleic acid is a double stranded nucleic acid and is prepared into single-stranded nucleic acids before or upon contacting the reagents. The target nucleic acid may be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids (e.g., from coronavirus) are consistent with the methods and compositions disclosed herein. Some methods described herein can detect a target nucleic acid present in the sample in various concentrations or amounts as a target nucleic acid. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acids (e.g., from coronavirus) are consistent with the methods and compositions disclosed herein. Some methods described herein can detect two or more target nucleic acid sequences present in the sample in various concentrations or amounts. In some cases, the sample has at least 2 target nucleic acid sequences. In some cases, the sample has at least 3, 4, 5, 6, 7, 8, 9, 10, 20, 30, 40, or 50 target nucleic acid sequences. In some cases, the method detects target nucleic acid sequences that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid sequences can be present at different concentrations or amounts in the sample.

Any of the above disclosed samples are consistent with the systems, assays, and programmable nucleases disclosed herein and can be used as a companion diagnostic with any of the diseases disclosed herein (e.g., a coronavirus infection), or can be used in reagent kits, point-of-care diagnostics, or over-the-counter diagnostics.

### Reagents

A number of reagents are consistent with the methods, reagents, and devices disclosed herein.

These reagents are compatible with the samples, methods, and devices as described herein for detection of an ailment, such as a disease. The reagents described herein for detecting a disease, such as coronavirus, comprise multiple guide nucleic acids, each guide nucleic acid targeting a target nucleic acid segment indicative of the disease. Each guide nucleic acid binds to the target nucleic acid comprising a segment of a nucleic acid sequence (e.g., a nucleic acid from coronavirus) as described herein. Each guide nucleic acid can bind to the target nucleic acid comprising a portion of a nucleic acid (e.g., a target nucleic acid from coronavirus) as described herein and further comprising a mutation, such as a single nucleotide polymorphism (SNP), that can confer resistance to a treatment, such as antibiotic treatment. Each guide nucleic acid binds to the target nucleic acid comprising a portion of a nucleic acid. Each guide nucleic acid is complementary to a target nucleic acid. Often the guide nucleic acid binds specifically to the target nucleic acid. The target nucleic acid may be a RNA, DNA, or synthetic nucleic acids.

Disclosed herein are methods of assaying for a plurality of target nucleic acids (e.g., a plurality of nucleic acids from coronavirus) as described herein. For example, a method of assaying for a plurality of target nucleic acids in a sample comprises contacting the sample to a complex comprising a plurality guide nucleic acid sequences, each guide nucleic acid sequence comprising a segment that is reverse complementary to a segment of the target nucleic acid, and programmable nucleases that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of one or more target nucleic acid of the plurality of target nucleic acids in the sample and wherein absence of the signal indicates an absence of the target nucleic acids in the sample. As another example, a method of assaying for a target nucleic acid in a sample, for example, comprises: a) contacting the sample to a plurality of complexes, each complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the plurality of complexes to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. A signal can be a calorimetric, potentiometric, amperometric, optical (e.g., fluorescent, colorometric, etc.), or piezo-electric signal. Often, the signal is present prior to detector nucleic acid cleavage and changes upon detector nucleic acid cleavage. Sometimes, the signal is absent prior to detector nucleic acid cleavage and is present upon detector nucleic acid cleavage. The detectable signal can be immobilized on a support medium for detection. The programmable nuclease can be a CRISPR-Cas (clustered regularly interspaced short palindromic repeats-CRISPR associated) nucleoprotein complex with trans cleavage activity, which can be activated by binding of a guide nucleic acid with a target nucleic acid. The CRISPR-Cas nucleoprotein complex can comprise a Cas protein (also referred to as a Cas nuclease) complexed with a guide nucleic acid, which can also be referred to as CRISPR enzyme. A guide nucleic acid can be a CRISPR RNA (crRNA). Sometimes, a guide nucleic acid comprises a crRNA and a trans-activating crRNA (tracrRNA).

The CRISPR/Cas system used to detect a modified target nucleic acids can comprise CRISPR RNAs (crRNAs), trans-activating crRNAs (tracrRNAs), Cas proteins, and detector nucleic acids.

A guide nucleic acid can comprise a sequence that is reverse complementary to the sequence of a target nucleic acid. A guide nucleic acid can be a crRNA. Sometimes, a guide nucleic acid comprises a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. It is understood that the sequence of a polynucleotide need not be 100% complementary to that of its target nucleic acid to be specifically hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid sequence of a strain of an infection or genomic locus of interest. The guide nucleic acid can be selected from a group of guide nucleic acids that have been tiled against the nucleic acid sequence of a strain of coronavirus. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances the tiling of the guide nucleic acids is non-sequential. Often, a method for detecting a target nucleic acid comprises contacting a target nucleic acid to a pool of guide nucleic acids and a programmable nuclease, wherein a guide nucleic acid of the pool of guide nucleic acids has a sequence selected from a group of tiled guide nucleic acid that correspond to nucleic acids of a target nucleic acid; and assaying for a signal produce by cleavage of at least some detector nucleic acids of a population of detector nucleic acids. Pooling of guide nucleic acids can ensure broad spectrum identification, or broad coverage, of a target species within a single reaction. This can be particularly helpful in diseases or indications, like sepsis, that may be caused by multiple organisms. In some embodiments, the guide pooling comprises guide nucleic acids that produce the best signal in a DETECTR reaction (e.g., top 10 gRNAs). In some embodiments, there is an increased signal to noise ratio as the number of pooled gRNAs increases (e.g., signal to noise for 1 gRNA<2 pooled gRNAs<3 pooled gRNAs<4 pooled gRNAs<5 pooled gRNAs<6 pooled gRNAs<7 pooled gRNAs<8 pooled gRNAs<9 pooled gRNAs<10 pooled gRNAs).

Described herein are reagents comprising a programmable nuclease capable of being activated when complexed with the guide nucleic acid and the target nucleic acid segment. A programmable nuclease can be capable of being activated when complexed with a guide nucleic acid and the target sequence. The programmable nuclease can be activated upon binding of the guide nucleic acid to its target nucleic acid and degrades non-specifically nucleic acid in its environment. The programmable nuclease has trans cleavage activity once activated. A programmable nuclease can be a Cas protein (also referred to, interchangeably, as a Cas nuclease). A crRNA and Cas protein can form a CRISPR enzyme.

“Percent identity” and “% identity” can refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

Several programmable nucleases are consistent with the methods and devices of the present disclosure. For example, CRISPR/Cas enzymes are programmable nucleases used in the methods and systems disclosed herein. CRISPR/Cas enzymes can include any of the known Classes and Types of CRISPR/Cas enzymes. Programmable nucleases disclosed herein include Class 1 CRISPR/Cas enzymes, such as the Type I, Type IV, or Type III CRISPR/Cas enzymes. Programmable nucleases disclosed herein also include the Class 2 CRISPR/Cas enzymes, such as the Type II, Type V, and Type VI CRISPR/Cas enzymes. Preferable programmable nucleases included in the several assays disclosed herein (e.g., for assaying for coronavirus in a device, such as a microfluidic device or a lateral flow assay) and methods of use thereof include a Type V or Type VI CRISPR/Cas enzyme.

A programmable nuclease of the present disclosure may be configured to form a multimeric complex with target nucleic acid binding affinity. A programmable nuclease of the present disclosure may form a homodimeric complex (e.g., two proteins comprising identical sequences non-covalently associate to form an optionally catalytically active complex) or a heterodimeric complex (e.g., two proteins comprising different sequences non-covalently associate to form an optionally catalytically active complex).

In some embodiments, the Type V CRISPR/Cas enzyme is a programmable Cas12 nuclease. Type V CRISPR/Cas enzymes (e.g., Cas12 or Cas14) lack an HNH domain. A Cas12 nuclease of the present disclosure cleaves a nucleic acids via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. (Murugan et al., Mol Cell. 2017 Oct. 5; 68(1): 15-25). A programmable Cas12 nuclease can be a Cas12a (also referred to as Cpf1) protein, a Cas12b protein, Cas12c protein, Cas12d protein, or a Cas12e protein. In some cases, a suitable Cas12 protein comprises an amino acid sequence having at least 80%, at least 85%, at least 90%, at least 95%, at least 98%, at least 99%, or 100%, amino acid sequence identity to any one of SEQ ID NO: 18-SEQ ID NO: 60.

TABLE 1
Cas12 Protein Sequences

| SEQ | | |
|---|---|---|
| ID | | |
| NO | Description | Sequence |
| SEQ | Lachnospiraceae | MSKLEKFTNCYSLSKTLRFKAIPVGKTQENIDNKRLLVEDEKRAEDYK |
| ID | bacterium | GVKKLLDRYYLSFINDVLHSIKLKNLNNYISLFRKKTRTEKENKELENL |
| NO: | ND2006 | EINLRKEIAKAFKGNEGYKSLFKKDIIETILPEFLDDKDEIALVNSFNGFT |
| 18 | (LbCas12a) | TAFTGFFDNRENMFSEEAKSTSIAFRCINENLTRYISNMDIFEKVDAIFDK |
| | | HEVQEIKEKILNSDYDVEDFFEGEFFNFVLTQEGIDVYNAIIGGFVTESG |
| | | EKIKGLNEYINLYNQKTKQKLPKFKPLYKQVLSDRESLSFYGEGYTSDE |
| | | EVLEVFRNTLNKNSEIFSSIKKLEKLFKNFDEYSSAGIFVKNGPAISTISK |
| | | DIFGEWNVIRDKWNAEYDDIHLKKKAVVTEKYEDDRRKSFKKIGSFSL |
| | | EQLQEYADADLSVVEKLKEIIIQKVDEIYKVYGSSEKLFDADFVLEKSL |
| | | KKNDAVVAIMKDLLDSVKSFENYIKAFFGEGKETNRDESFYGDFVLAY |
| | | DILLKVDHIYDAIRNYVTQKPYSKDKFKLYFQNPQFMGGWDKDKETD |
| | | YRATILRYGSKYYLAIMDKKYAKCLQKIDKDDVNGNYEKINYKLLPGP |
| | | NKMLPKVFFSKKWMAYYNPSEDIQKIYKNGTFKKGDMFNLNDCHKLI |
| | | DFFKDSISRYPKWSNAYDFNFSETEKYKDIAGFYREVEEQGYKVSFESA |
| | | SKKEVDKLVEEGKLYMFQIYNKDFSDKSHGTPNLHTMYFKLLFDENNH |
| | | GQIRLSGGAELFMRRASLKKEELVVHPANSPIANKNPDNPKKTTTLSYD |
| | | VYKDKRFSEDQYELHIPIAINKCPKNIFKINTEVRVLLKHDDNPYVIGID |
| | | RGERNLLYIVVVDGKGNIVEQYSLNEIINNFNGIRIKTDYHSLLDKKEKE |
| | | RFEARQNWTSIENIKELKAGYISQVVHKICELVEKYDAVIALEDLNSGF |
| | | KNSRVKVEKQVYQKFEKMLIDKLNYMVDKKSNPCATGGALKGYQITN |
| | | KFESFKSMSTQNGFIFYIPAWLTSKIDPSTGFVNLLKTKYTSIADSKKFIS |
| | | SFDRIMYVPEEDLFEFALDYKNFSRTDADYIKKWKLYSYGNRIRIFRNP |
| | | KKNNVFDWEEVCLTSAYKELFNKYGINYQQGDIRALLCEQSDKAFYSS |
| | | FMALMSLMLQMRNSITGRTDVDFLISPVKNSDGIFYDSRNYEAQENAIL |
| | | PKNADANGAYNIARKVLWAIGQFKKAEDEKLDKVKIAISNKEWLEYA |
| | | QTSVKH |
| SEQ | Acidaminococcus | MTQFEGFTNLYQVSKTLRFELIPQGKTLKHIQEQGFIEEDKARNDHYKE |
| ID | sp. | LKPIIDRIYKTYADQCLQLVQLDWENLSAAIDSYRKEKTEETRNALIEEQ |
| NO: | BV316 | ATYRNAIHDYFIGRTDNLTDAINKRHAEIYKGLFKAELFNGKVLKQLGT |
| 19 | (AsCas12a) | VTTTEHENALLRSFDKFTTYFSGFYENRKNVFSAEDISTAIPHRIVQDNF |
| | | PKFKENCHIFTRLITAVPSLREHFENVKKAIGIFVSTSIEEVFSFPFYNQLL |
| | | TQTQIDLYNQLLGGISREAGTEKIKGLNEVLNLAIQKNDETAHIIASLPH |
| | | RFIPLFKQILSDRNTLSFILEEFKSDEEVIQSFCKYKTLLRNENVLETAEA |
| | | LFNELNSIDLTHIFISHKKLETISSALCDHWDTLRNALYERRISELTGKIT |
| | | KSAKEKVQRSLKHEDINLQEIISAAGKELSEAFKQKTSEILSHAHAALDQ |
| | | PLPTTLKKQEEKEILKSQLDSLLGLYHLLDWFAVDESNEVDPEFSARLT |
| | | GIKLEMEPSLSFYNKARNYATKKPYSVEKFKLNFQMPTLASGWDVNKE |
| | | KNNGAILFVKNGLYYLGIMPKQKGRYKALSFEPTEKTSEGFDKMYYDY |
| | | FPDAAKMIPKCSTQLKAVTAHFQTHTTPILLSNNFIEPLEITKEIYDLNNP |
| | | EKEPKKFQTAYAKKTGDQKGYREALCKWIDFTRDFLSKYTKTTSIDLSS |
| | | LRPSSQYKDLGEYYAELNPLLYHISFQRIAEKEIMDAVETGKLYLFQIYN |
| | | KDFAKGHHGKPNLHTLYWTGLFSPENLAKTSIKLNGQAELFYRPKSRM |
| | | KRMAHRLGEKMLNKKLKDQKTPIPDTLYQELYDYVNHRLSHDLSDEA |
| | | RALLPNVITKEVSHEIIKDRRFTSDKFFFHVPITLNYQAANSPSKFNQRV |
| | | NAYLKEHPETPIIGIDRGERNLIYITVIDSTGKILEQRSLNTIQQFDYQKKL |
| | | DNREKERVAARQAWSVVGTIKDLKQGYLSQVIHEIVDLMIHYQAVVV |
| | | LENLNFGFKSKRTGIAEKAVYQQFEKMLIDKLNCLVLKDYPAEKVGGV |
| | | LNPYQLTDQFTSFAKMGTQSGFLFYVPAPYTSKIDPLTGFVDPFVWKTI |
| | | KNHESRKHFLEGFDFLHYDVKTGDFILHFKMNRNLSFQRGLPGFMPAW |
| | | DIVFEKNETQFDAKGTPFIAGKRIVPVIENHRFTGRYRDLYPANELIALL |
| | | EEKGIVFRDGSNILPKLLENDDSHAIDTMVALIRSVLQMRNSNAATGED |
| | | YINSPVRDLNGVCFDSRFQNPEWPMDADANGAYHIALKGQLLLNHLKE |
| | | SKDLKLQNGISNQDWLAYIQELRN |
| SEQ | Francisella | MSIYQEFVNKYSLSKTLRFELIPQGKTLENIKARGLILDDEKRAKDYKK |
| ID | novicida | AKQIIDKYHQFFIEEILSSVCISEDLLQNYSDVYFKLKKSDDDNLQKDFK |
| NO: | U112 | SAKDTIKKQISEYIKDSEKFKNLFNQNLIDAKKGQESDLILWLKQSKDN |
| 20 | (FnCas12a) | GIELFKANSDITDIDEALEIIKSFKGWTTYFKGFHENRKNVYSSNDIPTSII |
| | | YRIVDDNLPKFLENKAKYESLKDKAPEAINYEQIKKDLAEELTFDIDYK |
| | | TSEVNQRVFSLDEVFEIANFNNYLNQSGITKFNTIIGGKFVNGENTKRKG |
| | | INEYINLYSQQINDKTLKKYKMSVLFKQILSDTESKSFVIDKLEDDSDVV |
| | | TTMQSFYEQIAAFKTVEEKSIKETLSLLFDDLKAQKLDLSKIYFKNDKSL |
| | | TDLSQQVFDDYSVIGTAVLEYITQQIAPKNLDNPSKKEQELIAKKTEKA |
| | | KYLSLETIKLALEEFNKHRDIDKQCRFEEILANFAAIPMIFDEIAQNKDNL |
| | | AQISIKYQNQGKKDLLQASAEDDVKAIKDLLDQTNNLLHKLKIFHISQS |
| | | EDKANILDKDEHFYLVFEECYFELANIVPLYNKIRNYITQKPYSDEKFKL |
| | | NFENSTLANGWDKNKEPDNTAILFIKDDKYYLGVMNKKNNKIFDDKAI |
| | | KENKGEGYKKIVYKLLPGANKMLPKVFFSAKSIKFYNPSEDILRIRNHST |
| | | HTKNGSPQKGYEKFEFNIEDCRKFIDFYKQSISKHPEWKDFGFRFSDTQ |
| | | RYNSIDEFYREVENQGYKLTFENISESYIDSVVNQGKLYLFQIYNKDFSA |
| | | YSKGRPNLHTLYWKALFDERNLQDVVYKLNGEAELFYRKQSIPKKITH |
| | | PAKEAIANKNKDNPKKESVFEYDLIKDKRFTEDKFFFHCPITINFKSSGA |
| | | NKFNDEINLLLKEKANDVHILSIDRGERHLAYYTLVDGKGNIIKQDTFNI |
| | | IGNDRMKTNYHDKLAAIEKDRDSARKDWKKINNIKEMKEGYLSQVVH |
| | | EIAKLVIEYNAIVVFEDLNFGFKRGRFKVEKQVYQKLEKMLIEKLNYLV |
| | | FKDNEFDKTGGVLRAYQLTAPFETFKKMGKQTGIIYYVPAGFTSKICPV |
| | | TGFVNQLYPKYESVSKSQEFFSKFDKICYNLDKGYFEFSFDYKNFGDKA |
| | | AKGKWTIASFGSRLINFRNSDKNHNWDTREVYPTKELEKLLKDYSIEY |
| | | GHGECIKAAICGESDKKFFAKLTSVLNTILQMRNSKTGTELDYLISPVAD |
| | | VNGNFFDSRQAPKNMPQDADANGAYHIGLKGLMLLGRIKNNQEGKKL |
| | | NLVIKNEEYFEFVQNRNN |
| SEQ | Porphyromonas | MKTQHFFEDFTSLYSLSKTIRFELKPIGKTLENIKKNGLIRRDEQRLDDY |
| ID | macacae | EKLKKVIDEYHEDFIANILSSFSFSEEILQSYIQNLSESEARAKIEKTMRDT |
| NO: | (PmCas12a) | LAKAFSEDERYKSIFKKELVKKDIPVWCPAYKSLCKKFDNFTTSLVPFH |
| 21 | | ENRKNLYTSNEITASIPYRIVHVNLPKFIQNIEALCELQKKMGADLYLEM |
| | | MENLRNVWPSFVKTPDDLCNLKTYNHLMVQSSISEYNRFVGGYSTEDG |
| | | TKHQGINEWINIYRQRNKEMRLPGLVFLHKQILAKVDSSSFISDTLEND |
| | | DQVFCVLRQFRKLFWNTVSSKEDDAASLKDLFCGLSGYDPEAIYVSDA |
| | | HLATISKNIFDRWNYISDAIRRKTEVLMPRKKESVERYAEKISKQIKKRQ |
| | | SYSLAELDDLLAHYSEESLPAGFSLLSYFTSLGGQKYLVSDGEVILYEEG |
| | | SNIWDEVLIAFRDLQVILDKDFTEKKLGKDEEAVSVIKKALDSALRLRK |
| | | FFDLLSGTGAEIRRDSSFYALYTDRMDKLKGLLKMYDKVRNYLTKKPY |
| | | SIEKFKLHFDNPSLLSGWDKNKELNNLSVIFRQNGYYYLGIMTPKGKNL |
| | | FKTLPKLGAEEMFYEKMEYKQIAEPMLMLPKVFFPKKTKPAFAPDQSV |
| | | VDIYNKKTFKTGQKGFNKKDLYRLIDFYKEALTVHEWKLFNFSFSPTEQ |
| | | YRNIGEFFDEVREQAYKVSMVNVPASYIDEAVENGKLYLFQIYNKDFSP |
| | | YSKGIPNLHTLYWKALFSEQNQSRVYKLCGGGELFYRKASLHMQDTT |
| | | VHPKGISIHKKNLNKKGETSLFNYDLVKDKRFTEDKFFFHVPISINYKNK |
| | | KITNVNQMVRDYIAQNDDLQIIGIDRGERNLLYISRIDTRGNLLEQFSLN |
| | | VIESDKGDLRTDYQKILGDREQERLRRRQEWKSIESIKDLKDGYMSQV |
| | | VHKICNMVVEHKAIVVLENLNLSFMKGRKKVEKSVYEKFERMLVDKL |
| | | NYLVVDKKNLSNEPGGLYAAYQLTNPLFSFEELHRYPQSGILFFVDPW |
| | | NTSLTDPSTGFVNLLGRINYTNVGDARKFFDRFNAIRYDGKGNILFDLD |
| | | LSRFDVRVETQRKLWTLTTFGSRIAKSKKSGKWMVERIENLSLCFLELF |
| | | EQFNIGYRVEKDLKKAILSQDRKEFYVRLIYLFNLMMQIRNSDGEEDYI |
| | | LSPALNEKNLQFDSRLIEAKDLPVDADANGAYNVARKGLMVVQRIKR |
| | | GDHESIHRIGRAQWLRYVQEGIVE |
| SEQ | Moraxella | MLFQDFTHLYPLSKTVRFELKPIDRTLEHIHAKNFLSQDETMADMHQK |
| ID | bovoculi | VKVILDDYHRDFIADMMGEVKLTKLAEFYDVYLKFRKNPKDDELQKQ |
| NO: | 237 | LKDLQAVLRKEIVKPIGNGGKYKAGYDRLFGAKLFKDGKELGDLAKF |
| 22 | (MbCas12a) | VIAQEGESSPKLAHLAHFEKFSTYFTGFHDNRKNMYSDEDKHTAIAYRL |
| | | IHENLPRFIDNLQILTTIKQKHSALYDQIINELTASGLDVSLASHLDGYHK |
| | | LLTQEGITAYNTLLGGISGEAGSPKIQGINELINSHHNQHCHKSERIAKLR |
| | | PLHKQILSDGMSVSFLPSKFADDSEMCQAVNEFYRHYADVFAKVQSLF |
| | | DGFDDHQKDGIYVEHKNLNELSKQAFGDFALLGRVLDGYYVDVVNPE |
| | | FNERFAKAKTDNAKAKLTKEKDKFIKGVHSLASLEQAIEHYTARHDDE |
| | | SVQAGKLGQYFKHGLAGVDNPIQKIHNNHSTIKGFLERERPAGERALPK |
| | | IKSGKNPEMTQLRQLKELLDNALNVAHFAKLLTTKTTLDNQDGNFYGE |
| | | FGVLYDELAKIPTLYNKVRDYLSQKPFSTEKYKLNFGNPTLLNGWDLN |
| | | KEKDNFGVILQKDGCYYLALLDKAHKKVFDNAPNTGKSIYQKMIYKY |
| | | LEVRKQFPKVFFSKEAIAINYHPSKELVEIKDKGRQRSDDERLKLYRFIL |
| | | ECLKIHPKYDKKFEGAIGDIQLFKKDKKGREVPISEKDLFDKINGIFSSKP |
| | | KLEMEDFFIGEFKRYNPSQDLVDQYNIYKKIDSNDNRKKENFYNNHPK |
| | | FKKDLVRYYYESMCKHEEWEESFEFSKKLQDIGCYVDVNELFTEIETRR |
| | | LNYKISFCNINADYIDELVEQGQLYLFQIYNKDFSPKAHGKPNLHTLYF |
| | | KALFSEDNLADPIYKLNGEAQIFYRKASLDMNETTIHRAGEVLENKNPD |
| | | NPKKRQFVYDIIKDKRYTQDKFMLHVPITMNFGVQGMTIKEFNKKVNQ |
| | | SIQQYDEVNVIGIDRGERHLLYLTVINSKGEILEQCSLNDITTASANGTQ |
| | | MTTPYHKILDKREIERLNARVGWGEIETIKELKSGYLSHVVHQISQLML |
| | | KYNAIVVLEDLNFGFKRGRFKVEKQIYQNFENALIKKLNHLVLKDKAD |
| | | DEIGSYKNALQLTNNFTDLKSIGKQTGFLFYVPAWNTSKIDPETGFVDL |
| | | LKPRYENIAQSQAFFGKFDKICYNADKDYFEFHIDYAKFTDKAKNSRQI |
| | | WTICSHGDKRYVYDKTANQNKGAAKGINVNDELKSLFARHHINEKQP |
| | | NLVMDICQNNDKEFHKSLMYLLKTLLALRYSNASSDEDFILSPVANDE |
| | | GVFFNSALADDTQPQNADANGAYHIALKGLWLLNELKNSDDLNKVKL |
| | | AIDNQTWLNFAQNR |
| SEQ | Moraxella | MGIHGVPAALFQDFTHLYPLSKTVRFELKPIGRTLEHIHAKNFLSQDET |
| ID | bovoculi | MADMYQKVKVILDDYHRDFIADMMGEVKLTKLAEFYDVYLKFRKNP |
| NO: | AAX08_00205 | KDDGLQKQLKDLQAVLRKESVKPIGSGGKYKTGYDRLFGAKLFKDGK |
| 23 | (Mb2Cas12a) | ELGDLAKFVIAQEGESSPKLAHLAHFEKFSTYFTGFHDNRKNMYSDED |
| | | KHTAIAYRLIHENLPRFIDNLQILTTIKQKHSALYDQIINELTASGLDVSL |
| | | ASHLDGYHKLLTQEGITAYNRIIGEVNGYTNKHNQICHKSERIAKLRPL |
| | | HKQILSDGMGVSFLPSKFADDSEMCQAVNEFYRHYTDVFAKVQSLFDG |
| | | FDDHQKDGIYVEHKNLNELSKQAFGDFALLGRVLDGYYVDVVNPEFN |
| | | ERFAKAKTDNAKAKLTKEKDKFIKGVHSLASLEQAIEHHTARHDDESV |
| | | QAGKLGQYFKHGLAGVDNPIQKIHNNHSTIKGFLERERPAGERALPKIK |
| | | SGKNPEMTQLRQLKELLDNALNVAHFAKLLTTKTTLDNQDGNFYGEF |
| | | GVLYDELAKIPTLYNKVRDYLSQKPFSTEKYKLNFGNPTLLNGWDLNK |
| | | EKDNFGVILQKDGCYYLALLDKAHKKVFDNAPNTGKNVYQKMVYKL |
| | | LPGPNKMLPKVFFAKSNLDYYNPSAELLDKYAKGTHKKGDNFNLKDC |
| | | HALIDFFKAGINKHPEWQHFGFKFSPTSSYRDLSDFYREVEPQGYQVKF |
| | | VDINADYIDELVEQGKLYLFQIYNKDFSPKAHGKPNLHTLYFKALFSED |
| | | NLADPIYKLNGEAQIFYRKASLDMNETTIHRAGEVLENKNPDNPKKRQ |
| | | FVYDIIKDKRYTQDKFMLHVPITMNFGVQGMTIKEFNKKVNQSIQQYD |
| | | EVNVIGIDRGERHLLYLTVINSKGEILEQRSLNDITTASANGTQVTTPYH |
| | | KILDKREIERLNARVGWGEIETIKELKSGYLSHVVHQINQLMLKYNAIV |
| | | VLEDLNFGFKRGRFKVEKQIYQNFENALIKKLNHLVLKDKADDEIGSY |
| | | KNALQLTNNFTDLKSIGKQTGFLFYVPAWNTSKIDPETGFVDLLKPRYE |
| | | NIAQSQAFFGKFDKICYNTDKGYFEFHIDYAKFTDKAKNSRQKWAICSH |
| | | GDKRYVYDKTANQNKGAAKGINVNDELKSLFARYHINDKQPNLVMDI |
| | | CQNNDKEFHKSLMCLLKTLLALRYSNASSDEDFILSPVANDEGVFFNSA |
| | | LADDTQPQNADANGAYHIALKGLWLLNELKNSDDLNKVKLAIDNQTW |
| | | LNFAQNR |
| SEQ | Moraxella | MGIHGVPAALFQDFTHLYPLSKTVRFELKPIGKTLEHIHAKNFLNQDET |
| ID | bovoculi | MADMYQKVKAILDDYHRDFIADMMGEVKLTKLAEFYDVYLKFRKNP |
| NO: | AAX11_00205 | KDDGLQKQLKDLQAVLRKEIVKPIGNGGKYKAGYDRLFGAKLFKDGK |
| 24 | (Mb3Cas12a) | ELGDLAKFVIAQEGESSPKLAHLAHFEKFSTYFTGFHDNRKNMYSDED |
| | | KHTAIAYRLIHENLPRFIDNLQILATIKQKHSALYDQIINELTASGLDVSL |
| | | ASHLDGYHKLLTQEGITAYNTLLGGISGEAGSRKIQGINELINSHHNQHC |
| | | HKSERIAKLRPLHKQILSDGMGVSFLPSKFADDSEVCQAVNEFYRHYA |
| | | DVFAKVQSLFDGFDDYQKDGIYVEYKNLNELSKQAFGDFALLGRVLD |
| | | GYYVDVVNPEFNERFAKAKTDNAKAKLTKEKDKFIKGVHSLASLEQAI |
| | | EHYTARHDDESVQAGKLGQYFKHGLAGVDNPIQKIHNNHSTIKGFLER |
| | | ERPAGERALPKIKSDKSPEIRQLKELLDNALNVAHFAKLLTTKTTLHNQ |
| | | DGNFYGEFGALYDELAKIATLYNKVRDYLSQKPFSTEKYKLNFGNPTL |
| | | LNGWDLNKEKDNFGVILQKDGCYYLALLDKAHKKVFDNAPNTGKSV |
| | | YQKMIYKLLPGPNKMLPKVFFAKSNLDYYNPSAELLDKYAQGTHKKG |
| | | DNFNLKDCHALIDFFKAGINKHPEWQHFGFKFSPTSSYQDLSDFYREVE |
| | | PQGYQVKFVDINADYINELVEQGQLYLFQIYNKDFSPKAHGKPNLHTL |
| | | YFKALFSEDNLVNPIYKLNGEAEIFYRKASLDMNETTIHRAGEVLENKN |
| | | PDNPKKRQFVYDIIKDKRYTQDKFMLHVPITMNFGVQGMTIKEFNKKV |
| | | NQSIQQYDEVNVIGIDRGERHLLYLTVINSKGEILEQRSLNDITTASANG |
| | | TQMTTPYHKILDKREIERLNARVGWGEIETIKELKSGYLSHVVHQISQL |
| | | MLKYNAIVVLEDLNFGFKRGRFKVEKQIYQNFENALIKKLNHLVLKDK |
| | | ADDEIGSYKNALQLTNNFTDLKSIGKQTGFLFYVPAWNTSKIDPETGFV |
| | | DLLKPRYENIAQSQAFFGKFDKICYNADRGYFEFHIDYAKFNDKAKNSR |
| | | QIWKICSHGDKRYVYDKTANQNKGATIGVNVNDELKSLFTRYHINDKQ |
| | | PNLVMDICQNNDKEFHKSLMYLLKTLLALRYSNASSDEDFILSPVANDE |
| | | GVFFNSALADDTQPQNADANGAYHIALKGLWLLNELKNSDDLNKVKL |
| | | AIDNQTWLNFAQNR |
| SEQ | Thiomicrospira | MGIHGVPAATKTFDSEFFNLYSLQKTVRFELKPVGETASFVEDFKNEGL |
| ID | sp. XS5 | KRVVSEDERRAVDYQKVKEIIDDYHRDFIEESLNYFPEQVSKDALEQAF |
| NO: | (TsCas12a) | HLYQKLKAAKVEEREKALKEWEALQKKLREKVVKCFSDSNKARFSRI |
| 25 | | DKKELIKEDLINWLVAQNREDDIPTVETFNNFTTYFTGFHENRKNIYSK |
| | | DDHATAISFRLIHENLPKFFDNVISFNKLKEGFPELKFDKVKEDLEVDYD |
| | | LKHAFEIEYFVNFVTQAGIDQYNYLLGGKTLEDGTKKQGMNEQINLFK |
| | | QQQTRDKARQIPKLIPLFKQILSERTESQSFIPKQFESDQELFDSLQKLHN |
| | | NCQDKFTVLQQAILGLAEADLKKVFIKTSDLNALSNTIFGNYSVFSDAL |
| | | NLYKESLKTKKAQEAFEKLPAHSIHDLIQYLEQFNSSLDAEKQQSTDTV |
| | | LNYFIKTDELYSRFIKSTSEAFTQVQPLFELEALSSKRRPPESEDEGAKGQ |
| | | EGFEQIKRIKAYLDTLMEAVHFAKPLYLVKGRKMIEGLDKDQSFYEAF |
| | | EMAYQELESLIIPIYNKARSYLSRKPFKADKFKINFDNNTLLSGWDANK |
| | | ETANASILFKKDGLYYLGIMPKGKTFLFDYFVSSEDSEKLKQRRQKTAE |
| | | EALAQDGESYFEKIRYKLLPGASKMLPKVFFSNKNIGFYNPSDDILRIRN |
| | | TASHTKNGTPQKGHSKVEFNLNDCHKMIDFFKSSIQKHPEWGSFGFTFS |
| | | DTSDFEDMSAFYREVENQGYVISFDKIKETYIQSQVEQGNLYLFQIYNK |
| | | DFSPYSKGKPNLHTLYWKALFEEANLNNVVAKLNGEAEIFFRRHSIKAS |
| | | DKVVHPANQAIDNKNPHTEKTQSTFEYDLVKDKRYTQDKFFFHVPISL |
| | | NFKAQGVSKFNDKVNGFLKGNPDVNIIGIDRGERHLLYFTVVNQKGEIL |
| | | VQESLNTLMSDKGHVNDYQQKLDKKEQERDAARKSWTTVENIKELKE |
| | | GYLSHVVHKLAHLIIKYNAIVCLEDLNFGFKRGRFKVEKQVYQKFEKA |
| | | LIDKLNYLVFKEKELGEVGHYLTAYQLTAPFESFKKLGKQSGILFYVPA |
| | | DYTSKIDPTTGFVNFLDLRYQSVEKAKQLLSDFNAIRFNSVQNYFEFEID |
| | | YKKLTPKRKVGTQSKWVICTYGDVRYQNRRNQKGHWETEEVNVTEK |
| | | LKALFASDSKTTTVIDYANDDNLIDVILEQDKASFFKELLWLLKLTMTL |
| | | RHSKIKSEDDFILSPVKNEQGEFYDSRKAGEVWPKDADANGAYHIALK |
| | | GLWNLQQINQWEKGKTLNLAIKNQDWFSFIQEKPYQE |
| SEQ | Butyrivibrio | MGIHGVPAAYYQNLTKKYPVSKTIRNELIPIGKTLENIRKNNILESDVKR |
| ID | sp. NC3005 | KQDYEHVKGIMDEYHKQLINEALDNYMLPSLNQAAEIYLKKHVDVED |
| NO: | (BsCas12a) | REEFKKTQDLLRREVTGRLKEHENYTKIGKKDILDLLEKLPSISEEDYNA |
| 26 | | LESFRNFYTYFTSYNKVRENLYSDEEKSSTVAYRLINENLPKFLDNIKSY |
| | | AFVKAAGVLADCIEEEEQDALFMVETFNMTLTQEGIDMYNYQIGKVNS |
| | | AINLYNQKNHKVEEFKKIPKMKVLYKQILSDREEVFIGEFKDDETLLSSI |
| | | GAYGNVLMTYLKSEKINIFFDALRESEGKNVYVKNDLSKTTMSNIVFGS |
| | | WSAFDELLNQEYDLANENKKKDDKYFEKRQKELKKNKSYTLEQMSNL |
| | | SKEDISPIENYIERISEDIEKICIYNGEFEKIVVNEHDSSRKLSKNIKAVKVI |
| | | KDYLDSIKELEHDIKLINGSGQELEKNLVVYVGQEEALEQLRPVDSLYN |
| | | LTRNYLTKKPFSTEKVKLNFNKSTLLNGWDKNKETDNLGILFFKDGKY |
| | | YLGIMNTTANKAFVNPPAAKTENVFKKVDYKLLPGSNKMLPKVFFAKS |
| | | NIGYYNPSTELYSNYKKGTHKKGPSFSIDDCHNLIDFFKESIKKHEDWS |
| | | KFGFEFSDTADYRDISEFYREVEKQGYKLTFTDIDESYINDLIEKNELYL |
| | | FQIYNKDFSEYSKGKLNLHTLYFMMLFDQRNLDNVVYKLNGEAEVFY |
| | | RPASIAENELVIHKAGEGIKNKNPNRAKVKETSTFSYDIVKDKRYSKYK |
| | | FTLHIPITMNFGVDEVRRFNDVINNALRTDDNVNVIGIDRGERNLLYVV |
| | | VINSEGKILEQISLNSIINKEYDIETNYHALLDEREDDRNKARKDWNTIE |
| | | NIKELKTGYLSQVVNVVAKLVLKYNAIICLEDLNFGFKRGRQKVEKQV |
| | | YQKFEKMLIEKLNYLVIDKSREQVSPEKMGGALNALQLTSKFKSFAEL |
| | | GKQSGIIYYVPAYLTSKIDPTTGFVNLFYIKYENIEKAKQFFDGFDFIRFN |
| | | KKDDMFEFSFDYKSFTQKACGIRSKWIVYTNGERIIKYPNPEKNNLFDE |
| | | KVINVTDEIKGLFKQYRIPYENGEDIKEIIISKAEADFYKRLFRLLHQTLQ |
| | | MRNSTSDGTRDYIISPVKNDRGEFFCSEFSEGTMPKDADANGAYNIARK |
| | | GLWVLEQIRQKDEGEKVNLSMTNAEWLKYAQLHLL |
| SEQ | AacCas12b | MAVKSIKVKLRLDDMPEIRAGLWKLHKEVNAGVRYYTEWLSLLRQEN |
| ID | | LYRRSPNGDGEQECDKTAEECKAELLERLRARQVENGHRGPAGSDDEL |
| NO: | | LQLARQLYELLVPQAIGAKGDAQQIARKFLSPLADKDAVGGLGIAKAG |
| 27 | | NKPRWVRMREAGEPGWEEEKEKAETRKSADRTADVLRALADFGLKPL |
| | | MRVYTDSEMSSVEWKPLRKGQAVRTWDRDMFQQAIERMMSWESWN |
| | | QRVGQEYAKLVEQKNRFEQKNFVGQEHLVHLVNQLQQDMKEASPGL |
| | | ESKEQTAHYVTGRALRGSDKVFEKWGKLAPDAPFDLYDAEIKNVQRR |
| | | NTRRFGSHDLFAKLAEPEYQALWREDASFLTRYAVYNSILRKLNHAKM |
| | | FATFTLPDATAHPIWTRFDKLGGNLHQYTFLFNEFGERRHAIRFHKLLK |
| | | VENGVAREVDDVTVPISMSEQLDNLLPRDPNEPIALYFRDYGAEQHFTG |
| | | EFGGAKIQCRRDQLAHMHRRRGARDVYLNVSVRVQSQSEARGERRPP |
| | | YAAVFRLVGDNHRAFVHFDKLSDYLAEHPDDGKLGSEGLLSGLRVMS |
| | | VDLGLRTSASISVFRVARKDELKPNSKGRVPFFFPIKGNDNLVAVHERS |
| | | QLLKLPGETESKDLRAIREERQRTLRQLRTQLAYLRLLVRCGSEDVGRR |
| | | ERSWAKLIEQPVDAANHMTPDWREAFENELQKLKSLHGICSDKEWMD |
| | | AVYESVRRVWRHMGKQVRDWRKDVRSGERPKIRGYAKDVVGGNSIE |
| | | QIEYLERQYKFLKSWSFFGKVSGQVIRAEKGSRFAITLREHIDHAKEDRL |
| | | KKLADRIIMEALGYVYALDERGKGKWVAKYPPCQLILLEELSEYQFNN |
| | | DRPPSENNQLMQWSHRGVFQELINQAQVHDLLVGTMYAAFSSRFDAR |
| | | TGAPGIRCRRVPARCTQEHNPEPFPWWLNKFVVEHTLDACPLRADDLIP |
| | | TGEGEIFVSPFSAEEGDFHQIHADLNAAQNLQQRLWSDFDISQIRLRCD |
| | | WGEVDGELVLIPRLTGKRTADSYSNKVFYTNTGVTYYERERGKKRRK |
| | | VFAQEKLSEEEAELLVEADEAREKSVVLMRDPSGIINRGNWTRQKEFW |
| | | SMVNQRIEGYLVKQIRSRVPLQDSACENTGDI |
| SEQ | Cas12 | MKKIDNFVGCYPVSKTLRFKAIPIGKTQENIEKKRLVEEDEVRAKDYKA |
| ID | Variant | VKKLIDRYHREFIEGVLDNVKLDGLEEYYMLFNKSDREESDNKKIEIME |
| NO: | | ERFRRVISKSFKNNEEYKKIFSKKIIEEILPNYIKDEEEKELVKGFKGFYT |
| 28 | | AFVGYAQNRENMYSDEKKSTAISYRIVNENMPRFITNIKVFEKAKSILD |
| | | VDKINEINEYILNNDYYVDDFFNIDFFNYVLNQKGIDIYNAIIGGIVTGD |
| | | GRKIQGLNECINLYNQENKKIRLPQFKPLYKQILSESESMSFYIDEIESDD |
| | | MLIDMLKESLQIDSTINNAIDDLKVLFNNIFDYDLSGIFINNGLPITTISND |
| | | VYGQWSTISDGWNERYDVLSNAKDKESEKYFEKRRKEYKKVKSFSISD |
| | | LQELGGKDLSICKKINEIISEMIDDYKSKIEEIQYLFDIKELEKPLVTDLNK |
| | | IELIKNSLDGLKRIERYVIPFLGTGKEQNRDEVFYGYFIKCIDAIKEIDGV |
| | | YNKTRNYLTKKPYSKDKFKLYFENPQLMGGWDRNKESDYRSTLLRKN |
| | | GKYYVAIIDKSSSNCMMNIEEDENDNYEKINYKLLPGPNKMLPKVFFSK |
| | | KNREYFAPSKEIERIYSTGTFKKDTNFVKKDCENLITFYKDSLDRHEDW |
| | | SKSFDFSFKESSAYRDISEFYRDVEKQGYRVSFDLLSSNAVNTLVEEGK |
| | | LYLFQLYNKDFSEKSHGIPNLHTMYFRSLFDDNNKGNIRLNGGAEMFM |
| | | RRASLNKQDVTVHKANQPIKNKNLLNPKKTTTLPYDVYKDKRFTEDQ |
| | | YEVHIPITMNKVPNNPYKINHMVREQLVKDDNPYVIGIDRGERNLIYVV |
| | | VVDGQGHIVEQLSLNEIINENNGISIRTDYHTLLDAKERERDESRKQWK |
| | | QIENIKELKEGYISQVVHKICELVEKYDAVIALEDLNSGFKNSRVKVEK |
| | | QVYQKFEKMLITKLNYMVDKKKDYNKPGGVLNGYQLTTQFESFSKMG |
| | | TQNGIMFYIPAWLTSKMDPTTGFVDLLKPKYKNKADAQKFFSQFDSIR |
| | | YDNQEDAFVFKVNYTKFPRTDADYNKEWEIYTNGERIRVFRNPKKNNE |
| | | YDYETVNVSERMKELFDSYDLLYDKGELKETICEMEESKFFEELIKLFR |
| | | LTLQMRNSISGRTDVDYLISPVKNSNGYFYNSNDYKKEGAKYPKDADA |
| | | NGAYNIARKVLWAIEQFKMADEDKLDKTKISIKNQEWLEYAQTHCE |
| SEQ | Cas12 | MATLVSFTKQYQVQKTLRFELIPQGKTQANIDAKGFINDDLKRDENYM |
| ID | Variant | KVKGVIDELHKNFIEQTLVNVDYDWRSLATAIKNYRKDRSDTNKKNLE |
| NO: | | KTQEAARKEIIAWFEGKRGNSAFKNNQKSFYGKLFKKELFSEILRSDDL |
| 29 | | EYDEETQDAIACFDKFTTYFVGFHENRKNMYSTEAKSTSVAYRVVNEN |
| | | FSKFLSNCEAFSVLEAVCPNVLVEAEQELHLHKAFSDLKLSDVFKVEAY |
| | | NKYLSQTGIDYYNQIIGGISSAEGVRKIRGVNEVVNNAIQQNDELKVAL |
| | | RNKQFTMVQLFKQILSDRSTLSFVSEQFTSDQEVITVVKQFNDDIVNNK |
| | | VLAVVKTLFENFNSYDLEKIYINSKELASVSNALLKDWSKIRNAVLENK |
| | | IIELGANPPKTKISAVEKEVKNKDFSIAELASYNDKYLDKEGNDKEICSI |
| | | ANVVLEAVGALEIMLAESLPADLKTLENKNKVKGILDAYENLLHLLNY |
| | | FKVSAVNDVDLAFYGAFEKVYVDISGVMPLYNKVRNYATKKPYSVEK |
| | | FKLNFAMPTLADGWDKNKERDNGSIILLKDGQYYLGVMNPQNKPVID |
| | | NAVCNDAKGYQKMVYKMFPEISKMVTKCSTQLNAVKAHFEDNTNDF |
| | | VLDDTDKFISDLTITKEIYDLNNVLYDGKKKFQIDYLRNTGDFAGYHKA |
| | | LETWIDFVKEFLSKYRSTAIYDLTTLLPTNYYEKLDVFYSDVNNLCYKI |
| | | DYENISVEQVNEWVEEGNLYLFKIYNKDFATGSTGKPNLHTMYWNAV |
| | | FAEENLHDVVVKLNGGAELFYRPKSNMPKVEHRVGEKLVNRKNVNGE |
| | | PIADSVHKEIYAYANGKISKSELSENAQEELPLAIIKDVKHNITKDKRYL |
| | | SDKYFFHVPITLNYKANGNPSAFNTKVQAFLKNNPDVNIIGIDRGERNL |
| | | LYVVVIDQQGNIIDKKQVSYNKVNGYDYYEKLNQREKERIEARQSWG |
| | | AVGKIKELKEGYLSLVVREIADMMVKYNAIVVMENLNAGFKRVRGGI |
| | | AEKAVYQKFEKMLIDKLNYLVFKDVEAKEAGGVLNAYQLTDKFDSFE |
| | | KMGNQSGFLFYVPAAYTSKIDPVTGFANVFSTKHITNTEAKKEFICSFNS |
| | | LRYDEAKDKFVLECDLNKFKIVANSHIKNWKFIIGGKRIVYNSKNKTY |
| | | MEKYPCEDLKATLNASGIDFSSSEIINLLKNVPANREYGKLFDETYWAI |
| | | MNTLQMRNSNALTGEDYIISAVADDNEKVFDSRTCGAELPKDADANG |
| | | AYHIALKGLYLLQRIDISEEGEKVDLSIKNEEWFKFVQQKEYAR |
| SEQ | Cas12 | MKEQFINRYPLSKTLRFSLIPVGETENNFNKNLLLKKDKQRAENYEKVK |
| ID | Variant | CYIDRFHKEYIESVLSKARIEKVNEYANLYWKSNKDDSDIKAMESLEND |
| NO: | | MRKQISKQLTSTEIYKKRLFGKELICEDLPSFLTDKDERETVECFRSFTT |
| 30 | | YFKGFNTNRENMYSSDGKSTAIAYRCINDNLPRFLDNVKSFQKVFDNLS |
| | | DETITKLNTDLYNIFGRNIEDIFSVDYFEFVLTQSGIEIYNSMIGGYTCSD |
| | | KTKIQGLNECINLYNQQVAKNEKSKKLPLMKPLYKQILSEKDSVSFIPE |
| | | KFNSDNEVLHAIDDYYTGHIGDFDLLTELLQSLNTYNANGIFVKSGVAI |
| | | TDISNGAFNSWNVLRSAWNEKYEALHPVTSKTKIDKYIEKQDKIYKAIK |
| | | SFSLFELQSLGNENGNEITDWYISSINESNSKIKEAYLQAQKLLNSDYEK |
| | | SYNKRLYKNEKATELVKNLLDAIKEFQKLIKPLNGTGKEENKDELFYG |
| | | KFTSYYDSIADIDRLYDKVRNYITQKPYSKDKIKLNFDNPQLLGGWDK |
| | | NKESDYRTVLLHKDGLYYLAVMDKSHSKAFVDAPEITSDDKDYYEKM |
| | | EYKLLPGPNKMLPKVFFASKNIDTFQPSDRILDIRKRESFKKGATFNKAE |
| | | CHEFIDYFKDSIKKHDDWSQFGFKFSPTESYNDISEFYREISDQGYSVRF |
| | | NKISKNYIDGLVNNGYIYLFQIYNKDFSKYSKGTPNLHTLYFKMLFDER |
| | | NLSNVVYKLNGEAEMFYREASIGDKEKITHYANQPIKNKNPDNEKKES |
| | | VFEYDIVKDKRFTKRQFSLHLPITINFKAHGQEFLNYDVRKAVKYKDD |
| | | NYVIGIDRGERNLIYISVINSNGEIVEQMSLNEIISDNGHKVDYQKLLDT |
| | | KEKERDKARKNWTSVENIKELKEGYISQVVHKICELVIKYDAVIAMED |
| | | LNFGFKRGRFPVEKQVYQKFENMLISKLNLLIDKKAEPTEDGGLLRAY |
| | | QLTNKFDGVNKAKQNGIIFYVPAWDTSKIDPATGFVNLLKPKCNTSVPE |
| | | AKKLFETIDDIKYNANTDMFEFYIDYSKFPRCNSDFKKSWTVCTNSSRIL |
| | | TFRNKEKNNKWDNKQIVLTDEFKSLFNEFGIDYKGNLKDSILSISNADF |
| | | YRRLIKLLSLTLQMRNSITGSTLPEDDYLISPVANKSGEFYDSRNYKGTN |
| | | AALPCDADANGAYNIARKALWAINVLKDTPDDMLNKAKLSITNAEWL |
| | | EYTQK |
| SEQ | Cas12 | MNNPRGAFGGFTNLYSLSKTLRFELKPYLEIPEGEKGKLFGDDKEYYK |
| ID | Variant | NCKTYTEYYLKKANKEYYDNEKVKNTDLQLVNFLHDERIEDAYQVLK |
| NO: | | PVFDTLHEEFITDSLESAEAKKIDFGNYYGLYEKQKSEQNKDEKKKIDK |
| 31 | | PLETERGKLRKAFTPIYEAEGKNLKNKAGKEKKDKDILKESGFKVLIEA |
| | | GILKYIKNNIDEFADKKLKNNEGKEITKKDIETALGAENIEGIFDGFFTYF |
| | | SGFNQNRENYYSTEEKATAVASRIVDENLSKFCDNILLYRKNENDYLKI |
| | | FNFLKNKGKDLKLKNSKFGKENEPEFIPAYDMKNDEKSFSVADFVNCL |
| | | SQGEIEKYNAKIANANYLINLYNQNKDGNSSKLSMFKILYKQIGCGEKK |
| | | DFIKTIKDNAELKQILEKACEAGKKYFIRGKSEDGGVSNIFDFTDYIQSH |
| | | ENYKGVYWSDKAINTISGKYFANWDTLKNKLGDAKVFNKNTGEDKA |
| | | DVKYKVPQAVMLSELFAVLDDNAGEDWREKGIFFKASLFEGDQNKSEI |
| | | IKNANRPSQALLKMICDDMESLAKNFIDSGDKILKISDRDYQKDENKQK |
| | | IKNWLDNALWINQILKYFKVKANKIKGDSIDARIDSGLDMLVFSSDNPA |
| | | EDYDMIRNYLTQKPQDEINKLKLNFENSSLAGGWDENKEKDNSCIILKD |
| | | EQDKQYLAVMKYENTKVFEQKNSQLYIADNAAWKKMIYKLVPGASK |
| | | TLPKVFFSKKWTANRPTPSDIVEIYQKGSFKKENVDFNDKKEKDESRKE |
| | | KNREKIIAELQKTCWMDIRYNIDGKIESAKYVNKEKLAKLIDFYKENLK |
| | | KYPSEEESWDRLFAFGFSDTKSYKSIDQFYIEVDKQGYKLEFVTINKAR |
| | | LDEYVRDGKIYLFEIRSRDNNLVNGEEKTSAKNLQTIYWNAAFGGDDN |
| | | KPKLNGEAEIFYRPAIAENKLNKKKDKNGKEIIDGYRFSKEKFIFHCPITL |
| | | NFCLKETKINDKLNAALAKPENGQGVYFLGIDRGEKHLAYYSLVNQKG |
| | | EILEQGTLNLPFLDKNGKSRSIKVEKKSFEKDSNGGIIKDKDGNDKIKIEF |
| | | VECWNYNDLLDARAGDRDYARKNWTTIGTIKELKDGYISQVVRKIVD |
| | | LSIYKNTETKEFREMPAFIVLEDLNIGFKRGRQKIEKQVYQKLELALAK |
| | | KLNFLVDKKADIGEIGSVTKAIQLTPPVNNFGDMENRKQFGNMLYIRA |
| | | DYTSQTDPATGWRKSIYLKSGSESNVKEQIEKSFFDIRYESGDYCFEYR |
| | | DRHGKMWQLYSSKNGVSLDRFHGERNNSKNVWESEKQPLNEMLDILF |
| | | DEKRFDKSKSLYEQMFKGGVALTRLPKEINKKDKPAWESLRFVIILIQQI |
| | | RNTGKNGDDRNGDFIQSPVRDEKTGEHFDSRIYLDKEQKGEKADLPTS |
| | | GDANGAYNIARKGIVVAEHIKRGFDKLYISDEEWDTWLAGDEIWDKW |
| | | LKENRESLTKTRK |
| SEQ | Cas12 | MNGNRIIVYREFVGVTPVAKTLRNELRPIGHTQEHIIHNGLIQEDELRQE |
| ID | Variant | KSTELKNIMDDYYREYIDKSLSGVTDLDFTLLFELMNLVQSSPSKDNKK |
| NO: | | ALEKEQSKMREQICTHMQSDSNYKNIFNAKFLKEILPDFIKNYNQYDAK |
| 32 | | DKAGKLETLALFNGFSTYFTDFFEKRKNVFTKEAVSTSIAYRIVHENSLT |
| | | FLANMTSYKKISEKALDEIEVIEKNNQDKMGDWELNQIFNPDFYNMVLI |
| | | QSGIDFYNEICGVVNAHMNLYCQQTKNNYNLFKMRKLHKQILAYTSTS |
| | | FEVPKMFEDDMSVYNAVNAFIDETEKGNIIGKLKDIVNKYDELDEKRIY |
| | | ISKDFYETLSCFMSGNWNLITGCVENFYDENIHAKGKSKEEKVKKAVK |
| | | EDKYKSINDVNDLVEKYIDEKERNEFKNSNAKQYIREISNIITDTETAHL |
| | | EYDEHISLIESEEKADEMKKRLDMYMNMYHWAKAFIVDEVLDRDEMF |
| | | YSDIDDIYNILENIVPLYNRVRNYVTQKPYNSKKIKLNFQSPTLANGWS |
| | | QSKEFDNNAIILIRDNKYYLAIFNAKNKPDKKIIQGNSDKKNDNDYKKM |
| | | VYNLLPGANKMLPKVFLSKKGIETFKPSDYIISGYNAHKHIKTSENFDIS |
| | | FCRDLIDYFKNSIEKHAEWRKYEFKFSATDSYNDISEFYREVEMQGYRI |
| | | DWTYISEADINKLDEEGKIYLFQIYNKDFAENSTGKENLHTMYFKNIFSE |
| | | ENLKDIIIKLNGQAELFYRRASVKNPVKHKKDSVLVNKTYKNQLDNGD |
| | | VVRIPIPDDIYNEIYKMYNGYIKENDLSEAAKEYLDKVEVRTAQKDIVK |
| | | DYRYTVDKYFIHTPITINYKVTARNNVNDMAVKYIAQNDDIHVIGIDRG |
| | | ERNLIYISVIDSHGNIVKQKSYNILNNYDYKKKLVEKEKTREYARKNW |
| | | KSIGNIKELKEGYISGVVHEIAMLMVEYNAIIAMEDLNYGFKRGRFKVE |
| | | RQVYQKFESMLINKLNYFASKGKSVDEPGGLLKGYQLTYVPDNIKNLG |
| | | KQCGVIFYVPAAFTSKIDPSTGFISAFNFKSISTNASRKQFFMQFDEIRYC |
| | | AEKDMFSFGFDYNNFDTYNITMSKTQWTVYTNGERLQSEFNNARRTG |
| | | KTKSINLTETIKLLLEDNEINYADGHDVRIDMEKMDEDKNSEFFAQLLS |
| | | LYKLTVQMRNSYTEAEEQEKGISYDKIISPVINDEGEFFDSDNYKESDD |
| | | KECKMPKDADANGAYCIALKGLYEVLKIKSEWTEDGFDRNCLKLPHA |
| | | EWLDFIQNKRYE |
| SEQ | Cas12 | MKKIDSFVNYYPLSKTLRFSLIPVGKTEDNFNAKLLLEEDEKRAIEYEK |
| ID | Variant | VKRYIDRYHKHFIETVLANFHLDDVNEYAELYYKAGKDDKDLKYMEK |
| NO: | | LEGKMRKSISAAFTKDKKYKEIFGQEIIKNILPEFLENEDEKESVKMFQG |
| 33 | | FFTYFTGFNDNRKNMYTHEAQTTAISYRCINENLPKFLDNVQSFAKIKE |
| | | SISSDIMNKLDEVCMDLYGVYAQDMFCTDYFSFVLSQSGIDRYNNIIGG |
| | | YVDDKGVKIQGINEYINLYNQQVDEKNKRLPLMKKLYKQILIEKESISFI |
| | | PEKFESDNIVINAISDYYHNNVENLFDDFNKLFNEFSEYDDNGIFVTSGL |
| | | AVTDISNAVFGSWNIISDSWNEEYKDSHPMKKTTNAEKYYEDMKKEY |
| | | KKNLSFTIAELQRLGEAGCNDECKGDIKEYYKTTVAEKIENIKNAYEISK |
| | | DLLASDYEKSNDKKLCKNDSAISLLKNLLDSIKDLEKTIKPLLGTGKEE |
| | | NKDDVFYGKFTNLYEMISEIDRLYDKVRNYVTQKPYSKDKIKLNFENP |
| | | QHLGGWDKNKERDYRSVLLKKEDKYYLAIMDKSNNKAFIDFPDDGEC |
| | | YEKIEYKLLPGPNKMLPKVFFASSNIEYFAPSKKILEIRSRESFKKGDMF |
| | | NLKDCHEFIDFFKESIKKHEDWSQFGFEFSPTEKYNDISEFYNEVKIQGY |
| | | SLKYKNVSKKYIDELIECGQLYLFQIYNKDFSVYAKGNPNLHTMYFKM |
| | | LFDERNLANVVYQLNGGAEMFYRKASIKDSEKIVHHANQPIKNKNADN |
| | | VKKESVFEYDIIKDKRFTKRQFSIHIPITLNFKAKGQNFINNDVRMALKK |
| | | ADENYVIGIDRGERNLLYICVINSKGEIVEQKSLNEIIGDNGYRVDYHKL |
| | | LDKKEAERDEARKSWGTIENIKELKEGYLSQIVHEISKLVIKYDAVIAIE |
| | | DLNSGFKKGRFKVEKQVYQKFENMLCTKLNYLVDKNADANECGGLL |
| | | KAYQLINKEDGANRGRQNGIIFSVPAWLTSKIDPVTGFADLLRPKYKSV |
| | | SESVEFISKIDNIRYNSKEDYFEFDIDYSKFPNSTASYKKKWTVCTYGERI |
| | | INVRNKEKNNMWDNKTIVLTDEFKKLFADFGVDVSKNIKESVLAIDSK |
| | | DFYYRFINLLANTLQLRNSEVGNVDVDYLISPVKGVDGSFYDSRLVKE |
| | | KTLPENADANGAYNIARKALWAIDVLKQTKDEELKNANLSIKNAEWL |
| | | EYVQK |
| SEQ | Cas12 | MRTMVTFEDFTKQYQVSKTLRFELIPQGKTLENMKRDGIISVDRQRNE |
| ID | Variant | DYQKAKGILDKLYKYILDFTMETVVIDWEALATATEEFRKSKDKKTYE |
| NO: | | KVQSKIRTALLEHVKKQKVGTEDLFKGMFSSKIITGEVLAAFPEIRLSDE |
| 34 | | ENLILEKFKDFTTYFTGFFENRKNVFTDEALSTSFTYRLVNDNFIKFFDN |
| | | CIVFKNVVNISPHMAKSLETCASDLGIFPGVSLEEVFSISFYNRLLTQTGI |
| | | DQFNQLLGGISGKEGEHKQQGLNEIINLAMQQNLEVKEVLKNKAHRFT |
| | | PLFKQILSDRSTMSFIPDAFADDDEVLSAVDAYRKYLSEKNIGDRAFQLI |
| | | SDMEAYSPELMRIGGKYVSVLSQLLFYSWSEIRDGVKAYKESLITGKKT |
| | | KKELENIDKEIKYGVTLQEIKEALPKKDIYEEVKKYAMSVVKDYHAGL |
| | | AEPLPEKIETDDERASIKHIMDSMLGLYRFLEYFSHDSIEDTDPVFGECL |
| | | DTILDDMNETVPLYNKVRNFSTRKVYSTEKFKLNFNNSSLANGWDKN |
| | | KEQANGAILLRKEGEYFLGIFNSKNKPKLVSDGGAGIGYEKMIYKQFPD |
| | | FKKMLPKCTISLKDTKAHFQKSDEDFTLQTDKFEKSIVITKQIYDLGTQT |
| | | VNGKKKFQVDYPRLTGDMEGYRAALKEWIDFGKEFIQAYTSTAIYDTS |
| | | LFRDSSDYPDLPSFYKDVDNICYKLTFEWIPDAVIDDCIDDGSLYLFKLH |
| | | NKDFSSGSIGKPNLHTLYWKALFEEENLSDVVVKLNGQAELFYRPKSLT |
| | | RPVVHEEGEVIINKTTSTGLPVPDDVYVELSKFVRNGKKGNLTDKAKN |
| | | WLDKVTVRKMPHAITKDRRFTVDKFFFHVPITLNYKADSSPYRFNDFV |
| | | RQYIKDCSDVKIIGIDRGERNLIYAVVIDGKGNIIEQRSFNTVGTYNYQE |
| | | KLEQKEKERQTARQDWATVTKIKDLKKGYLSAVVHELSKMIVKYKAI |
| | | VALENLNVGFKRMRGGIAERSVYQQFEKALIDKLNYLVFKDEEQSGYG |
| | | GVLNAYQLTDKFESFSKMGQQTGFLFYVPAAYTSKIDPLTGFINPFSWK |
| | | HVKNREDRRNFLNLFSKLYYDVNTHDFVLAYHHSNKDSKYTIKGNWEI |
| | | ADWDILIQENKEVFGKTGTPYCVGKRIVYMDDSTTGHNRMCAYYPHT |
| | | ELKKLLSEYGIEYTSGQDLLKIIQEFDDDKLVKGLFYIIKAALQMRNSNS |
| | | ETGEDYISSPIEGRPGICFDSRAEADTLPYDADANGAFHIAMKGLLLTER |
| | | IRNDDKLAISNEEWLNYIQEMRG |
| SEQ | Cas12 | MNKDIRKNFTDFVGISEIQKTLRFILIPIGKTAQNIDKYNMFEDDEIRHEY |
| ID | Variant | YPILKEACDDFYRNHIDQQFENLELDWSKLDEALASEDRDLINETRATY |
| NO: | | RQVLFNRLKNSVDIKGDSKKNKTLSLESSDKNLGKKKTKNTFQYNFND |
| 35 | | LFKAKLIKAILPLYIEYIYEGEKLENAKKALKMYNRFTSRLSNFWQARA |
| | | NIFTDDEISTGSPYRLVNDNFTIFRINNSIYTKNKPFIEEDILEFEKKLKSK |
| | | KIIKDFESVDDYFTVNAFNKLCTQNGIDKYNSILGGFTTKEREKVKGLN |
| | | ELFNLAQQSINKGKKGEYRKNIRLGKLTKLKKQILAISDSTSFLIEQIEDD |
| | | QDLYNKIKDFFELLLKEEIENENIFTQYANLQKLIEQADLSKIYINAKHL |
| | | NKISHQVTGKWDSLNKGIALLLENININEESKEKSEVISNGQTKDISSEA |
| | | YKRYLQIQSEEKDIERLRTQIYFSLEDLEKALDLVLIDENMDRSDKSILS |
| | | YVQSPDLNVNFERDLTDLYSRIMKLEENNEKLLANHSAIDLIKEFLDLI |
| | | MLRYSRWQILFCDSNYELDQTFYPIYDAVMEILSNIIRLYNLARNYLSR |
| | | KPDRMKKKKINFNNPTLADGWSESKIPDNSSMLFIKDGMYYLGIIKNRA |
| | | AYSELLEAESLQSSEKKKSENSSYERMNYHFLPDAFRSIPKSSIAMKAV |
| | | KEHFEINQKTADLLLDTDKFSKPLRITKEIFDMQYVDLHKNKKKYQVD |
| | | YLRDTGDKKGYRKALNTWLNFCKDFISKYKGRNLFDYSKIKDADHYE |
| | | TVNEFYNDVDKYSYHIFFTSVAETTVEKFISEGKLYLFQLYNKDFSPHST |
| | | GKPNLHTIYWRALFSEENLTSKNIKLNGQAEIFFRPKQIETPFTHKKGSIL |
| | | VNRFDVNGNPIPINVYQEIKGFKNNVIKWDDLNKTTQEGLENDQYLYF |
| | | ESEFEIIKDRRYTEDQLFFHVPISFNWDIGSNPKINDLATQYIVNSNDIHII |
| | | GIDRGENHLIYYSVIDLQGAIVEQGSLNTITEYTENKFLNNKTNNLRKIP |
| | | YKDILQQREDERADARIKWHAIDKIKDLKDGYLGQIVHFLAKLIIKYNA |
| | | IVILEDLNYGFKRGRFKVERQVYQKFEMALMKKLNVLVFKDYDIDEIG |
| | | GPLKPWQLTRPIDSYERMGRQNGILFYVPAAYTSAVDPVTGFANLFYL |
| | | NNVKNSEKFHFFSKFESIKYHSDQDMFSFAFDYNNFGTTTRINDLSKSK |
| | | WQVFTNHERSVWNNKEKNYVTQNLTDLIKKLLRTYNIEFKNNQNVLD |
| | | SILKIENNTDKENFARELFRLFRLTIQLRNTTVNENNTEITENELDYIISPV |
| | | KDKNGNFFDSRDELKNLPDNGDANGAYNIARKGLLYIEQLQESIKTGK |
| | | LPTLSISTLDWFNYIMK |
| SEQ | Cas12 | MTPIFCNFVVYQIMLFNNNININVKTMNKKHLSDFTNLFPVSKTLRFRL |
| ID | Variant | EPQGKTMENIVKAQTIETDEERSHDYEKTKEYIDDYHRQFIDDTLDKFA |
| NO: | | FKVESTGNNDSLQDYLDAYLSANDNRTKQTEEIQTNLRKAIVSAFKMQ |
| 36 | | PQFNLLFKKEMVKHLLPQFVDTDDKKRIVAKFNDFTTYFTGFFTNREN |
| | | MYSDEAKSTSIAYRIVNQNLIKFVENMLTFKSHILPILPQEQLATLYDDF |
| | | KEYLNVASIAEMFELDHFSIVLTQRQIEVYNSVIGGRKDENNKQIKPGL |
| | | NQYINQHNQAVKDKSARLPLLKPLFNQILSEKAGVSFLPKQFKSASEVV |
| | | KSLNEAYAELSPVLAAIQDVVTNITDYDCNGIFIKNDLGLTDIAQRFYG |
| | | NYDAVKRGLRNQYELETPMHNGQKAEKYEEQVAKHLKSIESVSLAQIN |
| | | QVVTDGGDICDYFKAFGATDDGDIQRENLLASINNAHTAISPVLNKENA |
| | | NDNELRKNTMLIKDLLDAIKRLQWFAKPLLGAGDETNKDQVFYGKFEP |
| | | LYNQLDETISPLYDKVRSYLTKKPYSLDKFKINFEKSNLLGGWDPGADR |
| | | KYQYNAVILRKDNDFYLGIMRDEATSKRKCIQVLDCNDEGLDENFEKV |
| | | EYKQIKPSQNMPRCAFAKKECEENADIMELKRKKNAKSYNTNKDDKN |
| | | ALIRHYQRYLDRTYPEFGFVYKDADEYDTVKAFTDSMDSQDYKLSFLQ |
| | | VSETGLNKLVDEGDLYLFKITNKDFSSYAKGRPNLHTIYWRMLFDPKN |
| | | LANVVYKLEGKAEVFFRRKSLASTTTHKAKQAIKNKSRYNEAVKPQST |
| | | FDYDIIKDRRFTADKFEFHVPIKMNFKAAGWNSTRLTNEVREFIKSQGV |
| | | RHIIGIDRGERHLLYLTMIDMDGNIVKQCSLNAPAQDNARASEVDYHQ |
| | | LLDSKEADRLAARRNWGTIENIKELKQGYLSQVVHLLATMMVDNDAI |
| | | LVLENLNAGFMRGRQKVEKSVYQKFEKMLIDKLNYIVDKGQSPDKPT |
| | | GALHAVQLTGLYSDFNKSNMKRANVRQCGFVFYIPAWNTSKIDPVTGF |
| | | VNLFDTHLSSMGEIKAFFSKFDSIRYNQDKGWFEFKFDYSRFTTRAEGC |
| | | RTQWTVCTYGERIWTHRSKNQNNQFVNDTVNVTQQMLQLLQDCGIDP |
| | | NGNLKEAIANIDSKKSLETLLHLFKLTVQMRNSVTGSEVDYMISPVADE |
| | | RGHFFDSRESDEHLPANADANGAFNIARKGLMVVRQIMATDDVSKIKF |
| | | AVSNKDWLRFAQHIDD |
| SEQ | Cas12 | MNKGGYVIMEKMTEKNRWENQFRITKTIKEEIIPTGYTKVNLQRVNML |
| ID | Variant | KREMERNEDLKKMKEICDEYYRNMIDVSLRLEQVRTLGWESLIHKYR |
| NO: | | MLNKDEKEIKALEKEQEDLRKKISKGFGEKKAWTGEQFIKKILPQYLM |
| 37 | | DHYTGEELEEKLRIVKKFKGCTMFLSTFFKNRENIFTDKPIHTAVGHRIT |
| | | SENAMLFAANINTYEKMESNVTLEIERLQREFWRRGINISEIFTDAYYVN |
| | | VLTQKQIEAYNKICGDINQHMNEYCQKQKLKFSEFRMRELKKQILAVV |
| | | GEHFEIPEKIESTKEVYRELNEYYESLKELHGQFEELKSVQLKYSQIYVQ |
| | | KKGYDRISRYIGGQWDLIQECMKKDCASGMKGTKKNHDAKIEEEVAK |
| | | VKYQSIEHIQKLVCTYEEDRGHKVTDYVDEFIVSVCDLLGADHIITRDG |
| | | ERIELPLQYEPGTDLLKNDTINQRRLSDIKTILDWHMDMLEWLKTFLVN |
| | | DLVIKDEEFYMAIEELNERMQCVISVYNRIRNYVTQKGYEPEKIRICFDK |
| | | GTILTGWTTGDNYQYSGFLLMRNDKYYLGIINTNEKSVRKILDGNEEC |
| | | KDENDYIRVGYHLINDASKQLPRIFVMPKAGKKSEILMKDEQCDYIWD |
| | | GYCHNKHNESKEFMRELIDYYKRSIMNYDKWEGYCFKFSSTESYDNM |
| | | QDFYKEVREQSYNISFSYINENVLEQLDKDGKIYLFQVYNKDFAAGSTG |
| | | TPNLHTMYLQNLFSSQNLELKRLRLGGNAELFYRPGTEKDVTHRKGSIL |
| | | VDRTYVREEKDGIEVRDTVPEKEYLEIYRYLNGKQKGDLSESAKQWLD |
| | | KVHYREAPCDIIKDKRYAQEKYFLHFSVEINPNAEGQTALNDNVRRWL |
| | | SEEEDIHVIGIDRGERNLIYVSLMDGKGRIKDQKSYNIVNSGNKEPVDYL |
| | | AKLKVREKERDEARRNWKAIGKIKDIKTGYLSYVVHEIVEMAVREKAII |
| | | VMEDLNYGFKRGRFKVERQVYQKFEEMLINKLNYVVDKQLSVDEPGG |
| | | LLRGYQLAFIPKDKKSSMRQNGIVFYVPAGYTSKIDPTTGFVNIFKFPQF |
| | | GKGDDDGNGKDYDKIRAFFGKFDEIRYECDEKVTADNTREVKERYRF |
| | | DFDYSKFETHLVHMKKTKWTVYAEGERIKRKKVGNYWTSEVISDIALR |
| | | MSNTLNIAGIEYKDGHNLVNEICALRGKQAGIILNELLEIVRLTVQLRNS |
| | | TTEGDVDERDEIISPVLNEKYGCFYHSTEYKQQNGDVLPKDADANGAY |
| | | CIGLKGIYEIRQIKNKWKEDMTKGEGKALNEGMRISHDQWFEFIQNMN |
| | | KGE |
| SEQ | Cas12 | MNELVKNRCKQTKTICQKLIPIGKTRETIEKYNLMEIDRKIAANKELMN |
| ID | Variant | KLFSLIAGKHINDTLSKCTDLDFEPLLTSLSSLNNAKENDRDNLREYYDS |
| NO: | | VFEEKKTLAEEISSRLTAVKFAGKDFFTKNIPDFLETYEGDDKNEMSEL |
| 38 | | VSLVIENTVTAGYVKKLEKIDRSMEYRLVSGTVVKRVLTDNADIYEKNI |
| | | EKAKDFDYGVLNIDEASQFTTLVAKDYANYLTADGIAIYNVGIGKINLA |
| | | LNEYCQKNKEYSYNKLALLPLQKMLYGEKLSLFEKLEDFTSDEELINSY |
| | | NKFAKTVNESGLAEIIKKAVPSYDEIVIKPNKISNYSNSITGHWSLVNRI |
| | | MKDYLENNGIKNADKYMEKGLTLSEIGDALENKNIKHSDFISNLINDLG |
| | | HTYTEIKENKESLKKDESVNALIIKKELDMLLSILQNLKVFDIDNEMFDT |
| | | GFGIEVSKAIEILGYGVPLYNKIRNYITKKPDPKKKFMTKFGSATIGTGIT |
| | | TSVEGSKKATFLKDGDAVFLLLYNTAGCKANNVSVSNLADLINSSLEIE |
| | | NSGKCYQKMIYQTPGDIKKQIPRVFVYKSEDDDLIKDFKAGLHKTDLSF |
| | | LNGRLIPYLKEAFATHETYKNYTFSYRNSYESYDEFCEHMSEQAYILEW |
| | | KWIDKKLIDDLVEDGSLLMFRVWNRFMKKKEGKISKHAKIVNELFSDE |
| | | NASNAAIKLLSVFDIFYRDKQIDNPIVHKAGTTLYNKRTKDGEVIVDYT |
| | | TMVKNKEKRPNVYTTTKKYDIIKDRRYTEEQFEIHLHVNIGKEENKEKL |
| | | ETSKVINEKKNTLVVTRSNEHLLYVVIFDENDNILLKKSLNTVKGMNFK |
| | | SKLEVVEIQKKENMQSWKTVGSNQALMEGYLSFAIKEIADLVKEYDAI |
| | | LVLEQNSVGKNILNERVYTRFKEMLITNLSLDVDYENKDFYSYTELGG |
| | | KVASWRDCVTNGICIQVPSAYKYKDPTTSFSTISMYAKTTAEKSKKLKQ |
| | | IKSFKYNRERGLFELVIAKGVGLENNIVCDSFGSRSIIENDISKEVSCTLKI |
| | | EKYLIDAGIEYNDEKEVLKDLDTAAKTDAVHKAVTLLLKCFNESPDGR |
| | | YYISPCGEHFTLCDAPEVLSAINYYIRSRYIREQIVEGVKKMEYKKTILL |
| | | AK |
| SEQ | Cas12 | MNYKTGLEDFIGKESLSKTLRNALIPTESTKIHMEEMGVIRDDELRAEK |
| ID | Variant | QQELKEIMDDYYRTFIEEKLGQIQGIQWNSLFQKMEETMEDISVRKDLD |
| NO: | | KIQNEKRKEICCYFTSDKRFKDLFNAKLITDILPNFIKDNKEYTEEEKAE |
| 39 | | KEQTRVLFQRFATAFTNYFNQRRNNFSEDNISTAISFRIVNENSEIHLQN |
| | | MRAFQRIEQQYPEEVCGMEEEYKDMLQEWQMKHIYSVDFYDRELTQP |
| | | GIEYYNGICGKINEHMNQFCQKNRINKNDFRMKKLHKQILCKKSSYYEI |
| | | PFRFESDQEVYDALNEFIKTMKKKEIIRRCVHLGQECDDYDLGKIYISSN |
| | | KYEQISNALYGSWDTIRKCIKEEYMDALPGKGEKKEEKAEAAAKKEEY |
| | | RSIADIDKIISLYGSEMDRTISAKKCITEICDMAGQISIDPLVCNSDIKLLQ |
| | | NKEKTTEIKTILDSFLHVYQWGQTFIVSDIIEKDSYFYSELEDVLEDFEGI |
| | | TTLYNHVRSYVTQKPYSTVKFKLHFGSPTLANGWSQSKEYDNNAILLM |
| | | RDQKFYLGIFNVRNKPDKQIIKGHEKEEKGDYKKMIYNLLPGPSKMLP |
| | | KVFITSRSGQETYKPSKHILDGYNEKRHIKSSPKFDLGYCWDLIDYYKE |
| | | CIHKHPDWKNYDFHFSDTKDYEDISGFYREVEMQGYQIKWTYISADEI |
| | | QKLDEKGQIFLFQIYNKDFSVHSTGKDNLHTMYLKNLFSEENLKDIVLK |
| | | LNGEAELFFRKASIKTPIVHKKGSVLVNRSYTQTVGNKEIRVSIPEEYYT |
| | | EIYNYLNHIGKGKLSSEAQRYLDEGKIKSFTATKDIVKNYRYCCDHYFL |
| | | HLPITINFKAKSDVAVNERTLAYIAKKEDIHIIGIDRGERNLLYISVVDVH |
| | | GNIREQRSFNIVNGYDYQQKLKDREKSRDAARKNWEEIEKIKELKEGY |
| | | LSMVIHYIAQLVVKYNAVVAMEDLNYGFKTGRFKVERQVYQKFETML |
| | | IEKLHYLVFKDREVCEEGGVLRGYQLTYIPESLKKVGKQCGFIFYVPAG |
| | | YTSKIDPTTGFVNLFSFKNLTNRESRQDFVGKFDEIRYDRDKKMFEFSF |
| | | DYNNYIKKGTILASTKWKVYTNGTRLKKIVVNGKYTSQSMEVELTDA |
| | | MEKMLQRAGIEYHDGKDLKGQIVEKGIEAEIIDIFRLTVQMRNSRSESE |
| | | DREYDRLISPVLNDKGEFFDTATADKTLPQDADANGAYCIALKGLYEV |
| | | KQIKENWKENEQFPRNKLVQDNKTWFDFMQKKRYL |
| SEQ | Cas12 | MEDKQFLERYKEFIGLNSLSKTLRNSLIPVGSTLKHIQEYGILEEDSLRA |
| ID | Variant | QKREELKGIMDDYYRNYIEMHLRDVHDIDWNELFEALTEVKKNQTDD |
| NO: | | AKKRLEKIQEKKRKEIYQYLSDDAVFSEMFKEKMISGILPDFIRCNEGYS |
| 40 | | EEEKEEKLKTVALFHRFTSSFNDFFLNRKNVFTKEAIVTAIGYRVVHEN |
| | | AEIFLENMVAFQNIQKSAESQISIIERKNEHYFMEWKLSHIFTADYYMM |
| | | LMTQKAIEHYNEMCGVVNQQMREYCQKEKKNWNLYRMKRLHKQILS |
| | | NASTSFKIPEKYENDAEVYESVNSFLQNVMEKTVMERIAVLKNSTDNF |
| | | DLSKIYITAPYYEKISNYLCGSWNTITDCLTHYYEQQIAGKGARKDQKV |
| | | KAAVKADKWKSLSEIEQLLKEYARAEEVKRKPEEYIAEIENIVSLKEAH |
| | | LLEYHPEVNLIENEKYATEIKDVLDNYMELFHWMKWFYIEEAVEKEVN |
| | | FYGELDDLYEEIKDIVPLYNKVRNYVTQKPYSDTKIKLNFGTPTLANGW |
| | | SKSKEYDYNAILLQKDGKYYMGIFNPIQKPEKEIIEGHSQPLEGNEYKK |
| | | MVYYYLPSANKMLPKVLLSKKGMEIYQPSEYIINGYKERRHIKSEEKFD |
| | | LQFCHDLIDYFKSGIERNSDWKVFGFDFSDTDTYQDISGFYREVEDQGY |
| | | KIDWTYIKEADIDRLNEEGKLYLFQIYNKDFSEKSTGRENLHTMYLKNL |
| | | FSEENVREQVLKLNGEAEIFFRKSSVKKPIIHKKGTMLVNRTYMEEVNG |
| | | NSVRRNIPEKEYQEIYNYKNHRLKGELSTEAKKYLEKAVCHETKKDIV |
| | | KDYRYSVDKFFIHLPITINYRASGKETLNSVAQRYIAHQNDMHVIGIDR |
| | | GERNLIYVSVINMQGEIKEQKSFNIINEFNYKEKLKEREQSRGAARRNW |
| | | KEIGQIKDLKEGYLSGVIHEIAKMMIKYHAIIAMEDLNYGFKRGRFKVE |
| | | RQVYQKFENMLIQKLNYLVFKDRPADEDGGVLRGYQLAYIPDSVKKM |
| | | GRQCGMIFYVPAAFTSKIDPTTGFVDIFKHKVYTTEQAKREFILSFDEIC |
| | | YDVERQLFRFTFDYANFVTQNVTLARNNWTIYTNGTRAQKEFGNGRM |
| | | RDKEDYNPKDKMVELLESEGIEFKSGKNLLPALKKVSNAKVFEELQKI |
| | | VRFTVQLRNSKSEENDVDYDHVISPVLNEEGNFFDSSKYKNKEEKKESL |
| | | LPVDADANGAYCIALKGLYIMQAIQKNWSEEKALSPDVLRLNNNDWF |
| | | DYIQNKRYR |
| SEQ | Cas12 | MEKSLNDFIGLYSVSKTLRFELKPVSETLENIKKFHFLEEDKKKANDYK |
| ID | Variant | DVKKIIDNYHKYFIDDVLKNASFNWKKLEEAIREYNKNKSDDSALVAE |
| NO: | | QKKLGDAILKLFTSDKRYKALTAATPKELFESILPDWFGEQCNQDLNK |
| 41 | | AALKTFQKFTSYFTGFQENRKNVYSAEAIPTAVPYRIVNDNFPKFLQNV |
| | | LIFKTIQEKCPQIIDEVEKELSSYLGKEKLAGIFTLESFNKYLGQGGKENQ |
| | | RGIDFYNQIIGGVVEKEGGINLRGVNQFLNLYWQQHPDFTKEDRRIKM |
| | | VPLYKQILSDRSSLSFKIESIENDEELKNALLECADKLELKNDEKKSIFEE |
| | | VCDLFSSVKNLDLSGIYINRKDINSVSRILTGDWSWLQSRMNVYAEEKF |
| | | TTKAEKARWQKSLDDEGENKSKGFYSLTDLNEVLEYSSENVAETDIRIT |
| | | DYFEHRCRYYVDKETEMFVQGSELVALSLQEMCDDILKKRKAMNTVL |
| | | ENLSSENKLREKTDDVAVIKEYLDAVQELLHRIKPLKVNGVGDSTFYSV |
| | | YDSIYSALSEVISVYNKTRNYITKKAASPEKYKLNFDNPTLADGWDLNK |
| | | EQANTSVILRKDGMFYLGIMNPKNKPKFAEKYDCGNESCYEKMIYKQF |
| | | DATKQIPKCSTQKKEVQKYFLSGATEPYILNDKKSFKSELIITKDIWFMN |
| | | NHVWDGEKFVPKRDNETRPKKFQIGYFKQTGDFDGYKNALSNWISFC |
| | | KNFLQSYLSATVYDYNFKNSEEYEGLDEFYNYLNATCYKLNFINIPETEI |
| | | NKMVSEGKLYLFQIYNKDFASGSTGMPNMHTLYWKNLFSDENLKNVC |
| | | LKLNGEAELFYRPAGIKEPVIHKEGSYLVNRTTEDGESIPEKIYFEIYKNA |
| | | NGKLEKLSDEAQNYISNHEVVIKKAGHEIIKDRHYTEPKFLFHVPLTINF |
| | | KASGNSYSINENVRKFLKNNPDVNIIGLDRGERHLIYLSLINQKGEIIKQF |
| | | TFNEVERNKNGRTIKVNYHEKLDQREKERDAARKSWQAIGKIAELKEG |
| | | YLSAVIHQLTKLMVEYNAVVVMEDLNFGFKRGRFHVEKQVYQKFEHI |
| | | LIDKSNYLVFKDRGLNEPGGVLNGYQIAGQFESFQKLGKQSGMLFYVP |
| | | AGYTSKIDPKTGFVSMMNFKDLTNVHKKRDFFSKFDNIHYDEANGSFV |
| | | FTFDYKKFDGKAKEEMKLTKWSVYSRDKRIVYFAKTKSYEDVLPTEKL |
| | | QKIFESNGIDYKSGNNIQDSVMAIGADLKEGAKPSKEISDFWDGLLSNF |
| | | KLILQMRNSNARTGEDYIISPVMADDGTFFDSREEFKKGEDAKLPLDAD |
| | | ANGAYHIALKGLSLINKINLSKDEELKKFDMKISNADWFKFAQEKNYA |
| | | K |
| SEQ | Cas12 | MEEKKMSKIEKFIGKYKISKTLRFRAVPVGKTQDNIEKKGILEKDKKRS |
| ID | Variant | EDYEKVKAYLDSLHRDFIENTLKKVKLNELNEYACLFFSGTKDDGDKK |
| NO: | | KMEKLEEKMRKTISNEFCNDEMYKKIFSEKILSENNEEDVSDIVSSYKG |
| 42 | | FFTSLNGYVNNRKNLYVSDAKPTSIAYRCINENLPKFLRNVECYKKVV |
| | | QVIPKEQIEYMSNNLNLSPYRIEDCFNIDFFEFCLSQGGIDLYNTFIGGYS |
| | | KKDGTKVQGINEIVNLYNQKNKKDKEKYKLPQFTPLFKQILSDRDTKSF |
| | | SIEKLENIYEVVELVKKSYSDEMFDDIETVFSNLNYYDASGIYVKNGPAI |
| | | THISMNLTKDWATIRNNWNYEYDEKHSTKKNKNIEKYEDTRNTMYKK |
| | | IDSFTLEYISRLVGKDIDELVKYFENEVANFVMDIKKTYSKLTPLFDRCQ |
| | | KENFDISEDEVNDIKGYLDNVKLLESFMKSFTINGKENNIDYVFYGKFT |
| | | DDYDKLHEFDHIYNKVRNYITTSRKPYKLDKYKLYFDNPQLLGGWDIN |
| | | KEKDYRTVMLTKDGKYYFAIIDKGEHPFDNIPKDYFDNNGYYKKIIYR |
| | | QIPNAAKYLSSKQIVPQNPPEEVKRILDKKKADSKSLTEEEKNIFIDYIKS |
| | | DFLKNYKLLFDKNNNPYFNFAFRESSTYESLNEFFEDVERQAYSVRYEN |
| | | LPADYIDNLVNEGKIYLFEIYSKDFSEYSKGTNNLHTMYFKALFDNDNL |
| | | KNTVFKLSGNAELFIRPASIKKDELVIHPKNQLLQNKNPLNPKKQSIFDY |
| | | DLVKDKRFFENQYMLHISIEINKNERDAKKIKNINEMVRKELKDSDDNY |
| | | IIGIDRGERNLLYVCVINSAGKIVEQMSLNEIINEYNGIKHTVDYQGLLD |
| | | KCEKERNAQRQSWKSIENIKELKDGYISQVVHKLCQLVEKYDAIIAME |
| | | NLNGGFKRGRTKFEKQVYQKFENKLINKMEYMADKKRKTTENGGILR |
| | | GYQLTNGCINNSYQNGFIFYVPAWLTSKIDPTTGFVDLLKPKYTNVEEA |
| | | HLWINKFNSITYDKKLDMFAFNINYSQFPRADIDYRKIWTFYTNGYRIE |
| | | TFRNSEKNNEFDWKEVHLTSVIKKLLEEYQINYISGKNIIDDLIQIKDKPF |
| | | WNSFIKYIRLTLQMRNSITGRTDVDYIISPVINNEGTFYDSRKDLDEITLP |
| | | QDADANGAYNIARKALWIIEKLKESPDEELNKVKLAITQREWLEYAQI |
| | | NI |
| SEQ | Cas12 | MIIHNCYIGGSFMKKIDSFTNCYSLSKTLRFKLIPIGATQSNFDLNKMLD |
| ID | Variant | EDKKRAENYSKAKSIIDKYHRFFIDKVLSSVTENKAFDSFLEDVRAYAE |
| NO: | | LYYRSNKDDSDKASMKTLESKMRKFIALALQSDEGFKDLFGQNLIKKT |
| 43 | | LPEFLESDTDKEIIAEFDGFSTYFTGFFNNRKNMYSADDQPTAISYRCIN |
| | | DNLPKFLDNVRTFKNSDVASILNDNLKILNEDFDGIYGTSAEDVFNVDY |
| | | FPFVLSQKGIEAYNSILGGYTNSDGSKIKGLNEYINLYNQKNENIHRIPK |
| | | MKQLFKQILSERESVSFIPEKFDSDDDVLSSINDYYLERDGGKVLSIEKT |
| | | VEKIEKLFSAVTDYSTDGIFVKNAAELTAVCSGAFGYWGTVQNAWNN |
| | | EYDALNGYKETEKYIDKRKKAYKSIESFSLADIQKYADVSESSETNAEV |
| | | TEWLRNEIKEKCNLAVQGYESSKDLISKPYTESKKLFNNDNAVELIKNA |
| | | LDSVKELENVLRLLLGTGKEESKDENFYGEFLPCYERICEVDSLYDKVR |
| | | NYMTQKLYKTDKIKLNFQNPQFLGGWDRNKEADYSAVLLRRNSLYYI |
| | | AIMPSGYKRVFEKIPAPKADETVYEKVIYKLLPGPNKMLPKVFFSKKGI |
| | | ETFNPPKEILEKYELGTHKTGDGFNLDDCHALIDYFKSALDVHSDWSNF |
| | | GFRFSDTSTYKNIADFYNEVKNQGYKITFCDVPQSYINELVDEGKLYLF |
| | | QLYNKDFSEHSKGTPNLHTLYFKMLFDERNLENVVFKLNGEAEMFYRE |
| | | ASISKDDMIVHPKNQPIKNKNEQNSRKQSTFEYDIVKDRRYTVDQFML |
| | | HIPITLNFTANGGTNINNEVRKALKDCDKNYVIGIDRGERNLLYICVVDS |
| | | EGRIIEQYSLNEIINEYNGNTYSTDYHALLDKKEKERLESRKAWKTVENI |
| | | KELKEGYISQVVHKICELVEKYDAVIVMEDLNLGFKQGRSGKFEKSVY |
| | | QKFEKMLIDKLNYFADKKKSPEEIGSVLNAYQLTNAFESFEKMGKQNG |
| | | FIFYVPAYLTSKIDPTTGFADLLHPSSKQSKESMRDFVGRFDSITFNKTE |
| | | NYFEFELDYNKFPRCNTDYRKKWTVCTYGSRIKTFRNPEKNSEWDNKT |
| | | VELTPAFMALFEKYSIDVNGDIKAQIMSVDKKDFFVELIGLLRLTLQMR |
| | | NSETGKVDRDYLISPVKNSEGVFYNSDDYKGIENASLPKDADANGAYN |
| | | IARKGLWIIEQIKACENDAELNKIRLAISNAEWLEYAQKK |
| SEQ | Cas12 | MKEQFVNQYPISKTLRFSLIPIGKTEENFNKNLLLKEDEKKAEEYQKVK |
| ID | Variant | GYIDRYHKFFIETALCNINFEGFEEYSLLYYKCSKDDNDLKTMEDIEIKL |
| NO: | | RKQISKTMTSHKLYKDLFGENMIKTILPNFLDSDEEKNSLEMFRGFYTY |
| 44 | | FSGFNTNRKNMYTEEAKSTSIAYRCINDNLPKFLDNSKSFEKIKCALNK |
| | | EELKAKNEEFYEIFQIYATDIFNIDFFNFVLTQPGIDKYNGIIGGYTCSDG |
| | | TKVQGLNEIINLYNQQIAKDDKSKRLPLLKMLYKQILSDRETVSFIPEKF |
| | | SSDNEVLESINNYFSKNVSNAIKSLKELFQGFEAYNMNGIFISSGVAITDL |
| | | SNAVFGDWNAISTAWEKAYFETNPPKKNKSQEKYEEELKANYKKIKSF |
| | | SLDEIQRLGSIAKSPDSIGSVAEYYKITVTEKIDNITELYDGSKELLNCNY |
| | | SESYDKKLIKNDTVIEKVKTLLDAVKSLEKLIKPLVGTGKEDKDELFYG |
| | | TFLPLYTSLSAVDRLYDKVRNYATQKPYSKDKIKLNFNCSSFLSGWAT |
| | | DYSSNGGLIFEKDGLYYLGIVNKKFTTEEIDYLQQNADENPAQRIVYDF |
| | | QKPDNKNTPRLFIRSKGTNYSPSVKEYNLPVEEIVELYDKRYFTTEYRN |
| | | KNPELYKASLVKLIDYFKLGFTRHESYRHYDFKWKKSEEYNDISEFYK |
| | | DVEISCYSLKQEKINYNTLLNFVAENRIYLFQIYNKDFSKYSKGTPNLHT |
| | | RYFKALFDENNLSDVVFKLNGGSEMFFRKASIKDNEKVVHPANQPIDN |
| | | KNPDNSKKQSTFDYELIKDKRFTKHQFSIHIPITMNFKARGRDFINNDIR |
| | | KAIKSEYKPYVIGIDRGERNLIYISVINNNGEIVEQMSLNDIISDNGYKVD |
| | | YQRLLDRKEKERDNARKSWGTIENIKELKEGYISQVIHKICELVIKYDA |
| | | VIAMEDLNFGFKRGRFNVEKQVYQKFENMLISKLNYLCDKKSEANSEG |
| | | GLLKAYQLTNKFDGVNKGKQNGIIFYVPAWLTSKIDPVTGFVDLLHPK |
| | | YISVEETHSLFEKLDDIRYNFEKDMFEFDIDYSKLPKCNADFKQKWTVC |
| | | TNADRIMTFRNSEKNNEWDNKRILLSDEFKRLFEEFGIDYCHNLKNKIL |
| | | SISNKDFCYRFIKLFALTMQMRNSITGSTNPEDDYLISPVRDENGVFYDS |
| | | RNFIGSKAGLPIDADANGAYNIARKGLWAINAIKSTADDMLDKVDLSIS |
| | | NAKWLEYVQK |
| SEQ | Cas12 | MADLSQFTHKYQVPKTLRFELIPQGKTLENLSAYGMVADDKQRSENY |
| ID | Variant | KKLKPVIDRIYKYFIEESLKNTNLDWNPLYEAIREYRKEKTTATITNLKE |
| NO: | | QQDICRRAIASRFEGKVPDKGDKSVKDFNKKQSKLFKELFGKELFTDSV |
| 45 | | LEQLPGVSLSDEDKALLKSFDKFTTYFVGFYDNRKNVFSSDDISTGIPHR |
| | | LVQENFPKFIDNCDDYKRLVLVAPELKEKLEKAAEATKIFEDVSLDEIFS |
| | | IKFYNRLLQQNQIDQFNQLLGGIAGAPGTPKIQGLNETLNLSMQQDKTL |
| | | EQKLKSVPHRFSPLYKQILSDRSSLSFIPESFSCDAEVLLAVQEYLDNLK |
| | | TEHVIEDLKEVFNRLTTLDLKHIYVNSTKVTAFSQALFGDWNLCREQLR |
| | | VYKMSNGNEKITKKALGELESWLKNSDIAFTELQEALADEALPAKVNL |
| | | KVQEAISGLNEQMAKSLPKELKIPEEKEELKALLDAIQEVYHTLEWFIVS |
| | | DDVETDTDFYVPLKETLQIIQPIIPLYNKVRNFATQKPYSVEKFKLNFAN |
| | | PTLADGWDENKEQQNCAVLFQKGNNYYLGILNPKNKPDFDNVDTEKQ |
| | | GNCYQKMVYKQFPDFSKMMPKCTTQLKEVKQHFEGKDSDYILNNKNF |
| | | IKPLTITREVYDLNNVLYDGKKKFQIDYLRKTKDEDGYYTALHTWIDF |
| | | AKKFVASYKSTSIYDTSTILPPEKYEKLNEFYGALDNLFYQIKFENIPEEII |
| | | DTYVEDGKLFLFQIYNKDFAAGATGAPNLHTIYWKAVFDPENVKDVV |
| | | VKLNGQAELFYRPKSNMDVIRHKVGEKLVNRTLKDGSILTDELHKELY |
| | | LYANGSLKKGLSEDAKIILDKNLAVIYDVHHEIVKDRRFTTDKFFFHVP |
| | | LTLNYKCDKNPVKFNAEVQEYLKENPDTYVIGIDRGERNLIYAVVIDPK |
| | | GRIVEQKSFNVINGFDYHGKLDQREKERVKARQAWTAVGKIKELKQG |
| | | YLSLVVHEISKMMVRYQAVVVLENLNVGFKRVRSGIAEKAVYQQFEK |
| | | MLINKLNYLMFKDAGGTEPGSVLNAYQLTDRFESFAKMGLQTGFLFYI |
| | | PAAFTSKIDPATGFVDPFRWGAIKTLADKREFLSGFESLKFDSTTGNFIL |
| | | HFDVSKNKNFQKKLEGFVPDWDIIIEANKMKTGKGATYIAGKRIEFVRD |
| | | NNSQGHYEDYLPCNALAETLRQCDIPYEEGKDILPLILEKNDSKLLHSVF |
| | | KVVRLTLQMRNSNAETGEDYISSPVEDVSGSCFDSRMENEKLPKDADA |
| | | NGAYHIALKGMLALERLRKDEKMAISNNDWLNYIQEKRA |
| SEQ | Cas12 | MTNFDNFTKKYVNSKTIRLEAIPVGKTLKNIEKMGFIAADRQRDEDYQ |
| ID | Variant | KAKSVIDHIYKAFMDDCLKDLFLDWDPLYEAVVACWRERSPEGRQAL |
| NO: | | QIMQADYRKKIADRFRNHELYGSLFTKKIFDGSVAQRLPDLEQSAEEKS |
| 46 | | LLSNFNKFTSYFRDFFDKRKRLFSDDEKHSAIAYRLINENFLKFVANCEA |
| | | FRRMTERVPELREKLQNTGSLQVYNGLALDEVFSADFYNQLIVQKQID |
| | | LYNQLIGGIAGEPGTPNIQGLNATINLALQGDSSLHEKLAGIPHRFNPLY |
| | | KQILSDVSTLSFVPSAFQSDGEMLAAVRGFKVQLESGRVLQNVRRLFN |
| | | GLETEADLSRVYVNNSKLAAFSSMFFGRWNLCSDALFAWKKGKQKKI |
| | | TNKKLTEIKKWLKNSDIAIAEIQEAFGEDFPRGKINEKIQAQADALHSQL |
| | | ALPIPENLKALCAKDGLKSMLDTVLGLYRMLQWFIVGDDNEKDSDFYF |
| | | GLGKILGSLDPVLVLYNRVRNYITKKPYSLTKFRLNFDNSQLLNGWDE |
| | | NNLDTNCASIFIKDGKYYLGISNKNNRPQFDTVATSGKSGYQRMVYKQ |
| | | FANWGRDLPHSTTQMKKVKKHFSASDADYVLDGDKFIRPLIITKEIFDL |
| | | NNVKFNGKKKLQVDYLRNTGDREGYTHALHTWINFAKDFCACYKSTS |
| | | IYDISCLRPTDQYDNLMDFYADLGNLSHRIVWQTIPEEAIDNYVEQGQL |
| | | FLFQLYNKDFAPGADGKPNLHTLYWKAVFNPENLEDVVVKLNGKAEL |
| | | FYRPRSNMDVVRHKVGEKLVNRKLKNGLTLPSRLHEEIYRYVNGTLNK |
| | | DLSADARSVLPLAVVRDVQHEIIKDRRFTADKFFFHASLTFNFKSSDKP |
| | | VGFNEDVREYLREHPDTYVVGVDRGERNLIYIVVIDPQGNIVEQRSFNM |
| | | INGIDYWSLLDQKEKERVEAKQAWETVGKIKDLKCGYLSFLIHEITKIII |
| | | KYHAVVILENLSLGFKRVRTGIAEKAVYQQFERMLVTKLGYVVFKDRA |
| | | GKAPGGVLNAYQLTDNTRTAENTGIQNGFLFYVPAAFTSRVDPATGFF |
| | | DFYDWGKIKTATDKKNFIAGFNSVRYERSTGDFIVHVGAKNLAVRRVA |
| | | EDVRTEWDIVIEANVRKMGIDGNSYISGKRIRYRSGEQGHGQYENHLPC |
| | | QELIRALQQYGIQYETGKDILPAILQQDDAKLTDTVFDVFRLALQMRNT |
| | | SAETGEDYFNSVVRDRSGRCFDTRRAEAAMPKEADANDAYHIALKGLF |
| | | VLEKLRKGESIGIKNTEWLRYVQQRHS |
| SEQ | Cas12 | MENYGGFTGLYPLQKTLKFELRPQGRTMEHLVSSNFFEEDRDRAEKYK |
| ID | Variant | IVKKVIDNYHKDFINECLSKRSFDWTPLMKTSEKYYASKEKNGKKKQD |
| NO: | | LDQKIIPTIENLSEKDRKELELEQKRMRKEIVSVFKEDKRFKYLFSEKLFS |
| 47 | | ILLKDEDYSKEKLTEKEILALKSFNKFSGYFIGLHKNRANFYSEGDESTA |
| | | IAYRIVNENFPKFLSNLKKYREVCEKYPEIIQDAEQSLAGLNIKMDDIFP |
| | | MENFNKVMTQDGIDLYNLAIGGKAQALGEKQKGLNEFLNEVNQSYKK |
| | | GNDRIRMTPLFKQILSERTSYSYILDAFDDNSQLITSINGFFTEVEKDKEG |
| | | NTFDRAVGLIASYMKYDLSRVYIRKADLNKVSMEIFGSWERLGGLLRIF |
| | | KSELYGDVNAEKTSKKVDKWLNSGEFSLSDVINAIAGSKSAETFDEYIL |
| | | KMRVARGEIDNALEKIKCINGNFSEDENSKMIIKAILDSVQRLFHLFSSF |
| | | QVRADFSQDGDFYAEYNEIYEKLFAIVPLYNRVRNYLTKNNLSMKKIK |
| | | LNFKNPALANGWDLNKEYDNTAVIFLREGKYYLGIMNPSKKKNIKFEE |
| | | GSGTGPFYKKMAYKLLPDPNKMLPKVFFAKKNINYYNPSDEIVKGYKA |
| | | GKYKKGENFDIDFCHKLIDFFKESIQKNEDWRAFNYLFSATESYKDISDF |
| | | YSEVEDQGYRMYFLNVPVANIDEYVEKGDLFLFQIYNKDFASGAKGNK |
| | | DMHTIYWNAAFSDENLRNVVVKLNGEAELFYRDKSIIEPICHKKGEML |
| | | VNRTCFDKTPVPDKIHKELFDYHNGRAKTLSIEAKGYLDRVGVFQASY |
| | | EIIKDRRYSENKMYFHVPLKLNFKADGKKNLNKMVIEKFLSDKDVHIIG |
| | | IDRGERNLLYYSVIDRRGNIIDQDSLNIIDGFDYQKKLGQREIERREARQ |
| | | SWNSIGKIKDLKEGYLSKAVHKVSKMVLEYNAIVVLEDLNFGFKRGRF |
| | | KVEKQVYQKFEKMLIDKLNYLVFKEVLDSRDAGGVLNAYQLTTQLES |
| | | FNKLGKQSGILFYVPAAYTSKIDPTTGFVSLFNTSRIESDSEKKDFLSGFD |
| | | SIVYSAKDGGIFAFKFDYRNRNFQREKTDHKNIWTVYTNGDRIKYKGR |
| | | MKGYEITSPTKRIKDVLSSSGIRYDDGQELRDSIIQSGNKVLINEVYNSFI |
| | | DTLQMRNSDGEQDYIISPVKNRNGEFFRTDPDRRELPVDADANGAYHI |
| | | ALRGELLMQKIAEDFDPKSDKFTMPKMEHKDWFEFMQTRGD |
| SEQ | Cas12 | MLHAFTNQYQLSKTLRFGATLKEDEKKCKSHEELKGFVDISYENMKSS |
| ID | Variant | ATIAESLNENELVKKCERCYSEIVKFHNAWEKIYYRTDQIAVYKDFYRQ |
| NO: | | LSRKARFDAGKQNSQLITLASLCGMYQGAKLSRYITNYWKDNITRQKS |
| 48 | | FLKDFSQQLHQYTRALEKSDKAHTKPNLINFNKTFMVLANLVNEIVIPL |
| | | SNGAISFPNISKLEDGEESHLIEFALNDYSQLSELIGELKDAIATNGGYTP |
| | | FAKVTLNHYTAEQKPHVFKNDIDAKIRELKLIGLVETLKGKSSEQIEEYF |
| | | SNLDKFSTYNDRNQSVIVRTQCFKYKPIPFLVKHQLAKYISEPNGWDED |
| | | AVAKVLDAVGAIRSPAHDYANNQEGFDLNHYPIKVAFDYAWEQLANS |
| | | LYTTVTFPQEMCEKYLNSIYGCEVSKEPVFKFYADLLYIRKNLAVLEHK |
| | | NNLPSNQEEFICKINNTFENIVLPYKISQFETYKKDILAWINDGHDHKKY |
| | | TDAKQQLGFIRGGLKGRIKAEEVSQKDKYGKIKSYYENPYTKLTNEFK |
| | | QISSTYGKTFAELRDKFKEKNEITKITHFGIIIEDKNRDRYLLASELKHEQ |
| | | INHVSTILNKLDKSSEFITYQVKSLTSKTLIKLIKNHTTKKGAISPYADFH |
| | | TSKTGFNKNEIEKNWDNYKREQVLVEYVKDCLTDSTMAKNQNWAEF |
| | | GWNFEKCNSYEDIEHEIDQKSYLLQSDTISKQSIASLVEGGCLLLPIINQD |
| | | ITSKERKDKNQFSKDWNHIFEGSKEFRLHPEFAVSYRTPIEGYPVQKRY |
| | | GRLQFVCAFNAHIVPQNGEFINLKKQIENFNDEDVQKRNVTEFNKKVN |
| | | HALSDKEYVVIGIDRGLKQLATLCVLDKRGKILGDFEIYKKEFVRAEKR |
| | | SESHWEHTQAETRHILDLSNLRVETTIEGKKVLVDQSLTLVKKNRDTPD |
| | | EEATEENKQKIKLKQLSYIRKLQHKMQTNEQDVLDLINNEPSDEEFKKR |
| | | IEGLISSFGEGQKYADLPINTMREMISDLQGVIARGNNQTEKNKIIELDA |
| | | ADNLKQGIVANMIGIVNYIFAKYSYKAYISLEDLSRAYGGAKSGYDGR |
| | | YLPSTSQDEDVDFKEQQNQMLAGLGTYQFFEMQLLKKLQKIQSDNTVL |
| | | RFVPAFRSADNYRNILRLEETKYKSKPFGVVHFIDPKFTSKKCPVCSKTN |
| | | VYRDKDDILVCKECGFRSDSQLKERENNIHYIHNGDDNGAYHIALKSV |
| | | ENLIQMK |
| SEQ | Cas12 | MKNGINLFKTKTTKTKGVDMEKYQITKTIRFKLLPDNAHEIVEKVKSLK |
| ID | Variant | TSNVDELMDEVKNVHLKGLELLFALKKYFYFDGNQCKSFKSTLEIKAR |
| NO: | | WLRLYTPDQYYLKKSSKNSYQLKSLSYFKDVFNDWLFNWEESVSELAI |
| 49 | | IYEKYKICQHQRDSRADIALLIKKLSMKEYFPFISDLIDCVNDKNSNKTF |
| | | LMKLSEELSVLLEKCNSRALPYQSNGIVVGKASLNYYTVSKSEKMLQN |
| | | EYEDVCQSLDKNYDITEMKVILYKEKLDNLNFKDVTIANAYNLLKENK |
| | | ALQKRLFSEYVSQGKVLSLIKTELPLFSNINDNDFEKYKEWSNEIKKLA |
| | | DKKNTFCKKTQQDKIKDIQNKISELKKKRGALFQYKFTSFQKHCDNYK |
| | | KVAVQYGKLKARKKAIEKDEIEANLLRYWSVILEQEDKHSLVLIPKNN |
| | | AKDAKQYIETINTKGGKYIIHHLDSLTLRALNKLCFNAVDIEKGQMVRE |
| | | NTFYQGIKEEFERNKINCDNQGVLKIQGLYSFKTEGGQINEKEAVEFFK |
| | | EVLKSNYAREVLNLPYDLESNIFQKEYTNLDQFRQDLEKCCYALHSKIG |
| | | KDDLDEFTRRFEAQVFDITSIDLKSKKEKTKTTGEMKKHTQLWLEFWK |
| | | GAIEQNFATRVNPELSIFWRAPKSSREKKYGKGSDLYDPNKNNRYLYE |
| | | QYTLALTITENAGSHFKDIAFKDTSKIKEAIKEFNMSLSQSKYCFGIDRG |
| | | NAELVSLCLIKNEKDFPFEKFPVYRLRDLTYQGDFKDKHDQMRYGVAI |
| | | KNISYFIDQEDLFEKNNLSAIDMTTAKLIKNKIVLNGDVLTYLKLKEETA |
| | | KHKLTQFFQGSSINKNSRVYFDEDENVFKITTNRNHNPEEIIYFYRGEYG |
| | | AIKNKNDLEDILNEYLCKMETGESEIVLLNRVNHLRDAISANIVGILSYLI |
| | | DLFPETIVALENLAKGTIDRHVSQSYENITRRFEWALYRKLLNKQLAPP |
| | | ELKENILLREGDDKIDQFGIIHFVEEKNTSKDCPNCRKTTQQTNDNKFKE |
| | | KKFVCKSCGFDTSKDRKGMDSLNSPDTVAAYNVARKKFES |
| SEQ | Cas12 | MAKETKEFKTFDDFTNLYEVQKTLRFELEAVPETEIVLENRGIWYKRD |
| ID | Variant | KKRADEKPIVKFYMDILHREFTDEALEKIKESGVLNLSGYFKLFEELRRL |
| NO: | | QNHGANTKEEKKLKLEEIRAKKREISNELSQIRRVFSVRGFDVVDSDWK |
| 50 | | KKYTIEGKKIKNDKSKTYLILSENILNFLENRFTSKEVERLRSIDKKHVE |
| | | DYGNVVNSGGENIFATFKGFFGYFDSLIKNRENFYETDGKAGRVATRS |
| | | VDENLNFFAENLHIFSTDLPKALKDDLSDTQKAIFERSYYKNCLLQKDI |
| | | KSYNLIIGDINKEINKHRQQRDTKIKFLNTLFKQILSIEEKEQYKHIEINND |
| | | EDLIRAIRDFISLNESKISEGTKIFNQFIQRCLQKEDLGQIYLPKDSVNTIA |
| | | HRIFKPWDEIMALFDRKYFVSLEEIKDLTESSVWKERVLEESKTKSLIFK |
| | | DTHIHTIISGQEIFSNFILILEKEYKNQFSGFISETRRGKAAFVGYDESLKN |
| | | LRATIKWFEGKNLKLSETEKVEWIKAIKDYADAALRIFQMTKYLWLPV |
| | | VGDEEDKDYLRIKAEIDQLTKDNDFYNKINAFIDGYKPEPFIYRSSFQEY |
| | | LTRRPFSTDKFKINFENSRLLDGWDKDMIDDRMGILLQRDGDYFLGILN |
| | | KEDRHCLDNLVDVKSEDKNSYALMQFKQLTGLYRQLPRMAFPKKKQP |
| | | VLEANAEIKKIKEDFDFLQKQKKEREVNVNVVFDNKKLNLLINHYAEF |
| | | LKENYKDEKCYDFSLLNKEKVYESLSDFYADVDKITYSLSFIQVSIDQLI |
| | | KTGKILLFRLKNKDLLKGSLGQNKNLHTYYFHALFERENLSQGRIRLGA |
| | | QAEIFFRPASIEKEKDKNRSNALKKSPKTRYVKEILKNKRYSEDKVFLH |
| | | LPIQLNADAYDLPSINQNVFEFIKNRQEKVKIIGIDRGEKNLAYYSVISQN |
| | | SNGKIKIEEPPRDLNLGYLEPLDELENKRQDERKAWQSISEIKSKRDGYI |
| | | SYAVSKIVELMLKYQAIIVLEDLSGKFKRSRMKFEKAPYQQLELALIKK |
| | | LNYLVKKNSKSGKPGHYLSAYQLTEPVGSYKEMGKQTGIIFYTQAGYT |
| | | SRTCPTCGWRKRVQGLYYKDRTSAQRRFDPKTGVKIFYDSVNDRFVFQ |
| | | YHPVYEQKELKEWDKEIYSDVTRIRWNNEEKKNNEYRKGDITLKIKRL |
| | | FRDRGIDLSRNINEQLVNVGDASFWEELINLLRLITEIRNIDNENNRDFIE |
| | | CPHCHFQSENGFHGVAWNGDANGAYNIARKGLLITKAVCDPEKNVGD |
| | | ITWSDLKVDMKDWDAATDEWAKKNPEK |
| SEQ | Cas12 | MENEKIFSDLTNRYQVVKTLPFELKPVPRTRVLLGLDNPNKGEIFSKDR |
| ID | Variant | ERAENFTIIKKYIDRLHSLFINESLKKADIDFSNFYKQYGKNINTKNNKNI |
| NO: | | DDDNDINDDEKEDSENDNLKKYRQEIANLFNKSKYKSWVNVGKDGDK |
| 51 | | ISGMLFEKGLIDLLRTHFSDNLNEDIEIPELFSNKKIKDTRKLKEIINSFGK |
| | | DGKDGQNFTTYFSVSFHNNRKNYYKSDGKMGRVSTRIVDENLERFCK |
| | | NIYLYKELIGKNEIKEIFSGNWDIYLQKKPNFSNDKTYKKLDEFKNDKY |
| | | DWEMIFRDVNSYNKYFLQSDIEFYNYIRGKLNQDINEYNGKKRDSKEKI |
| | | NSQFENLRNQVHGEKKNYDDDFEIDEDNIIQFINEIFVRHNQNKMRFSE |
| | | KLFSDFIDLLMVDNGDKLDKVYFSQKAVENAIARYYFVEETTNEGREP |
| | | LLISLLLQNAGKDRKKLSNKPIKLGDIKFVLDQANNKPAEDIFKNRYVL |
| | | SESNNDGIINANDKNHWANLLRLIKKDFYFHKDNLIKSQDKLALETKY |
| | | NKGSDEGERQIETIKNFAESAKAILRMTKYFDLRKNGVIQNVIGGKDPIH |
| | | EEVDKYFDGDVLSGEESCRISKYYDALRNFITKKAWSADKIILNFDCSEF |
| | | LGGWDRSQEQKKRGIILRHRDGDEERYYLAVLGKNGKQYFENRTLFK |
| | | GCESSDWQKIEYNVIQKPHMSLPKNLITPFFKKDKITNERFIDRSKKGAK |
| | | ALIEIDINPSDEFLNNYNLGKHTKENLDKSFLCDYFKYLMDAIAKYYKG |
| | | EFNFNFPDVSNFDNTQPFYSFIEKNAYSIKYFGISSKEIEKLIADCYYKED |
| | | VYLFQIYCKDFEIDPKIGKAKYGNEFRTKAEIRKSKGEEAGNENLNTKY |
| | | FKLLFDEKNLKNQNGIVYKLNGGAKMFYRPSSIKKDEKIDGKWRYKED |
| | | KYSLNITITCNFSSKKDDLSIDKDINKKIAEVNANSDFRIISIDRGEKNLA |
| | | YCCVMDENANILDIKSLNRITRYDKNGKAIKEKNMFHEVKDGKLCYGE |
| | | PVYDFYKDYQNLLDEREIKRLVNRRSWNVIEDIKNLKKGYVALLINYIC |
| | | KAVVIAINEGKYPIIVLESLDKGMLHNRVKIEKQIYRGVEEGLVRKLNY |
| | | FVDKKTDNVLNAWQLLAKFETVGSSLDRKKQLGIIFYVDPGYTSITCPC |
| | | CGFRQRKYIKAERAEENFKEIKIKFDGKRYSFAYDYRCIDDNGKEKSKE |
| | | DIIYSNVKRLLRSGRNGRAVQIEDVTDELTNLFKKHNINIEQDINEQLAG |
| | | KDNKFWKQLLWWFNAIEQIRNTQSLRRKFNTEENKLEILENNDCDFILC |
| | | PHCYFDSNKDKFQNKIWNGDANGAFNIGRKGIIDIFEIKKHQRMLSDFM |
| | | EQWGIDKLPKANGGNQAVIEIVKNDKKYNLCILNNKKIPYYCLRIGKEK |
| | | IDSIADDRKCNQLPDLMVNWKKWDMWLDKWGK |
| SEQ | Cas12 | MPEVKNVFQDFTNLYELSKTLRFELKPVPETEKILELNAAKTKKFPKDL |
| ID | Variant | YRAENFEIIKKYTDELHRTYIRETLNNVNIDYLKFLEIFRINGKKKNEMT |
| NO: | | DENEESDENNEKDDIQKIKKELRSKIGNLFNKWNNDKDNKFKDWVKID |
| 52 | | VGKKEKEVSGDLFGKELITILKNYFKNKLDSKVNVPMLFFNEQEIKNGE |
| | | AKKQRKLEAVFENFDKFTTYFTDSFYNNRKNYYKTEGRVGQVATRIID |
| | | ENLPRFCSNLIAFNEVVSLYSTLLNNFDLGWKEYLNEKKINQTWVEKFE |
| | | LSNYDWKALFNDVNYYNQCLLQEGIDKYNYIIKKLNKDINEYTQNKYK |
| | | SVEKGNNNNPDINFFQKLHKQIHGERDFKLIEIDIDENNIFTKILPEFILHS |
| | | DMKLMTKIDEEVGVEEIVGAERIIKIFIKQELKDLEKIYLSRRAIETISAK |
| | | WFHSWETLKDLILGYLNKDLLESKKRKKVPDFVDFNIIKIVLENNKDDY |
| | | KDLFKRKYFEADKNEFVDWIDSSGGTKKLEFGGENWINFLNVFEYEFG |
| | | TLLTEYKKNKNALLYLIDKKIDYDKNNEVGQTAAIKNFADSALGIFRM |
| | | VSYFALRKKGVMVEPKNGKDEIFYAFVDRYLDGDDNDREEQNKIVQY |
| | | YNTLRNFVTQKAWSIDKVRLCFDCGEFLKGWDKDKIHERLGIILRNNN |
| | | KFYLGILNKNHKQIFIKIKSHDNNNFYYVIYDYKQLNNVYRQIPRLAFPS |
| | | RSVKKGDAYMLRAIQERKKKFFLEDEEFIELQEIKNEYDKIGNDLSKEK |
| | | LTKLIEYYKKVVISNYSSLYNVSNLNNKKFNSINEFNQYVENLMYSLIPT |
| | | RISPDFIKEKISKGELYLFQIYNKDFELDESIGKEKFGEDFAPVIMDGKNN |
| | | LHTEYFKLLFNDSNLKNPNGVVFKLSGGAKMFYRPATENLPIKKDRDG |
| | | NIIKNKKGENVIVGQRYKEDKYFLHLPIILNFVNKGKNYSINDMVNKAI |
| | | TNASDDQDKFRIIGLDRGEKHLVYYSVINERQEIIEIGSLNNISRKDNKGE |
| | | IIEEKNWYHDKFGNIEKEPTKEYHKDYHNLLDQREIERLKSRQSWEKIE |
| | | NIKELKEGYISAVINKICNLVIKAIKENKIPIVALENLNSGMKRGRIKIDK |
| | | QIYQKLELKLAKKLNFLVDKKEKNYLSAWQFTPKIETFSGDIEKKNQV |
| | | GIIFYVDPAFTSATCPNCGFRKRIKMDPQNAKKKIKDMEITYENGIYKFD |
| | | YPIENGENDVVYSDVERLKWDNEKKKVIKTKNVSDDFGKLFEDIKDKN |
| | | NLKKELLSIGEENKEFWKEFSRCFNLLLRIRNSKLIKRKLNDDTGKVEII |
| | | ADDDLADRDRDFIYCPQCHFHSEGGDVFGEFVKKKYLGKDNFEFNGD |
| | | ANGAYNIARKTIIAVNKIKDYQLGLNHFIEKYRISELPNNGKDKKNIFYN |
| | | NNSYILSFFEVQDEKFRKVKVYGLKKDGDRQIIQKKEMWYRRYPDIFV |
| | | NNKEWDKFVQNKS |
| SEQ | Cas12 | MLFFMSTDITNKPREKGVFDNFTNLYEFSKTLTFGLIPLKWDDNKKMIV |
| ID | Variant | EDEDFSVLRKYGVIEEDKRIAESIKIAKFYLNILHRELIGKVLGSLKFEKK |
| NO: | | NLENYDRLLGEIEKNNKNENISEDKKKEIRKNFKKELSIAQDILLKKVGE |
| 53 | | VFESNGSGILSSKNCLDELTKRFTRQEVDKLRRENKDIGVEYPDVAYRE |
| | | KDGKEETKSFFAMDVGYLDDFHKNRKQLYSVKGKKNSLGRRILDNFEI |
| | | FCKNKKLYEKYKNLDIDFSEIERNFNLTLEKVFDFDNYNERLTQEGLDE |
| | | YAKILGGESNKQERTANIHGLNQIINLYIQKKQSEQKAEQKETGKKKIK |
| | | FNKKDYPTFTCLQKQILSQVFRKEIIIESDRDLIRELKFFVEESKEKVDKA |
| | | RGIIEFLLNHEENDIDLAMVYLPKSKINSFVYKVFKEPQDFLSVFQDGAS |
| | | NLDFVSFDKIKTHLENNKLTYKIFFKTLIKENHDFESFLILLQQEIDLLID |
| | | GGETVTLGGKKESITSLDEKKNRLKEKLGWFEGKVRENEKMKDEEEGE |
| | | FCSTVLAYSQAVLNITKRAEIFWLNEKQDAKVGEDNKDMIFYKKFDEF |
| | | ADDGFAPFFYFDKFGNYLKRRSRNTTKEIKLHFGNDDLLEGWDMNKEP |
| | | EYWSFILRDRNQYYLGIGKKDGEIFHKKLGNSVEAVKEAYELENEADF |
| | | YEKIDYKQLNIDRFEGIAFPKKTKTEEAFRQVCKKRADEFLGGDTYEFK |
| | | ILLAIKKEYDDFKARRQKEKDWDSKFSKEKMSKLIEYYITCLGKRDDW |
| | | KRFNLNFRQPKEYEDRSDFVRHIQRQAYWIDPRKVSKDYVDKKVAEGE |
| | | MFLFKVHNKDFYDFERKSEDKKNHTANLFTQYLLELFSCENIKNIKSKD |
| | | LIESIFELDGKAEIRFRPKTDDVKLKIYQKKGKDVTYADKRDGNKEKEV |
| | | IQHRRFAKDALTLHLKIRLNFGKHVNLFDFNKLVNTELFAKVPVKILGM |
| | | DRGENNLIYYCFLDEHGEIENGKCGSLNRVGEQIITLEDDKKVKEPVDY |
| | | FQLLVDREGQRDWEQKNWQKMTRIKDLKKAYLGNVVSWISKEMLSGI |
| | | KEGVVTIGVLEDLNSNFKRTRFFRERQVYQGFEKALVNKLGYLVDKKY |
| | | DNYRNVYQFAPIVDSVEEMEKNKQIGTLVYVPASYTSKICPHPKCGWR |
| | | ERLYMKNSASKEKIVGLLKSDGIKISYDQKNDRFYFEYQWEQEHKSDG |
| | | KKKKYSGVDKVFSNVSRMRWDVEQKKSIDFVDGTDGSITNKLKSLLK |
| | | GKGIELDNINQQIVNQQKELGVEFFQSIIFYFNLIMQIRNYDKEKSGSEA |
| | | DYIQCPSCLFDSRKPEMNGKLSAITNGDANGAYNIARKGFMQLCRIREN |
| | | PQEPMKLITNREWDEAVREWDIYSAAQKIPVLSEEN |
| SEQ | Cas12 | MTIKKHKPFTNFECLTPVQKTLRFRLIPVGRTTEFVKCRNIIEADRKRSE |
| ID | Variant | MYPLLKELADRFYREFMTDQLSNLLFDWSPLVEALLLARNNTDPRENQ |
| NO: | | RIASLVRDEQKKYRTLLLKRLSGQVDRNGTPLPKNTASVNKKYYDDLF |
| 54 | | KARFVTETLPAYLEHLKNKPDGRISDELFDAYKDALDSYQKFTSRLTNF |
| | | WQARKNIFTDEDIATGFAYRIVHEIVPDYLFNRRVYEQHKLDFPEPLDL |
| | | LETELKKKNLIANDESLDALFTIPAINRLLTQKGVDLHNAVIGGFFTDDH |
| | | TKVQGFNELANLKNQTLKNVSDNSEIKPVGKMTRLKKHILSISESTSFLF |
| | | EQIESDDDLLARIIEFNNTLSEPDIDGLSIADINDQLYNIMTGVDPSTILVH |
| | | ARNLNKLSHEASLSWNRLRDGLYQMATESPYREDERFKRYIDASEEER |
| | | DLSKLKNDIYFSLQELQFALDQSIDLEEEATPTEDIFLPFEFPGMDLKSEL |
| | | TVLFRSIEQLISSETKLIGNPDAIATIKKYLDAIMARYSIWNLLSCEAVEL |
| | | QDDLFYPEYDRVMGSLSNIILLYNLARNYLSRKPSSKEKFRLNFDKPTL |
| | | ADGWSESKVPDNFSVLLRKDDLFYLGILKDRKAYRVLSYENCDETAKN |
| | | IKGYYERMIYHFSPDAYRMIPKCSTARKDVKKHFGEQGETTGYTLYPG |
| | | ASNFVKPFTIPYEIYRLQTELVNDKKRYQADYLKQTEDEEGYRQAVTA |
| | | WIDFCKSYLESYEGTSTFDYSHLLKSEDYEDVNQFYADVDRASYSIYFE |
| | | KVSVDLIHTMVDRGDLYLFQLYNKDFSPHSTGKPNLHTMYWRALFSN |
| | | DNLQNNTIKLNGQAELFYRPKQVEQPTVHLQGSYLLNRFDKHGDVIPA |
| | | GLYCEIYNHINERHPEGYTLSEEATQGLLDGRFVYREAPFELVKDKRYT |
| | | EDQLFLHVPLEFNWTASANVPFENLANEYIKKDSDLHIIGIDRGERNLLY |
| | | YSVINLQGDIVKQGSLNTLIQQTTLKGETVERQIPYQSMLKQREDERAE |
| | | ARQNWQSIDRIKDLKEGYLSHVIYKLSRLIIKYHAIVVMENLNVGFKRG |
| | | RFKVERQVYQKFEVALINKLNALSFKEYEPNELGGVMRPWQLARRVV |
| | | SPEDTRSQNGIVFYVPASYTSIVDPVTGFANLFYLNRIRNKDLNSFYGHF |
| | | QEIRYDHEFDRFIFRFNYADFGVFCRIKNVPSRTWNLVSGERKAFNPKR |
| | | RMIEKRDTTDEIKKALEAHGIAYQNEQNLLPLLLENENLLARIHRSFRLV |
| | | LQLRNSDSDRDDIVSPALDKENNTFDSGQQPYESSLPINADANGAYNIA |
| | | RKGLLLVDKVKNDKRAVLSNREWFEYLMAEE |
| SEQ | Cas12 | MENKDYSLSRFTKQYQNSKTVRFALTPIGRTEEYIIQNQYIEAARRKNQ |
| ID | Variant | AYKIVKPIIDEKFRSMIDDVLTHCEKQDWVTLDKLILQYQNNKCRENM |
| NO: | | DALAEQQEEIRKNISEEFTKSDEYKNFFGKEDSKKLFKIFLPEYLNQINA |
| 55 | | SESDKEAVNEFQKFKTYFSNFLIVRADIFKADNKHNTIPYRIVNENFMIF |
| | | AGNKRTFSNIIRLIPNALEEIAKDGMKKEEWSFYNIQNVDSWFEPDSFQ |
| | | MCMSQKGIQKYNFIIGLVNSYINLYTQQNPQATEVKRSRLKLRMLHKQI |
| | | LSDRVNPSWLPEQFKEGEEGEKQIYEAILALENDLIKNCFDKKYDLWIQ |
| | | SIDIQNPRIYIAASEMARVSSALHMGWNGLNDVRKTILLKSDKKQAKVE |
| | | KILKQDVSLKDLSDTLNRYADIYKEEQIPSLYQYIEYGSELLQDCAITRK |
| | | EYHDLLNGNSNTLSLNQNEKLIEGLKAYLDSYQAIVHFLNVFIVGDELD |
| | | KDTDFYAELDGLVESLSEIVPLYNKVRNYITRKVYSLDKMRIMFERSDF |
| | | LGGWGQSFDTKEALLFQKDNLYYIGIIEKKYTNMDVEYLHEGIKEGNR |
| | | AIRFIYNFQKADNKNIPRTFIRSKGTNYAPAVRKYNLPIESIIDIYDVGKF |
| | | KTNYKKINEKEYYESLEKLIDYFKDGILKNENYKKFHFNWKPSNEYENI |
| | | NEFYNDTNNACFLLEKEEINYDHLKEQANQGKIYLFQISSKDFNEGSKG |
| | | TPNLQTMYWRELFSNQNCKDGVIKLCGGASIYMRDASIKQPVVHRKN |
| | | AWLINKWYKVNGQNVVIPDNTYVKFTKIAQERMNEDELTPQERQLWN |
| | | SGLIQKKKATHDIMKDRRFTKKQYMLHAPLTINYKQQDSPRYFNEKVR |
| | | SFLKDNPDINIIGIDRGEKNLIYITIIDQKGNILKGMQKSFNQIEEKGKEGR |
| | | TIDYYSKLESVEARHDAARKNWKQIGTIRELKEGYLSQVVHEITQLMIQ |
| | | YNAVIVMENLNMGFKKGRMKVEKSVYQKFEKMLIDKMNYLAFKRDM |
| | | QGNAIDPYEVGGVMNGYQLTDRFTSFADMGSQNGFIFYVPAAYTSVID |
| | | PVTGFVNVFQKTEFKTNDFLHRFDSISWNDKEQSFVFTFDYQNFKCNGT |
| | | CYQNKWSLYADVDRIETIIKNNQVDRIEPCNPNQKLIDFFDKKGIIYRDG |
| | | HNIVDDLEKYDSKTISEIIHNFKLILQLRNSMRNPDTGEIIDYIASPVMHN |
| | | EERFDSRKRNPELPQDADANGAYHIALKGLMFLQKINEYADSDGNMD |
| | | NRKLKITNEEWFKYMQTRKEHTYF |
| SEQ | Cas12 | MSNKTSSITTTNKLSYTGFHNNGKQSKTLMFELKPIGRTTEHLDRKGYL |
| ID | Variant | ADDIDRAESYKTFKEIADNFHKNLIEESLATFTFSDTLKDYFDLWLSPVR |
| NO: | | TNEDTPKLRKMEAKLRKELSSALKQHPSFAATSSGKRLIDEALYPNASD |
| 56 | | KERQCLDRFKGRSSYLDSYTEVRSFIYTDLCKHNTIAYRVVNENLKIYL |
| | | ENILAYEKLMQTAVNGKLETVKEMFHDLYPTFSMDISIFFTSYGFDYCL |
| | | SQNAITRYNILLGGWSDDNGIHHKGLNNYINEYNQTVPRNKRLPKLNK |
| | | LQKMILSEENSMSFIIDKFENDVDLANAIRYWLKNCQFDALNLLIWTLD |
| | | VHYNLDEIHFKNDNQGKNISDLSQALFKNHHVIRDAWDYDYDIVNAK |
| | | AKSRQKPERYAEKRDKAFKKINSFSLSYLANILSQYDNQYANFVAQFK |
| | | TRISVHIQNVQQMIADKTLDMRLDPLMLLKSISSDTKLVEDIKRVLDSL |
| | | KDMQRMLTPLLGEGTEPNRDAMFYSDFEPLMNYVDTLTPLYNKVRNY |
| | | ITKKPYSTKKTSLYFGASNFGSGFDVTKLPVSHTIIMRDKGCYYLAVIDN |
| | | NKLIDKLYDHNDNDGYEYMVYKQIPSPIKYFSLKNILPQDPPDDIRQLLE |
| | | DRKNGAKWSHDDETRFIDYIVNEFLPTYPPIHDKNGNPYFSWKFKNPDE |
| | | YESLNEFFDDVSKQAYQTSFRFVSRDFVDDAVENGDIFFFQIYNQDFSP |
| | | ASHGKPSPHTLWFRALFSDVNLETKDIRLKGNATAYFRPASIFYTDEKW |
| | | RKGHHYEQLKNKFKFPIIKDKRYALDKFFFHITLEINCNATVEKYFNNR |
| | | VNEEIRKADRYNILAINRGERNLLYAVVMDQDGTILEQKSFNIIKSELPN |
| | | KTVKETDYWKKLHAREKERDTARKSWKSIECIKDLKKGYLSYVVKTIT |
| | | DMMFEYNAVLVMENLDIEMKRSRQKIEKNVYAQFQNAIIQKLSMYVN |
| | | KDIDLHIARTAPGGTLNPYQLTYIPASRTKTPKQNGFVFFLNPWNITEID |
| | | PTTGFVDLFQTCFRTKNEYKDFFAKFKDIRYNEAQGWFEFDTDYTYFR |
| | | DKEKAGKRTRWNICSYGTRLRRFRNPDKNYAEDAMTVYPTQMLKDLF |
| | | DEYNIPYAPASAKSTSISIKDDIIQIDKLDFYKKLLYILKLIVQLRNTSPSS |
| | | TEQEDDYIISPVINEDTNWFYDSRDYNEESLLPCNTDANGAYSLALKCN |
| | | MVIDRIKNTIPGEPVDMYISNADWLDARQ |
| SEQ | Cas12 | MNSKTSIFDFSNIFGRDITLRFKLTPVTINSKGEVKDANGADPYRPYLSA |
| ID | Variant | DEELQEQYELLKTAIDAYHQMYIDKKLKHILCLPLTEKGKDGVEHDTA |
| NO: | | KSKFVKSCLAYIKDYGEKDKKRQTADLRTFISRVFADDNISSLPPYKVK |
| 57 | | SDFITKTLRQWLEQPDTKVEKKEAILDLIEKNGSKLYANCQGLLEARQR |
| | | LYEKDGKSTSVPYRCIDRNLPRFSKDYHLFEKILGDCSDVFDFEQLDKD |
| | | FSEELKGIARLSGIRVESVREVFQPLLYLAYLNQEGIQYLNTIIGTKKEKG |
| | | TSALGLNEYINQYNQKQGIKKKKDGIPMLNKLNNQILFGDEVFIETLAE |
| | | HKEAIPVIKKVVSSLGKLGAFDGECHENKLYQFLLSLSSYAGNIYVNTK |
| | | VVAQISSSLWGDYSILYDAVKHDKNGRLIQKSVTLGELNEKIERLKLED |
| | | NRDAFEYFRRSQVKDVVHGSSNVGVFEQLKNCYNDFVEKKILKCSFFS |
| | | EDQVLVIQRLFDSILSLQRIFKVFCPSLYEVDSDGLFVAKFSDYWNVLR |
| | | GFDKDYDLLRNLFKRKPYSTDKIRVHFGLSNLMDGFVDSWTDKKDKG |
| | | TQYNGYILRQAHSFVDENTSKELQEFQRYNYYLVISGNVRLFREKGNA |
| | | LVCEKKKEKLVASDEFSGFERFDYYQSSINNFNREFKRLTGRDRKSFTD |
| | | EILQNEGKKELKSTYIENLIKVAKSMKRLTALQNLVSDEKVRKYSENLD |
| | | YETLSAEIGQILATGRERKYVPVSTNEMKNLLKSSKNNKGEEVRTFMFR |
| | | ISNKDLSYAETMQKGERKSHGAENMHTMYFRALLDTLQNTFDIGTGTV |
| | | YFRKASDKRKMKYDEKNPTHRKGDELAFKNPYNKGKKKSVFGYDLIK |
| | | DRRYTKDSYLFHLSITQNYQKKGNAEDLNAMVRDYIRTQEDLRVIGID |
| | | RGERNLLYATMIDGEGHILAQKSFNVIGYQGTTASGESFQVETDYHQLL |
| | | NEKAEKMRSLQREWKEMDKIQDMKDGYLSVVVHELAKMVVENNAII |
| | | VMEDLNMGFMESRQSQLANVYQKFEEKLRNKLQFYVDKRKRNDEPSG |
| | | LYHALQLAGTETKDNQNGFIFYIPAWNTSKIDSVTGFVNLFNLKYTNIK |
| | | DAKAFFSTFEKIEKNVETGHYDFTFSYSSMARKKMAKRMDGTRDSWTI |
| | | STHGSRIVREQKGNYWEYREIESLTSEFDALFEKYSIDTRCRLKEAIDKC |
| | | GEAEFFKELIRLMKWTLQLRNYDDRGNDYIVSPVCYRGNEYYCSLDYD |
| | | NEEGMCISKIPCQMPKDADANGAFNIARKGLMLCERLKKGEKIGVIKG |
| | | TEWLQYVQNMSERYVGMV |
| SEQ | Cas12 | MINTMEQPKKSIWDEFTNLYSLQKTLRFELKPQGKTKELVRTLFINPEE |
| ID | Variant | HHHKLISDDLELSKNYKKVKKLIDCMHRNIINNVLSKHQFTGEELKKLD |
| NO: | | KNSNAEDNDTETDNADKKDPFAKIRERLTKALNEESKIMFDNKLLNPK |
| 58 | | KGKNKGECELKKWMDKAEDKYFELGNNEKIDKEAVKADMERLEGFF |
| | | TYFGGFNKNRENVYSSKKIATAIPFRIIHDNFPIFKKNIENYKKITEKHPE |
| | | LAKLLNEKGANEIFQLEHFNKCLTQDGIDVYNNEKLGIIAKEQGKEQDK |
| | | GINQLINEYAQKKNKEIKENAKGGEKPKKIKIAVFDKLKKQILSISKTKS |
| | | FQFEVFEDTSDIINGINKRYTFLTEAKEGMSIVDEIKKIIGSVGDEKYSLD |
| | | EIYLKEKFISTLSKKLFNYSRYIEVALEKWYDDRYDDKINKSGTDKRKFI |
| | | SAKQFSITSIQDAINYYLEKYEKDEELSKKYTGKNIIVDYFKNPTITIEHK |
| | | QKEEVISEEKDLFKELEVRRNVIQHILNGDYKKDLKEEKQQDGDSEKV |
| | | KAFLDALLEFNYILNPFIIKDKNLRKEQEKDEEFYNEIKKLQESIFEAEIL |
| | | DLYNQTRNYITKKPYKLDKFKLTFGSGYFLSGWSNDMEEREGSILIKYN |
| | | EDRSKNYYLIIMAKPLTDDDKKQLFSDNGTHSKICIYEFQKMDMKNFPR |
| | | MFINSKGSNPAPAIEKYNLPIKTIWADYQKYKNLNQKGKDKFLEENPDF |
| | | RHNLIGYFKICAEKHESLAPFKHQFSSIWKPTKEYENLAQFYKDTLEAC |
| | | YNLKFENVNFDNISQLVSSGKLHLFKIHNKDFNPGSTGKKNLHTLYWE |
| | | MLFDEKNLQDVIFKLSGGAELFYREASILKNKIIHKIGEKVLKKFFKLPD |
| | | GKLEPVPAESIKNLSAYFRKELPEHELTEIDRKYIDNYSIIGKKDDKLGIM |
| | | KDERFTVDKIQFHCPITINFKSKNKNFINDDVLEYLHKRDDVHIIGLDRG |
| | | ERHLIYLTMINKDGKIVDNMQFSLNELQRRYKINGNEEIQKINYQKLLD |
| | | TREVSRTEARRNWQTIENIKNLKEGYLSLIVHQLAKLMIEKNAIVVMEN |
| | | LNYGFKDSRARVEKQIYQKFESILIKKLQYLVMDKNNLYDSGGVLSAY |
| | | QLTNQEVPAYKYISKQNGFLFYVPPDYTSKIDPETGFINLLDTRYYSRKN |
| | | AVALLNKFDKIYYDRDNKYFRFDFDYNSTDSNGNKNFDKLRVDISELT |
| | | RTKWSVCSHPAKRSITVQINNKWVRQPINDVTDKLIKLFEDKQIGYESG |
| | | KCLKDEILKVEDAKFFEDLLRYLSVLLALRHTYTENGVEYDLIISSVEKA |
| | | PGSNEFFVSGKDNNLPANADANGAYNIARKGLWLLRKLDEIDNQELAI |
| | | KKFNELKHAKEIKKNGEESKEDKGDRKRKKKWVSQWCPNKEWLAFA |
| | | QSMQDVSEK |
| SEQ | Cas12 | MNNGTNNFQNFIGISSLQKTLRNALIPTETTQQFIVKNGIIKEDELRGENR |
| ID | Variant | QILKDIMDDYYRGFISETLSSIDDIDWTSLFEKMEIQLKNGDNKDTLIKE |
| NO: | | QTEYRKAIHKKFANDDRFKNMFSAKLISDILPEFVIHNNNYSASEKEEK |
| 59 | | TQVIKLFSRFATSFKDYFKNRANCFSADDISSSSCHRIVNDNAEIFFSNAL |
| | | VYRRIVKSLSNDDINKISGDMKDSLKEMSLEEIYSYEKYGEFITQEGISF |
| | | YNDICGKVNSFMNLYCQKNKENKNLYKLQKLHKQILCIADTSYEVPYK |
| | | FESDEEVYQSVNGFLDNISSKHIVERLRKIGDNYNGYNLDKIYIVSKFYE |
| | | SVSQKTYRDWETINTALEIHYNNILPGNGKSKADKVKKAVKNDLQKSI |
| | | TEINELVSNYKLCSDDNIKAETYIHEISHILNNFEAQELKYNPEIHLVESE |
| | | LKASELKNVLDVIMNAFHWCSVFMTEELVDKDNNFYAELEEIYDEIYP |
| | | VISLYNLVRNYVTQKPYSTKKIKLNFGIPTLADGWSKSKEYSNNAIILM |
| | | RDNLYYLGIFNAKNKPDKKIIEGNTSENKGDYKKMIYNLLPGPNKMIPK |
| | | VFLSSKTGVETYKPSAYILEGYKQNKHIKSSKDFDITFCHDLIDYFKNCI |
| | | AIHPEWKNFGFDFSDTSTYEDISGFYREVELQGYKIDWTYISEKDIDLLQ |
| | | EKGQLYLFQIYNKDFSKKSTGNDNLHTMYLKNLFSEENLKDIVLKLNG |
| | | EAEIFFRKSSIKNPIIHKKGSILVNRTYEAEEKDQFGNIQIVRKNIPENIYQ |
| | | ELYKYFNDKSDKELSDEAAKLKNVVGHHEAATNIVKDYRYTYDKYFL |
| | | HMPITINFKANKTGFINDRILQYIAKEKDLHVIGIDRGERNLIYVSVIDTC |
| | | GNIVEQKSFNIVNGYDYQIKLKQQEGARQIARKEWKEIGKIKEIKEGYL |
| | | SLVIHEISKMVIKYNAIIAMEDLSYGFKKGRFKVERQVYQKFETMLINK |
| | | LNYLVFKDISITENGGLLKGYQLTYIPDKLKNVGHQCGCIFYVPAAYTS |
| | | KIDPTTGFVNIFKFKDLTVDAKREFIKKFDSIRYDSEKNLFCFTFDYNNFI |
| | | TQNTVMSKSSWSVYTYGVRIKRRFVNGRFSNESDTIDITKDMEKTLEM |
| | | TDINWRDGHDLRQDIIDYEIVQHIFEIFRLTVQMRNSLSELEDRDYDRLI |
| | | SPVLNENNIFYDSAKAGDALPKDADANGAYCIALKGLYEIKQITENWK |
| | | EDGKFSRDKLKISNKDWFDFIQNKRYL |
| SEQ | Cas12 | MSNLNTFISPEFTGKIKMTKSLKVSMIPIGETEHWIAKHKVFEKDRELFD |
| ID | Variant | KNLKARPILDEFIKYTVSRALPNLLFDFEAYYLVKKDRTKARAFEKELA |
| NO: | | KTVTDLILKEMDELKSASLIDSADFVKTTLKKFAGTHDIPGLSRIEAIESL |
| 60 | | EAASKLTALNGKFNTSRIAIINTLIPKRIIENFDIYLSNMEKIRNVYESGEF |
| | | GFLFERYPDTLLFMEPANYRTVCSPEAIEDYNRFISGYGDSTESWIKGFN |
| | | QELSEASNSSKSSNGGVRRYSLIKPLHKQHLFETKKFFTFASISSDDDVR |
| | | ELINSVKGSTEDACLNALAFFSSSDPKTLFVKGSYLHTLSAFLYGSANSY |
| | | ILPERIKEGEKARLTAEYDSVAKKTKAVTTRYNVAMNNISKKINEKIFSL |
| | | ADIDAYCCDISKRRSVREILLGIMQEMYAAVYGENGKWSNIEAEAVLD |
| | | SKTKIWKAKNGAVAKAVNDYLTAILEIRKFIRPFALRMEELEELGLDTS |
| | | SALDAGEITNTLFEAVRAQKLVHAYLTRNDADIALSTQVYFGGTQKAA |
| | | ASWWNYETGDIQNRQIALAKKDGMYYFIGTFDERGSYSIEPASPGEDY |
| | | YEMLDVKKGQDANKQIKKVLFSNKAIREHFADSSNDYVITTKVNSPITV |
| | | RREIFDKYQAGEFKLTSQKIRKGDLVGEKEMTYYREYMDLLFQMAKG |
| | | YTEYSRFNMDTLLPIEEYDTENDLLDDVNTNTIDYRWVRISAACIDDGV |
| | | RNGDIFVFRAQTSSMYGKRENKKGYTGLFLELVSDENLLVTRGMSLNS |
| | | AMSIYYRAKVHDAITVHKKGDVLVNKFTNARERIPENSYKAICAFYNS |
| | | GKSIEELTIEDRDWLAKATTRICSGEIIKDRRYTKNQYSISISYNINRSVN |
| | | NRKRVDLATIVDDTASAGRIISVTRGTKDLVYYTVIDDGGSVIEARSLN |
| | | VINGINYAKMLAQISEERHDSNANFDIPKRVETIKEAYCAFAVHEIISAA |
| | | LKHNALIVVELISDAIKDKYSLLDNQVFLKFENVLKNCLMSVKVKGAR |
| | | GMEPGSISNPLQLCNADDKSFRNGILYQIPSSYINICPVTGYADIIDYYNI |
| | | VSAGDIRNFFVRFENIVYNKEKARFEFSFDLKNIPIKLEKCPDRTKWTVL |
| | | GRGEITTYDPLTKSNHYVFDAAQMLAETVSKEGLDPCANIVEHIDELSA |
| | | ATLKKMFNTFRNIAKGIVSECDEVPVSYYKSPVIDEADIKNKSLDNKSIS |
| | | EIKCYNLDEKARYYLALAKSSSDGENKNRYVSSTAIEWLNYIQEKRTHE |

Alternatively, the Type V CRISPR/Cas enzyme is a programmable Cas14 nuclease. A Cas14 protein (this term is used interchangeably with the term “CasZ protein”, “Cas14”, “Cas14 polypeptide”, or “Cas14 protein”) of the present disclosure includes 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A naturally occurring Cas14 protein functions as an endonuclease that catalyzes cleavage at a specific sequence in a target nucleic acid. Catalytic residues of Cas14 include D405, E586 and D684 when numbered according to the amino acid sequence set forth in SEQ ID NO: 99. Thus, in some cases, the Cas14 protein has reduced activity and one or more of the above described amino acids (or one or more corresponding amino acids of any Cas14 protein) are mutated (e.g., substituted with an alanine).

Cas14 is short compared to previously identified CRISPR-Cas endonucleases, and thus use of this protein as an alternative provides the advantage that the nucleotide sequence encoding the protein is relatively short. This is useful, for example, in cases where a nucleic acid encoding the Cas14 protein is desirable, e.g., in situations that employ a viral vector (e.g., an AAV vector), for delivery to a cell such as a eukaryotic cell (e.g., mammalian cell, human cell, mouse cell, in vitro, ex vivo, in vivo) for research and/or clinical applications. In addition, in their natural context, the Cas14-encoding DNA sequences are present in loci that also have a Cas1 protein.

In some cases, a subject Cas14 protein has a length of 900 amino acids or less (e.g., 850 amino acids or less, 800 amino acids or less, 750 amino acids or less, or 700 amino acids or less). In some cases, a subject Cas14 protein has a length of 850 amino acids or less (e.g., 850 amino acids or less). In some cases, a subject Cas14 protein length of 800 amino acids or less (e.g., 750 amino acids or less). In some cases, a subject Cas14 protein has a length of 700 amino acids or less. In some cases, a subject Cas14 protein has a length of 650 amino acids or less. In some cases, a subject Cas14 protein has a length in a range of from 350-900 amino acids (e.g., 350-850, 350-800, 350-750, 350-700, 400-900, 400-850, 400-800, 400-750, or 400-700 amino acids).

A programmable Cas14 nuclease can be a Cas14a protein, a Cas14b protein, a Cas14c protein, a Cas14d protein, a Cas14e protein, a Cas14f protein, a Cas14g protein, a Cas14h protein, or a Cas14u protein. In some cases, a suitable Cas14 protein comprises an amino acid sequence having at least 80%, at least 85%, at least 90%, at least 95%, at least 98%, at least 99%, or 100%, amino acid sequence identity to any one of SEQ ID NO: 61-SEQ ID NO: 152.

TABLE 2
Cas14 Protein Sequences

| SEQ | |
|---|---|
| ID | |
| NO | Sequence |
| SEQ | MEVQKTVMKTLSLRILRPLYSQEIEKEIKEEKERRKQAGGTGELDGGFYKKLEKKHSE |
| ID | MFSFDRLNLLLNQLQREIAKVYNHAISELYIATIAQGNKSNKHYISSIVYNRAYGYFYN |
| NO: | AYIALGICSKVEANFRSNELLTQQSALPTAKSDNFPIVLHKQKGAEGEDGGFRISTEGS |
| 61 | DLIFEIPIPFYEYNGENRKEPYKWVKKGGQKPVLKLILSTFRRQRNKGWAKDEGTDAEI |
| | RKVTEGKYQVSQIEINRGKKLGEHQKWFANFSIEQPIYERKPNRSIVGGLDVGIRSPLV |
| | CAINNSFSRYSVDSNDVFKFSKQVFAFRRRLLSKNSLKRKGHGAAHKLEPITEMTEKN |
| | DKFRKKIIERWAKEVTNFFVKNQVGIVQIEDLSTMKDREDHFFNQYLRGFWPYYQMQ |
| | TLIENKLKEYGIEVKRVQAKYTSQLCSNPNCRYWNNYFNFEYRKVNKFPKFKCEKCN |
| | LEISADYNAARNLSTPDIEKFVAKATKGINLPEK |
| SEQ | MEEAKTVSKTLSLRILRPLYSAEIEKEIKEEKERRKQGGKSGELDSGFYKKLEKKHTQ |
| ID | MFGWDKLNLMLSQLQRQIARVFNQSISELYIETVIQGKKSNKHYTSKIVYNRAYSVFY |
| NO: | NAYLALGITSKVEANFRSTELLMQKSSLPTAKSDNFPILLHKQKGVEGEEGGFKISADG |
| 62 | NDLIFEIPIPFYEYDSANKKEPFKWIKKGGQKPTIKLILSTFRRQRNKGWAKDEGTDAEI |
| | RKVIEGKYQVSHIEINRGKKLGDHQKWFVNFTIEQPIYERKLDKNIIGGIDVGIKSPLVC |
| | AVNNSFARYSVDSNDVLKFSKQAFAFRRRLLSKNSLKRSGHGSKNKLDPITRMTEKN |
| | DRFRKKIIERWAKEVTNFFIKNQVGTVQIEDLSTMKDRQDNFFNQYLRGFWPYYQMQ |
| | NLIENKLKEYGIETKRIKARYTSQLCSNPSCRHWNSYFSFDHRKTNNFPKFKCEKCALE |
| | ISADYNAARNISTPDIEKFVAKATKGINLPDKNENVILE |
| SEQ | MAKNTITKTLKLRIVRPYNSAEVEKIVADEKNNREKIALEKNKDKVKEACSKHLKVA |
| ID | AYCTTQVERNACLFCKARKLDDKFYQKLRGQFPDAVFWQEISEIFRQLQKQAAEIYN |
| NO: | QSLIELYYEIFIKGKGIANASSVEHYLSDVCYTRAAELFKNAAIASGLRSKIKSNFRLKE |
| 63 | LKNMKSGLPTTKSDNFPIPLVKQKGGQYTGFEISNHNSDFIIKIPFGRWQVKKEIDKYRP |
| | WEKFDFEQVQKSPKPISLLLSTQRRKRNKGWSKDEGTEAEIKKVMNGDYQTSYIEVK |
| | RGSKIGEKSAWMLNLSIDVPKIDKGVDPSIIGGIDVGVKSPLVCAINNAFSRYSISDNDL |
| | FHFNKKMFARRRILLKKNRHKRAGHGAKNKLKPITILTEKSERFRKKLIERWACEIADF |
| | FIKNKVGTVQMENLESMKRKEDSYFNIRLRGFWPYAEMQNKIEFKLKQYGIEIRKVAP |
| | NNTSKTCSKCGHLNNYFNFEYRKKNKFPHFKCEKCNFKENADYNAALNISNPKLKST |
| | KEEP |
| SEQ | MERQKVPQIRKIVRVVPLRILRPKYSDVIENALKKFKEKGDDTNTNDFWRAIRDRDTE |
| ID | FFRKELNFSEDEINQLERDTLFRVGLDNRVLFSYFDFLQEKLMKDYNKIISKLFINRQSK |
| NO: | SSFENDLTDEEVEELIEKDVTPFYGAYIGKGIKSVIKSNLGGKFIKSVKIDRETKKVTKL |
| 64 | TAINIGLMGLPVAKSDTFPIKIIKTNPDYITFQKSTKENLQKIEDYETGIEYGDLLVQITIP |
| | WFKNENKDFSLIKTKEAIEYYKLNGVGKKDLLNINLVLTTYHIRKKKSWQIDGSSQSL |
| | VREMANGELEEKWKSFFDTFIKKYGDEGKSALVKRRVNKKSRAKGEKGRELNLDERI |
| | KRLYDSIKAKSFPSEINLIPENYKWKLHFSIEIPPMVNDIDSNLYGGIDFGEQNIATLCVK |
| | NIEKDDYDFLTIYGNDLLKHAQASYARRRIMRVQDEYKARGHGKSRKTKAQEDYSER |
| | MQKLRQKITERLVKQISDFFLWRNKFHMAVCSLRYEDLNTLYKGESVKAKRMRQFIN |
| | KQQLFNGIERKLKDYNSEIYVNSRYPHYTSRLCSKCGKLNLYFDFLKFRTKNIIIRKNPD |
| | GSEIKYMPFFICEFCGWKQAGDKNASANIADKDYQDKLNKEKEFCNIRKPKSKKEDIG |
| | EENEEERDYSRRFNRNSFIYNSLKKDNKLNQEKLFDEWKNQLKRKIDGRNKFEPKEYK |
| | DRFSYLFAYYQEIIKNESES |
| SEQ | MVPTELITKTLQLRVIRPLYFEEIEKELAELKEQKEKEFEETNSLLLESKKIDAKSLKKL |
| ID | KRKARSSAAVEFWKIAKEKYPDILTKPEMEFIFSEMQKMMARFYNKSMTNIFIEMNND |
| NO: | EKVNPLSLISKASTEANQVIKCSSISSGLNRKIAGSINKTKFKQVRDGLISLPTARTETFPI |
| 65 | SFYKSTANKDEIPISKINLPSEEEADLTITLPFPFFEIKKEKKGQKAYSYFNIIEKSGRSNN |
| | KIDLLLSTHRRQRRKGWKEEGGTSAEIRRLMEGEFDKEWEIYLGEAEKSEKAKNDLIK |
| | NMTRGKLSKDIKEQLEDIQVKYFSDNNVESWNDLSKEQKQELSKLRKKKVEELKDW |
| | KHVKEILKTRAKIGWVELKRGKRQRDRNKWFVNITITRPPFINKELDDTKFGGIDLGV |
| | KVPFVCAVHGSPARLIIKENEILQFNKMVSARNRQITKDSEQRKGRGKKNKFIKKEIFN |
| | ERNELFRKKIIERWANQIVKFFEDQKCATVQIENLESFDRTSYK |
| SEQ | MKSDTKDKKIIIHQTKTLSLRIVKPQSIPMEEFTDLVRYHQMIIFPVYNNGAIDLYKKLF |
| ID | KAKIQKGNEARAIKYFMNKIVYAPIANTVKNSYIALGYSTKMQSSFSGKRLWDLRFGE |
| NO: | ATPPTIKADFPLPFYNQSGFKVSSENGEFIIGIPFGQYTKKTVSDIEKKTSFAWDKFTLED |
| 66 | TTKKTLIELLLSTKTRKMNEGWKNNEGTEAEIKRVMDGTYQVTSLEILQRDDSWFVN |
| | FNIAYDSLKKQPDRDKIAGIHMGITRPLTAVIYNNKYRALSIYPNTVMHLTQKQLARIK |
| | EQRTNSKYATGGHGRNAKVTGTDTLSEAYRQRRKKIIEDWIASIVKFAINNEIGTIYLE |
| | DISNTNSFFAAREQKLIYLEDISNTNSFLSTYKYPISAISDTLQHKLEEKAIQVIRKKAYY |
| | VNQICSLCGHYNKGFTYQFRRKNKFPKMKCQGCLEATSTEFNAAANVANPDYEKLLI |
| | KHGLLQLKK |
| SEQ | MSTITRQVRLSPTPEQSRLLMAHCQQYISTVNVLVAAFDSEVLTGKVSTKDFRAALPS |
| ID | AVKNQALRDAQSVFKRSVELGCLPVLKKPHCQWNNQNWRVEGDQLILPICKDGKTQ |
| NO: | QERFRCAAVALEGKAGILRIKKKRGKWIADLTVTQEDAPESSGSAIMGVDLGIKVPAV |
| 67 | AHIGGKGTRFFGNGRSQRSMRRRFYARRKTLQKAKKLRAVRKSKGKEARWMKTINH |
| | QLSRQIVNHAHALGVGTIKIEALQGIRKGTTRKSRGAAARKNNRMTNTWSFSQLTLFI |
| | TYKAQRQGITVEQVDPAYTSQDCPACRARNGAQDRTYVCSECGWRGHRDTVGAINIS |
| | RRAGLSGHRRGATGA |
| SEQ | MIAQKTIKIKLNPTKEQIIKLNSIIEEYIKVSNFTAKKIAEIQESFTDSGLTQGTCSECGKE |
| ID | KTYRKYHLLKKDNKLFCITCYKRKYSQFTLQKVEFQNKTGLRNVAKLPKTYYTNAIR |
| NO: | FASDTFSGFDEIIKKKQNRLNSIQNRLNFWKELLYNPSNRNEIKIKVVKYAPKTDTREH |
| 68 | PHYYSEAEIKGRIKRLEKQLKKFKMPKYPEFTSETISLQRELYSWKNPDELKISSITDKN |
| | ESMNYYGKEYLKRYIDLINSQTPQILLEKENNSFYLCFPITKNIEMPKIDDTFEPVGIDW |
| | GITRNIAVVSILDSKTKKPKFVKFYSAGYILGKRKHYKSLRKHFGQKKRQDKINKLGT |
| | KEDRFIDSNIHKLAFLIVKEIRNHSNKPIILMENITDNREEAEKSMRQNILLHSVKSRLQN |
| | YIAYKALWNNIPTNLVKPEHTSQICNRCGHQDRENRPKGSKLFKCVKCNYMSNADFN |
| | ASINIARKFYIGEYEPFYKDNEKMKSGVNSISM |
| SEQ | LKLSEQENITTGVKFKLKLDKETSEGLNDYFDEYGKAINFAIKVIQKELAEDRFAGKVR |
| ID | LDENKKPLLNEDGKKIWDFPNEFCSCGKQVNRYVNGKSLCQECYKNKFTEYGIRKRM |
| NO: | YSAKGRKAEQDINIKNSTNKISKTHFNYAIREAFILDKSIKKQRKERFRRLREMKKKLQ |
| 69 | EFIEIRDGNKILCPKIEKQRVERYIHPSWINKEKKLEDFRGYSMSNVLGKIKILDRNIKRE |
| | EKSLKEKGQINFKARRLMLDKSVKFLNDNKISFTISKNLPKEYELDLPEKEKRLNWLK |
| | EKIKIIKNQKPKYAYLLRKDDNFYLQYTLETEFNLKEDYSGIVGIDRGVSHIAVYTFVH |
| | NNGKNERPLFLNSSEILRLKNLQKERDRFLRRKHNKKRKKSNMRNIEKKIQLILHNYS |
| | KQIVDFAKNKNAFIVFEKLEKPKKNRSKMSKKSQYKLSQFTFKKLSDLVDYKAKREGI |
| | KVLYISPEYTSKECSHCGEKVNTQRPFNGNSSLFKCNKCGVELNADYNASINIAKKGL |
| | NILNSTN |
| SEQ | MEESIITGVKFKLRIDKETTKKLNEYFDEYGKAINFAVKIIQKELADDRFAGKAKLDQN |
| ID | KNPILDENGKKIYEFPDEFCSCGKQVNKYVNNKPFCQECYKIRFTENGIRKRMYSAKG |
| NO: | RKAEHKINILNSTNKISKTHFNYAIREAFILDKSIKKQRKKRNERLRESKKRLQQFIDMR |
| 70 | DGKREICPTIKGQKVDRFIHPSWITKDKKLEDFRGYTLSIINSKIKILDRNIKREEKSLKE |
| | KGQIIFKAKRLMLDKSIRFVGDRKVLFTISKTLPKEYELDLPSKEKRLNWLKEKIEIIKN |
| | QKPKYAYLLRKNIESEKKPNYEYYLQYTLEIKPELKDFYDGAIGIDRGINHIAVCTFISN |
| | DGKVTPPKFFSSGEILRLKNLQKERDRFLLRKHNKNRKKGNMRVIENKINLILHRYSK |
| | QIVDMAKKLNASIVFEELGRIGKSRTKMKKSQRYKLSLFIFKKLSDLVDYKSRREGIRV |
| | TYVPPEYTSKECSHCGEKVNTQRPFNGNYSLFKCNKCGIQLNSDYNASINIAKKGLKIP |
| | NST |
| SEQ | LWTIVIGDFIEMPKQDLVTTGIKFKLDVDKETRKKLDDYFDEYGKAINFAVKIIQKNLK |
| ID | EDRFAGKIALGEDKKPLLDKDGKKIYNYPNESCSCGNQVRRYVNAKPFCVDCYKLKF |
| NO: | TENGIRKRMYSARGRKADSDINIKNSTNKISKTHFNYAIREGFILDKSLKKQRSKRIKKL |
| 71 | LELKRKLQEFIDIRQGQMVLCPKIKNQRVDKFIHPSWLKRDKKLEEFRGYSLSVVEGKI |
| | KIFNRNILREEDSLRQRGHVNFKANRIMLDKSVRFLDGGKVNFNLNKGLPKEYLLDLP |
| | KKENKLSWLNEKISLIKLQKPKYAYLLRREGSFFIQYTIENVPKTFSDYLGAIGIDRGIS |
| | HIAVCTFVSKNGVNKAPVFFSSGEILKLKSLQKQRDLFLRGKHNKIRKKSNMRNIDNKI |
| | NLILHKYSRNIVNLAKSEKAFIVFEKLEKIKKSRFKMSKSLQYKLSQFTFKKLSDLVEY |
| | KAKIEGIKVDYVPPEYTSKECSHCGEKVDTQRPFNGNSSLFKCNKCRVQLNADYNASI |
| | NIAKKSLNISN |
| SEQ | MSKTTISVKLKIIDLSSEKKEFLDNYFNEYAKATTFCQLRIRRLLRNTHWLGKKEKSSK |
| ID | KWIFESGICDLCGENKELVNEDRNSGEPAKICKRCYNGRYGNQMIRKLFVSTKKREVQ |
| NO: | ENMDIRRVAKLNNTHYHRIPEEAFDMIKAADTAEKRRKKNVEYDKKRQMEFIEMFND |
| 72 | EKKRAARPKKPNERETRYVHISKLESPSKGYTLNGIKRKIDGMGKKIERAEKGLSRKKI |
| | FGYQGNRIKLDSNWVRFDLAESEITIPSLFKEMKLRITGPTNVHSKSGQIYFAEWFERIN |
| | KQPNNYCYLIRKTSSNGKYEYYLQYTYEAEVEANKEYAGCLGVDIGCSKLAAAVYY |
| | DSKNKKAQKPIEIFTNPIKKIKMRREKLIKLLSRVKVRHRRRKLMQLSKTEPIIDYTCHK |
| | TARKIVEMANTAKAFISMENLETGIKQKQQARETKKQKFYRNMFLFRKLSKLIEYKAL |
| | LKGIKIVYVKPDYTSQTCSSCGADKEKTERPSQAIFRCLNPTCRYYQRDINADFNAAVN |
| | IAKKALNNTEVVTTLL |
| SEQ | MARAKNQPYQKLTTTTGIKFKLDLSEEEGKRFDEYFSEYAKAVNFCAKVIYQLRKNL |
| ID | KFAGKKELAAKEWKFEISNCDFCNKQKEIYYKNIANGQKVCKGCHRTNFSDNAIRKK |
| NO: | MIPVKGRKVESKFNIHNTTKKISGTHRHWAFEDAADIIESMDKQRKEKQKRLRREKRK |
| 73 | LSYFFELFGDPAKRYELPKVGKQRVPRYLHKIIDKDSLTKKRGYSLSYIKNKIKISERNI |
| | ERDEKSLRKASPIAFGARKIKMSKLDPKRAFDLENNVFKIPGKVIKGQYKFFGTNVAN |
| | EHGKKFYKDRISKILAGKPKYFYLLRKKVAESDGNPIFEYYVQWSIDTETPAITSYDNIL |
| | GIDAGITNLATTVLIPKNLSAEHCSHCGNNHVKPIFTKFFSGKELKAIKIKSRKQKYFLR |
| | GKHNKLVKIKRIRPIEQKVDGYCHVVSKQIVEMAKERNSCIALEKLEKPKKSKFRQRR |
| | REKYAVSMFVFKKLATFIKYKAAREGIEIIPVEPEGTSYTCSHCKNAQNNQRPYFKPNS |
| | KKSWTSMFKCGKCGIELNSDYNAAFNIAQKALNMTSA |
| SEQ | MDEKHFFCSYCNKELKISKNLINKISKGSIREDEAVSKAISIHNKKEHSLILGIKFKLFIEN |
| ID | KLDKKKLNEYFDNYSKAVTFAARIFDKIRSPYKFIGLKDKNTKKWTFPKAKCVFCLEE |
| NO: | KEVAYANEKDNSKICTECYLKEFGENGIRKKIYSTRGRKVEPKYNIFNSTKELSSTHYN |
| 74 | YAIRDAFQLLDALKKQRQKKLKSIFNQKLRLKEFEDIFSDPQKRIELSLKPHQREKRYIH |
| | LSKSGQESINRGYTLRFVRGKIKSLTRNIEREEKSLRKKTPIHFKGNRLMIFPAGIKFDFA |
| | SNKVKISISKNLPNEFNFSGTNVKNEHGKSFFKSRIELIKTQKPKYAYVLRKIKREYSKL |
| | RNYEIEKIRLENPNADLCDFYLQYTIETESRNNEEINGIIGIDRGITNLACLVLLKKGDKK |
| | PSGVKFYKGNKILGMKIAYRKHLYLLKGKRNKLRKQRQIRAIEPKINLILHQISKDIVKI |
| | AKEKNFAIALEQLEKPKKARFAQRKKEKYKLALFTFKNLSTLIEYKSKREGIPVIYVPP |
| | EKTSQMCSHCAINGDEHVDTQRPYKKPNAQKPSYSLFKCNKCGIELNADYNAAFNIA |
| | QKGLKTLMLNHSH |
| SEQ | MLQTLLVKLDPSKEQYKMLYETMERFNEACNQIAETVFAIHSANKIEVQKTVYYPIRE |
| ID | KFGLSAQLTILAIRKVCEAYKRDKSIKPEFRLDGALVYDQRVLSWKGLDKVSLVTLQG |
| NO: | RQIIPIKFGDYQKARMDRIRGQADLILVKGVFYLCVVVEVSEESPYDPKGVLGVDLGIK |
| 75 | NLAVDSDGEVHSGEQTTNTRERLDSLKARLQSKGTKSAKRHLKKLSGRMAKFSKDV |
| | NHCISKKLVAKAKGTLMSIALEDLQGIRDRVTVRKAQRRNLHTWNFGLLRMFVDYK |
| | AKIAGVPLVFVDPRNTSRTCPSCGHVAKANRPTRDEFRCVSCGFAGAADHIAAMNIAF |
| | RAEVSQPIVTRFFVQSQAPSFRVG |
| SEQ | MDEEPDSAEPNLAPISVKLKLVKLDGEKLAALNDYFNEYAKAVNFCELKMQKIRKNL |
| ID | VNIRGTYLKEKKAWINQTGECCICKKIDELRCEDKNPDINGKICKKCYNGRYGNQMIR |
| NO: | KLFVSTNKRAVPKSLDIRKVARLHNTHYHRIPPEAADIIKAIETAERKRRNRILFDERRY |
| 76 | NELKDALENEEKRVARPKKPKEREVRYVPISKKDTPSKGYTMNALVRKVSGMAKKIE |
| | RAKRNLNKRKKIEYLGRRILLDKNWVRFDFDKSEISIPTMKEFFGEMRFEITGPSNVMS |
| | PNGREYFTKWFDRIKAQPDNYCYLLRKESEDETDFYLQYTWRPDAHPKKDYTGCLGI |
| | DIGGSKLASAVYFDADKNRAKQPIQIFSNPIGKWKTKRQKVIKVLSKAAVRHKTKKLE |
| | SLRNIEPRIDVHCHRIARKIVGMALAANAFISMENLEGGIREKQKAKETKKQKFSRNMF |
| | VFRKLSKLIEYKALMEGVKVVYIVPDYTSQLCSSCGTNNTKRPKQAIFMCQNTECRYF |
| | GKNINADFNAAINIAKKALNRKDIVRELS |
| SEQ | MEKNNSEQTSITTGIKFKLKLDKETKEKLNNYFDEYGKAINFAVRIIQMQLNDDRLAG |
| ID | KYKRDEKGKPILGEDGKKILEIPNDFCSCGNQVNHYVNGVSFCQECYKKRFSENGIRK |
| NO: | RMYSAKGRKAEQDINIKNSTNKISKTHFNYAIREAFNLDKSIKKQREKRFKKLKDMKR |
| 77 | KLQEFLEIRDGKRVICPKIEKQKVERYIHPSWINKEKKLEEFRGYSLSIVNSKIKSFDRNI |
| | QREEKSLKEKGQINFKAQRLMLDKSVKFLKDNKVSFTISKELPKTFELDLPKKEKKLN |
| | WLNEKLEIIKNQKPKYAYLLRKENNIFLQYTLDSIPEIHSEYSGAVGIDRGVSHIAVYTF |
| | LDKDGKNERPFFLSSSGILRLKNLQKERDKFLRKKHNKIRKKGNMRNIEQKINLILHEY |
| | SKQIVNFAKDKNAFIVFELLEKPKKSRERMSKKIQYKLSQFTFKKLSDLVDYKAKREGI |
| | KVIYVEPAYTSKDCSHCGERVNTQRPFNGNFSLFKCNKCGIVLNSDYNASLNIARKGL |
| | NISAN |
| SEQ | MAEEKFFFCEKCNKDIKIPKNYINKQGAEEKARAKHEHRVHALILGIKFKIYPKKEDIS |
| ID | KLNDYFDEYAKAVTFTAKIVDKLKAPFLFAGKRDKDTSKKKWVFPVDKCSFCKEKTE |
| NO: | INYRTKQGKNICNSCYLTEFGEQGLLEKIYATKGRKVSSSFNLFNSTKKLTGTHNNYV |
| 78 | VKESLQLLDALKKQRSKRLKKLSNTRRKLKQFEEMFEKEDKRFQLPLKEKQRELRFIH |
| | VSQKDRATEFKGYTMNKIKSKIKVLRRNIEREQRSLNRKSPVFFRGTRIRLSPSVQFDD |
| | KDNKIKLTLSKELPKEYSFSGLNVANEHGRKFFAEKLKLIKENKSKYAYLLRRQVNKN |
| | NKKPIYDYYLQYTVEFLPNIITNYNGILGIDRGINTLACIVLLENKKEKPSFVKFFSGKGI |
| | LNLKNKRRKQLYFLKGVHNKYRKQQKIRPIEPRIDQILHDISKQIIDLAKEKRVAISLEQ |
| | LEKPQKPKFRQSRKAKYKLSQFNFKTLSNYIDYKAKKEGIRVIYIAPEMTSQNCSRCA |
| | MKNDLHVNTQRPYKNTSSLFKCNKCGVELNADYNAAFNIAQKGLKILNS |
| SEQ | MISLKLKLLPDEEQKKLLDEMFWKWASICTRVGFGRADKEDLKPPKDAEGVWFSLTQ |
| ID | LNQANTDINDLREAMKHQKHRLEYEKNRLEAQRDDTQDALKNPDRREISTKRKDLFR |
| NO: | PKASVEKGFLKLKYHQERYWVRRLKEINKLIERKTKTLIKIEKGRIKFKATRITLHQGSF |
| 79 | KIRFGDKPAFLIKALSGKNQIDAPFVVVPEQPICGSVVNSKKYLDEITTNFLAYSVNAM |
| | LFGLSRSEEMLLKAKRPEKIKKKEEKLAKKQSAFENKKKELQKLLGRELTQQEEAIIEE |
| | TRNQFFQDFEVKITKQYSELLSKIANELKQKNDFLKVNKYPILLRKPLKKAKSKKINNL |
| | SPSEWKYYLQFGVKPLLKQKSRRKSRNVLGIDRGLKHLLAVTVLEPDKKTFVWNKLY |
| | PNPITGWKWRRRKLLRSLKRLKRRIKSQKHETIHENQTRKKLKSLQGRIDDLLHNISRK |
| | IVETAKEYDAVIVVEDLQSMRQHGRSKGNRLKTLNYALSLFDYANVMQLIKYKAGIE |
| | GIQIYDVKPAGTSQNCAYCLLAQRDSHEYKRSQENSKIGVCLNPNCQNHKKQIDADLN |
| | AARVIASCYALKINDSQPFGTRKRFKKRTTN |
| SEQ | METLSLKLKLNPSKEQLLVLDKMFWKWASICTRLGLKKAEMSDLEPPKDAEGVWFS |
| ID | KTQLNQANTDVNDLRKAMQHQGKRIEYELDKVENRRNEIQEMLEKPDRRDISPNRKD |
| NO: | LFRPKAAVEKGYLKLKYHKLGYWSKELKTANKLIERKRKTLAKIDAGKMKFKPTRIS |
| 80 | LHTNSFRIKFGEEPKIALSTTSKHEKIELPLITSLQRPLKTSCAKKSKTYLDAAILNFLAY |
| | STNAALFGLSRSEEMLLKAKKPEKIEKRDRKLATKRESFDKKLKTLEKLLERKLSEKE |
| | KSVFKRKQTEFFDKFCITLDETYVEALHRIAEELVSKNKYLEIKKYPVLLRKPESRLRS |
| | KKLKNLKPEDWTYYIQFGFQPLLDTPKPIKTKTVLGIDRGVRHLLAVSIFDPRTKTFTF |
| | NRLYSNPIVDWKWRRRKLLRSIKRLKRRLKSEKHVHLHENQFKAKLRSLEGRIEDHFH |
| | NLSKEIVDLAKENNSVIVVENLGGMRQHGRGRGKWLKALNYALSHFDYAKVMQLIK |
| | YKAELAGVFVYDVAPAGTSINCAYCLLNDKDASNYTRGKVINGKKNTKIGECKTCKK |
| | EFDADLNAARVIALCYEKRLNDPQPFGTRKQFKPKKP |
| SEQ | MKALKLQLIPTRKQYKILDEMFWKWASLANRVSQKGESKETLAPKKDIQKIQFNATQ |
| ID | LNQIEKDIKDLRGAMKEQQKQKERLLLQIQERRSTISEMLNDDNNKERDPHRPLNFRP |
| NO: | KGWRKFHTSKHWVGELSKILRQEDRVKKTIERIVAGKISFKPKRIGIWSSNYKINFFKR |
| 81 | KISINPLNSKGFELTLMTEPTQDLIGKNGGKSVLNNKRYLDDSIKSLLMFALHSRFFGL |
| | NNTDTYLLGGKINPSLVKYYKKNQDMGEFGREIVEKFERKLKQEINEQQKKIIMSQIKE |
| | QYSNRDSAFNKDYLGLINEFSEVFNQRKSERAEYLLDSFEDKIKQIKQEIGESLNISDW |
| | DFLIDEAKKAYGYEEGFTEYVYSKRYLEILNKIVKAVLITDIYFDLRKYPILLRKPLDKI |
| | KKISNLKPDEWSYYIQFGYDSINPVQLMSTDKFLGIDRGLTHLLAYSVFDKEKKEFIIN |
| | QLEPNPIMGWKWKLRKVKRSLQHLERRIRAQKMVKLPENQMKKKLKSIEPKIEVHYH |
| | NISRKIVNLAKDYNASIVVESLEGGGLKQHGRKKNARNRSLNYALSLFDYGKIASLIK |
| | YKADLEGVPMYEVLPAYTSQQCAKCVLEKGSFVDPEIIGYVEDIGIKGSLLDSLFEGTE |
| | LSSIQVLKKIKNKIELSARDNHNKEINLILKYNFKGLVIVRGQDKEEIAEHPIKEINGKFA |
| | ILDFVYKRGKEKVGKKGNQKVRYTGNKKVGYCSKHGQVDADLNASRVIALCKYLDI |
| | NDPILFGEQRKSFK |
| SEQ | MVTRAIKLKLDPTKNQYKLLNEMFWKWASLANRFSQKGASKETLAPKDGTQKIQFN |
| ID | ATQLNQIKKDVDDLRGAMEKQGKQKERLLIQIQERLLTISEILRDDSKKEKDPHRPQNF |
| NO: | RPFGWRRFHTSAYWSSEASKLTRQVDRVRRTIERIKAGKINFKPKRIGLWSSTYKINFL |
| 82 | KKKINISPLKSKSFELDLITEPQQKIIGKEGGKSVANSKKYLDDSIKSLLIFAIKSRLFGLN |
| | NKDKPLFENIITPNLVRYHKKGQEQENFKKEVIKKFENKLKKEISQKQKEIIFSQIERQY |
| | ENRDATFSEDYLRAISEFSEIFNQRKKERAKELLNSFNEKIRQLKKEVNGNISEEDLKIL |
| | EVEAEKAYNYENGFIEWEYSEQFLGVLEKIARAVLISDNYFDLKKYPILIRKPTNKSKKI |
| | TNLKPEEWDYYIQFGYGLINSPMKIETKNFMGIDRGLTHLLAYSIFDRDSEKFTINQLEL |
| | NPIKGWKWKLRKVKRSLQHLERRMRAQKGVKLPENQMKKRLKSIEPKIESYYHNLSR |
| | KIVNLAKANNASIVVESLEGGGLKQHGRKKNSRHRALNYALSLFDYGKIASLIKYKSD |
| | LEGVPMYEVLPAYTSQQCAKCVLKKGSFVEPELIGYIEEIGFKENLLTLLFEDTGLSSVQ |
| | VLKKSKNKMTLSARDKEGKMVDLVLKYNFKGLVISQEKKKEEIVEFPIKEIDGKFAVL |
| | DSAYKRGKERISKKGNQKLVYTGNKKVGYCSVHGQVDADLNASRVIALCKYLGINEP |
| | IVFGEQRKSFK |
| SEQ | LDLITEPIQPHKSSSLRSKEFLEYQISDFLNFSLHSLFFGLASNEGPLVDFKIYDKIVIPKP |
| ID | EERFPKKESEEGKKLDSFDKRVEEYYSDKLEKKIERKLNTEEKNVIDREKTRIWGEVN |
| NO: | KLEEIRSIIDEINEIKKQKHISEKSKLLGEKWKKVNNIQETLLSQEYVSLISNLSDELTNK |
| 83 | KKELLAKKYSKFDDKIKKIKEDYGLEFDENTIKKEGEKAFLNPDKFSKYQFSSSYLKLI |
| | GEIARSLITYKGFLDLNKYPIIFRKPINKVKKIHNLEPDEWKYYIQFGYEQINNPKLETE |
| | NILGIDRGLTHILAYSVFEPRSSKFILNKLEPNPIEGWKWKLRKLRRSIQNLERRWRAQ |
| | DNVKLPENQMKKNLRSIEDKVENLYHNLSRKIVDLAKEKNACIVFEKLEGQGMKQHG |
| | RKKSDRLRGLNYKLSLFDYGKIAKLIKYKAEIEGIPIYRIDSAYTSQNCAKCVLESRRFA |
| | QPEEISCLDDFKEGDNLDKRILEGTGLVEAKIYKKLLKEKKEDFEIEEDIAMFDTKKVIK |
| | ENKEKTVILDYVYTRRKEIIGTNHKKNIKGIAKYTGNTKIGYCMKHGQVDADLNASRT |
| | IALCKNFDINNPEIWK |
| SEQ | MSDESLVSSEDKLAIKIKIVPNAEQAKMLDEMFKKWSSICNRISRGKEDIETLRPDEGK |
| ID | ELQFNSTQLNSATMDVSDLKKAMARQGERLEAEVSKLRGRYETIDASLRDPSRRHTN |
| NO: | PQKPSSFYPSDWDISGRLTPRFHTARHYSTELRKLKAKEDKMLKTINKIKNGKIVFKPK |
| 84 | RITLWPSSVNMAFKGSRLLLKPFANGFEMELPIVISPQKTADGKSQKASAEYMRNALL |
| | GLAGYSINQLLFGMNRSQKMLANAKKPEKVEKFLEQMKNKDANFDKKIKALEGKWL |
| | LDRKLKESEKSSIAVVRTKFFKSGKVELNEDYLKLLKHMANEILERDGFVNLNKYPILS |
| | RKPMKRYKQKNIDNLKPNMWKYYIQFGYEPIFERKASGKPKNIMGIDRGLTHLLAVA |
| | VFSPDQQKFLFNHLESNPIMHWKWKLRKIRRSIQHMERRIRAEKNKHIHEAQLKKRLG |
| | SIEEKTEQHYHIVSSKIINWAIEYEAAIVLESLSHMKQRGGKKSVRTRALNYALSLFDY |
| | EKVARLITYKARIRGIPVYDVLPGMTSKTCATCLLNGSQGAYVRGLETTKAAGKATK |
| | RKNMKIGKCMVCNSSENSMIDADLNAARVIAICKYKNLNDPQPAGSRKVFKRF |
| SEQ | MLALKLKIMPTEKQAEILDAMFWKWASICSRIAKMKKKVSVKENKKELSKKIPSNSDI |
| ID | WFSKTQLCQAEVDVGDHKKALKNFEKRQESLLDELKYKVKAINEVINDESKREIDPN |
| NO: | NPSKFRIKDSTKKGNLNSPKFFTLKKWQKILQENEKRIKKKESTIEKLKRGNIFFNPTKI |
| 85 | SLHEEEYSINFGSSKLLLNCFYKYNKKSGINSDQLENKFNEFQNGLNIICSPLQPIRGSSK |
| | RSFEFIRNSIINFLMYSLYAKLFGIPRSVKALMKSNKDENKLKLEEKLKKKKSSFNKTV |
| | KEFEKMIGRKLSDNESKILNDESKKFFEIIKSNNKYIPSEEYLKLLKDISEEIYNSNIDFKP |
| | YKYSILIRKPLSKFKSKKLYNLKPTDYKYYLQLSYEPFSKQLIATKTILGIDRGLKHLLA |
| | VSVFDPSQNKFVYNKLIKNPVFKWKKRYHDLKRSIRNRERRIRALTGVHIHENQLIKK |
| | LKSMKNKINVLYHNVSKNIVDLAKKYESTIVLERLENLKQHGRSKGKRYKKLNYVLS |
| | NFDYKKIESLISYKAKKEGVPVSNINPKYTSKTCAKCLLEVNQLSELKNEYNRDSKNS |
| | KIGICNIHGQIDADLNAARVIALCYSKNLNEPHFK |
| SEQ | VINLFGYKFALYPNKTQEELLNKHLGECGWLYNKAIEQNEYYKADSNIEEAQKKFELL |
| ID | PDKNSDEAKVLRGNISKDNYVYRTLVKKKKSEINVQIRKAVVLRPAETIRNLAKVKKK |
| NO: | GLSVGRLKFIPIREWDVLPFKQSDQIRLEENYLILEPYGRLKFKMHRPLLGKPKTFCIKR |
| 86 | TATDRWTISFSTEYDDSNMRKNDGGQVGIDVGLKTHLRLSNENPDEDPRYPNPKIWK |
| | RYDRRLTILQRRISKSKKLGKNRTRLRLRLSRLWEKIRNSRADLIQNETYEILSENKLIAI |
| | EDLNVKGMQEKKDKKGRKGRTRAQEKGLHRSISDAAFSEFRRVLEYKAKRFGSEVKP |
| | VSAIDSSKECHNCGNKKGMPLESRIYECPKCGLKIDRDLNSAKVILARATGVRPGSNA |
| | RADTKISATAGASVQTEGTVSEDFRQQMETSDQKPMQGEGSKEPPMNPEHKSSGRGS |
| | KHVNIGCKNKVGLYNEDENSRSTEKQIMDENRSTTEDMVEIGALHSPVLTT |
| SEQ | MIASIDYEAVSQALIVFEFKAKGKDSQYQAIDEAIRSYRFIRNSCLRYWMDNKKVGKY |
| ID | DLNKYCKVLAKQYPFANKLNSQARQSAAECSWSAISRFYDNCKRKVSGKKGFPKFK |
| NO: | KHARSVEYKTSGWKLSENRKAITFTDKNGIGKLKLKGTYDLHFSQLEDMKRVRLVRR |
| 87 | ADGYYVQFCISVDVKVETEPTGKAIGLDVGIKYFLADSSGNTIENPQFYRKAEKKLNR |
| | ANRRKSKKYIRGVKPQSKNYHKARCRYARKHLRVSRQRKEYCKRVAYCVIHSNDVV |
| | AYEDLNVKGMVKNRHLAKSISDVAWSTFRHWLEYFAIKYGKLTIPVAPHNTSQNCSN |
| | CDKKVPKSLSTRTHICHHCGYSEDRDVNAAKNILKKALSTVGQTGSLKLGEIEPLLVLE |
| | QSCTRKFDL |
| SEQ | LAEENTLHLTLAMSLPLNDLPENRTRSELWRRQWLPQKKLSLLLGVNQSVRKAAADC |
| NO: | LRWFEPYQELLWWEPTDPDGKKLLDKEGRPIKRTAGHMRVLRKLEEIAPFRGYQLGS |
| 88 | AVKNGLRHKVADLLLSYAKRKLDPQFTDKTSYPSIGDQFPIVWTGAFVCYEQSITGQL |
| | YLYLPLFPRGSHQEDITNNYDPDRGPALQVFGEKEIARLSRSTSGLLLPLQFDKWGEAT |
| | FIRGENNPPTWKATHRRSDKKWLSEVLLREKDFQPKRVELLVRNGRIFVNVACEIPTK |
| | PLLEVENFMGVSFGLEHLVTVVVINRDGNVVHQRQEPARRYEKTYFARLERLRRRGG |
| | PFSQELETFHYRQVAQIVEEALRFKSVPAVEQVGNIPKGRYNPRLNLRLSYWPFGKLA |
| | DLTSYKAVKEGLPKPYSVYSATAKMLCSTCGAANKEGDQPISLKGPTVYCGNCGTRH |
| | NTGFNTALNLARRAQELFVKGVVAR |
| SEQ | MSQSLLKWHDMAGRDKDASRSLQKSAVEGVLLHLTASHRVALEMLEKSVSQTVAVT |
| ID | MEAAQQRLVIVLEDDPTKATSRKRVISADLQFTREEFGSLPNWAQKLASTCPEIATKY |
| NO: | ADKHINSIRIAWGVAKESTNGDAVEQKLQWQIRLLDVTMFLQQLVLQLADKALLEQIP |
| 89 | SSIRGGIGQEVAQQVTSHIQLLDSGTVLKAELPTISDRNSELARKQWEDAIQTVCTYAL |
| | PFSRERARILDPGKYAAEDPRGDRLINIDPMWARVLKGPTVKSLPLLFVSGSSIRIVKLT |
| | LPRKHAAGHKHTFTATYLVLPVSREWINSLPGTVQEKVQWWKKPDVLATQELLVGK |
| | GALKKSANTLVIPISAGKKRFFNHILPALQRGFPLQWQRIVGRSYRRPATHRKWFAQL |
| | TIGYTNPSSLPEMALGIHFGMKDILWWALADKQGNILKDGSIPGNSILDFSLQEKGKIE |
| | RQQKAGKNVAGKKYGKSLLNATYRVVNGVLEFSKGISAEHASQPIGLGLETIRFVDK |
| | ASGSSPVNARHSNWNYGQLSGIFANKAGPAGFSVTEITLKKAQRDLSDAEQARVLAIE |
| | ATKRFASRIKRLATKRKDDTLFV |
| SEQ | VEPVEKERFYYRTYTFRLDGQPRTQNLTTQSGWGLLTKAVLDNTKHYWEIVHHARIA |
| ID | NQPIVFENPVIDEQGNPKLNKLGQPRFWKRPISDIVNQLRALFENQNPYQLGSSLIQGT |
| NO: | YWDVAENLASWYALNKEYLAGTATWGEPSFPEPHPLTEINQWMPLTFSSGKVVRLLK |
| 90 | NASGRYFIGLPILGENNPCYRMRTIEKLIPCDGKGRVTSGSLILFPLVGIYAQQHRRMTD |
| | ICESIRTEKGKLAWAQVSIDYVREVDKRRRMRRTRKSQGWIQGPWQEVFILRLVLAH |
| | KAPKLYKPRCFAGISLGPKTLASCVILDQDERVVEKQQWSGSELLSLIHQGEERLRSLR |
| | EQSKPTWNAAYRKQLKSLINTQVFTIVTFLRERGAAVRLESIARVRKSTPAPPVNFLLS |
| | HWAYRQITERLKDLAIRNGMPLTHSNGSYGVRFTCSQCGATNQGIKDPTKYKVDIESE |
| | TFLCSICSHREIAAVNTATNLAKQLLDE |
| SEQ | MNDTETSETLTSHRTVCAHLHVVGETGSLPRLVEAALAELITLNGRATQALLSLAKNG |
| ID | LVLRRDKEENLIAAELTLPCRKNKYADVAAKAGEPILATRINNKGKLVTKKWYGEGN |
| NO: | SYHIVRFTPETGMFTVRVFDRYAFDEELLHLHSEVVFGSDLPKGIKAKTDSLPANFLQA |
| 91 | VFTSFLELPFQGFPDIVVKPAMKQAAEQLLSYVQLEAGENQQAEYPDTNERDPELRLV |
| | EWQKSLHELSVRTEPFEFVRARDIDYYAETDRRGNRFVNITPEWTKFAESPFARRLPLK |
| | IPPEFCILLRRKTEGHAKIPNRIYLGLQIFDGVTPDSTLGVLATAEDGKLFWWHDHLDE |
| | FSNLEGKPEPKLKNKPQLLMVSLEYDREQRFEESVGGDRKICLVTLKETRNFRRGWNG |
| | RILGIHFQHNPVITWALMDHDAEVLEKGFIEGNAFLGKALDKQALNEYLQKGGKWVG |
| | DRSFGNKLKGITHTLASLIVRLAREKDAWIALEEISWVQKQSADSVANHEIVEQPHHSL |
| | TR |
| SEQ | MNDTETSETLTSHRTVCAHLHVVGETGSLPRLVEAALAELITLNGRATQALLSLAKNG |
| ID | LVLRRDKEENLIAAELTLPCRKNKYADVAAKAGEPILATRINNKGKLVTKKWYGEGN |
| NO: | SYHIVRFTPETGMFTVRVFDRYAFDEELLHLHSEVVFGSDLPKGIKAKTDSLPANFLQA |
| 92 | VFTSFLELPFQGFPDIVVKPAMKQAAEQLLSYVQLEAGENQQAEYPDTNERDPELRLV |
| | EWQKSLHELSVRTEPFEFVRARDIDYYAETDRRGNRFVNITPEWTKFAESPFARRLPLK |
| | IPPEFCILLRRKTEGHAKIPNRIYLGLQIFDGVTPDSTLGVLATAEDGKLFWWHDHLDE |
| | FSNLEGKPEPKLKNKPQLLMVSLEYDREQRFEESVGGDRKICLVTLKETRNFRRGRHG |
| | HTRTDRLPAGNTLWRADFATSAEVAAPKWNGRILGIHFQHNPVITWALMDHDAEVLE |
| | KGFIEGNAFLGKALDKQALNEYLQKGGKWVGDRSFGNKLKGITHTLASLIVRLAREK |
| | DAWIALEEISWVQKQSADSVANRRFSMWNYSRLATLIEWLGTDIATRDCGTAAPLAH |
| | KVSDYLTHFTCPECGACRKAGQKKEIADTVRAGDILTCRKCGFSGPIPDNFIAEFVAKK |
| | ALERMLKKKPV |
| SEQ | MAKRNFGEKSEALYRAVRFEVRPSKEELSILLAVSEVLRMLFNSALAERQQVFTEFIAS |
| ID | LYAELKSASVPEEISEIRKKLREAYKEHSISLFDQINALTARRVEDEAFASVTRNWQEET |
| NO: | LDALDGAYKSFLSLRRKGDYDAHSPRSRDSGFFQKIPGRSGFKIGEGRIALSCGAGRKL |
| 93 | SFPIPDYQQGRLAETTKLKKFELYRDQPNLAKSGRFWISVVYELPKPEATTCQSEQVAF |
| | VALGASSIGVVSQRGEEVIALWRSDKHWVPKIEAVEERMKRRVKGSRGWLRLLNSGK |
| | RRMHMISSRQHVQDEREIVDYLVRNHGSHFVVTELVVRSKEGKLADSSKPERGGSLG |
| | LNWAAQNTGSLSRLVRQLEEKVKEHGGSVRKHKLTLTEAPPARGAENKLWMARKLR |
| | ESFLKEV |
| SEQ | LAKNDEKELLYQSVKFEIYPDESKIRVLTRVSNILVLVWNSALGERRARFELYIAPLYE |
| ID | ELKKFPRKSAESNALRQKIREGYKEHIPTFFDQLKKLLTPMRKEDPALLGSVPRAYQEE |
| NO: | TLNTLNGSFVSFMTLRRNNDMDAKPPKGRAEDRFHEISGRSGFKIDGSEFVLSTKEQK |
| 94 | LRFPIPNYQLEKLKEAKQIKKFTLYQSRDRRFWISIAYEIELPDQRPFNPEEVIYIAFGAS |
| | SIGVISPEGEKVIDFWRPDKHWKPKIKEVENRMRSCKKGSRAWKKRAAARRKMYAM |
| | TQRQQKLNHREIVASLLRLGFHFVVTEYTVRSKPGKLADGSNPKRGGAPQGFNWSAQ |
| | NTGSFGEFILWLKQKVKEQGGTVQTFRLVLGQSERPEKRGRDNKIEMVRLLREKYLES |
| | QTIVV |
| SEQ | MAKGKKKEGKPLYRAVRFEIFPTSDQITLFLRVSKNLQQVWNEAWQERQSCYEQFFG |
| ID | SIYERIGQAKKRAQEAGFSEVWENEAKKGLNKKLRQQEISMQLVSEKESLLQELSIAF |
| NO: | QEHGVTLYDQINGLTARRIIGEFALIPRNWQEETLDSLDGSFKSFLALRKNGDPDAKPP |
| 95 | RQRVSENSFYKIPGRSGFKVSNGQIYLSFGKIGQTLTSVIPEFQLKRLETAIKLKKFELCR |
| | DERDMAKPGRFWISVAYEIPKPEKVPVVSKQITYLAIGASRLGVVSPKGEFCLNLPRSD |
| | YHWKPQINALQERLEGVVKGSRKWKKRMAACTRMFAKLGHQQKQHGQYEVVKKL |
| | LRHGVHFVVTELKVRSKPGALADASKSDRKGSPTGPNWSAQNTGNIARLIQKLTDKA |
| | SEHGGTVIKRNPPLLSLEERQLPDAQRKIFIAKKLREEFLADQK |
| SEQ | MAKREKKDDVVLRGTKMRIYPTDRQVTLMDMWRRRCISLWNLLLNLETAAYGAKN |
| ID | TRSKLGWRSIWARVVEENHAKALIVYQHGKCKKDGSFVLKRDGTVKHPPRERFPGDR |
| NO: | KILLGLFDALRHTLDKGAKCKCNVNQPYALTRAWLDETGHGARTADIIAWLKDFKGE |
| 96 | CDCTAISTAAKYCPAPPTAELLTKIKRAAPADDLPVDQAILLDLFGALRGGLKQKECD |
| | HTHARTVAYFEKHELAGRAEDILAWLIAHGGTCDCKIVEEAANHCPGPRLFIWEHELA |
| | MIMARLKAEPRTEWIGDLPSHAAQTVVKDLVKALQTMLKERAKAAAGDESARKTGF |
| | PKFKKQAYAAGSVYFPNTTMFFDVAAGRVQLPNGCGSMRCEIPRQLVAELLERNLKP |
| | GLVIGAQLGLLGGRIWRQGDRWYLSCQWERPQPTLLPKTGRTAGVKIAASIVFTTYDN |
| | RGQTKEYPMPPADKKLTAVHLVAGKQNSRALEAQKEKEKKLKARKERLRLGKLEKG |
| | HDPNALKPLKRPRVRRSKLFYKSAARLAACEAIERDRRDGFLHRVTNEIVHKFDAVSV |
| | QKMSVAPMMRRQKQKEKQIESKKNEAKKEDNGAAKKPRNLKPVRKLLRHVAMARG |
| | RQFLEYKYNDLRGPGSVLIADRLEPEVQECSRCGTKNPQMKDGRRLLRCIGVLPDGTD |
| | CDAVLPRNRNAARNAEKRLRKHREAHNA |
| SEQ | MNEVLPIPAVGEDAADTIMRGSKMRIYPSVRQAATMDLWRRRCIQLWNLLLELEQAA |
| ID | YSGENRRTQIGWRSIWATVVEDSHAEAVRVAREGKKRKDGTFRKAPSGKEIPPLDPA |
| NO: | MLAKIQRQMNGAVDVDPKTGEVTPAQPRLFMWEHELQKIMARLKQAPRTHWIDDLP |
| 97 | SHAAQSVVKDLIKALQAMLRERKKRASGIGGRDTGFPKFKKNRYAAGSVYFANTQLR |
| | FEAKRGKAGDPDAVRGEFARVKLPNGVGWMECRMPRHINAAHAYAQATLMGGRIW |
| | RQGENWYLSCQWKMPKPAPLPRAGRTAAIKIAAAIPITTVDNRGQTREYAMPPIDRER |
| | IAAHAAAGRAQSRALEARKRRAKKREAYAKKRHAKKLERGIAAKPPGRARIKLSPGF |
| | YAAAAKLAKLEAEDANAREAWLHEITTQIVRNFDVIAVPRMEVAKLMKKPEPPEEKE |
| | EQVKAPWQGKRRSLKAARVMMRRTAMALIQTTLKYKAVDLRGPQAYEEIAPLDVTA |
| | AACSGCGVLKPEWKMARAKGREIMRCQEPLPGGKTCNTVLTYTRNSARVIGRELAVR |
| | LAERQKA |
| SEQ | MTTQKTYNFCFYDQRFFELSKEAGEVYSRSLEEFWKIYDETGVWLSKFDLQKHMRNK |
| ID | LERKLLHSDSFLGAMQQVHANLASWKQAKKVVPDACPPRKPKFLQAILFKKSQIKYK |
| NO: | NGFLRLTLGTEKEFLYLKWDINIPLPIYGSVTYSKTRGWKINLCLETEVEQKNLSENKY |
| 98 | LSIDLGVKRVATIFDGENTITLSGKKFMGLMHYRNKLNGKTQSRLSHKKKGSNNYKKI |
| | QRAKRKTTDRLLNIQKEMLHKYSSFIVNYAIRNDIGNIIIGDNSSTHDSPNMRGKTNQKI |
| | SQNPEQKLKNYIKYKFESISGRVDIVPEPYTSRKCPHCKNIKKSSPKGRTYKCKKCGFIF |
| | DRDGVGAINIYNENVSFGQIISPGRIRSLTEPIGMKFHNEIYFKSYVAA |
| SEQ | MSVRSFQARVECDKQTMEHLWRTHKVFNERLPEIIKILFKMKRGECGQNDKQKSLYK |
| ID | SISQSILEANAQNADYLLNSVSIKGWKPGTAKKYRNASFTWADDAAKLSSQGIHVYD |
| NO: | KKQVLGDLPGMMSQMVCRQSVEAISGHIELTKKWEKEHNEWLKEKEKWESEDEHKK |
| 99 | YLDLREKFEQFEQSIGGKITKRRGRWHLYLKWLSDNPDFAAWRGNKAVINPLSEKAQI |
| | RINKAKPNKKNSVERDEFFKANPEMKALDNLHGYYERNFVRRRKTKKNPDGFDHKPT |
| | FTLPHPTIHPRWFVFNKPKTNPEGYRKLILPKKAGDLGSLEMRLLTGEKNKGNYPDDW |
| | ISVKFKADPRLSLIRPVKGRRVVRKGKEQGQTKETDSYEFFDKHLKKWRPAKLSGVKL |
| | IFPDKTPKAAYLYFTCDIPDEPLTETAKKIQWLETGDVTKKGKKRKKKVLPHGLVSCA |
| | VDLSMRRGTTGFATLCRYENGKIHILRSRNLWVGYKEGKGCHPYRWTEGPDLGHIAK |
| | HKREIRILRSKRGKPVKGEESHIDLQKHIDYMGEDRFKKAARTIVNFALNTENAASKN |
| | GFYPRADVLLLENLEGLIPDAEKERGINRALAGWNRRHLVERVIEMAKDAGFKRRVF |
| | EIPPYGTSQVCSKCGALGRRYSIIRENNRREIRFGYVEKLFACPNCGYCANADHNASVN |
| | LNRRFLIEDSFKSYYDWKRLSEKKQKEEIETIESKLMDKLCAMHKISRGSISK |
| SEQ | MHLWRTHCVFNQRLPALLKRLFAMRRGEVGGNEAQRQVYQRVAQFVLARDAKDSV |
| ID | DLLNAVSLRKRSANSAFKKKATISCNGQAREVTGEEVFAEAVALASKGVFAYDKDD |
| NO: | MRAGLPDSLFQPLTRDAVACMRSHEELVATWKKEYREWRDRKSEWEAEPEHALYLN |
| 100 | LRPKFEEGEAARGGRFRKRAERDHAYLDWLEANPQLAAWRRKAPPAVVPIDEAGKR |
| | RIARAKAWKQASVRAEEFWKRNPELHALHKIHVQYLREFVRPRRTRRNKRREGFKQR |
| | PTFTMPDPVRHPRWCLFNAPQTSPQGYRLLRLPQSRRTVGSVELRLLTGPSDGAGFPD |
| | AWVNVRFKADPRLAQLRPVKVPRTVTRGKNKGAKVEADGFRYYDDQLLIERDAQVS |
| | GVKLLFRDIRMAPFADKPIEDRLLSATPYLVFAVEIKDEARTERAKAIRFDETSELTKSG |
| | KKRKTLPAGLVSVAVDLDTRGVGFLTRAVIGVPEIQQTHHGVRLLQSRYVAVGQVEA |
| | RASGEAEWSPGPDLAHIARHKREIRRLRQLRGKPVKGERSHVRLQAHIDRMGEDRFK |
| | KAARKIVNEALRGSNPAAGDPYTRADVLLYESLETLLPDAERERGINRALLRWNRAK |
| | LIEHLKRMCDDAGIRHFPVSPFGTSQVCSKCGALGRRYSLARENGRAVIRFGWVERLF |
| | ACPNPECPGRRPDRPDRPFTCNSDHNASVNLHRVFALGDQAVAAFRALAPRDSPARTL |
| | AVKRVEDTLRPQLMRVHKLADAGVDSPF |
| SEQ | MATLVYRYGVRAHGSARQQDAVVSDPAMLEQLRLGHELRNALVGVQHRYEDGKRA |
| ID | VWSGFASVAAADHRVTTGETAVAELEKQARAEHSADRTAATRQGTAESLKAARAAV |
| NO: | KQARADRKAAMAAVAEQAKPKIQALGDDRDAEIKDLYRRFCQDGVLLPRCGRCAGD |
| 101 | LRSDGDCTDCGAAHEPRKLYWATYNAIREDHQTAVKLVEAKRKAGQPARLRFRRWT |
| | GDGTLTVQLQRMHGPACRCVTCAEKLTRRARKTDPQAPAVAADPAYPPTDPPRDPAL |
| | LASGQGKWRNVLQLGTWIPPGEWSAMSRAERRRVGRSHIGWQLGGGRQLTLPVQLH |
| | RQMPADADVAMAQLTRVRVGGRHRMSVALTAKLPDPPQVQGLPPVALHLGWRQRP |
| | DGSLRVATWACPQPLDLPPAVADVVVSHGGRWGEVIMPARWLADAEVPPRLLGRRD |
| | KAMEPVLEALADWLEAHTEACTARMTPALVRRWRSQGRLAGLTNRWRGQPPTGSAE |
| | ILTYLEAWRIQDKLLWERESHLRRRLAARRDDAWRRVASWLARHAGVLVVDDADIA |
| | ELRRRDDPADTDPTMPASAAQAARARAALAAPGRLRHLATITATRDGLGVHTVASAG |
| | LTRLHRKCGHQAQPDPRYAASAVVTCPGCGNGYDQDYNAAMLMLDRQQQP |
| SEQ | MSRVELHRAYKFRLYPTPAQVAELAEWERQLRRLYNLAHSQRLAAMQRHVRPKSPG |
| ID | VLKSECLSCGAVAVAEIGTDGKAKKTVKHAVGCSVLECRSCGGSPDAEGRTAHTAAC |
| NO: | SFVDYYRQGREMTQLLEEDDQLARVVCSARQETLRDLEKAWQRWHKMPGFGKPHF |
| 102 | KKRIDSCRIYFSTPKSWAVDLGYLSFTGVASSVGRIKIRQDRVWPGDAKFSSCHVVRD |
| | VDEWYAVFPLTFTKEIEKPKGGAVGINRGAVHAIADSTGRVVDSPKFYARSLGVIRHR |
| | ARLLDRKVPFGRAVKPSPTKYHGLPKADIDAAAARVNASPGRLVYEARARGSIAAAE |
| | AHLAALVLPAPRQTSQLPSEGRNRERARRFLALAHQRVRRQREWFLHNESAHYAQSY |
| | TKIAIEDWSTKEMTSSEPRDAEEMKRVTRARNRSILDVGWYELGRQIAYKSEATGAEF |
| | AKVDPGLRETETHVPEAIVRERDVDVSGMLRGEAGISGTCSRCGGLLRASASGHADAE |
| | CEVCLHVEVGDVNAAVNVLKRAMFPGAAPPSKEKAKVTIGIKGRKKKRAA |
| SEQ | MSRVELHRAYKFRLYPTPVQVAELSEWERQLRRLYNLGHEQRLLTLTRHLRPKSPGV |
| ID | LKGECLSCDSTQVQEVGADGRPKTTVRHAEQCPTLACRSCGALRDAEGRTAHTVACA |
| NO: | FVDYYRQGREMTELLAADDQLARVVCSARQEVLRDLDKAWQRWRKMPGFGKPRFK |
| 103 | RRTDSCRIYFSTPKAWKLEGGHLSFTGAATTVGAIKMRQDRNWPASVQFSSCHVVRD |
| | VDEWYAVFPLTFVAEVARPKGGAVGINRGAVHAIADSTGRVVDSPRYYARALGVIRH |
| | RARLFDRKVPSGHAVKPSPTKYRGLSAIEVDRVARATGFTPGRVVTEALNRGGVAYA |
| | ECALAAIAVLGHGPERPLTSDGRNREKARKFLALAHQRVRRQREWFLHNESAHYART |
| | YSKIAIEDWSTKEMTASEPQGEETRRVTRSRNRSILDVGWYELGRQLAYKTEATGAEF |
| | AQVDPGLKETETNVPKAIADARDVDVSGMLRGEAGISGTCSKCGGLLRAPASGHADA |
| | ECEICLNVEVGDVNAAVNVLKRAMFPGDAPPASGEKPKVSIGIKGRQKKKKAA |
| SEQ | MEAIATGMSPERRVELGILPGSVELKRAYKFRLYPMKVQQAELSEWERQLRRLYNLA |
| ID | HEQRLAALLRYRDWDFQKGACPSCRVAVPGVHTAACDHVDYFRQAREMTQLLEVD |
| NO: | AQLSRVICCARQEVLRDLDKAWQRWRKKLGGRPRFKRRTDSCRIYLSTPKHWEIAGR |
| 104 | YLRLSGLASSVGEIRIEQDRAFPEGALLSSCSIVRDVDEWYACLPLTFTQPIERAPHRSV |
| | GLNRGVVHALADSDGRVVDSPKFFERALATVQKRSRDLARKVSGSRNAHKARIKLAK |
| | AHQRVRRQRAAFLHQESAYYSKGFDLVALEDMSVRKMTATAGEAPEMGRGAQRDL |
| | NRGILDVGWYELARQIDYKRLAHGGELLRVDPGQTTPLACVTEEQPARGISSACAVCG |
| | IPLARPASGNARMRCTACGSSQVGDVNAAENVLTRALSSAPSGPKSPKASIKIKGRQK |
| | RLGTPANRAGEASGGDPPVRGPVEGGTLAYVVEPVSESQSDT |
| SEQ | MTVRTYKYRAYPTPEQAEALTSWLRFASQLYNAALEHRKNAWGRHDAHGRGFRFW |
| ID | DGDAAPRKKSDPPGRWVYRGGGGAHISKNDQGKLLTEFRREHAELLPPGMPALVQH |
| NO: | EVLARLERSMAAFFQRATKGQKAGYPRWRSEHRYDSLTFGLTSPSKERFDPETGESLG |
| 105 | RGKTVGAGTYHNGDLRLTGLGELRILEHRRIPMGAIPKSVIVRRSGKRWFVSIAMEMP |
| | SVEPAASGRPAVGLDMGVVTWGTAFTADTSAAAALVADLRRMATDPSDCRRLEELE |
| | REAAQLSEVLAHCRARGLDPARPRRCPKELTKLYRRSLHRLGELDRACARIRRRLQAA |
| | HDIAEPVPDEAGSAVLIEGSNAGMRHARRVARTQRRVARRTRAGHAHSNRRKKAVQ |
| | AYARAKERERSARGDHRHKVSRALVRQFEEISVEALDIKQLTVAPEHNPDPQPDLPAH |
| | VQRRRNRGELDAAWGAFFAALDYKAADAGGRVARKPAPHTTQECARCGTLVPKPIS |
| | LRVHRCPACGYTAPRTVNSARNVLQRPLEEPGRAGPSGANGRGVPHAVA |
| SEQ | MNCRYRYRIYPTPGQRQSLARLFGCVRVVWNDALFLCRQSEKLPKNSELQKLCITQA |
| ID | KKTEARGWLGQVSAIPLQQSVADLGVAFKNFFQSRSGKRKGKKVNPPRVKRRNNRQ |
| NO: | GARFTRGGFKVKTSKVYLARIGDIKIKWSRPLPSEPSSVTVIKDCAGQYFLSFVVEVKP |
| 106 | EIKPPKNPSIGIDLGLKTFASCSNGEKIDSPDYSRLYRKLKRCQRRLAKRQRGSKRRER |
| | MRVKVAKLNAQIRDKRKDFLHKLSTKVVNENQVIALEDLNVGGMLKNRKLSRAISQ |
| | AGWYEFRSLCEGKAEKHNRDFRVISRWEPTSQVCSECGYRWGKIDLSVRSIVCINCGV |
| | EHDRDDNASVNIEQAGLKVGVGHTHDSKRTGSACKTSNGAVCVEPSTHREYVQLTLF |
| | DW |
| SEQ | MKSRWTFRCYPTPEQEQHLARTFGCVRFVWNWALRARTDAFRAGERIGYPATDKAL |
| ID | TLLKQQPETVWLNEVSSVCLQQALRDLQVAFSNFFDKRAAHPSFKRKEARQSANYTE |
| NO: | RGFSFDHERRILKLAKIGAIKVKWSRKAIPHPSSIRLIRTASGKYFVSLVVETQPAPMPE |
| 107 | TGESVGVDFGVARLATLSNGERISNPKHGAKWQRRLAFYQKRLARATKGSKRRMRIK |
| | RHVARIHEKIGNSRSDTLHKLSTDLVTRFDLICVEDLNLRGMVKNHSLARSLHDASIGS |
| | AIRMIEEKAERYGKNVVKIDRWFPSSKTCSDCGHIVEQLPLNVREWTCPECGTTHDRD |
| | ANAAANILAVGQTVSAHGGTVRRSRAKASERKSQRSANRQGVNRA |
| SEQ | KEPLNIGKTAKAVFKEIDPTSLNRAANYDASIELNCKECKFKPFKNVKRYEFNFYNNW |
| ID | YRCNPNSCLQSTYKAQVRKVEIGYEKLKNEILTQMQYYPWFGRLYQNFFHDERDKMT |
| NO: | SLDEIQVIGVQNKVFFNTVEKAWREIIKKRFKDNKETMETIPELKHAAGHGKRKLSNK |
| 108 | SLLRRRFAFVQKSFKFVDNSDVSYRSFSNNIACVLPSRIGVDLGGVISRNPKREYIPQEIS |
| | FNAFWKQHEGLKKGRNIEIQSVQYKGETVKRIEADTGEDKAWGKNRQRRFTSLILKL |
| | VPKQGGKKVWKYPEKRNEGNYEYFPIPIEFILDSGETSIRFGGDEGEAGKQKHLVIPFN |
| | DSKATPLASQQTLLENSRFNAEVKSCIGLAIYANYFYGYARNYVISSIYHKNSKNGQAI |
| | TAIYLESIAHNYVKAIERQLQNLLLNLRDFSFMESHKKELKKYFGGDLEGTGGAQKRR |
| | EKEEKIEKEIEQSYLPRLIRLSLTKMVTKQVEM |
| SEQ | ELIVNENKDPLNIGKTAKAVFKEIDPTSINRAANYDASIELACKECKFKPFNNTKRHDF |
| ID | SFYSNWHRCSPNSCLQSTYRAKIRKTEIGYEKLKNEILNQMQYYPWFGRLYQNFFNDQ |
| NO: | RDKMTSLDEIQVTGVQNKIFFNTVEKAWREIIKKRFRDNKETMRTIPDLKNKSGHGSR |
| 109 | KLSNKSLLRRRFAFAQKSFKLVDNSDVSYRAFSNNVACVLPSKIGVDIGGIINKDLKRE |
| | YIPQEITFNVFWKQHDGLKKGRNIEIHSVQYKGEIVKRIEADTGEDKAWGKNRQRRFT |
| | SLILKITPKQGGKKIWKFPEKKNASDYEYFPIPIEFILDNGDASIKFGGEEGEVGKQKHL |
| | LIPFNDSKATPLSSKQMLLETSRFNAEVKSTIGLALYANYFVSYARNYVIKSTYHKNSK |
| | KGQIVTEIYLESISQNFVRAIQRQLQSLMLNLKDWGFMQTHKKELKKYFGSDLEGSKG |
| | GQKRREKEEKIEKEIEASYLPRLIRLSLTKSVTKAEEM |
| SEQ | PEEKTSKLKPNSINLAANYDANEKFNCKECKFHPFKNKKRYEFNFYNNLHGCKSCTKS |
| ID | TNNPAVKRIEIGYQKLKFEIKNQMEAYPWFGRLRINFYSDEKRKMSELNEMQVTGVK |
| NO: | NKIFFDAIECAWREILKKRFRESKETLITIPKLKNKAGHGARKHRNKKLLIRRRAFMKK |
| 110 | NFHFLDNDSISYRSFANNIACVLPSKVGVDIGGIISPDVGKDIKPVDISLNLMWASKEGI |
| | KSGRKVEIYSTQYDGNMVKKIEAETGEDKSWGKNRKRRQTSLLLSIPKPSKQVQEFDF |
| | KEWPRYKDIEKKVQWRGFPIKIIFDSNHNSIEFGTYQGGKQKVLPIPFNDSKTTPLGSK |
| | MNKLEKLRFNSKIKSRLGSAIAANKFLEAARTYCVDSLYHEVSSANAIGKGKIFIEYYL |
| | EILSQNYIEAAQKQLQRFIESIEQWFVADPFQGRLKQYFKDDLKRAKCFLCANREVQT |
| | TCYAAVKLHKSCAEKVKDKNKELAIKERNNKEDAVIKEVEASNYPRVIRLKLTKTITN |
| | KAM |
| SEQ | SESENKIIEQYYAFLYSFRDKYEKPEFKNRGDIKRKLQNKWEDFLKEQNLKNDKKLSN |
| ID | YIFSNRNFRRSYDREEENEEGIDEKKSKPKRINCFEKEKNLKDQYDKDAINASANKDG |
| NO: | AQKWGCFECIFFPMYKIESGDPNKRIIINKTRFKLFDFYLNLKGCKSCLRSTYHPYRSN |
| 111 | VYIESNYDKLKREIGNFLQQKNIFQRMRKAKVSEGKYLTNLDEYRLSCVAMHFKNRW |
| | LFFDSIQKVLRETIKQRLKQMRESYDEQAKTKRSKGHGRAKYEDQVRMIRRRAYSAQ |
| | AHKLLDNGYITLFDYDDKEINKVCLTAINQEGFDIGGYLNSDIDNVMPPIEISFHLKWK |
| | YNEPILNIESPFSKAKISDYLRKIREDLNLERGKEGKARSKKNVRRKVLASKGEDGYKK |
| | IFTDFFSKWKEELEGNAMERVLSQSSGDIQWSKKKRIHYTTLVLNINLLDKKGVGNLK |
| | YYEIAEKTKILSFDKNENKFWPITIQVLLDGYEIGTEYDEIKQLNEKTSKQFTIYDPNTKI |
| | IKIPFTDSKAVPLGMLGINIATLKTVKKTERDIKVSKIFKGGLNSKIVSKIGKGIYAGYFP |
| | TVDKEILEEVEEDTLDNEFSSKSQRNIFLKSIIKNYDKMLKEQLFDFYSFLVRNDLGVRF |
| | LTDRELQNIEDESFNLEKRFFETDRDRIARWFDNTNTDDGKEKFKKLANEIVDSYKPRL |
| | IRLPVVRVIKRIQPVKQREM |
| SEQ | KYSTRDFSELNEIQVTACKQDEFFKVIQNAWREIIKKRFLENRENFIEKKIFKNKKGRG |
| ID | KRQESDKTIQRNRASVMKNFQLIENEKIILRAPSGHVACVFPVKVGLDIGGFKTDDLEK |
| NO: | NIFPPRTITINVFWKNRDRQRKGRKLEVWGIKARTKLIEKVHKWDKLEEVKKKRLKSL |
| 112 | EQKQEKSLDNWSEVNNDSFYKVQIDELQEKIDKSLKGRTMNKILDNKAKESKEAEGL |
| | YIEWEKDFEGEMLRRIEASTGGEEKWGKRRQRRHTSLLLDIKNNSRGSKEIINFYSYAK |
| | QGKKEKKIEFFPFPLTITLDAEEESPLNIKSIPIEDKNATSKYFSIPFTETRATPLSILGDRV |
| | QKFKTKNISGAIKRNLGSSISSCKIVQNAETSAKSILSLPNVKEDNNMEIFINTMSKNYF |
| | RAMMKQMESFIFEMEPKTLIDPYKEKAIKWFEVAASSRAKRKLKKLSKADIKKSELLL |
| | SNTEEFEKEKQEKLEALEKEIEEFYLPRIVRLQLTKTILETPVM |
| SEQ | KKLQLLGHKILLKEYDPNAVNAAANFETSTAELCGQCKMKPFKNKRRFQYTFGKNYH |
| ID | GCLSCIQNVYYAKKRIVQIAKEELKHQLTDSIASIPYKYTSLFSNTNSIDELYILKQERA |
| NO: | AFFSNTNSIDELYITGIENNIAFKVISAIWDEIIKKRRQRYAESLTDTGTVKANRGHGGT |
| 113 | AYKSNTRQEKIRALQKQTLHMVTNPYISLARYKNNYIVATLPRTIGMHIGAIKDRDPQ |
| | KKLSDYAINFNVFWSDDRQLIELSTVQYTGDMVRKIEAETGENNKWGENMKRTKTSL |
| | LLEILTKKTTDELTFKDWAFSTKKEIDSVTKKTYQGFPIGIIFEGNESSVKFGSQNYFPLP |
| | FDAKITPPTAEGFRLDWLRKGSFSSQMKTSYGLAIYSNKVTNAIPAYVIKNMFYKIARA |
| | ENGKQIKAKFLKKYLDIAGNNYVPFIIMQHYRVLDTFEEMPISQPKVIRLSLTKTQHIIIK |
| | KDKTDSKM |
| SEQ | NTSNLINLGKKAINISANYDANLEVGCKNCKFLSSNGNFPRQTNVKEGCHSCEKSTYE |
| ID | PSIYLVKIGERKAKYDVLDSLKKFTFQSLKYQSKKSMKSRNKKPKELKEFVIFANKNK |
| NO: | AFDVIQKSYNHLILQIKKEINRMNSKKRKKNHKRRLFRDREKQLNKLRLIESSNLFLPR |
| 114 | ENKGNNHVFTYVAIHSVGRDIGVIGSYDEKLNFETELTYQLYFNDDKRLLYAYKPKQ |
| | NKIIKIKEKLWNLRKEKEPLDLEYEKPLNKSITFSIKNDNLFKVSKDLMLRRAKFNIQG |
| | KEKLSKEERKINRDLIKIKGLVNSMSYGRFDELKKEKNIWSPHIYREVRQKEIKPCLIKN |
| | GDRIEIFEQLKKKMERLRRFREKRQKKISKDLIFAERIAYNFHTKSIKNTSNKINIDQEA |
| | KRGKASYMRKRIGYETFKNKYCEQCLSKGNVYRNVQKGCSCFENPFDWIKKGDENL |
| | LPKKNEDLRVKGAFRDEALEKQIVKIAFNIAKGYEDFYDNLGESTEKDLKLKFKVGTT |
| | INEQESLKL |
| SEQ | TSNPIKLGKKAINISANYDSNLQIGCKNCKFLSYNGNFPRQTNVKEGCHSCEKSTYEPP |
| ID | VYTVRIGERRSKYDVLDSLKKFIFLSLKYRQSKKMKTRSKGIRGLEEFVISANLKKAM |
| NO: | DVIQKSYRHLILNIKNEIVRMNGKKRNKNHKRLLFRDREKQLNKLRLIEGSSFFKPPTV |
| 115 | KGDNSIFTCVAIHNIGRDIGIAGDYFDKLEPKIELTYQLYYEYNPKKESEINKRLLYAYK |
| | PKQNKIIEIKEKLWNLRKEKSPLDLEYEKPLTKSITFLVKRDGVFRISKDLMLRKAKFII |
| | QGKEKLSKEERKINRDLIKIKSNIISLTYGRFDELKKDKTIWSPHIFRDVKQGKITPCIER |
| | KGDRMDIFQQLRKKSERLRENRKKRQKKISKDLIFAERIAYNFHTKSIKNTSNLINIKHE |
| | AKRGKASYMRKRIGNETFRIKYCEQCFPKNNVYKNVQKGCSCFEDPFEYIKKGNEDLI |
| | PNKNQDLKAKGAFRDDALEKQIIKVAFNIAKGYEDFYENLKKTTEKDIRLKFKVGTIIS |
| | EEM |
| SEQ | NNSINLSKKAINISANYDANLQVRCKNCKFLSSNGNFPRQTDVKEGCHSCEKSTYEPPV |
| ID | YDVKIGEIKAKYEVLDSLKKFTFQSLKYQLSKSMKFRSKKIKELKEFVIFAKESKALNV |
| NO: | INRSYKHLILNIKNDINRMNSKKRIKNHKGRLFLDRQKQLSKLKLIEGSSFFVPAKNVG |
| 116 | NKSVFTCVAIHSIGRDIGIAGLYDSFTKPVNEITYQIFFSGERRLLYAYKPKQLKILSIKE |
| | NLWSLKNEKKPLDLLYEKPLGKNLNFNVKGGDLFRVSKDLMIRNAKFNVHGRQRLS |
| | DEERLINRNFIKIKGEVVSLSYGRFEELKKDRKLWSPHIFKDVRQNKIKPCLVMQGQRI |
| | DIFEQLKRKLELLKKIRKSRQKKLSKDLIFGERIAYNFHTKSIKNTSNKINIDSDAKRGR |
| | ASYMRKRIGNETFKLKYCDVCFPKANVYRRVQNGCSCSENPYNYIKKGDKDLLPKKD |
| | EGLAIKGAFRDEKLNKQIIKVAFNIAKGYEDFYDDLKKRTEKDVDLKFKIGTTVLDQK |
| | PMEIFDGIVITWL |
| SEQ | LLTTVVETNNLAKKAINVAANFDANIDRQYYRCTPNLCRFIAQSPRETKEKDAGCSSC |
| ID | TQSTYDPKVYVIKIGKLLAKYEILKSLKRFLFMNRYFKQKKTERAQQKQKIGTELNEM |
| NO: | SIFAKATNAMEVIKRATKHCTYDIIPETKSLQMLKRRRHRVKVRSLLKILKERRMKIKK |
| 117 | IPNTFIEIPKQAKKNKSDYYVAAALKSCGIDVGLCGAYEKNAEVEAEYTYQLYYEYKG |
| | NSSTKRILYCYNNPQKNIREFWEAFYIQGSKSHVNTPGTIRLKMEKFLSPITIESEALDFR |
| | VWNSDLKIRNGQYGFIKKRSLGKEAREIKKGMGDIKRKIGNLTYGKSPSELKSIHVYRT |
| | ERENPKKPRAARKKEDNFMEIFEMQRKKDYEVNKKRRKEATDAAKIMDFAEEPIRHY |
| | HTNNLKAVRRIDMNEQVERKKTSVFLKRIMQNGYRGNYCRKCIKAPEGSNRDENVLE |
| | KNEGCLDCIGSEFIWKKSSKEKKGLWHTNRLLRRIRLQCFTTAKAYENFYNDLFEKKE |
| | SSLDIIKLKVSITTKSM |
| SEQ | ASTMNLAKQAINFAANYDSNLEIGCKGCKFMSTWSKKSNPKFYPRQNNQANKCHSCT |
| ID | YSTGEPEVPIIEIGERAAKYKIFTALKKFVFMSVAYKERRRQRFKSKKPKELKELAICSN |
| NO: | REKAMEVIQKSVVHCYGDVKQEIPRIRKIKVLKNHKGRLFYKQKRSKIKIAKLEKGSFF |
| 118 | KTFIPKVHNNGCHSCHEASLNKPILVTTALNTIGADIGLINDYSTIAPTETDISWQVYYE |
| | FIPNGDSEAVKKRLLYFYKPKGALIKSIRDKYFKKGHENAVNTGFFKYQGKIVKGPIKF |
| | VNNELDFARKPDLKSMKIKRAGFAIPSAKRLSKEDREINRESIKIKNKIYSLSYGRKKTL |
| | SDKDIIKHLYRPVRQKGVKPLEYRKAPDGFLEFFYSLKRKERRLRKQKEKRQKDMSEII |
| | DAADEFAWHRHTGSIKKTTNHINFKSEVKRGKVPIMKKRIANDSFNTRHCGKCVKQG |
| | NAINKYYIEKQKNCFDCNSIEFKWEKAALEKKGAFKLNKRLQYIVKACFNVAKAYES |
| | FYEDFRKGEEESLDLKFKIGTTTTLKQYPQNKARAM |
| SEQ | HSHNLMLTKLGKQAINFAANYDANLEIGCKNCKFLSYSPKQANPKKYPRQTDVHEDG |
| ID | NIACHSCMQSTKEPPVYIVPIGERKSKYEILTSLNKFTFLALKYKEKKRQAFRAKKPKE |
| NO: | LQELAIAFNKEKAIKVIDKSIQHLILNIKPEIARIQRQKRLKNRKGKLLYLHKRYAIKMG |
| 119 | LIKNGKYFKVGSPKKDGKKLLVLCALNTIGRDIGIIGNIEENNRSETEITYQLYFDCLDA |
| | NPNELRIKEIEYNRLKSYERKIKRLVYAYKPKQTKILEIRSKFFSKGHENKVNTGSFNFE |
| | NPLNKSISIKVKNSAFDFKIGAPFIMLRNGKFHIPTKKRLSKEEREINRTLSKIKGRVFRL |
| | TYGRNISEQGSKSLHIYRKERQHPKLSLEIRKQPDSFIDEFEKLRLKQNFISKLKKQRQK |
| | KLADLLQFADRIAYNYHTSSLEKTSNFINYKPEVKRGRTSYIKKRIGNEGFEKLYCETCI |
| | KSNDKENAYAVEKEELCFVCKAKPFTWKKTNKDKLGIFKYPSRIKDFIRAAFTVAKSY |
| | NDFYENLKKKDLKNEIFLKFKIGLILSHEKKNHISIAKSVAEDERISGKSIKNILNKSIKL |
| | EKNCYSCFFHKEDM |
| SEQ | SLERVIDKRNLAKKAINIAANFDANINKGFYRCETNQCMFIAQKPRKTNNTGCSSCLQS |
| ID | TYDPVIYVVKVGEMLAKYEILKSLKRFVFMNRSFKQKKTEKAKQKERIGGELNEMSIF |
| NO: | ANAALAMGVIKRAIRHCHVDIRPEINRLSELKKTKHRVAAKSLVKIVKQRKTKWKGIP |
| 120 | NSFIQIPQKARNKDADFYVASALKSGGIDIGLCGTYDKKPHADPRWTYQLYFDTEDES |
| | EKRLLYCYNDPQAKIRDFWKTFYERGNPSMVNSPGTIEFRMEGFFEKMTPISIESKDFD |
| | FRVWNKDLLIRRGLYEIKKRKNLNRKAREIKKAMGSVKRVLANMTYGKSPTDKKSIP |
| | VYRVEREKPKKPRAVRKEENELADKLENYRREDFLIRNRRKREATEIAKIIDAAEPPIR |
| | HYHTNHLRAVKRIDLSKPVARKNTSVFLKRIMQNGYRGNYCKKCIKGNIDPNKDECR |
| | LEDIKKCICCEGTQNIWAKKEKLYTGRINVLNKRIKQMKLECFNVAKAYENFYDNLA |
| | ALKEGDLKVLKLKVSIPALNPEASDPEEDM |
| SEQ | NASINLGKRAINLSANYDSNLVIGCKNCKFLSFNGNFPRQTNVREGCHSCDKSTYAPE |
| ID | VYIVKIGERKAKYDVLDSLKKFTFQSLKYQIKKSMRERSKKPKELLEFVIFANKDKAF |
| NO: | NVIQKSYEHLILNIKQEINRMNGKKRIKNHKKRLFKDREKQLNKLRLIGSSSLFFPREN |
| 121 | KGDKDLFTYVAIHSVGRDIGVAGSYESHIEPISDLTYQLFINNEKRLLYAYKPKQNKIIE |
| | LKENLWNLKKEKKPLDLEFTKPLEKSITFSVKNDKLFKVSKDLMLRQAKFNIQGKEKL |
| | SKEERQINRDFSKIKSNVISLSYGRFEELKKEKNIWSPHIYREVKQKEIKPCIVRKGDRIE |
| | LFEQLKRKMDKLKKFRKERQKKISKDLNFAERIAYNFHTKSIKNTSNKINIDQEAKRG |
| | KASYMRKRIGNESFRKKYCEQCFSVGNVYHNVQNGCSCFDNPIELIKKGDEGLIPKGK |
| | EDRKYKGALRDDNLQMQIIRVAFNIAKGYEDFYNNLKEKTEKDLKLKFKIGTTISTQE |
| | SNNKEM |
| SEQ | SNLIKLGKQAINFAANYDANLEVGCKNCKFLSSTNKYPRQTNVHLDNKMACRSCNQS |
| ID | TMEPAIYIVRIGEKKAKYDIYNSLTKFNFQSLKYKAKRSQRFKPKQPKELQELSIAVRK |
| NO: | EKALDIIQKSIDHLIQDIRPEIPRIKQQKRYKNHVGKLFYLQKRRKNKLNLIGKGSFFKV |
| 122 | FSPKEKKNELLVICALTNIGRDIGLIGNYNTIINPLFEVTYQLYYDYIPKKNNKNVQRRL |
| | LYAYKSKNEKILKLKEAFFKRGHENAVNLGSFSYEKPLEKSLTLKIKNDKDDFQVSPSL |
| | RIRTGRFFVPSKRNLSRQEREINRRLVKIKSKIKNMTYGKFETARDKQSVHIFRLERQKE |
| | KLPLQFRKDEKEFMEEFQKLKRRTNSLKKLRKSRQKKLADLLQLSEKVVYNNHTGTL |
| | KKTSNFLNFSSSVKRGKTAYIKELLGQEGFETLYCSNCINKGQKTRYNIETKEKCFSCK |
| | DVPFVWKKKSTDKDRKGAFLFPAKLKDVIKATFTVAKAYEDFYDNLKSIDEKKPYIKF |
| | KIGLILAHVRHEHKARAKEEAGQKNIYNKPIKIDKNCKECFFFKEEAM |
| SEQ | NTTRKKFRKRTGFPQSDNIKLAYCSAIVRAANLDADIQKKHNQCNPNLCVGIKSNEQS |
| ID | RKYEHSDRQALLCYACNQSTGAPKVDYIQIGEIGAKYKILQMVNAYDFLSLAYNLTKL |
| NO: | RNGKSRGHQRMSQLDEVVIVADYEKATEVIKRSINHLLDDIRGQLSKLKKRTQNEHIT |
| 123 | EHKQSKIRRKLRKLSRLLKRRRWKWGTIPNPYLKNWVFTKKDPELVTVALLHKLGRD |
| | IGLVNRSKRRSKQKLLPKVGFQLYYKWESPSLNNIKKSKAKKLPKRLLIPYKNVKLFD |
| | NKQKLENAIKSLLESYQKTIKVEFDQFFQNRTEEIIAEEQQTLERGLLKQLEKKKNEFA |
| | SQKKALKEEKKKIKEPRKAKLLMEESRSLGFLMANVSYALFNTTIEDLYKKSNVVSGC |
| | IPQEPVVVFPADIQNKGSLAKILFAPKDGFRIKFSGQHLTIRTAKFKIRGKEIKILTKTKR |
| | EILKNIEKLRRVWYREQHYKLKLFGKEVSAKPRFLDKRKTSIERRDPNKLADQTDDRQ |
| | AELRNKEYELRHKQHKMAERLDNIDTNAQNLQTLSFWVGEADKPPKLDEKDARGFG |
| | VRTCISAWKWFMEDLLKKQEEDPLLKLKLSIM |
| SEQ | PKKPKFQKRTGFPQPDNLRKEYCLAIVRAANLDADFEKKCTKCEGIKTNKKGNIVKGR |
| ID | TYNSADKDNLLCYACNISTGAPAVDYVFVGALEAKYKILQMVKAYDFHSLAYNLAK |
| NO: | LWKGRGRGHQRMGGLNEVVIVSNNEKALDVIEKSLNHFHDEIRGELSRLKAKFQNEH |
| 124 | LHVHKESKLRRKLRKISRLLKRRRWKWDVIPNSYLRNFTFTKTRPDFISVALLHRVGR |
| | DIGLVTKTKIPKPTDLLPQFGFQIYYTWDEPKLNKLKKSRLRSEPKRLLVPYKKIELYK |
| | NKSVLEEAIRHLAEVYTEDLTICFKDFFETQKRKFVSKEKESLKRELLKELTKLKKDFS |
| | ERKTALKRDRKEIKEPKKAKLLMEESRSLGFLAANTSYALFNLIAADLYTKSKKACST |
| | KLPRQLSTILPLEIKEHKSTTSLAIKPEEGFKIRFSNTHLSIRTPKFKMKGADIKALTKRK |
| | REILKNATKLEKSWYGLKHYKLKLYGKEVAAKPRFLDKRNPSIDRRDPKELMEQIENR |
| | RNEVKDLEYEIRKGQHQMAKRLDNVDTNAQNLQTKSFWVGEADKPPELDSMEAKKL |
| | GLRTCISAWKWFMKDLVLLQEKSPNLKLKLSLTEM |
| SEQ | KFSKRQEGFLIPDNIDLYKCLAIVRSANLDADVQGHKSCYGVKKNGTYRVKQNGKKG |
| ID | VKEKGRKYVFDLIAFKGNIEKIPHEAIEEKDQGRVIVLGKFNYKLILNIEKNHNDRASL |
| NO: | EIKNKIKKLVQISSLETGEFLSDLLSGKIGIDEVYGIIEPDVFSGKELVCKACQQSTYAPL |
| 125 | VEYMPVGELDAKYKILSAIKGYDFLSLAYNLSRNRANKKRGHQKLGGGELSEVVISA |
| | NYDKALNVIKRSINHYHVEIKPEISKLKKKMQNEPLKVMKQARIRRELHQLSRKVKRL |
| | KWKWGMIPNPELQNIIFEKKEKDFVSYALLHTLGRDIGLFKDTSMLQVPNISDYGFQIY |
| | YSWEDPKLNSIKKIKDLPKRLLIPYKRLDFYIDTILVAKVIKNLIELYRKSYVYETFGEE |
| | YGYAKKAEDILFDWDSINLSEGIEQKIQKIKDEFSDLLYEARESKRQNFVESFENILGLY |
| | DKNFASDRNSYQEKIQSMIIKKQQENIEQKLKREFKEVIERGFEGMDQNKKYYKVLSP |
| | NIKGGLLYTDTNNLGFFRSHLAFMLLSKISDDLYRKNNLVSKGGNKGILDQTPETMLT |
| | LEFGKSNLPNISIKRKFFNIKYNSSWIGIRKPKFSIKGAVIREITKKVRDEQRLIKSLEGV |
| | WHKSTHFKRWGKPRFNLPRHPDREKNNDDNLMESITSRREQIQLLLREKQKQQEKMA |
| | GRLDKIDKEIQNLQTANFQIKQIDKKPALTEKSEGKQSVRNALSAWKWFMEDLIKYQK |
| | RTPILQLKLAKM |
| SEQ | KFSKRQEGFVIPENIGLYKCLAIVRSANLDADVQGHVSCYGVKKNGTYVLKQNGKKSI |
| ID | REKGRKYASDLVAFKGDIEKIPFEVIEEKKKEQSIVLGKFNYKLVLDVMKGEKDRASL |
| NO: | TMKNKSKKLVQVSSLGTDEFLLTLLNEKFGIEEIYGIIEPEVFSGKKLVCKACQQSTYA |
| 126 | PLVEYMPVGELDSKYKILSAIKGYDFLSLAYNLARHRSNKKRGHQKLGGGELSEVVIS |
| | ANNAKALNVIKRSLNHYYSEIKPEISKLRKKMQNEPLKVGKQARMRRELHQLSRKVK |
| | RLKWKWGKIPNLELQNITFKESDRDFISYALLHTLGRDIGMFNKTEIKMPSNILGYGFQ |
| | IYYDWEEPKLNTIKKSKNTPKRILIPYKKLDFYNDSILVARAIKELVGLFQESYEWEIFG |
| | NEYNYAKEAEVELIKLDEESINGNVEKKLQRIKENFSNLLEKAREKKRQNFIESFESIAR |
| | LYDESFTADRNEYQREIQSFIIEKQKQSIEKKLKNEFKKIVEKKFNEQEQGKKHYRVLN |
| | PTIINEFLPKDKNNLGFLRSKIAFILLSKISDDLYKKSNAVSKGGEKGIIKQQPETILDLEF |
| | SKSKLPSINIKKKLFNIKYTSSWLGIRKPKFNIKGAKIREITRRVRDVQRTLKSAESSWY |
| | ASTHFRRWGFPRFNQPRHPDKEKKSDDRLIESITLLREQIQILLREKQKGQKEMAGRLD |
| | DVDKKIQNLQTANFQIKQTGDKPALTEKSAGKQSFRNALSAWKWFMENLLKYQNKT |
| | PDLKLKIARTVM |
| SEQ | KWIEPNNIDFNKCLAITRSANLDADVQGHKMCYGIKTNGTYKAIGKINKKHNTGIIEK |
| ID | RRTYVYDLIVTKEKNEKIVKKTDFMAIDEEIEFDEKKEKLLKKYIKAEVLGTGELIRKD |
| NO: | LNDGEKFDDLCSIEEPQAFRRSELVCKACNQSTYASDIRYIPIGEIEAKYKILKAIKGYD |
| 127 | FLSLKYNLGRLRDSKKRGHQKMGQGELKEFVICANKEKALDVIKRSLNHYLNEVKDE |
| | ISRLNKKMQNEPLKVNDQARWRRELNQISRRLKRLKWKWGEIPNPELKNLIFKSSRPE |
| | FVSYALIHTLGRDIGLINETELKPNNIQEYGFQIYYKWEDPELNHIKKVKNIPKRFIIPYK |
| | NLDLFGKYTILSRAIEGILKLYSSSFQYKSFKDPNLFAKEGEKKITNEDFELGYDEKIKKI |
| | KDDFKSYKKALLEKKKNTLEDSLNSILSVYEQSLLTEQINNVKKWKEGLLKSKESIHK |
| | QKKIENIEDIISRIEELKNVEGWIRTKERDIVNKEETNLKREIKKELKDSYYEEVRKDFS |
| | DLKKGEESEKKPFREEPKPIVIKDYIKFDVLPGENSALGFFLSHLSFNLFDSIQYELFEKS |
| | RLSSSKHPQIPETILDL |
| SEQ | FRKFVKRSGAPQPDNLNKYKCIAIVRAANLDADIMSNESSNCVMCKGIKMNKRKTAK |
| ID | GAAKTTELGRVYAGQSGNLLCTACTKSTMGPLVDYVPIGRIRAKYTILRAVKEYDFLS |
| NO: | LAYNLARTRVSKKGGRQKMHSLSELVIAAEYEIAWNIIKSSVIHYHQETKEEISGLRKK |
| 128 | LQAEHIHKNKEARIRREMHQISRRIKRLKWKWHMIPNSELHNFLFKQQDPSFVAVALL |
| | HTLGRDIGMINKPKGSAKREFIPEYGFQIYYKWMNPKLNDINKQKYRKMPKRSLIPYK |
| | NLNVFGDRELIENAMHKLLKLYDENLEVKGSKFFKTRVVAISSKESEKLKRDLLWKG |
| | ELAKIKKDFNADKNKMQELFKEVKEPKKANALMKQSRNMGFLLQNISYGALGLLAN |
| | RMYEASAKQSKGDATKQPSIVIPLEMEFGNAFPKLLLRSGKFAMNVSSPWLTIRKPKF |
| | VIKGNKIKNITKLMKDEKAKLKRLETSYHRATHFRPTLRGSIDWDSPYFSSPKQPNTHR |
| | RSPDRLSADITEYRGRLKSVEAELREGQRAMAKKLDSVDMTASNLQTSNFQLEKGED |
| | PRLTEIDEKGRSIRNCISSWKKFMEDLMKAQEANPVIKIKIALKDESSVLSEDSM |
| SEQ | KFHPENLNKSYCLAIVRAANLDADIQGHINCIGIKSNKSDRNYENKLESLQNVELLCKA |
| ID | CTKSTYKPNINSVPVGEKKAKYSILSEIKKYDFNSLVYNLKKYRKGKSRGHQKLNELR |
| NO: | ELVITSEYKKALDVINKSVNHYLVNIKNKMSKLKKILQNEHIHVGTLARIRRERNRISR |
| 129 | KLDHYRKKWKFVPNKILKNYVFKNQSPDFVSVALLHKLGRDIGLITKTAILQKSFPEYS |
| | LQLYYKYDTPKLNYLKKSKFKSLPKRILISYKYPKFDINSNYIEESIDKLLKLYEESPIYK |
| | NNSKIIEFFKKSEDNLIKSENDSLKRGIMKEFEKVTKNFSSKKKKLKEELKLKNEDKNS |
| | KMLAKVSRPIGFLKAYLSYMLFNIISNRIFEFSRKSSGRIPQLPSCIINLGNQFENFKNEL |
| | QDSNIGSKKNYKYFCNLLLKSSGFNISYEEEHLSIKTPNFFINGRKLKEITSEKKKIRKEN |
| | EQLIKQWKKLTFFKPSNLNGKKTSDKIRFKSPNNPDIERKSEDNIVENIAKVKYKLEDL |
| | LSEQRKEFNKLAKKHDGVDVEAQCLQTKSFWIDSNSPIKKSLEKKNEKVSVKKKMKA |
| | IRSCISAWKWFMADLIEAQKETPMIKLKLALM |
| SEQ | TTLVPSHLAGIEVMDETTSRNEDMIQKETSRSNEDENYLGVKNKCGINVHKSGRGSSK |
| ID | HEPNMPPEKSGEGQMPKQDSTEMQQRFDESVTGETQVSAGATASIKTDARANSGPRV |
| NO: | GTARALIVKASNLDRDIKLGCKPCEYIRSELPMGKKNGCNHCEKSSDIASVPKVESGFR |
| 130 | KAKYELVRRFESFAADSISRHLGKEQARTRGKRGKKDKKEQMGKVNLDEIAILKNESL |
| | IEYTENQILDARSNRIKEWLRSLRLRLRTRNKGLKKSKSIRRQLITLRRDYRKWIKPNPY |
| | RPDEDPNENSLRLHTKLGVDIGVQGGDNKRMNSDDYETSFSITWRDTATRKICFTKPK |
| | GLLPRHMKFKLRGYPELILYNEELRIQDSQKFPLVDWERIPIFKLRGVSLGKKKVKALN |
| | RITEAPRLVVAKRIQVNIESKKKKVLTRYVYNDKSINGRLVKAEDSNKDPLLEFKKQA |
| | EEINSDAKYYENQEIAKNYLWGCEGLHKNLLEEQTKNPYLAFKYGFLNIV |
| SEQ | LDFKRTCSQELVLLPEIEGLKLSGTQGVTSLAKKLINKAANVDRDESYGCHHCIHTRTS |
| ID | LSKPVKKDCNSCNQSTNHPAVPITLKGYKIAFYELWHRFTSWAVDSISKALHRNKVM |
| NO: | GKVNLDEYAVVDNSHIVCYAVRKCYEKRQRSVRLHKRAYRCRAKHYNKSQPKVGRI |
| 131 | YKKSKRRNARNLKKEAKRYFQPNEITNGSSDALFYKIGVDLGIAKGTPETEVKVDVSI |
| | CFQVYYGDARRVLRVRKMDELQSFHLDYTGKLKLKGIGNKDTFTIAKRNESLKWGST |
| | KYEVSRAHKKFKPFGKKGSVKRKCNDYFRSIASWSCEAASQRAQSNLKNAFPYQKAL |
| | VKCYKNLDYKGVKKNDMWYRLCSNRIFRYSRIAEDIAQYQSDKGKAKFEFVILAQSV |
| | AEYDISAIM |
| SEQ | VFLTDDKRKTALRKIRSAFRKTAEIALVRAQEADSLDRQAKKLTIETVSFGAPGAKNA |
| ID | FIGSLQGYNWNSHRANVPSSGSAKDVFRITELGLGIPQSAHEASIGKSFELVGNVVRYT |
| NO: | ANLLSKGYKKGAVNKGAKQQREIKGKEQLSFDLISNGPISGDKLINGQKDALAWWLI |
| 132 | DKMGFHIGLAMEPLSSPNTYGITLQAFWKRHTAPRRYSRGVIRQWQLPFGRQLAPLIH |
| | NFFRKKGASIPIVLTNASKKLAGKGVLLEQTALVDPKKWWQVKEQVTGPLSNIWERS |
| | VPLVLYTATFTHKHGAAHKRPLTLKVIRISSGSVFLLPLSKVTPGKLVRAWMPDINILR |
| | DGRPDEAAYKGPDLIRARERSFPLAYTCVTQIADEWQKRALESNRDSITPLEAKLVTGS |
| | DLLQIHSTVQQAVEQGIGGRISSPIQELLAKDALQLVLQQLFMTVDLLRIQWQLKQEV |
| | ADGNTSEKAVGWAIRISNIHKDAYKTAIEPCTSALKQAWNPLSGFEERTFQLDASIVRK |
| | RSTAKTPDDELVIVLRQQAAEMTVAVTQSVSKELMELAVRHSATLHLLVGEVASKQL |
| | SRSADKDRGAMDHWKLLSQSM |
| SEQ | EDLLQKALNTATNVAAIERHSCISCLFTESEIDVKYKTPDKIGQNTAGCQSCTFRVGYS |
| ID | GNSHTLPMGNRIALDKLRETIQRYAWHSLLFNVPPAPTSKRVRAISELRVAAGRERLFT |
| NO: | VITFVQTNILSKLQKRYAANWTPKSQERLSRLREEGQHILSLLESGSWQQKEVVREDQ |
| 133 | DLIVCSALTKPGLSIGAFCRPKYLKPAKHALVLRLIFVEQWPGQIWGQSKRTRRMRRR |
| | KDVERVYDISVQAWALKGKETRISECIDTMRRHQQAYIGVLPFLILSGSTVRGKGDCPI |
| | LKEITRMRYCPNNEGLIPLGIFYRGSANKLLRVVKGSSFTLPMWQNIETLPHPEPFSPEG |
| | WTATGALYEKNLAYWSALNEAVDWYTGQILSSGLQYPNQNEFLARLQNVIDSIPRKW |
| | FRPQGLKNLKPNGQEDIVPNEFVIPQNAIRAHHVIEWYHKTNDLVAKTLLGWGSQTTL |
| | NQTRPQGDLRFTYTRYYFREKEVPEV |
| SEQ | VPKKKLMRELAKKAVFEAIFNDPIPGSFGCKRCTLIDGARVTDAIEKKQGAKRCAGCE |
| ID | PCTFHTLYDSVKHALPAATGCDRTAIDTGLWEILTALRSYNWMSFRRNAVSDASQKQ |
| NO: | VWSIEELAIWADKERALRVILSALTHTIGKLKNGFSRDGVWKGGKQLYENLAQKDLA |
| 134 | KGLFANGEIFGKELVEADHDMLAWTIVPNHQFHIGLIRGNWKPAAVEASTAFDARWL |
| | TNGAPLRDTRTHGHRGRRFNRTEKLTVLCIKRDGGVSEEFRQERDYELSVMLLQPKN |
| | KLKPEPKGELNSFEDLHDHWWFLKGDEATALVGLTSDPTVGDFIQLGLYIRNPIKAHG |
| | ETKRRLLICFEPPIKLPLRRAFPSEAFKTWEPTINVFRNGRRDTEAYYDIDRARVFEFPET |
| | RVSLEHLSKQWEVLRLEPDRENTDPYEAQQNEGAELQVYSLLQEAAQKMAPKVVIDP |
| | FGQFPLELFSTFVAQLFNAPLSDTKAKIGKPLDSGFVVESHLHLLEEDFAYRDFVRVTF |
| | MGTEPTFRVIHYSNGEGYWKKTVLKGKNNIRTALIPEGAKAAVDAYKNKRCPLTLEA |
| | AILNEEKDRRLVLGNKALSLLAQTARGNLTILEALAAEVLRPLSGTEGVVHLHACVTR |
| | HSTLTESTETDNM |
| SEQ | VEKLFSERLKRAMWLKNEAGRAPPAETLTLKHKRVSGGHEKVKEELQRVLRSLSGTN |
| ID | QAAWNLGLSGGREPKSSDALKGEKSRVVLETVVFHSGHNRVLYDVIEREDQVHQRSS |
| NO: | IMHMRRKGSNLLRLWGRSGKVRRKMREEVAEIKPVWHKDSRWLAIVEEGRQSVVGI |
| 135 | SSAGLAVFAVQESQCTTAEPKPLEYVVSIWFRGSKALNPQDRYLEFKKLKTTEALRGQ |
| | QYDPIPFSLKRGAGCSLAIRGEGIKFGSRGPIKQFFGSDRSRPSHADYDGKRRLSLFSKY |
| | AGDLADLTEEQWNRTVSAFAEDEVRRATLANIQDFLSISHEKYAERLKKRIESIEEPVS |
| | ASKLEAYLSAIFETFVQQREALASNFLMRLVESVALLISLEEKSPRVEFRVARYLAESK |
| | EGFNRKAM |
| SEQ | VVITQSELYKERLLRVMEIKNDRGRKEPRESQGLVLRFTQVTGGQEKVKQKLWLIFEG |
| ID | FSGTNQASWNFGQPAGGRKPNSGDALKGPKSRVTYETVVFHFGLRLLSAVIERHNLK |
| NO: | QQRQTMAYMKRRAAARKKWARSGKKCSRMRNEVEKIKPKWHKDPRWFDIVKEGEP |
| 136 | SIVGISSAGFAIYIVEEPNFPRQDPLEIEYAISIWFRRDRSQYLTFKKIQKAEKLKELQYN |
| | PIPFRLKQEKTSLVFESGDIKFGSRGSIEHFRDEARGKPPKADMDNNRRLTMFSVFSGN |
| | LTNLTEEQYARPVSGLLAPDEKRMPTLLKKLQDFFTPIHEKYGERIKQRLANSEASKRP |
| | FKKLEEYLPAIYLEFRARREGLASNWVLVLINSVRTLVRIKSEDPYIEFKVSQYLLEKED |
| | NKAL |
| SEQ | KQDALFEERLKKAIFIKRQADPLQREELSLLPPNRKIVTGGHESAKDTLKQILRAINGTN |
| ID | QASWNPGTPSGKRDSKSADALAGPKSRVKLETVVFHVGHRLLKKVVEYQGHQKQQH |
| NO: | GLKAFMRTCAAMRKKWKRSGKVVGELREQLANIQPKWHYDSRPLNLCFEGKPSVVG |
| 137 | LRSAGIALYTIQKSVVPVKEPKPIEYAVSIWFRGPKAMDREDRCLEFKKLKIATELRKL |
| | QFEPIVSTLTQGIKGFSLYIQGNSVKFGSRGPIKYFSNESVRQRPPKADPDGNKRLALFS |
| | KFSGDLSDLTEEQWNRPILAFEGIIRRATLGNIQDYLTVGHEQFAISLEQLLSEKESVLQ |
| | MSIEQQRLKKNLGKKAENEWVESFGAEQARKKAQGIREYISGFFQEYCSQREQWAEN |
| | WVQQLNKSVRLFLTIQDSTPFIEFRVARYLPKGEKKKGKAM |
| SEQ | ANHAERHKRLRKEANRAANRNRPLVADCDTGDPLVGICRLLRRGDKMQPNKTGCRS |
| ID | CEQVEPELRDAILVSGPGRLDNYKYELFQRGRAMAVHRLLKRVPKLNRPKKAAGNDE |
| NO: | KKAENKKSEIQKEKQKQRRMMPAVSMKQVSVADFKHVIENTVRHLFGDRRDREIAEC |
| 138 | AALRAASKYFLKSRRVRPRKLPKLANPDHGKELKGLRLREKRAKLKKEKEKQAELAR |
| | SNQKGAVLHVATLKKDAPPMPYEKTQGRNDYTTFVISAAIKVGATRGTKPLLTPQPRE |
| | WQCSLYWRDGQRWIRGGLLGLQAGIVLGPKLNRELLEAVLQRPIECRMSGCGNPLQV |
| | RGAAVDFFMTTNPFYVSGAAYAQKKFKPFGTKRASEDGAAAKAREKLMTQLAKVLD |
| | KVVTQAAHSPLDGIWETRPEAKLRAMIMALEHEWIFLRPGPCHNAAEEVIKCDCTGG |
| | HAILWALIDEARGALEHKEFYAVTRAHTHDCEKQKLGGRLAGFLDLLIAQDVPLDDA |
| | PAARKIKTLLEATPPAPCYKAATSIATCDCEGKFDKLWAIIDATRAGHGTEDLWARTL |
| | AYPQNVNCKCKAGKDLTHRLADFLGLLIKRDGPFRERPPHKVTGDRKLVFSGDKKCK |
| | GHQYVILAKAHNEEVVRAWISRWGLKSRTNKAGYAATELNLLLNWLSICRRRWMD |
| | MLTVQRDTPYIRMKTGRLVVDDKKERKAM |
| SEQ | AKQREALRVALERGIVRASNRTYTLVTNCTKGGPLPEQCRMIERGKARAMKWEPKLV |
| ID | GCGSCAAATVDLPAIEEYAQPGRLDVAKYKLTTQILAMATRRMMVRAAKLSRRKGQ |
| NO: | WPAKVQEEKEEPPEPKKMLKAVEMRPVAIVDFNRVIQTTIEHLWAERANADEAELKA |
| 139 | LKAAAAYFGPSLKIRARGPPKAAIGRELKKAHRKKAYAERKKARRKRAELARSQARG |
| | AAAHAAIRERDIPPMAYERTQGRNDVTTIPIAAAIKIAATRGARPLPAPKPMKWQCSLY |
| | WNEGQRWIRGGMLTAQAYAHAANIHRPMRCEMWGVGNPLKVRAFEGRVADPDGA |
| | KGRKAEFRLQTNAFYVSGAAYRNKKFKPFGTDRGGIGSARKKRERLMAQLAKILDKV |
| | VSQAAHSPLDDIWHTRPAQKLRAMIKQLEHEWMFLRPQAPTVEGTKPDVDVAGNMQ |
| | RQIKALMAPDLPPIEKGSPAKRFTGDKRKKGERAVRVAEAHSDEVVTAWISRWGIQTR |
| | RNEGSYAAQELELLLNWLQICRRRWLDMTAAQRVSPYIRMKSGRMITDAADEGVAPI |
| | PLVENM |
| SEQ | KSISGRSIKHMACLKDMLKSEITEIEEKQKKESLRKWDYYSKFSDEILFRRNLNVSANH |
| ID | DANACYGCNPCAFLKEVYGFRIERRNNERIISYRRGLAGCKSCVQSTGYPPIEFVRRKF |
| NO: | GADKAMEIVREVLHRRNWGALARNIGREKEADPILGELNELLLVDARPYFGNKSAAN |
| 140 | ETNLAFNVITRAAKKFRDEGMYDIHKQLDIHSEEGKVPKGRKSRLIRIERKHKAIHGLD |
| | PGETWRYPHCGKGEKYGVWLNRSRLIHIKGNEYRCLTAFGTTGRRMSLDVACSVLGH |
| | PLVKKKRKKGKKTVDGTELWQIKKATETLPEDPIDCTFYLYAAKPTKDPFILKVGSLK |
| | APRWKKLHKDFFEYSDTEKTQGQEKGKRVVRRGKVPRILSLRPDAKFKVSIWDDPYN |
| | GKNKEGTLLRMELSGLDGAKKPLILKRYGEPNTKPKNFVFWRPHITPHPLTFTPKHDF |
| | GDPNKKTKRRRVFNREYYGHLNDLAKMEPNAKFFEDREVSNKKNPKAKNIRIQAKES |
| | LPNIVAKNGRWAAFDPNDSLWKLYLHWRGRRKTIKGGISQEFQEFKERLDLYKKHED |
| | ESEWKEKEKLWENHEKEWKKTLEIHGSIAEVSQRCVMQSMMGPLDGLVQKKDYVHI |
| | GQSSLKAADDAWTFSANRYKKATGPKWGKISVSNLLYDANQANAELISQSISKYLSK |
| | QKDNQGCEGRKMKFLIKIIEPLRENFVKHTRWLHEMTQKDCEVRAQFSRVSM |
| SEQ | FPSDVGADALKHVRMLQPRLTDEVRKVALTRAPSDRPALARFAAVAQDGLAFVRHL |
| ID | NVSANHDSNCTFPRDPRDPRRGPCEPNPCAFLREVWGFRIVARGNERALSYRRGLAGC |
| NO: | KSCVQSTGFPSVPFHRIGADDCMRKLHEILKARNWRLLARNIGREREADPLLTELSEYL |
| 141 | LVDARTYPDGAAPNSGRLAENVIKRAAKKFRDEGMRDIHAQLRVHSREGKVPKGRLQ |
| | RLRRIERKHRAIHALDPGPSWEAEGSARAEVQGVAVYRSQLLRVGHHTQQIEPVGIVA |
| | RTLFGVGRTDLDVAVSVLGAPLTKRKKGSKTLESTEDFRIAKARETRAEDKIEVAFVL |
| | YPTASLLRDEIPKDAFPAMRIDRFLLKVGSVQADREILLQDDYYRFGDAEVKAGKNKG |
| | RTVTRPVKVPRLQALRPDAKFRVNVWADPFGAGDSPGTLLRLEVSGVTRRSQPLRLL |
| | RYGQPSTQPANFLCWRPHRVPDPMTFTPRQKFGERRKNRRTRRPRVFERLYQVHIKHL |
| | AHLEPNRKWFEEARVSAQKWAKARAIRRKGAEDIPVVAPPAKRRWAALQPNAELWD |
| | LYAHDREARKRFRGGRAAEGEEFKPRLNLYLAHEPEAEWESKRDRWERYEKKWTAV |
| | LEEHSRMCAVADRTLPQFLSDPLGARMDDKDYAFVGKSALAVAEAFVEEGTVERAQ |
| | GNCSITAKKKFASNASRKRLSVANLLDVSDKADRALVFQAVRQYVQRQAENGGVEG |
| | RRMAFLRKLLAPLRQNFVCHTRWLHM |
| SEQ | AARKKKRGKIGITVKAKEKSPPAAGPFMARKLVNVAANVDGVEVHLCVECEADAHG |
| ID | SASARLLGGCRSCTGSIGAEGRLMGSVDVDRERVIAEPVHTETERLGPDVKAFEAGTA |
| NO: | ESKYAIQRGLEYWGVDLISRNRARTVRKMEEADRPESSTMEKTSWDEIAIKTYSQAYH |
| 142 | ASENHLFWERQRRVRQHALALFRRARERNRGESPLQSTQRPAPLVLAALHAEAAAISG |
| | RARAEYVLRGPSANVRAAAADIDAKPLGHYKTPSPKVARGFPVKRDLLRARHRIVGL |
| | SRAYFKPSDVVRGTSDAIAHVAGRNIGVAGGKPKEIEKTFTLPFVAYWEDVDRVVHCS |
| | SFKADGPWVRDQRIKIRGVSSAVGTFSLYGLDVAWSKPTSFYIRCSDIRKKFHPKGFGP |
| | MKHWRQWAKELDRLTEQRASCVVRALQDDEELLQTMERGQRYYDVFSCAATHATR |
| | GEADPSGGCSRCELVSCGVAHKVTKKAKGDTGIEAVAVAGCSLCESKLVGPSKPRVH |
| | RQMAALRQSHALNYLRRLQREWEALEAVQAPTPYLRFKYARHLEVRSM |
| SEQ | AAKKKKQRGKIGISVKPKEGSAPPADGPFMARKLVNVAANVDGVEVNLCIECEADAH |
| ID | GSAPARLLGGCKSCTGSIGAEGRLMGSVDVDRADAIAKPVNTETEKLGPDVQAFEAG |
| NO: | TAETKYALQRGLEYWGVDLISRNRSRTVRRTEEGQPESATMEKTSWDEIAIKSYTRAY |
| 143 | HASENHLFWERQRRVRQHALALFKRAKERNRGDSTLPREPGHGLVAIAALACEAYAV |
| | GGRNLAETVVRGPTFGTARAVRDVEIASLGRYKTPSPKVAHGSPVKRDFLRARHRIVG |
| | LARAYYRPSDVVRGTSDAIAHVAGRNIGVAGGKPRAVEAVFTLPFVAYWEDVDRVV |
| | HCSSFQVSAPWNRDQRMKIAGVTTAAGTFSLHGGELKWAKPTSFYIRCSDTRRKFRP |
| | KGFGPMKRWRQWAKDLDRLVEQRASCVVRALQDDAALLETMERGQRYYDVFACA |
| | VTHATRGEADRLAGCSRCALTPCQEAHRVTTKPRGDAGVEQVQTSDCSLCEGKLVGP |
| | SKPRLHRTLTLLRQEHGLNYLRRLQREWESLEAVQVPTPYLRFKYARHLEVRSM |
| SEQ | TDSQSESVPEVVYALTGGEVPGRVPPDGGSAEGARNAPTGLRKQRGKIKISAKPSKPG |
| ID | SPASSLARTLVNEAANVDGVQSSGCATCRMRANGSAPRALPIGCVACASSIGRAPQEE |
| NO: | TVCALPTTQGPDVRLLEGGHALRKYDIQRALEYWGVDLIGRNLDRQAGRGMEPAEG |
| 144 | ATATMKRVSMDELAVLDFGKSYYASEQHLFAARQRRVRQHAKALKIRAKHANRSGS |
| | VKRALDRSRKQVTALAREFFKPSDVVRGDSDALAHVVGRNLGVSRHPAREIPQTFTLP |
| | LCAYWEDVDRVISCSSLLAGEPFARDQEIRIEGVSSALGSLRLYRGAIEWHKPTSLYIRC |
| | SDTRRKFRPRGGLKKRWRQWAKDLDRLVEQRACCIVRSLQADVELLQTMERAQRFY |
| | DVHDCAATHVGPVAVRCSPCAGKQFDWDRYRLLAALRQEHALNYLRRLQREWESLE |
| | AQQVKMPYLRFKYARKLEVSGPLIGLEVRREPSMGTAIAEM |
| SEQ | AGTAGRRHGSLGARRSINIAGVTDRHGRWGCESCVYTRDQAGNRARCAPCDQSTYAP |
| ID | DVQEVTIGQRQAKYTIFLTLQSFSWTNTMRNNKRAAAGRSKRTTGKRIGQLAEIKITG |
| NO: | VGLAHAHNVIQRSLQHNITKMWRAEKGKSKRVARLKKAKQLTKRRAYFRRRMSRQS |
| 145 | RGNGFFRTGKGGIHAVAPVKIGLDVGMIASGSSEPADEQTVTLDAIWKGRKKKIRLIG |
| | AKGELAVAACRFREQQTKGDKCIPLILQDGEVRWNQNNWQCHPKKLVPLCGLEVSR |
| | KFVSQADRLAQNKVASPLAARFDKTSVKGTLVESDFAAVLVNVTSIYQQCHAMLLRS |
| | QEPTPSLRVQRTITSM |
| SEQ | GVRFSPAQSQVFFRTVIPQSVEARFAINMAAIHDAAGAFGCSVCRFEDRTPRNAKAVH |
| ID | GCSPCTRSTNRPDVFVLPVGAIKAKYDVFMRLLGFNWTHLNRRQAKRVTVRDRIGQL |
| NO: | DELAISMLTGKAKAVLKKSICHNVDKSFKAMRGSLKKLHRKASKTGKSQLRAKLSDL |
| 146 | RERTNTTQEGSHVEGDSDVALNKIGLDVGLVGKPDYPSEESVEVVVCLYFVGKVLILD |
| | AQGRIRDMRAKQYDGFKIPIIQRGQLTVLSVKDLGKWSLVRQDYVLAGDLRFEPKISK |
| | DRKYAECVKRIALITLQASLGFKERIPYYVTKQVEIKNASHIAFVTEAIQNCAENFREM |
| | TEYLMKYQEKSPDLKVLLTQLM |
| SEQ | RAVVGKVFLEQARRALNLATNFGTNHRTGCNGCYVTPGKLSIPQDGEKNAAGCTSCL |
| ID | MKATASYVSYPKPLGEKVAKYSTLDALKGFPWYSLRLNLRPNYRGKPINGVQEVAPV |
| NO: | SKFRLAEEVIQAVQRYHFTELEQSFPGGRRRLRELRAFYTKEYRRAPEQRQHVVNGDR |
| 147 | NIVVVTVLHELGFSVGMFNEVELLPKTPIECAVNVFIRGNRVLLEVRKPQFDKERLLVE |
| | SLWKKDSRRHTAKWTPPNNEGRIFTAEGWKDFQLPLLLGSTSRSLRAIEKEGFVQLAP |
| | GRDPDYNNTIDEQHSGRPFLPLYLYLQGTISQEYCVFAGTWVIPFQDGISPYSTKDTFQ |
| | PDLKRKAYSLLLDAVKHRLGNKVASGLQYGRFPAIEELKRLVRMHGATRKIPRGEKD |
| | LLKKGDPDTPEWWLLEQYPEFWRLCDAAAKRVSQNVGLLLSLKKQPLWQRRWLESR |
| | TRNEPLDNLPLSMALTLHLTNEEAL |
| SEQ | AAVYSKFYIENHFKMGIPETLSRIRGPSIIQGFSVNENYINIAGVGDRDFIFGCKKCKYT |
| ID | RGKPSSKKINKCHPCKRSTYPEPVIDVRGSISEFKYKIYNKLKQEPNQSIKQNTKGRMN |
| NO: | PSDHTSSNDGIIINGIDNRIAYNVIFSSYKHLMEKQINLLRDTTKRKARQIKKYNNSGKK |
| 148 | KHSLRSQTKGNLKNRYHMLGMFKKGSLTITNEGDFITAVRKVGLDISLYKNESLNKQE |
| | VETELCLNIKWGRTKSYTVSGYIPLPINIDWKLYLFEKETGLTLRLFGNKYKIQSKKFLI |
| | AQLFKPKRPPCADPVVKKAQKWSALNAHVQQMAGLFSDSHLLKRELKNRMHKQLDF |
| | KSLWVGTEDYIKWFEELSRSYVEGAEKSLEFFRQDYFCFNYTKQTTM |
| SEQ | PQQQRDLMLMAANYDQDYGNGCGPCTVVASAAYRPDPQAQHGCKRHLRTLGASAV |
| ID | THVGLGDRTATITALHRLRGPAALAARARAAQAASAPMTPDTDAPDDRRRLEAIDAD |
| NO: | DVVLVGAHRALWSAVRRWADDRRAALRRRLHSEREWLLKDQIRWAELYTLIEASGT |
| 149 | PPQGRWRNTLGALRGQSRWRRVLAPTMRATCAETHAELWDALAELVPEMAKDRRG |
| | LLRPPVEADALWRAPMIVEGWRGGHSVVVDAVAPPLDLPQPCAWTAVRLSGDPRQR |
| | WGLHLAVPPLGQVQPPDPLKATLAVSMRHRGGVRVRTLQAMAVDADAPMQRHLQV |
| | PLTLQRGGGLQWGIHSRGVRRREARSMASWEGPPIWTGLQLVNRWKGQGSALLAPD |
| | RPPDTPPYAPDAAVAPAQPDTKRARRTLKEACTVCRCAPGHMRQLQVTLTGDGTWR |
| | RFRLRAPQGAKRKAEVLKVATQHDERIANYTAWYLKRPEHAAGCDTCDGDSRLDGA |
| | CRGCRPLLVGDQCFRRYLDKIEADRDDGLAQIKPKAQEAVAAMAAKRDARAQKVAA |
| | RAAKLSEATGQRTAATRDASHEARAQKELEAVATEGTTVRHDAAAVSAFGSWVARK |
| | GDEYRHQVGVLANRLEHGLRLQELMAPDSVVADQQRASGHARVGYRYVLTAM |
| SEQ | AVAHPVGRGNAGSPGARGPEELPRQLVNRASNVTRPATYGCAPCRHVRLSIPKPVLTG |
| ID | CRACEQTTHPAPKRAVRGGADAAKYDLAAFFAGWAADLEGRNRRRQVHAPLDPQPD |
| NO: | PNHEPAVTLQKIDLAEVSIEEFQRVLARSVKHRHDGRASREREKARAYAQVAKKRRN |
| 150 | SHAHGARTRRAVRRQTRAVRRAHRMGANSGEILVASGAEDPVPEAIDHAAQLRRRIR |
| | ACARDLEGLRHLSRRYLKTLEKPCRRPRAPDLGRARCHALVESLQAAERELEELRRCD |
| | SPDTAMRRLDAVLAAAASTDATFATGWTVVGMDLGVAPRGSAAPEVSPMEMAISVF |
| | WRKGSRRVIVSKPIAGMPIRRHELIRLEGLGTLRLDGNHYTGAGVTKGRGLSEGTEPD |
| | FREKSPSTLGFTLSDYRHESRWRPYGAKQGKTARQFFAAMSRELRALVEHQVLAPMG |
| | PPLLEAHERRFETLLKGQDNKSIHAGGGGRYVWRGPPDSKKRPAADGDWFRFGRGH |
| | ADHRGWANKRHELAANYLQSAFRLWSTLAEAQEPTPYARYKYTRVTM |
| SEQ | WDFLTLQVYERHTSPEVCVAGNSTKCASGTRKSDHTHGVGVKLGAQEINVSANDDR |
| ID | DHEVGCNICVISRVSLDIKGWRYGCESCVQSTPEWRSIVRFDRNHKEAKGECLSRFEY |
| NO: | WGAQSIARSLKRNKLMGGVNLDELAIVQNENVVKTSLKHLFDKRKDRIQANLKAVK |
| 151 | VRMRERRKSGRQRKALRRQCRKLKRYLRSYDPSDIKEGNSCSAFTKLGLDIGISPNKPP |
| | KIEPKVEVVFSLFYQGACDKIVTVSSPESPLPRSWKIKIDGIRALYVKSTKVKFGGRTFR |
| | AGQRNNRRKVRPPNVKKGKRKGSRSQFFNKFAVGLDAVSQQLPIASVQGLWGRAET |
| | KKAQTICLKQLESNKPLKESQRCLFLADNWVVRVCGFLRALSQRQGPTPYIRYRYRCN |
| | M |
| SEQ | ARNVGQRNASRQSKRESAKARSRRVTGGHASVTQGVALINAAANADRDHTTGCEPC |
| ID | TWERVNLPLQEVIHGCDSCTKSSPFWRDIKVVNKGYREAKEEIMRIASGISADHLSRAL |
| NO: | SHNKVMGRLNLDEVCILDFRTVLDTSLKHLTDSRSNGIKEHIRAVHRKIRMRRKSGKT |
| 152 | ARALRKQYFALRRQWKAGHKPNSIREGNSLTALRAVGFDVGVSEGTEPMPAPQTEVV |
| | LSVFYKGSATRILRISSPHPIAKRSWKVKIAGIKALKLIRREHDFSFGRETYNASQRAEK |
| | RKFSPHAARKDFFNSFAVQLDRLAQQLCVSSVENLWVTEPQQKLLTLAKDTAPYGIRE |
| | GARFADTRARLAWNWVFRVCGFTRALHQEQEPTPYCRFTWRSKM |

In some embodiments, the Type V CRISPR/Cas enzyme is a CasΦ nuclease. A CasΦ polypeptide can function as an endonuclease that catalyzes cleavage at a specific sequence in a target nucleic acid. A programmable CasΦ nuclease of the present disclosure may have a single active site in a RuvC domain that is capable of catalyzing pre-crRNA processing and nicking or cleaving of nucleic acids. This compact catalytic site may render the programmable CasΦ nuclease especially advantageous for genome engineering and new functionalities for genome manipulation.

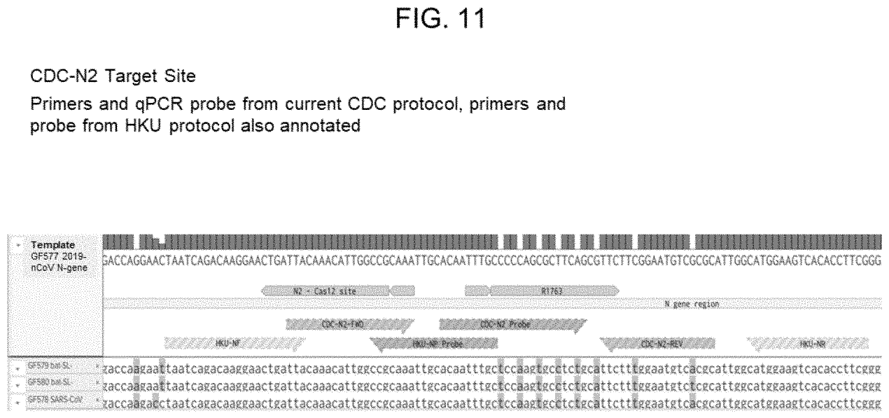

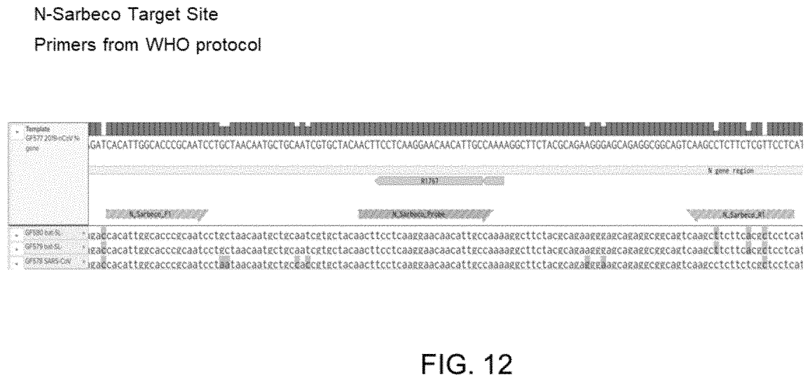

In some embodiments, the RuvC domain is a RuvC-like domain. Various RuvC-like domains are known in the art and are easily identified using online tools such as InterPro (https://www.ebi.ac.uk/interpro/). For example, a RuvC-like domain may be a domain which shares homology with a region of TnpB proteins of the IS605 and other related families of transposons, as described in review articles such as Shmakov et al. (Nature Reviews Microbiology volume 15, pages 169-182(2017)) and Koonin E. V. and Makarova K. S. (2019, Phil. Trans. R. Soc., B 374:20180087). In some embodiments, the RuvC-like domain shares homology with the transposase IS605, OrfB, C-terminal. A transposase IS605, OrfB, C-terminal is easily identified by the skilled person using bioinformatics tools, such as PFAM (Finn et al. (Nucleic Acids Res. 2014 Jan. 1; 42(Database issue): D222-D230); El-Gebali et al. (2019) Nucleic Acids Res. doi:10.1093/nar/gky995). PFAM is a database of protein families in which each entry is composed of a seed alignment which forms the basis to build a profile hidden Markov model (HMM) using the HMMER software (hmmer.org). It is readily accessible via pfam.xfam.org, maintained by EMBL-EBI, which easily allows an amino acid sequence to be analyzed against the current release of PFAM (e.g. version 33.1 from May 2020), but local builds can also be implemented using publicly- and freely-available database files and tools. A transposase IS605, OrfB, C-terminal is easily identified by the skilled person using the HMM PF07282. PF07282 is reproduced for reference in FIG. 11 (accession number PF07282.12). The skilled person would also be able to identify a RuvC domain, for example with the HMM PF18516, using the PFAM tool. PF18516 is reproduced for reference in FIG. 12 (accession number PF18516.2). In some embodiments, the programmable CasΦ nuclease comprises a RuvC-like domain which matches PFAM family PF07282 but does not match PFAM family PF18516, as assessed using the PFAM tool (e.g. using PFAM version 33.1, and the HMM accession numbers PF07282.12 and PF18516.2). PFAM searches should ideally be performed using an E-value cut-off set at 1.0.

TABLE 3 provides amino acid sequences of illustrative CasΦ polypeptides that can be used in compositions and methods of the disclosure.

TABLE 3
CasΦ Amino Acid Sequences

| | SEQ ID | |
|---|---|---|
| Name | NO | Amino Acid Sequence |
| CasΦ.1 | SEQ ID | MADTPTLFTQFLRHHLPGQRFRKDILKQAGRILANKGEDATIA |
| | NO: 221 | FLRGKSEESPPDFQPPVKCPIIACSRPLTEWPIYQASVAIQGYV |
| | | YGQSLAEFEASDPGCSKDGLLGWFDKTGVCTDYFSVQGLNLI |
| | | FQNARKRYIGVQTKVTNRNEKRHKKLKRINAKRIAEGLPELT |
| | | SDEPESALDETGHLIDPPGLNTNIYCYQQVSPKPLALSEVNQL |
| | | PTAYAGYSTSGDDPIQPMVTKDRLSISKGQPGYIPEHQRALLS |
| | | QKKHRRMRGYGLKARALLVIVRIQDDWAVIDLRSLLRNAYW |
| | | RRIVQTKEPSTITKLLKLVTGDPVLDATRMVATFTYKPGIVQV |
| | | RSAKCLKNKQGSKLFSERYLNETVSVTSIDLGSNNLVAVATY |
| | | RLVNGNTPELLQRFTLPSHLVKDFERYKQAHDTLEDSIQKTA |
| | | VASLPQGQQTEIRMWSMYGFREAQERVCQELGLADGSIPWN |
| | | VMTATSTILTDLFLARGGDPKKCMFTSEPKKKKNSKQVLYKI |
| | | RDRAWAKMYRTLLSKETREAWNKALWGLKRGSPDYARLSK |
| | | RKEELARRCVNYTISTAEKRAQCGRTIVALEDLNIGFFHGRGK |
| | | QEPGWVGLFTRKKENRWLMQALHKAFLELAHHRGYHVIEV |
| | | NPAYTSQTCPVCRHCDPDNRDQHNREAFHCIGCGFRGNADL |
| | | DVATHNIAMVAITGESLKRARGSVASKTPQPLAAE |
| CasΦ.2 | SEQ ID | MPKPAVESEFSKVLKKHFPGERFRSSYMKRGGKILAAQGEEA |
| | NO: 222 | VVAYLQGKSEEEPPNFQPPAKCHVVTKSRDFAEWPIMKASEA |
| | | IQRYIYALSTTERAACKPGKSSESHAAWFAATGVSNHGYSHV |
| | | QGLNLIFDHTLGRYDGVLKKVQLRNEKARARLESINASRADE |
| | | GLPEIKAEEEEVATNETGHLLQPPGINPSFYVYQTISPQAYRPR |
| | | DEIVLPPEYAGYVRDPNAPIPLGVVRNRCDIQKGCPGYIPEWQ |
| | | REAGTAISPKTGKAVTVPGLSPKKNKRMRRYWRSEKEKAQD |
| | | ALLVTVRIGTDWVVIDVRGLLRNARWRTIAPKDISLNALLDL |
| | | FTGDPVIDVRRNIVTFTYTLDACGTYARKWTLKGKQTKATLD |
| | | KLTATQTVALVAIDLGQTNPISAGISRVTQENGALQCEPLDRF |
| | | TLPDDLLKDISAYRIAWDRNEEELRARSVEALPEAQQAEVRA |
| | | LDGVSKETARTQLCADFGLDPKRLPWDKMSSNTTFISEALLS |
| | | NSVSRDQVFFTPAPKKGAKKKAPVEVMRKDRTWARAYKPR |
| | | LSVEAQKLKNEALWALKRTSPEYLKLSRRKEELCRRSINYVIE |
| | | KTRRRTQCQIVIPVIEDLNVRFFHGSGKRLPGWDNFFTAKKEN |
| | | RWFIQGLHKAFSDLRTHRSFYVFEVRPERTSITCPKCGHCEVG |
| | | NRDGEAFQCLSCGKTCNADLDVATHNLTQVALTGKTMPKRE |
| | | EPRDAQGTAPARKTKKASKSKAPPAEREDQTPAQEPSQTS |
| CasΦ.3 | SEQ ID | MYILEMADLKSEPSLLAKLLRDRFPGKYWLPKYWKLAEKKR |
| | NO: 223 | LTGGEEAACEYMADKQLDSPPPNFRPPARCVILAKSRPFEDW |
| | | PVHRVASKAQSFVIGLSEQGFAALRAAPPSTADARRDWLRSH |
| | | GASEDDLMALEAQLLETIMGNAISLHGGVLKKIDNANVKAA |
| | | KRLSGRNEARLNKGLQELPPEQEGSAYGADGLLVNPPGLNLN |
| | | IYCRKSCCPKPVKNTARFVGHYPGYLRDSDSILISGTMDRLTII |
| | | EGMPGHIPAWQREQGLVKPGGRRRRLSGSESNMRQKVDPST |
| | | GPRRSTRSGTVNRSNQRTGRNGDPLLVEIRMKEDWVLLDAR |
| | | GLLRNLRWRESKRGLSCDHEDLSLSGLLALFSGDPVIDPVRNE |
| | | VVFLYGEGIIPVRSTKPVGTRQSKKLLERQASMGPLTLISCDL |
| | | GQTNLIAGRASAISLTHGSLGVRSSVRIELDPEIIKSFERLRKDA |
| | | DRLETEILTAAKETLSDEQRGEVNSHEKDSPQTAKASLCRELG |
| | | LHPPSLPWGQMGPSTTFIADMLISHGRDDDAFLSHGEFPTLEK |
| | | RKKFDKRFCLESRPLLSSETRKALNESLWEVKRTSSEYARLSQ |
| | | RKKEMARRAVNFVVEISRRKTGLSNVIVNIEDLNVRIFHGGG |
| | | KQAPGWDGFFRPKSENRWFIQAIHKAFSDLAAHHGIPVIESDP |
| | | QRTSMTCPECGHCDSKNRNGVRFLCKGCGASMDADFDAAC |
| | | RNLERVALTGKPMPKPSTSCERLLSATTGKVCSDHSLSHDAIE |
| | | KAS |
| CasΦ.4 | SEQ ID | MEKEITELTKIRREFPNKKFSSTDMKKAGKLLKAEGPDAVRD |
| | NO: 224 | FLNSCQEIIGDFKPPVKTNIVSISRPFEEWPVSMVGRAIQEYYF |
| | | SLTKEELESVHPGTSSEDHKSFFNITGLSNYNYTSVQGLNLIFK |
| | | NAKAIYDGTLVKANNKNKKLEKKFNEINHKRSLEGLPIITPDF |
| | | EEPFDENGHLNNPPGINRNIYGYQGCAAKVFVPSKHKMVSLP |
| | | KEYEGYNRDPNLSLAGFRNRLEIPEGEPGHVPWFQRMDIPEG |
| | | QIGHVNKIQRFNFVHGKNSGKVKFSDKTGRVKRYHHSKYKD |
| | | ATKPYKFLEESKKVSALDSILAIITIGDDWVVFDIRGLYRNVFY |
| | | RELAQKGLTAVQLLDLFTGDPVIDPKKGVVTFSYKEGVVPVF |
| | | SQKIVPRFKSRDTLEKLTSQGPVALLSVDLGQNEPVAARVCSL |
| | | KNINDKITLDNSCRISFLDDYKKQIKDYRDSLDELEIKIRLEAIN |
| | | SLETNQQVEIRDLDVFSADRAKANTVDMFDIDPNLISWDSMS |
| | | DARVSTQISDLYLKNGGDESRVYFEINNKRIKRSDYNISQLVR |
| | | PKLSDSTRKNLNDSIWKLKRTSEEYLKLSKRKLELSRAVVNY |
| | | TIRQSKLLSGINDIVIILEDLDVKKKFNGRGIRDIGWDNFFSSR |
| | | KENRWFIPAFHKAFSELSSNRGLCVIEVNPAWTSATCPDCGFC |
| | | SKENRDGINFTCRKCGVSYHADIDVATLNIARVAVLGKPMSG |
| | | PADRERLGDTKKPRVARSRKTMKRKDISNSTVEAMVTA |
| CasΦ.5 | SEQ ID | MDMLDTETNYATETPAQQQDYSPKPPKKAQRAPKGFSKKAR |
| | NO: 225 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEK |
| | | ILWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADI |
| | | RGALRNAQWRKLLKPDATYQSLFNLFTGDPVVNTRTNHLTM |
| | | AYREGVVNIVKSRSFKGRQTREHLLTLLGQGKTVAGVSFDLG |
| | | QKHAAGLLAAHFGLGEDGNPVFTPIQACFLPQRYLDSLTNYR |
| | | NRYDALTLDMRRQSLLALTPAQQQEFADAQRDPGGQAKRAC |
| | | CLKLNLNPDEIRWDLVSGISTMISDLYIERGGDPRDVHQQVET |
| | | KPKGKRKSEIRILKIRDGKWAYDFRPKIADETRKAQREQLWK |
| | | LQKASSEFERLSRYKINIARAIANWALQWGRELSGCDIVIPVL |
| | | EDLNVGSKFFDGKGKWLLGWDNRFTPKKENRWFIKVLHKA |
| | | VAELAPHRGVPVYEVMPHRTSMTCPACHYCHPTNREGDRFE |
| | | CQSCHVVKNTDRDVAPYNILRVAVEGKTLDRWQAEKKPQAE |
| | | PDRPMILIDNQES |
| CasΦ.6 | SEQ ID | MDMLDTETNYATETPAQQQDYSPKPPKKAQRAPKGFSKKAR |
| | NO: 226 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEK |
| | | ILWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADI |
| | | RGALRNAQWRKLLKPDATYQSLFNLFTGDPVVNTRTNHLTM |
| | | AYREGVVDIVKSRSFKGRQTREHLLTLLGQGKTVAGVSFDLG |
| | | QKHAAGLLAAHFGLGEDGNPVFTPIQACFLPQRYLDSLTNYR |
| | | NRYDALTLDMRRQSLLALTPAQQQEFADAQRDPGGQAKRAC |
| | | CLKLNLNPDEIRWDLVSGISTMISDLYIERGGDPRDVHQQVET |
| | | KPKGKRKSEIRILKIRDGKWAYDFRPKIADETRKAQREQLWK |
| | | LQKASSEFERLSRYKINIARAIANWALQWGRELSGCDIVIPVL |
| | | EDLNVGSKFFDGKGKWLLGWDNRFTPKKENRWFIKVLHKA |
| | | VAELAPHKGVPVYEVMPHRTSMTCPACHYCHPTNREGDRFE |
| | | CQSCHVVKNTDRDVAPYNILRVAVEGKTLDRWQAEKKPQAE |
| | | PDRPMILIDNQES |
| CasΦ.7 | SEQ ID | MSSLPTPLELLKQKHADLFKGLQFSSKDNKMAGKVLKKDGE |
| | NO: 227 | EAALAFLSERGVSRGELPNFRPPAKTLVVAQSRPFEEFPIYRVS |
| | | EAIQLYVYSLSVKELETVPSGSSTKKEHQRFFQDSSVPDFGYT |
| | | SVQGLNKIFGLARGIYLGVITRGENQLQKAKSKHEALNKKRR |
| | | ASGEAETEFDPTPYEYMTPERKLAKPPGVNHSIMCYVDISVDE |
| | | FDFRNPDGIVLPSEYAGYCREINTAIEKGTVDRLGHLKGGPGY |
| | | IPGHQRKESTTEGPKINFRKGRIRRSYTALYAKRDSRRVRQGK |
| | | LALPSYRHHMMRLNSNAESAILAVIFFGKDWVVFDLRGLLRN |
| | | VRWRNLFVDGSTPSTLLGMFGDPVIDPKRGVVAFCYKEQIVP |
| | | VVSKSITKMVKAPELLNKLYLKSEDPLVLVAIDLGQTNPVGV |
| | | GVYRVMNASLDYEVVTRFALESELLREIESYRQRTNAFEAQI |
| | | RAETFDAMTSEEQEEITRVRAFSASKAKENVCHRFGMPVDAV |
| | | DWATMGSNTIHIAKWVMRHGDPSLVEVLEYRKDNEIKLDKN |
| | | GVPKKVKLTDKRIANLTSIRLRFSQETSKHYNDTMWELRRKH |
| | | PVYQKLSKSKADFSRRVVNSIIRRVNHLVPRARIVFIIEDLKNL |
| | | GKVFHGSGKRELGWDSYFEPKSENRWFIQVLHKAFSETGKH |
| | | KGYYIIECWPNWTSCTCPKCSCCDSENRHGEVFRCLACGYTC |
| | | NTDFGTAPDNLVKIATTGKGLPGPKKRCKGSSKGKNPKIARS |
| | | SETGVSVTESGAPKVKKSSPTQTSQSSSQSAP |
| CasΦ.8 | SEQ ID | MNKIEKEKTPLAKLMNENFAGLRFPFAIIKQAGKKLLKEGEL |
| | NO: 228 | KTIEYMTGKGSIEPLPNFKPPVKCLIVAKRRDLKYFPICKASCE |
| | | IQSYVYSLNYKDFMDYFSTPMTSQKQHEEFFKKSGLNIEYQN |
| | | VAGLNLIFNNVKNTYNGVILKVKNRNEKLKKKAIKNNYEFEE |
| | | IKTFNDDGCLINKPGINNVIYCFQSISPKILKNITHLPKEYNDYD |
| | | CSVDRNIIQKYVSRLDIPESQPGHVPEWQRKLPEFNNTNNPRR |
| | | RRKWYSNGRNISKGYSVDQVNQAKIEDSLLAQIKIGEDWIILD |
| | | IRGLLRDLNRRELISYKNKLTIKDVLGFFSDYPIIDIKKNLVTFC |
| | | YKEGVIQVVSQKSIGNKKSKQLLEKLIENKPIALVSIDLGQTNP |
| | | VSVKISKLNKINNKISIESFTYRFLNEEILKEIEKYRKDYDKLEL |
| | | KLINEA |
| CasΦ.9 | SEQ ID | MDMLDTETNYATETPSQQQDYSPKPPKKDRRAPKGFSKKAR |
| | NO: 229 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEK |
| | | ILWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADI |
| | | RGALRNAQWRKLLKPDATYQSLFNLFTGDPVVNTRTNHLTM |
| | | AYREGVVDIVKSRSFKGRQTREHLLTLLGQGKTVAGVSFDLG |
| | | QKHAAGLLAAHFGLGEDGNPVFTPIQACFLPQRYLDSLTNYR |
| | | NRYDALTLDMRRQSLLALTPAQQQEFADAQRDPGGQAKRAC |
| | | CLKLNLNPDEIRWDLVSGISTMISDLYIERGGDPRDVHQQVET |
| | | KPKGKRKSEIRILKIRDGKWAYDFRPKIADETRKAQREQLWK |
| | | LQKASSEFERLSRYKINIARAIANWALQWGRELSGCDIVIPVL |
| | | EDLNVGSKFFDGKGKWLLGWDNRFTPKKENRWFIKVLHKA |
| | | VAELAPHRGVPVYEVMPHRTSMTCPACHYCHPTNREGDRFE |
| | | CQSCHVVKNTDRDVAPYNILRVAVEGKTLDRWQAEKKPQAE |
| | | PDRPMILIDNQES |
| CasΦ.10 | SEQ ID | MDMLDTETNYATETPSQQQDYSPKPPKKDRRAPKGFSKKAR |
| | NO: 230 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEK |
| | | ILWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADI |
| | | RGALRNAQWRKLLKPDATYQSLFNLFTGDPVVNTRTNHLTM |
| | | AYREGVVNIVKSRSFKGRQTREHLLTLLGQGKTVAGVSFDLG |
| | | QKHAAGLLAAHFGLGEDGNPVFTPIQACFLPQRYLDSLTNYR |
| | | NRYDALTLDMRRQSLLALTPAQQQEFADAQRDPGGQAKRAC |
| | | CLKLNLNPDEIRWDLVSGISTMISDLYIERGGDPRDVHQQVET |
| | | KPKGKRKSEIRILKIRDGKWAYDFRPKIADETRKAQREQLWK |
| | | LQKASSEFERLSRYKINIARAIANWALQWGRELSGCDIVIPVL |
| | | EDLNVGSKFFDGKGKWLLGWDNRFTPKKENRWFIKVLHKA |
| | | VAELAPHRGVPVYEVMPHRTSMTCPACHYCHPTNREGDRFE |
| | | CQSCHVVKNTDRDVAPYNILRVAVEGKTLDRWQAEKKPQAE |
| | | PDRPMILIDNQES |
| CasΦ.11 | SEQ ID | MSNKTTPPSPLSLLLRAHFPGLKFESQDYKIAGKKLRDGGPEA |
| | NO: 231 | VISYLTGKGQAKLKDVKPPAKAFVIAQSRPFIEWDLVRVSRQI |
| | | QEKIFGIPATKGRPKQDGLSETAFNEAVASLEVDGKSKLNEET |
| | | RAAFYEVLGLDAPSLHAQAQNALIKSAISIREGVLKKVENRNE |
| | | KNLSKTKRRKEAGEEATFVEEKAHDERGYLIHPPGVNQTIPG |
| | | YQAVVIKSCPSDFIGLPSGCLAKESAEALTDYLPHDRMTIPKG |
| | | QPGYVPEWQHPLLNRRKNRRRRDWYSASLNKPKATCSKRSG |
| | | TPNRKNSRTDQIQSGRFKGAIPVLMRFQDEWVIIDIRGLLRNA |
| | | RYRKLLKEKSTIPDLLSLFTGDPSIDMRQGVCTFIYKAGQACS |
| | | AKMVKTKNAPEILSELTKSGPVVLVSIDLGQTNPIAAKVSRVT |
| | | QLSDGQLSHETLLRELLSNDSSDGKEIARYRVASDRLRDKLA |
| | | NLAVERLSPEHKSEILRAKNDTPALCKARVCAALGLNPEMIA |
| | | WDKMTPYTEFLATAYLEKGGDRKVATLKPKNRPEMLRRDIK |
| | | FKGTEGVRIEVSPEAAEAYREAQWDLQRTSPEYLRLSTWKQE |
| | | LTKRILNQLRHKAAKSSQCEVVVMAFEDLNIKMMHGNGKW |
| | | ADGGWDAFFIKKRENRWFMQAFHKSLTELGAHKGVPTIEVT |
| | | PHRTSITCTKCGHCDKANRDGERFACQKCGFVAHADLEIATD |
| | | NIERVALTGKPMPKPESERSGDAKKSVGARKAAFKPEEDAEA |
| | | AE |
| CasΦ.12 | SEQ ID | MIKPTVSQFLTPGFKLIRNHSRTAGLKLKNEGEEACKKFVREN |
| | NO: 232 | EIPKDECPNFQGGPAIANIIAKSREFTEWEIYQSSLAIQEVIFTLP |
| | | KDKLPEPILKEEWRAQWLSEHGLDTVPYKEAAGLNLIIKNAV |
| | | NTYKGVQVKVDNKNKNNLAKINRKNEIAKLNGEQEISFEEIK |
| | | AFDDKGYLLQKPSPNKSIYCYQSVSPKPFITSKYHNVNLPEEYI |
| | | GYYRKSNEPIVSPYQFDRLRIPIGEPGYVPKWQYTFLSKKENK |
| | | RRKLSKRIKNVSPILGIICIKKDWCVFDMRGLLRTNHWKKYH |
| | | KPTDSINDLFDYFTGDPVIDTKANVVRFRYKMENGIVNYKPV |
| | | REKKGKELLENICDQNGSCKLATVDVGQNNPVAIGLFELKKV |
| | | NGELTKTLISRHPTPIDFCNKITAYRERYDKLESSIKLDAIKQLT |
| | | SEQKIEVDNYNNNFTPQNTKQIVCSKLNINPNDLPWDKMISGT |
| | | HFISEKAQVSNKSEIYFTSTDKGKTKDVMKSDYKWFQDYKPK |
| | | LSKEVRDALSDIEWRLRRESLEFNKLSKSREQDARQLANWISS |
| | | MCDVIGIENLVKKNNFFGGSGKREPGWDNFYKPKKENRWWI |
| | | NAIHKALTELSQNKGKRVILLPAMRTSITCPKCKYCDSKNRN |
| | | GEKFNCLKCGIELNADIDVATENLATVAITAQSMPKPTCERSG |
| | | DAKKPVRARKAKAPEFHDKLAPSYTVVLREAV |
| CasΦ.13 | SEQ ID | MRQPAEKTAFQVFRQEVIGTQKLSGGDAKTAGRLYKQGKME |
| | NO: 233 | AAREWLLKGARDDVPPNFQPPAKCLVVAVSHPFEEWDISKT |
| | | NHDVQAYIYAQPLQAEGHLNGLSEKWEDTSADQHKLWFEKT |
| | | GVPDRGLPVQAINKIAKAAVNRAFGVVRKVENRNEKRRSRD |
| | | NRIAEHNRENGLTEVVREAPEVATNADGFLLHPPGIDPSILSY |
| | | ASVSPVPYNSSKHSFVRLPEEYQAYNVEPDAPIPQFVVEDRFA |
| | | IPPGQPGYVPEWQRLKCSTNKHRRMRQWSNQDYKPKAGRR |
| | | AKPLEFQAHLTRERAKGALLVVMRIKEDWVVFDVRGLLRNV |
| | | EWRKVLSEEAREKLTLKGLLDLFTGDPVIDTKRGIVTFLYKAE |
| | | ITKILSKRTVKTKNARDLLLRLTEPGEDGLRREVGLVAVDLG |
| | | QTHPIAAAIYRIGRTSAGALESTVLHRQGLREDQKEKLKEYRK |
| | | RHTALDSRLRKEAFETLSVEQQKEIVTVSGSGAQITKDKVCN |
| | | YLGVDPSTLPWEKMGSYTHFISDDFLRRGGDPNIVHFDRQPK |
| | | KGKVSKKSQRIKRSDSQWVGRMRPRLSQETAKARMEADWA |
| | | AQNENEEYKRLARSKQELARWCVNTLLQNTRCITQCDEIVVV |
| | | IEDLNVKSLHGKGAREPGWDNFFTPKTENRWFIQILHKTFSEL |
| | | PKHRGEHVIEGCPLRTSITCPACSYCDKNSRNGEKFVCVACG |
| | | ATFHADFEVATYNLVRLATTGMPMPKSLERQGGGEKAGGAR |
| | | KARKKAKQVEKIVVQANANVTMNGASLHSP |
| CasΦ.14 | SEQ ID | MSSLPTPLELLKQKHADLFKGLQFSSKDNKMAGKVLKKDGE |
| | NO: 234 | EAALAFLSERGVSRGELPNFRPPAKTLVVAQSRPFEEFPIYRVS |
| | | EAIQLYVYSLSVKELETVPSGSSTKKEHQRFFQDSSVPDFGYT |
| | | SVQGLNKIFGLARGIYLGVITRGENQLQKAKSKHEALNKKRR |
| | | ASGEAETEFDPTPYEYMTPERKLAKPPGVNHSIMCYVDISVDE |
| | | FDFRNPDGIVLPSEYAGYCREINTAIEKGTVDRLGHLKGGPGY |
| | | IPGHQRKESTTEGPKINFRKGRIRRSYTALYAKRDSRRVRQGK |
| | | LALPSYRHHMMRLNSNAESAILAVIFFGKDWVVFDLRGLLRN |
| | | VRWRNLFVDGSTPSTLLGMFGDPVIDPKRGVVAFCYKEQIVP |
| | | VVSKSITKMVKAPELLNKLYLKSEDPLVLVAIDLGQTNPVGV |
| | | GVYRVMNASLDYEVVTRFALESELLREIESYRQRTNAFEAQI |
| | | RAETFDAMTSEEQEEITRVRAFSASKAKENVCHRFGMPVDAV |
| | | DWATMGSNTIHIAKWVMRHGDPSLVEVLEYRKDNEIKLDKN |
| | | GVPKKVKLTDKRIANLTSIRLRFSQETSKHYNDTMWELRRKH |
| | | PVYQKLSKSKADFSRRVVNSIIRRVNHLVPRARIVFIIEDLKNL |
| | | GKVFHGSGKRELGWDSYFEPKSENRWFIQVLHKAFSETGKH |
| | | KGYYIIECWPNWTSCTCPKCSCCDSENRHGEVFRCLACGYTC |
| | | NTDFGTAPDNLVKIATTGKGLPGPKKRCKGSSKGKNPKIARS |
| | | SETGVSVTESGAPKVKKSSPTQTSQSSSQSAP |
| CasΦ.15 | SEQ ID | MIKPTVSQFLTPGFKLIRNHSRTAGLKLKNEGEEACKKFVREN |
| | NO: 235 | EIPKDECPNFQGGPAIANIIAKSREFTEWEIYQSSLAIQEVIFTLP |
| | | KDKLPEPILKEEWRAQWLSEHGLDTVPYKEAAGLNLIIKNAV |
| | | NTYKGVQVKVDNKNKNNLAKINRKNEIAKLNGEQEISFEEIK |
| | | AFDDKGYLLQKPSPNKSIYCYQSVSPKPFITSKYHNVNLPEEYI |
| | | GYYRKSNEPIVSPYQFDRLRIPIGEPGYVPKWQYTFLSKKENK |
| | | RRKLSKRIKNVSPILGIICIKKDWCVFDMRGLLRTNHWKKYH |
| | | KPTDSINDLFDYFTGDPVIDTKANVVRFRYKMENGIVNYKPV |
| | | REKKGKELLENICDQNGSCKLATVDVGQNNPVAIGLFELKKV |
| | | NGELTKTLISRHPTPIDFCNKITAYRERYDKLESSIKLDAIKQLT |
| | | SEQKIEVDNYNNNFTPQNTKQIVCSKLNINPNDLPWDKMISGT |
| | | HFISEKAQVSNKSEIYFTSTDKGKTKDVMKSDYKWFQDYKPK |
| | | LSKEVRDALSDIEWRLRRESLEFNKLSKSREQDARQLANWISS |
| | | MCDVIGIENLVKKNNFFGGSGKREPGWDNFYKPKKENRWWI |
| | | NAIHKALTELSQNKGKRVILLPAMRTSITCPKCKYCDSKNRN |
| | | GEKFNCLKCGIELNADIDVATENLATVAITAQSMPKPTCERSG |
| | | DAKKPVRARKAKAPEFHDKLAPSYTVVLREAV |
| CasΦ.16 | SEQ ID | MSNKTTPPSPLSLLLRAHFPGLKFESQDYKIAGKKLRDGGPEA |
| | NO: 236 | VISYLTGKGQAKLKDVKPPAKAFVIAQSRPFIEWDLVRVSRQI |
| | | QEKIFGIPATKGRPKQDGLSETAFNEAVASLEVDGKSKLNEET |
| | | RAAFYEVLGLDAPSLHAQAQNALIKSAISIREGVLKKVENRNE |
| | | KNLSKTKRRKEAGEEATFVEEKAHDERGYLIHPPGVNQTIPG |
| | | YQAVVIKSCPSDFIGLPSGCLAKESAEALTDYLPHDRMTIPKG |
| | | QPGYVPEWQHPLLNRRKNRRRRDWYSASLNKPKATCSKRSG |
| | | TPNRKNSRTDQIQSGRFKGAIPVLMRFQDEWVIIDIRGLLRNA |
| | | RYRKLLKEKSTIPDLLSLFTGDPSIDMRQGVCTFIYKAGQACS |
| | | AKMVKTKNAPEILSELTKSGPVVLVSIDLGQTNPIAAKVSRVT |
| | | QLSDGQLSHETLLRELLSNDSSDGKEIARYRVASDRLRDKLA |
| | | NLAVERLSPEHKSEILRAKNDTPALCKARVCAALGLNPEMIA |
| | | WDKMTPYTEFLATAYLEKGGDRKVATLKPKNRPEMLRRDIK |
| | | FKGTEGVRIEVSPEAAEAYREAQWDLQRTSPEYLRLSTWKQE |
| | | LTKRILNQLRHKAAKSSQCEVVVMAFEDLNIKMMHGNGKW |
| | | ADGGWDAFFIKKRENRWFMQAFHKSLTELGAHKGVPTIEVT |
| | | PHRTSITCTKCGHCDKANRDGERFACQKCGFVAHADLEIATD |
| | | NIERVALTGKPMPKPESERSGDAKKSVGARKAAFKPEEDAEA |
| | | AE |
| CasΦ.17 | SEQ ID | MYSLEMADLKSEPSLLAKLLRDRFPGKYWLPKYWKLAEKKR |
| | NO: 237 | LTGGEEAACEYMADKQLDSPPPNFRPPARCVILAKSRPFEDW |
| | | PVHRVASKAQSFVIGLSEQGFAALRAAPPSTADARRDWLRSH |
| | | GASEDDLMALEAQLLETIMGNAISLHGGVLKKIDNANVKAA |
| | | KRLSGRNEARLNKGLQELPPEQEGSAYGADGLLVNPPGLNLN |
| | | IYCRKSCCPKPVKNTARFVGHYPGYLRDSDSILISGTMDRLTII |
| | | EGMPGHIPAWQREQGLVKPGGRRRRLSGSESNMRQKVDPST |
| | | GPRRSTRSGTVNRSNQRTGRNGDPLLVEIRMKEDWVLLDAR |
| | | GLLRNLRWRESKRGLSCDHEDLSLSGLLALFSGDPVIDPVRNE |
| | | VVFLYGEGIIPVRSTKPVGTRQSKKLLERQASMGPLTLISCDL |
| | | GQTNLIAGRASAISLTHGSLGVRSSVRIELDPEIIKSFERLRKDA |
| | | DRLETEILTAAKETLSDEQRGEVNSHEKDSPQTAKASLCRELG |
| | | LHPPSLPWGQMGPSTTFIADMLISHGRDDDAFLSHGEFPTLEK |
| | | RKKFDKRFCLESRPLLSSETRKALNESLWEVKRTSSEYARLSQ |
| | | RKKEMARRAVNFVVEISRRKTGLSNVIVNIEDLNVRIFHGGG |
| | | KQAPGWDGFFRPKSENRWFIQAIHKAFSDLAAHHGIPVIESDP |
| | | QRTSMTCPECGHCDSKNRNGVRFLCKGCGASMDADFDAAC |
| | | RNLERVALTGKPMPKPSTSCERLLSATTGKVCSDHSLSHDAIE |
| | | KAS |
| CasΦ.18 | SEQ ID | MEKEITELTKIRREFPNKKFSSTDMKKAGKLLKAEGPDAVRD |
| | NO: 238 | FLNSCQEIIGDFKPPVKTNIVSISRPFEEWPVSMVGRAIQEYYF |
| | | SLTKEELESVHPGTSSEDHKSFFNITGLSNYNYTSVQGLNLIFK |
| | | NAKAIYDGTLVKANNKNKKLEKKFNEINHKRSLEGLPIITPDF |
| | | EEPFDENGHLNNPPGINRNIYGYQGCAAKVFVPSKHKMVSLP |
| | | KEYEGYNRDPNLSLAGFRNRLEIPEGEPGHVPWFQRMDIPEG |
| | | QIGHVNKIQRFNFVHGKNSGKVKFSDKTGRVKRYHHSKYKD |
| | | ATKPYKFLEESKKVSALDSILAIITIGDDWVVFDIRGLYRNVFY |
| | | RELAQKGLTAVQLLDLFTGDPVIDPKKGVVTFSYKEGVVPVF |
| | | SQKIVPRFKSRDTLEKLTSQGPVALLSVDLGQNEPVAARVCSL |
| | | KNINDKITLDNSCRISFLDDYKKQIKDYRDSLDELEIKIRLEAIN |
| | | SLETNQQVEIRDLDVFSADRAKANTVDMFDIDPNLISWDSMS |
| | | DARVSTQISDLYLKNGGDESRVYFEINNKRIKRSDYNISQLVR |
| | | PKLSDSTRKNLNDSIWKLKRTSEEYLKLSKRKLELSRAVVNY |
| | | TIRQSKLLSGINDIVIILEDLDVKKKFNGRGIRDIGWDNFFSSR |
| | | KENRWFIPAFHKTFSELSSNRGLCVIEVNPAWTSATCPDCGFC |
| | | SKENRDGINFTCRKCGVSYHADIDVATLNIARVAVLGKPMSG |
| | | PADRERLGDTKKPRVARSRKTMKRKDISNSTVEAMVTA |
| CasΦ.19 | SEQ ID | MLVRTSTLVQDNKNSRSASRAFLKKPKMPKNKHIKEPTELAK |
| | NO: 239 | LIRELFPGQRFTRAINTQAGKILKHKGRDEVVEFLKNKGIDKE |
| | | QFMDFRPPTKARIVATSGAIEEFSYLRVSMAIQECCFGKYKFP |
| | | KEKVNGKLVLETVGLTKEELDDFLPKKYYENKKSRDRFFLKT |
| | | GICDYGYTYAQGLNEIFRNTRAIYEGVFTKVNNRNEKRREKK |
| | | DKYNEERRSKGLSEEPYDEDESATDESGHLINPPGVNLNIWTC |
| | | EGFCKGPYVTKLSGTPGYEVILPKVFDGYNRDPNEIISCGITDR |
| | | FAIPEGEPGHIPWHQRLEIPEGQPGYVPGHQRFADTGQNNSGK |
| | | ANPNKKGRMRKYYGHGTKYTQPGEYQEVFRKGHREGNKRR |
| | | YWEEDFRSEAHDCILYVIHIGDDWVVCDLRGPLRDAYRRGL |
| | | VPKEGITTQELCNLFSGDPVIDPKHGVVTFCYKNGLVRAQKTI |
| | | SAGKKSRELLGALTSQGPIALIGVDLGQTEPVGARAFIVNQAR |
| | | GSLSLPTLKGSFLLTAENSSSWNVFKGEIKAYREAIDDLAIRLK |
| | | KEAVATLSVEQQTEIESYEAFSAEDAKQLACEKFGVDSSFILW |
| | | EDMTPYHTGPATYYFAKQFLKKNGGNKSLIEYIPYQKKKSKK |
| | | TPKAVLRSDYNIACCVRPKLLPETRKALNEAIRIVQKNSDEYQ |
| | | RLSKRKLEFCRRVVNYLVRKAKKLTGLERVIIAIEDLKSLEKF |
| | | FTGSGKRDNGWSNFFRPKKENRWFIPAFHKAFSELAPNRGFY |
| | | VIECNPARTSITDPDCGYCDGDNRDGIKFECKKCGAKHHTDL |
| | | DVAPLNIAIVAVTGRPMPKTVSNKSKRERSGGEKSVGASRKR |
| | | NHRKSKANQEMLDATSSAAE |
| CasΦ.20 | SEQ ID | MPKIKKPTEISLLRKEVFPDLHFAKDRMRAASLVLKNEGREA |
| | NO: 240 | AIEYLRVNHEDKPPNFMPPAKTPYVALSRPLEQWPIAQASIAI |
| | | QKYIFGLTKDEFSATKKLLYGDKSTPNTESRKRWFEVTGVPN |
| | | FGYMSAQGLNAIFSGALARYEGVVQKVENRNKKRFEKLSEK |
| | | NQLLIEEGQPVKDYVPDTAYHTPETLQKLAENNHVRVEDLG |
| | | DMIDRLVHPPGIHRSIYGYQQVPPFAYDPDNPKGIILPKAYAG |
| | | YTRKPHDIIEAMPNRLNIPEGQAGYIPEHQRDKLKKGGRVKR |
| | | LRTTRVRVDATETVRAKAEALNAEKARLRGKEAILAVFQIEE |
| | | DWALIDMRGLLRNVYMRKLIAAGELTPTTLLGYFTETLTLDP |
| | | RRTEATFCYHLRSEGALHAEYVRHGKNTRELLLDLTKDNEKI |
| | | ALVTIDLGQRNPLAAAIFRVGRDASGDLTENSLEPVSRMLLPQ |
| | | AYLDQIKAYRDAYDSFRQNIWDTALASLTPEQQRQILAYEAY |
| | | TPDDSKENVLRLLLGGNVMPDDLPWEDMTKNTHYISDRYLA |
| | | DGGDPSKVWFVPGPRKRKKNAPPLKKPPKPRELVKRSDHNIS |
| | | HLSEFRPQLLKETRDAFEKAKIDTERGHVGYQKLSTRKDQLC |
| | | KEILNWLEAEAVRLTRCKTMVLGLEDLNGPFFNQGKGKVRG |
| | | WVSFFRQKQENRWIVNGFRKNALARAHDKGKYILELWPSWT |
| | | SQTCPKCKHVHADNRHGDDFVCLQCGARLHADAEVATWNL |
| | | AVVAIQGHSLPGPVREKSNDRKKSGSARKSKKANESGKVVG |
| | | AWAAQATPKRATSKKETGTARNPVYNPLETQASCPAP |
| CasΦ.21 | SEQ ID | MTPSPQIARLVETPLAAALKAHHPGKKFRSDYLKKAGKILKD |
| | NO: 241 | QGVEAAMAHLDGKDQAEPPNFKPPAKCRIVARSREFSEWPIV |
| | | KASVEIQKYIYGLTLEERKACDPGKSSASHKAWFAKTGVNTF |
| | | GYSSVQGFNLIFGHTLGRYDGVLVKTENLNKKRAEKNERFRA |
| | | KALAEGRAEPVCPPLVTATNDTGQDVTLEDGRVVRPGQLLQ |
| | | PPGINPNIYAYQQVSPKAYVPGIIELPEEFQGYSRDPNAVILPL |
| | | VPRDRLSIPKGQPGYVPEPHREGLTGRKDRRMRRYYETERGT |
| | | KLKRPPLTAKGRADKANEALLVVVRIDSDWVVMDVRGLLR |
| | | NARWRRLVSKEGITLNGLLDLFTGDPVLNPKDCSVSRDTGDP |
| | | VNDPRHGVVTFCYKLGVVDVCSKDRPIKGFRTKEVLERLTSS |
| | | GTVGMVSIDLGQTNPVAAAVSRVTKGLQAETLETFTLPDDLL |
| | | GKVRAYRAKTDRMEEGFRRNALRKLTAEQQAEITRYNDATE |
| | | QQAKALVCSTYGIGPEEVPWERMTSNTTYISDHILDHGGDPD |
| | | TVFFMATKRGQNKPTLHKRKDKAWGQKFRPAISVETRLARQ |
| | | AAEWELRRASLEFQKLSVWKTELCRQAVNYVMERTKKRTQ |
| | | CDVIIPVIEDLPVPLFHGSGKRDPGWANFFVHKRENRWFIDGL |
| | | HKAFSELGKHRGIYVFEVCPQRTSITCPKCGHCDPDNRDGEKF |
| | | VCLSCQATLNADLDVATTNLVRVALTGKVMPRSERSGDAQT |
| | | PGPARKARTGKIKGSKPTSAPQGATQTDAKAHLSQTGV |
| CasΦ.22 | SEQ ID | MTPSPQIARLVETPLAAALKAHHPGKKFRSDYLKKAGKILKD |
| | NO: 242 | QGVEAAMAHLDGKDQAEPPNFKPPAKCRIVARSREFSEWPIV |
| | | KASVEIQKYIYGLTLEERKACDPGKSSASHKAWFAKTGVNTF |
| | | GYSSVQGFNLIFGHTLGRYDGVLVKTENLNKKRAEKNERFRA |
| | | KALAEGRAEPVCPPLVTATNDTGQDVTLEDGRVVRPGQLLQ |
| | | PPGINPNIYAYQQVSPKAYVPGIIELPEEFQGYSRDPNAVILPL |
| | | VPRDRLSIPKGQPGYVPEPHREGLTGRKDRRMRRYYETERGT |
| | | KLKRPPLTAKGRADKANEALLVVVRIDSDWVVMDVRGLLR |
| | | NARWRRLVSKEGITLNGLLDLFTGDPVLNPKDCSVSRDTGDP |
| | | VNDPRHGVVTFCYKLGVVDVCSKDRPIKGFRTKEVLERLTSS |
| | | GTVGMVSIDLGQTNPVAAAVSRVTKGLQAETLETFTLPDDLL |
| | | GKVRAYRAKTDRMEEGFRRNALRKLTAEQQAEITRYNDATE |
| | | QQAKALVCSTYGIGPEEVPWERMTSNTTYISDHILDHGGDPD |
| | | TVFFMATKRGQNKPTLHKRKDKAWGQKFRPAISVETRLARQ |
| | | AAEWELRRASLEFQKLSVWKTELCRQAVNYVMERTKKRTQ |
| | | CDVIIPVIEDLPVPLFHGSGKRDPGWANFFVHKRENRWFIDGL |
| | | HKAFSELGKHRGIYVFEVCPQRTSITCPKCGHCDPDNRDGEKF |
| | | VCLSCQATLHADLDVATTNLVRVALTGKVMPRSERSGDAQT |
| | | PGPARKARTGKIKGSKPTSAPQGATQTDAKAHLSQTGV |
| CasΦ.23 | SEQ ID | MKTEKPKTALTLLREEVFPGKKYRLDVLKEAGKKLSTKGRE |
| | NO: 243 | ATIEFLTGKDEERPQNFQPPAKTSIVAQSRPFDQWPIVQVSLA |
| | | VQKYIYGLTQSEFEANKKALYGETGKAISTESRRAWFEATGV |
| | | DNFGFTAAQGINPIFSQAVARYEGVIKKVENRNEKKLKKLTK |
| | | KNLLRLESGEEIEDFEPEATFNEEGRLLQPPGANPNIYCYQQIS |
| | | PRIYDPSDPKGVILPQIYAGYDRKPEDIISAGVPNRLAIPEGQPG |
| | | YIPEHQRAGLKTQGRIRCRASVEAKARAAILAVVHLGEDWV |
| | | VLDLRGLLRNVYWRKLASPGTLTLKGLLDFFTGGPVLDARR |
| | | GIATFSYTLKSAAAVHAENTYKGKGTREVLLKLTENNSVALV |
| | | TVDLGQRNPLAAMIARVSRTSQGDLTYPESVEPLTRLFLPDPF |
| | | LEEVRKYRSSYDALRLSIREAAIASLTPEQQAEIRYIEKFSAGD |
| | | AKKNVAEVFGIDPTQLPWDAMTPRTTYISDLFLRMGGDRSRV |
| | | FFEVPPKKAKKAPKKPPKKPAGPRIVKRTDGMIARLREIRPRL |
| | | SAETNKAFQEARWEGERSNVAFQKLSVRRKQFARTVVNHLV |
| | | QTAQKMSRCDTVVLGIEDLNVPFFHGRGKYQPGWEGFFRQK |
| | | KENRWLINDMHKALSERGPHRGGYVLELTPFWTSLRCPKCG |
| | | HTDSANRDGDDFVCVKCGAKLHSDLEVATANLALVAITGQSI |
| | | PRPPREQSSGKKSTGTARMKKTSGETQGKGSKACVSEALNKI |
| | | EQGTARDPVYNPLNSQVSCPAP |
| CasΦ.24 | SEQ ID | VYNPDMKKPNNIRRIREEHFEGLCFGKDVLTKAGKIYEKDGE |
| | NO: 244 | EAAIDFLMGKDEEDPPNFKPPAKTTIVAQSRPFDQWPIYQVSQ |
| | | AVQERVFAYTEEEFNASKEALFSGDISSKSRDFWFKTNNISDQ |
| | | GIGAQGLNTILSHAFSRYSGVIKKVENRNKKRLKKLSKKNQL |
| | | KIEEGLEILEFKPDSAFNENGLLAQPPGINPNIYGYQAVTPFVF |
| | | DPDNPGDVILPKQYEGYSRKPDDIIEKGPSRLDIPKGQPGYVPE |
| | | HQRKNLKKKGRVRLYRRTPPKTKALASILAVLQIGKDWVLF |
| | | DMRGLLRSVYMREAATPGQISAKDLLDTFTGCPVLNTRTGEF |
| | | TFCYKLRSEGALHARKIYTKGETRTLLTSLTSENNTIALVTVD |
| | | LGQRNPAAIMISRLSRKEELSEKDIQPVSRRLLPDRYLNELKR |
| | | YRDAYDAFRQEVRDEAFTSLCPEHQEQVQQYEALTPEKAKN |
| | | LVLKHFFGTHDPDLPWDDMTSNTHYIANLYLERGGDPSKVFF |
| | | TRPLKKDSKSKKPRKPTKRTDASISRLPEIRPKMPEDARKAFE |
| | | KAKWEIYTGHEKFPKLAKRVNQLCREIANWIEKEAKRLTLCD |
| | | TVVVGIEDLSLPPKRGKGKFQETWQGFFRQKFENRWVIDTLK |
| | | KAIQNRAHDKGKYVLGLAPYWTSQRCPACGFIHKSNRNGDH |
| | | FKCLKCEALFHADSEVATWNLALVAVLGKGITNPDSKKPSGQ |
| | | KKTGTTRKKQIKGKNKGKETVNVPPTTQEVEDIIAFFEKDDET |
| | | VRNPVYKPTGT |
| CasΦ.25 | SEQ ID | MKKPNNIRRIREEHFEGLCFGKDVLTKAGKIYEKDGEEAAIDF |
| | NO: 245 | LMGKDEEDPPNFKPPAKTTIVAQSRPFDQWPIYQVSQAVQER |
| | | VFAYTEEEFNASKEALFSGDISSKSRDFWFKTNNISDQGIGAQ |
| | | GLNTILSHAFSRYSGVIKKVENRNKKRLKKLSKKNQLKIEEGL |
| | | EILEFKPDSAFNENGLLAQPPGINPNIYGYQAVTPFVFDPDNPG |
| | | DVILPKQYEGYSRKPDDIIEKGPSRLDIPKGQPGYVPEHQRKN |
| | | LKKKGRVRLYRRTPPKTKALASILAVLQIGKDWVLFDMRGLL |
| | | RSVYMREAATPGQISAKDLLDTFTGCPVLNTRTGEFTFCYKL |
| | | RSEGALHARKIYTKGETRTLLTSLTSENNTIALVTVDLGQRNP |
| | | AAIMISRLSRKEELSEKDIQPVSRRLLPDRYLNELKRYRDAYD |
| | | AFRQEVRDEAFTSLCPEHQEQVQQYEALTPEKAKNLVLKHFF |
| | | GTHDPDLPWDDMTSNTHYIANLYLERGGDPSKVFFTRPLKKD |
| | | SKSKKPRKPTKRTDASISRLPEIRPKMPEDARKAFEKAKWEIY |
| | | TGHEKFPKLAKRVNQLCREIANWIEKEAKRLTLCDTVVVGIE |
| | | DLSLPPKRGKGKFQETWQGFFRQKFENRWVIDTLKKAIQNRA |
| | | HDKGKYVLGLAPYWTSQRCPACGFIHKSNRNGDHFKCLKCE |
| | | ALFHADSEVATWNLALVAVLGKGITNPDSKKPSGQKKTGTT |
| | | RKKQIKGKNKGKETVNVPPTTQEVEDIIAFFEKDDETVRNPV |
| | | YKPTGT |
| CasΦ.26 | SEQ ID | VIKTHFPAGRFRKDHQKTAGKKLKHEGEEACVEYLRNKVSD |
| | NO: 246 | YPPNFKPPAKGTIVAQSRPFSEWPIVRASEAIQKYVYGLTVAE |
| | | LDVFSPGTSKPSHAEWFAKTGVENYGYRQVQGLNTIFQNTVN |
| | | RFKGVLKKVENRNKKSLKRQEGANRRRVEEGLPEVPVTVES |
| | | ATDDEGRLLQPPGVNPSIYGYQGVAPRVCTDLQGFSGMSVDF |
| | | AGYRRDPDAVLVESLPEGRLSIPKGERGYVPEWQRDPERNKF |
| | | PLREGSRRQRKWYSNACHKPKPGRTSKYDPEALKKASAKDA |
| | | LLVSISIGEDWAIIDVRGLLRDARRRGFTPEEGLSLNSLLGLFT |
| | | EYPVFDVQRGLITFTYKLGQVDVHSRKTVPTFRSRALLESLVA |
| | | KEEIALVSVDLGQTNPASMKVSRVRAQEGALVAEPVHRMFL |
| | | SDVLLGELSSYRKRMDAFEDAIRAQAFETMTPEQQAEITRVC |
| | | DVSVEVARRRVCEKYSISPQDVPWGEMTGHSTFIVDAVLRKG |
| | | GDESLVYFKNKEGETLKFRDLRISRMEGVRPRLTKDTRDALN |
| | | KAVLDLKRAHPTFAKLAKQKLELARRCVNFIEREAKRYTQCE |
| | | RVVFVIEDLNVGFFHGKGKRDRGWDAFFTAKKENRWVIQAL |
| | | HKAFSDLGLHRGSYVIEVTPQRTSMTCPRCGHCDKGNRNGE |
| | | KFVCLQCGATLHADLEVATDNIERVALTGKAMPKPPVRERSG |
| | | DVQKAGTARKARKPLKPKQKTEPSVQEGSSDDGVDKSPGDA |
| | | SRNPVYNPSDTLSI |
| CasΦ.27 | SEQ ID | MAKAKTLAALLRELLPGQHLAPHHRWVANKLLMTSGDAAA |
| | NO: 247 | FVIGKSVSDPVRGSFRKDVITKAGRIFKKDGPDAAAAFLDGK |
| | | WEDRPPNFQPPAKAAIVAISRSFDEWPIVKVSCAIQQYLYALP |
| | | VQEFESSVPEARAQAHAAWFQDTGVDDCNFKSTQGLNAIFN |
| | | HGKRTYEGVLKKAQNRNDKKNLRLERINAKRAEAGQAPLVA |
| | | GPDESPTDDAGCLLHPPGINANIYCYQQVSPRPYEQSCGIQLPP |
| | | EYAGYNRLSNVAIPPMPNRLDIPQGQPGYVPEHHRHGIKKFG |
| | | RVRKRYGVVPGRNRDADGKRTRQVLTEAGAAAKARDSVLA |
| | | VIRIGDDWTVVDLRGLLRNAQWRKLVPDGGITVQGLLDLFT |
| | | GDPVIDPRRGVVTFIYKADSVGIHSEKVCRGKQSKNLLERLCA |
| | | MPEKSSTRLDCARQAVALVSVDLGQRNPVAARFSRVSLAEG |
| | | QLQAQLVSAQFLDDAMVAMIRSYREEYDRFESLVREQAKAA |
| | | LSPEQLSEIVRHEADSAESVKSCVCAKFGIDPAGLSWDKMTS |
| | | GTWRIADHVQAAGGDVEWFFFKTCGKGKEIKTVRRSDFNVA |
| | | KQFRLRLSPETRKDWNDAIWELKRGNPAYVSFSKRKSEFARR |
| | | VVNDLVHRARRAVRCDEVVFAIEDLNISFFHGKGQRQMGWD |
| | | AFFEVKQENRWFIQALHKAFVERATHKGGYVLEVAPARTSTT |
| | | CPECRHCDPESRRGEQFCCIKCRHTCHADLEVATFNIEQVALT |
| | | GVSLPKRLSSTLL |
| CasΦ.28 | SEQ ID | MSKEKTPPSAYAILKAKHFPDLDFEKKHKMMAGRMFKNGAS |
| | NO: 248 | EQEVVQYLQGKGSESLMDVKPPAKSPILAQSRPFDEWEMVRT |
| | | SRLIQETIFGIPKRGSIPKRDGLSETQFNELVASLEVGGKPMLN |
| | | KQTRAIFYGLLGIKPPTFHAMAQNILIDLAINIRKGVLKKVDN |
| | | LNEKNRKKVKRIRDAGEQDVMVPAEVTAHDDRGYLNHPPG |
| | | VNPTIPGYQGVVIPFPEGFEGLPSGMTPVDWSHVLVDYLPHD |
| | | RLSIPKGSPGYIPEWQRPLLNRHKGRRHRSWYANSLNKPRKS |
| | | RTEEAKDRQNAGKRTALIEAERLKGVLPVLMRFKEDWLIIDA |
| | | RGLLRNARYRGVLPEGSTLGNLIDLFSDSPRVDTRRGICTFLY |
| | | RKGRAYSTKPVKRKESKETLLKLTEKSTIALVSIDLGQTNPLT |
| | | AKLSKVRQVDGCLVAEPVLRKLIDNASEDGKEIARYRVAHDL |
| | | LRARILEDAIDLLGIYKDEVVRARSDTPDLCKERVCRFLGLDS |
| | | QAIDWDRMTPYTDFIAQAFVAKGGDPKVVTIKPNGKPKMFR |
| | | KDRSIKNMKGIRLDISKEASSAYREAQWAIQRESPDFQRLAV |
| | | WQSQLTKRIVNQLVAWAKKCTQCDTVVLAFEDLNIGMMHG |
| | | SGKWANGGWNALFLHKQENRWFMQAFHKALTELSAHKGIP |
| | | TIEVLPHRTSITCTQCGHCHPGNRDGERFKCLKCEFLANTDLEI |
| | | ATDNIERVALTGLPMPKGERSSAKRKPGGTRKTKKSKHSGNS |
| | | PLAAE |
| CasΦ.29 | SEQ ID | MEKAGPTSPLSVLIHKNFEGCRFQIDHLKIAGRKLAREGEAAA |
| | NO: 249 | IEYLLDKKCEGLPPNFQPPAKGNVIAQSRPFTEWAPYRASVAI |
| | | QKYIYSLSVDERKVCDPGSSSDSHEKWFKQTGVQNYGYTHV |
| | | QGLNLIFKHALARYDGVLKKVDNRNEKNRKKAERVNSFRRE |
| | | EGLPEEVFEEEKATDETGHLLQPPGVNHSIYCYQSVRPKPFNP |
| | | RKPGGISLPEAYSGYSLKPQDELPIGSLDRLSIPPGQPGYVPEW |
| | | QRSQLTTQKHRRKRSWYSAQKWKPRTGRTSTFDPDRLNCAR |
| | | AQGAILAVVRIHEDWVVFDVRGLLRNALWRELAGKGLTVRD |
| | | LLDFFTGDPVVDTKRGVVTFTYKLGKVDVHSLRTVRGKRSK |
| | | KVLEDLTLSSDVGLVTIDLGQTNVLAADYSKVTRSENGELLA |
| | | VPLSKSFLPKHLLHEVTAYRTSYDQMEEGFRRKALLTLTEDQ |
| | | QVEVTLVRDFSVESSKTKLLQLGVDVTSLPWEKMSSNTTYIS |
| | | DQLLQQGADPASLFFDGERDGKPCRHKKKDRTWAYLVRPKV |
| | | SPETRKALNEALWALKNTSPEFESLSKRKIQFSRRCMNYLLNE |
| | | AKRISGCGQVVFVIEDLNVRVHHGRGKRAIGWDNFFKPKREN |
| | | RWFMQALHKAASELAIHRGMHIIEACPARSSITCPKCGHCDPE |
| | | NRCSSDREKFLCVKCGAAFHADLEVATFNLRKVALTGTALPK |
| | | SIDHSRDGLIPKGARNRKLKEPQANDEKACA |
| CasΦ.30 | SEQ ID | MKEQSPLSSVLKSNFPGKKFLSADIRVAGRKLAQLGEAAAVE |
| | NO: 250 | YLSPRQRDSVPNFRPPAFCTVVAKSRPFEEWPIYKASVLLQEQ |
| | | TYGMTGQEFEERCGSIPTSLSGLRQWASSVGLGAAMEGLHVQ |
| | | GMNLMVKNAINRYKGVLVKVENRNKKLVEANEAKNSSREE |
| | | RGLPPLRPPELGSAFGPDGRLVNPPGIDKSIRLYQGVSPVPVV |
| | | KTTGRPTVHRLDIPAGEKGHVPLWQREAGLVKEGPRRRRMW |
| | | YSNSNLKRSRKDRSAEASEARKADSVVVRVSVKEDWVDIDV |
| | | RGLLRNVAWRGIERAGESTEDLLSLFSGDPVVDPSRDSVVFL |
| | | YKEGVVDVLSKKVVGAGKSRKQLEKMVSEGPVALVSCDLG |
| | | QTNYVAARVSVLDESLSPVRSFRVDPREFPSADGSQGVVGSL |
| | | DRIRADSDRLEAKLLSEAEASLPEPVRAEIEFLRSERPSAVAGR |
| | | LCLKLGIDPRSIPWEKMGSTTSFISEALSAKGSPLALHDGAPIK |
| | | DSRFAHAARGRLSPESRKALNEALWERKSSSREYGVISRRKSE |
| | | ASRRMANAVLSESRRLTGLAVVAVNLEDLNMVSKFFHGRGK |
| | | RAPGWAGFFTPKMENRWFIRSIHKAMCDLSKHRGITVIESRPE |
| | | RTSISCPECGHCDPENRSGERFSCKSCGVSLHADFEVATRNLE |
| | | RVALTGKPMPRRENLHSPEGATASRKTRKKPREATASTFLDL |
| | | RSVLSSAENEGSGPAARAG |
| CasΦ.31 | SEQ ID | MLPPSNKIGKSMSLKEFINKRNFKSSIIKQAGKILKKEGEEAVK |
| | NO: 251 | KYLDDNYVEGYKKRDFPITAKCNIVASNRKIEDFDISKFSSFIQ |
| | | NYVFNLNKDNFEEFSKIKYNRKSFDELYKKIANEIGLEKPNYE |
| | | NIQGEIAVIRNAINIYNGVLKKVENRNKKIQEKNQSKDPPKLL |
| | | SAFDDNGFLAERPGINETIYGYQSVRLRHLDVEKDKDIIVQLP |
| | | DIYQKYNKKSTDKISVKKRLNKYNVDEYGKLISKRRKERINK |
| | | DDAILCVSNFGDDWIIFDARGLLRQTYRYKLKKKGLCIKDLL |
| | | NLFTGDPIINPTKTDLKEALSLSFKDGIINNRTLKVKNYKKCPE |
| | | LISELIRDKGKVAMISIDLGQTNPISYRLSKFTANNVAYIENGV |
| | | ISEDDIVKMKKWREKSDKLENLIKEEAIASLSDDEQREVRLYE |
| | | NDIADNTKKKILEKFNIREEDLDFSKMSNNTYFIRDCLKNKNI |
| | | DESEFTFEKNGKKLDPTDACFAREYKNKLSELTRKKINEKIWE |
| | | IKKNSKEYHKISIYKKETIRYIVNKLIKQSKEKSECDDIIVNIEK |
| | | LQIGGNFFGGRGKRDPGWNNFFLPKEENRWFINACHKAFSEL |
| | | APHKGIIVIESDPAYTSQTCPKCENCDKENRNGEKFKCKKCNY |
| | | EANADIDVATENLEKIAKNGRRLIKNFDQLGERLPGAEMPGG |
| | | ARKRKPSKSLPKNGRGAGVGSEPELINQSPSQVIA |
| CasΦ.32 | SEQ ID | VPDKKETPLVALCKKSFPGLRFKKHDSRQAGRILKSKGEGAA |
| | NO: 252 | VAFLEGKGGTTQPNFKPPVKCNIVAMSRPLEEWPIYKASVVIQ |
| | | KYVYAQSYEEFKATDPGKSEAGLRAWLKATRVDTDGYFNV |
| | | QGLNLIFQNARATYEGVLKKVENRNSKKVAKIEQRNEHRAE |
| | | RGLPLLTLDEPETALDETGHLRHRPGINCSVFGYQHMKLKPY |
| | | VPGSIPGVTGYSRDPSTPIAACGVDRLEIPEGQPGYVPPWDRE |
| | | NLSVKKHRRKRASWARSRGGAIDDNMLLAVVRVADDWALL |
| | | DLRGLLRNTQYRKLLDRSVPVTIESLLNLVTNDPTLSVVKKPG |
| | | KPVRYTATLIYKQGVVPVVKAKVVKGSYVSKMLDDTTETFS |
| | | LVGVDLGVNNLIAANALRIRPGKCVERLQAFTLPEQTVEDFF |
| | | RFRKAYDKHQENLRLAAVRSLTAEQQAEVLALDTFGPEQAK |
| | | MQVCGHLGLSVDEVPWDKVNSRSSILSDLAKERGVDDTLYM |
| | | FPFFKGKGKKRKTEIRKRWDVNWAQHFRPQLTSETRKALNE |
| | | AKWEAERNSSKYHQLSIRKKELSRHCVNYVIRTAEKRAQCG |
| | | KVIVAVEDLHHSFRRGGKGSRKSGWGGFFAAKQEGRWLMD |
| | | ALFGAFCDLAVHRGYRVIKVDPYNTSRTCPECGHCDKANRD |
| | | RVNREAFICVCCGYRGNADIDVAAYNIAMVAITGVSLRKAAR |
| | | ASVASTPLESLAAE |
| CasΦ.33 | SEQ ID | MSKTKELNDYQEALARRLPGVRHQKSVRRAARLVYDRQGE |
| | NO: 253 | DAMVAFLDGKEVDEPYTLQPPAKCHILAVSRPIEEWPIARVT |
| | | MAVQEHVYALPVHEVEKSRPETTEGSRSAWFKNSGVSNHGV |
| | | THAQTLNAILKNAYNVYNGVIKKVENRNAKKRDSLAAKNKS |
| | | RERKGLPHFKADPPELATDEQGYLLQPPSPNSSVYLVQQHLR |
| | | TPQIDLPSGYTGPVVDPRSPIPSLIPIDRLAIPPGQPGYVPLHDR |
| | | EKLTSNKHRRMKLPKSLRAQGALPVCFRVFDDWAVVDGRGL |
| | | LRHAQYRRLAPKNVSIAELLELYTGDPVIDIKRNLMTFRFAEA |
| | | VVEVTARKIVEKYHNKYLLKLTEPKGKPVREIGLVSIDLNVQ |
| | | RLIALAIYRVHQTGESQLALSPCLHREILPAKGLGDFDKYKSK |
| | | FNQLTEEILTAAVQTLTSAQQEEYQRYVEESSHEAKADLCLK |
| | | YSITPHELAWDKMTSSTQYISRWLRDHGWNASDFTQITKGRK |
| | | KVERLWSDSRWAQELKPKLSNETRRKLEDAKHDLQRANPE |
| | | WQRLAKRKQEYSRHLANTVLSMAREYTACETVVIAIENLPM |
| | | KGGFVDGNGSRESGWDNFFTHKKENRWMIKDIHKALSDLAP |
| | | NRGVHVLEVNPQYTSQTCPECGHRDKANRDPIQRERFCCTHC |
| | | GAQRHADLEVATHNIAMVATTGKSLTGKSLAPQRLQEAAE |
| CasΦ.41 | SEQ ID | VLLSDRIQYTDPSAPIPAMTVVDRRKIKKGEPGYVPPFMRKNL |
| | NO: 254 | STNKHRRMRLSRGQKEACALPVGLRLPDGKDGWDFIIFDGRA |
| | | LLRACRRLRLEVTSMDDVLDKFTGDPRIQLSPAGETIVTCML |
| | | KPQHTGVIQQKLITGKMKDRLVQLTAEAPIAMLTVDLGEHNL |
| | | VACGAYTVGQRRGKLQSERLEAFLLPEKVLADFEGYRRDSD |
| | | EHSETLRHEALKALSKRQQREVLDMLRTGADQARESLCYKY |
| | | GLDLQALPWDKMSSNSTFIAQHLMSLGFGESATHVRYRPKR |
| | | KASERTILKYDSRFAAEEKIKLTDETRRAWNEAIWECQRASQ |
| | | EFRCLSVRKLQLARAAVNWTLTQAKQRSRCPRVVVVVEDLN |
| | | VRFMHGGGKRQEGWAGFFKARSEKRWFIQALHKAYTELPTN |
| | | RGIHVMEVNPARTSITCTKCGYCDPENRYGEDFHCRNPKCKV |
| | | RGGHVANADLDIATENLARVALSGPMPKAPKLK |
| CasΦ.34 | SEQ ID | MTPSFGYQMIIVTPIHHASGAWATLRLLFLNPKTSGVMLGMT |
| | NO: 255 | KTKSAFALMREEVFPGLLFKSADLKMAGRKFAKEGREAAIEY |
| | | LRGKDEERPANFKPPAKGDIIAQSRPFDQWPIVQVSQAIQKYIF |
| | | GLTKAEFDATKTLLYGEGNHPTTESRRRWFEATGVPDFGFTS |
| | | AQGLNAIFSSALARYEGVIQKVENRNEKRLKKLSEKNQRLVE |
| | | EGHAVEAYVPETAFHTLESLKALSEKSLVPLDDLMDKIDRLA |
| | | QPPGINPCLYGYQQVAPYIYDPENPRGVVLPDLYLGYCRKPD |
| | | DPITACPNRLDIPKGQPGYIPEHQRGQLKKHGRVRRFRYTNPQ |
| | | AKARAKAQTAILAVLRIDEDWVVMDLRGLLRNVYFREVAAP |
| | | GELTARTLLDTFTGCPVLNLRSNVVTFCYDIESKGALHAEYV |
| | | RKGWATRNKLLDLTKDGQSVALLSVDLGQRHPVAVMISRLK |
| | | RDDKGDLSEKSIQVVSRTFADQYVDKLKRYRVQYDALRKEI |
| | | YDAALVSLPPEQQAEIRAYEAFAPGDAKANVLSVMFQGEVSP |
| | | DELPWDKMNTNTHYISDLYLRRGGDPSRVFFVPQPSTPKKNA |
| | | KKPPAPRKPVKRTDENVSHMPEFRPHLSNETREAFQKAKWT |
| | | MERGNVRYAQLSRFLNQIVREANNWLVSEAKKLTQCQTVV |
| | | WAIEDLHVPFFHGKGKYHETWDGFFRQKKEDRWFVNVFHK |
| | | AISERAPNKGEYVMEVAPYRTSQRCPVCGFVDADNRHGDHF |
| | | KCLRCGVELHADLEVATWNIALVAVQGHGIAGPPREQSCGG |
| | | ETAGTARKGKNIKKNKGLADAVTVEAQDSEGGSKKDAGTAR |
| | | NPVYIPSESQVNCPAP |
| CasΦ.35 | SEQ ID | MKPKTPKPPKTPVAALIDKHFPGKRFRASYLKSVGKKLKNQG |
| | NO: 256 | EDVAVRFLTGKDEERPPNFQPPAKSNIVAQSRPIEEWPIHKVS |
| | | VAVQEYVYGLTVAEKEACSDAGESSSSHAAWFAKTGVENFG |
| | | YTSVQGLNKIFPPTFNRFDGVIKKVENRNEKKRQKATRINEAK |
| | | RNKGQSEDPPEAEVKATDDAGYLLOPPGINHSVYGYQSITLC |
| | | PYTAEKFPTIKLPEEYAGYHSNPDAPIPAGVPDRLAIPEGQPGH |
| | | VPEEHRAGLSTKKHRRVRQWYAMANWKPKPKRTSKPDYDR |
| | | LAKARAQGALLIVIRIDEDWVVVDARGLLRNVRWRSLGKREI |
| | | TPNELLDLFTGDPVLDLKRGVVTFTYAEGVVNVCSRSTTKGK |
| | | QTKVLLDAMTAPRDGKKRQIGMVAVDLGQTNPIAAEYSRVG |
| | | KNAAGTLEATPLSRSTLPDELLREIALYRKAHDRLEAQLREEA |
| | | VLKLTAEQQAENARYVETSEEGAKLALANLGVDTSTLPWDA |
| | | MTGWSTCISDHLINHGGDTSAVFFQTIRKGTKKLETIKRKDSS |
| | | WADIVRPRLTKETREALNDFLWELKRSHEGYEKLSKRLEELA |
| | | RRAVNHVVQEVKWLTQCQDIVIVIEDLNVRNFHGGGKRGGG |
| | | WSNFFTVKKENRWFMQALHKAFSDLAAHRGIPVLEVYPART |
| | | SITCLGCGHCDPENRDGEAFVCQQCGATFHADLEVATRNIAR |
| | | VALTGEAMPKAPAREQPGGAKKRGTSRRRKLTEVAVKSAEP |
| | | TIHQAKNQQLNGTSRDPVYKGSELPAL |
| CasΦ.43 | SEQ ID | MSEITDLLKANFKGKTFKSADMRMAGRILKKSGAQAVIKYLS |
| | NO: 257 | DKGAVDPPDFRPPAKCNIIAQSRPFDEWPICKASMAIQQHIYG |
| | | LTKNEFDESSPGTSSASHEQWFAKTGVDTHGFTHVQGLNLIF |
| | | QHAKKRYEGVIKKVENYNEKERKKFEGINERRSKEGMPLLEP |
| | | RLRTAFGDDGKFAEKPGVNPSIYLYQQTSPRPYDKTKHPYVH |
| | | APFELKEITTIPTQDDRLKIPFGAPGHVPEKHRSQLSMAKHKR |
| | | RRAWYALSQNKPRPPKDGSKGRRSVRDLADLKAASLADAIP |
| | | LVSRVGFDWVVIDGRGLLRNLRWRKLAHEGMTVEEMLGFFS |
| | | GDPVIDPRRNVATFIYKAEHATVKSRKPIGGAKRAREELLKAT |
| | | ASSDGVIRQVGLISVDLGQTNPVAYEISRMHQANGELVAEHL |
| | | EYGLLNDEQVNSIQRYRAAWDSMNESFRQKAIESLSMEAQD |
| | | EIMQASTGAAKRTREAVLTMFGPNATLPWSRMSSNTTCISDA |
| | | LIEVGKEEETNFVTSNGPRKRTDAQWAAYLRPRVNPETRALL |
| | | NQAVWDLMKRSDEYERLSKRKLEMARQCVNFVVARAEKLT |
| | | QCNNIGIVLENLVVRNFHGSGRRESGWEGFFEPKRENRWFMQ |
| | | VLHKAFSDLAQHRGVMVFEVHPAYSSQTCPACRYVDPKNRS |
| | | SEDRERFKCLKCGRSFNADREVATFNIREIARTGVGLPKPDCE |
| | | RSRGVQTTGTARNPGRSLKSNKNPSEPKRVLQSKTRKKITSTE |
| | | TQNEPLATDLKT |
| CasΦ.44 | SEQ ID | MTPKTESPLSALCKKHFPGKRFRTNYLKDAGKILKKHGEDAV |
| | NO: 258 | VAFLSDKQEDEPANFCPPAKVHILAQSRPFEDWPINLASKAIQ |
| | | TYVYGLTADERKTCEPGTSKESHDRWFKETGVDHHGFTSVQ |
| | | GLNLIFKHTLNRYDGVIKKVETRNEKRRSSVVRINEKKAAEG |
| | | LPLIAAEAEETAFGEDGRLLQPPGVNHSIYCFQQVSPQPYSSK |
| | | KHPQVVLPHAVQGVDPDAPIPVGRPNRLDIPKGQPGYVPEWQ |
| | | RPHLSMKCKRVRMWYARANWRRKPGRRSVLNEARLKEASA |
| | | KGALPIVLVIGDDWLVMDARGLLRSVFWRRVAKPGLSLSELL |
| | | NVTPTGLFSGDPVIDPKRGLVTFTSKLGVVAVHSRKPTRGKK |
| | | SKDLLLKMTKPTDDGMPRHVGMVAIDLGQTNPVAAEYSRVV |
| | | QSDAGTLKQEPVSRGVLPDDLLKDVARYRRAYDLTEESIRQE |
| | | AIALLSEGHRAEVTKLDQTTANETKRLLVDRGVSESLPWEKM |
| | | SSNTTYISDCLVALGKTDDVFFVPKAKKGKKETGIAVKRKDH |
| | | GWSKLLRPRTSPEARKALNENQWAVKRASPEYERLSRRKLEL |
| | | GRRCVNHIIQETKRWTQCEDIVVVLEDLNVGFFHGSGKRPDG |
| | | WDNFFVSKRENRWFIQVLHKAFGDLATHRGTHVIEVHPARTS |
| | | ITCIKCGHCDAGNRDGESFVCLASACGDRRHADLEVATRNVA |
| | | RVAITGERMPPSEQARDVQKAGGARKRKPSARNVKSSYPAV |
| | | EPAPASP |
| CasΦ.36 | SEQ ID | MSDNKMKKLSKEEKPLTPLQILIRKYIDKSQYPSGFKTTIIKQA |
| | NO: 259 | GVRIKSVKSEQDEINLANWIISKYDPTYIKRDFNPSAKCQIIAT |
| | | SRSVADFDIVKMSNKVQEIFFASSHLDKNVFDIGKSKSDHDS |
| | | WFERNNVDRGIYTYSNVQGMNLIFSNTKNTYLGVAVKAQNK |
| | | FSSKMKRIQDINNFRITNHQSPLPIPDEIKIYDDAGFLLNPPGVN |
| | | PNIFGYQSCLLKPLENKEIISKTSFPEYSRLPADMIEVNYKISNR |
| | | LKFSNDQKGFIQFKDKLNLFKINSQELFSKRRRLSGQPILLVAS |
| | | FGDDWVVLDGRGLLRQVYYRGIAKPGSITISELLGFFTGDPIV |
| | | DPIRGVVSLGFKPGVLSQETLKTTSARIFAEKLPNLVLNNNVG |
| | | LMSIDLGQTNPVSYRLSEITSNMSVEHICSDFLSQDQISSIEKA |
| | | KTSLDNLEEEIAIKAVDHLSDEDKINFANFSKLNLPEDTRQSLF |
| | | EKYPELIGSKLDFGSMGSGTSYIADELIKFENKDAFYPSGKKK |
| | | FDLSFSRDLRKKLSDETRKSYNDALFLEKRTNDKYLKNAKRR |
| | | KQIVRTVANSLVSKIEELGLTPVINIENLAMSGGFFDGRGKRE |
| | | KGWDNFFKVKKENRWVMKDFHKAFSELSPHHGVIVIESPPY |
| | | CTSVTCTKCNFCDKKNRNGHKFTCQRCGLDANADLDIATEN |
| | | LEKVAISGKRMPGSERSSDERKVAVARKAKSPKGKAIKGVKC |
| | | TITDEPALLSANSQDCSQSTS |
| CasΦ.37 | SEQ ID | MALSLAEVRERHFKGLRFRSSYLKRAGKILKKEGEAACVAYL |
| | NO: 260 | TGKDEESPPNFKPPAKCDVVAQSRPFEEWPIVQASVAVQSYV |
| | | YGLTKEAFEAFNPGTTKQSHEACLAATGIDTCGYSNVQGLNL |
| | | IFRQAKNRYEGVITKVENRNKKAKKKLTRKNEWRQKNGHSE |
| | | LPEAPEELTFNDEGRLLQPPGINPSLYTYQQISPTPWSPKDSSIL |
| | | PPQYAGYERDPNAPIPFGVAKDRLTIASGCPGYIPEWMRTAGE |
| | | KTNPRTQKKFMHPGLSTRKNKRMRLPRSVRSAPLGALLVTIH |
| | | LGEDWLVLDVRGLLRNARWRGVAPKDISTQGLLNLFTGDPVI |
| | | DTRRGVVTFTYKPETVGIHSRTWLYKGKQTKEVLEKLTQDQ |
| | | TVALVAIDLGQTNPVSAAASRVSRSGENLSIETVDRFFLPDELI |
| | | KELRLYRMAHDRLEERIREESTLALTEAQQAEVRALEHVVRD |
| | | DAKNKVCAAFNLDAASLPWDQMTSNTTYLSEAILAQGVSRD |
| | | QVFFTPNPKKGSKEPVEVMRKDRAWVYAFKAKLSEETRKAK |
| | | NEALWALKRASPDYARLSKRREELCRRSVNMVINRAKKRTQ |
| | | CQVVIPVLEDLNIGFFHGSGKRLPGWDNFFVAKKENRWLMN |
| | | GLHKSFSDLAVHRGFYVFEVMPHRTSITCPACGHCDSENRDG |
| | | EAFVCLSCKRTYHADLDVATHNLTQVAGTGLPMPEREHPGG |
| | | TKKPGGSRKPESPQTHAPILHRTDYSESADRLGS |
| CasΦ.45 | SEQ ID | QAVIKYLSDKGAVDPPDFRPPAKCNIIAQSRPFDEWPICKASM |
| | NO: 261 | AIQQHIYGLTKNEFDESSPGTSSASHEQWFAKTGVDTHGFTH |
| | | VQGLNLIFQHAKKRYEGVIKKVENYNEKERKKFEGINERRSK |
| | | EGMPLLEPRLRTAFGDDGKFAEKPGVNPSIYLYQQTSPRPYD |
| | | KTKHPYVHAPFELKEITTIPTQDDRLKIPFGAPGHVPEKHRSQL |
| | | SMAKHKRRRAWYALSQNKPRPPKDGSKGRRSVRDLADLKA |
| | | ASLADAIPLVSRVGFDWVVIDGRGLLRNLRWRKLAHEGMTV |
| | | EEMLGFFSGDPVIDPRRNVATFIYKAEHATVKSRKPIGGAKRA |
| | | REELLKATASSDGVIRQVGLISVDLGQTNPVAYEISRMHQAN |
| | | GELVAEHLEYGLLNDEQVNSIQRYRAAWDSMNESFRQKAIES |
| | | LSMEAQDEIMQASTGAAKRTREAVLTMFGPNATLPWSRMSS |
| | | NTTCISDALIEVGKEEETNFVTSNGPRKRTDAQWAAYLRPRV |
| | | NPETRALLNQAVWDLMKRSDEYERLSKRKLEMARQCVNFV |
| | | VARAEKLTQCNNIGIVLENLVVRNFHGSGRRESGWEGFFEPK |
| | | RENRWFMQVLHKAFSDLAQHRGVMVFEVHPAYSSQTCPAC |
| | | RYVDPKNRSSEDRERFKCLKCGRSFNADREVATFNIREIARTG |
| | | VGLPKPDCERSRDVQTPGTARKSGRSLKSQDNLSEPKRVLQS |
| | | KTRKKITSTETQNEPLATDLKT |
| CasΦ.38 | SEQ ID | MIKEQSELSKLIEKYYPGKKFYSNDLKQAGKHLKKSEHLTAK |
| | NO: 262 | ESEELTVEFLKSCKEKLYDFRPPAKALIISTSRPFEEWPIYKASE |
| | | SIQKYIYSLTKEELEKYNISTDKTSQENFFKESLIDNYGFANVS |
| | | GLNLIFQHTKAIYDGVLKKVNNRNNKILKKYKRKIEEGIEIDS |
| | | PELEKAIDESGHFINPPGINKNIYCYQQVSPTIFNSFKETKIICPF |
| | | NYKRNPNDIIQKGVIDRLAIPFGEPGYIPDHQRDKVNKHKKRI |
| | | RKYYKNNENKNKDAILAKINIGEDWVLFDLRGLLRNAYWRK |
| | | LIPKQGITPQQLLDMFSGDPVIDPIKNNITFIYKESIIPIHSESIIKT |
| | | KKSKELLEKLTKDEQIALVSIDLGQTNPVAARFSRLSSDLKPE |
| | | HVSSSFLPDELKNEICRYREKSDLLEIEIKNKAIKMLSQEQQDE |
| | | IKLVNDISSEELKNSVCKKYNIDNSKIPWDKMNGFTTFIADEFI |
| | | NNGGDKSLVYFTAKDKKSKKEKLVKLSDKKIANSFKPKISKE |
| | | TREILNKITWDEKISSNEYKKLSKRKLEFARRATNYLINQAKK |
| | | ATRLNNVVLVVEDLNSKFFHGSGKREDGWDNFFIPKKENRW |
| | | FIQALHKSLTDVSIHRGINVIEVRPERTSITCPKCGCCDKENRK |
| | | GEDFKCIKCDSVYHADLEVATFNIEKVAITGESMPKPDCERLG |
| | | GEESIG |
| CasΦ.39 | SEQ ID | VAFLDGKEVDEPYTLQPPAKCHILAVSRPIEEWPIARVTMAV |
| | NO: 263 | QEHVYALPVHEVEKSRPETTEGSRSAWFKNSGVSNHGVTHA |
| | | QTLNAILKNAYNVYNGVIKKVENRNAKKRDSLAAKNKSRER |
| | | KGLPHFKADPPELATDEQGYLLQPPSPNSSVYLVQQHLRTPQI |
| | | DLPSGYTGPVVDPRSPIPSLIPIDRLAIPPGQPGYVPLHDREKLT |
| | | SNKHRRMKLPKSLRAQGALPVCFRVFDDWAVVDGRGLLRH |
| | | AQYRRLAPKNVSIAELLELYTGDPVIDIKRNLMTFRFAEAVVE |
| | | VTARKIVEKYHNKYLLKLTEPKGKPVREIGLVSIDLNVQRLIA |
| | | LAIYRVHQTGESQLALSPCLHREILPAKGLGDFDKYKSKFNQL |
| | | TEEILTAAVQTLTSAQQEEYQRYVEESSHEAKADLCLKYSITP |
| | | HELAWDKMTSSTQYISRWLRDHGWNASDFTQITKGRKKVER |
| | | LWSDSRWAQELKPKLSNETRRKLEDAKHDLQRANPEWQRL |
| | | AKRKQEYSRHLANTVLSMAREYTACETVVIAIENLPMKGGFV |
| | | DGNGSRESGWDNFFTHKKENRWMIKDIHKALSDLAPNRGVH |
| | | VLEVNPQYTSQTCPECGHRDKANRDPIQRERFCCTHCGAQRH |
| | | ADLEVATHNIAMVATTGKSLTGKSLAPQRLQ |
| CasΦ.42 | SEQ ID | LEIPEGEPGHVPWFQRMDIPEGQIGHVNKIQRFNFVHGKNSGK |
| | NO: 264 | VKFSDKTGRVKRYHHSKYKDATKPYKFLEESKKVSALDSILA |
| | | IITIGDDWVVFDIRGLYRNVFYRELAQKGLTAVQLLDLFTGDP |
| | | VIDPKKGIITFSYKEGVVPVFSQKIVSRFKSRDTLEKLTSQGPV |
| | | ALLSVDLGQNEPVAARVCSLKNINDKIALDNSCRIPFLDDYKK |
| | | QIKDYRDSLDELEIKIRLEAINSLDVNQQVEIRDLDVFSADRA |
| | | KASTVDMFDIDPNLISWDSMSDARFSTQISDLYLKNGGDESR |
| | | VYFEINNKRIKRSDYNISQLVRPKLSDSTRKNLNDSIWKLKRT |
| | | SEEYLKLSKRKLELSRAVVNYTIRQSKLLSGINDIVIILEDLDV |
| | | KKKFNGRGIRDIGWDNFFSSRKENRWFIPAFHKSFSELSSNRG |
| | | LCVIEVNPAWTSATCPDCGFCSKENRDGINFTCRKCGVSYHA |
| | | DIDVATLNIARVAVLGKPMSGPADRERLGGTKKPRVARSRKD |
| | | MKRKDISNGTVEVMVTA |
| CasΦ.46 | SEQ ID | IPSFGYLDRLKIAKGQPGYIPEWQRETINPSKKVRRYWATNHE |
| | NO: 265 | KIRNAIPLVVFIGDDWVIIDGRGLLRDARRRKLADKNTTIEQL |
| | | LEMVSNDPVIDSTRGIATLSYVEGVVPVRSFIPIGEKKGREYLE |
| | | KSTQKESVTLLSVDIGQINPVSCGVYKVSNGCSKIDFLDKFFL |
| | | DKKHLDAIQKYRTLQDSLEASIVNEALDEIDPSFKKEYQNINS |
| | | QTSNDVKKSLCTEYNIDPEAISWQDITAHSTLISDYLIDNNITN |
| | | DVYRTVNKAKYKTNDFGWYKKFSAKLSKEAREALNEKIWEL |
| | | KIASSKYKKLSVRKKEIARTIANDCVKRAETYGDNVVVAMES |
| | | LTKNNKVMSGRGKRDPGWHNLGQAKVENRWFIQAISSAFED |
| | | KATHHGTPVLKVNPAYTSQTCPSCGHCSKDNRSSKDRTIFVC |
| | | KSCGEKFNADLDVATYNIAHVAFSGKKLSPPSEKSSATKKPRS |
| | | ARKSKKSRKS |
| CasΦ.47 | SEQ ID | SPIEKLLNGLLVKITFGNDWIICDARGLLDNVQKGIIHKSYFTN |
| | NO: 266 | KSSLVDLIDLFTCNPIVNYKNNVVTFCYKEGVVDVKSFTPIKS |
| | | GPKTQENLIKKLKYSRFQNEKDACVLGVGVDVGVTNPFAING |
| | | FKMPVDESSEWVMLNEPLFTIETSQAFREEIMAYQQRTDEMN |
| | | DQFNQQSIDLLPPEYKVEFDNLPEDINEVAKYNLLHTLNIPNN |
| | | FLWDKMSNTTQFISDYLIQIGRGTETEKTITTKKGKEKILTIRD |
| | | VNWFNTFKPKISEETGKARTEIKRDLQKNSDQFQKLAKSREQ |
| | | SCRTWVNNVTEEAKIKSGCPLIIFVIEALVKDNRVFSGKGHRA |
| | | IGWHNFGKQKNERRWWVQAIHKAFQEQGVNHGYPVILCPPQ |
| | | YTSQTCPKCNHVDRDNRSGEKFKCLKYGWIGNADLDVGAY |
| | | NIARVAITGKALSKPLEQKKIKKAKNKT |
| CasΦ.48 | SEQ ID | LLDNVQKGIIHKSYFTNKSSLVDLIDLFTCNPIVNYKNNVVTF |
| | NO: 267 | CYKEGVVDVKSFTPIKSGPKTQENLIKKLKYSRFQNEKDACV |
| | | LGVGVDVGVTNPFAINGFKMPVDESSEWVMLNEPLFTIETSQ |
| | | AFREEIMAYQQRTDEMNDQFNQQSIDLLPPEYKVEFDNLPEDI |
| | | NEVAKYNLLHTLNIPNNFLWDKMSNTTQFISDYLIQIGRGTET |
| | | EKTITTKKGKEKILTIRDVNWFNTFKPKISEETGKARTEIKRDL |
| | | QKNSDQFQKLAKSREQSCRTWVNNVTEEAKIKSGCPLIIFVIE |
| | | ALVKDNRVFSGKGHRAIGWHNFGKQKNERRWWVQAIHKAF |
| | | QEQGVNHGYPVILCPPQYTSQTCPKCNHVDRDNRSGEKFKCL |
| | | KYGWIGNADLDVGAYNIARVAITGKALSKPLEQKKIKKAKN |
| | | KT |
| CasΦ.49 | SEQ ID | MIKPTVSQFLTPGFKLIRNHSRTAGLKLKNEGEEACKKFVREN |
| | NO: 268 | EIPKDECPNFQGGPAIANIIAKSREFTEWEIYQSSLAIQEVIFTLP |
| | | KDKLPEPILKEEWRAQWLSEHGLDTVPYKEAAGLNLIIKNAV |
| | | NTYKGVQVKVDNKNKNNLAKINRKNEIAKLNGEQEISFEEIK |
| | | AFDDKGYLLQKPSPNKSIYCYQSVSPKPFITSKYHNVNLPEEYI |
| | | GYYRKSNEPIVSPYQFDRLRIPIGEPGYVPKWQYTFLSKKENK |
| | | RRKLSKRIKNVSPILGIICIKKDWCVFDMRGLLRTNHWKKYH |
| | | KPTDSINDLFDYFTGDPVIDTKANVVRFRYKMENGIVNYKPV |
| | | REKKGKELLENICDQNGSCKLATVDVGQNNPVAIGLFELKKV |
| | | NGELTKTLISRHPTPIDFCNKITAYRERYDKLESSIKLDAIKQLT |
| | | SEQKIEVDNYNNNFTPQNTKQIVCSKLNINPNDLPWDKMISGT |
| | | HFISEKAQVSNKSEIYFTSTDKGKTKDVMKSDYKWFQDYKPK |
| | | LSKEVRDALSDIEWRLRRESLEFNKLSKSREQDARQLANWISS |
| | | MCDVIGIENLVKKNNFFGGSGKREPGWDNFYKPKKENRWWI |
| | | NAIHKALTELSQNKGKRVILLPAMRTSITCPKCKYCDSKNRN |
| | | GEKFNCLKCGIELNADIDVATENLATVAITAQSMPKPTCERSG |
| | | DAKKPVRARKAKAPEFHDKLAPSYTVVLREAVKRPAATKK |
| | | AGQAKKKKEF |
| | | (Bold sequence is Nuclear Localization Signal) |

In some embodiments, any of the programmable CasΦ nuclease of the present disclosure (e.g., any one of SEQ ID NO: 221-SEQ ID NO: 268 or fragments or variants thereof) may include a nuclear localization signal (NLS). In some cases, said NLS may have a sequence of KRPAATKIKAGQAKKiKKIEF (SEQ ID NO: 269).

A CasΦ polypeptide or a variant thereof can comprise at least 70%, at least 75%, at least 80%, at least 85%, at least 90%, at least 92%, at least 95%, at least 97%, at least 99%, or 100% sequence identity with any one of SEQ ID NO: 221-SEQ ID NO: 268.

In some embodiments, the Type VI CRISPR/Cas enzyme is a programmable Cas13 nuclease. The general architecture of a Cas13 protein includes an N-terminal domain and two HEPN (higher eukaryotes and prokaryotes nucleotide-binding) domains separated by two helical domains (Liu et al., Cell 2017 Jan. 12; 168(1-2):121-134.e12). The HEPN domains each comprise aR-X₄-H motif Shared features across Cas13 proteins include that upon binding of the crRNA of the guide nucleic acid to a target nucleic acid, the protein undergoes a conformational change to bring together the HEPN domains and form a catalytically active RNase. (Tambe et al., Cell Rep. 2018 Jul. 24; 24(4): 1025-1036.). Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the Cas13 proteins cleavage efficiency or mutations that catalytically inactivate the HEPN domains. Programmable Cas13 nucleases consistent with the present disclosure also Cas13 nucleases comprising catalytic

A programmable Cas13 nuclease can be a Cas13a protein (also referred to as “c2c2”), a Cas13b protein, a Cas13c protein, a Cas13d protein, or a Cas13e protein. Example C2c2 proteins are set forth as SEQ ID NO: 153-SEQ ID NO: 160. In some cases, a subject C2c2 protein includes an amino acid sequence having 80% or more (e.g., 85% or more, 90% or more, 95% or more, 98% or more, 99% or more, 99.5% or more, or 100%) amino acid sequence identity with the amino acid sequence set forth in any one of SEQ ID NO: 153-SEQ ID NO: 160. In some cases, a suitable C2c2 polypeptide comprises an amino acid sequence having at least 80%, at least 85%, at least 90%, at least 95%, at least 98%, at least 99%, or 100%, amino acid sequence identity to the Listeria seeligeri C2c2 amino acid sequence set forth in SEQ ID NO: 153. In some cases, a suitable C2c2 polypeptide comprises an amino acid sequence having at least 80%, at least 85%, at least 90%, at least 95%, at least 98%, at least 99%, or 100%, amino acid sequence identity to the Leptotrichia buccalis C2c2 amino acid sequence set forth in SEQ ID NO: 154. In some cases, a suitable C2c2 polypeptide comprises an amino acid sequence having at least 80%, at least 85%, at least 90%, at least 95%, at least 98%, at least 99%, or 100%, amino acid sequence identity to the Rhodobacter capsulatus C2c2 amino acid sequence set forth in SEQ ID NO: 156. In some cases, a suitable C2c2 polypeptide comprises an amino acid sequence having at least 80%, at least 85%, at least 90%, at least 95%, at least 98%, at least 99%, or 100%, amino acid sequence identity to the Carnobacterium gallinarum C2c2 amino acid sequence set forth in SEQ ID NO: 157. In some cases, a suitable C2c2 polypeptide comprises an amino acid sequence having at least 80%, at least 85%, at least 90%, at least 95%, at least 98%, at least 99%, or 100%, amino acid sequence identity to the Herbinix hemicellulosilytica C2c2 amino acid sequence set forth in SEQ ID NO: 158. In some cases, the C2c2 protein includes an amino acid sequence having 80% or more amino acid sequence identity with the Leptotrichia buccalis (Lbu) C2c2 amino acid sequence set forth in SEQ ID NO: 154. In some cases, the C2c2 protein is a Leptotrichia buccalis (Lbu) C2c2 protein (e.g., see SEQ ID NO: 154). In some cases, the C2c2 protein includes the amino acid sequence set forth in any one of SEQ ID NO: 153, SEQ ID NO: 154 and SEQ ID NO: 156-SEQ ID NO: 160. In some cases, a C2c2 protein used in a method of the present disclosure is not a Leptotrichia shahii (Lsh) C2c2 protein. In some cases, a C2c2 protein used in a method of the present disclosure is not a C2c2 polypeptide having at least 80%, at least 85%, at least 90%, at least 95%, at least 98%, at least 99%, or 100%, amino acid sequence identity to the Lsh C2c2 polypeptide set forth in SEQ ID NO: 155. Other Cas13 protein sequences are set forth in SEQ ID NO: 153-SEQ ID NO: 170.

TABLE 4
Cas13 Protein Sequences

| SEQ | | |
|---|---|---|
| ID | | |
| NO | Description | Sequence |
| SEQ | Listeria | MWISIKTLIHHLGVLFFCDYMYNRREKKIIEVKTMRITKVEVDRKKVLIS |
| ID | seeligeri | RDKNGGKLVYENEMQDNTEQIMHHKKSSFYKSVVNKTICRPEQKQMK |
| NO: | C2c2 amino | KLVHGLLQENSQEKIKVSDVTKLNISNFLNHRFKKSLYYFPENSPDKSE |
| 153 | acid | EYRIEINLSQLLEDSLKKQQGTFICWESFSKDMELYINWAENYISSKTKL |
| | sequence | IKKSIRNNRIQSTESRSGQLMDRYMKDILNKNKPFDIQSVSEKYQLEKLT |
| | | SALKATFKEAKKNDKEINYKLKSTLQNHERQIIEELKENSELNQFNIEIR |
| | | KHLETYFPIKKTNRKVGDIRNLEIGEIQKIVNHRLKNKIVQRILQEGKLA |
| | | SYEIESTVNSNSLQKIKIEEAFALKFINACLFASNNLRNMVYPVCKKDIL |
| | | MIGEFKNSFKEIKHKKFIRQWSQFFSQEITVDDIELASWGLRGAIAPIRNE |
| | | IIHLKKHSWKKFFNNPTFKVKKSKIINGKTKDVTSEFLYKETLFKDYFYS |
| | | ELDSVPELIINKMESSKILDYYSSDQLNQVFTIPNFELSLLTSAVPFAPSFK |
| | | RVYLKGFDYQNQDEAQPDYNLKLNIYNEKAFNSEAFQAQYSLFKMVY |
| | | YQVFLPQFTTNNDLFKSSVDFILTLNKERKGYAKAFQDIRKMNKDEKPS |
| | | EYMSYIQSQLMLYQKKQEEKEKINHFEKFINQVFIKGFNSFIEKNRLTYI |
| | | CHPTKNTVPENDNIEIPFHTDMDDSNIAFWLMCKLLDAKQLSELRNEMI |
| | | KFSCSLQSTEEISTFTKAREVIGLALLNGEKGCNDWKELFDDKEAWKK |
| | | NMSLYVSEELLQSLPYTQEDGQTPVINRSIDLVKKYGTETILEKLFSSSD |
| | | DYKVSAKDIAKLHEYDVTEKIAQQESLHKQWIEKPGLARDSAWTKKY |
| | | QNVINDISNYQWAKTKVELTQVRHLHQLTIDLLSRLAGYMSIADRDFQ |
| | | FSSNYILERENSEYRVTSWILLSENKNKNKYNDYELYNLKNASIKVSSK |
| | | NDPQLKVDLKQLRLTLEYLELFDNRLKEKRNNISHFNYLNGQLGNSILE |
| | | LFDDARDVLSYDRKLKNAVSKSLKEILSSHGMEVTFKPLYQTNHHLKI |
| | | DKLQPKKIHHLGEKSTVSSNQVSNEYCQLVRTLLTMK |
| SEQ | Leptotrichia | MKVTKVGGISHKKYTSEGRLVKSESEENRTDERLSALLNMRLDMYIKN |
| ID | buccalis | PSSTETKENQKRIGKLKKFFSNKMVYLKDNTLSLKNGKKENIDREYSET |
| NO: | (Lbu) C2c2 | DILESDVRDKKNFAVLKKIYLNENVNSEELEVFRNDIKKKLNKINSLKY |
| 154 | amino acid | SFEKNKANYQKINENNIEKVEGKSKRNIIYDYYRESAKRDAYVSNVKE |
| | sequence | AFDKLYKEEDIAKLVLEIENLTKLEKYKIREFYHEIIGRKNDKENFAKIIY |
| | | EEIQNVNNMKELIEKVPDMSELKKSQVFYKYYLDKEELNDKNIKYAFC |
| | | HFVEIEMSQLLKNYVYKRLSNISNDKIKRIFEYQNLKKLIENKLLNKLDT |
| | | YVRNCGKYNYYLQDGEIATSDFIARNRQNEAFLRNIIGVSSVAYFSLRNI |
| | | LETENENDITGRMRGKTVKNNKGEEKYVSGEVDKIYNENKKNEVKEN |
| | | LKMFYSYDFNMDNKNEIEDFFANIDEAISSIRHGIVHFNLELEGKDIFAF |
| | | KNIAPSEISKKMFQNEINEKKLKLKIFRQLNSANVFRYLEKYKILNYLKR |
| | | TRFEFVNKNIPFVPSFTKLYSRIDDLKNSLGIYWKTPKTNDDNKTKEIID |
| | | AQIYLLKNIYYGEFLNYFMSNNGNFFEISKEIIELNKNDKRNLKTGFYKL |
| | | QKFEDIQEKIPKEYLANIQSLYMINAGNQDEEEKDTYIDFIQKIFLKGFM |
| | | TYLANNGRLSLIYIGSDEETNTSLAEKKQEFDKFLKKYEQNNNIKIPYEI |
| | | NEFLREIKLGNILKYTERLNMFYLILKLLNHKELTNLKGSLEKYQSANK |
| | | EEAFSDQLELINLLNLDNNRVTEDFELEADEIGKFLDFNGNKVKDNKEL |
| | | KKFDTNKIYFDGENIIKHRAFYNIKKYGMLNLLEKIADKAGYKISIEELK |
| | | KYSNKKNEIEKNHKMQENLHRKYARPRKDEKFTDEDYESYKQAIENIE |
| | | EYTHLKNKVEFNELNLLQGLLLRILHRLVGYTSIWERDLRFRLKGEFPE |
| | | NQYIEEIFNFENKKNVKYKGGQIVEKYIKFYKELHQNDEVKINKYSSAN |
| | | IKVLKQEKKDLYIRNYIAHFNYIPHAEISLLEVLENLRKLLSYDRKLKNA |
| | | VMKSVVDILKEYGFVATFKIGADKKIGIQTLESEKIVHLKNLKKKKLMT |
| | | DRNSEELCKLVKIMFEYKMEEKKSEN |
| SEQ | Leptotrichia | MGNLFGHKRWYEVRDKKDFKIKRKVKVKRNYDGNKYILNINENNNKE |
| ID | shahii (Lsh) | KIDNNKFIRKYINYKKNDNILKEFTRKFHAGNILFKLKGKEGIIRIENND |
| NO: | C2c2 | DFLETEEVVLYIEAYGKSEKLKALGITKKKIIDEAIRQGITKDDKKIEIKR |
| 155 | protein | QENEEEIEIDIRDEYTNKTLNDCSIILRIIENDELETKKSIYEIFKNINMSLY |
| | | KIIEKIIENETEKVFENRYYEEHLREKLLKDDKIDVILTNFMEIREKIKSNL |
| | | EILGFVKFYLNVGGDKKKSKNKKMLVEKILNINVDLTVEDIADFVIKEL |
| | | EFWNITKRIEKVKKVNNEFLEKRRNRTYIKSYVLLDKHEKFKIERENKK |
| | | DKIVKFFVENIKNNSIKEKIEKILAEFKIDELIKKLEKELKKGNCDTEIFGI |
| | | FKKHYKVNFDSKKFSKKSDEEKELYKIIYRYLKGRIEKILVNEQKVRLK |
| | | KMEKIEIEKILNESILSEKILKRVKQYTLEHIMYLGKLRHNDIDMTTVNT |
| | | DDFSRLHAKEELDLELITFFASTNMELNKIFSRENINNDENIDFFGGDRE |
| | | KNYVLDKKILNSKIKIIRDLDFIDNKNNITNNFIRKFTKIGTNERNRILHAI |
| | | SKERDLQGTQDDYNKVINIIQNLKISDEEVSKALNLDVVFKDKKNIITKI |
| | | NDIKISEENNNDIKYLPSFSKVLPEILNLYRNNPKNEPFDTIETEKIVLNA |
| | | LIYVNKELYKKLILEDDLEENESKNIFLQELKKTLGNIDEIDENIIENYYK |
| | | NAQISASKGNNKAIKKYQKKVIECYIGYLRKNYEELFDFSDFKMNIQEI |
| | | KKQIKDINDNKTYERITVKTSDKTIVINDDFEYIISIFALLNSNAVINKIRN |
| | | RFFATSVWLNTSEYQNIIDILDEIMQLNTLRNECITENWNLNLEEFIQKM |
| | | KEIEKDFDDFKIQTKKEIFNNYYEDIKNNILTEFKDDINGCDVLEKKLEKI |
| | | VIFDDETKFEIDKKSNILQDEQRKLSNINKKDLKKKVDQYIKDKDQEIKS |
| | | KILCRIIFNSDFLKKYKKEIDNLIEDMESENENKFQEIYYPKERKNELYIY |
| | | KKNLFLNIGNPNFDKIYGLISNDIKMADAKFLFNIDGKNIRKNKISEIDAI |
| | | LKNLNDKLNGYSKEYKEKYIKKLKENDDFFAKNIQNKNYKSFEKDYN |
| | | RVSEYKKIRDLVEFNYLNKIESYLIDINWKLAIQMARFERDMHYIVNGL |
| | | RELGIIKLSGYNTGISRAYPKRNGSDGFYTTTAYYKFFDEESYKKFEKIC |
| | | YGFGIDLSENSEINKPENESIRNYISHFYIVRNPFADYSIAEQIDRVSNLLS |
| | | YSTRYNNSTYASVFEVFKKDVNLDYDELKKKFKLIGNNDILERLMKPK |
| | | KVSVLELESYNSDYIKNLIIELLTKIENTNDTL |
| SEQ | Rhodobacter | MQIGKVQGRTISEFGDPAGGLKRKISTDGKNRKELPAHLSSDPKALIGQ |
| ID | capsulatus | WISGIDKIYRKPDSRKSDGKAIHSPTPSKMQFDARDDLGEAFWKLVSEA |
| NO: | C2c2 amino | GLAQDSDYDQFKRRLHPYGDKFQPADSGAKLKFEADPPEPQAFHGRW |
| 156 | acid | YGAMSKRGNDAKELAAALYEHLHVDEKRIDGQPKRNPKTDKFAPGLV |
| | sequence | VARALGIESSVLPRGMARLARNWGEEEIQTYFVVDVAASVKEVAKAA |
| | | VSAAQAFDPPRQVSGRSLSPKVGFALAEHLERVTGSKRCSFDPAAGPSV |
| | | LALHDEVKKTYKRLCARGKNAARAFPADKTELLALMRHTHENRVRNQ |
| | | MVRMGRVSEYRGQQAGDLAQSHYWTSAGQTEIKESEIFVRLWVGAFA |
| | | LAGRSMKAWIDPMGKIVNTEKNDRDLTAAVNIRQVISNKEMVAEAMA |
| | | RRGIYFGETPELDRLGAEGNEGFVFALLRYLRGCRNQTFHLGARAGFL |
| | | KEIRKELEKTRWGKAKEAEHVVLTDKTVAAIRAIIDNDAKALGARLLA |
| | | DLSGAFVAHYASKEHFSTLYSEIVKAVKDAPEVSSGLPRLKLLLKRADG |
| | | VRGYVHGLRDTRKHAFATKLPPPPAPRELDDPATKARYIALLRLYDGPF |
| | | RAYASGITGTALAGPAARAKEAATALAQSVNVTKAYSDVMEGRSSRL |
| | | RPPNDGETLREYLSALTGETATEFRVQIGYESDSENARKQAEFIENYRR |
| | | DMLAFMFEDYIRAKGFDWILKIEPGATAMTRAPVLPEPIDTRGQYEHW |
| | | QAALYLVMHFVPASDVSNLLHQLRKWEALQGKYELVQDGDATDQAD |
| | | ARREALDLVKRFRDVLVLFLKTGEARFEGRAAPFDLKPFRALFANPATF |
| | | DRLFMATPTTARPAEDDPEGDGASEPELRVARTLRGLRQIARYNHMAV |
| | | LSDLFAKHKVRDEEVARLAEIEDETQEKSQIVAAQELRTDLHDKVMKC |
| | | HPKTISPEERQSYAAAIKTIEEHRFLVGRVYLGDHLRLHRLMMDVIGRLI |
| | | DYAGAYERDTGTFLINASKQLGAGADWAVTIAGAANTDARTQTRKDL |
| | | AHFNVLDRADGTPDLTALVNRAREMMAYDRKRKNAVPRSILDMLARL |
| | | GLTLKWQMKDHLLQDATITQAAIKHLDKVRLTVGGPAAVTEARFSQD |
| | | YLQMVAAVFNGSVQNPKPRRRDDGDAWHKPPKPATAQSQPDQKPPN |
| | | KAPSAGSRLPPPQVGEVYEGVVVKVIDTGSLGFLAVEGVAGNIGLHISR |
| | | LRRIREDAIIVGRRYRFRVEIYVPPKSNTSKLNAADLVRID |
| SEQ | Carnobacterium | MRITKVKIKLDNKLYQVTMQKEEKYGTLKLNEESRKSTAEILRLKKASF |
| ID | gallinarum | NKSFHSKTINSQKENKNATIKKNGDYISQIFEKLVGVDTNKNIRKPKMS |
| NO: | C2c2 amino | LTDLKDLPKKDLALFIKRKFKNDDIVEIKNLDLISLFYNALQKVPGEHFT |
| 157 | acid | DESWADFCQEMMPYREYKNKFIERKIILLANSIEQNKGFSINPETFSKRK |
| | sequence | RVLHQWAIEVQERGDFSILDEKLSKLAEIYNFKKMCKRVQDELNDLEK |
| | | SMKKGKNPEKEKEAYKKQKNFKIKTIWKDYPYKTHIGLIEKIKENEELN |
| | | QFNIEIGKYFEHYFPIKKERCTEDEPYYLNSETIATTVNYQLKNALISYL |
| | | MQIGKYKQFGLENQVLDSKKLQEIGIYEGFQTKFMDACVFATSSLKNII |
| | | EPMRSGDILGKREFKEAIATSSFVNYHHFFPYFPFELKGMKDRESELIPF |
| | | GEQTEAKQMQNIWALRGSVQQIRNEIFHSFDKNQKFNLPQLDKSNFEF |
| | | DASENSTGKSQSYIETDYKFLFEAEKNQLEQFFIERIKSSGALEYYPLKSL |
| | | EKLFAKKEMKFSLGSQVVAFAPSYKKLVKKGHSYQTATEGTANYLGL |
| | | SYYNRYELKEESFQAQYYLLKLIYQYVFLPNFSQGNSPAFRETVKAILRI |
| | | NKDEARKKMKKNKKFLRKYAFEQVREMEFKETPDQYMSYLQSEMRE |
| | | EKVRKAEKNDKGFEKNITMNFEKLLMQIFVKGFDVFLTTFAGKELLLSS |
| | | EEKVIKETEISLSKKINEREKTLKASIQVEHQLVATNSAISYWLFCKLLDS |
| | | RHLNELRNEMIKFKQSRIKFNHTQHAELIQNLLPIVELTILSNDYDEKND |
| | | SQNVDVSAYFEDKSLYETAPYVQTDDRTRVSFRPILKLEKYHTKSLIEA |
| | | LLKDNPQFRVAATDIQEWMHKREEIGELVEKRKNLHTEWAEGQQTLG |
| | | AEKREEYRDYCKKIDRFNWKANKVTLTYLSQLHYLITDLLGRMVGFSA |
| | | LFERDLVYFSRSFSELGGETYHISDYKNLSGVLRLNAEVKPIKIKNIKVID |
| | | NEENPYKGNEPEVKPFLDRLHAYLENVIGIKAVHGKIRNQTAHLSVLQL |
| | | ELSMIESMNNLRDLMAYDRKLKNAVTKSMIKILDKHGMILKLKIDENH |
| | | KNFEIESLIPKEIIHLKDKAIKTNQVSEEYCQLVLALLTTNPGNQLN |
| SEQ | Herbinix | MKLTRRRISGNSVDQKITAAFYRDMSQGLLYYDSEDNDCTDKVIESMD |
| ID | hemicellu- | FERSWRGRILKNGEDDKNPFYMFVKGLVGSNDKIVCEPIDVDSDPDNL |
| NO: | losilytica | DILINKNLTGFGRNLKAPDSNDTLENLIRKIQAGIPEEEVLPELKKIKEMI |
| 158 | C2c2 amino | QKDIVNRKEQLLKSIKNNRIPFSLEGSKLVPSTKKMKWLFKLIDVPNKTF |
| | acid | NEKMLEKYWEIYDYDKLKANITNRLDKTDKKARSISRAVSEELREYHK |
| | sequence | NLRTNYNRFVSGDRPAAGLDNGGSAKYNPDKEEFLLFLKEVEQYFKKY |
| | | FPVKSKHSNKSKDKSLVDKYKNYCSYKVVKKEVNRSIINQLVAGLIQQ |
| | | GKLLYYFYYNDTWQEDFLNSYGLSYIQVEEAFKKSVMTSLSWGINRLT |
| | | SFFIDDSNTVKFDDITTKKAKEAIESNYFNKLRTCSRMQDHFKEKLAFF |
| | | YPVYVKDKKDRPDDDIENLIVLVKNAIESVSYLRNRTFHFKESSLLELL |
| | | KELDDKNSGQNKIDYSVAAEFIKRDIENLYDVFREQIRSLGIAEYYKAD |
| | | MISDCFKTCGLEFALYSPKNSLMPAFKNVYKRGANLNKAYIRDKGPKE |
| | | TGDQGQNSYKALEEYRELTWYIEVKNNDQSYNAYKNLLQLIYYHAFLP |
| | | EVRENEALITDFINRTKEWNRKETEERLNTKNNKKHKNFDENDDITVN |
| | | TYRYESIPDYQGESLDDYLKVLQRKQMARAKEVNEKEEGNNNYIQFIR |
| | | DVVVWAFGAYLENKLKNYKNELQPPLSKENIGLNDTLKELFPEEKVKS |
| | | PFNIKCRFSISTFIDNKGKSTDNTSAEAVKTDGKEDEKDKKNIKRKDLLC |
| | | FYLFLRLLDENEICKLQHQFIKYRCSLKERRFPGNRTKLEKETELLAELE |
| | | ELMELVRFTMPSIPEISAKAESGYDTMIKKYFKDFIEKKVFKNPKTSNLY |
| | | YHSDSKTPVTRKYMALLMRSAPLHLYKDIFKGYYLITKKECLEYIKLSN |
| | | IIKDYQNSLNELHEQLERIKLKSEKQNGKDSLYLDKKDFYKVKEYVENL |
| | | EQVARYKHLQHKINFESLYRIFRIHVDIAARMVGYTQDWERDMHFLFK |
| | | ALVYNGVLEERRFEAIFNNNDDNNDGRIVKKIQNNLNNKNRELVSMLC |
| | | WNKKLNKNEFGAIIWKRNPIAHLNHFTQTEQNSKSSLESLINSLRILLAY |
| | | DRKRQNAVTKTINDLLLNDYHIRIKWEGRVDEGQIYFNIKEKEDIENEPI |
| | | IHLKHLHKKDCYIYKNSYMFDKQKEWICNGIKEEVYDKSILKCIGNLFK |
| | | FDYEDKNKSSANPKHT |
| SEQ | Paludibacter | MRVSKVKVKDGGKDKMVLVHRKTTGAQLVYSGQPVSNETSNILPEKK |
| ID | propionicigenes | RQSFDLSTLNKTIIKFDTAKKQKLNVDQYKIVEKIFKYPKQELPKQIKAE |
| NO: | C2c2 | EILPFLNHKFQEPVKYWKNGKEESFNLTLLIVEAVQAQDKRKLQPYYD |
| 159 | amino acid | WKTWYIQTKSDLLKKSIENNRIDLTENLSKRKKALLAWETEFTASGSID |
| | sequence | LTHYHKVYMTDVLCKMLQDVKPLTDDKGKINTNAYHRGLKKALQNH |
| | | QPAIFGTREVPNEANRADNQLSIYHLEVVKYLEHYFPIKTSKRRNTADDI |
| | | AHYLKAQTLKTTIEKQLVNAIRANIIQQGKTNHHELKADTTSNDLIRIKT |
| | | NEAFVLNLTGTCAFAANNIRNMVDNEQTNDILGKGDFIKSLLKDNTNS |
| | | QLYSFFFGEGLSTNKAEKETQLWGIRGAVQQIRNNVNHYKKDALKTVF |
| | | NISNFENPTITDPKQQTNYADTIYKARFINELEKIPEAFAQQLKTGGAVS |
| | | YYTIENLKSLLTTFQFSLCRSTIPFAPGFKKVFNGGINYQNAKQDESFYE |
| | | LMLEQYLRKENFAEESYNARYFMLKLIYNNLFLPGFTTDRKAFADSVG |
| | | FVQMQNKKQAEKVNPRKKEAYAFEAVRPMTAADSIADYMAYVQSEL |
| | | MQEQNKKEEKVAEETRINFEKFVLQVFIKGFDSFLRAKEFDFVQMPQPQ |
| | | LTATASNQQKADKLNQLEASITADCKLTPQYAKADDATHIAFYVFCKL |
| | | LDAAHLSNLRNELIKFRESVNEFKFHHLLEIIEICLLSADVVPTDYRDLYS |
| | | SEADCLARLRPFIEQGADITNWSDLFVQSDKHSPVIHANIELSVKYGTTK |
| | | LLEQIINKDTQFKTTEANFTAWNTAQKSIEQLIKQREDHHEQWVKAKN |
| | | ADDKEKQERKREKSNFAQKFIEKHGDDYLDICDYINTYNWLDNKMHF |
| | | VHLNRLHGLTIELLGRMAGFVALFDRDFQFFDEQQIADEFKLHGFVNL |
| | | HSIDKKLNEVPTKKIKEIYDIRNKIIQINGNKINESVRANLIQFISSKRNYY |
| | | NNAFLHVSNDEIKEKQMYDIRNHIAHFNYLTKDAADFSLIDLINELRELL |
| | | HYDRKLKNAVSKAFIDLFDKHGMILKLKLNADHKLKVESLEPKKIYHL |
| | | GSSAKDKPEYQYCTNQVMMAYCNMCRSLLEMKK |
| SEQ | Leptotrichia | MYMKITKIDGVSHYKKQDKGILKKKWKDLDERKQREKIEARYNKQIES |
| ID | wadei (Lwa) | KIYKEFFRLKNKKRIEKEEDQNIKSLYFFIKELYLNEKNEEWELKNINLEI |
| NO: | C2c2 amino | LDDKERVIKGYKFKEDVYFFKEGYKEYYLRILFNNLIEKVQNENREKV |
| 160 | acid | RKNKEFLDLKEIFKKYKNRKIDLLLKSINNNKINLEYKKENVNEEIYGIN |
| | sequence | PTNDREMTFYELLKEIIEKKDEQKSILEEKLDNFDITNFLENIEKIFNEETE |
| | | INIIKGKVLNELREYIKEKEENNSDNKLKQIYNLELKKYIENNFSYKKQK |
| | | SKSKNGKNDYLYLNFLKKIMFIEEVDEKKEINKEKFKNKINSNFKNLFV |
| | | QHILDYGKLLYYKENDEYIKNTGQLETKDLEYIKTKETLIRKMAVLVSF |
| | | AANSYYNLFGRVSGDILGTEVVKSSKTNVIKVGSHIFKEKMLNYFFDFE |
| | | IFDANKIVEILESISYSIYNVRNGVGHFNKLILGKYKKKDINTNKRIEEDL |
| | | NNNEEIKGYFIKKRGEIERKVKEKFLSNNLQYYYSKEKIENYFEVYEFEI |
| | | LKRKIPFAPNFKRIIKKGEDLFNNKNNKKYEYFKNFDKNSAEEKKEFLK |
| | | TRNFLLKELYYNNFYKEFLSKKEEFEKIVLEVKEEKKSRGNINNKKSGV |
| | | SFQSIDDYDTKINISDYIASIHKKEMERVEKYNEEKQKDTAKYIRDFVEE |
| | | IFLTGFINYLEKDKRLHFLKEEFSILCNNNNNVVDFNININEEKIKEFLKE |
| | | NDSKTLNLYLFFNMIDSKRISEFRNELVKYKQFTKKRLDEEKEFLGIKIE |
| | | LYETLIEFVILTREKLDTKKSEEIDAWLVDKLYVKDSNEYKEYEEILKLF |
| | | VDEKILSSKEAPYYATDNKTPILLSNFEKTRKYGTQSFLSEIQSNYKYSK |
| | | VEKENIEDYNKKEEIEQKKKSNIEKLQDLKVELHKKWEQNKITEKEIEK |
| | | YNNTTRKINEYNYLKNKEELQNVYLLHEMLSDLLARNVAFFNKWERD |
| | | FKFIVIAIKQFLRENDKEKVNEFLNPPDNSKGKKVYFSVSKYKNTVENID |
| | | GIHKNFMNLIFLNNKFMNRKIDKMNCAIWVYFRNYIAHFLHLHTKNEK |
| | | ISLISQMNLLIKLFSYDKKVQNHILKSTKTLLEKYNIQINFEISNDKNEVF |
| | | KYKIKNRLYSKKGKMLGKNNKFEILENEFLENVKAMLEYSE |
| SEQ | Bergeyella | MENKTSLGNNIYYNPFKPQDKSYFAGYFNAAMENTDSVFRELGKRLK |
| ID | zoohelcum | GKEYTSENFFDAIFKENISLVEYERYVKLLSDYFPMARLLDKKEVPIKER |
| NO: | Cas13b | KENFKKNFKGIIKAVRDLRNFYTHKEHGEVEITDEIFGVLDEMLKSTVL |
| 161 | | TVKKKKVKTDKTKEILKKSIEKQLDILCQKKLEYLRDTARKIEEKRRNQ |
| | | RERGEKELVAPFKYSDKRDDLIAAIYNDAFDVYIDKKKDSLKESSKAK |
| | | YNTKSDPQQEEGDLKIPISKNGVVFLLSLFLTKQEIHAFKSKIAGFKATVI |
| | | DEATVSEATVSHGKNSICFMATHEIFSHLAYKKLKRKVRTAEINYGEAE |
| | | NAEQLSVYAKETLMMQMLDELSKVPDVVYQNLSEDVQKTFIEDWNEY |
| | | LKENNGDVGTMEEEQVIHPVIRKRYEDKFNYFAIRFLDEFAQFPTLRFQ |
| | | VHLGNYLHDSRPKENLISDRRIKEKITVFGRLSELEHKKALFIKNTETNE |
| | | DREHYWEIFPNPNYDFPKENISVNDKDFPIAGSILDREKQPVAGKIGIKV |
| | | KLLNQQYVSEVDKAVKAHQLKORKASKPSIQNIIEEIVPINESNPKEAIV |
| | | FGGQPTAYLSMNDIHSILYEFFDKWEKKKEKLEKKGEKELRKEIGKELE |
| | | KKIVGKIQAQIQQIIDKDTNAKILKPYQDGNSTAIDKEKLIKDLKQEQNI |
| | | LQKLKDEQTVREKEYNDFIAYQDKNREINKVRDRNHKQYLKDNLKRK |
| | | YPEAPARKEVLYYREKGKVAVWLANDIKRFMPTDFKNEWKGEQHSLL |
| | | QKSLAYYEQCKEELKNLLPEKVFQHLPFKLGGYFQQKYLYQFYTCYLD |
| | | KRLEYISGLVQQAENFKSENKVFKKVENECFKFLKKQNYTHKELDARV |
| | | QSILGYPIFLERGFMDEKPTIIKGKTFKGNEALFADWFRYYKEYQNFQTF |
| | | YDTENYPLVELEKKQADRKRKTKIYQQKKNDVFTLLMAKHIFKSVFKQ |
| | | DSIDQFSLEDLYQSREERLGNQERARQTGERNTNYIWNKTVDLKLCDG |
| | | KITVENVKLKNVGDFIKYEYDQRVQAFLKYEENIEWQAFLIKESKEEEN |
| | | YPYVVEREIEQYEKVRREELLKEVHLIEEYILEKVKDKEILKKGDNQNF |
| | | KYYILNGLLKQLKNEDVESYKVFNLNTEPEDVNINQLKQEATDLEQKA |
| | | FVLTYIRNKFAHNQLPKKEFWDYCQEKYGKIEKEKTYAEYFAEVFKKE |
| | | KEALIK |
| SEQ | Prevotella | MEDDKKTTDSIRYELKDKHFWAAFLNLARHNVYITVNHINKILEEGEIN |
| ID | intermedia | RDGYETTLKNTWNEIKDINKKDRLSKLIIKHFPFLEAATYRLNPTDTTK |
| NO: | Cas13b | QKEEKQAEAQSLESLRKSFFVFIYKLRDLRNHYSHYKHSKSLERPKFEE |
| 162 | | GLLEKMYNIFNASIRLVKEDYQYNKDINPDEDFKHLDRTEEEFNYYFTK |
| | | DNEGNITESGLLFFVSLFLEKKDAIWMQQKLRGFKDNRENKKKMTNEV |
| | | FCRSRMLLPKLRLQSTQTQDWILLDMLNELIRCPKSLYERLREEDREKF |
| | | RVPIEIADEDYDAEQEPFKNTLVRHQDRFPYFALRYFDYNEIFTNLRFQI |
| | | DLGTYHFSIYKKQIGDYKESHHLTHKLYGFERIQEFTKQNRPDEWRKFV |
| | | KTFNSFETSKEPYIPETTPHYHLENQKIGIRFRNDNDKIWPSLKTNSEKN |
| | | EKSKYKLDKSFQAEAFLSVHELLPMMFYYLLLKTENTDNDNEIETKKK |
| | | ENKNDKQEKHKIEEIIENKITEIYALYDTFANGEIKSIDELEEYCKGKDIEI |
| | | GHLPKQMIAILKDEHKVMATEAERKQEEMLVDVQKSLESLDNQINEEI |
| | | ENVERKNSSLKSGKIASWLVNDMMRFQPVQKDNEGKPLNNSKANSTE |
| | | YQLLQRTLAFFGSEHERLAPYFKQTKLIESSNPHPFLKDTEWEKCNNILS |
| | | FYRSYLEAKKNFLESLKPEDWEKNQYFLKLKEPKTKPKTLVQGWKNG |
| | | FNLPRGIFTEPIRKWFMKHRENITVAELKRVGLVAKVIPLFFSEEYKDSV |
| | | QPFYNYHFNVGNINKPDEKNFLNCEERRELLRKKKDEFKKMTDKEKEE |
| | | NPSYLEFKSWNKFERELRLVRNQDIVTWLLCMELFNKKKIKELNVEKIY |
| | | LKNINTNTTKKEKNTEEKNGEEKNIKEKNNILNRIMPMRLPIKVYGREN |
| | | FSKNKKKKIRRNTFFTVYIEEKGTKLLKQGNFKALERDRRLGGLFSFVK |
| | | TPSKAESKSNTISKLRVEYELGEYQKARIEIIKDMLALEKTLIDKYNSLD |
| | | TDNFNKMLTDWLELKGEPDKASFQNDVDLLIAVRNAFSHNQYPMRNR |
| | | IAFANINPFSLSSANTSEEKGLGIANQLKDKTHKTIEKIIEIEKPIETKE |
| SEQ | Prevotella | MQKQDKLFVDRKKNAIFAFPKYITIMENKEKPEPIYYELTDKHFWAAFL |
| ID | buccae | NLARHNVYTTINHINRRLEIAELKDDGYMMGIKGSWNEQAKKLDKKV |
| NO: | Cas 13b | RLRDLIMKHFPFLEAAAYEMTNSKSPNNKEQREKEQSEALSLNNLKNV |
| 163 | | LFIFLEKLQVLRNYYSHYKYSEESPKPIFETSLLKNMYKVFDANVRLVK |
| | | RDYMHHENIDMQRDFTHLNRKKQVGRTKNIIDSPNFHYHFADKEGNM |
| | | TIAGLLFFVSLFLDKKDAIWMQKKLKGFKDGRNLREQMTNEVFCRSRIS |
| | | LPKLKLENVQTKDWMQLDMLNELVRCPKSLYERLREKDRESFKVPFDI |
| | | FSDDYNAEEEPFKNTLVRHQDRFPYFVLRYFDLNEIFEQLRFQIDLGTY |
| | | HFSIYNKRIGDEDEVRHLTHHLYGFARIQDFAPQNQPEEWRKLVKDLD |
| | | HFETSQEPYISKTAPHYHLENEKIGIKFCSAHNNLFPSLQTDKTCNGRSK |
| | | FNLGTQFTAEAFLSVHELLPMMFYYLLLTKDYSRKESADKVEGIIRKEIS |
| | | NIYAIYDAFANNEINSIADLTRRLQNTNILQGHLPKQMISILKGRQKDMG |
| | | KEAERKIGEMIDDTQRRLDLLCKQTNQKIRIGKRNAGLLKSGKIADWL |
| | | VNDMMRFQPVQKDONNIPINNSKANSTEYRMLQRALALFGSENFRLKA |
| | | YFNQMNLVGNDNPHPFLAETQWEHQTNILSFYRNYLEARKKYLKGLK |
| | | PQNWKQYQHFLILKVQKTNRNTLVTGWKNSFNLPRGIFTQPIREWFEK |
| | | HNNSKRIYDQILSFDRVGFVAKAIPLYFAEEYKDNVQPFYDYPFNIGNR |
| | | LKPKKRQFLDKKERVELWQKNKELFKNYPSEKKKTDLAYLDFLSWKK |
| | | FERELRLIKNQDIVTWLMFKELFNMATVEGLKIGEIHLRDIDTNTANEES |
| | | NNILNRIMPMKLPVKTYETDNKGNILKERPLATFYIEETETKVLKQGNF |
| | | KALVKDRRLNGLFSFAETTDLNLEEHPISKLSVDLELIKYQTTRISIFEMT |
| | | LGLEKKLIDKYSTLPTDSFRNMLERWLQCKANRPELKNYVNSLIAVRN |
| | | AFSHNQYPMYDATLFAEVKKFTLFPSVDTKKIELNIAPQLLEIVGKAIKE |
| | | IEKSENKN |
| SEQ | Porphyromonas | MNTVPASENKGQSRTVEDDPQYFGLYLNLARENLIEVESHVRIKFGKK |
| ID | gingivalis | KLNEESLKQSLLCDHLLSVDRWTKVYGHSRRYLPFLHYFDPDSQIEKD |
| NO: | Cas13b | HDSKTGVDPDSAQRLIRELYSLLDFLRNDFSHNRLDGTTFEHLEVSPDIS |
| 164 | | SFITGTYSLACGRAQSRFAVFFKPDDFVLAKNRKEQLISVADGKECLTV |
| | | SGFAFFICLFLDREQASGMLSRIRGFKRTDENWARAVHETFCDLCIRHP |
| | | HDRLESSNTKEALLLDMLNELNRCPRILYDMLPEEERAQFLPALDENSM |
| | | NNLSENSLDEESRLLWDGSSDWAEALTKRIRHQDRFPYLMLRFIEEMD |
| | | LLKGIRFRVDLGEIELDSYSKKVGRNGEYDRTITDHALAFGKLSDFQNE |
| | | EEVSRMISGEASYPVRFSLFAPRYAIYDNKIGYCHTSDPVYPKSKTGEKR |
| | | ALSNPQSMGFISVHDLRKLLLMELLCEGSFSRMQSDFLRKANRILDETA |
| | | EGKLQFSALFPEMRHRFIPPQNPKSKDRREKAETTLEKYKQEIKGRKDK |
| | | LNSQLLSAFDMDQRQLPSRLLDEWMNIRPASHSVKLRTYVKQLNEDCR |
| | | LRLRKFRKDGDGKARAIPLVGEMATFLSQDIVRMIISEETKKLITSAYYN |
| | | EMQRSLAQYAGEENRRQFRAIVAELRLLDPSSGHPFLSATMETAHRYT |
| | | EGFYKCYLEKKREWLAKIFYRPEQDENTKRRISVFFVPDGEARKLLPLLI |
| | | RRRMKEQNDLQDWIRNKQAHPIDLPSHLFDSKVMELLKVKDGKKKW |
| | | NEAFKDWWSTKYPDGMQPFYGLRRELNIHGKSVSYIPSDGKKFADCYT |
| | | HLMEKTVRDKKRELRTAGKPVPPDLAADIKRSFHRAVNEREFMLRLVQ |
| | | EDDRLMLMAINKMMTDREEDILPGLKNIDSILDEENQFSLAVHAKVLE |
| | | KEGEGGDNSLSLVPATIEIKSKRKDWSKYIRYRYDRRVPGLMSHFPEHK |
| | | ATLDEVKTLLGEYDRCRIKIFDWAFALEGAIMSDRDLKPYLHESSSREG |
| | | KSGEHSTLVKMLVEKKGCLTPDESQYLILIRNKAAHNQFPCAAEMPLIY |
| | | RDVSAKVGSIEGSSAKDLPEGSSLVDSLWKKYEMIIRKILPILDPENRFF |
| | | GKLLNNMSQPINDL |
| SEQ | Bacteroides | MESIKNSQKSTGKTLQKDPPYFGLYLNMALLNVRKVENHIRKWLGDV |
| ID | pyogenes | ALLPEKSGFHSLLTTDNLSSAKWTRFYYKSRKFLPFLEMFDSDKKSYEN |
| NO: | Cas13b | RRETAECLDTIDRQKISSLLKEVYGKLQDIRNAFSHYHIDDQSVKHTALI |
| 165 | | ISSEMHRFIENAYSFALQKTRARFTGVFVETDFLQAEEKGDNKKFFAIG |
| | | GNEGIKLKDNALIFLICLFLDREEAFKFLSRATGFKSTKEKGFLAVRETF |
| | | CALCCRQPHERLLSVNPREALLMDMLNELNRCPDILFEMLDEKDQKSF |
| | | LPLLGEEEQAHILENSLNDELCEAIDDPFEMIASLSKRVRYKNRFPYLML |
| | | RYIEEKNLLPFIRFRIDLGCLELASYPKKMGEENNYERSVTDHAMAFGR |
| | | LTDFHNEDAVLQQITKGITDEVRFSLYAPRYAIYNNKIGFVRTSGSDKIS |
| | | FPTLKKKGGEGHCVAYTLQNTKSFGFISIYDLRKILLLSFLDKDKAKNIV |
| | | SGLLEQCEKHWKDLSENLFDAIRTELQKEFPVPLIRYTLPRSKGGKLVSS |
| | | KLADKQEKYESEFERRKEKLTEILSEKDFDLSQIPRRMIDEWLNVLPTSR |
| | | EKKLKGYVETLKLDCRERLRVFEKREKGEHPLPPRIGEMATDLAKDIIR |
| | | MVIDQGVKQRITSAYYSEIQRCLAQYAGDDNRRHLDSIIRELRLKDTKN |
| | | GHPFLGKVLRPGLGHTEKLYQRYFEEKKEWLEATFYPAASPKRVPRFV |
| | | NPPTGKQKELPLIIRNLMKERPEWRDWKQRKNSHPIDLPSQLFENEICRL |
| | | LKDKIGKEPSGKLKWNEMFKLYWDKEFPNGMQRFYRCKRRVEVFDK |
| | | VVEYEYSEEGGNYKKYYEALIDEVVRQKISSSKEKSKLQVEDLTLSVRR |
| | | VFKRAINEKEYQLRLLCEDDRLLFMAVRDLYDWKEAQLDLDKIDNML |
| | | GEPVSVSQVIQLEGGQPDAVIKAECKLKDVSKLMRYCYDGRVKGLMP |
| | | YFANHEATQEQVEMELRHYEDHRRRVFNWVFALEKSVLKNEKLRRFY |
| | | EESQGGCEHRRCIDALRKASLVSEEEYEFLVHIRNKSAHNQFPDLEIGKL |
| | | PPNVTSGFCECIWSKYKAIICRIIPFIDPERRFFGKLLEQK |
| SEQ | Cas 13c | MTEKKSIIFKNKSSVEIVKKDIFSQTPDNMIRNYKITLKISEKNPRVVEAE |
| ID | | IEDLMNSTILKDGRRSARREKSMTERKLIEEKVAENYSLLANCPMEEVD |
| NO: | | SIKIYKIKRFLTYRSNMLLYFASINSFLCEGIKGKDNETEEIWHLKDNDV |
| 166 | | RKEKVKENFKNKLIQSTENYNSSLKNQIEEKEKLLRKESKKGAFYRTIIK |
| | | KLQQERIKELSEKSLTEDCEKIIKLYSELRHPLMHYDYQYFENLFENKEN |
| | | SELTKNLNLDIFKSLPLVRKMKLNNKVNYLEDNDTLFVLQKTKKAKTL |
| | | YQIYDALCEQKNGFNKFINDFFVSDGEENTVFKQIINEKFQSEMEFLEKR |
| | | ISESEKKNEKLKKKFDSMKAHFHNINSEDTKEAYFWDIHSSSNYKTKYN |
| | | ERKNLVNEYTELLGSSKEKKLLREEITQINRKLLKLKQEMEEITKKNSLF |
| | | RLEYKMKIAFGFLFCEFDGNISKFKDEFDASNQEKIIQYHKNGEKYLTY |
| | | FLKEEEKEKFNLEKMQKIIQKTEEEDWLLPETKNNLFKFYLLTYLLLPY |
| | | ELKGDFLGFVKKHYYDIKNVDFMDENQNNIQVSQTVEKQEDYFYHKIR |
| | | LFEKNTKKYEIVKYSIVPNEKLKQYFEDLGIDIKYLTGSVESGEKWLGE |
| | | NLGIDIKYLTVEQKSEVSEEKIKKFL |
| SEQ | Cas 13c | MEKDKKGEKIDISQEMIEEDLRKILILFSRLRHSMVHYDYEFYQALYSG |
| ID | | KDFVISDKNNLENRMISQLLDLNIFKELSKVKLIKDKAISNYLDKNTTIH |
| NO: | | VLGQDIKAIRLLDIYRDICGSKNGFNKFINTMITISGEEDREYKEKVIEHF |
| 167 | | NKKMENLSTYLEKLEKQDNAKRNNKRVYNLLKQKLIEQQKLKEWFGG |
| | | PYVYDIHSSKRYKELYIERKKLVDRHSKLFEEGLDEKNKKELTKINDEL |
| | | SKLNSEMKEMTKLNSKYRLQYKLQLAFGFILEEFDLNIDTFINNFDKDK |
| | | DLIISNFMKKRDIYLNRVLDRGDNRLKNIIKEYKFRDTEDIFCNDRDNNL |
| | | VKLYILMYILLPVEIRGDFLGFVKKNYYDMKHVDFIDKKDKEDKDTFF |
| | | HDLRLFEKNIRKLEITDYSLSSGFLSKEHKVDIEKKINDFINRNGAMKLP |
| | | EDITIEEFNKSLILPIMKNYQINFKLLNDIEISALFKIAKDRSITFKQAIDEI |
| | | KNEDIKKNSKKNDKNNHKDKNINFTQLMKRALHEKIPYKAGMYQIRN |
| | | NISHIDMEQLYIDPLNSYMNSNKNNITISEQIEKIIDVCVTGGVTGKELNN |
| | | NIINDYYMKKEKLVFNLKLRKQNDIVSIESQEKNKREEFVFKKYGLDYK |
| | | DGEINIIEVIQKVNSLQEELRNIKETSKEKLKNKETLFRDISLINGTIRKNI |
| | | NFKIKEMVLDIVRMDEIRHINIHIYYKGENYTRSNIIKFKYAIDGENKKY |
| | | YLKQHEINDINLELKDKFVTLICNMDKHPNKNKQTINLESNYIQNVKFII |
| | | P |
| SEQ | Cas 13c | MENKGNNKKIDFDENYNILVAQIKEYFTKEIENYNNRIDNIIDKKELLKY |
| ID | | SEKKEESEKNKKLEELNKLKSQKLKILTDEEIKADVIKIIKIFSDLRHSLM |
| NO: | | HYEYKYFENLFENKKNEELAELLNLNLFKNLTLLRQMKIENKTNYLEG |
| 168 | | REEFNIIGKNIKAKEVLGHYNLLAEQKNGFNNFINSFFVQDGTENLEFK |
| | | KLIDEHFVNAKKRLERNIKKSKKLEKELEKMEQHYQRLNCAYVWDIHT |
| | | STTYKKLYNKRKSLIEEYNKQINEIKDKEVITAINVELLRIKKEMEEITKS |
| | | NSLFRLKYKMQIAYAFLEIEFGGNIAKFKDEFDCSKMEEVQKYLKKGV |
| | | KYLKYYKDKEAQKNYEFPFEEIFENKDTHNEEWLENTSENNLFKFYILT |
| | | YLLLPMEFKGDFLGVVKKHYYDIKNVDFTDESEKELSQVQLDKMIGDS |
| | | FFHKIRLFEKNTKRYEIIKYSILTSDEIKRYFRLLELDVPYFEYEKGTDEIG |
| | | IFNKNIILTIFKYYQIIFRLYNDLEIHGLFNISSDLDKILRDLKSYGNKNINF |
| | | REFLYVIKQNNNSSTEEEYRKIWENLEAKYLRLHLLTPEKEEIKTKTKEE |
| | | LEKLNEISNLRNGICHLNYKEIIEEILKTEISEKNKEATLNEKIRKVINFIK |
| | | ENELDKVELGFNFINDFFMKKEQFMFGQIKQVKEGNSDSITTERERKEK |
| | | NNKKLKETYELNCDNLSEFYETSNNLRERANSSSLLEDSAFLKKIGLYK |
| | | VKNNKVNSKVKDEEKRIENIKRKLLKDSSDIMGMYKAEVVKKLKEKLI |
| | | LIFKHDEEKRIYVTVYDTSKAVPENISKEILVKRNNSKEEYFFEDNNKKY |
| | | VTEYYTLEITETNELKVIPAKKLEGKEFKTEKNKENKLMLNNHYCFNV |
| | | KIIY |
| SEQ | Cas 13c | MEEIKHKKNKSSIIRVIVSNYDMTGIKEIKVLYQKQGGVDTFNLKTIINL |
| ID | | ESGNLEIISCKPKEREKYRYEFNCKTEINTISITKKDKVLKKEIRKYSLEL |
| NO: | | YFKNEKKDTVVAKVTDLLKAPDKIEGERNHLRKLSSSTERKLLSKTLC |
| 169 | | KNYSEISKTPIEEIDSIKIYKIKRFLNYRSNFLIYFALINDFLCAGVKEDDI |
| | | NEVWLIQDKEHTAFLENRIEKITDYIFDKLSKDIENKKNQFEKRIKKYKT |
| | | SLEELKTETLEKNKTFYIDSIKTKITNLENKITELSLYNSKESLKEDLIKIIS |
| | | IFTNLRHSLMHYDYKSFENLFENIENEELKNLLDLNLFKSIRMSDEFKTK |
| | | NRTNYLDGTESFTIVKKHQNLKKLYTYYNNLCDKKNGFNTFINSFFVT |
| | | DGIENTDFKNLIILHFEKEMEEYKKSIEYYKIKISNEKNKSKKEKLKEKID |
| | | LLQSELINMREHKNLLKQIYFFDIHNSIKYKELYSERKNLIEQYNLQING |
| | | VKDVTAINHINTKLLSLKNKMDKITKQNSLYRLKYKLKIAYSFLMIEFD |
| | | GDVSKFKNNFDPTNLEKRVEYLDKKEEYLNYTAPKNKFNFAKLEEELQ |
| | | KIQSTSEMGADYLNVSPENNLFKFYILTYIMLPVEFKGDFLGFVKNHYY |
| | | NIKNVDFMDESLLDENEVDSNKLNEKIENLKDSSFFNKIRLFEKNIKKYE |
| | | IVKYSVSTQENMKEYFKQLNLDIPYLDYKSTDEIGIFNKNMILPIFKYYQ |
| | | NVFKLCNDIEIHALLALANKKQQNLEYAIYCCSKKNSLNYNELLKTFNR |
| | | KTYQNLSFIRNKIAHLNYKELFSDLFNNELDLNTKVRCLIEFSQNNKFD |
| | | QIDLGMNFINDYYMKKTRFIFNQRRLRDLNVPSKEKIIDGKRKQQNDSN |
| | | NELLKKYGLSRTNIKDIFNKAWY |
| SEQ | Cas 13c | MKVRYRKQAQLDTFIIKTEIVNNDIFIKSIIEKAREKYRYSFLFDGEEKYH |
| ID | | FKNKSSVEIVKNDIFSQTPDNMIRNYKITLKISEKNPRVVEAEIEDLMNS |
| NO: | | TILKDGRRSARREKSMTERKLIEEKVAENYSLLANCPIEEVDSIKIYKIKR |
| 170 | | FLTYRSNMLLYFASINSFLCEGIKGKDNETEEIWHLKDNDVRKEKVKEN |
| | | FKNKLIQSTENYNSSLKNQIEEKEKLSSKEFKKGAFYRTIIKKLQQERIKE |
| | | LSEKSLTEDCEKIIKLYSELRHPLMHYDYQYFENLFENKENSELTKNLN |
| | | LDIFKSLPLVRKMKLNNKVNYLEDNDTLFVLQKTKKAKTLYQIYDALC |
| | | EQKNGFNKFINDFFVSDGEENTVFKQIINEKFQSEMEFLEKRISESEKKN |
| | | EKLKKKLDSMKAHFRNINSEDTKEAYFWDIHSSRNYKTKYNERKNLV |
| | | NEYTKLLGSSKEKKLLREEITKINRQLLKLKQEMEEITKKNSLFRLEYK |
| | | MKIAFGFLFCEFDGNISKFKDEFDASNQEKIIQYHKNGEKYLTSFLKEEE |
| | | KEKFNLEKMQKIIQKTEEEDWLLPETKNNLFKFYLLTYLLLPYELKGDF |
| | | LGFVKKHYYDIKNVDFMDENQNNIQVSQTVEKQEDYFYHKIRLFEKNT |
| | | KKYEIVKYSIVPNEKLKQYFEDLGIDIKYLTGSVESGEKWLGENLGIDIK |
| | | YLTVEQKSEVSEEKNKKVSLKNNGMFNKTILLFVFKYYQIAFKLFNDIE |
| | | LYSLFFLREKSEKPFEVFLEELKDKMIGKQLNFGQLLYVVYEVLVKNK |
| | | DLDKILSKKIDYRKDKSFSPEIAYLRNFLSHLNYSKFLDNFMKINTNKSD |
| | | ENKEVLIPSIKIQKMIQFIEKCNLQNQIDFDFNFVNDFYMRKEKMFFIQL |
| | | KQIFPDINSTEKQKKSEKEEILRKRYHLINKKNEQIKDEHEAQSQLYEKI |
| | | LSLQKIFSCDKNNFYRRLKEEKLLFLEKQGKKKISMKEIKDKIASDISDL |
| | | LGILKKEITRDIKDKLTEKFRYCEEKLLNISFYNHQDKKKEEGIRVFLIRD |
| | | KNSDNFKFESILDDGSNKIFISKNGKEITIQCCDKVLETLMIEKNTLKISS |
| | | NGKIISLIPHYSYSIDVKY |

The programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, Cas14h, Cas14i, Cas14j, or Cas14k. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA.

In some embodiments, a programmable nuclease as disclosed herein is an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA detector nucleic acid and is capable of being activated by a target DNA to initiate trans cleavage of an RNA detector nucleic acid, such as a Type VI CRISPR/Cas enzyme (e.g., Cas13). For example, Cas13a of the present disclosure can be activated by a target RNA to initiate trans cleavage activity of the Cas13a for the cleavage of an RNA detector nucleic acid and can be activated by a target DNA to initiate trans cleavage activity of the Cas13a for trans cleavage of an RNA detector nucleic acid. An RNA detector nucleic acid can be an RNA-based detector nucleic acid molecule. In some embodiments, the Cas13a recognizes and detects ssDNA to initiate transcleavage of RNA detector nucleic acids. Multiple Cas13a isolates can recognize, be activated by, and detect target DNA, including ssDNA, upon hybridization of a guide nucleic acid with the the target DNA. For example, Lbu-Cas13a and Lwa-Cas13a can both be activated to transcollaterally cleave RNA detector nucleic acids by target DNA. Thus, Type VI CRISPR/Cas enzyme (e.g., Cas13, such as Cas13a) can be DNA-activated programmable RNA nucleases, and therefore, can be used to detect a target DNA using the methods as described herein. DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 may exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA may not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target DNA detected by Cas13 disclosed herein can be directly from organisms, or can be indirectly generated by nucleic acid amplification methods, such as PCR and LAMP or any amplification method described herein. Key steps for the sensitive detection of a target DNA, such as a target ssDNA, by a DNA-activated programmable RNA nuclease, such as Cas13a, can include: (1) production or isolation of DNA to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection. The detection of a target DNA by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas protein, with a DNA-activated programmable RNA nuclease, such as a Type VI protein, with a DNA detector nucleic acid and an RNA detector nucleic acid, can enable multiplexed detection of target ssDNAs or a combination of a target dsDNA and a target ssDNA, respectively. Multiplexing of different RNA-activated programmable RNA nucleases that have distinct RNA detector nucleic acid cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein.

Described herein are reagents comprising a single-stranded detector nucleic acid comprising a detection moiety, wherein the detector nucleic acid is capable of being cleaved by the activated nuclease, thereby generating a first detectable signal. As used herein, a detector nucleic acid is used interchangeably with reporter or reporter molecule. In some cases, the detector nucleic acid is a single-stranded nucleic acid comprising deoxyribonucleotides. In other cases, the detector nucleic acid is a single-stranded nucleic acid comprising ribonucleotides. The detector nucleic acid can be a single-stranded nucleic acid comprising at least one deoxyribonucleotide and at least one ribonucleotide. In some cases, the detector nucleic acid is a single-stranded nucleic acid comprising at least one ribonucleotide residue at an internal position that functions as a cleavage site. In some cases, the detector nucleic acid comprises at least 2, 3, 4, 5, 6, 7, 8, 9, or 10 ribonucleotide residues at an internal position. Sometimes the ribonucleotide residues are continuous. Alternatively, the ribonucleotide residues are interspersed in between non-ribonucleotide residues. In some cases, the detector nucleic acid has only ribonucleotide residues. In some cases, the detector nucleic acid has only deoxyribonucleotide residues. In some cases, the detector nucleic acid comprises nucleotides resistant to cleavage by the programmable nuclease described herein. In some cases, the detector nucleic acid comprises synthetic nucleotides. In some cases, the detector nucleic acid comprises at least one ribonucleotide residue and at least one non-ribonucleotide residue. In some cases, detector nucleic acid is 5-20, 5-15, 5-10, 7-20, 7-15, or 7-10 nucleotides in length. In some cases, the detector nucleic acid comprises at least one uracil ribonucleotide. In some cases, the detector nucleic acid comprises at least two uracil ribonucleotides. Sometimes the detector nucleic acid has only uracil ribonucleotides. In some cases, the detector nucleic acid comprises at least one adenine ribonucleotide. In some cases, the detector nucleic acid comprises at least two adenine ribonucleotide. In some cases, the detector nucleic acid has only adenine ribonucleotides. In some cases, the detector nucleic acid comprises at least one cytosine ribonucleotide. In some cases, the detector nucleic acid comprises at least two cytosine ribonucleotide. In some cases, the detector nucleic acid comprises at least one guanine ribonucleotide. In some cases, the detector nucleic acid comprises at least two guanine ribonucleotide. A detector nucleic acid can comprise only unmodified ribonucleotides, only unmodified deoxyribonucleotides, or a combination thereof. In some cases, the detector nucleic acid is from 5 to 12 nucleotides in length. In some cases, the detector nucleic acid is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a detector nucleic acid can be 5, 8, or 10 nucleotides in length. For cleavage by a programmable nuclease comprising Cas12, a detector nucleic acid can be 10 nucleotides in length.

The single-stranded detector nucleic acid comprises a detection moiety capable of generating a first detectable signal. Sometimes the detector nucleic acid comprises a protein capable of generating a signal. A signal can be a calorimetric, potentiometric, amperometric, optical (e.g., fluorescent, colorometric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the detector nucleic acid. Sometimes the detection moiety is at the 3′ terminus of the detector nucleic acid. In some cases, the detection moiety is at the 5′ terminus of the detector nucleic acid. In some cases, the quenching moiety is at the 3′ terminus of the detector nucleic acid. In some cases, the single-stranded detector nucleic acid is at least one population of the single-stranded nucleic acid capable of generating a first detectable signal. In some cases, the single-stranded detector nucleic acid is a population of the single-stranded nucleic acid capable of generating a first detectable signal. Optionally, there is more than one population of single-stranded detector nucleic acid. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded detector nucleic acids capable of generating a detectable signal.

TABLE 5
Exemplary Single Stranded Detector Nucleic Acid

| 5′ Detection Moiety* | Sequence (SEQ ID NO:) | 3′ Quencher* |
|---|---|---|
| /56-FAM/ | rUrUrUrUrU | /3IABKFQ/ |
| /5IRD700/ | rUrUrUrUrU | /3IRQC1N/ |
| /5TYE665/ | rUrUrUrUrU | /3IAbRQSp/ |
| /5Alex594N/ | rUrUrUrUrU | /3IAbRQSp/ |
| /5ATTO633N/ | rUrUrUrUrU | /3IAbRQSp/ |
| /56-FAM/ | rUrUrUrUrUrUrUrU | /3IABKFQ/ |
| /5IRD700/ | rUrUrUrUrUrUrUrU | /3IRQC1N/ |
| /5TYE665/ | rUrUrUrUrUrUrUrU | /3IAbRQSp/ |
| /5Alex594N/ | rUrUrUrUrUrUrUrU | /3IAbRQSp/ |
| /5ATTO633N/ | rUrUrUrUrUrUrUrU | /IAbRQSp/ |
| /56-FAM/ | rUrUrUrUrUrUrUrUrUrU(SEQ ID NO: 3) | /3IABKFQ/ |
| /5IRD700/ | rUrUrUrUrUrUrUrUrUrU(SEQ ID NO: 3) | /3IRQC1N/ |
| /5TYE665/ | rUrUrUrUrUrUrUrUrUrU(SEQ ID NO: 3) | /3IAbRQSp/ |
| /5Alex594N/ | rUrUrUrUrUrUrUrUrUrU(SEQ ID NO: 3) | /3IAbRQSp/ |
| /5ATTO633N/ | rUrUrUrUrUrUrUrUrUrU(SEQ ID NO: 3) | /3IAbRQSp/ |
| /56-FAM/ | TTTTrUrUTTTT(SEQ ID NO: 4) | /3IABKFQ/ |
| /5IRD700/ | TTTTrUrUTTTT(SEQ ID NO: 4) | /3IRQC1N/ |
| /STYE665/ | TTTTrUrUTTTT(SEQ ID NO: 4) | /3IAbRQSp/ |
| /5Alex594N/ | TTTTrUrUTTTT(SEQ ID NO: 4) | /3IAbRQSp/ |
| /5ATTO633N/ | TTTTrUrUTTTT(SEQ ID NO: 4) | /3IAbRQSp/ |
| /56-FAM/ | TTrUrUTT | /3IABKFQ/ |
| /5IRD700/ | TTrUrUTT | /3IRQC1N/ |
| /5TYE665/ | TTrUrUTT | /3IAbRQSp/ |
| /5Alex594N/ | TTrUrUTT | /3IAbRQSp/ |
| /5ATTO633N/ | TTrUrUTT | /3IAbRQSp/ |
| /56-FAM/ | TArArUGC | /3IABKFQ/ |
| /5IRD700/ | TArArUGC | /3IRQC1N/ |
| /5TYE665/ | TArArUGC | /3IAbRQSp/ |
| /5Alex594N/ | TArArUGC | /3IAbRQSp/ |
| /5ATTO633N/ | TArArUGC | /3IAbRQSp/ |
| /56-FAM/ | TArUrGGC | /3IABKFQ/ |
| /5IRD700/ | TArUrGGC | /3IRQC1N/ |
| /5TYE665/ | TArUrGGC | /3IAbRQSp/ |
| /5Alex594N/ | TArUrGGC | /3IAbRQSp/ |
| /5ATTO633N/ | TArUrGGC | /3IAbRQSp/ |
| /56-FAM/ | rUrUrUrUrU | /3IABKFQ/ |
| /5IRD700/ | rUrUrUrUrU | /3IRQC1N/ |
| /5TYE665/ | rUrUrUrUrU | /3IAbRQSp/ |
| /5Alex594N/ | rUrUrUrUrU | /3IAbRQSp/ |
| /5ATTO633N/ | rUrUrUrUrU | /3IAbRQSp/ |
| /56-FAM/ | TTATTATT | /3IABKFQ |
| /56-FAM/ | TTATTATT | /3IABKFQ |
| /5IRD700/ | TTATTATT | /3IRQC1N/ |
| /5TYE665/ | TTATTATT | /3IAbRQSp/ |
| /5Alex594N/ | TTATTATT | /3IAbRQSp/ |
| /5ATTO633N/ | TTATTATT | /3IAbRQSp/ |
| /56-FAM/ | TTTTTT | /3IABKFQ/ |
| /56-FAM/ | TTTTTTTT | /3IABKFQ/ |
| /56-FAM/ | TTTTTTTTTT (SEQ ID NO: 12) | /3IABKFQ/ |
| /56-FAM/ | TTTTTTTTTTTT (SEQ ID NO: 13) | /3IABKFQ/ |
| /56-FAM/ | TTTTTTTTTTTTTT (SEQ ID NO: 14) | /3IABKFQ/ |
| /56-FAM/ | AAAAAA | /3IABKFQ/ |
| /56-FAM/ | CCCCCC | /3IABKFQ/ |
| /56-FAM/ | GGGGGG | /3IABKFQ/ |
| /56-FAM/ | TTATTATT | /3IABKFQ/ |

/56-FAM/: 5′ 6-Fluorescein (Integrated DNA Technologies)
/3IABKFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester)(Integrated DNA Technologies)
/5Alex488N/: 5′ Alexa Fluor 488 (NHS Ester)(Integrated DNA Technologies)
/5ATTO633N/: 5′ ATTO TM 633 (NHS Ester)(Integrated DNA Technologies)
/3IRQC1N/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence at in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 6890 to 690, 690 to 700, 700 to 710, 710 to 720, or 720 to 730 nm. A detection moiety can be a fluorophore that emits a fluorescence in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

A detection moiety can be chosen for use based on the type of sample to be tested. For example, a detection moiety that is an infrared fluorophore is used with a urine sample. As another example, SEQ ID NO: 1 with a fluorophore that emits around 520 nm is used for testing in non-urine samples, and SEQ ID NO: 8 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety emits fluorescence at in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 6890 to 690, 690 to 700, 700 to 710, 710 to 720, or 720 to 730 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nuclease has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorimetric, etc.), or piezo-electric signal. A detector nucleic acid, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorimetric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid.

Often, the protein-nucleic acid is an enzyme-nucleic acid. The enzyme may be sterically hindered when present as in the enzyme-nucleic acid, but then functional upon cleavage from the nucleic acid. Often, the enzyme is an enzyme that produces a reaction with a substrate. An enzyme can be invertase. Often, the substrate of invertase is sucrose and DNS reagent.

Sometimes the protein-nucleic acid is a substrate-nucleic acid. Often the substrate is a substrate that produces a reaction with an enzyme.

A protein-nucleic acid may be attached to a solid support. The solid support, for example, is a surface. A surface can be an electrode. Sometimes the solid support is a bead. Often the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

In some embodiments, the detector nucleic acid comprises a nucleic acid conjugated to an affinity molecule and the affinity molecule conjugated to the fluorophore (e.g., nucleic acid-affinity molecule-fluorophore) or the nucleic acid conjugated to the fluorophore and the fluorophore conjugated to the affinity molecule (e.g., nucleic acid-fluorophore-affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the detector nucleic acid can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicated that no intervening molecules, polypeptides, proteins, or other moieties are present between the two moieties directly conjugated to each other. For example, if a detector nucleic acid comprises a nucleic acid directly conjugated to an affinity molecule and an affinity molecule directly conjugated to a fluorophore—no intervening moiety is present between the nucleic acid and the affinity molecule and no intervening moiety is present between the affinity molecule and the fluorophore. The affinity molecule can be biotin, avidin, streptavidin, or any similar molecule.

In some cases, the reporter comprises a substrate-nucleic acid. The substrate may be sequestered from its cognate enzyme when present as in the substrate-nucleic acid, but then is released from the nucleic acid upon cleavage, wherein the released substrate can contact the cognate enzyme to produce a detectable signal. Often, the substrate is sucrose and the cognate enzyme is invertase, and a DNS reagent can be used to monitor invertase activity.

A major advantage of the devices and methods disclosed herein is the design of excess reporters to total nucleic acids in an unamplified or an amplified sample, not including the nucleic acid of the reporter. Total nucleic acids can include the target nucleic acids and non-target nucleic acids, not including the nucleic acid of the reporter. The non-target nucleic acids can be from the original sample, either lysed or unlysed. The non-target nucleic acids can also be byproducts of amplification. Thus, the non-target nucleic acids can include both non-target nucleic acids from the original sample, lysed or unlysed, and from an amplified sample. The presence of a large amount of non-target nucleic acids, an activated programmable nuclease may be inhibited in its ability to bind and cleave the reporter sequences. This is because the activated programmable nucleases collaterally cleaves any nucleic acids. If total nucleic acids are in present in large amounts, they may outcompete reporters for the programmable nucleases. The devices and methods disclosed herein are designed to have an excess of reporter to total nucleic acids, such that the detectable signals from cleavage reactions (e.g., DETECTR reactions) are particularly superior. In some embodiments, the reporter can be present in at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold excess of total nucleic acids.

A second significant advantage of the devices and methods disclosed herein is the design of an excess volume comprising the guide nucleic acid, the programmable nuclease, and the reporter, which contacts a smaller volume comprising the sample with the target nucleic acid of interest. The smaller volume comprising the sample can be unlysed sample, lysed sample, or lysed sample which has undergone any combination of reverse transcription, amplification, and in vitro transcription. The presence of various reagents in a crude, non-lysed sample, a lysed sample, or a lysed and amplified sample, such as buffer, magnesium sulfate, salts, the pH, a reducing agent, primers, dNTPs, NTPs, cellular lysates, non-target nucleic acids, primers, or other components, can inhibit the ability of the programmable nuclease to find and cleave the nucleic acid of the reporter. This may be due to nucleic acids that are not the reporter, which outcompete the nucleic acid of the reporter, for the programmable nuclease. Alternatively, various reagents in the sample may simply inhibit the activity of the programmable nuclease. Thus, the devices and methods provided herein for contacting an excess volume comprising the guide nucleic acid, the programmable nuclease, and the reporter to a smaller volume comprising the sample with the target nucleic acid of interest provides for superior detection of the target nucleic acid by ensuring that the programmable nuclease is able to find and cleaves the nucleic acid of the reporter. In some embodiments, the volume comprising the guide nucleic acid, the programmable nuclease, and the reporter (can be referred to as “a second volume”) is 4-fold greater than a volume comprising the sample (can be referred to as “a first volume”). In some embodiments, the volume comprising the guide nucleic acid, the programmable nuclease, and the reporter (can be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold greater than a volume comprising the sample (can be referred to as “a first volume”). In some embodiments, the volume comprising the sample is at least 0.5 ul, at least 1 ul, at least at least 1 μL, at least 2 μL, at least 3 μL, at least 4 μL, at least 5 μL, at least 6 μL, at least 7 μL, at least 8 μL, at least 9 μL, at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 25 μL, at least 30 μL, at least 35 μL, at least 40 μL, at least 45 μL, at least 50 μL, at least 55 μL, at least 60 μL, at least 65 μL, at least 70 μL, at least 75 μL, at least 80 μL, at least 85 μL, at least 90 μL, at least 95 μL, at least 100 μL, from 0.5 μL to 5 μL, from 5 μL to 10 μL, from 10 μL to 15 μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 10 μL to 20 μL, from 5 μL to 20 μL, from 1 μL to 40 μL, from 2 μL to 10 μL, or from 1 μL to 10 μL. In some embodiments, the volume comprising the programmable nuclease, the guide nucleic acid, and the reporter is at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 21 μL, at least 22 μL, at least 23 μL, at least 24 μL, at least 25 μL, at least 26 μL, at least 27 μL, at least 28 μL, at least 29 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, from 10 μL to 15 ul μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 50 μL to 55 μL, from 55 μL to 60 μL, from 60 μL to 65 μL, from 65 μL to 70 μL, from 70 μL to 75 μL, from 75 μL to 80 μL, from 80 μL to 85 μL, from 85 μL to 90 μL, from 90 μL to 95 μL, from 95 μL to 100 μL, from 100 μL to 150 μL, from 150 μL to 200 μL, from 200 μL to 250 μL, from 250 μL to 300 μL, from 300 μL to 350 μL, from 350 μL to 400 μL, from 400 μL to 450 μL, from 450 μL to 500 μL, from 10 μL to 20 μL, from 10 μL to 30 μL, from 25 μL to 35 μL, from 10 μL to 40 μL, from 20 μL to 50 μL, from 18 μL to 28 μL, or from 17 μL to 22 μL.

A reporter may be a hybrid nucleic acid reporter. A hybrid nucleic acid reporter comprises a nucleic acid with at least one deoxyribonucleotide and at least one ribonucleotide. In some embodiments, the nucleic acid of the hybrid nucleic acid reporter can be of any length and can have any mixture of DNAs and RNAs. For example, in some cases, longer stretches of DNA can be interrupted by a few ribonucleotides. Alternatively, longer stretches of RNA can be interrupted by a few deoxyribonucleotides. Alternatively, every other base in the nucleic acid may alternate between ribonucleotides and deoxyribonucleotides. A major advantage of the hybrid nucleic acid reporter is increased stability as compared to a pure RNA nucleic acid reporter. For example, a hybrid nucleic acid reporter can be more stable in solution, lyophilized, or vitrified as compared to a pure DNA or pure RNA reporter.

Additionally, target nucleic acid can be amplified before binding to the crRNA of the CRISPR enzyme. This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C.

Disclosed herein are methods of assaying for a target nucleic acid as described herein wherein a signal is detected. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, a method of assaying for a target nucleic acid in a sample, for example, comprises: a) contacting the sample to a complex comprising a guide nucleic acid comprising a segment that is reverse complementary to a segment of the target nucleic acid (e.g., a nucleic acid from a coronavirus such as SARS-CoV-2) and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

A programmable nuclease can comprise a programmable nuclease capable of being activated when complexed with a guide nucleic acid and target nucleic acid (e.g., a nucleic acid from a coronavirus such as SARS-CoV-2). The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. The signal can be immobilized on a support medium for detection. The signal can be visualized to assess whether a target nucleic acid comprises a modification.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorometric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system is capable of detecting more than one type of target nucleic acid, wherein the system comprises more than one type of guide nucleic acid and more than one type of detector nucleic acid. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the threshold of detection, for a subject method of detecting a single-stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, fom 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the minimum concentration at which a single-stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single-stranded target nucleic acid can be detected in a sample is in a range of from 1 aM to 100 pM. In some cases, the minimum concentration at which a single-stranded target nucleic acid can be detected in a sample is in a range of from 1 fM to 100 pM. In some cases, the minimum concentration at which a single-stranded target nucleic acid can be detected in a sample is in a range of from 10 fM to 100 pM. In some cases, the minimum concentration at which a single-stranded target nucleic acid can be detected in a sample is in a range of from 800 fM to 100 pM. In some cases, the minimum concentration at which a single-stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some cases, the devices, systems, fluidic devices, kits, and methods described herein detect a target single-stranded nucleic acid (e.g., a nucleic acid from a coronavirus such as SARS-CoV-2) in a sample where the sample is contacted with the reagents for a predetermined length of time sufficient for the trans cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the devices, systems, fluidic devices, kits, and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes.

When a guide nucleic acid binds to a target nucleic acid (e.g., a nucleic acid from a coronavirus such as SARS-CoV-2), the programmable nuclease's trans cleavage activity can be initiated, and detector nucleic acids can be cleaved, resulting in the detection of fluorescence. Some methods as described herein can a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The cleaving of the detector nucleic acid using the programmable nuclease may cleave with an efficiency of 50% as measured by a change in a signal that is calorimetric, potentiometric, amperometric, optical (e.g., fluorescent, colorimetric, etc.), or piezo-electric, as non-limiting examples. Some methods as described herein can be a method of detecting a target nucleic acid in a sample comprising contacting the sample comprising the target nucleic acid with a guide nucleic acid targeting a target nucleic acid segment, a programmable nuclease capable of being activated when complexed with the guide nucleic acid and the target nucleic acid segment, a single-stranded detector nucleic acid comprising a detection moiety, wherein the detector nucleic acid is capable of being cleaved by the activated programmable nuclease, thereby generating a first detectable signal, cleaving the single-stranded detector nucleic acid using the programmable nuclease that cleaves as measured by a change in color, and measuring the first detectable signal on the support medium. The cleaving of the single-stranded detector nucleic acid using the programmable nuclease may cleave with an efficiency of 50% as measured by a change in color. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target nucleic acid segment, a programmable nuclease capable of being activated when complexed with the guide nucleic acid and the target nucleic acid segment, and a single-stranded detector nucleic acid comprising a detection moiety, wherein the detector nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample.

In some cases, the methods, reagents, and devices described herein detect a target nucleic acid with a programmable nuclease and a single-stranded detector nucleic acid in a sample where the sample is contacted with the reagents for a predetermined length of time sufficient for trans cleavage of the single-stranded detector nucleic acid. For example, a programmable nuclease is LbuCas13a that detects a target nucleic acid and a single-stranded detector nucleic acid comprises two adjacent uracil nucleotides with a green detectable moiety that is detected upon cleavage. As another example, a programmable nuclease is LbaCas13a that detects a target nucleic acid and a single-stranded detector nucleic acid comprises two adjacent adenine nucleotides with a red detectable moiety that is detected upon cleavage. The target nucleic acid may be a single-stranded nucleic acid (e.g., a single-stranded DNA (ssDNA) or a single-stranded RNA), or the target nucleic acid may be a double-stranded nucleic acid (e.g., a double-stranded DNA (dsDNA) or a double-stranded RNA).

The reagents described herein can also include buffers, which are compatible with the devices, systems, fluidic devices, kits, and methods disclosed herein. These buffers are compatible with the other reagents, samples, and support mediums as described herein for detection of an ailment, such as a disease, including those caused by viruses such as influenza. The methods described herein can also include the use of buffers, which are compatible with the methods disclosed herein. For example, a buffer comprises 20 mM HEPES pH 6.8, 50 mM KCl, 5 mM MgCl₂, and 5% glycerol. In some instances the buffer comprises from 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer can comprise 0 to 25, 0 to 20, 0 to 10, 0 to 5, 5 to 10, 5 to 15, 5 to 20, 5 to 25, 5 to 30% glycerol.

As another example, a buffer comprises 100 mM Imidazole pH 7.5; 250 mM KCl, 25 mM MgCl₂, 50 ug/mL BSA, 0.05% Igepal Ca-630, and 25% Glycerol. In some instances the buffer comprises 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Imidazole pH 7.5. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer, in some instances, comprises 0 to 100, 0 to 75, 0 to 50, 0 to 25, 0 to 20, 0 to 10, 0 to 5, 5 to 50, 5 to 75, 5 to 100, 10 to 20, 10 to 50, 10 to 75, 10 to 100, 25 to 50, 25 to 75 25 to 100, 50 to 75, or 50 to 100 ug/mL BSA. In some instances, the buffer comprises 0 to 1, 0 to 0.5, 0 to 0.25, 0 to 0.01, 0 to 0.05, 0 to 0.025, 0 to 0.01, 0.01 to 0.025, 0.01 to 0.05, 0.01 to 0.1, 0.01 to 0.25, 0.01, to 0.5, 0.01 to 1, 0.025 to 0.05, 0.025 to 0.1, 0.025, to 0.5, 0.025 to 1, 0.05 to 0.1, 0.05 to 0.25, 0.05 to 0.5, 0.05 to 0.75, 0.05 to 1, 0.1 to 0.25, 0.1 to 0.5, or 0.1 to 1% Igepal Ca-630. The buffer can comprise 0 to 25, 0 to 20, 0 to 10, 0 to 5, 5 to 10, 5 to 15, 5 to 20, 5 to 25, 5 to 30% glycerol.

A buffer of the present disclosure may comprise a viral lysis buffer. A viral lysis buffer may lyse a coronavirus capsid in a viral sample (e.g., a sample collected from an individual suspected of having a coronavirus infection), releasing a viral genome. The viral lysis buffer may be compatible with amplification (e.g., RT-LAMP amplification) of a target region of the viral genome. The viral lysis buffer may be compatible with detection (e.g., a DETECTR reaction disclosed herein). A sample may be prepared in a one-step sample preparation method comprising suspending the sample in a viral lysis buffer compatible with amplification, detection (e.g., a DETECTR reaction), or both. A viral lysis buffer compatible with amplification (e.g., RT-LAMP amplification), detection (e.g., DETECTR), or both, may comprise a buffer (e.g., Tris-HCl, phosphate, or HEPES), a reducing agent (e.g., N-Acetyl Cysteine (NAC), Dithiothreitol (DTT), β-mercaptoethanol (BME), or tris(2-carboxyethyl)phosphine (TCEP)), a chelating agent (e.g., EDTA or EGTA), a detergent (e.g., deoxycholate, NP-40 (Ipgal), Triton X-100, or Tween 20), a salt (e.g., ammonium acetate, magnesium acetate, manganese acetate, potassium acetate, sodium acetate, ammonium chloride, potassium chloride, magnesium chloride, manganese chloride, sodium chloride, ammonium sulfate, magnesium sulfate, manganese sulfate, potassium sulfate, or sodium sulfate), or a combination thereof. For example, a viral lysis buffer may comprise a buffer and a reducing agent, or a viral lysis buffer may comprise a buffer and a chelating agent. The viral lysis buffer may be formulated at a low pH. For example, the viral lysis buffer may be formulated at a pH of from about pH 4 to about pH 5. In some embodiments, the viral lysis buffer may be formulated at a pH of from about pH 4 to about pH 9. The viral lysis buffer may further comprise a preservative (e.g., ProClin 150). In some embodiments, the viral lysis buffer may comprise an activator of the amplification reaction. For example, the buffer may comprise primers, dNTPs, or magnesium (e.g., MgSO₄, MgCl₂ or MgOAc), or a combination thereof, to activate the amplification reaction. In some embodiments, an activator (e.g., primers, dNTPs, or magnesium) may be added to the buffer following lysis of the coronavirus to initiate the amplification reaction.

A viral lysis buffer may comprise a pH of about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 6, about 6.5, about 7, about 7.5, about 8, about 8.5, or about 9. In some embodiments, a viral lysis buffer may comprise a pH of from 3.5 to 4.5, from 4 to 5, from 4.5 to 5.5, from 3.5 to 4, from 4 to 4.5, from 4.5 to 5, from 5 to 5.5, from 5 to 6, from 6 to 7, from 7 to 8, or from 8 to 9.

A viral lysis buffer may comprise a magnesium concentration of about 0 mM, about 2 mM, about 4 mM, about 5 mM, about 6 mM, about 8 mM, about 10 mM, about 12 mM, about 13 mM, about 14 mM, about 15 mM, about 20 mM, about 25 mM, about 30 mM, about 35 mM, about 40 mM, about 45 mM, about 50 mM, about 55 mM, or about 60 mM of magnesium (e.g., MgSO₄, MgCl₂ or MgOAc). A viral lysis buffer may comprise a magnesium concentration of from 0 mM to 5 mM, from 5 mM to 10 mM, from 10 mM to 15 mM, from 15 mM to 20 mM, from 20 mM to 25 mM, from 25 mM to 30 mM, from 30 mM to 40 mM, from 40 mM to 50 mM, or from 50 mM to 60 mM of magnesium (e.g., MgSO₄, MgCl₂ or MgOAc). In some embodiments, the magnesium may be added after viral lysis to activate an amplification reaction.

A viral lysis buffer may comprise a reducing agent (e.g., NAC, DTT, BME, or TCEP) at a concentration of about 1 mM, about 2 mM, about 3 mM, about 4 mM, about 5 mM, about 6 mM, about 7 mM, about 8 mM, about 10 mM, about 12 mM, about 15 mM, about 20 mM, about 25 mM, about 30 mM, about 40 mM, about 50 mM, about 60 mM, about 7 mM, about 80 mM, about 90 mM, about 100 mM, or about 120 mM. A viral lysis buffer may comprise a reducing agent (e.g., NAC, DTT, BME, or TCEP) at a concentration of from 1 mM to 5 mM, from 5 mM to 10 mM, from 10 mM to 15 mM, from 15 mM to 20 mM, from 20 mM to 25 mM, from 25 mM to 30 mM, from 30 mM to 40 mM, from 40 mM to 50 mM, from 50 mM to 60 mM, from 60 mM to 70 mM, from 70 mM to 80 mM, or from 80 mM to 90 mM, from 90 mM to 100 mM, or from 100 mM to 120 mM. A viral lysis buffer may comprise a chelator (e.g., EDTA or EGTA) at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 10 mM, about 12 mM, about 15 mM, about 20 mM, about 25 mM, or about 30 mM. A viral lysis buffer may comprise a chelator (e.g., EDTA or EGTA) at a concentration of from 0.1 mM to 0.5 mM, from 0.25 mM to 0.5 mM, from 0.4 mM to 0.6 mM, from 0.5 mM to 1 mM, from 1 mM to 5 mM, from 5 mM to 10 mM, from 10 mM to 15 mM, from 15 mM to 20 mM, from 20 mM to 25 mM, or from 25 mM to 30 mM.

A viral lysis buffer may comprise a salt (e.g., ammonium acetate ((NH₄)₂OAc), magnesium acetate (MgOAc), manganese acetate (MnOAc), potassium acetate (K₂OAc), sodium acetate (Na₂OAc), ammonium chloride (NH₄Cl), potassium chloride (KCl), magnesium chloride (MgCl₂), manganese chloride (MnCl₂), sodium chloride (NaCl), ammonium sulfate ((NH₄)₂SO₄), magnesium sulfate (MgSO₄), manganese sulfate (MnSO₄), potassium sulfate (K₂SO₄), or sodium sulfate (Na₂SO₄)) at a concentration of about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 90 mM, or about 100 mM. A viral lysis buffer may comprise a salt (e.g., (NH₄)₂OAc, MgOAc, MnOAc, K₂OAc, Na₂OAc, NH₄Cl, KCl, MgCl₂, MnCl₂, NaCl, (NH₄)₂SO₄, MgSO₄, MnSO₄, K₂SO₄, or Na₂SO₄) at a concentration of from 1 mM to 5 mM, from 1 mM to 10 mM, from 5 mM to 10 mM, from 10 mM to 15 mM, from 15 mM to 20 mM, from 20 mM to 25 mM, from 25 mM to 30 mM, from 30 mM to 35 mM, from 35 mM to 40 mM, from 40 mM to 45 mM, from 45 mM to 50 mM, from 50 mM to 55 mM, from 55 mM to 60 mM, from 60 mM to 70 mM, from 70 mM to 80 mM, from 80 mM to 90 mM, or from 90 mM to 100 mM.

A viral lysis buffer may comprise a detergent (e.g., deoxycholate, NP-40 (Ipgal), Triton X-100, or Tween 20) at a concentration of about 0.01%, about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.00%, about 1.10%, about 1.20%, about 1.30%, about 1.40%, about 1.50%, about 2.00%, about 2.50%, about 3.00%, about 3.50%, about 4.00%, about 4.50%, or about 5.00%. A viral lysis buffer may comprise a detergent (e.g., deoxycholate, NP-40 (Ipgal), Triton X-100, or Tween 20) at a concentration of from 0.01% to 0.10%, from 0.05% to 0.15%, from 0.10% to 0.20%, from 0.15% to 0.25%, from 0.20% to 0.30%, from 0.25% to 0.35%, from 0.30% to 0.40%, from 0.35% to 0.45%, from 0.40% to 0.50%, from 0.45% to 0.55%, from 0.50% to 0.60%, from 0.55% to 0.65%, from 0.60% to 0.70%, from 0.65% to 0.75%, from 0.70% to 0.80%, from 0.75% to 0.85%, from 0.80% to 0.90%, from 0.85% to 0.95%, from 0.90% to 1.00%, from 0.95% to 1.10%, from 1.00% to 1.20%, from 1.10% to 1.30%, from 1.20% to 1.40%, from 1.30% to 1.50%, from 1.40% to 1.60%, from 1.50% to 2.00%, from 2.00% to 2.50%, from 2.50% to 3.00%, from 3.00% to 3.50%, from 3.50% to 4.00%, from 4.00% to 4.50%, or from 4.50% to 5.00%.

A lysis reaction may be performed at a range of temperatures. In some embodiments, a lysis reaction may be performed at about room temperature. In some embodiments, a lysis reaction may be performed at about 95° C. In some embodiments, a lysis reaction may be performed at from 1° C. to 10° C., from 4° C. to 8° C., from 10° C. to 20° C., from 15° C. to 25° C., from 15° C. to 20° C., from 18° C. to 25° C., from 18° C. to 95° C., from 20° C. to 37° C., from 25° C. to 40° C., from 35° C. to 45° C., from 40° C. to 60° C., from 50° C. to 70° C., from 60° C. to 80° C., from 70° C. to 90° C., from 80° C. to 95° C., or from 90° C. to 99° C. In some embodiments, a lysis reaction may be performed for about 5 minutes, about 15 minutes, or about 30 minutes. In some embodiments, a lysis reaction may be performed for from 2 minutes to 5 minutes, from 3 minutes to 8 minutes, from 5 minutes to 15 minutes, from 10 minutes to 20 minutes, from 15 minutes to 25 minutes, from 20 minutes to 30 minutes, from 25 minutes to 35 minutes, from 30 minutes to 40 minutes, from 35 minutes to 45 minutes, from 40 minutes to 50 minutes, from 45 minutes to 55 minutes, from 50 minutes to 60 minutes, from 55 minutes to 65 minutes, from 60 minutes to 70 minutes, from 65 minutes to 75 minutes, from 70 minutes to 80 minutes, from 75 minutes to 85 minutes, or from 80 minutes to 90 minutes.

A number of detection devices and methods are consistent with methods disclosed herein. For example, any device that can measure or detect a calorimetric, potentiometric, amperometric, optical (e.g., fluorescent, colorometric, etc.), or piezo-electric signal. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorometric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid. Sometimes, the detector nucleic acid is protein-nucleic acid. Often, the protein-nucleic acid is an enzyme-nucleic acid.

The results from the detection region from a completed assay can be detected and analyzed in various ways, for example, by a glucometer. In some cases, the positive control spot and the detection spot in the detection region is visible by eye, and the results can be read by the user. In some cases, the positive control spot and the detection spot in the detection region is visualized by an imaging device or other device depending on the type of signal. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and provide a result. Alternatively or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, cleanup of an environment.

### Disease Detection

Disclosed herein are methods of assaying for a target nucleic acid as described herein that can be used for disease detection. The disease can be a coronavirus. The coronavirus can be SARS-CoV-2, 229E (alpha coronavirus), NL63 (alpha coronavirus), OC43 (beta coronavirus), HKU1 (beta coronavirus), MERS-CoV, or SARS-CoV. In some embodiments, the compositions and methods disclosed herein specifically target and assay for the SARS-CoV-2 coronavirus. For example, a method of assaying for a target nucleic acid (e.g., from a coronavirus) in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The detection of the signal can indicate the presence of the target nucleic acid. Sometimes, the target nucleic acid comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid. Any nucleic acid of the SARS-CoV-2 can be assayed for using the compositions and methods disclosed herein. In some embodiments, the target nucleic acid comprises the N gene or the E gene of coronavirus and can be assayed for using the compositions and methods disclosed herein.

The methods can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Sometimes, a status of a target nucleic acid mutation is used to determine a pathogenicity of a bacteria, virus, or microbe or treatment resistance, such as resistance to antibiotic treatment. Often, a status of a mutation is used to diagnose or identify diseases associated with the mutation of target nucleic acids in the bacteria, virus, or microbe. Often, the mutation is a single nucleotide mutation.

### Detection as a Research Tool, Point-of-Care, or Over-the-Counter

Disclosed herein are methods of assaying for a plurality of target nucleic acid (e.g., a nucleic acid from a coronavirus) as described herein that can be used as a research tool, and can be provided as reagent kits. The coronavirus can be SARS-CoV-2, 229E (alpha coronavirus), NL63 (alpha coronavirus), OC43 (beta coronavirus), HKU1 (beta coronavirus), MERS-CoV, or SARS-CoV. In some embodiments, the compositions and methods disclosed herein specifically target and assay for the SARS-CoV-2 coronavirus. The coronavirus may be a variant of SARS-CoV-2, particularly the United Kingdom (UK) variant known as 20B/501Y.V1, VOC 202012/01, or B.1.1.7 lineage, or the South African variant known as: 20C/501Y.V2 or B.1.351 lineage. Any nucleic acid of the SARS-CoV-2 can be assayed for using the compositions and methods disclosed herein. In some embodiments, the target nucleic acid comprises the N gene or the E gene of coronavirus and can be assayed for using the compositions and methods disclosed herein.

For example, a method of assaying for a plurality of target nucleic acid in a sample comprises contacting the sample to a plurality of complexes comprising a guide nucleic acid comprising a segment that is reverse complementary to a segment of a target nucleic acid of the plurality of target nucleic acids and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The plurality of complexes may comprise programmable nucleases complexes with guide nucleic acids directed to different target nucleic acids. The detection of the signal can indicate the presence of the target nucleic acid. Sometimes, a target nucleic acid of the plurality of target nucleic acids comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

The methods as described herein can be used to identify multiple target nucleic acids. The methods can be used to identify mutation of a target nucleic acid that affects the expression of a gene. A mutation that affects the expression of gene can be a single nucleotide mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation is a single nucleotide mutation.

The reagent kits or research tools can be used to detect any number of target nucleic acids, mutations, or other indications disclosed herein in a laboratory setting. Reagent kits can be provided as reagent packs for open box instrumentation.

In other embodiments, any of the systems, assay formats, Cas reporters, programmable nucleases, or other reagents can be used in a point-of-care (POC) test, which can be carried out at a decentralized location such as a hospital, POL, or clinic. These point-of-care tests can be used to diagnose any of the indications disclosed herein, such as influenza or streptococcal infections, or can be used to measure the presence or absence of a particular mutation in a target nucleic acid (e.g., EGFR). POC tests can be provided as small instruments with a consumable test card, wherein the test card is any of the assay formats (e.g., a lateral flow assay) disclosed herein.

In still other embodiments, any of the systems, assay formats, Cas reporters, programmable nucleases, or other reagents can be used in an over-the-counter (OTC), readerless format, which can be used at remote sites or at home to diagnose a range of indications, such as influenza. These indications can include influenza A, influenza B, streptococcal infections, or CT/NG infections. OTC products can include a consumable test card, wherein the test card is any of the assay formats (e.g., a lateral flow assay) disclosed herein. In an OTC product, the test card can be interpreted visually or using a mobile phone.

### Multiplexing

The devices, systems, fluidic devices, kits, and methods described herein can be multiplexed in a number of ways. These methods of multiplexing are, for example, consistent with methods, reagents, and devices disclosed herein for detection of a target nucleic acid within the sample. A fluidic device may comprise multiple pumps, valves, reservoirs, and chambers for sample preparation, amplification of one or more than one sequences of target nucleic acids within the sample, mixing with a programmable nuclease, and detection of a detectable signal arising from cleavage of detector nucleic acids by the programmable nuclease within the fluidic system itself.

Methods consistent with the present disclosure include a multiplexing method of assaying for a target nucleic acid in a sample. A multiplexing method comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, multiplexing method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Multiplexing can be either spatial multiplexing wherein multiple different target nucleic acids are detected at the same time, but the reactions are spatially separated. Often, the multiple target nucleic acids are detected using the same programmable nuclease, but different guide nucleic acids. The multiple target nucleic acids sometimes are detected using the different programmable nucleases. Sometimes, multiplexing can be single reaction multiplexing wherein multiple different target acids are detected in a single reaction volume. Often, a single population of programmable nucleases is used in single reaction multiplexing. Sometimes, at least two different programmable nucleases are used in single reaction multiplexing. For example, multiplexing can be enabled by immobilization of multiple categories of detector nucleic acids within a fluidic system, to enable detection of multiple target nucleic acids within a single sample.

Furthermore, signals from multiplexing can be quantified. For example, a method of quantification for a disease panel comprises assaying for a plurality of unique target nucleic acids in a plurality of aliquots from a sample, assaying for a control nucleic acid control in a second aliquot of the sample, and quantifying a plurality of signals of the plurality of unique target nucleic acids by measuring signals produced by cleavage of detector nucleic acids compared to the signal produced in the second aliquot. Often the plurality of unique target nucleic acids are from a plurality of viruses in the sample. Sometimes the quantification of a signal of the plurality correlates with a concentration of a unique target nucleic acid of the plurality for the unique target nucleic acid of the plurality that produced the signal of the plurality.

The methods, reagents, and devices described herein can be multiplexed by various configurations of the reagents and the support medium. In some cases, the kit or system is designed to have multiple support mediums encased in a single housing. Sometimes, the multiple support mediums housed in a single housing share a single sample pad. The single sample pad may be connected to the support mediums in various designs such as a branching or a radial formation. Alternatively, each of the multiple support mediums has its own sample pad. In some cases, the kit or system is designed to have a single support medium encased in a housing, where the support medium comprises multiple detection spots for detecting multiple target nucleic acids. Sometimes, the reagents for multiplexed assays comprise multiple guide nucleic acids, multiple programmable nucleases, and multiple single stranded detector nucleic acids, where a combination of one of the guide nucleic acids, one of the programmable nucleases, and one of the single stranded detector nucleic acids detects one target nucleic acid and can provide a detection spot on the detection region. In some cases, the combination of a guide nucleic acid, a programmable nuclease, and a single stranded detector nucleic acid configured to detect one target nucleic acid is mixed with at least one other combination in a single reagent chamber. In some cases, the combination of a guide nucleic acid, a programmable nuclease, and a single stranded detector nucleic acid configured to detect one target nucleic acid is mixed with at least one other combination on a single support medium. When these combinations of reagents are contacted with the sample, the reaction for the multiple target nucleic acids occurs simultaneously in the same medium or reagent chamber. Sometimes, this reacted sample is applied to the multiplexed support medium described herein. In some cases, the methods, reagents, and devices described herein can be multiplexed in a configuration lacking a support medium.

In some cases, the combination of a guide nucleic acid, a programmable nuclease, and a single stranded detector nucleic acid configured to detect one target nucleic acid is provided in its own reagent chamber or its own support medium. In this case, multiple reagent chambers or support mediums are provided in the device, kit, or system, where one reagent chamber is designed to detect one target nucleic acid. In this case, multiple support mediums are used to detect the panel of viral infections, or other diseases of interest.

### Detection of a Target Nucleic Acid in a Fluidic Device

Disclosed herein are various fluidic devices for detection of a target nucleic acid of interest in a biological sample. The target nucleic acid of interest may be from a sample comprising a coronavirus, such as SARS-CoV-2, 229E (alpha coronavirus), NL63 (alpha coronavirus), OC43 (beta coronavirus), HKU1 (beta coronavirus), MERS-CoV, or SARS-CoV. In some embodiments, the target nucleic acid of interest is from the SARS-CoV-2 coronavirus. Any nucleic acid of the SARS-CoV-2 can be a target nucleic acid of interest. In some embodiments, the target nucleic acid of interest comprises the N gene or the E gene of coronavirus. The fluidic devices can be used to monitor the reaction of target nucleic acids in samples with a programmable nuclease, thereby allowing for the detection of said target nucleic acid. All samples and reagents disclosed herein are compatible for use with a fluidic device. Any programmable nuclease, such as any Cas nuclease described herein, are compatible for use with a fluidic device. Support mediums and housing disclosed herein are also compatible for use in conjunction with the fluidic devices. Multiplexing detection, as described throughout the present disclosure, can be carried out within the fluidic devices. Compositions and methods for detection and visualization disclosed herein are also compatible for use within the fluidic systems.

In the below described fluidic systems, any programmable nuclease (e.g., CRISPR-Cas) reaction can be monitored. For example, any programmable nuclease disclosed herein can be used to cleave the reporter molecules to generate a detection signal. In some cases, the programmable nuclease is Cas13. Sometimes the Cas13 is Cas13a, Cas13b, Cas13c, Cas13d, or Cas13e. In some cases, the programmable nuclease is Mad7 or Mad2. In some cases, the programmable nuclease is Cas12. Sometimes the Cas12 is Cas12a, Cas12b, Cas12c, Cas12d, or Cas12e. In some cases, the programmable nuclease is Csm1, Cas9, C2c4, C2c8, C2c5, C2c10, C2c9, or CasZ. Sometimes, the Csm1 is also called smCms1, miCms1, obCms1, or suCms1. Sometimes Cas13a is also called C2c2. Sometimes CasZ is also called Cas14a, Cas14b, Cas14c, Cas14d, Cas14e, Cas14f, Cas14g, Cas14h, Cas14i, Cas14j, or Cas14k. Sometimes, the programmable nuclease is a type V CRISPR-Cas system. In some cases, the programmable nuclease is a type VI CRISPR-Cas system. Sometimes the programmable nuclease is a type III CRISPR-Cas system. In some cases, the programmable nuclease is from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a.

Any microfluidic system or lateral flow assay can be modified to adapt the CRISPR-Cas reactions disclosed herein for assaying and detection of a target nucleic acid from a coronavirus. In some embodiments, signals themselves can be amplified, for example via use of an enzyme such as horse radish peroxidase (HRP). In some embodiments, biotin and avidin reactions, which bind at a 4:1 ratio can be used to immobilize multiple enzymes or secondary signal molecules (e.g., 4 enzymes of secondary signal molecules, each on a biotin) to a single protein (e.g., avidin). In some embodiments, an electrochemical signal may be produced by an electrochemical molecule (e.g., biotin, ferrocene, digoxigenin, or invertase). In some embodiments, the above devices could be couple with an additional concentration step. For example, silica membranes may be used to capture nucleic acids off a column and directly apply the Cas reaction mixture on top of said filter. In some embodiments, the sample chamber of any one of the devices disclosed herein can hold from 20 ul to 1000 ul of volume. In some embodiments, the sample chamber holds from 20 to 500, from 40 to 400, from 30 to 300, from 20 to 200 or from 10 to 100 ul of volume. In preferred embodiments, the sample chamber holds 200 ul of volume. The amplification and detection chambers can hold a lower volume than the sample chamber. For example, the amplification and detection chambers may hold from 1 to 50, 10 to 40, 20 to 30, 10 to 40, 5 to 35, 40 to 50, or 1 to 30 ul of volume. Preferably, the amplification and detection chambers may hold about 200 ul of volume. In some embodiments, an exonuclease is present in the amplification chamber or may be added to the amplification chamber. The exonuclease can clean up single stranded nucleic acids that are not the target. In some embodiments, primers for the target nucleic acid can be phosophorothioated in order to prevent degradation of the target nucleic acid in the presence of the exonuclease. In some embodiments, any of the devices disclosed herein can have a pH balancing well for balancing the pH of a sample. In some embodiments, in each of the above devices, the reporter is present in at least four-fold excess of total nucleic acids (target nucleic acids+non-target nucleic acids). Preferably the reporter is present in at least 10-fold excess of total nucleic acids. In some embodiments, the reporter is present in at least 4-fold, at least 5-fold at least 6-fold, at least 7-fold, at least 8-fold, at least 9-fold, at least 10-fold, at least 15-fold, at least 20-fold, at least 50-fold, at least 100-fold, from 1.5 to 100-fold, from 4 to 80-fold, from 4 to 10-fold, from 5 to 20-fold or from 4 to 15-fold excess of total nucleic acids. In some embodiments, any of the devices disclosed herein can carry out a DETECTR reaction (e.g., a DETECTR reaction to assay for a target nucleic acid from a coronavirus) with a limit of detection of at least 0.1 aM, at least 0.1 nM, at least 1 nM or from 0.1 aM to 1 nM. In some embodiments, the devices disclosed herein can carry out a DETECTR reaction with a positive predictive value of at least 75%, at least 80%, at least 85%, at least 90%, at least 92%, at least 95%, at least 97%, at least 99%, or 100%. In some embodiments, the devices disclosed herein can carry out a DETECTR reaction with a negative predictive value of at least 75%, at least 80%, at least 85%, at least 90%, at least 92%, at least 95%, at least 97%, at least 99%, or 100%. In some embodiments, spatial multiplexing in the above devices is carried out by having at least one, more than one, or every detection chamber in the device comprise a unique guide nucleic acid.

A fluidic device may comprise a plurality of chambers and types of chambers. A fluidic device may comprise a plurality of chambers configured to contain a sample with reagents and in conditions conducive to a particular type of reaction. Such a chamber may be designed to facilitate detection of a reaction or a reaction species (e.g., by having transparent surfaces so that a the contents of the chamber can be monitored by an external fluorimeter, or by having electrodes capable of potentiometric analysis). A fluidic device may comprise an amplification chamber, which can be designed to contain a sample and reagents in conditions (e.g., temperature) suitable for an amplification reaction. A fluidic device may comprise a detection chamber, which may be designed to contain a sample with reagents in conditions suitable for a detection reaction (e.g., a colorimetric reaction or a DETECTR reaction). A fluidic device may also comprise chambers designed to store or transfer reagents. For example, a fluidic device may comprise an amplification reagent chamber designed to hold reagents for an amplification reaction (e.g., LAMP) or a detection reagent chamber designed to hold reagents for a reaction capable of detecting the presence or absence of a species (e.g., a DETECTR reaction). A fluidic device may comprise a chamber configured for multiple purposes (e.g., a chamber may be configured for storing a reagent, containing two types of samples for two separate types of reactions, and facilitating fluorescence detection).

A fluidic device may comprise a sample inlet (the term ‘sample inlet’ is herein used interchangeably with sample inlet port and sample collection port) that leads to an internal space within the fluidic device, such as a chamber or fluidic channel. A sample inlet may lead to a chamber within the fluidic device. A sample inlet may be capable of sealing. In some cases, a sample inlet seals around a second apparatus designed to deliver a sample, thus sealing the sample inlet from the surrounding environment. For example, a sample inlet may be capable of sealing around a swab or syringe. A sample inlet may also be configured to accommodate a cap or other mechanism that covers or seals the A sample inlet may comprise a bendable or breakable component. For example, a sample inlet may comprise a seal that breaks upon sample insertion. In some cases, a seal within a sample inlet releases reagents upon breaking. A sample inlet may comprise multiple chambers or compartments. For example, a sample inlet may comprise an upper compartment and a lower compartment separated by a breakable plastic seal. The seal may break upon sample insertion, releasing contents (e.g., lysis buffer or amplification buffer) from the upper container into the lower container, where it may mix with the sample and elute into a separate compartment (e.g., a sample compartment) within the fluidic device.

In some embodiments, a fluidic device may comprise a sliding valve. A sliding valve may be capable of adopting multiple positions, that connect different channels or compartments in a device. In some cases, a sliding device comprises multiple sets of channels that can simultaneously connect multiple different channels or compartments. For example a device that comprises 10 amplification chambers, 10 reagent chambers, and 1 sample chamber may comprise a sliding valve that can adopt a first position connecting the sample chamber to the 10 amplification chambers through 10 separate channels, and a second position that may separately connect the 10 amplification chambers to the 10 reagent chambers. A sliding valve may be capable of automated control by a device or computer. A sliding valve may comprise a transfer fluidic channel, which can have a first end that is open to a first chamber or fluidic channel and a second end that is blocked when the sliding valve is in a first position, and can have the first end blocked and the second end open to a second chamber or fluidic channel when the sliding valve is in a second position. A sliding valve may be designed to combine the flow from two or more chambers or channels into a single chamber or channel. A sliding valve may be designed to divide the flow from a single chamber or channel into two or more separate chambers or fluidic channels.

A device may comprise a plurality of chambers, fluidic channels and valves. A device may comprise multiple types of chambers, fluidic channels, valves, or any combination thereof. A device may comprise different numbers of chambers, fluidic channels, and valves. For example, a device may comprise one sample chamber, a rotating valve connecting the sample chamber to 10 separate amplification reaction chambers, and two sliding valves controlling flow from the 10 amplification reaction chambers into 30 separate Detection chambers. A rotating valve may connect 2 or more chambers or fluidic channels. A rotating valve may connect 3 or more chambers or fluidic channels. A rotating valve may connect 4 or more chambers or fluidic channels. A rotating valve may connect 5 or more chambers or fluidic channels. A rotating valve may connect 8 or more chambers or fluidic channels. A rotating valve may connect 10 or more chambers or fluidic channels. A rotating valve may connect 15 or more chambers or fluidic channels. A rotating valve may connect 20 or more chambers or fluidic channels.

A fluidic device may comprise a plurality of channels. A fluidic device may comprise a plurality of channels comprising a plurality of dimensions and properties. A fluidic device may comprise two channels with identical lengths. A fluidic device may comprise two channels that provide identical resistance. A fluidic device may comprise two identical channels.

A fluidic device may comprise a millichannel. A millichannel may have a width of between 100 and 200 mm. A millichannel may have a width of between 50 and 100 nm. A millichannel may have a width of between 20 and 50 nm. A millichannel may have a width of between 10 and 20 nm. A millichannel may have a width of between 1 and 10 nm. A fluidic device may comprise a microchannel. A microchannel may have a width of between 800 and 990 μm. A microchannel may have a width of between 600 and 800 m. A microchannel may have a width of between 400 and 600 m. A microchannel may have a width of between 200 and 400 m. A microchannel may have a width of between 100 and 200 m. A microchannel may have a width of between 50 and 100 m. A microchannel may have a width of between 30 and 50 m. A microchannel may have a width of between 20 and 30 μm. A microchannel may have a width of between 10 and 20 m. A microchannel may have a width of between 5 and 10 m. A microchannel may have a width of between 1 and 5 μm. A fluidic device may comprise a nanochannel. A nanochannel may have a width of between 800 and 990 nm. A nanochannel may have a width of between 600 and 800 nm. A nanochannel may have a width of between 400 and 600 nm. A nanochannel may have a width of between 200 and 400 nm. A nanochannel may have a width of between 1 and 200 nm. A channel may have a comparable height and width. A channel may have a greater width than height, or a narrower width than height. A channel may have a width that is 1.1, 1.2, 1.3, 1.4, 1.5, 2, 3, 4, 5, 10, 20, 30, 40, 50, 100, 500, 1000 or more times its height. A channel may have a width that is 0.9, 0.8, 0.7, 0.6, 0.5, 0.25, 0.1, 0.05, 0.01, 0.005, 0.001 times its height. A channel may have a width that is less than 0.001 times its height. A channel may have non-uniform dimensions. A channel may have different dimensions at different points along its length. A channel may divide into 2 or more separate channels. A channel may be straight, or may have bends, curves, turns, angles, or other features of non-linear shapes. A channel may comprise a loop or multiple loops.

A fluidic device may comprise a resistance channel. A resistance channel may be a channel with slow flow rates relative to other channels within the fluidic device. A resistance channel may be a channel with low volumetric flow rates relative to other channels within the fluidic device. A resistance channel may provide greater resistance to sample flow relative to other channels in the fluidic device. A resistance channel may prevent or limit sample backflow. A resistance channel may prevent or limit cross-contamination between multiple samples within a device by limiting turbulence. A resistance channel may contribute to flow stability within a fluidic device. A resistance channel may limit disparities in flow rates between multiple portions of a fluidic device. A resistance channel may stabilize flow rates within a device, and minimize flow variation over time

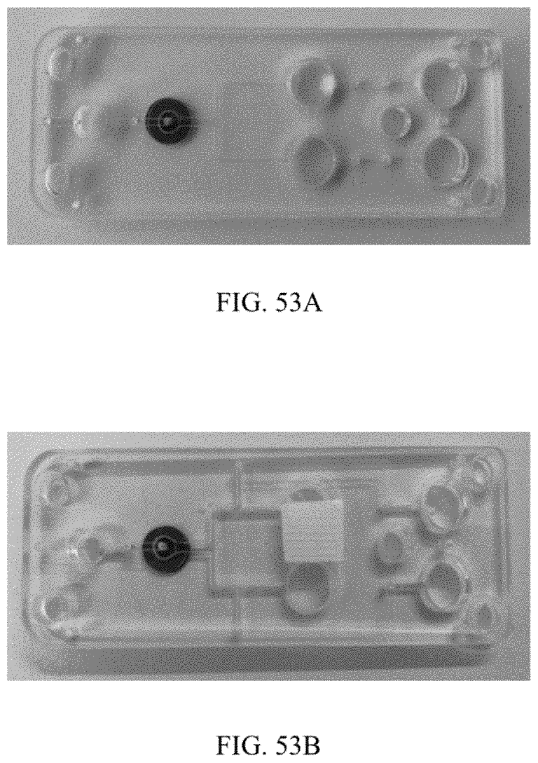

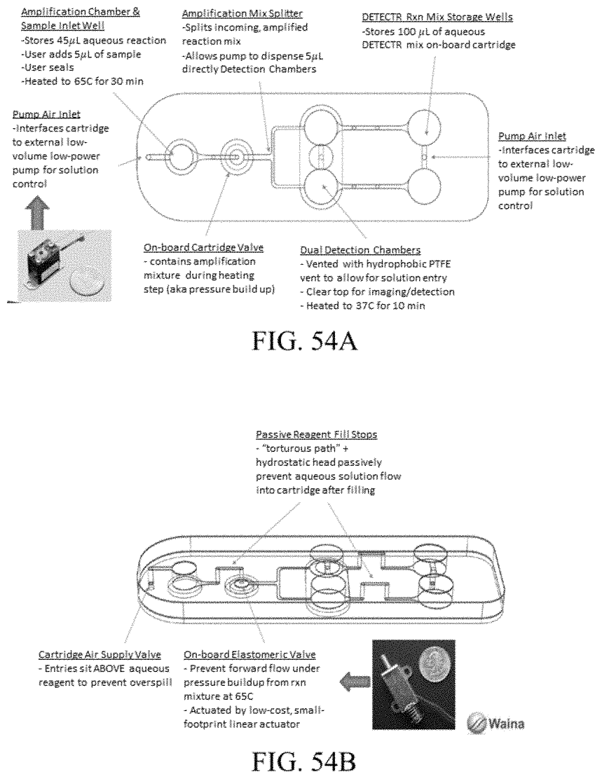

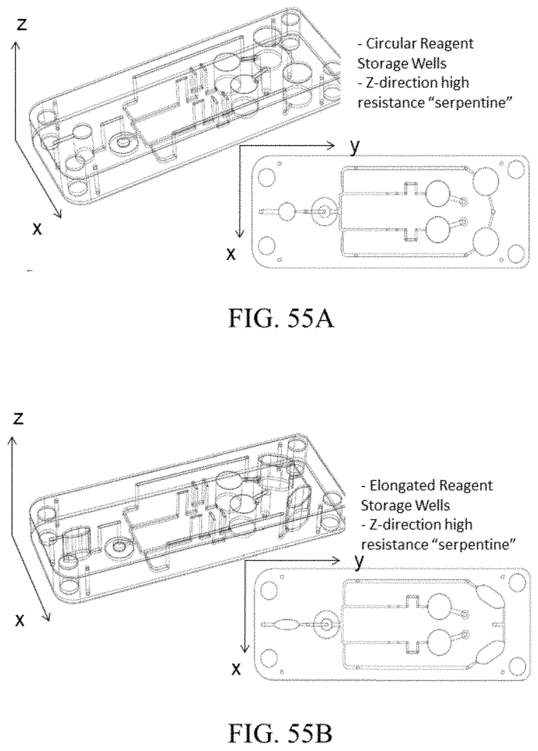

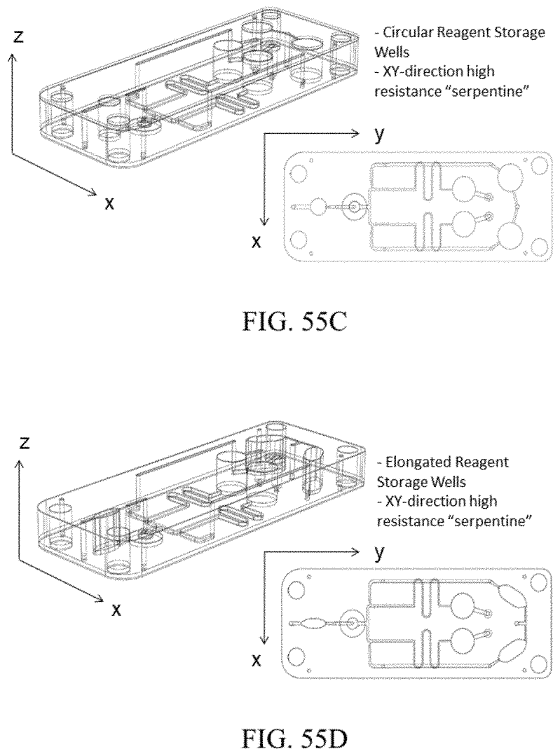

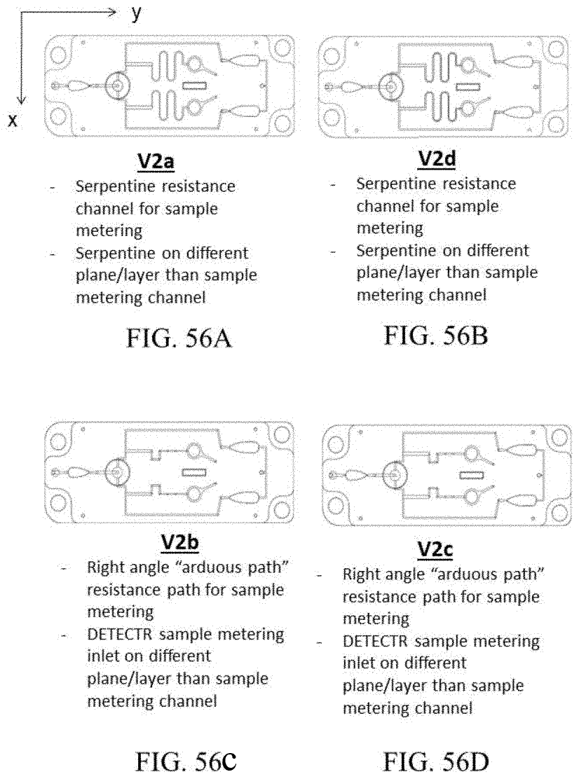

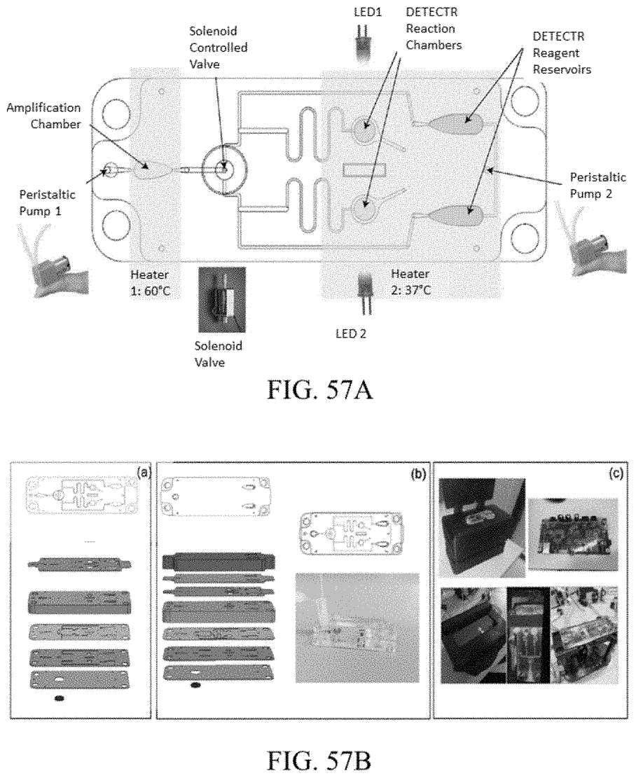

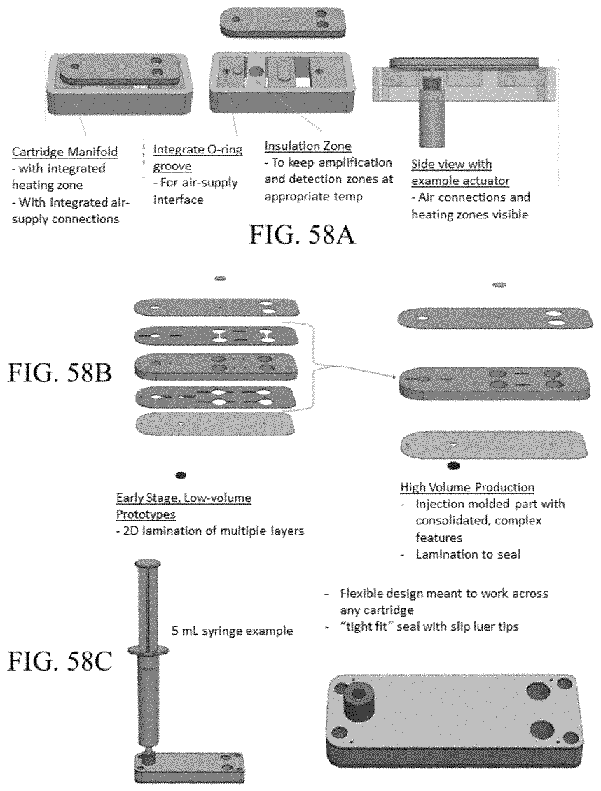

Resistance Channel Devices. In some embodiments, a device of the present disclosure may have resistance channels, sample metering channels, valves for fluid flow or any combination thereof. FIG. 53A, FIG. 53B, FIG. 54A, FIG. 54B, FIG. 55A, FIG. 55B, FIG. 55C, FIG. 55D, FIG. 56A, FIG. 56B, FIG. 56C, and FIG. 56D show examples of said microfluidic cartridges for use in a DETECTR reaction. In some embodiments, a cartridge may comprise an amplification chamber, a valve fluidically connected to the amplification chamber, a detection reaction chamber fluidically connected to the valve, and a detection reagent reservoir fluidically connected to the detection chamber, as shown in FIG. 57A. In some embodiments, a device may further comprise a luer slip adapter, as shown in FIG. 58C. A luer slip adaptor may be used to adapt to a luer lock syringe for sample or reagent delivery into the device. One or more elements (e.g., chambers, channels, valves, or pumps) of a microfluidic device may be fluidically connected to one or more other elements of the microfluidic device. A first element may be fluidically connected to a second element such that fluid may flow between the first element and the second element. A first element may be fluidically connected to a second element through a third element such that fluid may flow from the first element to the second element by passing through the third element. For example, a detection reagent chamber may be fluidically connected to a detection chamber through a resistance channel, as shown in FIG. 57A.

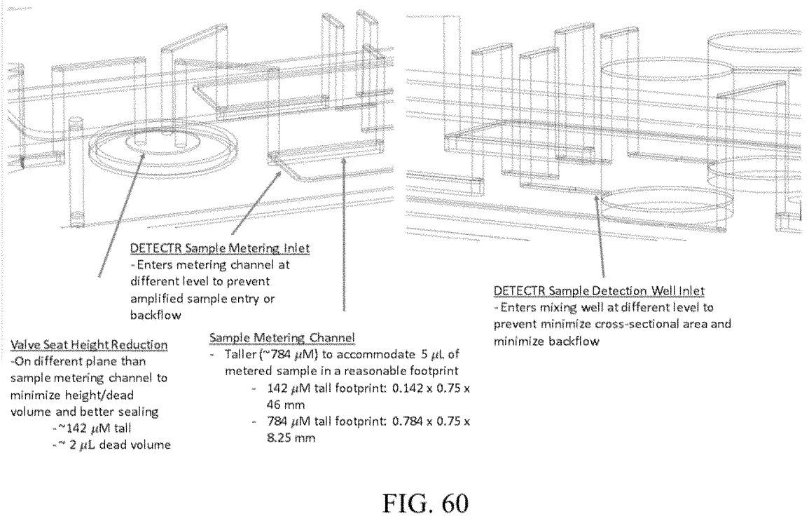

A chamber of the device (e.g., the amplification chamber, the detection chamber, or the detection reagent reservoir) may be fluidically connected to one or more additional chambers by one or more channels. In some embodiments, a channel may be a resistance channel configured to regulate the flow of fluid between a first chamber and a second chamber. A resistance channel may form a non-linear path between the first chamber and the second chamber. It may include features to restrict or confound flow, such as bends, turns, fins, chevrons, herringbones or other microstructures. A resistance channel may have reduced backflow compared to a linear channel of comparable length and width. A resistance channel may function by requiring an increased pressure to pass fluid through the channel compared to a linear channel of comparable length and width. In some embodiments, a resistance channel may result in decreased cross-contamination between two chambers connected by the resistance channel as compared to the cross-contamination between two chambers connected by a linear channel of comparable length and width. A resistance channel may have an angular path, for example as illustrated FIG. 55A, FIG. 55B, FIG. 56C and FIG. 56D. An angular path may comprise one or more angles in the direction of flow of a fluid passing through the channel. In some embodiments, an angular path may comprise a right angle. In some embodiments, an angular path may comprise an angle of about 90°. In some embodiments, an angular path may comprise at least one angle between about 45° and about 135°. In some embodiments, an angular path may comprise at least one angle between about 80° and about 100°. In some embodiments, an angular path may comprise at least one angle between about 85° and about 95°. A resistance channel may have a circuitous or serpentine path, for example as illustrated in FIG. 55C, FIG. 55D, FIG. 56A, and FIG. 56B. A circuitous or serpentine path may comprise one or more bends in the direction of flow of a fluid passing through the channel. In some embodiments, a circuitous or serpentine path may comprise a bend of about 90°. In some embodiments, a circuitous or serpentine path may comprise at least one bend between about 45° and about 135°. In some embodiments, a circuitous or serpentine path may comprise at least one bend between about 80° and about 100°. In some embodiments, a circuitous or serpentine path may comprise at least one bend between about 85° and about 95°. In some embodiments, a resistance channel may be substantially contained within a plane (e.g., the resistance channel may be angular, circuitous, or serpentine in two-dimensions). A two-dimensional resistance channel may be positioned substantially within a single layer of a microfluidic device of the present disclosure. In some embodiments, a resistance channel may be a three-dimensional resistance channel (e.g., the resistance channel may be angular, circuitous, or serpentine in x, y, and z dimensions of a microfluidic device). In some embodiments, a sample input of a resistance channel may be in the same plane (e.g., at the same level in a z direction) as the resistance channel, a chamber connected to the resistance channel, or both. In some embodiments, a sample input of a resistance channel may be in a different plan (e.g., on a different level in a z direction) as the resistance channel, a chamber connected to the resistance channel, or both. Examples of resistance channels are shown in FIG. 60. In some embodiments a resistance channel may have a width of about 300 μm. In some embodiments a resistance channel may have a width of from about 10 μm to about 100 μm, from about 50 μm to about 100 μm, from about 100 μm to about 200 μm, from about 100 m to about 300 μm, from about 100 m to about 400 μm, from about 100 m to about 500 μm, from about 200 m to about 300 μm, from about 200 m to about 400 μm, from about 200 m to about 500 μm, from about 200 μm to about 600 μm, from about 200 m to about 700 μm, from about 200 m to about 800 μm, from about 200 m to about 900 μm, or from about 200 m to about 1000 m.

In some embodiments, a channel may be a sample metering channel. A sample metering channel may form a path between a first chamber and a second chamber and have a channel volume configured to hold a set volume of a fluid to meter the volume of fluid transferred from the first chamber to the second chamber. A sample metering path may form a path between a first chamber and a second chamber and have a channel volume configured to allow to flow from the first channel to the second channel at a desired rate. Metering can also be affected by positive or negative pressure applied to an auxiliary chamber acting as a liquid reagent storage reservoir. This can also be done by storing air in a blister pack for low-cost applications. Examples of sample metering channels are shown in FIG. 60. In some embodiments, a sample input of a sample metering channel may be in the same plane (e.g., at the same level in a z direction) as the sample metering channel, a chamber connected to the sample metering channel, or both. In some embodiments, a sample input of a sample metering channel may be in a different plan (e.g., on a different level in a z direction) as the sample metering channel, a chamber connected to the sample metering channel, or both. The length, width, volume, or combination thereof of a sample metering channel may be designed to pass a desired volume of fluid from a first chamber to a second chamber. The length, width, volume, or combination thereof of a sample metering channel may be designed to pass fluid from a first chamber to a second chamber at a desired rate. In some embodiments, a sample metering channel may have a width of about 300 μm. In some embodiments a sample metering channel may have a width of from about 10 μm to about 100 μm, from about 50 μm to about 100 μm, from about 100 μm to about 200 μm, from about 100 μm to about 300 μm, from about 100 μm to about 400 μm, from about 100 μm to about 500 μm, from about 200 μm to about 300 μm, from about 200 μm to about 400 μm, from about 200 μm to about 500 μm, from about 200 μm to about 600 μm, from about 200 μm to about 700 μm, from about 200 μm to about 800 μm, from about 200 μm to about 900 μm, or from about 200 μm to about 1000 μm. In some embodiments, a first chamber may be connected to a second chamber by a channel comprising a resistance channel and a sample metering channel.

A schematic example of a resistance channel is shown in FIG. 133. The valve seat may have a reduced height of about 142 μm and the valve has a dead volume of about 2 μL. The valve may be positioned on a different plane than the sample metering channel to minimize the seat height and the dead volume and to improve sealing. The DETECTR sample metering inlet may be positioned on a different level than the sample metering channel so that the sample enters the channel at a different height to prevent amplified sample entry or backflow. The sample metering channel may have an increased height of about 784 μm to accommodate 5 μL of metered sample with a footprint of about 0.784 mm×0.75 mm×8.25 mm, as compared to a channel with a height of 142 μm and a footprint of about 0.142 mm×0.75 mm×46 mm. The DETECTR sample detection well inlet may be positioned on a different level than the mixing well so that the DETECTR sample enters the detection well at a different level to reduce the cross sectional area and reduce backflow.

A microfluidic device may comprise one or more reagent ports configured to receive a reagent into the device (e.g., into a chamber of the device). A reagent port may comprise an opening in the wall of a chamber. A reagent port may comprise an opening in the wall of a channel or the end of a channel. A reagent port configured to receive a sample may be a sample inlet port. A reagent (e.g., a buffer, a solution, or a sample) may be introduced into the microfluidic device through a reagent port. The reagent may be introduced manually by a user (e.g., a human user), or the reagent may be introduced automatically by a machine (e.g., by a detection manifold).

A variety of chamber shapes may be utilized in the cartridges of the present disclosure. A chamber may be circular, for example the amplification chambers, detection chambers, and detection reagent reservoirs shown in FIG. 55A and FIG. 55C. A chamber may be elongated, for example the amplification chambers and detection reagent reservoirs shown in FIG. 55B, FIG. 55D, FIG. 56A, FIG. 56B, FIG. 56C, and FIG. 56D.

A valve may be configured to prevent, regulate, or allow fluid flow from a first chamber to one or more additional chambers. In some embodiments, a valve may rotate from a first position to a second position to prevent, allow, or alter a fluid flow path. In some embodiments, a valve may slide from a first position to a second position to prevent, allow, or alter a fluid flow path. In some embodiments, a valve may open or close based on pressure applied to the valve. In some embodiments, a valve may be an elastomeric valve. The valve can be active (mechanical, non-mechanical, or externally actuated) or passive (mechanical or non-mechanical). A valve may be controlled electronically. For example, a valve may be controlled using a solenoid. In some embodiments, a valve may be controlled manually. Other mechanisms of control may be: magnetic, electric, piezoelectric, thermal, bistable, electrochemical, phase change, rheological, pneumatic, check valving or capillarity. In some embodiment, a valve may be disposable. For example, a valve may be removed from a microfluidic device and replaced with a new valve to prevent contamination when reusing a microfluidic device.

The cartridge may be configured to connect to a first pump to pump fluid from the amplification chamber to the detection chamber and to a second pump to pump fluid from the detection reagent reservoir to the detection chamber. A variety of pumps known in the art are functional to move fluid from a first chamber to a second chamber and may be used with a cartridge of the present disclosure. In some embodiments, a cartridge may be used with a peristaltic pump, a pneumatic pump, a hydraulic pump, or a syringe pump.

An example of a microfluidic cartridge is shown in FIG. 54A and FIG. 54B. As shown in FIG. 54A, the cartridge may contain an amplification chamber and sample inlet well capable of storing about 45 μL of aqueous reaction mix to which a user adds about 5 μL of sample. The amplification chamber may be sealed. A pump air inlet interfaces the cartridge to an external low-volume low-power pump for solution control. The on-board cartridge valve may be configured to contain amplification mixture during the heating step and during pressure build-up. The cartridge ma contain an amplification mix splitter to split the incoming amplification reaction mix and allows a pump to dispense about 5 μL directly to the detection chambers. Dual detection chambers can be vented with hydrophobic PTFE vent to allow solution entry, have a clear top for imaging and detection, and may be heated to 37° C. for 10 minutes during a reaction. In some embodiments, a detection chamber may be sized such that an amplified sample mixture fills the detection chamber when combined with the detection reagents from the detection reagent storage chamber. DETECTR reaction mix storage wells, also referred to as a detection reagent storage chambers, can store about 100 μL of aqueous DETECTR mix on-board the cartridge. The pump air inlet interfaces the cartridge to an external low-volume low-power pump for solution control. As shown in FIG. 54B, the cartridge may contain a cartridge air supply valves, and entries sit above aqueous reagent to prevent overspill. Passive reagent fill stops form a torturous path and have hydrostatic head to passively prevent aqueous solution flow into cartridge after filling. The on-board elastomeric valve prevents forward flow under pressure build-up from the reaction mixture heated to 65° C. and is actuated by a low-cost, small-footprint linear actuator.

In some embodiments, a device may comprise a multi-layered, laminated cartridge patterned with laser embossing, and hardware with integrated electronics, optics and mechanics, as shown in FIG. 57B. A multi-layered device may be manufactured by two-dimensional lamination, as shown in FIG. 58B (left). In some embodiments, a device may be injection molded. An injection molded device may be laminated to seal the device, as shown in FIG. 58B (right). Injection molding may be used for high volume production of a microfluidic device of the present disclosure.

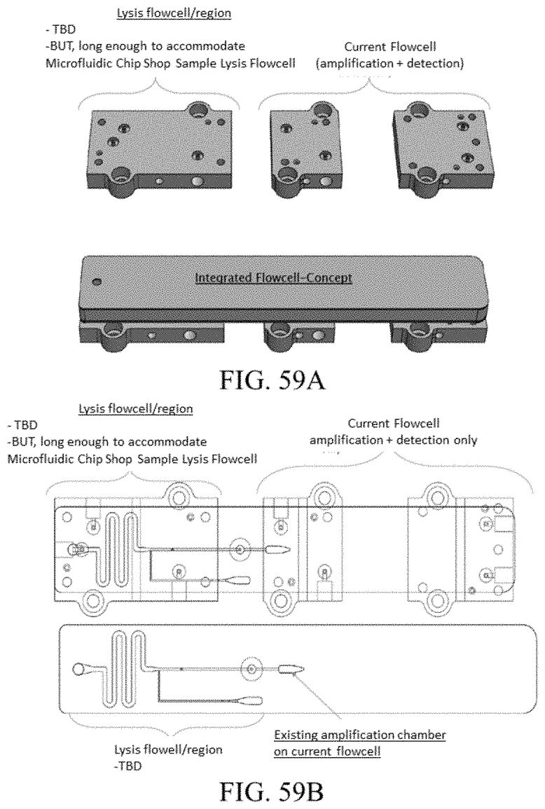

Detection Manifolds. A detection manifold may be used to perform and detect a DETECTR assay of the present disclosure in a device of the present disclosure. A detection manifold may also be referred to herein as a cartridge manifold or a heating manifold. A detection manifold may be configured to facilitate or detect a DETECTR reaction performed in a microfluidic device of the present disclosure. In some embodiments, a detection manifold may comprise one or more heating zones to heat one or more regions of a microfluidic device. In some embodiments, a detection manifold may comprise a first heating zone to heat a first region of a microfluidic device in which an amplification reaction is performed. For example, the first heater may heat the first region of the microfluidic device to about 60° C. In some embodiments, a detection manifold may comprise a second heating zone to heat a second region of a microfluidic device in which a detection reaction is performed. For example, the second heater may heat the second region of the microfluidic device to about 37° C. In some embodiments, a detection manifold may comprise a third heating zone to heat a third region of a microfluidic device in which a lysis reaction is performed. For example, the third heater may heat the third region of the microfluidic device to about 95° C. An example of a detection manifold comprising two insulated heating zones for use with a microfluidic cartridge is shown in FIG. 58A. In some embodiments, a detection manifold may comprise a heating zone configured to heat a lysis region of a microfluidic device of the presence disclosure. An example of a detection manifold comprising a lysis heating zone, an amplification heating zone, and a detection heating zone is shown in FIG. 59A and FIG. 59B. The detection manifold may be configured to be compatible with a microfluidic device comprising a lysis chamber, an amplification chamber, and a detection chamber.

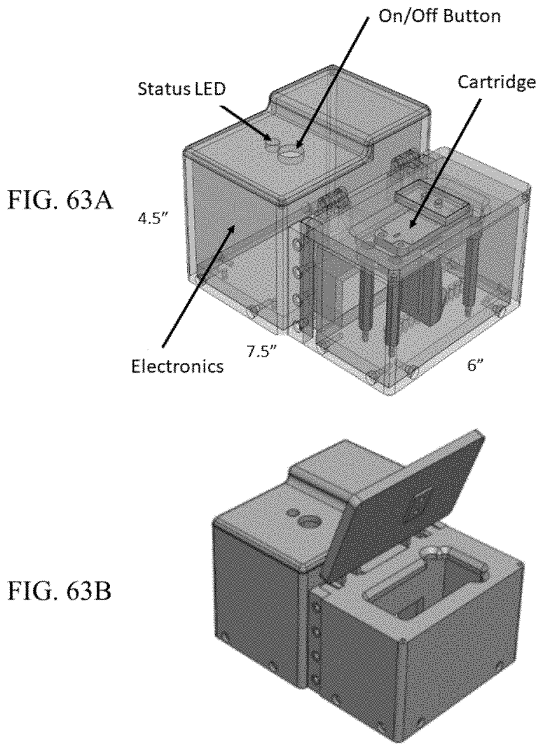

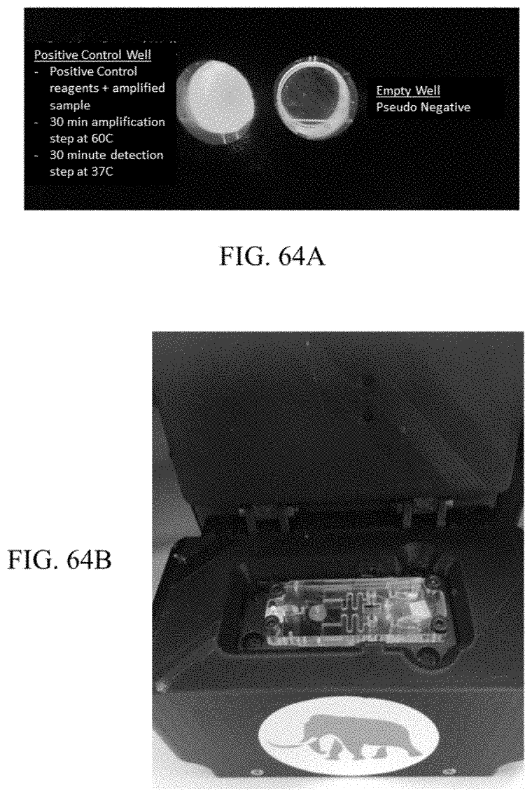

In some embodiments, a detection manifold may comprise an illumination source configured to illuminate a detection chamber of a microfluidic device. The illumination source may be configured to emit a narrow spectrum illumination (e.g., an LED) or the illumination may be configured to emit a broad-spectrum illumination (e.g., an arc lamp). The detection manifold may further comprise one or more filters or gratings to filter for a desired illumination wavelength. In some embodiments, the illumination source may be configured to illuminate a detection chamber (e.g., a chamber comprising a DETECTR reaction) through atop surface of a microfluidic device. In some embodiments, the illumination source may be configured to illuminate a detection chamber through a side surface of a microfluidic device. In some embodiments, the illumination source may be configured to illuminate a detection chamber through a bottom surface of a microfluidic device. In some embodiments, the detection manifold may comprise a sensor for detecting a signal produced by a DETECTR reaction. The signal may be a fluorescent signal. For example, the detection manifold may comprise a camera (e.g., charge-coupled device (CCD), complementary metal-oxide-semiconductor (CMOS)) or a photodiode. A schematic example of a detection manifold is shown in FIG. 63A and FIG. 63B. An example of a detection illuminated in a detection manifold is shown in FIG. 64A.

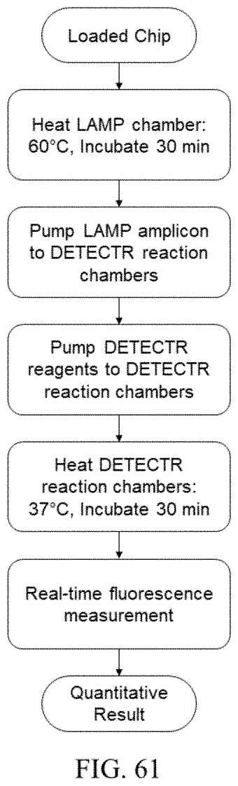

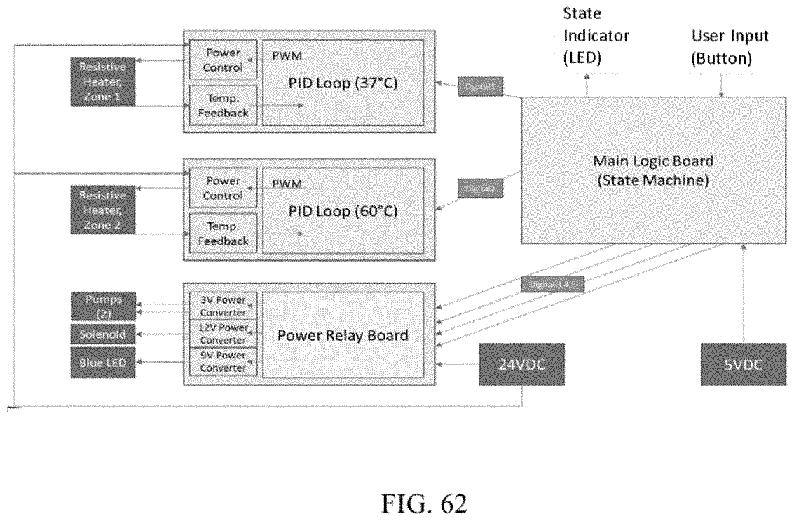

A detection manifold may comprise electronics configured to control one or more of a temperature, a pump, a valve, an illumination source, or a sensor. In some embodiments, the electronics may be controlled autonomously using a program. For example, the electronics may be autonomously controlled to implement a workflow of the present disclosure (e.g., the workflow provided in FIG. 61. A schematic example of an electronic layout is provided in FIG. 62. The electronics may control one or more heaters using one or more of a power control, a temperature feedback, or a PID loop. One or more of a pump, a valve (e.g., a solenoid-controlled valve), or an LED (e.g., a blue LED) may be controlled by one or more of a power converter (e.g., a 3V, 12V, or 9V power converter) or a power relay board. A logic board may be used to control one or more elements of the detection manifold. A detection manifold may comprise one or more indicator lights to indicate a status of one or more elements (e.g., an LED, a heater, a pump, or a valve). The devices described in this section may be combined with any other features disclosed herein (e.g., pneumatic valves, components that operate via use of sliding valves, or any other general feature of devices disclosed herein).

In some cases, detection or visualization may comprise the production of light by a diode. In some cases, a diode may produce visible light. In some cases, a diode may produce infrared light. In some cases, a diode may produce ultraviolet light. In some cases, a diode may be capable of producing different wavelengths or spectra of light. A diode may produce light over a broad or narrow spectrum. A diode may produce white light covering a large portion of the visible spectrum. A diode may produce a specific wavelength of light (e.g., a roughly Gaussian or Lorentzian wavelength vs intensity profile centered around a particular wavelength). In some cases, the bandwidth of light produced by a diode may be defined as the full width at half maximum intensity of a Gaussian-like or Lorentzian-like band. Some diodes produce light with narrow emission bandwidths. A diode may produce light with less than a 1 nm bandwidth. A diode may produce light with less than a 5 nm bandwidth. A diode may produce light with less than a 10 nm bandwidth. A diode may produce light with less than a 20 nm bandwidth. A diode may produce light with less than a 30 nm bandwidth. A diode may produce light with less than a 50 nm bandwidth. A diode may produce light with less than a 100 nm bandwidth.

In some cases, detection or visualization may comprise light detection by a diode. The current produced by a diode may be used to determine characteristics of light absorbed, including polarization, wavelength, intensity, direction traveled, point of origin, or any combination thereof.

In some cases, a diode array may be used to excite and detect fluorescence from a sample. In some cases, a device may comprise a light producing diode and detector diode positioned to illuminate and detect light from a particular portion of a sample. In some cases, a device may comprise a light producing diode and detector diode positioned to illuminate and detect light from a particular sample compartment or chamber.

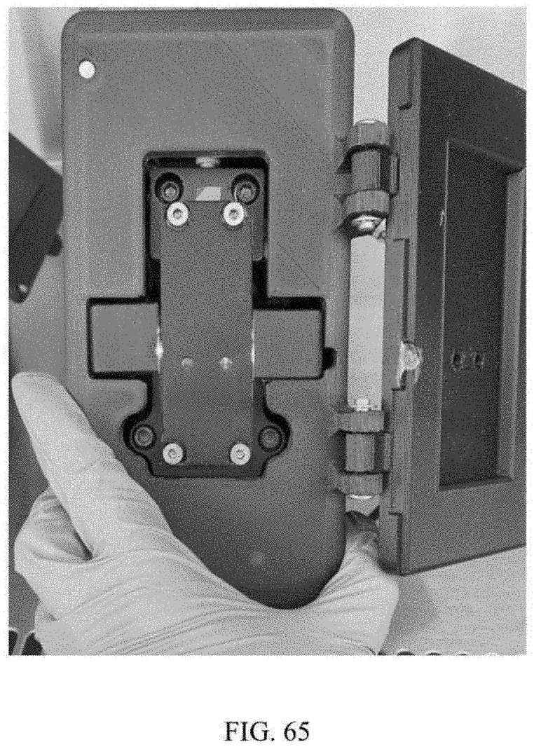

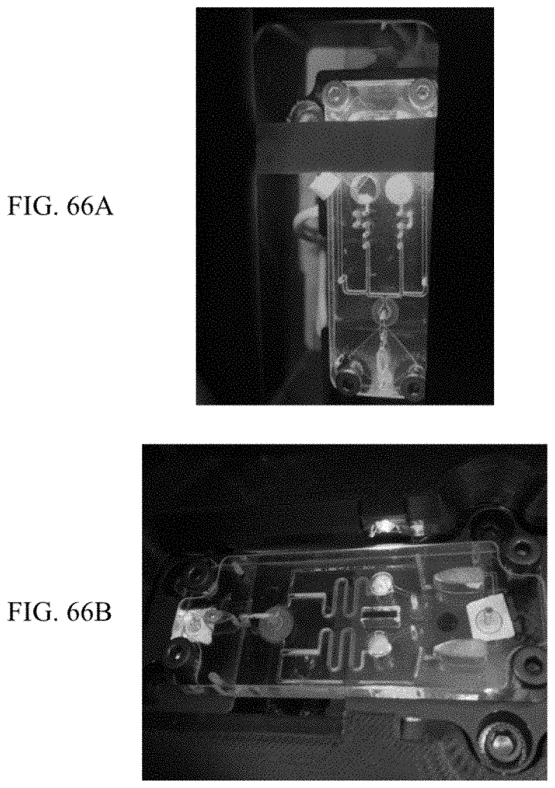

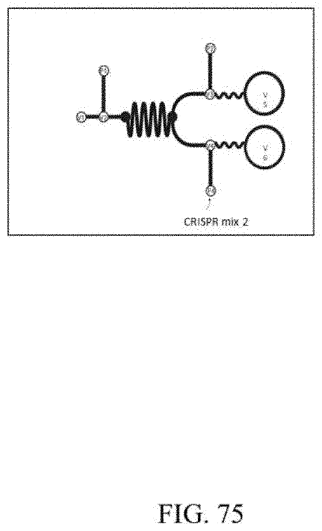

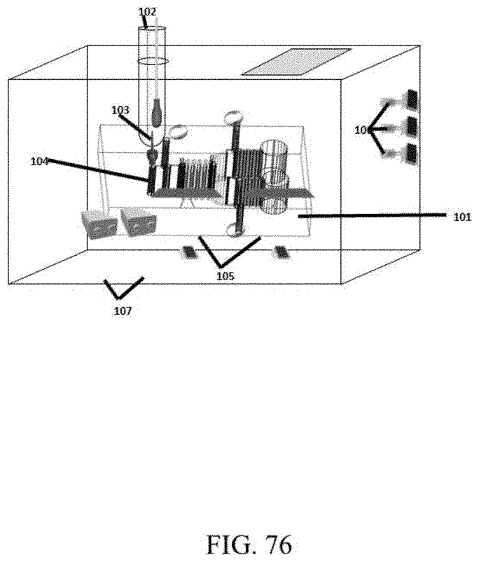

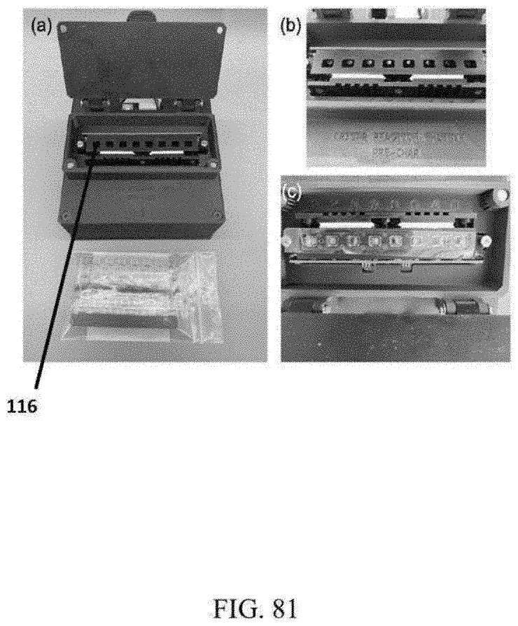

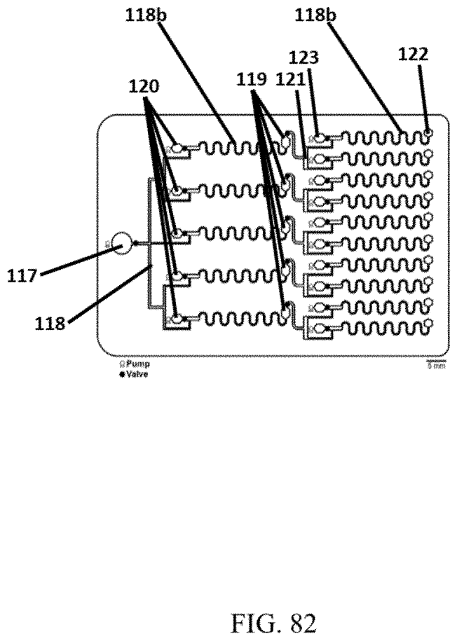

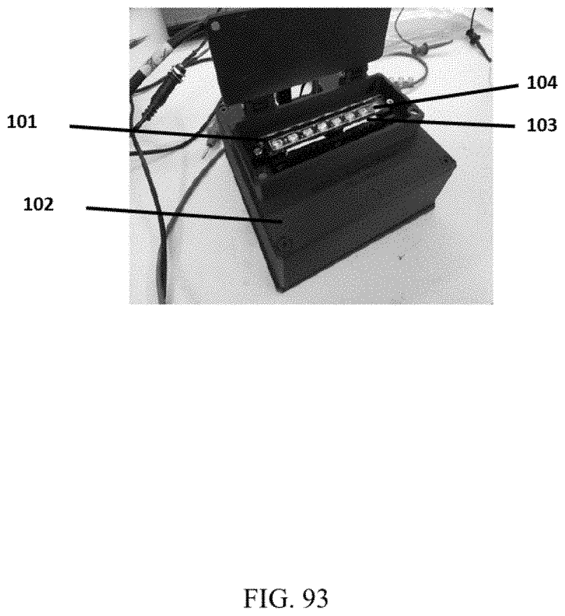

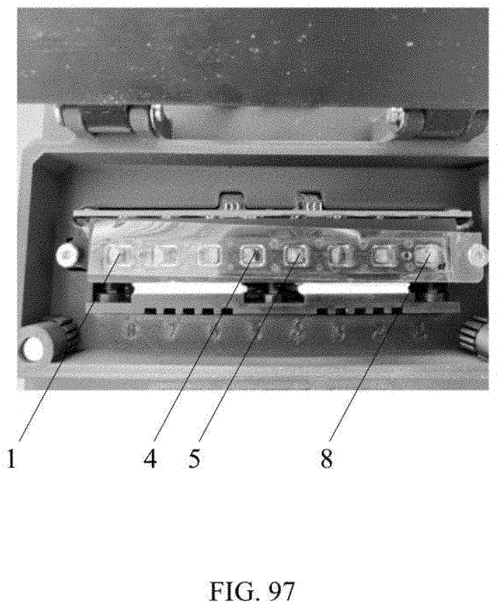

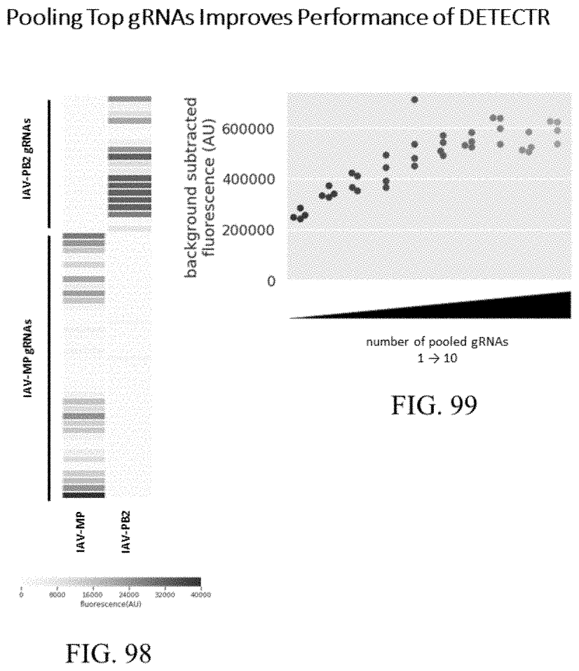

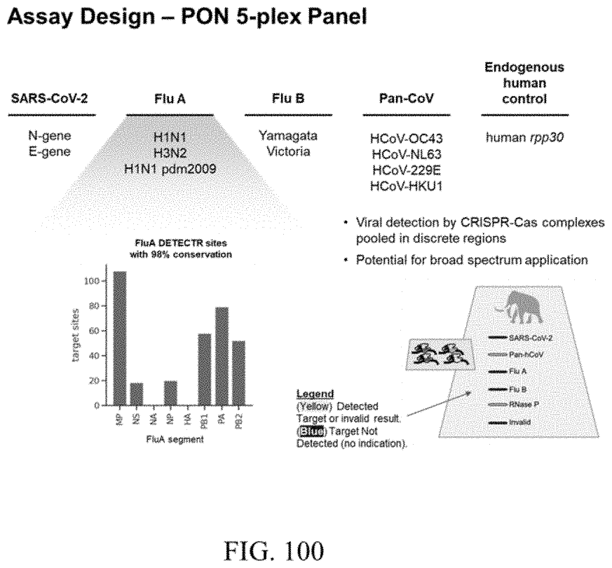

Workflows. A DETECTR reaction may be performed in a microfluidic device using many different workflows. In some embodiments, a workflow for measuring a buccal swab sample may comprise swabbing a cheek, adding the swab to a lysis solution, incubating the swab to lyse the sample, combining the lysed sample with reagents for amplification of a target nucleic acid, combining the amplified sample with DETCTR reagents, and incubating the sample to detect the target nucleic acid. In some embodiments, one or more of lysis, amplification, and detection may be performed in a microfluidic device (e.g., a microfluidic cartridge illustrated in FIG. 53A-B, FIG. 54A-B, FIG. 55A-D, FIG. 56A-D, FIG. 57A, FIG. 60, FIG. 75, FIG. 76, or FIG. 82-FIG. 92. In some embodiments, the workflow may comprise measuring a detectable signal indicative of the presence or absence of a target nucleic acid using a detection manifold (e.g., a detection manifold illustrated in FIG. 136A-B, FIG. 64B, FIG. 65, FIG. 81, FIG. 93, or FIG. 97).

An example of a workflow for detecting a target nucleic acid is provided in FIG. 61. The cartridge may be loaded with a sample and reaction solutions. The amplification chamber may be heated to 60° C. and the sample may incubated in the amplification chamber for 30 minutes. The amplified sample may be pumped to the DETECTR reaction chambers, and the DETECTR reagents may be pumped to the DETECTR reaction chambers. The DETECTR reaction chambers may be heated to 37° C. and the sample may be incubated for 30 minutes. The fluorescence in the DETECTR reaction chambers may be measured in real time to produce a quantitative result.

An example of a workflow for detecting a target nucleic acid (e.g., a viral target nucleic acid) may comprise swabbing a cheek of a subject. The swab may be added to about 200 μL of a low-pH solution. In some embodiments, the swab may displace the solution so that the total volume is about 220 μL. The swab may be incubated in the low-pH solution for about a minute. In some embodiments, cells or viral capsids present on the swab may be lysed in the low-pH solution. A portion of the sample (5 μL) may be combined with about 45 μL of an amplification solution in an amplification chamber. The total volume within the chamber may be about 50 μL. The sample may be incubated in the amplification chamber for up to about 30 minutes at a temperature of from about 50° C. to about 65° C. to amplify the target nucleic acid the sample. In some embodiments, two aliquots of about 5 μL each of the amplified sample may be directed to two detection chambers where they are combined with about 95 μL each of a DETECTR reaction mix. The amplified sample may be incubated with the DETECTR reaction mix for up to about 10 minutes at about 37° C. in each of two detection chambers to detect the presence or absence of the target nucleic acid.

In some embodiments, a workflow for a DETECTR reaction performed in a microfluidic device may be implemented by a user. A user may collect a sample from a subject (e.g., a buccal swab or a nasal swab), place the sample in a lysis buffer, add the lysed sample to a microfluidic cartridge of the present disclosure, and insert the cartridge in a detection manifold of the present disclosure. In some embodiments, a user may add an unlysed sample to the microfluidic cartridge. In some embodiments, a workflow for a DETECTR reaction may be implemented in a microfluidic cartridge of the present disclosure. A microfluidic cartridge may comprise one or more reagents in one or more chambers to facilitate one or more of lysis, amplification, or detection of a target nucleic acid in a sample. In some embodiments, a workflow for a DETECTR reaction performed in a microfluidic device may be facilitated by a detection manifold. A detection manifold may provide one or more of heating control for an amplification reaction, a detection reaction, or both, solution movement control (e.g., pump control or valve control), illumination, or detection.

In some embodiments, a workflow for a DETECTR performed a microfluidic cartridge and facilitated by a user and a detection manifold may comprise steps of: 1) user loads sample into cartridge comprising one or more reagents, 2) user inserts cartridge into a detection manifold and presses a start button, 3) manifold energizes a solenoid to close a valve between a amplification chamber and a detection chamber, 4) manifold indicator LED turns on, 5) manifold turns on first heater to heat a first heating zone to 60° C. and second heater to heat a second heating zone to 37° C., 5) incubate sample in amplification chamber for 30 minutes in first heating zone to amplify sample, 6) manifold turns off first heater, 7) manifold de-energizes solenoid to open valve, 8) manifold turns on a first pump for 15 seconds to pump the amplified sample to the detection chamber, 9) manifold turns off first pump, 10) manifold turns on a second pump for 15 seconds to pump detection reagents from a detection reagent storage chamber to the detection chamber, 11) manifold turns off second pump, 12) incubate amplified sample and detection reagents in detection chamber for 30 minutes in second heating zone to perform detection reaction, 13) manifold indicator LED turns off, 14) manifold turns on illumination source and measures detectable signal produced by detection reaction.

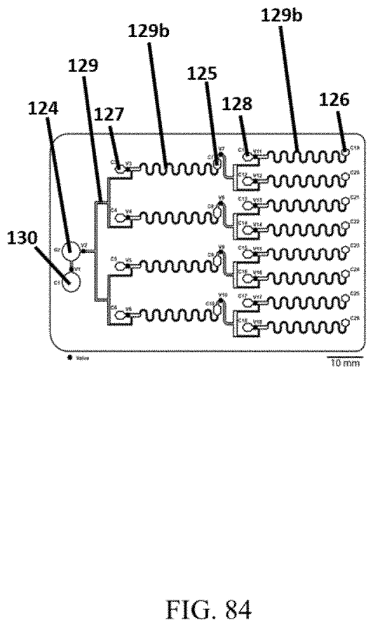

An example of a workflow that may be performed in a microfluidic device, for example the microfluidic device shown in FIG. 84, and facilitated by a detection manifold, for example the detection manifold shown in FIG. 93, may comprise the following steps: 1) Add a swab containing a sample to chamber C2 while valves V1-V18 are closed, heater 1 is off, and heater 2 is off; 2) snap off the end of the swab and close the lid of the device; 3) suspend swab in lysis solution by opening valve V1 to facilitate flow of lysis solution from chamber C1 to chamber C2; 4) meter about 20 μL of lysate from chamber C2 to each of chambers C7-C10 by opening valve V2 and mix with contents from chambers C3-C6 by opening valves V3-V6; 5) close all valves and turn on heater 1 to incubate the samples in chambers C7-C10 at 60° C. to amplify; 6) turn off heater 1, meter about 10 μL of amplicon into each of chambers C19-C26 from chambers C7-C10 (2×10 μL from each chamber), and combine with the contents from each of chambers C11-C18 by opening valves V7-V18; 7) close all valves and turn on heater 2 to incubate the sample in chambers C19-C26 at 37° C. to perform CRISPR detection reaction; 8) detect the samples in chambers C19-C26 by illuminating at 470 nm and detecting at 520 nm during the incubation of step 7.

In some embodiments, a workflow performed in microfluidic device may comprise partitioning a sample into two or more chambers. A device may be configured to partition a sample into a plurality of portions. A device may be configured to transfer two portions of a partitioned sample into separate fluidic channels or chambers. A device may be configured to transfer a plurality of portions of a sample into a plurality of different fluidic channels or chambers. A device may be configured to perform reactions on individual portions of a partitioned sample. A device may be configured to partition a sample into 2 portions. A device may be configured to partition a sample into 3 portions. A device may be configured to partition a sample into 4 portions. A device may be configured to partition a sample into 5 portions. A device may be configured to partition a sample into 6 portions. A device may be configured to partition a sample into 7 portions. A device may be configured to partition a sample into 8 portions. A device may be configured to partition a sample into 9 portions. A device may be configured to partition a sample into 10 portions. A device may be configured to partition a sample into 12 portions. A device may be configured to partition a sample into 15 portions. A device may be configured to divide a sample into at least 20 portions. A device may be configured to partition a sample into at least 50 portions. A device may be configured to partition a sample into 100 portions. A device may be configured to partition a sample into 500 portions.

A device may be configured to perform a first reaction on a first portion of a sample and a second reaction on a second portion of a partitioned sample. A device may be configured to perform a different reaction on each portion of a partitioned sample. A device may be configured to perform sequential reactions on a sample or a portion of a sample. A device may be configured to perform a first reaction in a first chamber and a second reaction in a second chamber on a sample or portion of a sample.

A device may be configured to mix a sample with reagents. In some cases, a device mixes a sample with reagents by flowing the sample and reagents back and forth between a plurality of compartments. In some cases, a device mixes a sample with reagents by cascading the sample and reagents into a single compartment (e.g., by flowing both the sample and reagents into the compartment from above). In some cases, the mixing method performed by the device minimizes the formation of bubbles. In some cases, the mixing method performed by the device minimizes the sample loss or damage (e.g., protein precipitation).

A device may be configured to perform a plurality of reactions on a plurality of portions of a sample. In some cases, a device comprises a plurality of chambers each comprising reagents. In some cases, two chambers from among the plurality of reagent comprising chambers comprise different reagents. In some cases, a first portion and a second portion of a sample may be subjected to different reactions. In some cases, a first portion and a second portion of a sample may be subjected to the same reactions in the presence of different reporter molecules. In some cases, a first portion and a second portion of a sample may be subjected to the same detection method. In some cases, a first portion and a second portion of a sample may be subjected to different detection methods. In some cases, a plurality of portions of a sample may be detected separately (e.g., by a diode array that excites and detects fluorescence from each portion of a sample individually). In some cases, a plurality of portions of a sample may be detected simultaneously. For example, a device may partition a single sample into 4 portions, perform different amplification reactions on each portion, partition the products of each amplification reaction into two portions, perform different DETECTR reactions on each portion, and individually measure the progress of each DETECTR reaction.

A device may be configured to partition a small quantity of sample for a large number of different reactions or sequences of reactions. In some cases, a device may partition less than 1 ml of sample for a plurality of different reactions or sequences of reactions. In some cases, a device may partition less than 800 μl of sample for a plurality of different reactions or sequences of reactions. In some cases, a device may partition less than 600 μl of sample for a plurality of different reactions or sequences of reactions. In some cases, a device may partition less than 400 μl of sample for a plurality of different reactions or sequences of reactions. In some cases, a device may partition less than 200 μl of sample for a plurality of different reactions or sequences of reactions. In some cases, a device may partition less than 100 μl of sample for a plurality of different reactions or sequences of reactions. In some cases, a device may partition less than 50 μl of sample for a plurality of different reactions or sequences of reactions. In some cases, a device may partition less than 1 mg of sample for a plurality of different reactions or sequences of reactions. In some cases, a device may partition less than 800 μg of sample for a plurality of different reactions or sequences of reactions. In some cases, a device may partition less than 600 μg of sample for a plurality of different reactions or sequences of reactions. In some cases, a device may partition less than 400 μg of sample for a plurality of different reactions or sequences of reactions. In some cases, a device may partition less than 200 μg of sample for a plurality of different reactions or sequences of reactions. In some cases, a device may partition less than 100 μg of sample for a plurality of different reactions or sequences of reactions. In some cases, a device may partition less than 50 μg of sample for a plurality of different reactions or sequences of reactions. In some cases, a device may partition less than 20 μg of sample for a plurality of different reactions or sequences of reactions. In some cases, a device may partition less than 10 μg of sample for a plurality of different reactions or sequences of reactions. In some cases, a device may partition less than 1 μg of sample for a plurality of different reactions or sequences of reactions. In some cases, a device may partition less than 800 ng of sample for a plurality of different reactions or sequences of reactions. In some cases, a device may partition less than 600 ng of sample for a plurality of different reactions or sequences of reactions. In some cases, a device may partition less than 400 ng of sample for a plurality of different reactions or sequences of reactions. In some cases, a device may partition less than 200 ng of sample for a plurality of different reactions or sequences of reactions. In some cases, a device may partition less than 100 ng of sample for a plurality of different reactions or sequences of reactions. In some cases, a device may partition less than 50 ng of sample for a plurality of different reactions or sequences of reactions. In some cases, the sample may comprise nucleic acid. In some cases, the sample may comprise cells. In some cases, the sample may comprise proteins. In some cases, the plurality of different reactions or sequences of reactions may comprise 2 or more different reactions or sequences of reactions. In some cases, the plurality of different reactions or sequences of reactions may comprise 3 or more different reactions or sequences of reactions. In some cases, the plurality of different reactions or sequences of reactions may comprise 4 or more different reactions or sequences of reactions. In some cases, the plurality of different reactions or sequences of reactions may comprise 5 or more different reactions or sequences of reactions. In some cases, the plurality of different reactions or sequences of reactions may comprise 10 or more different reactions or sequences of reactions. In some cases, the plurality of different reactions or sequences of reactions may comprise 20 or more different reactions or sequences of reactions. In some cases, the plurality of different reactions or sequences of reactions may comprise 50 or more different reactions or sequences of reactions. In some cases, the plurality of different reactions or sequences of reactions may comprise 100 or more different reactions or sequences of reactions. In some cases, the plurality of different reactions or sequences of reactions may comprise 500 or more different reactions or sequences of reactions. In some cases, the plurality of different reactions or sequences of reactions may comprise 1000 or more different reactions or sequences of reactions. In some cases, a first reaction or sequence of reactions and a second reaction or sequence of reactions detect two different nucleic acid sequences. In some cases, each reaction or sequence of reactions from among a plurality of different reactions or sequences of reactions detects a different nucleic acid sequence. For example, a device may be configured to perform 40 different sequences of reactions designed to detect 40 different nucleic acid sequences from a single sample comprising 200 ng DNA (e.g., 200 ng DNA from a buccal swab). In such a case, each of the 40 different nucleic acid sequences could be used to determine the presence of a particular virus in the sample.

In some cases, a device is configured to automate a step. In some cases, a device automates a sample partitioning step. In some cases, a device automates a reaction step (e.g., by mixing a sample with reagents and heating to a temperature for a defined length of time). In some cases, the device automates every step following sample input. In some cases, a device may automate a plurality of reactions on a single input sample. In some cases, a device may automate, detect, and provide results for a plurality of reactions on a single input sample. In some cases, a device may automate, detect, and provide results for a plurality of reactions on a single sample in less than 2 hours. For example, a device may automate 100 separate amplification and DETECTR reactions on a sample comprising 400 ng DNA, detect and then provide the results of the reactions in less than 2 hours. In some cases, a device may automate, detect, and provide results for a plurality of reactions on a single sample in less than 1 hour. In some cases, a device may automate, detect, and provide results for a plurality of reactions on a single sample in less than 40 minutes. In some cases, a device may automate, detect, and provide results for a plurality of reactions on a single sample in less than 20 minutes. In some cases, a device may automate, detect, and provide results for a plurality of reactions on a single sample in less than 10 minutes. In some cases, a device may automate, detect, and provide results for a plurality of reactions on a single sample in less than 5 minutes. In some cases, a device may automate, detect, and provide results for a plurality of reactions on a single sample in less than 2 minutes.

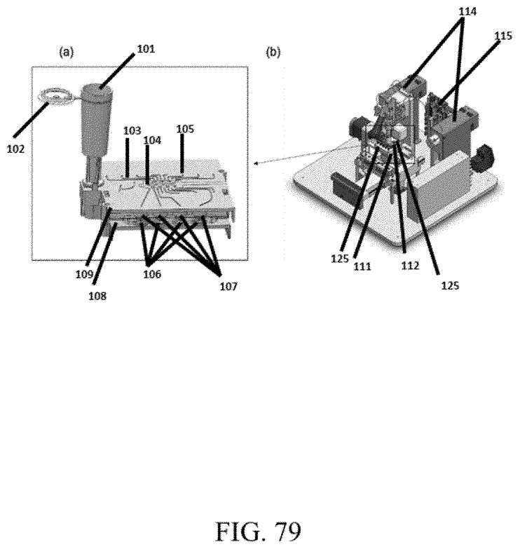

Microfluidic devices and detection manifolds for detection of viral infections. A microfluidic device of the present disclosure (e.g., a microfluidic device illustrated in FIG. 53A-B, FIG. 54A-B, FIG. 55A-D, FIG. 56A-D, FIG. 57A, FIG. 60, FIG. 76, FIG. 79, or FIG. 82-FIG. 92) may be used to detect the presence or absence of a coronavirus (e.g., a SARS-CoV-2 virus, a SARS-CoV virus, a MERS-CoV virus, a combination thereof, or a combination of any coronavirus strain and one or more other viruses or bacteria) in a biological sample. Detection of the coronavirus may be facilitated by a detection manifold (e.g., a detection manifold illustrated in FIG. 63A-B, FIG. 64B, FIG. 65, FIG. 81, FIG. 93, or FIG. 97). A biological sample may be collected from a subject, for example via a nasal swab or a buccal swab, and introduced into an amplification chamber of the microfluidic device. The chamber may comprise lysis buffer, amplification reagents, or both. In some embodiments, the biological sample may be contacted with a lysis buffer prior to introduction into the amplification chamber. In some embodiments, the amplification reagents may be introduced into the amplification chamber from an amplification reagent storage chamber. Introduction of the amplification reagents may be controlled by actuating a pump, a valve, or both via the detection manifold. The amplification reagents may comprise primers to amplify a target nucleic acid present in the coronavirus genome. If the target nucleic acid is present in the sample, the target nucleic acid may be amplified (e.g., by TMA, HDA, cHDA, SDA, LAMP, EXPAR, RCA, LCR, SMART, SPIA, MDA, NASBA, HIP, NEAR, or IMDA). The first chamber may be heated by the detection manifold. The amplified sample may be introduced into a detection chamber by actuating a pump, a valve, or both via the detection manifold. The amplified sample may pass through a sample metering channel. Detection reagents may be introduced into the detection channel from a detection reagent storage chamber by actuating a pump, a valve, or both via the detection manifold. The detection reagents may pass through a sample metering channel, a resistance channel, or both. The detection reagents may comprise a programmable nuclease, a guide nucleic acid directed to the target nucleic acid, and a labeled detector nucleic acid. A detection reaction may be performed in the detection channel by heating the detection channel via the detection manifold. The presence or absence of the target nucleic acid associated with the coronavirus may be detected in the detection channel using the detection manifold. The presence or absence of the coronavirus may be determined by measuring a detectable signal produced by cleavage of the detector nucleic acid by the programmable nuclease upon binding to the target nucleic acid.

A microfluidic device of the present disclosure (e.g., a microfluidic device illustrated in FIG. 53A-B, FIG. 54A-B, FIG. 55A-D, FIG. 56A-D, FIG. 57A, FIG. 60, FIG. 76, FIG. 79, or FIG. 82-FIG. 92) may be used to detect the presence or absence of an influenza virus (e.g., an influenza A virus or an influenza B virus) in a biological sample. Detection of the influenza virus may be facilitated by a detection manifold (e.g., a detection manifold illustrated in FIG. 63A-B, FIG. 64B, FIG. 65, FIG. 81, FIG. 93, or FIG. 97). A biological sample may be collected from a subject, for example via a nasal swab or a buccal swab, and introduced into an amplification chamber of the microfluidic device. The chamber may comprise lysis buffer, amplification reagents, or both. In some embodiments, the biological sample may be contacted with a lysis buffer prior to introduction into the amplification chamber. In some embodiments, the amplification reagents may be introduced into the amplification chamber from an amplification reagent storage chamber. Introduction of the amplification reagents may be controlled by actuating a pump, a valve, or both via the detection manifold. The amplification reagents may comprise primers to amplify a target nucleic acid present in the influenza viral genome. If the target nucleic acid is present in the sample, the target nucleic acid may be amplified (e.g., by TMA, HDA, cHDA, SDA, LAMP, EXPAR, RCA, LCR, SMART, SPIA, MDA, NASBA, HIP, NEAR, or IMDA). The first chamber may be heated by the detection manifold. The amplified sample may be introduced into a detection chamber by actuating a pump, a valve, or both via the detection manifold. The amplified sample may pass through a sample metering channel. Detection reagents may be introduced into the detection channel from a detection reagent storage chamber by actuating a pump, a valve, or both via the detection manifold. The detection reagents may pass through a sample metering channel, a resistance channel, or both. The detection reagents may comprise a programmable nuclease, a guide nucleic acid directed to the target nucleic acid, and a labeled detector nucleic acid. A detection reaction may be performed in the detection channel by heating the detection channel via the detection manifold. The presence or absence of the target nucleic acid associated with the influenza virus may be detected in the detection channel using the detection manifold. The presence or absence of the influenza virus may be determined by measuring a detectable signal produced by cleavage of the detector nucleic acid by the programmable nuclease upon binding to the target nucleic acid.

A microfluidic device of the present disclosure (e.g., a microfluidic device illustrated in FIG. 53A-B, FIG. 54A-B, FIG. 55A-D, FIG. 56A-D, FIG. 57A, FIG. 60, FIG. 76, FIG. 79, or FIG. 82-FIG. 92) may be used to detect the presence or absence of a respiratory syncytial virus in a biological sample. Detection of the respiratory syncytial virus may be facilitated by a detection manifold (e.g., a detection manifold illustrated in FIG. 63A-B, FIG. 64B, FIG. 65, FIG. 81, FIG. 93, or FIG. 97). A biological sample may be collected from a subject, for example via a nasal swab or a buccal swab, and introduced into an amplification chamber of the microfluidic device. The chamber may comprise lysis buffer, amplification reagents, or both. In some embodiments, the biological sample may be contacted with a lysis buffer prior to introduction into the amplification chamber. In some embodiments, the amplification reagents may be introduced into the amplification chamber from an amplification reagent storage chamber. Introduction of the amplification reagents may be controlled by actuating a pump, a valve, or both via the detection manifold. The amplification reagents may comprise primers to amplify a target nucleic acid present in the respiratory syncytial viral genome. If the target nucleic acid is present in the sample, the target nucleic acid may be amplified (e.g., by TMA, HDA, cHDA, SDA, LAMP, EXPAR, RCA, LCR, SMART, SPIA, MDA, NASBA, HIP, NEAR, or IMDA). The first chamber may be heated by the detection manifold. The amplified sample may be introduced into a detection chamber by actuating a pump, a valve, or both via the detection manifold. The amplified sample may pass through a sample metering channel. Detection reagents may be introduced into the detection channel from a detection reagent storage chamber by actuating a pump, a valve, or both via the detection manifold. The detection reagents may pass through a sample metering channel, a resistance channel, or both. The detection reagents may comprise a programmable nuclease, a guide nucleic acid directed to the target nucleic acid, and a labeled detector nucleic acid. A detection reaction may be performed in the detection channel by heating the detection channel via the detection manifold. The presence or absence of the target nucleic acid associated with the respiratory syncytial virus may be detected in the detection channel using the detection manifold. The presence or absence of the respiratory syncytial virus may be determined by measuring a detectable signal produced by cleavage of the detector nucleic acid by the programmable nuclease upon binding to the target nucleic acid.

### Kit

Disclosed herein are kits, reagents, methods, and systems for use to detect a target nucleic acid. The target nucleic acid can be from a coronavirus, such as SARS-CoV-2, 229E (alpha coronavirus), NL63 (alpha coronavirus), OC43 (beta coronavirus), HKU1 (beta coronavirus), MERS-CoV, or SARS-CoV. In some embodiments, the target nucleic acid is from the SARS-CoV-2 coronavirus. Any nucleic acid of the SARS-CoV-2 can be assayed for using the compositions and methods disclosed herein and used in a kit as described herein. In some embodiments, the target nucleic acid comprises the N gene or the E gene of coronavirus and can be assayed for using the compositions and methods disclosed herein and used in a kit as described herein. In some embodiments, the kit comprises the reagents and a support medium. The reagent may be provided in a reagent chamber or on the support medium. Alternatively, the reagent may be placed into the reagent chamber or the support medium by the individual using the kit. Optionally, the kit further comprises a buffer and a dropper. The reagent chamber be a test well or container. The opening of the reagent chamber may be large enough to accommodate the support medium. The buffer may be provided in a dropper bottle for ease of dispensing. The dropper can be disposable and transfer a fixed volume. The dropper can be used to place a sample into the reagent chamber or on the support medium.

In some embodiments, a kit for detecting a target nucleic acid comprising a support medium; a guide nucleic acid targeting a target nucleic acid segment; a programmable nuclease capable of being activated when complexed with the guide nucleic acid and the target nucleic acid segment; and a single stranded detector nucleic acid comprising a detection moiety, wherein the detector nucleic acid is capable of being cleaved by the activated nuclease, thereby generating a first detectable signal.

In some embodiments, a kit for detecting a target nucleic acid comprising a PCR plate; a guide nucleic acid targeting a target nucleic acid segment; a programmable nuclease capable of being activated when complexed with the guide nucleic acid and the target nucleic acid segment; and a single stranded detector nucleic acid comprising a detection moiety, wherein the detector nucleic acid is capable of being cleaved by the activated nuclease, thereby generating a first detectable signal. The wells of the PCR plate can be pre-aliquoted with the guide nucleic acid targeting a target nucleic acid segment, a programmable nuclease capable of being activated when complexed with the guide nucleic acid and the target sequence, and at least one population of a single stranded detector nucleic acid comprising a detection moiety. A user can thus add the biological sample of interest to a well of the pre-aliquoted PCR plate and measure for the detectable signal with a fluorescent light reader or a visible light reader.

In some instances, such kits may include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers.

The kit or systems described herein contain packaging materials. Examples of packaging materials include, but are not limited to, pouches, blister packs, bottles, tubes, bags, containers, bottles, and any packaging material suitable for intended mode of use.

A kit typically includes labels listing contents and/or instructions for use, and package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### EXAMPLES

The following examples are illustrative and non-limiting to the scope of the devices, methods, reagents, systems, and kits described herein.

### Example 1

### RT-LAMP DETECTR Reactions for Detection of Coronavirus

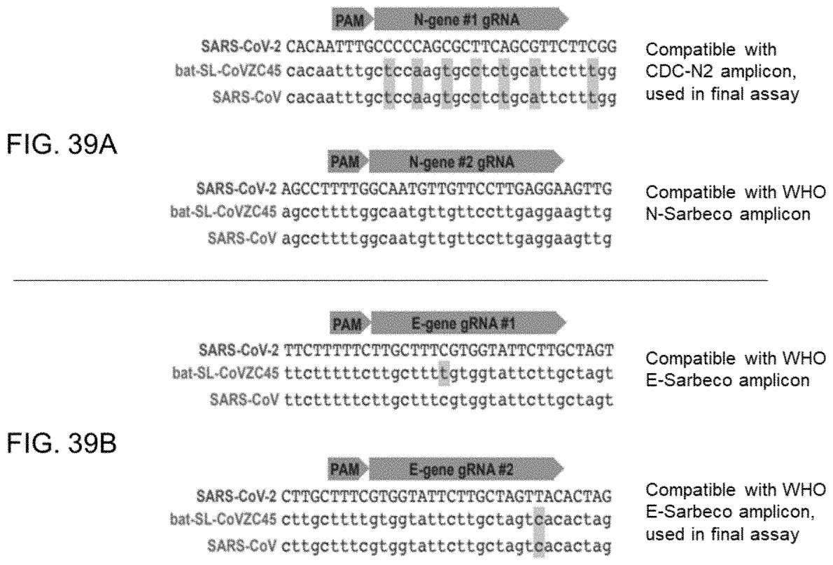

This example describes RT-LAMP DETECTR reactions for the detection of coronavirus. SARS-CoV-2 target sequences were designed using all available genomes available from GISAID. Briefly, viral genomes were aligned using Clustal Omega. Next, LbCas12a target sites on the SARS-CoV-2 genome were filtered against SARS-CoV, two bat-SARS-like-CoV genomes and common human coronavirus genomes. Compatible target sites were finally compared to those used in current protocols from the CDC and WHO. LAMP primers for SARS-CoV-2 were designed against regions of the N-gene and E-gene using PrimerExplorer v5 (https://primerexplorer.jp/e/). FIG. 39A shows a sequence alignment of the target sites targeted by the N-gene gRNA for three coronavirus strains. The N gene gRNA #1 is compatible with the CDC-N2 amplicon, the N gene gRNA #2 is compatible with WHO N-Sarbeco amplicon. FIG. 39B shows a sequence alignment of the target sites targeted by the E-gene gRNA for three coronavirus strains. The two E gene gRNAs tested (E gene gRNA #1 and E gene gRNA #2) are compatible with the WHO E-Sarbeco amplicon. RNase P POP7 primers were originally published by Curtis, et al. (2018) and a compatible gRNA was designed to function with these primer sets.

Target RNAs were generated from synthetic gene fragments of the viral genes of interest. First a PCR step was performed on the synthetic gene fragment with a forward primer that contained a T7 promoter. Next, the PCR product was used as the template for an in-vitro transcription (IVT) reaction at 37° C. for 2 hours. The IVT reaction was then treated with TURBO DNase (Thermo) for 30 minutes at 37° C., followed by a heat-denaturation step at 75° C. for 15 minutes. RNA was purified using RNA Clean and Concentrator 5 columns (Zymo Research). RNA was quantified by Nanodrop and Qubit and diluted in nuclease-free water to working concentrations.

DETECTR assays were performed using RT-LAMP for pre-amplification of viral or control RNA targets and LbCas12a for the trans-cleavage assay. RT-LAMP was prepared with a MgSO₄ concentration of 6.5 mM and a final volume of 10 μL. LAMP primers were added at a final concentration of 0.2 μM for F3 and B3, 1.6 μM for FIP and BIP, and 0.8 μM for LF and LB. Reactions were performed independently for N-gene, E-gene, and RNase P using 2 μL of input RNA at 62° C. for 20 minutes.

For LbCas12a (SEQ ID NO: 18) trans-cleavage, 50 nM LbCas12a (available from NEB) was pre-incubated with 62.5 nM gRNA in 1×NEBuffer 2.1 for 30 minutes at 37° C. After formation of the RNA-protein complex, the lateral flow cleavage reporter (/56-FAM/TTATTATT/3Bio/, IDT) was added to the reaction at a final concentration of 500 nM. RNA-protein complexes were used immediately or stored at 4° C. for up to 24 hours before use.

After completion of the pre-amplification step, 2 μL of amplicon was combined with 18 μL of LbCas12a-gRNA complex and 80 μL of 1×NEBuffer 2.1. The 100 μL LbCas12a trans-cleavage assay was allowed to proceed for 10 minutes at 37° C.

A lateral flow strip (Milenia HybriDetect 1, TwistDx) was then added to the reaction tube and a result was visualized after approximately 2-3 minutes. A single band, close to the sample application pad indicated a negative result, whereas a single band close to the top of the strip or two bands indicated a positive result.

The patient optimized DETECTR assays were performed using RT-LAMP method as described above with the following modifications: A DNA binding dye, SYTO9 (Thermo Fisher Scientific), was included in the reaction to monitor the amplification reaction and the incubation time was extended to 30 minutes to capture data from lower titre samples.

The fluorescence based patient optimized LbCas12a trans-cleavage assays were performed as described above with modifications; 40 nM LbCas12a was pre-incubated with 40 nM gRNA, after which 100 nM of a fluorescent reporter molecule compatible with detection in the presence of the SYTO9 dye (/5Alex594N/TTATTATT/3IAbRQSp/) was added to the complex. 2 μL of amplicon was combined with 18 μL of LbCas12a-gRNA complex in a black 384-well assay plate and monitored for fluorescence using a Tecan plate reader.

### Example 2

### Screening of Primer Sets for Amplification of a SARS-CoV-2 Target Site

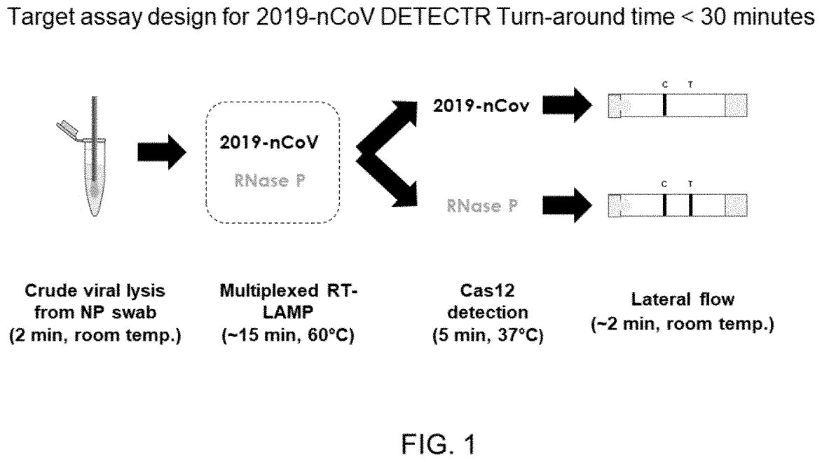

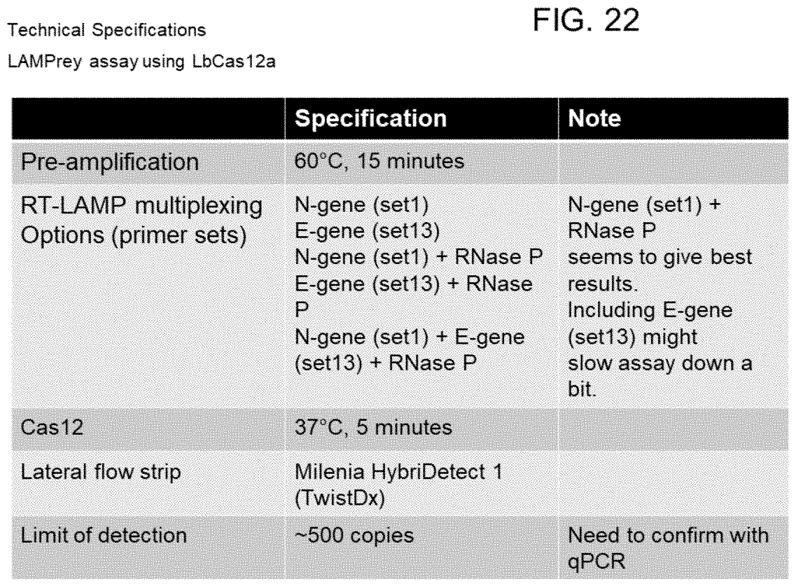

This example describes the screening of primer sets for amplification of a SARS-CoV-2 target site. A region of the coronavirus RNA genome corresponding to the viral N-gene was amplified using different LAMP primer sets (set1 through set 11). Samples containing either 1.5 μM, 5 fM, or 0 fM SARS-CoV-2 RNA were amplified with each primer set. SARS-CoV-2 RNA in each sample was reverse transcribed using a warmstart reverse transcriptase (“Warmstart RTx”) and LAMP amplified using a Bst 2.0 DNA polymerase. The assay was performed at 60 degrees C. for 60 minutes. FIG. 1 illustrates schematically the steps of preparing and detecting a sample using a RT-LAMP and Cas12 DETECTR reactions. FIG. 22 shows technical specifications and assay conditions for detection of coronavirus using reverse transcription and loop-mediated isothermal amplification (RT-LAMP) and Cas12 detection.

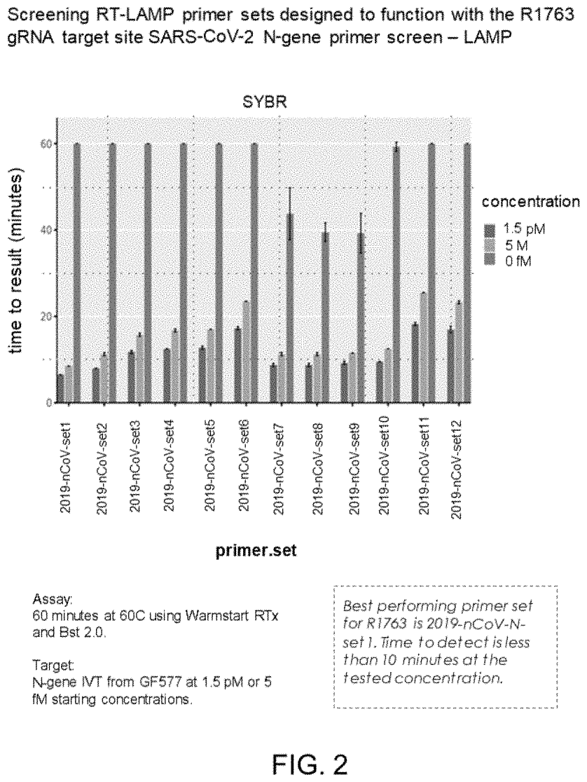

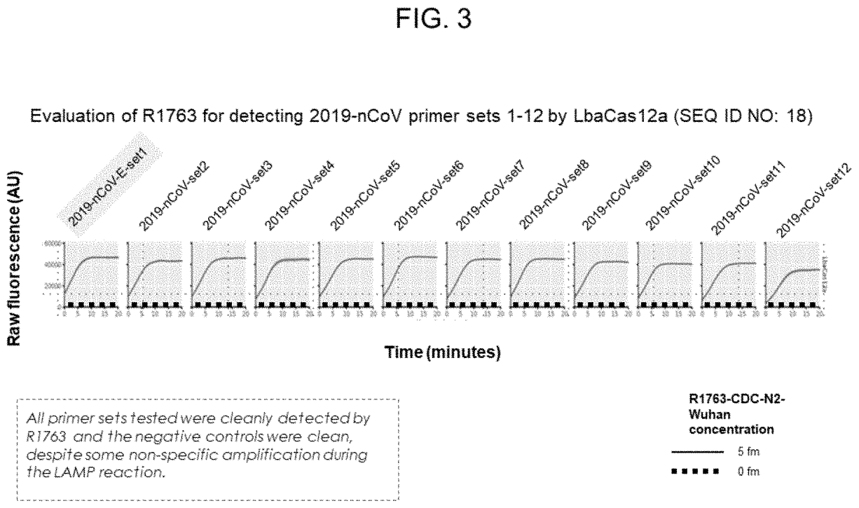

A DETECTR assay was performed on each amplified sample, and the time to result was determined. Sequences were detected using a gRNA sequence corresponding to R1763 directed to the N-gene of SARS-CoV-2 and a Cas12 programmable nuclease corresponding to LbCas12a. The DETECTR assay was sensitive for the amplified SARS-CoV-2 target sequence for all tested primer sets. Sequences of the gRNAs used in this example are provided in TABLE 6. FIG. 2 shows the DETECTR assay results of the SARS-CoV-2 N-gene amplified with different primer sets (“2019-nCoV-set1” through “2019-nCoV-set12”) and detected using LbCas12a and a gRNA directed to the N-gene of SARS-CoV-2 (“R1763,” SEQ ID NO: 171). A lower time to result is indicative of a positive result. For all primer sets, the time to result was lower for samples with more of the target sequence, indicating that the assay was sensitive for the target sequence. FIG. 3 shows the individual traces of the DETECTR reactions plotted in FIG. 2 for the 0 fM and 5 fM samples. In each plot, the 0 fM trace is not visible above the baseline, indicating that there little to no non-specific detection. The best performing primer set for R1763 (SEQ ID NO: 171) was SARS-CoV-2-N-set1. Time to detect was less than 10 minutes at the tested concentration.

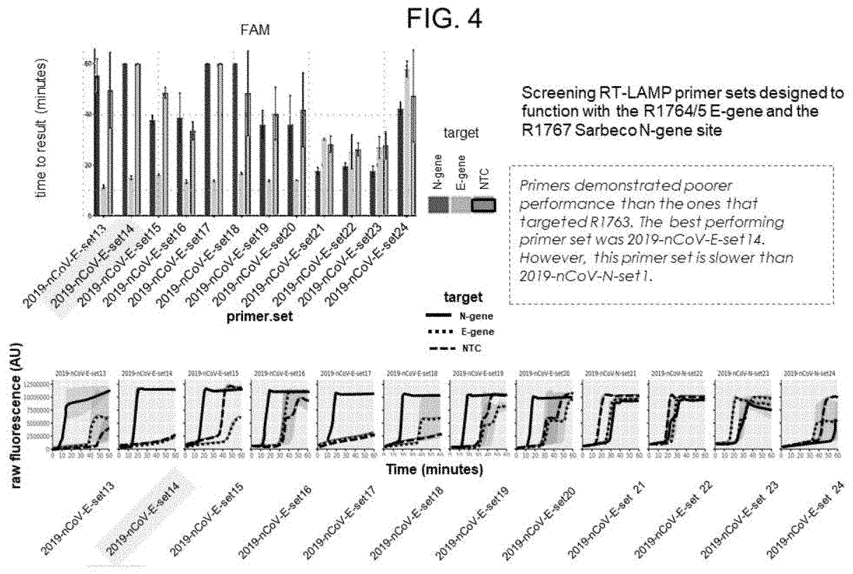

In a second assay, primer sets directed to the E-gene of Sarbeco (detected with gRNAs R1764 and R1765) and the N-gene of Sarbeco (detected with R1767). FIG. 4 shows the time to result of a DETECTR reaction on samples containing either the N-gene, the E-gene, or no target (“NTC”). Samples were amplified using primer sets directed to the E-gene of SARS-CoV-2 (“2019-nCoV-E-set13” through “2019-nCoV-E-set20”) or to the N-gene of SARS-CoV-2 (“2019-nCoV-N-set21” through “2019-nCoV-N-set24”). Target site sequences are provided in TABLE 7. The best performing primer set was SARS-CoV-2-E-set14. The presence of the SARS-CoV-2 N-gene was detected using the R1767 N-gene gRNA (SEQ ID NO: 175) and the presence of the SARS-CoV-2 E-gene was detected using either the R1764 E-gene gRNA (SEQ ID NO: 172) or the R1765 E-gene gRNA (SEQ ID NO: 173).

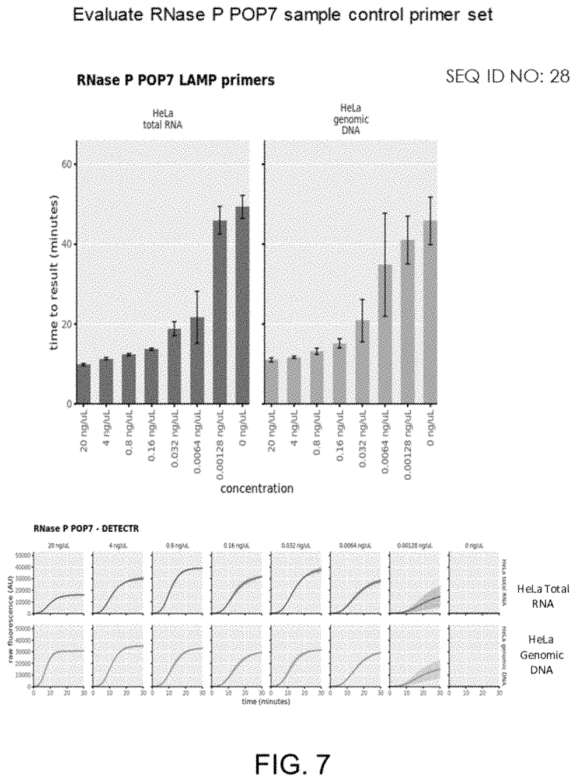

A control primer set for amplifying RNase P was also tested. FIG. 7 shows the amplification of RNase P using a POP7 sample primer set. Samples were amplified using LAMP. DETECTR reactions were performed using a gRNA directed to RNase P (“R779,” SEQ ID NO: 178) and a Cas12 variant (SEQ ID NO: 28). Samples contained either HeLa total RNA or HeLa genomic DNA.

### Example 3

### Specificity of Detection of a SARS-CoV-2 Target Nucleic Acid

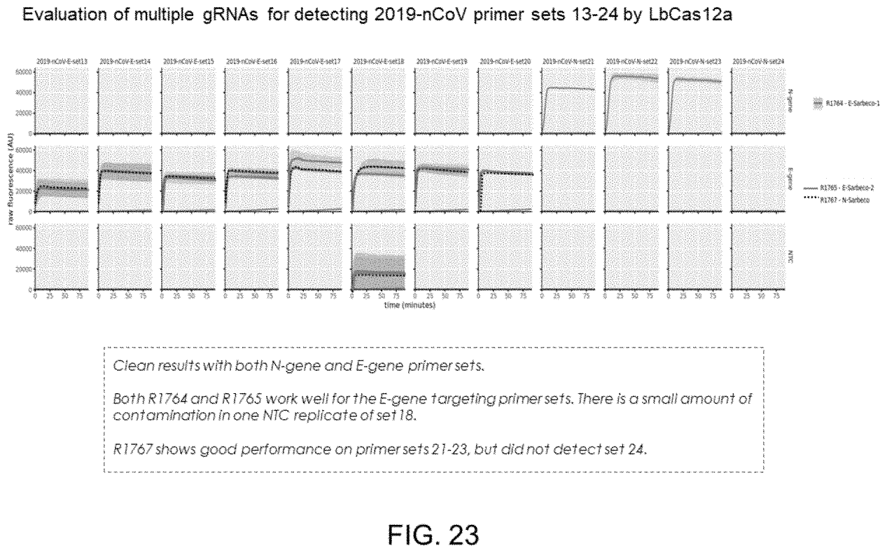

This example describes the specificity of detection of a SARS-CoV-2 target nucleic acid. A sample containing target RNA corresponding to SARS-CoV-2 was amplified as using primer set 1 as described in EXAMPLE 2. gRNAs were screened for compatibility with different primer sets designed to amplify either the N-gene or the E-gene of SARS-CoV-2. FIG. 23 shows the results of a DETECTR assay evaluating multiple gRNAs for detecting SARS-CoV-2 using LbCas12a. Target nucleic acid sequences were amplified using primer sets to amplify the SARS-CoV-2 E-gene (“2019-nCoV-E-set13” through “2019-nCoV-E-set20” or the SARS-CoV-2 N-gene (“2019-nCoV-N-set2l” through “2019-nCoV-N-set24”). The gRNA corresponding to SEQ ID NO: 172 (“R1764-E-Sarbeco-1) and the gRNA corresponding to SEQ ID NO: 173 (“R1765-E-Sarbeco-2”) were able to detect target sequences amplified using LAMP primer sets directed to the E-gene of SARS-CoV-2. The gRNA corresponding to SEQ ID NO: 175 (“R1767-N-Sarbeco”) was ample to detect target sequences amplified using most LAMP primer sets directed to the N-gene of SARS-CoV-2.

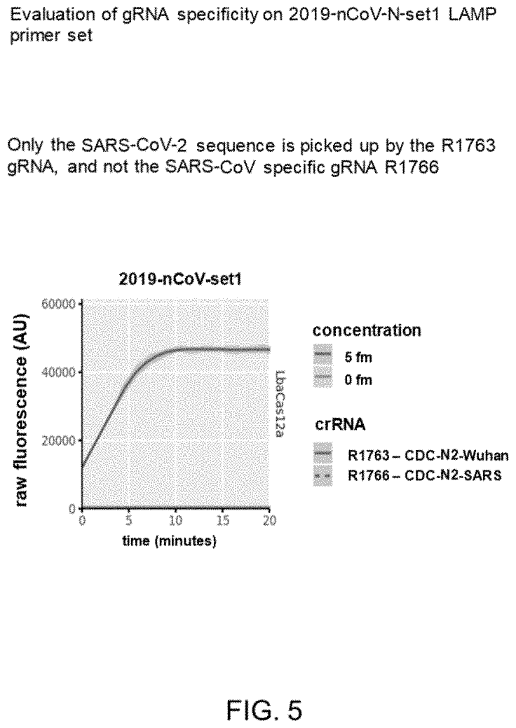

Samples containing either 5 fM or 0 fM SARS-CoV-2 RNA were detected using a DETECTR assay. Samples were detected using LbCas12a and either a gRNA R1763 directed to the N-gene of SARS-CoV-2 or a gRNA R1766 directed to the N-gene of SARS-CoV. Sequences of the gRNAs used in this example are provided in TABLE 6. FIG. 5 shows the DETECTR assay results of the SARS-CoV-2 N-gene amplified with primer set 1 (“2019-nCoV-set1”) and detected using LbCas12a (SEQ ID NO: 18) and either a gRNA directed to the N-gene of SARS-CoV-2 (“R1763-CDC-N2-Wuhan,” SEQ ID NO: 171) or a gRNA directed to the N-gene of SARS-CoV (“R1766-CDC-N2-SARS,” SEQ ID NO: 174).

FIG. 11 schematically illustrates the sequence of the CDC-N2 target site used for detecting the N-2 gene of SARS-CoV-2 in this assay. Target site sequences are provided in TABLE 7.

TABLE 6
Exemplary gRNA Sequences for Detection of Coronaviruses

| SEQ ID NO: | gRNA | Target | Sequence |
|---|---|---|---|
| SEQ ID NO: 171 | R1763 | CDC-N2-Wuhan | UAAUUUCUACUAAGUGUAGAUCCCC |
| | | | CAGCGCUUCAGCGUUC |
| SEQ ID NO: 172 | R1764 | E-Sarbeco-1 | UAAUUUCUACUAAGUGUAGAUUUGC |
| | | | UUUCGUGGUAUUCUUG |
| SEQ ID NO: 173 | R1765 | E-Sarbeco-2 | UAAUUUCUACUAAGUGUAGAUGUGG |
| | | | UAUUCUUGCUAGUUAC |
| SEQ ID NO: 174 | R1766 | CDC-N2-SARS | UAAUUUCUACUAAGUGUAGAUCUCC |
| | | | AAGUGCCUCUGCAUUC |
| SEQ ID NO: 175 | R1767 | N-Sarbeco-1 | UAAUUUCUACUAAGUGUAGAUGCAA |
| | | | UGUUGUUCCUUGAGGA |
| SEQ ID NO: 176 | R1768 | ORF1ab-Wuhan | UAAUUUCUACUAAGUGUAGAUCACA |
| | | | UACCGCAGACGGUACA |
| SEQ ID NO: 177 | R1769 | CDC-RNaseP | UAAUUUCUACUAAGUGUAGAUGACC |
| | | | UGCGAGCGGGUUCUGA |
| SEQ ID NO: 178 | R779 | RNaseP POP7 | UAAUUUCUACUAAGUGUAGAUAAUU |
| | | | ACUUGGGUGUGACCCU |
| SEQ ID NO: 218 | R1965 | RNaseP POP7 v2 | UAAUUUCUACUAAGUGUAGAUUUAC |
| | | | AUGGCUCUGGUCCGAG |
| SEQ ID NO: 219 | R780 | RNaseP POP7 v3 | UAAUUUCUACUAAGUGUAGAUGGCT |
| | | | TCCAGGGAACAGGCCT |

### Example 4

### Limit of Detection of SARS-CoV-2

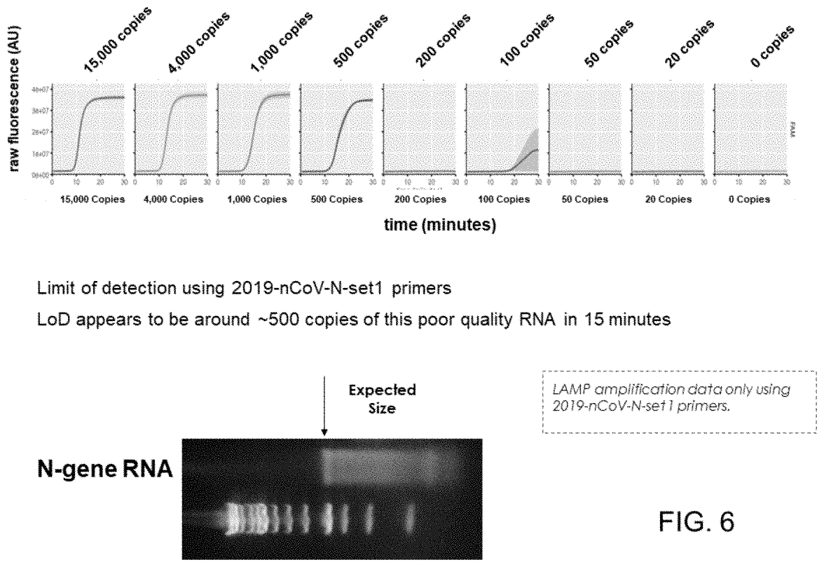

This example describes the limit of detection of SARS-CoV-2. Samples containing decreasing copy numbers of SARS-CoV-2 target nucleic acid were detected using a DETECTR reaction. FIG. 6 shows the results of a DETECTR reaction to determine the limit of detection of SARS-CoV-2 in a DETECTR reaction amplified using a primer set directed to the N-gene of SARS-CoV-2 (“2019-nCoV-N-set1”). Samples containing either 15,000, 4,000, 1,000, 500, 200, 100, 50, 20, or 0 copies of a SARS-CoV-2 N-gene target nucleic acid were detected. A gel of the N-gene RNA is shown below. Samples were detected using a gRNA directed to the N-gene of SARS-CoV-2 (SEQ ID NO: 171).

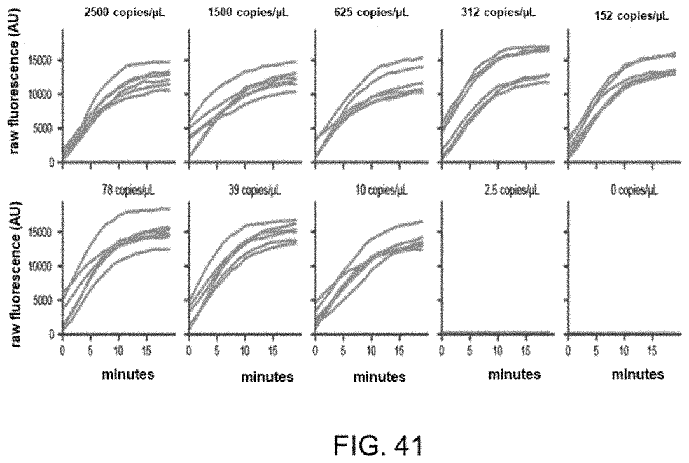

FIG. 41 shows DETECTR analysis of SARS-CoV-2 identifies down to 10 viral genomes in approximately 30 min (20 min amplification, 10 min DETECTR). Duplicate LAMP reactions were amplified for twenty min followed by LbCas12a DETECTR analysis.

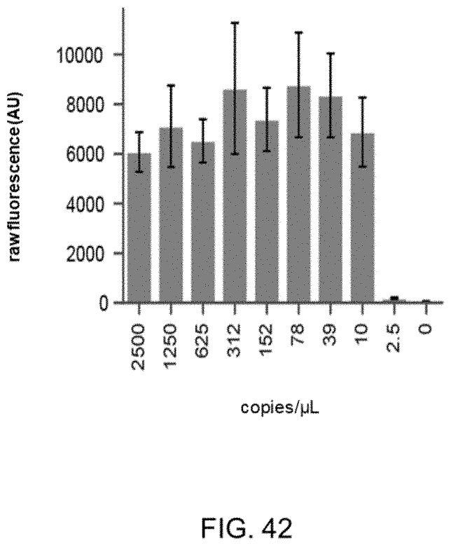

FIG. 42 shows the raw fluorescence at 5 minutes for the LbCas12a DETECTR analysis provided in FIG. 41. The limit of detection of the SARS-CoV-2 N-gene was determined to be 10 viral genomes per reaction (n=6).

### Example 5

### Multiplexing SARS-CoV-2 Primer Sets for Detection of SARS-CoV-2

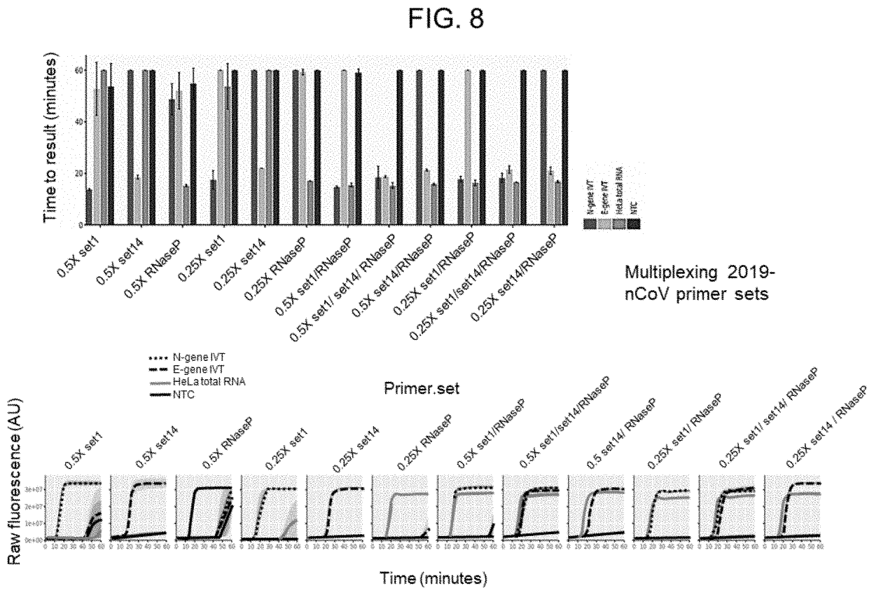

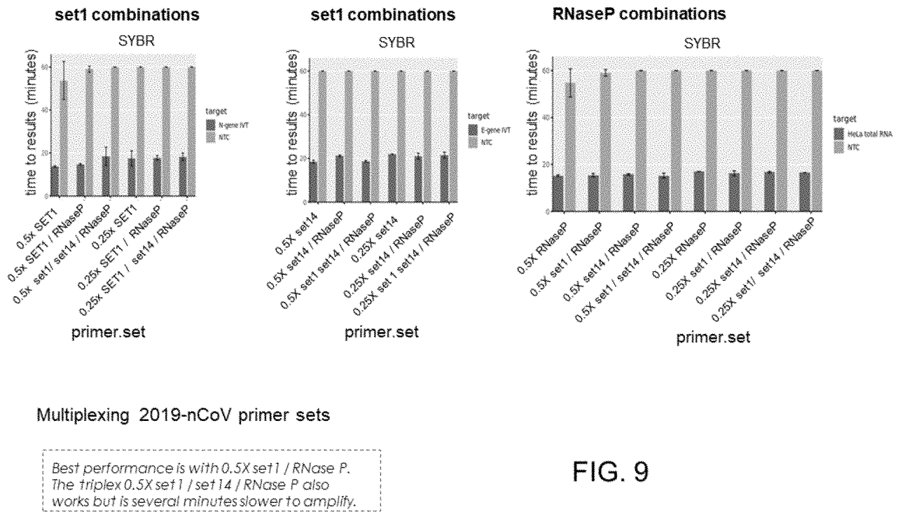

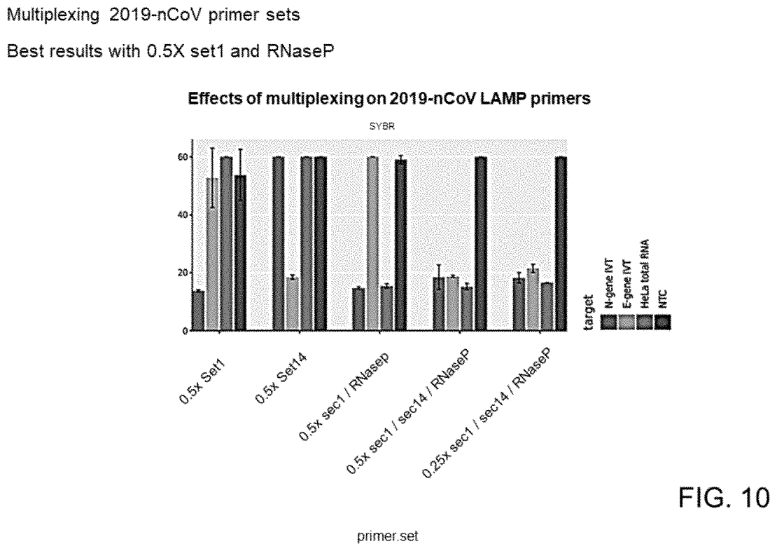

This example describes multiplexing SARS-CoV-2 primer sets for detection of SARS-CoV-2. Samples containing target nucleic acids were amplified using a combination of primer sets directed to one or more of SARS-CoV-2 or RNase P. Primer sets directed to SARS-CoV-2 are denoted by “set” with a number. FIG. 8 shows the time to result of a multiplexed DETECTR reaction. Samples contained either in vitro transcribed N-gene of SARS-CoV-2 (“N-gene IVT”), in vitro transcribed E-gene of SARS-CoV-2 (“E-gene IVT”), HeLa total RNA, or no target (“NTC”). Samples were amplified using one or more primer sets directed to the SARS-CoV-2 N-gene (“set1”), the SARS-CoV-2 E-gene (“set14”), or RNase” (“RNaseP”). FIG. 9 shows the time to results of a multiplexed DETECTR reaction with different combinations of primer sets directed to either SARS-CoV-2 N-gene (“set1”), SARS-CoV-2 E-gene (“set14”), or RNase P. Samples containing in vitro transcribed N-gene of SARS-CoV-2 (left, “N-gene IVT”) or in vitro transcribed E-gene of SARS-CoV-2 (right, “E-gene IVT”) were tested. FIG. 10 shows the time to result of a multiplexed DETECTR reaction with the best performing primer set combinations from FIG. 8 and FIG. 9.

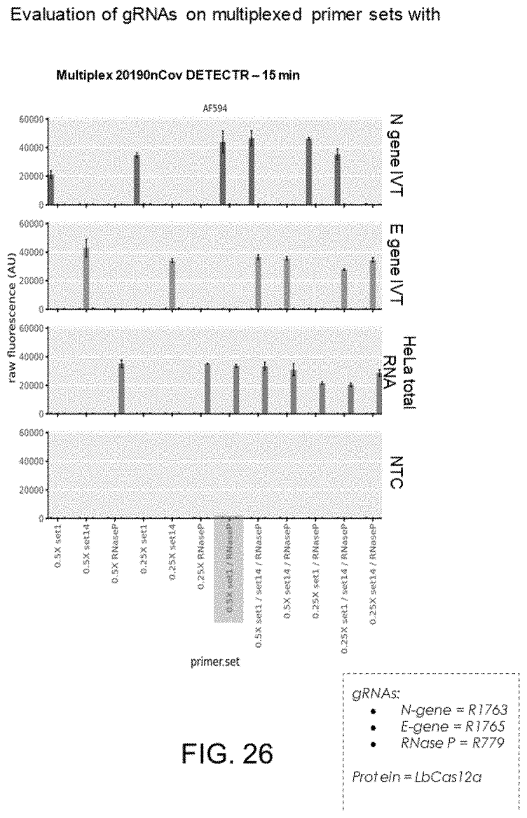

FIG. 26 shows the results of a DETECTR assay evaluating LAMP primer sets for their utility in multiplexed amplification of SARS-CoV-2 targets. Samples were amplified with one or more primer sets directed to the SARS-CoV-2 N-gene (“set1”) or the SARS-CoV-2 E-gene (“set14”), or RNase P (“RNaseP”). Samples were detected with either a gRNA directed to the N-gene of SARS-CoV-2 (SEQ ID NO: 171, “N-gene”), the E-gene of SARS-CoV-2 (SEQ ID NO: 173, “E-gene”), or RNase P (SEQ ID NO: 178).

### Example 6

### Sensitivity of a DETECTR Assay to Distinguish Three Coronaviruses

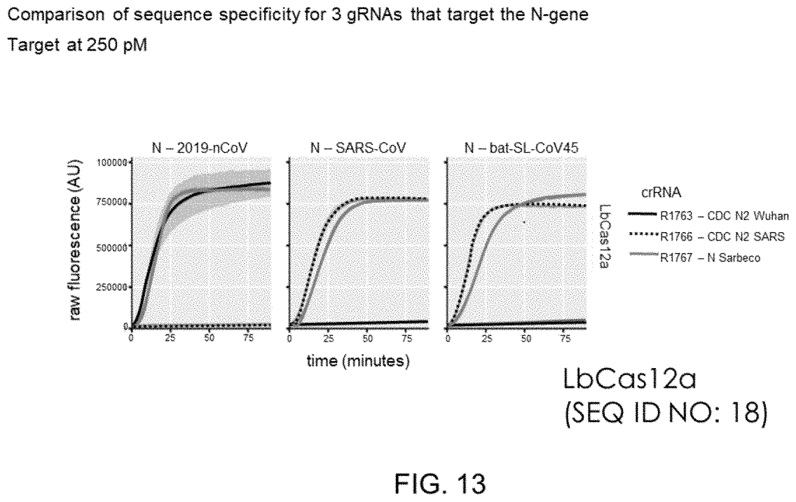

This example describes the sensitivity of a DETECTR assay to distinguish three coronaviruses. Samples containing 250 μM of either RNA corresponding to the N-gene of SARS-CoV-2, the N-gene of SARS-CoV, or the N-gene of bat-SL-CoV45. Samples were amplified at detected as described in EXAMPLE 2. Samples were detected using each of a gRNA directed to the N-gene of SARS-CoV-2 (“R1763”), a gRNA directed to the N-gene of SARS-CoV (“R1766”), or a gRNA directed to the N-gene of a Sarbeco coronavirus (“R1767”). Sequences of the gRNAs used in this example are provided in TABLE 6. FIG. 12 schematically illustrates the sequence of a region of the SARS-CoV-2 N-gene (“N-Sarbeco”) target site. Target site sequences are provided in TABLE 7. FIG. 13 shows the results of a DETECTR assay to determine the sensitivity of gRNAs directed to either N-gene of SARS-CoV-2 (“R1763,” SEQ ID NO: 171), the N-gene of SARS-CoV (“R1766,” SEQ ID NO: 174), or the N-gene of a Sarbeco coronavirus (“R1767,” SEQ ID NO: 175) for samples containing either the N-gene of SARS-CoV-2 (“N-2019-nCoV”), the N-gene of SARS-CoV (“N-SARS-CoV”), or the N-gene of bat-SL-CoV45 (“N-bat-SL-CoV45”). SARS-CoV-2, SARS-CoV, and bat-SL-CoV45 are strains of sarbeco coronavirus. Samples were detected using LbCas12a (SEQ ID NO: 18).

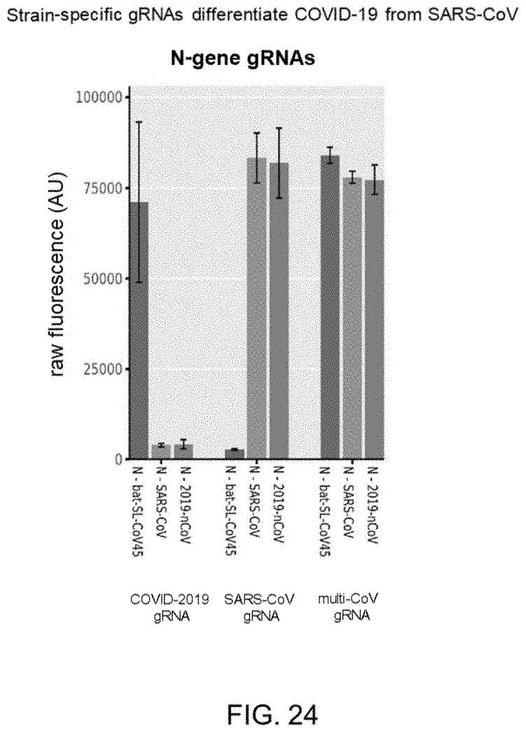

FIG. 24 shows the results of a DETECTR assay evaluating multiple gRNAs for their utility in distinguishing between three different strains of coronavirus, SARS-CoV-2 (“COVID-2019”), SARS-CoV, or bat-SL-CoV45. Samples containing N-gene amplicons of either SARS-CoV-2 (“N-2019-nCoV”), SARS-CoV (“N-SARS-CoV”), or bat-SL-CoV45 (“N-bat-SL-CoV45”) were tested. Samples were detected with a gRNA directed to the N-gene of SARS-CoV-2 (SEQ ID NO: 171, “COVID-2019 gRNA”), a gRNA directed to the N-gene of SARS-CoV (SEQ ID NO: 174, “SARS-CoV gRNA”), or a gRNA directed to the N-gene of multiple coronavirus species (SEQ ID NO: 175, “multi-CoV gRNA”).

TABLE 7
Exemplary Coronavirus N-Gene and E-Gene Gene Fragments

| SEQ ID NO: | Target | Sequence |
|---|---|---|
| SEQ ID NO: | 2019- | CCAAATTGGCTACTACCGAAGAGCTACCAGACGAATTCGTGGTGG |
| 179 | nCoV | TGACGGTAAAATGAAAGATCTCAGTCCAAGATGGTATTTCTACTA |
| | N-gene | CCTAGGAACTGGGCCAGAAGCTGGACTTCCCTATGGTGCTAACAA |
| | | AGACGGCATCATATGGGTTGCAACTGAGGGAGCCTTGAATACAC |
| | | CAAAAGATCACATTGGCACCCGCAATCCTGCTAACAATGCTGCAA |
| | | TCGTGCTACAACTTCCTCAAGGAACAACATTGCCAAAAGGCTTCT |
| | | ACGCAGAAGGGAGCAGAGGCGGCAGTCAAGCCTCTTCTCGTTCCT |
| | | CATCACGTAGTCGCAACAGTTCAAGAAATTCAACTCCAGGCAGCA |
| | | GTAGGGGAACTTCTCCTGCTAGAATGGCTGGCAATGGCGGTGATG |
| | | CTGCTCTTGCTTTGCTGCTGCTTGACAGATTGAACCAGCTTGAGA |
| | | GCAAAATGTCTGGTAAAGGCCAACAACAACAAGGCCAAACTGTC |
| | | ACTAAGAAATCTGCTGCTGAGGCTTCTAAGAAGCCTCGGCAAAA |
| | | ACGTACTGCCACTAAAGCATACAATGTAACACAAGCTTTCGGCAG |
| | | ACGTGGTCCAGAACAAACCCAAGGAAATTTTGGGGACCAGGAAC |
| | | TAATCAGACAAGGAACTGATTACAAACATTGGCCGCAAATTGCA |
| | | CAATTTGCCCCCAGCGCTTCAGCGTTCTTCGGAATGTCGCGCATT |
| | | GGCATGGAAGTCACACCTTCGGGAACGTGGTTGACCTACACAGGT |
| | | GCCATCAAATTGGATGACAAAGATCCAAATTTCAAAGATCAAGTC |
| | | ATTTTGCTGAATAAGCATATTGACGCATACAAAACATTCCCACCA |
| | | ACAGAGCCTAAAAAGGACAAAAAGAAGAAGGCTGATGAAACTC |
| | | AAGCCTTACCGCAGAGACAGAAGAAACAGCAAACTGTG |
| SEQ ID NO: | SARS- | CCAAATTGGCTACTACCGAAGAGCTACCCGACGAGTTCGTGGTGG |
| 180 | CoV N- | TGACGGCAAAATGAAAGAGCTCAGCCCCAGATGGTACTTCTATTA |
| | gene | CCTAGGAACTGGCCCAGAAGCTTCACTTCCCTACGGCGCTAACAA |
| | | AGAAGGCATCGTATGGGTTGCAACTGAGGGAGCCTTGAATACAC |
| | | CCAAAGACCACATTGGCACCCGCAATCCTAATAACAATGCTGCCA |
| | | CCGTGCTACAACTTCCTCAAGGAACAACATTGCCAAAAGGCTTCT |
| | | ACGCAGAGGGAAGCAGAGGCGGCAGTCAAGCCTCTTCTCGCTCC |
| | | TCATCACGTAGTCGCGGTAATTCAAGAAATTCAACTCCTGGCAGC |
| | | AGTAGGGGAAATTCTCCTGCTCGAATGGCTAGCGGAGGTGGTGA |
| | | AACTGCCCTCGCGCTATTGCTGCTAGACAGATTGAACCAGCTTGA |
| | | GAGCAAAGTTTCTGGTAAAGGCCAACAACAACAAGGCCAAACTG |
| | | TCACTAAGAAATCTGCTGCTGAGGCATCTAAAAAGCCTCGCCAAA |
| | | AACGTACTGCCACAAAACAGTACAACGTCACTCAAGCATTTGGG |
| | | AGACGTGGTCCAGAACAAACCCAAGGAAATTTCGGGGACCAAGA |
| | | CCTAATCAGACAAGGAACTGATTACAAACATTGGCCGCAAATTGC |
| | | ACAATTTGCTCCAAGTGCCTCTGCATTCTTTGGAATGTCACGCATT |
| | | GGCATGGAAGTCACACCTTCGGGAACATGGCTGACTTATCATGGA |
| | | GCCATTAAATTGGATGACAAAGATCCACAATTCAAAGACAACGT |
| | | CATACTGCTGAACAAGCACATTGACGCATACAAAACATTCCCACC |
| | | AACAGAGCCTAAAAAGGACAAAAAGAAAAAGACTGATGAAGCT |
| | | CAGCCTTTGCCGCAGAGACAAAAGAAGCAGCCCACTGTG |
| SEQ ID NO: | bat-SL- | CCAAATTGGCTACTACCGTAGAGCTACCAGACGAATTCGTGGTGG |
| 181 | CoVZC | TGACGGTAAAATGAAAGAGCTCAGCCCCAGATGGTATTTTTACTA |
| | 45 N- | TCTAGGAACTGGACCAGAAGCTGGACTTCCCTATGGTGCTAACAA |
| | gene | AGAAGGCATCATATGGGTTGCAACTGAGGGAGCCTTAAACACAC |
| | | CGAAAGACCACATTGGCACCCGCAATCCTGCTAACAATGCTGCAA |
| | | TCGTGCTACAACTTCCTCAAGGAACAACATTGCCAAAAGGCTTCT |
| | | ACGCAGAAGGGAGCAGAGGCGGCAGTCAAGCTTCTTCACGCTCC |
| | | TCATCACGTAGTCGCAACAGTTCAAGAAACTCAACTCCAGGCAGC |
| | | AGTAGGGGAACTTCTCCTGCTAGAATGGCTGGCAATGGCGGTGAC |
| | | ACTGCTCTTGCTTTGCTGCTGCTAGATAGGTTGAACCAGCTTGAG |
| | | AACAAAGTATCTGGCAAAGGCCAACAACAACAGGGCCAAACTGT |
| | | CACTAAGAAATCTGCTGCTGAGGCATCTAAAAAGCCTCGCCAAA |
| | | AACGTACTGCTACAAAACAGTACAACGTCACTCAAGCATTTGGGA |
| | | GACGTGGTCCAGAACAAACCCAAGGAAATTTTGGGGACCAAGAA |
| | | TTAATCAGACAAGGAACTGATTACAAACATTGGCCGCAAATTGCA |
| | | CAATTTGCTCCAAGTGCCTCTGCATTCTTTGGAATGTCACGCATTG |
| | | GCATGGAAGTCACACCTTCGGGAACATGGCTGACTTATCATGGAG |
| | | CCATTAAATTGGATGACAAAGATCCACAATTCAAAGATAACGTCA |
| | | TACTGCTGAATAAGCACATTGACGCATACAAAACATTCCCACCAA |
| | | CAGAGCCTAAAAAGGACAAAAAGAAAAAGGCTGATGAACTTCAG |
| | | GCTTTACCGCAGAGACAGAAGAAACAACAAACTGTG |
| SEQ ID NO: | 2019- | ACTATTACCAGCTGTACTCAACTCAATTGAGTACAGACACTGGTG |
| 182 | nCoV | TTGAACATGTTACCTTCTTCATCTACAATAAAATTGTTGATGAGCC |
| | E-gene | TGAAGAACATGTCCAAATTCACACAATCGACGGTTCATCCGGAGT |
| | | TGTTAATCCAGTAATGGAACCAATTTATGATGAACCGACGACGAC |
| | | TACTAGCGTGCCTTTGTAAGCACAAGCTGATGAGTACGAACTTAT |
| | | GTACTCATTCGTTTCGGAAGAGACAGGTACGTTAATAGTTAATAG |
| | | CGTACTTCTTTTTCTTGCTTTCGTGGTATTCTTGCTAGTTACACTAG |
| | | CCATCCTTACTGCGCTTCGATTGTGTGCGTACTGCTGCAATATTGT |
| | | TAACGTGAGTCTTGTAAAACCTTCTTTTTACGTTTACTCTCGTGTT |
| | | AAAAATCTGAATTCTTCTAGAGTTCCTGATCTTCTGGTCTAAACG |
| | | AACTAAATATTATATTAGTTTTTCTGTTTGGAACTTTAATTTTAGC |
| | | CATGGCAGATTCCAACGGTACTATTACCGTTGAAGAGCTTAAAAA |
| | | GCTCCTTGAACAATGGAACCTAGTAATAGGTTTCCTATTCCTTAC |
| | | ATGGATT |
| SEQ ID NO: | SARS- | TTTACTACCAGCTTGAGTCTACACAAATTACTACAGACACTGGTA |
| 183 | CoV E- | TTGAAAATGCTACATTCTTCATCTTTAACAAGCTTGTTAAAGACCC |
| | gene | ACCGAATGTGCAAATACACACAATCGACGGCTCTTCAGGAGTTGC |
| | | TAATCCAGCAATGGATCCAATTTATGATGAGCCGACGACGACTAC |
| | | TAGCGTGCCTTTGTAAGCACAAGAAAGTGAGTACGAACTTATGTA |
| | | CTCATTCGTTTCGGAAGAAACAGGTACGTTAATAGTTAATAGCGT |
| | | ACTTCTTTTTCTTGCTTTCGTGGTATTCTTGCTAGTCACACTAGCC |
| | | ATCCTTACTGCGCTTCGATTGTGTGCGTACTGCTGCAATATTGTTA |
| | | ACGTGAGTTTAGTAAAACCAACGGTTTACGTCTACTCGCGTGTTA |
| | | AAAATCTGAACTCTTCTGAAGGAGTTCCTGATCTTCTGGTCTAAA |
| | | CGAACTAACTATTATTATTATTCTGTTTGGAACTTTAACATTGCTT |
| | | ATCATGGCAGACAACGGTACTATTACCGTTGAGGAGCTTAAACAA |
| | | CTCCTGGAACAATGGAACCTAGTAATAGGTTTCCTATTCCTAGCC |
| | | TGGATT |
| SEQ ID NO: | bat-SL- | ATTACTACCAGCTGTACTCAACACAAGTGAGTACAGACACTGGTG |
| 184 | CoVZC | TTGAACATGTTACTTTCTTCATCTACAATAAAATTGTTGATGAGCC |
| | 45 E- | TGAAGAACATGTTCAAATTCACACAATCGACGGTACATCTGGAGT |
| | gene | TGTTAATCCAGCAATGGAACCAATTTATGATGAACCGACGACGAC |
| | | TACTAGCGTGCCTTTGTAAGCACAAGCTGATGAGTACGAACTTAT |
| | | GTACTCATTCGTTTCGGAAGAGACAGGTACGTTAATAGTTAATAG |
| | | CGTACTTCTTTTTCTTGCTTTTGTGGTATTCTTGCTAGTCACACTAG |
| | | CCATCCTTACTGCGCTTCGATTGTGTGCGTACTGCTGCAATATTGT |
| | | TAACGTGAGTCTTGTAAAACCTTCTTTTTACGTTTACTCTCGTGTT |
| | | AAAAATCTGAATTCTTCTAGAGTTCCTGATCTTTTGGTCTAAACGA |
| | | ACTAAATATTATATTAGTCTTTCTGTTTGGAACTTTAATTTTAGCC |
| | | ATGTCAGGTGACAACGGTACCATTACCGTTGAAGAGCTTAAAAA |
| | | GCTCTTAGAACAATGGAACCTAGTAATAGGATTCTTGTTTCTTAC |
| | | ATGGATT |

### Example 7

### Sensitivity of Detection of the E-Gene of Four Coronaviruses

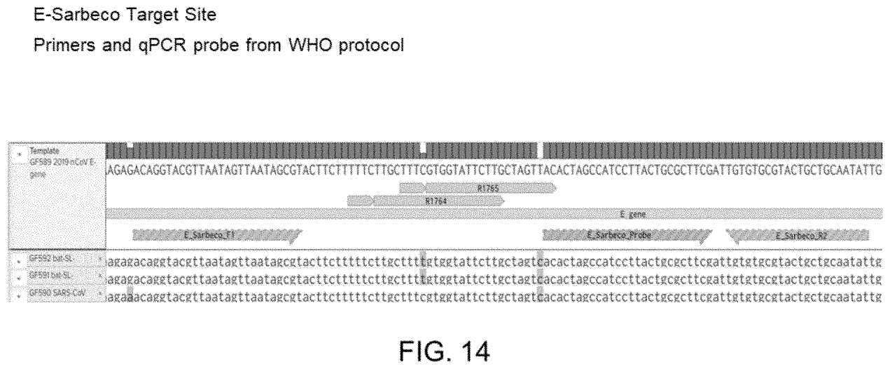

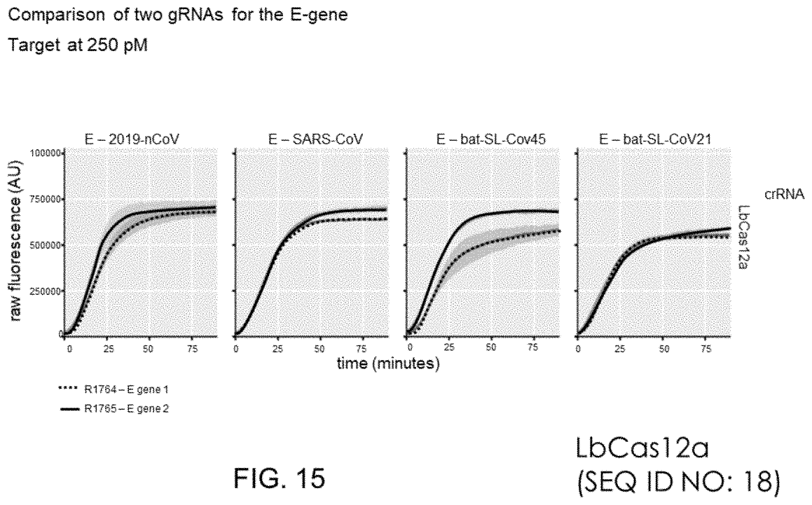

This example describes the sensitivity of detection of the E-gene of three coronaviruses. Samples containing 250 μM of either RNA corresponding to the E-gene of SARS-CoV-2, the E-gene of SARS-CoV, the E-gene of bat-SL-CoV45, or the E-gene of bat-SL-CoV21. Samples were amplified at detected as described in EXAMPLE 2. Samples were detected using each of a first gRNA directed to the E-gene (R1764), or a second gRNA directed to the E-gene (R1765). Sequences of the gRNAs used in this example are provided in TABLE 6. FIG. 14 schematically illustrates the sequence of a region of the SARS-CoV-2 E-gene (“E-Sarbeco”) target site. Target site sequences are provided in TABLE 7. FIG. 15 shows the results of a DETECTR assay to determine the sensitivity of two gRNAs directed to a coronavirus N-gene for samples containing either the E-gene of SARS-CoV-2 (“E-2019-nCoV”), the E-gene of SARS-CoV (“E-SARS-CoV”), the E-gene of bat-SL-CoV45 (“E-bat-SL-CoV45”), or the E-gene of bat-SL-CoV21 (“E-bat-SL-CoV21”). Samples were detected with LbCas12a (SEQ ID NO: 18) and either a gRNA corresponding to SEQ ID NO: 172 (“R1764-E gene 1”) or a gRNA corresponding to SEQ ID NO: 173 (“R1765-E gene 2”). Fluorescence intensity was measured over time.

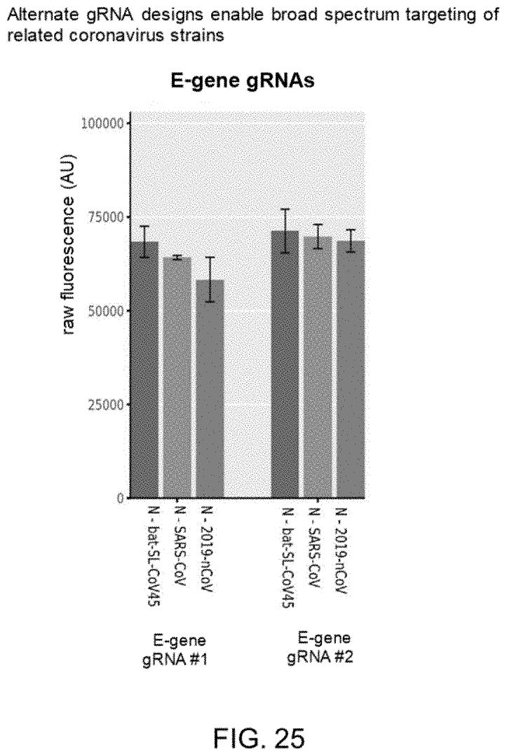

FIG. 25 shows the results of a DETECTR assay evaluating multiple gRNAs for their utility in distinguishing between three different strains of coronavirus, SARS-CoV-2 (“COVID-2019”), SARS-CoV, or bat-SL-CoV45. Samples containing E-gene amplicons of either SARS-CoV-2 (“N-2019-nCoV”), SARS-CoV (“N-SARS-CoV”), or bat-SL-CoV45 (“N-bat-SL-CoV45”) were tested. Samples were detected using gRNAs directed to the E-gene of multiple coronaviruses corresponding to SEQ ID NO: 172 (“E-gene gRNA #1”) or SEQ ID NO: 173 (“E-gene gRNA #2”). Detection of a sample with a gRNA directed to the E-gene enabled broad spectrum targeting of related coronavirus strains.

### Example 8

### Detection of a Coronavirus Using a Lateral Flow DETECTR Reaction Using a Cas12 Variant

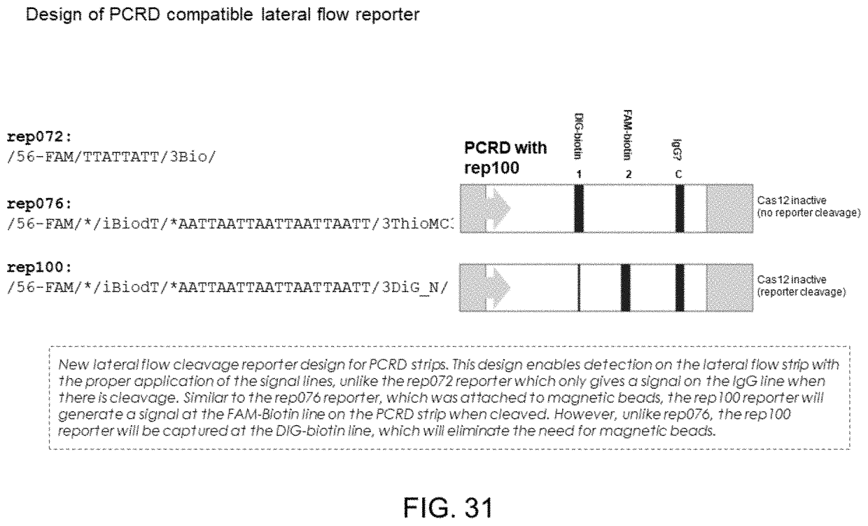

This example describes the detection of a coronavirus using a lateral flow DETECTR reaction. FIG. 31 illustrates the design of detector nucleic acids compatible with a PCRD lateral flow device. Exemplary compatible detector nucleic acids, rep072, rep076, and rep100, are provided (left). These detector nucleic acids may be used in a PCRD lateral flow device (right) to detect the presence or absence of a target nucleic acid. The top right schematic illustrates an exemplary band configuration produced when contacted to a sample that does not contain a target nucleic acid. The bottom right schematic shows an exemplary band configuration produced when contacted to a sample that does contain a target nucleic acid. Exemplary reporters compatible with a PCRD lateral flow device are provided in TABLE 8. The lateral flow cleavage reporter Rep100 enables detection of a sample on a lateral flow strip with application of the signal lines. The Rep072 reporter only gives a signal on the IgG line following cleavage of the reporter by a programmable nuclease. Similar to the rep076 reporter, which is attached to magnetic beads, the rep100 reporter generates a signal at the FAM-Biotin line on the PCRD strip when cleaved. However, unlike rep076, the rep100 reporter is captured at the DIG-biotin line, which eliminates the need for magnetic beads.

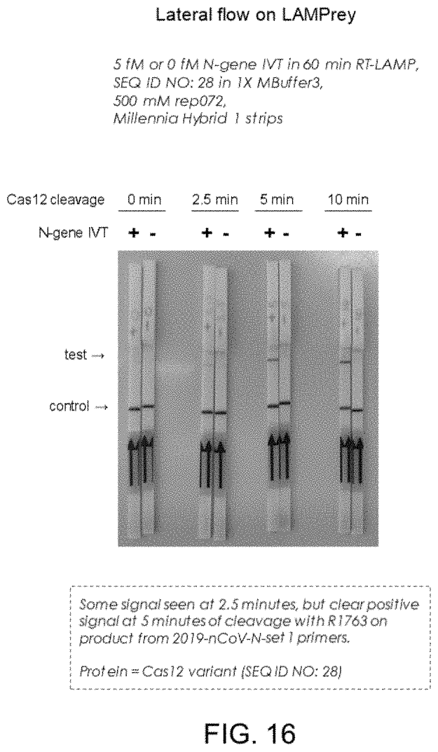

A sample comprising an RNA target sequence from a coronavirus was amplified using isothermal amplification. Samples containing either 0 fM (“−”) or 5 fM (“+”) of in vitro transcribed coronavirus N-gene were amplified for 60 minutes using a reverse-transcription LAMP (RT-LAMP) amplification assay. A DETECTR reaction was performed using a Cas12 variant (SEQ ID NO: 28) for either 0 min, 2.5 min, 5 min, or 10 min. FIG. 16 shows the results of a lateral flow DETECTR reaction to detect the presence or absence of a SARS-CoV-2 N-gene target RNA using a Cas12 variant (SEQ ID NO: 28). Lateral flow test strips are shown. Samples either containing (“+”) or lacking (“−”) in vitro transcribed SARS-CoV-2 N-gene RNA (“N-gene IVT”) were tested. The top set of horizontal lines (denoted “test”) indicated the results of the DETECTR reaction. The DETECTR reaction was sensitive for samples containing the in vitro transcribed coronavirus target sequence.

TABLE 8
Exemplary Reporter Sequences for Detection of
Coronaviruses

| Reporter | Sequence |
|---|---|
| Rep072 | /56-FAM/TTATTATT/3Bio/ |
| Rep076 | /56-FAM/*/iBiodT/*AATTAATTAATTAATTAATT/ |
| | 3ThioMC3-D/ (SEQ ID NO: 185) |
| Rep100 | /56-FAM/*/iBiodT/* AATTAATTAATTAATTAATT/ |
| | 3DiG_N/ (SEQ ID NO: 185) |

### Example 9

### Detection of SARS-CoV-2 Using a Lateral Flow DETECTR Reaction

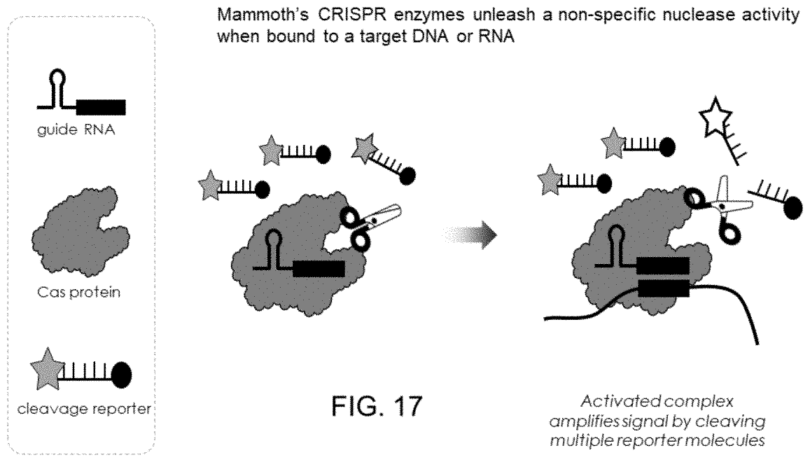

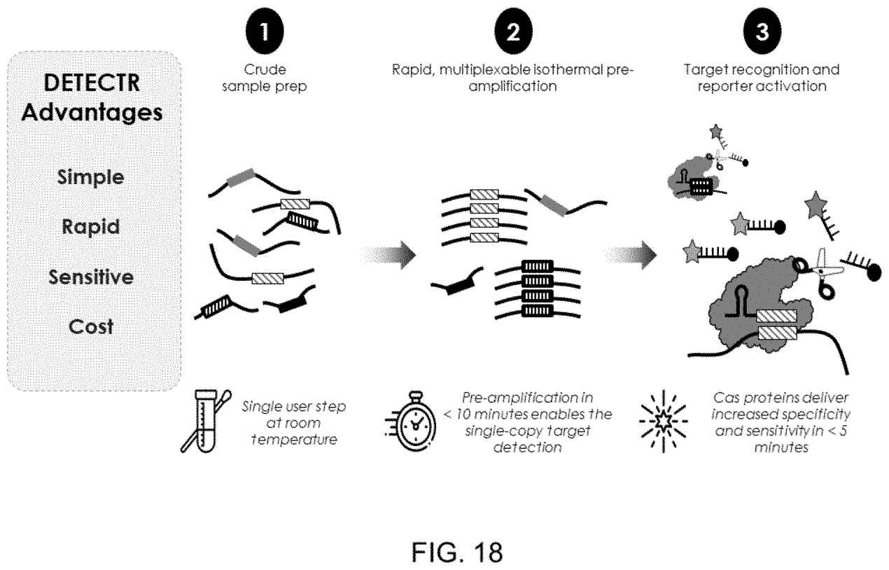

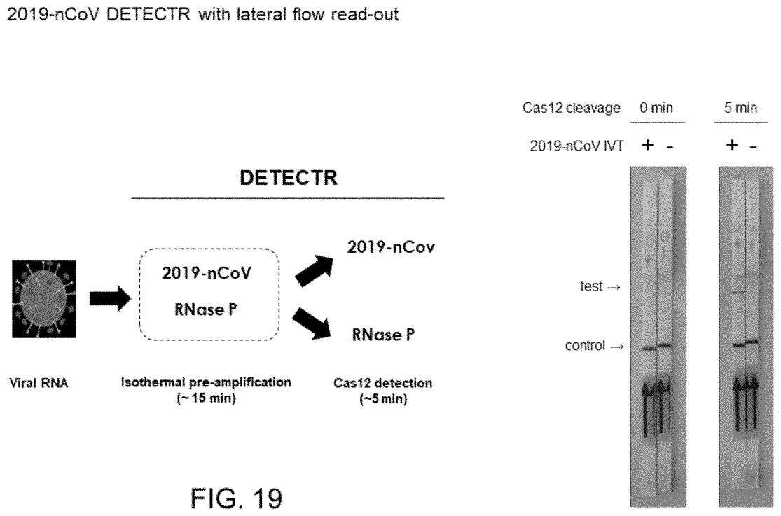

This example describes the detection of SARS-CoV-2 using a lateral flow DETECTR reaction. FIG. 17 illustrates schematically the detection of a target nucleic acid using a programmable nuclease. Briefly, a Cas protein with trans collateral cleavage activity is activated upon binding to a guide nucleic acid and a target sequence reverse complementary to a region of the guide nucleic acid. The activated programmable nuclease cleaves a reporter nucleic acid, thereby producing a detectable signal. FIG. 18 illustrates schematically detection of the presence or absence of a target nucleic acid in a sample. Select nucleic acids in a sample are amplified using isothermal amplification. The amplified sample is contacted to a programmable nuclease, a guide nucleic acid, and a reporter nucleic acid, as illustrated in FIG. 17. If the sample contains the target nucleic acid, a detectable signal is produced. The presence or absence of a target nucleic acid corresponding to SARS-CoV-2 was detected using a DETECTR reaction following in vitro transcription and isothermal pre-amplification of the target nucleic acid. Samples were detected using a Cas12 programmable nuclease. Samples contained either SARS-CoV-2 viral RNA or a sequence corresponding to RNase P (negative control). Samples were detected using a gRNA directed to SARS-CoV-2 using the DETECTR reaction described in FIG. 17 and FIG. 18. FIG. 19 shows the results of a DETECTR lateral flow reaction to detect the presence or absence of SARS-CoV-2 (“2019-nCoV”) RNA in a sample. Detection of RNase P is used as a sample quality control. Samples were in vitro transcribed and amplified (left) and detected using a Cas12 programmable nuclease (right). Samples containing (“+”) or lacking (“−”) in vitro transcribed SARS-CoV-2 RNA (“2019-nCoV IVT”) were assayed with a Cas12 programmable nuclease and gRNA directed to SARS-CoV-2 for either 0 min or 5 min. The reaction was sensitive for samples containing SARS-CoV-2.

### Example 10

### Testing Clinical Samples for SARS-CoV-2 Using a DETECTR Reaction

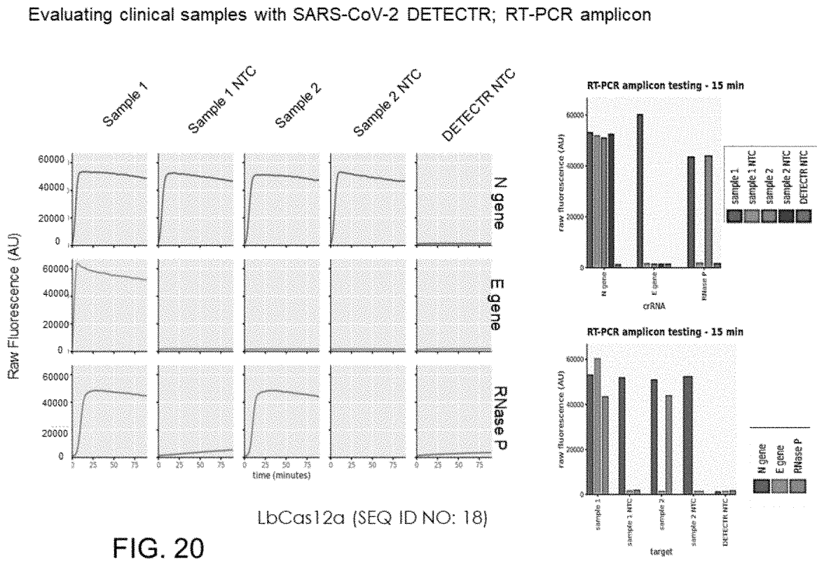

This example describes the testing of clinical samples for SARS-CoV-2 using a DETECTR reaction. Clinical samples were amplified using RT-PCR and detected using LbCas12a. Samples were detected using gRNA (“crRNA”) directed to either the N-gene or the E-gene of SARS-CoV-2 or RNase P (negative control). FIG. 20 shows the results of a DETECTR reaction using an LbCas12a programmable nuclease (SEQ ID NO: 18) to determine the presence or absence of SARS-CoV-2 in patient samples.

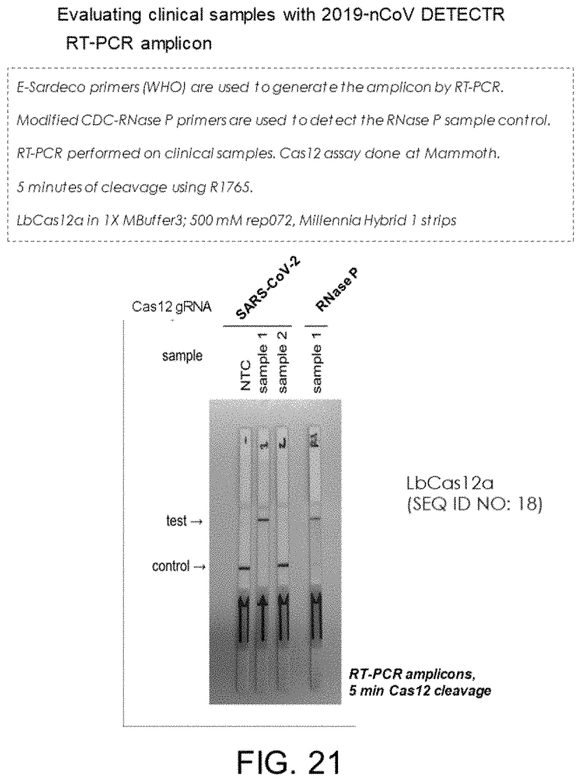

Clinical samples of patients either positive or negative for SARS-CoV-2 were assayed using a lateral flow DETECTR reaction. Samples were amplified and reverse transcribed using RT-PCR and detected using a Cas12 programmable nuclease. A negative control sample (“NTC”) was also assayed. The DETECTR reaction was performed for 5 min. FIG. 21 shows the results of a lateral flow DETECTR reaction to detect the presence or absence of SARS-CoV-2 in patient samples. Samples were detected with either a gRNA directed to SARS-CoV-2 or a gRNA directed to RNase P. Primers directed to a region of the E-gene were used to amplify the target region using RT-PCR.

### Example 11

### Buffer Screening for Improved RT-LAMP Amplification and Detection

This example describes buffer screening for improved RT-LAMP amplification and detection. Samples containing either HeLa total RNA (“total RNA”), SARS-CoV-2 N-gene RNA and HeLa total RNA (“N-gene+total RNA”) or no target (“NTC”) were amplified using RT-LAMP under different buffer conditions.

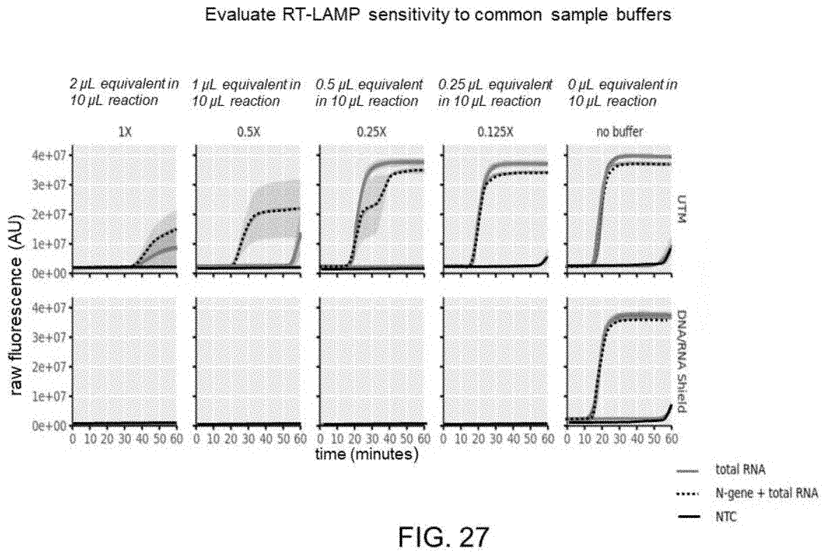

FIG. 27 shows the results of a DETECTR assay evaluating the sensitivity of an RT-LAMP amplification reaction to common sample buffers. Reactions were measured in universal transport medium (UTM, top) or DNA/RNA Shield buffer (bottom) at different buffer dilutions (from left to right: 1×, 0.5×, 0.25×, 0.125×, or no buffer).

### Example 12

### Limit of Detection of SARS-CoV-2 in a DETECTR Assay

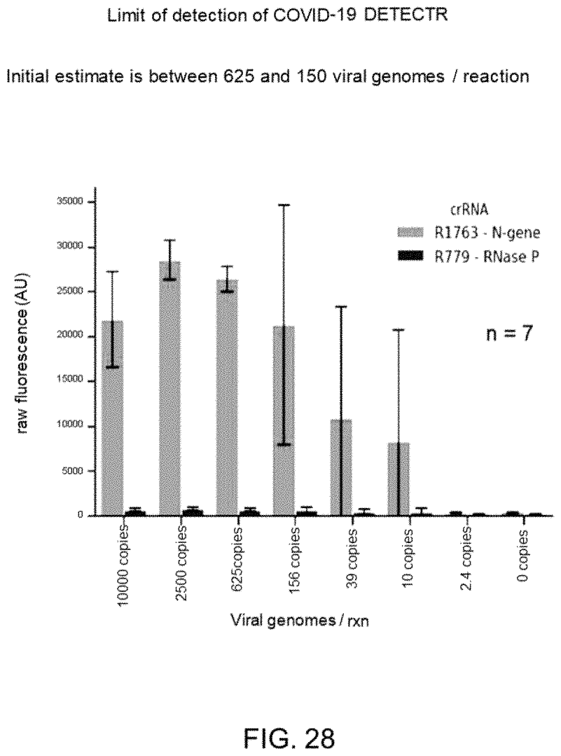

This example describes the limit of detection of SARS-CoV-2 in a DETECTR assay. DETECTR reactions were performed with different copy numbers of SARS-CoV-2 viral genomes. FIG. 28 shows the results of a DETECTR assay to determine the limit of detection (LoD) of the DETECTR assay for SARS-CoV-2 (the virus attributed to the COVID-19 infection). Samples were detected using either a gRNA directed to the N-gene of SARS-CoV-2 (SEQ ID NO: 171, “R1763-N-gene”) or a gRNA directed to RNase P (SEQ ID NO: 178, “R779-RNase P”). Each condition was repeated 7 times. The DETECTR assay was capable of reproducibly and specifically detecting the presence of SARS-CoV-2 RNA down to between about 625 and about 150 copies per reaction.

### Example 13

### Target Specificity of a Multiplexed RT-LAMP Amplification with DETECTR

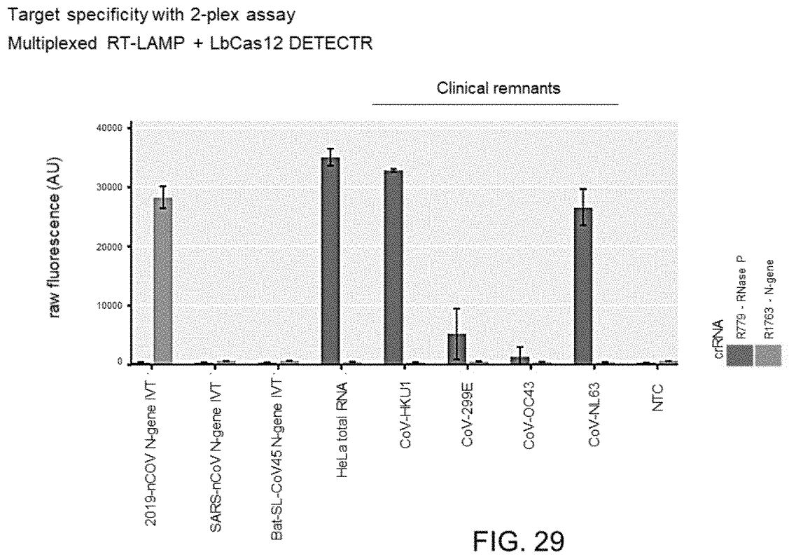

This example describes the target specificity of a multiplexed RT-LAMP amplification with DETECTR reaction. FIG. 29 shows the results of a DETECTR assay evaluating the target specificity of a gRNA directed to the N-gene of SARS-CoV-2 (“R1763-N-gene”) in a 2-plex multiplexed RT-LAMP reaction using an LbCas12a programmable nuclease (SEQ ID NO: 18). In vitro transcribed coronavirus N-gene sequences from either SARS-CoV-2 (“2019-nCoV N-gene IVT), SARS-CoV (“SARS-CoV N-gene IVT”), or bat-SL-CoV45 (“bat-SL-CoV45 N-gene IVT”) or clinical remnant samples from patients having different strains of coronavirus (CoV-HKU1, CoV-299E, CoV-OC43, or CoV-NL63) were amplified using a 2-plex multiplexed RT-LAMP amplification. HeLa total RNA was used as a positive control for RNase P. A no target control (“NTC”) was tested as a negative control. The 2-plex multiplexed RT-LAMP amplification amplified the samples using two primer sets, one directed to the SARS-CoV-2 N-gene and one directed to RNase P. Amplified samples were detected using either a gRNA directed to RNase P (SEQ ID NO: 178, “R779-RNase P”) or the N-gene of SARS-CoV-2 (SEQ ID NO: 171, “R1763-N-gene”). Both gRNAs were capable of detecting samples amplified in a 2-plex multiplexed RT-LAMP amplification assay.

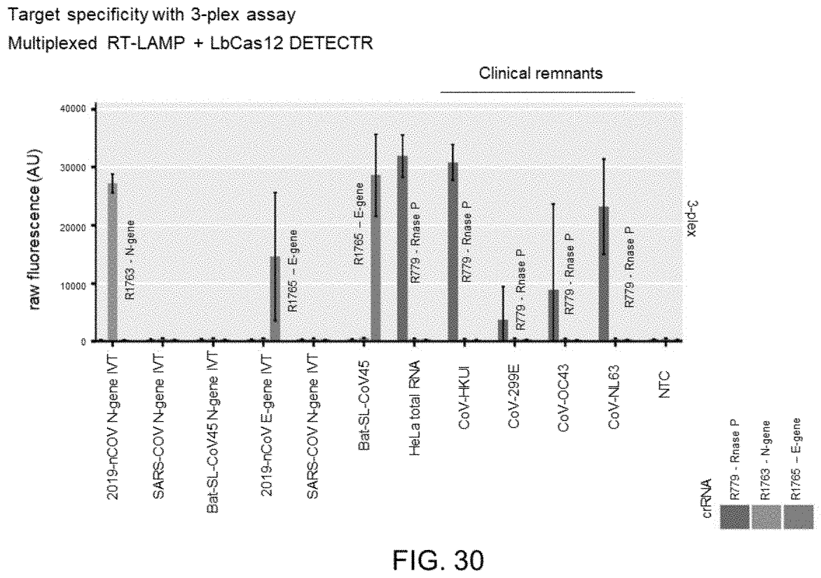

FIG. 30 shows the results of a DETECTR assay evaluating the target specificity of a gRNA directed to the N-gene of SARS-CoV-2 (“R1763-N-gene”) or the E-gene of SARS-CoV-2 (“R1765-E-gene”) in a 3-plex multiplexed RT-LAMP reaction using an LbCas12a programmable nuclease (SEQ ID NO: 18). In vitro transcribed coronavirus N-gene sequences from either SARS-CoV-2 (“2019-nCoV N-gene IVT), SARS-CoV (“SARS-CoV N-gene IVT”), or bat-SL-CoV45 (“bat-SL-CoV45 N-gene IVT”), in vitro transcribed coronavirus E-gene sequences from SARS-CoV-2 (“2019-nCoV E-gene IVT) or SARS-CoV (“SARS-CoV E-gene IVT”), or clinical remnant samples from patients having different strains of coronavirus (CoV-HKU1, CoV-299E, CoV-OC43, or CoV-NL63) were amplified using a 3-plex multiplexed RT-LAMP amplification. HeLa total RNA was used as a positive control for RNase P. A no target control (“NTC”) was tested as a negative control. The 3-plex multiplexed RT-LAMP amplification amplified the samples using three primer sets, one directed to the SARS-CoV-2 N-gene, one directed to the SARS-CoV-2 E-gene, and one directed to RNase P. Amplified samples were detected using either a gRNA directed to RNase P (SEQ ID NO: 178, “R779-RNase P”), the N-gene of SARS-CoV-2 (SEQ ID NO: 171, “R1763-N-gene”), or the E-gene of SARS-CoV-2 (SEQ ID NO: 173, “R1765-E-gene”). All three gRNAs were capable of detecting samples amplified in a 3-plex multiplexed RT-LAMP amplification assay.

### Example 14

### Coronavirus Strain Specificity of N-Gene and E-Gene gRNAs

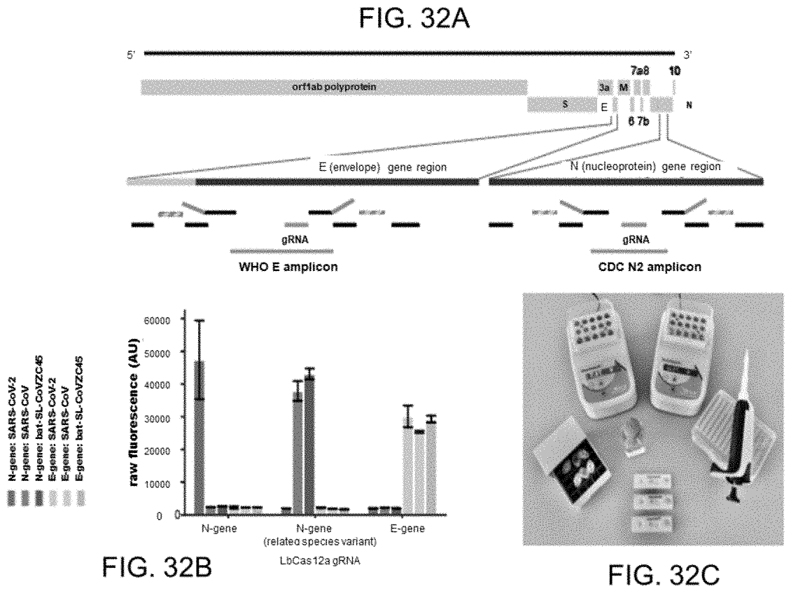

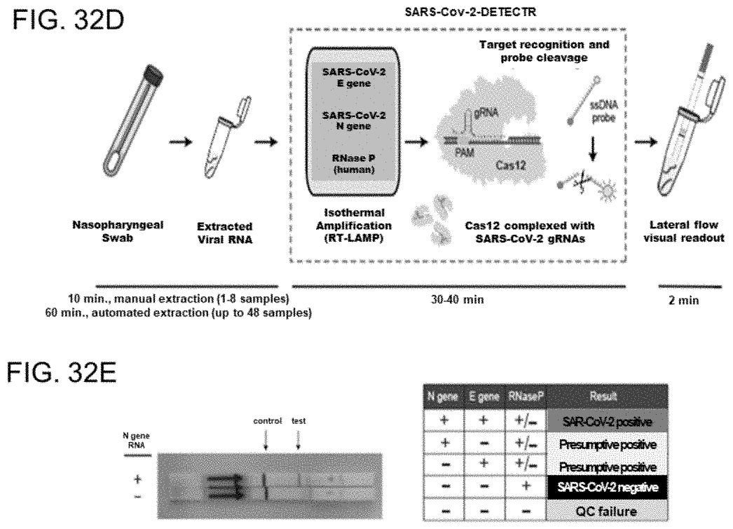

This example describes coronavirus strain specificity of N-gene and E-gene gRNAs. Guide RNAs were designed to specifically detect the N-gene of SARS-CoV-2. Guide RNAs were also designed to detect the E-gene in three SARS-like coronavirus strains (SARS-CoV, bat SARS-like coronavirus (bat-SL-CoVZC45), and SARS-CoV-2). Synthetic in vitro transcribed (IVT) SARS-CoV-2 RNA gene targets were spiked into nuclease-free water. Samples were detected with a CRISPR-Cas12 based detection assay using LbCas12a (SEQ ID NO: 18). DETECTR assays included an RT-LAMP reaction at 62° C. for 20 min and Cas12 detection reaction at 37° C. for 10 min. Primers for target generation, qPCR, and LAMP amplification are provided in TABLE 9. FIG. 32A illustrates a genome map indicating the locations of the E (envelope) gene and the N (nucleoprotein) gene regions within a coronavirus genome. Corresponding regions or annealing regions of primers and probes relative to the E and N gene regions are shown below the respective gene regions. RT-LAMP primers are indicated by black rectangles, the binding position of the F1c and B1c half of the FIP primer (grey) is represented by a striped rectangle with dashed borders. Regions amplified in tests utilized by the World Health Organization (WHO) and the Center for Disease Control (CDC) are denoted as “WHO E amplicon” and “CDC N2 amplicon,” respectively.

Guide RNAs were able to distinguish SARS-CoV-2 without cross-reactivity with related coronavirus strains using the N gene gRNA and with the expected cross-reactivity for the E gene gRNA. FIG. 32B shows the results of a DETECTR assay evaluating the specificity or broad detection utility of gRNAs directed to the N-gene or E-gene of various coronavirus strains (SARS-CoV-2, SARS-CoV, or bat-SL-CoVZC45) using an LbCas12a programmable nuclease (SEQ ID NO: 18). The N gene gRNA used in the assay (left, “N-gene”) was specific for SARS-CoV-2, whereas the E gene gRNA was able to detect 3 SARS-like coronavirus (right, “E-gene”). A separate N gene gRNA targeting SARS-CoV and a bat coronavirus failed to detect SARS-CoV-2 (middle, “N-gene related species variant”). Guide RNAs were designed to specifically target SARS-CoV-2 or broadly detect related coronavirus strains. Samples containing either SARS-CoV-2 N-gene (“N-gene: SARS-CoV-2”), SARS-CoV N-gene (“N-gene: SARS-CoV”), bat-SL-CoVZC45 N-gene (“N-gene: bat-SL-CoVZC45”), SARS-CoV-2 E-gene (“E-gene: SARS-CoV-2”), SARS-CoV E-gene (“E-gene: SARS-CoV”), or bat-SL-CoVZC45 E-gene (“E-gene: bat-SL-CoVZC45”) were detected using either a gRNA designed to specifically detect the SARS-CoV N-gene (SEQ ID NO: 171, “N-gene”), a gRNA designed to detect the N-gene of coronavirus variants (SEQ ID NO: 174, “N-gene (related species variant)”), or a gRNA designed to broadly detect coronavirus E-gene (SEQ ID NO: 172, “E-gene”).

TABLE 9
Target Generation and Amplification Primers

| SEQ ID NO: | Name | Sequence | Purpose |
|---|---|---|---|
| SEQ ID NO: 186 | N-gene-FWD IVT | AATTCTAATACGACTCACTATAGGGCC | Target |
| | | AAATTGGCTACTACCGAAGAGCTAC | Generation |
| SEQ ID NO: 187 | N-gene-REV IVT | CACAGTTTGCTGTTTCTTCTGTCTCTGC | Target |
| | | GG | Generation |
| SEQ ID NO: 188 | E-gene-FWD IVT | AATTCTAATACGACTCACTATAGGGCT | Target |
| | | GGTGTTGAACATGTTACCTTCTTCATC | Generation |
| SEQ ID NO: 189 | E-gene-REV IVT | CCTATTACTAGGTTCCATTGTTC | Target |
| | | | Generation |
| SEQ ID NO: 190 | E_Sarbeco_F1 | ACAGGTACGTTAATAGTTAATAGCGT | qPCR |
| SEQ ID NO: 191 | E_Sarbeco_R2 | ATATTGCAGCAGTACGCACACA | qPCR |
| SEQ ID NO: 192 | CDC N2-FWD | TTACAAACATTGGCCGCAAA | qPCR |
| SEQ ID NO: 193 | CDC N2-REV | GCGCGACATTCCGAAGAA | qPCR |
| SEQ ID NO: 194 | F3 2019-nCoV N- | AACACAAGCTTTCGGCAG | LAMP |
| | gene | | |
| SEQ ID NO: 195 | B3 2019-nCoV N- | GAAATTTGGATCTTTGTCATCC | LAMP |
| | gene | | |
| SEQ ID NO: 196 | BIP 2019-nCoV | TGCGGCCAATGTTTGTAATCAGCCAAG | LAMP |
| | N-gene | GAAATTTTGGGGAC | |
| SEQ ID NO: 197 | FIP 2019-nCoV | CGCATTGGCATGGAAGTCACTTTGATG | LAMP |
| | N-gene | GCACCTGTGTAG | |
| SEQ ID NO: 198 | LF 2019-nCoV N- | TTCCTTGTCTGATTAGTTC | LAMP |
| | gene | | |
| SEQ ID NO: 199 | LB 2019-nCoV N- | ACCTTCGGGAACGTGGTT | LAMP |
| | gene | | |
| SEQ ID NO: 200 | F3 2019-nCoV E- | CCGACGACGACTACTAGC | LAMP |
| | gene | | |
| SEQ ID NO: 201 | B3 2019-nCoV E- | AGAGTAAACGTAAAAAGAAGGTT | LAMP |
| | gene | | |
| SEQ ID NO: 202 | BIP 2019-nCoV | ACCTGTCTCTTCCGAAACGAATTTGTA | LAMP |
| | E-gene | AGCACAAGCTGATG | |
| SEQ ID NO: 203 | FIP 2019-nCoV E- | CTAGCCATCCTTACTGCGCTACTCACG | LAMP |
| | gene | TTAACAATATTGCA | |
| SEQ ID NO: 204 | LF 2019-nCoV E- | TCGATTGTGTGCGTACTGC | LAMP |
| | gene | | |
| SEQ ID NO: 205 | LB 2019-nCoV E- | TGAGTACATAAGTTCGTAC | LAMP |
| | gene | | |
| SEQ ID NO: 206 | F3 RNase P POP7 | TTGATGAGCTGGAGCCA | LAMP |
| SEQ ID NO: 207 | B3 RNase P POP7 | CACCCTCAATGCAGAGTC | LAMP |
| SEQ ID NO: 208 | FIP RNase P | GTGTGACCCTGAAGACTCGGTTTTAGC | LAMP |
| | POP7 | CACTGACTCGGATC | |
| SEQ ID NO: 209 | BIP RNase P | CCTCCGTGATATGGCTCTTCGTTTTTTT | LAMP |
| | POP7 | CTTACATGGCTCTGGTC | |
| SEQ ID NO: 210 | LF RNase P POP7 | ATGTGGATGGCTGAGTTGTT | LAMP |
| SEQ ID NO: 211 | LB RNase P POP7 | CATGCTGAGTACTGGACCTC | LAMP |
| SEQ ID NO: 212 | F3 RNase P POP7 | CACATCCGAGTCTTCAGG | LAMP |
| | v2 | | |
| SEQ ID NO: 213 | B3 RNase P POP7 | GGCAATAGTTACAGACCGC | LAMP |
| | v2 | | |
| SEQ ID NO: 214 | FIP RNase P | TCCAGTACTCAGCATGCGAAGCACCCA | LAMP |
| | POP7 v2 | AGTAATTGAAAAGACAC | |
| SEQ ID NO: 215 | BIP RNase P | CTGGAAGCCCAAAGGACTCTATACACA | LAMP |
| | POP7 v2 | CACTCAGGAAGG | |
| SEQ ID NO: 216 | LF RNase P POP7 | CGGAGGGGATAAGTGGAGGA | LAMP |
| | v2 | | |
| SEQ ID NO: 217 | LB RNase P POP7 | GCATTGAGGGTGGGGGT | LAMP |
| | v2 | | |

### Example 15

### Specific and Broad Detection of Coronaviruses Using a Lateral Flow DETECTR Assay

This example describes specific and broad detection of coronaviruses using a lateral flow DETECTR assay. Lateral flow DETECTR assays can be performed with minimal equipment within appropriate biosafety laboratory requirements. FIG. 32C shows exemplary laboratory equipment utilized in the coronavirus lateral flow DETECTR assays. In addition to appropriate biosafety protective equipment, the equipment utilized includes a sample collection device, microcentrifuge tubes, heat blocks set to 37° C. and 62° C., pipettes and tips, and lateral flow strips.

The DETECTR assay can be run within 30 to 40 minutes and visualized on a lateral flow strip. Conventional RNA extraction or sample matrix can be used as an input to DETECTR (LAMP pre-amplification and Cas12-based detection for N gene, E gene and RNase P), which is visualized by a fluorescent reader or lateral flow strip. The SARS-CoV-2 DETECTR assay was considered positive if there was detection of both the E and N genes, or presumptive positive if there was detection of either the E or N gene. This interpretation is consistent with that of current FDA Emergency Use Authorization (EUA) guidance and recently approved point-of-care diagnostics under the EUA. FIG. 32D illustrates an exemplary workflow of a DETECTR assay for the detection of a coronavirus in a subject. Patient samples are collected using a nasopharyngeal swab. Conventional RNA extraction or sample matrix can be used as an input to DETECTR (LAMP pre-amplification and Cas12-based detection for NE gene, EN gene and RNase P), which is visualized by a fluorescent reader or lateral flow strip. Samples can be detected directly from the raw sample matrix, or the viral RNA can be extracted and then detected. Viral RNA encoding SARS-CoV-2 E-gene and SARS-CoV N-gene and RNA encoding human RNase P is amplified using an isothermal amplification method such as RT-LAMP. Amplified samples are detected using a Cas12 programmable nuclease complexed with gRNAs directed to SARS-CoV-2 N-gene and E-gene sequences. The Cas12 programmable nuclease cleaves a ssDNA reporter nucleic acid upon complex formation with the target nucleic acid. The sample is then detected using a lateral flow readout. Sample collection may be performed in about 0 min to about 10 min, amplification and detection may be performed in about 20 min to about 30 min, and sample readout may be performed in about 2 min.

FIG. 32E shows lateral flow test strips (left) indicating a positive test result for SARS-CoV-2 N-gene (left, top) and a negative test result for SARS-CoV-2 N-gene (left, bottom). A positive identification SARS-CoV-2 in a sample required detection of both the E-gene and the N-gene to confirm a positive test. The lateral assay was performed as illustrated and described in FIG. 32D. The table (right) illustrates possible test indicators and associated results for a lateral flow strip-based coronavirus diagnostic assay that tests for the presences of absence of the RNase P (positive control), SARS-CoV-2 N-gene, and coronavirus E-gene. Detection of the two SARS-CoV-2 viral gene targets and the internal spiked human RNase P control indicates a positive result.

### Example 16

### Amplification and Detection of Patient Samples Directly from Raw Sample Matrix

This example describes amplification and detection of patient samples directly from raw sample matrix. The capability of the RT-LAMP assay to amplify SARS-CoV-2 nucleic acid directly from raw sample matrix was assessed. Samples consisting of nasal swabs from asymptomatic donors placed in universal transport medium (UTM) or phosphate buffered saline (PBS) and spiked with SARS-CoV-2 IVT target RNA were assayed using RT-LAMP DETECTR reactions. Since nasal swabs are more frequently collected in universal transport medium (UTM) than in phosphate buffered saline (PBS), the effect of running the assay from nasal swab sample matrix consisting of UTM buffer was evaluated. Nasal swabs from asymptomatic donors were collected in UTM or PBS.

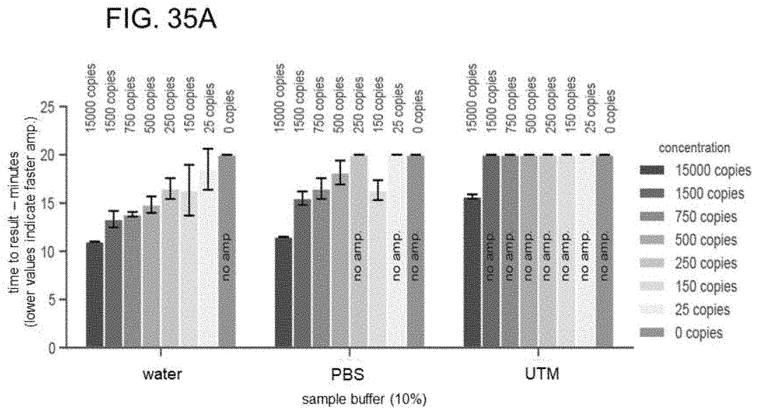

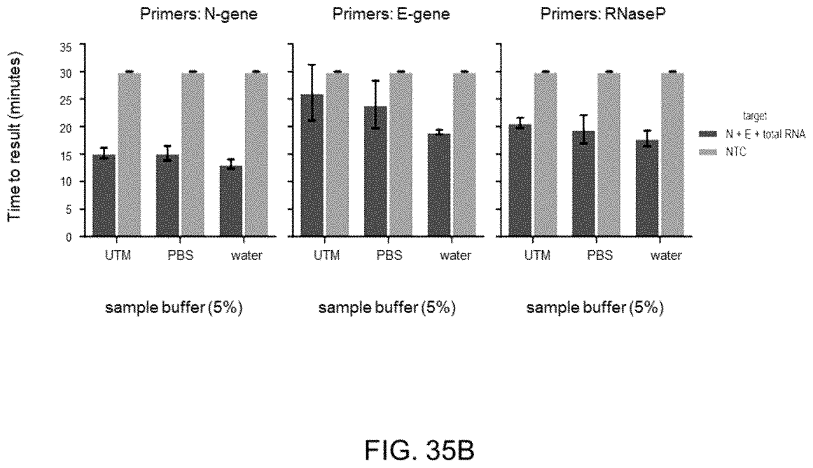

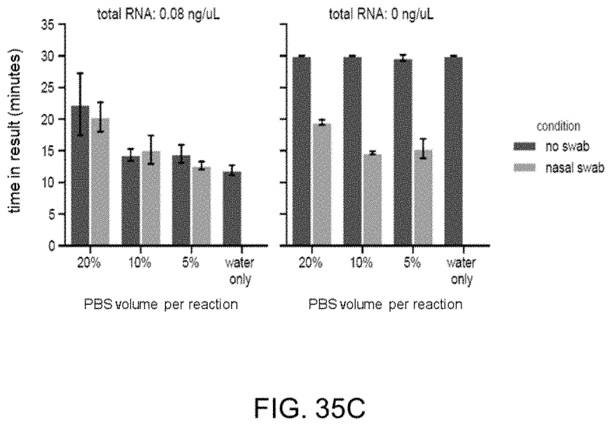

FIG. 35A shows the time to result of an RT-LAMP amplification under different buffer conditions. Time to results was calculated as the time at which the fluorescent value is one third of the max for the experiment. Reactions that failed to amplify are reported with a value of 20 minutes and labeled as “no amp.” Time to result was determined for different starting concentrations of target control plasmid in either water, 10% phosphate buffered saline (PBS), or 10% universal transport medium (UTM). A lower time to result indicates faster amplification. Results indicate that 10% PBS inhibits the assay less than 10% UTM. FIG. 35B shows the results of an RT-LAMP assay to determine the amplification efficiency of the N-gene of SARS-CoV-2, the E-gene of SARS-CoV-2, and RNase P in either 5% UTM, 5% PBS, or water. Samples containing 0.5 fM N-gene in vitro transcribed, 0.5 fM of E-gene in vitro transcribed, and 0.8 ng/μL HeLa total RNA (“N+E+total RNA”) or no target controls (“NTC”) were tested. Evaluation of amplification efficiency for RT-LAMP for the N-gene, E-gene, and RNase P in 5% sample buffer final volume showed that RT-LAMP was functional for all target genes at a 5% sample buffer concentration. Final target concentrations were at 0.5 fM N-gene IVT, 0.5 fM E-gene IVT, and 0.08 ng/μL HeLa total RNA. FIG. 35C shows amplification of RNA directly from nasal swabs in PBS. Time to result was measured as a function of PBS concentration. Nasal swabs (“nasal swab”) were either spiked with HeLa total RNA (left, “total RNA: 0.08 ng/uL”) or water (right, “total RNA: 0 ng/uL”). Samples without a nasal swab (“no swab”) were compared as controls. With RT-LAMP, assay performance degraded at reaction concentrations of ≥10% UTM by volume or ≥20% PBS by volume. The estimated limit of detection decreased to 500 copies/μL in ≥10% UTM and to 1,00 copies/μL in ≥20% PBS. RT-LAMP was capable of amplifying RNA directly from nasal swabs in PBS with the best performance at 5% or 10% final volume of PBS per RT-LAMP pre-amplification reaction. Nasal swabs were prepared in PBS and either spiked with HeLa total RNA or water and run at various concentrations in an RT-LAMP reaction for RNase P.

### Example 17

### Limit of Detection of a DETECTR Assay for SARS-CoV-2

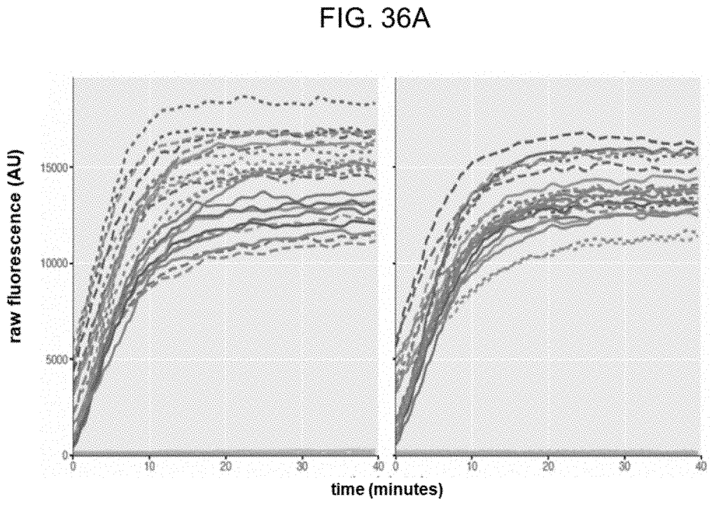

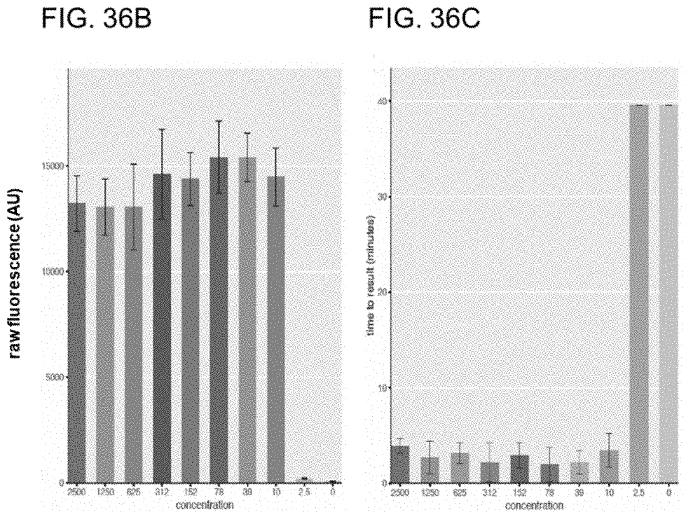

This example describes the limit of detection of a DETECTR assay for SARS-CoV-2. Using IVT SARS-CoV-2 target RNA spiked into donor nasal swab sample matrix in PBS, the analytic limits of detection (LoD) of the DETECTR assay was compared relative to the US FDA Emergency Use Authorization (EUA)-approved CDC assay (running tests for 2 of the 3 targets, N2 and N3) for detection of SARS-CoV-2. Five 10-fold serial dilutions of in vitro transcribed viral RNA were spiked into sample matrix at concentrations ranging from 101-105 copies/mL, with 6 replicates at each dilution for the DETECTR assay, and 3 replicates at each dilution for the CDC assay. FIG. 36A shows raw fluorescence curves generated by LbCas12a (SEQ ID NO: 18) detection of SARS-CoV-2 N-gene (n=6). The curves showed saturation in less than 20 minutes. FIG. 36B shows the limit of detection of a DETECTR assay for the SARS-CoV-2 N-gene detected with LbCas12a, as determined from the raw fluorescence traces shown in FIG. 36A. Fluorescence intensity was measured with decreasing concentration (copies per mL) of SARS-CoV-2 N-gene. FIG. 36C shows the time to result of the limit of detection DETECTR assay, as determined from the raw fluorescence traces shown in FIG. 36A. A lower time to result indicated faster amplification and detection. The estimated LoD for SARS-CoV-2 DETECTR was approximately 10 copies/μl, which is comparable to the LoD for the CDC N2 and N3 assays. DETECTR analysis of SARS-CoV-2 identified down to 10 viral genomes in less than 30 minutes. Duplicate LAMP reactions were amplified for twenty minutes followed by LbCas12a DETECTR analysis. Further analysis reveals the limit of detection of the SARS-CoV-2 N-gene to be 10 viral genomes per reaction (n=6, FIG. 36B). Evaluation of the time to result of these reactions highlights detection of 10 viral genomes of SARS-CoV-2 in under 5 minutes (n=6, FIG. 36C).

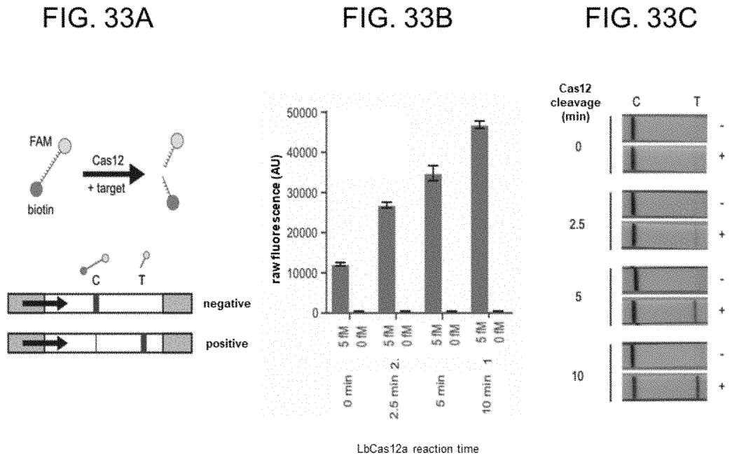

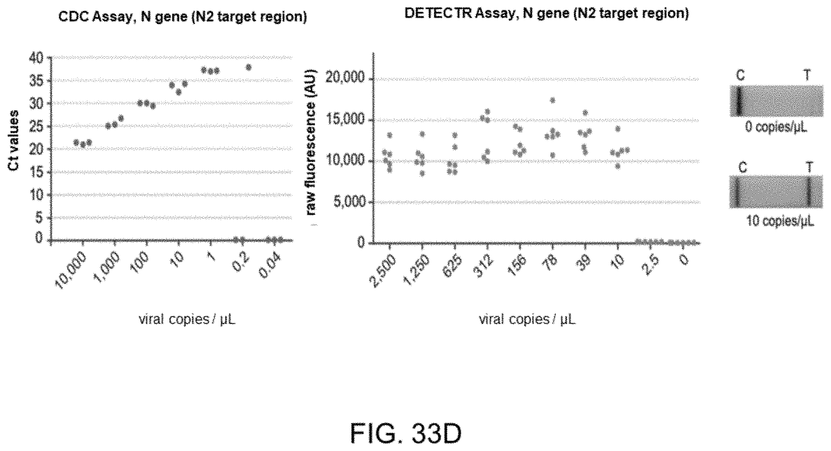

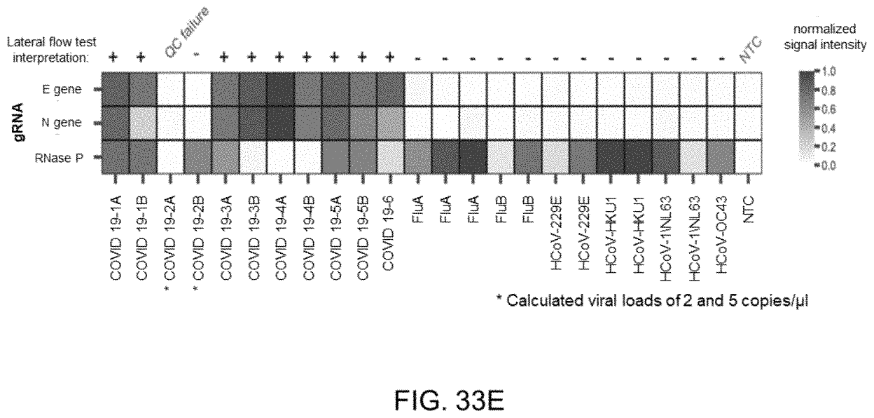

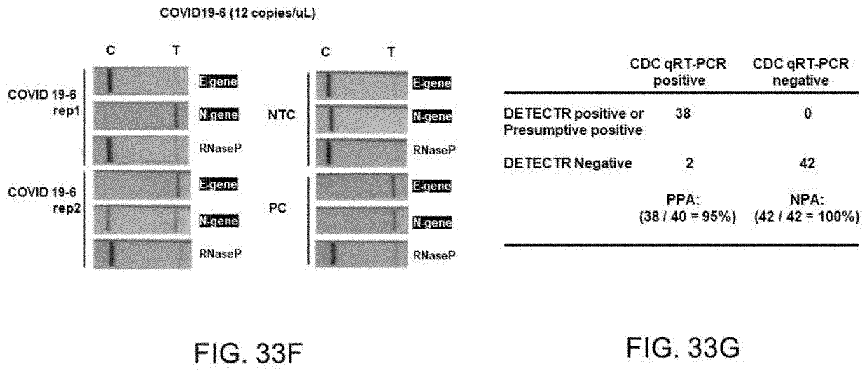

The analytic limit of detection of the RT-LAMP DETECTR reaction was compared relative to the qRT-PCR detection assay used by the US FDA Emergency Use Authorization-approved CDC assay for detection of SARS-CoV-2. A standard curve for quantitation was constructed using 7 dilutions of a control IVT viral nucleoprotein RNA (“CDC VTC nCoV Transcript”), with 3 replicates at each dilution, and detected using the CDC protocol (FIG. 33D, left). Ten two-fold serial dilutions of the same control nucleoprotein RNA were then used to run the DETECTR assay, with 6 replicates at each dilution (FIG. 33D, middle). The estimated limit of dilution for the CDC assay tested by California Department of Public Health was 1 copy/μL reaction, consistent with the analytic performance in the FDA package insert, versus 10 copies/μL reaction for the DETECTR assay. FIG. 33D shows the results of a DETECTR assay with LbCas12a (middle) or a CDC protocol (left) to determine the limit of detection of SARS-CoV-2. Signal is shown as a function of the number of copies of viral genome per reaction. Representative lateral flow results for the assay shown for 0 copies/μL and 10 copies/μL (right).

The limit of detection (LoD) was measured for detection of SARS-CoV-2 using a lateral flow device. FIG. 33A illustrates cleavage of a detector nucleic acid labeled with FAM and biotin by a Cas12 programmable nuclease in the presence of a target nucleic acid (top). Schematics of lateral flow test strips (bottom) illustrate markings indicative of either the presence (“positive”) or absence (“negative”) of the target nucleic acid in the tested sample. The intact FAM-biotinylated reporter molecule flows to the control capture line. Upon recognition of the matching target, the Cas-gRNA complex cleaves the reporter molecule, which flows to the target capture line.

### Example 18

### Effects of Incubation Time in a DETECTR Assay for SARS-CoV-2

This example describes the effects of incubation time in a DETECTR assay for SARS-CoV-2. Samples were amplified using RT-LAMP and detected using LbCas12a (SEQ ID NO: 18). The effect of the Cas12 reaction incubation time on signal was tested.

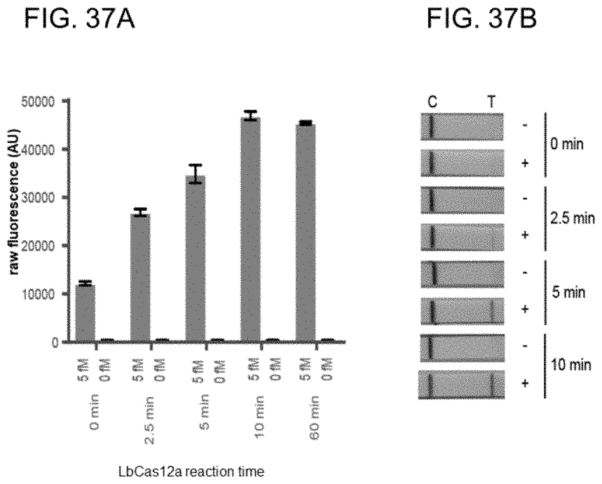

FIG. 33B shows the results of a DETECTR assay using LbCas12a to determine the effect of reaction time for a sample containing either 0 fM SARS-CoV-2 RNA or 5 fM SARS-CoV-2 RNA. Fluorescence signal of LbCas12a detection assay on RT-LAMP amplicon for SARS-CoV-2 N-gene saturated within 10 minutes. RT-LAMP amplicon was generated from 2 μL of 5 fM or 0 fM SARS-CoV-2 N-gene IVT RNA by amplifying at 62° C. for 20 minutes. Visualization of the Cas12 detection reaction was achieved using a FAM-biotin reporter molecule and lateral flow strips designed to capture labeled nucleic acids, as shown in FIG. 33A. Uncleaved reporter molecules are captured at the first detection line (control line), whereas indiscriminate Cas12 cleavage activity generates a signal at the second detection line (test line). To compare the signal generated by Cas12 when using fluorescence or lateral flow, RT-LAMP was performed using 5 fM or 0 fM IVT template using N gene primers and monitored the performance of the Cas12 readout on identical amplicons using a fluorescent plate reader and by lateral flow at 0, 2.5, 5, and 10 minutes. The Cas12 fluorescent signal was detectable in <1 minute, and a visual signal by lateral flow was achieved within 5 minutes. FIG. 37A shows the results of a DETECTR assay using LbCas12a to determine the effect of reaction time for a sample containing either 0 fM SARS-CoV-2 RNA or 5 fM SARS-CoV-2 RNA. Fluorescence signal of LbCas12a (SEQ ID NO: 18) detection assay on RT-LAMP amplicon for SARS-CoV-2 N-gene saturated within 10 minutes. RT-LAMP amplicon was generated from 2 μL of 5 fM or 0 fM SARS-CoV-2 N-gene IVT RNA by amplifying at 62° C. for 20 minutes.

FIG. 33C shows lateral flow test strips assaying samples corresponding to the samples assayed by DETECTR in FIG. 33B. Bands corresponding to control (C) or test (T) are shown for samples containing either 0 fM SARS-CoV-2 RNA (“−”) or 5 fM SARS-CoV-2 RNA (“+”) as a function of reaction time. LbCas12a on the same RT-LAMP amplicon produced visible signal through lateral flow assay within 5 minutes. FIG. 37B shows lateral flow test strips assaying samples corresponding to the samples assayed by DETECTR in FIG. 37A. Bands corresponding to control (C) or test (T) are shown for samples containing either 0 fM SARS-CoV-2 RNA (“−”) or 5 fM SARS-CoV-2 RNA (“+”) as a function of reaction time. LbCas12a (SEQ ID NO: 18) on the same RT-LAMP amplicon as shown in FIG. 37A produced visible signal through lateral flow assay within 5 minutes.

### Example 19

### Detection of SARS-CoV-2 in Patient Samples Using a DETECTR Assay

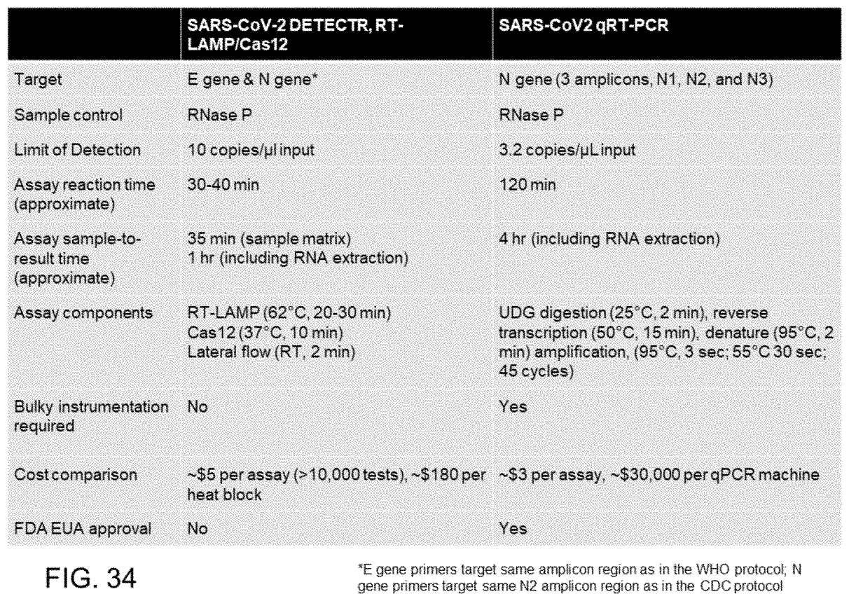

This example describes detection of SARS-CoV-2 in patient samples using a DETECTR assay. Extracted RNA from nasal swab samples collected from six patients with documented SARS-CoV-2 infection, nasal swab samples from 15 patients with other influenza or coronavirus infections, and nasal swab samples from five healthy donors were tested. RNA extracts from patients with influenza (n=4) and other human coronavirus infections (common human seasonal coronavirus infections (OC34, HKU1, 229E, and NL63, n=7)) were compared to in vitro transcribed SARS-CoV-2 target RNA spiked into nasal swab matrix in UTM and RNA extracted from nasal swabs from 2 SARS-CoV-2 infected patients. Samples were detected using SARS-CoV-2 DETECTR assay with fluorescence-based and lateral flow strip readouts FIG. 34 shows a table comparing the SARS-CoV-2 DETECTR assay with RT-LAMP of the present disclosure to the SARS-CoV-2 assay with a quantitative reverse transcription polymerase chain reaction (qRT-PCR) detection method. The N-gene target in the DETECTR RT-LAMP assay is the same as the N-gene N2 amplicon detected in the qRT-PCR assay. FIG. 33E shows patient sample DETECTR data. Clinical samples from 6 patients with COVID-19 infection (n=11, 5 replicates) and 12 patients infected with influenza or one of the 4 seasonal coronaviruses (HCoV-229E, HCoV-HKU1, HCoV-NL63, HCoV-OC43) (n=12) were analyzed using SARS-CoV-2 DETECTR (shaded boxes). Signal intensities from lateral flow strips were quantified using ImageJ and normalized to the highest value within the N gene, E gene or RNase P set, with a positive threshold at five standard deviations above background. Final determination of the SARS-CoV-2 test was based on the interpretation matrix in FIG. 32E. FluA denotes Influenza A, and FluB denotes Influenza B. HCoV denotes human coronavirus. FIG. 33F shows lateral flow test strips testing for SARS-CoV-2 in a patient with COVID-19 (positive for SARS-CoV-2, “patient 11”), a no target control sample lacking the target nucleic acid (“NTC”), and a positive control sample containing the target nucleic acid (“PC”). The E-gene was detected using a gRNA corresponding to SEQ ID NO: 173. The N-gene was detected using a gRNA corresponding to SEQ ID NO: 171. All three samples were tested for the presence of the SARS-CoV-2 N-gene, the SARS-CoV-2 E-gene, and RNase P. There was 100% concordance of the results of the Cas12 based assays with the CDC N2/N3 qRT-PCR assays, demonstrating the feasibility of using the DETECTR Cas12 based assays for diagnosing patients with SARS-CoV-2 infection.

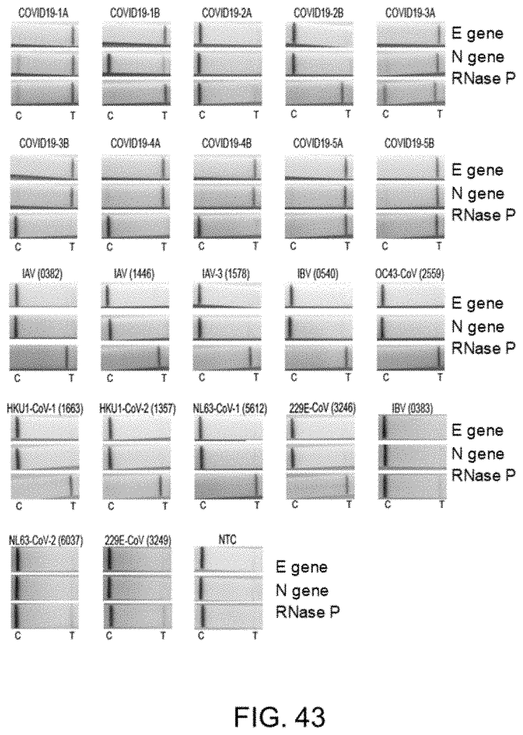

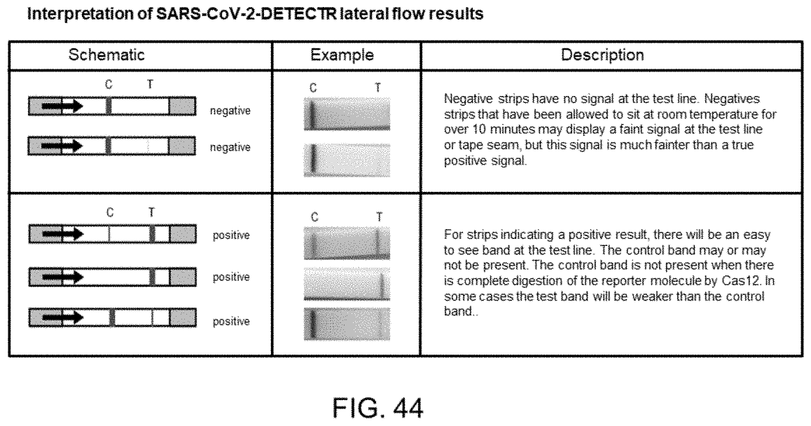

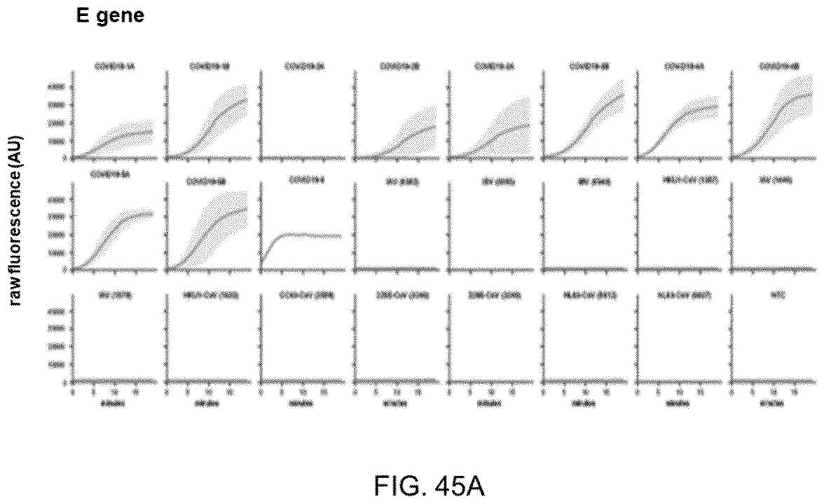

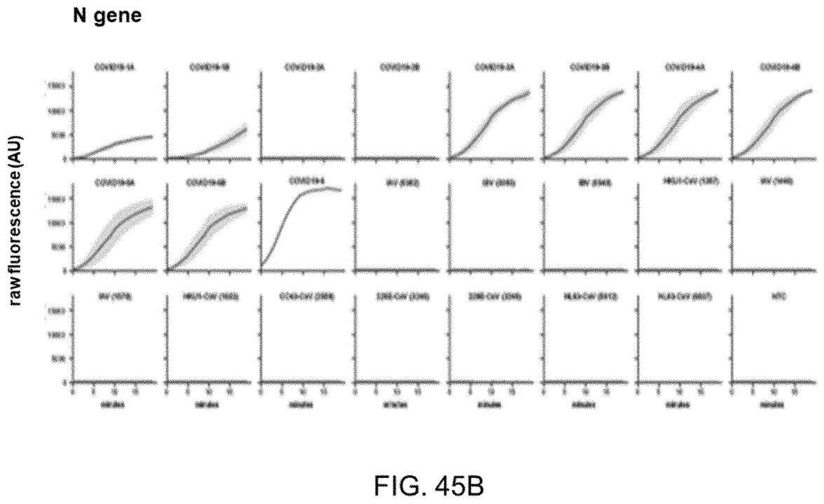

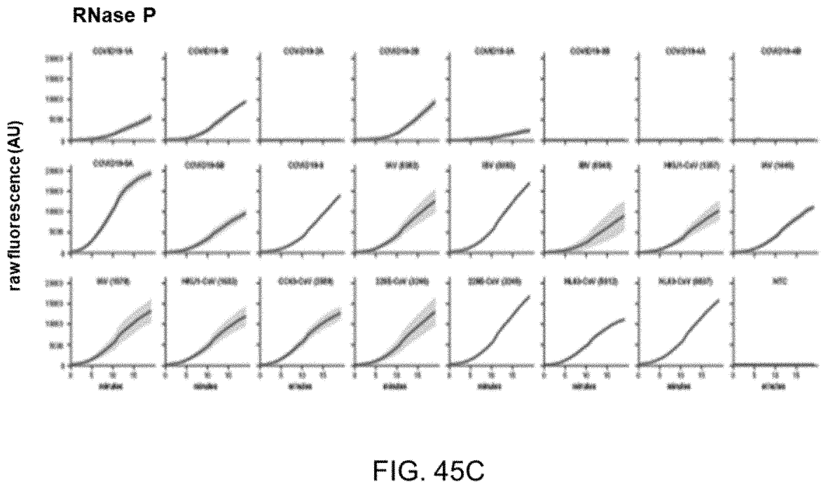

SARS-CoV-2 was detected in 9 of the 11 patient swabs and did not cross react with the other respiratory viruses. The two negative swabs from COVID-19 patients were confirmed to be below the established limit of detection. FIG. 43 shows lateral flow DETECTR results on 10 COVID-19 infected patient samples and 12 patient samples for other viral respiratory infections. Ten samples from 6 patients (COVID19-1 to COVID19-5) with one nasopharyngeal swab (A) and one oropharyngeal swab (B) were tested for SARS-CoV-2 using two different genes, N2 and E as well as a sample input control, RNase P. Results were analyzed in accordance with the guidance provided in FIG. 44. FIG. 44 shows instructions for the interpretation of SARS-CoV-2 DETECTR lateral flow results. FIG. 45A-C show fluorescent DETECTR kinetic curves performed on 11 COVID-19 infected patient samples and 12 patient samples for other viral respiratory infections. Ten nasopharyngeal/oropharyngeal swab samples from 6 patients (COVID19-1 to COVID19-6) were tested for SARS-CoV-2 using two different genes, N2 and E as well as a sample input control, RNase P.

FIG. 45A shows samples tested using the standard amplification and detection conditions, 10 of the 12 COVID-19 positive patient samples resulted in robust fluorescence curves indicating presence of the SARS-CoV-2 E gene (20-minute amplification, signal within 10 min). No E gene signal was detected in the 12 other viral respiratory clinical samples.

FIG. 45B shows samples tested for the presence of the SARS-CoV-2 N gene using an extended amplification time to produce strong fluorescence curves (30-minute amplification, signal within 10 min) for 10 of the 12 COVID-19 positive patient samples. No N gene signal was detected in the 12 other viral respiratory clinical samples.

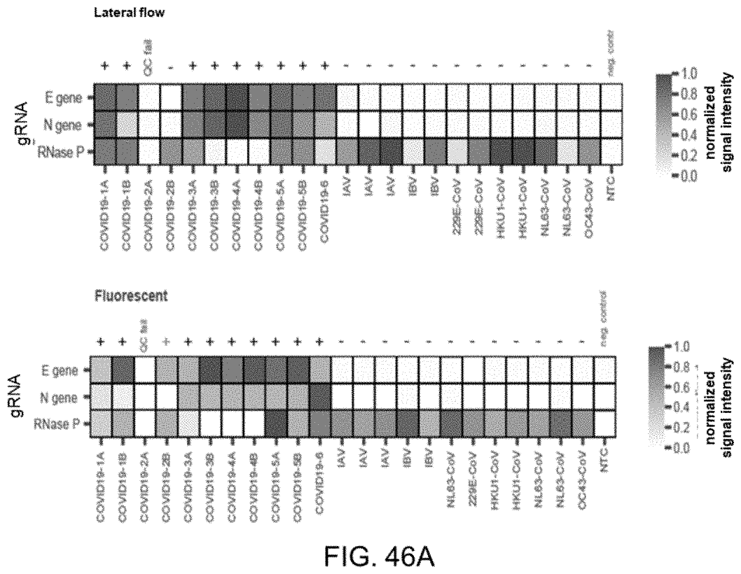

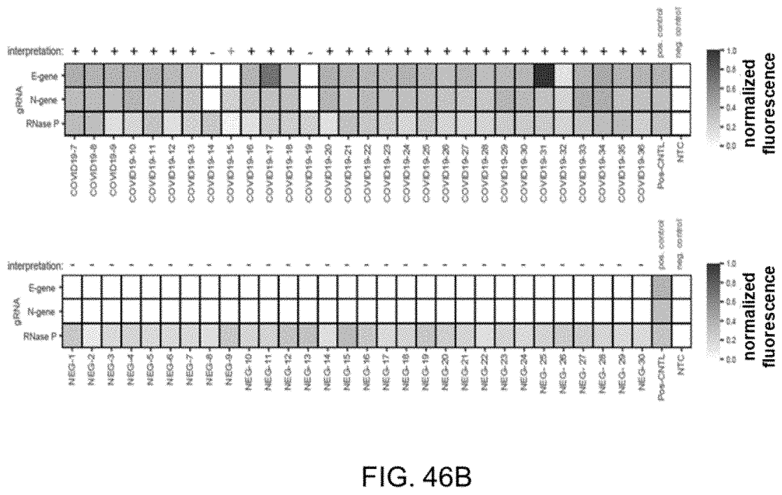

Given the 100% concordance between lateral flow and fluorescence-based readouts shown in FIG. 45 and FIG. 46, a fluorescence-based readout was used to blindly test an additional 60 nasopharyngeal swab samples from patients with acute respiratory infection for SARS-CoV2 using our DETECTR assay. Of the 60 samples, 30 were positive for COVID-19 infection by qRT-PCR testing and 30 were negative for COVID-19 infection but either positive for another viral respiratory infection by respiratory virus panel (RVP) multiplex PCR testing or negative by all testing. The positive predictive agreement (PPA) and negative predictive agreement (NPA) of SARS-CoV-2 DETECTR relative to the CDC qRT-PCR assay were 95% and 100%, respectively, for detection of the coronavirus in 83 total respiratory swab samples.

FIG. 46A shows heatmaps of SARS-CoV-2 DETECTR assay results for clinical samples with the test interpretation indicated. Results of lateral flow SARS-CoV-2 DETECTR assay (top) quantified by ImageJ Gel Analyzer tools for SARS-CoV-2 DETECTR on 24 clinical samples (12 COVID-19 positive) show 98.6% ( 71/72 strips) agreement with the results of the fluorescent version of the assay (bottom). Both assays were run with 30-minute amplification, Cas12 reaction signal taken at 10 min. Presumptive positive indicated by (+) in orange (bottom, column 4).

FIG. 46B shows heatmaps of SARS-CoV-2 DETECTR assay results for clinical samples with the test interpretation indicated. The top plot shows result of fluorescent SARS-CoV-2 DETECTR assay on an additional 30 COVID-19 positive clinical samples (27 positive, 1 presumptive positive, 2 negative). Presumptive positive indicated by (+) in orange (top, column 9). The bottom plot shows result of fluorescent SARS-CoV-2 DETECTR assay on an additional 30 COVID-19 negative clinical samples (0 positive, 30 negative).

Relative to the CDC qRT-PCR protocol, the SARS-CoV-2 DETECTR assay was 90% sensitive and 100% specific for detection of the coronavirus in nasal swab samples, corresponding to positive and negative predictive values of 100% and 91.7%, respectively. FIG. 33G shows performance characteristics of the SARS-CoV-2 DETECTR assay. 83 clinical samples (41 COVID-19 positive, 42 negative) were evaluated using the fluorescent version of the SARS-CoV-2 DETECTR assay. One sample (COVID19-3) was omitted due to failing assay quality control. Positive and negative calls were based on criteria described in FIG. 32E. fM denotes femtomolar; NTC denotes no-template control; PPA denotes positive predictive agreement; NPA denotes negative predictive agreement.

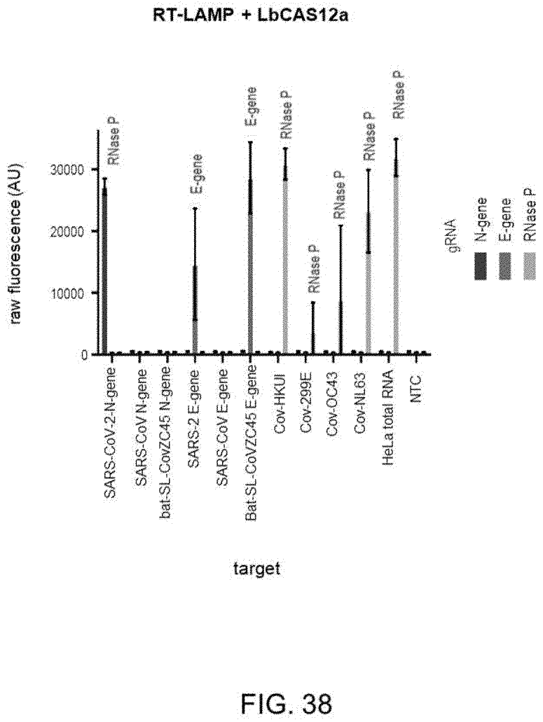

SARS-CoV-2 DETECTR assay (RT-LAMP+Cas12a) was evaluated on IVT RNA products from SARS-CoV-2, SARS-CoV, bast-SL-CoVZC45, and clinical samples from common human coronaviruses. FIG. 38 shows the results of a DETECTR assay to determine the cross-reactivity of gRNAs for different human coronavirus strains. Samples containing in vitro transcribed RNA of the SARS-CoV-2 N-gene, the SARS-CoV N-gene, the bat-SL-CoVZC45 N-gene, the SARS-CoV-2 E-gene, the SARS-CoV E-gene, or the bat-SL-CoVZC45 E-gene, or clinical samples positive for CoV-HKU1, CoV-299E, CoV-OC43, or CoV-NL63 were tested. HeLa total RNA was tested as a positive control for RNase P, and a sample lacking a target nucleic acid (“NTC”) was tested as a negative control. The N-gene was detected using a gRNA corresponding to SEQ ID NO: 171. The E-gene was detected using a gRNA corresponding to SEQ ID NO: 173. RNase P was detected using a gRNA corresponding to SEQ ID NO: 178. The SARS-CoV-2 DETECTR assay was positive only from the in vitro transcribed SARS-CoV-2 spiked samples and nasal swab samples from SARS-CoV-2 infected patients, indicating that the DETECTR assay was specific for SARS-CoV-2. The N-gene was only detected in SARS-CoV-2, whereas the E-gene was detected only in SARS-CoV-2 and bat-SL-CoVZC45. SARS-CoV E-gene was not detected as the RT-LAMP primer set was not capable of amplifying the SARS-CoV E-gene, even though the E-gene gRNA was capable of detecting the SARS-CoV E-gene target site. RNase P was detected in common human coronaviruses because these samples are RNA extracted from clinical samples. Result are shown at 15 minutes of LbCas12a (SEQ ID NO: 18) detection assay signal on fluorescent plate reader.

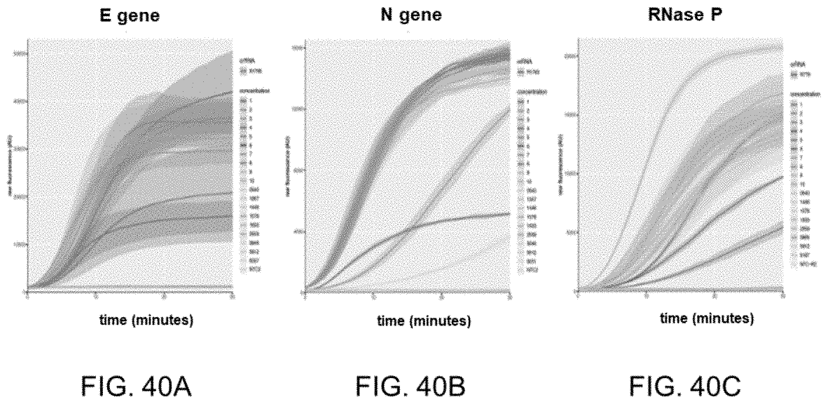

FIG. 40A-FIG. 40B show DETECTR kinetic curves on COVID-19 infected patient samples. Ten nasal swab samples from 5 patients (COVID19-1 to COVID19-10) were tested for SARS-CoV-2 using two different genes, N2 and E as well as a sample input control, RNase P. FIG. 40A shows using the standard amplification and detection conditions, 9 of the 10 patients resulted in robust fluorescence curves indicating presence of the SARS-CoV-2 E-gene (20 minute amplification, signal within 10 minutes). FIG. 40B shows the SARS-CoV-2 N-gene required extended amplification time to produce strong fluorescence curves (30 minute amplification, signal within 10 minutes) for 8 of the 10 patients. FIG. 40C shows that as a sample input control, RNase P was positive for 17 of the 22 total samples tested (20 minute amplification, signal within 10 minutes).

### Example 20

### Improved Detection of an RNase P POP7 Control Gene with Modified LAMP Primers and gRNA

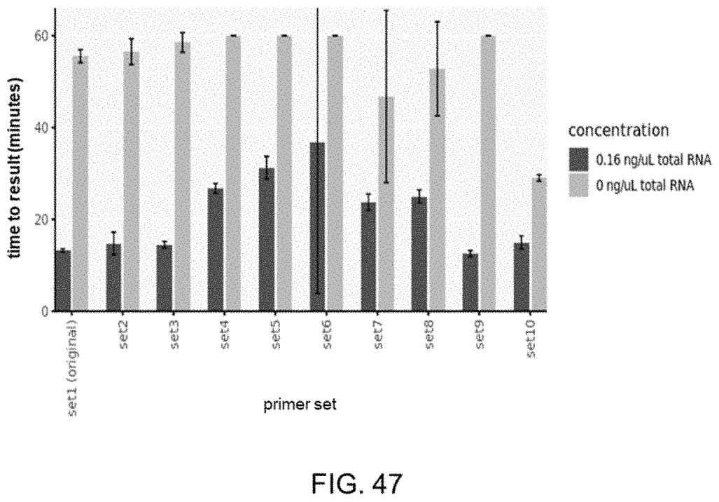

This example describes improved detection of an RNase P POP7 control gene with modified LAMP primers and gRNA. Samples containing RNase P POP7 RNA were assayed using RT-LAMP and DETECTR reactions to assess the amplification and detection efficiency of primer sets and gRNAs directed to RNase P POP7. Samples containing either 0.16 ng/μL total RNA or 0 ng/μL total RNA were amplified by RT-LAMP with different primer sets at 60° C. for 60 minutes. FIG. 47 shows the time to result for RT-LAMP amplification of RNase P POP7 with different primer sets. Time to result was determined for samples amplified with primer sets 1-10. Primer set 1 corresponds to SEQ ID NO: 206-SEQ ID NO: 211, and primer set 9 corresponds to SEQ ID NO: 212-SEQ ID NO: 217. Primer set 9 showed improved time to result over primer set 1 and primer sets 2-8 and 10 for samples containing 0.16 ng/μL total RNA. Additionally, primer set 9 showed less non-specific amplification of samples without total RNA (0 ng/μL total RNA) than primer set 1 and primer sets 2, 3, 7, 8, and 10.

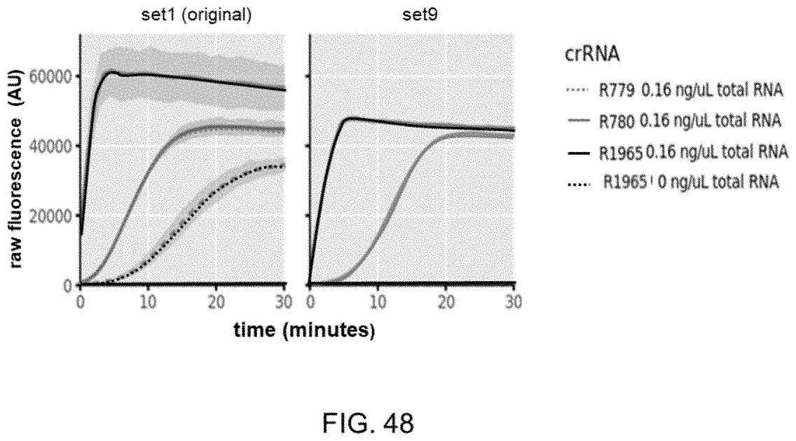

A DETECTR reaction was performed on the amplicons generated by RT-LAMP. Samples were detected using gRNAs corresponding to R779 (SEQ ID NO: 178), R780 (SEQ ID NO: 219.), or R1965 (SEQ ID NO: 218). FIG. 48 shows raw fluorescence over time of a DETECTR reaction performed on RNase P POP7 amplified using RT-LAMP with primer set 1 or primer set 9 and detected with R779, R780, or R1965 gRNAs. The DETECTR reaction was carried out at 37° C. for 90 minutes. The amplicon generated by the set 1 primers were detected without background (dotted line) by R779. Clean detection was also seen by R1965 and R780 on amplicons generated by set 9. The results show that R1965 detects faster than R779 or R780.

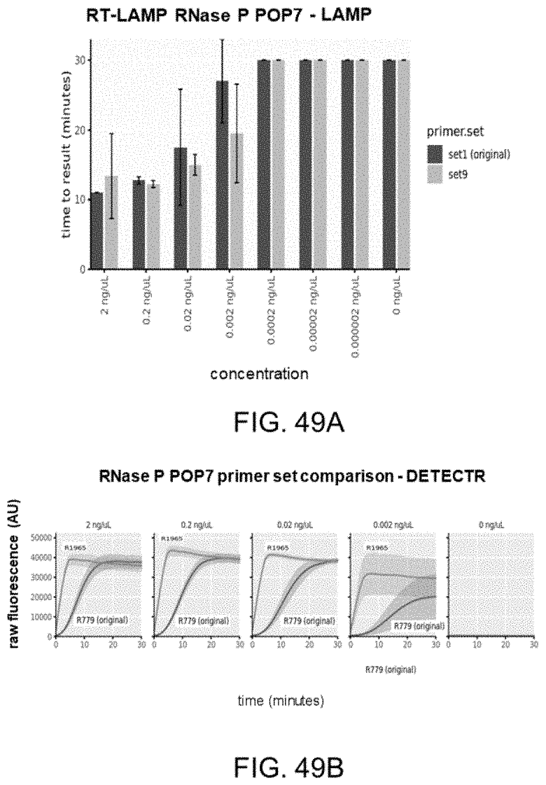

The limit of detection was then tested for RNase P POP7 amplified using RT-LAMP with primer set 1 (SEQ ID NO: 206-SEQ ID NO: 211) or primer set 9 (SEQ ID NO: 212-SEQ ID NO: 217) and detected with R779 gRNA (SEQ ID NO: 178) or R1965 gRNA (SEQ ID NO: 218). FIG. 49A shows the time to result of RNase P POP7 detection in samples containing 10-fold dilutions of total RNA amplified using RT-LAMP with primer set 1 or primer set 9. Amplification was carried out at 60° C. for 30 minutes. FIG. 49B shows a DETECTR reaction of the RNase P POP7 amplicons shown in FIG. 49A and detected using gRNA 779 (SEQ ID NO: 178) or gRNA 1965 (SEQ ID NO: 218). Samples amplified using primer set 1 were detected with gRNA 779 and samples amplified with primer set 9 were detected with gRNA 1965. The DETECTR reaction was carried out at 37° C. for 90 minutes. Primer set 9 showed improved time to limit of detection, as seen by a faster time to result at low RNA concentrations, compared to primer set 1. Additionally, primer set 9 showed improved speed and sensitivity in a DETECTR reaction when detected with gRNA 1965 as compared to samples amplified with primer set 1 and detected with gRNA 779.

### Example 21

### Viral Lysis Buffer for Lysis and Amplification of a Coronavirus

This example describes a viral lysis buffer for lysis and amplification of a coronavirus. Nasal swab or saliva samples are collected from individuals suspected of having a coronavirus infection. Nasal swab and saliva samples are suspended in a viral lysis buffer formulated to lyse the viral capsids and release the viral genome. The viral lysis buffer is compatible with RT-LAMP amplification of the viral genome and DETECTR detection of a target nucleic acid, providing a one-step sample preparation solution for a coronavirus DETECTR reaction.

### Example 22

### Amplification of a Target Nucleic Acid in a Viral Lysis Buffer

This example describes amplification of a target nucleic acid in a viral lysis buffer. The effects of various buffer compositions, reducing agents, and incubation temperatures were tested on amplification of a target nucleic acid. Samples in different buffers were amplified using LAMP amplification, and the resulting fluorescence was measured. Higher fluorescence was indicative of more amplification.

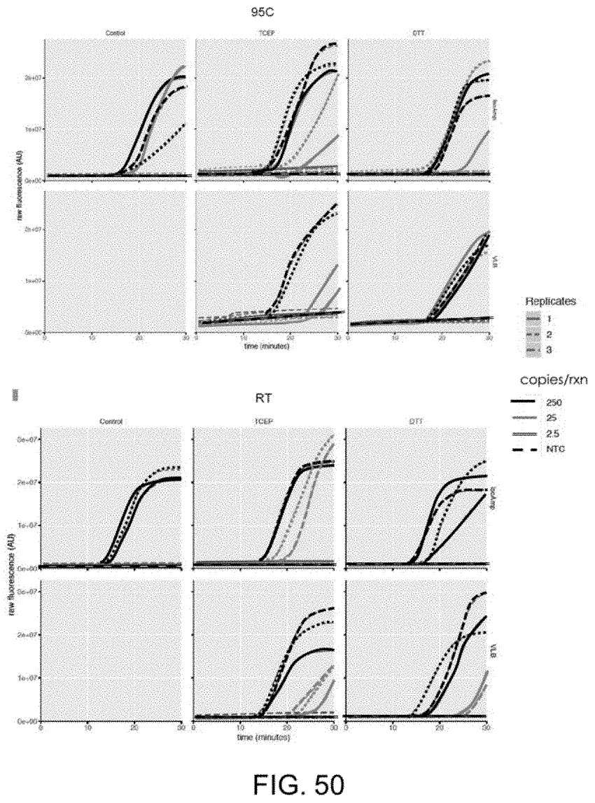

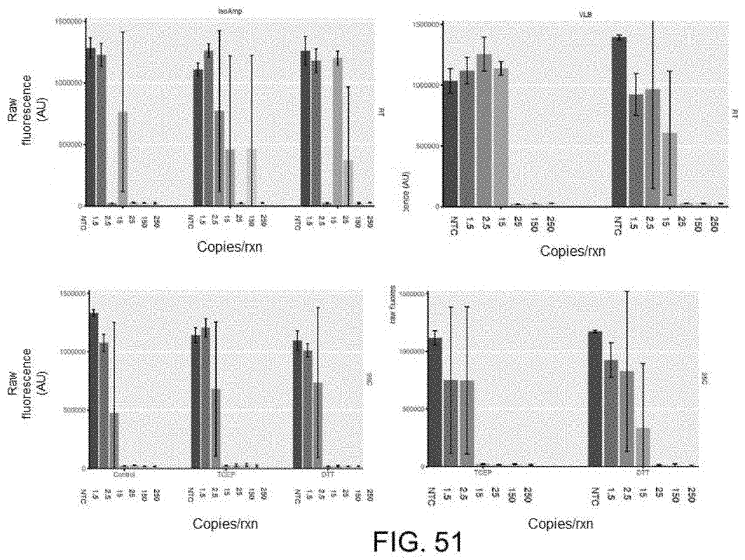

FIG. 50 shows the results of amplification of a SeraCare target nucleic acid using LAMP under different lysis conditions. Samples were amplified in various buffers. Samples were incubated for 5 minutes at either room temperature (left plots) or 95° C. (right plots). Samples containing either no target (“NTC”), 2.5, 25, or 250 copies per reaction. Assays were performed in triplicate using 5 μL of sample in a 25 μL reaction. FIG. 51 shows the results of amplification of a SeraCare standard target nucleic acid using LAMP under different lysis conditions. Samples were amplified in various buffers. Samples were incubated for 5 minutes at either room temperature (top plots) or 95° C. (bottom plots). Samples containing either no target (“NTC”), 1.5, 2.5, 15, 25, 150, or 250 copies per reaction. Assays were performed in triplicate using 3 μL of sample in a 15 μL reaction or 5 μL of sample in a 25 μL reaction.

The results of this experiment demonstrated that certain buffers were more conducive to LAMP amplification.

### Example 23

### Amplification of a Target Nucleic Acid from COVID-19 Patient Samples in a Viral Lysis Buffer

This example describes amplification of a target nucleic acid from COVID-19 patient samples in a viral lysis buffer. Samples collected from patients positive for COVID-19 were lysed and amplified in viral lysis buffers with varying components. Target nucleic acids corresponding to the SARS-CoV-2 N gene and RNaseP were amplified using LAMP as described in EXAMPLE 22. Various buffer formulations were tested.

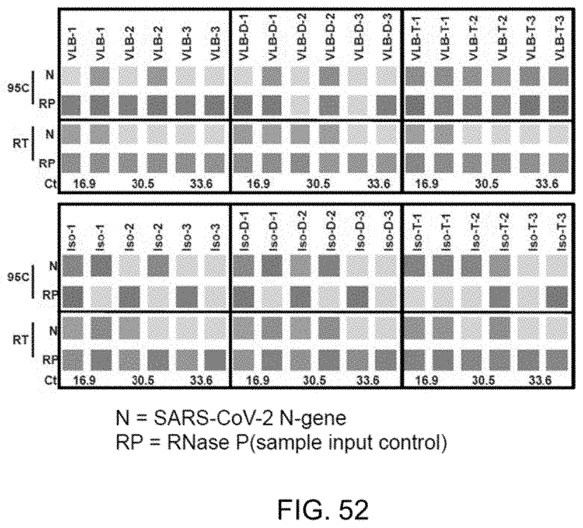

FIG. 52 shows amplification of a SARS-CoV-2 N gene (“N”) and an RNase P sample input control nucleic acid (“RP”) in the presence of six different viral lysis buffers (“VLB,” “VLB-D,” “VLB-T,” “Buffer,” “Buffer-A,” and “Buffer-B”). Buffer-A contains Buffer with Reducing Agent A and Buffer-B contains Buffer with Reducing Agent B. Shaded squares indicate rate of amplification, with darker shading indicating faster amplification. Amplification was performed at either 95° C. (“95 C”) or room temperature (“RT”) on high, medium, or low titer COVID-19 positive patient samples (“16.9,” “30.5,” and “33.6,” respectively). Samples were measured in duplicate. Of the buffers tested, fastest amplification was observed in VLB-T at 95° C.

### Example 24

### Detection of a SNP Using a DETECTR Assay on a Microfluidic Cartridge

This example describes detection of a SNP using a DETECTR assay on a microfluidic cartridge. This assay was performed on a microfluidic cartridge shown in FIG. 53B. A cartridge manifold for configured to heat the cartridge was turned on. 5 μL of a sample from a blue-eyed individual was combined with 45 μL of a LAMP master mix solution containing the components for LAMP amplification of the sample. The sample was pre-mixed before being added to the cartridge. The pre-mixed sample was loaded into the cartridge in the amplification chamber, and the chamber was sealed with clear tape. 95 μL of blue eye RNP (G SNP) was loaded into the DETECTR chamber. The loaded chip was transferred onto the pre-heated manifold and sealed with clear tape.

The first heater of the manifold was set to 60° C., and the second heater was set to 37° C. The sample was incubated for 30 minutes at 60° C. After 30 minutes, a first pump in the manifold was initiated to pump the LAMP buffer with the sample through the cartridge. A second pump in the manifold was initiated to push 95 μL of the DETECTR solution into the detection chamber. The sample was incubated at 37° C. for 30 minutes. Fluorescence was visualized using a black box fluorescence detector.

A control assay was performed in microcentrifuge tubes using a heating block. In a first tube, 5 μL of a sample from a blue-eyed individual was combined with 45 μL of a LAMP master mix solution. In a second tube, 5 μL of a sample from a brown-eyed individual was combined with 45 μL of a LAMP master mix solution. Samples were incubated for 30 minutes at 60° C. in a mini dry bath. 5 μL of each amplified sample was transferred to 95 μL of a 1×RNP solution for detection of A and G SNPs. The reactions were transferred to a 37° C. heat block.

### Example 25

### Amplification and Detection of a SNP in a Microfluidic Cartridge

This example describes amplification and detection of a SNP in a microfluidic cartridge. These assays were performed in the microfluidic cartridge illustrated in FIG. 55B. The following solutions were prepared: LAMP master mix (1× IsoAmp Buffer (NEB), 4.5 mM MgSO₄, 1.4 mM dNTPs, 1:5 Bst 2.0 (NEB), 1× primer master mix, and 1:10 target DNA), and CRISPR complex (1× MBuffer3, 40 nM crRNA, and 40 nM Cas12 variant (SEQ ID NO: 28); 1 μM reporter substrate was added after incubated at 37° C.).

PMMA layers of the cartridge were cleaned by immersion in RNAse Zap for 20 minutes and washing of remnants of the cleaning solution by washing twice in nuclease free water. The cartridge was dried using a stream of nitrogen. The layers of the cartridge were assembled. The top half of the CRISPR reaction workflow was blocked with high sol epoxy and dried for 20 minutes until clear. 80 μL of LAMP master mix was pre-mixed in a microcentrifuge tube with 10 μL of primer mix and 10 μL of pure DNA extract. The solution was mixed by pipetting up and down. 70 μL of this solution was loaded into the amplification chamber of the cartridge using a pipette. The chamber was sealed using a small rectangular piece of PCR adhesive (Biorad, MSB-1001).

The cartridge was placed into a heating manifold, and the aluminum block was heated to an on-chip temperature of 60° C. The sample was incubated at 60° C. for 30 minutes to amplify the sample using LAMP. 100 μL of CRISPR reagent containing a blue-eye gRNA was added to the lower DETECTR chamber. The top and bottom chambers were sealed with small rectangular pieces of PCR adhesive. The CRISPR reagents were mixed with 5 μL of the amplified sample by actuating a valve in the cartridge. The manifold was covered with a shroud of 3D printed APS to block light. The aluminum block was heated to an on-chip temperature of 37° C. The CRISPR reaction was incubated for 30 minutes at 37° C. The resulting fluorescence was observed by eye.

The assay was repeated as described above using the cartridge illustrated in FIG. 55C, except that the top half was not sealed with epoxy. In both assays, the fluorescence corresponding to a positive result was observable by eye. Illumination of the cartridges in the manifold from the top of the cartridge resulted in uneven illumination of the detection chambers.

### Example 26

### Amplification and Detection of a SNP in a Revised Microfluidic Cartridge

This example describes amplification and detection of a SNP in a revised microfluidic cartridge. This assay was performed on a microfluidic cartridge illustrated in FIG. 56A. LAMP master mix and CRISPR complex solutions were prepared as described in EXAMPLE 25. PMMA layers of the cartridge were cleaned by immersion in RNAse Zap for 20 minutes and washing of remnants of the cleaning solution by washing twice in nuclease free water. The cartridge was dried using a stream of nitrogen. The layers of the cartridge were assembled.

40 μL of LAMP master mix was pre-mixed in a microcentrifuge tube with 5 μL of primer mix and 5 μL of pure DNA extract. The solution was mixed by pipetting up and down. 50 μL of this solution was loaded into the amplification chamber of the cartridge using a pipette. The chamber was sealed using a small rectangular piece of PCR adhesive (Biorad, MSB-1001). 95 μL of the CRISPR reagent solution containing a Cas12 variant (SEQ ID NO: 28) and a gRNA directed to a brown-eye SNP was added to the lower DETECTR chamber, and 95 μL of a negative reagent solution (5× MBuffer3) was added to the upper DETECTR chamber. The chamber was sealed using a small rectangular piece of PCR adhesive (Biorad, MSB-1001).

The cartridge was assembled on a heating manifold, and the aluminum block was heated to an on-chip temperature of 60° C. at the amplification chamber. Heating was initiated 2 minutes prior to beginning the assay. Amplification was performed at 60° C. for 30 minutes. The valve of the cartridge was actuated to mix the CRISPR reagent with 5 μL of the amplified sample. The manifold heater of the detection chamber was heated to 37° C. without pre-heating. The DETECTR reaction was performed at 37° C. for 30 minutes, and the resulting fluorescence was observed by eye. The chambers were imaged by illuminating with either an LED from a mini PCR kit or an LED from ThorLabs.

The assay was repeated on a new cartridge of the same design with the following modifications: the CRISPR reagents were not preloaded into the device, because the heater was still warm from the previous run, and the amplification and detection steps were run for 15 minutes instead of 30 minutes.

A third assay was performed on a microfluidic cartridge illustrated in FIG. 56B. The amplification chamber was loaded with 50 μL of nuclease free water and the chamber was sealed with a small piece of PCR adhesive. 50 μL of 1 μM ATTO-488 dye and 45 μL of nuclease free water were loaded into the lower CRISPR chamber, and 95 μL of nuclease free water was loaded into the upper CRISPR chamber. Both chambers were sealed with a small piece of PCR adhesive. The cartridge was assembled on a heating manifold, as shown in FIG. 64B. Samples were incubated for 10 seconds in the amplification chamber. The first pump was run for 3 seconds to drive 5 μL of fluid out of the amplification chamber and into the CRISPR chamber (also referred to as the detection chamber). The second pump was run for 5 seconds to drive detection reagents into the CRISPER chamber. The samples were incubated in the CRISPR chamber for 10 seconds before illuminating with an LED. The assay was repeated with the following parameters: 30-minute incubation in the amplification chamber, pump 1 run for 1 second, pump 2 run for 20 seconds, and 15-minute incubation in the CRISPR chamber before illuminating with an LED. The longer pump times improved fluid transfer between chambers.

### Example 27

### Use of a Microfluidic Device for a DETECTR Reaction

This example describes use of a microfluidic device for a DETECTR reaction. A microfluidic cartridge as illustrated in any of FIG. 53A, FIG. 53B, FIG. 54A, FIG. 54B, FIG. 55A, FIG. 55B, FIG. 55C, FIG. 55D, FIG. 56A, FIG. 56B, FIG. 56C, or FIG. 56D is loaded with amplification reagents and DETECTR reagents. 50 μL of amplification reagent is added to the amplification chamber, and 95 μL of DETECTR reagent is added to DETECTR chamber. The wells of the cartridge are sealed. The cartridge is loaded into a heating manifold as illustrated in any of FIG. 63A, FIG. 63B, FIG. 64B, or FIG. 65. The cartridge is inserted in a specific orientation. Screws are tightened to hold the cartridge in place. Openings are sealed with clear qPCR take cut to size to create an air-tight seal. A thermocouple is inserted into the amplification chamber to record temperatures. The solenoid, shown in FIG. 57A, is energized to close the valve. Indicator LED lights turn on. Two heaters, set to 60° C. and 37° C., are turned on. The sample is incubated at 60° C. for 30 minutes in the amplification chamber. The solenoid is de-energized to open the valve. Pump 1 is activated for 15 seconds to move fluid from the amplification chamber to the DETECTR reaction chambers. After 15 seconds, pump 2 is activated for 15 seconds to move fluid from the DETECTR reagent reservoirs to the DETECTR reaction chambers. The sample is incubated in the DETECTR reaction chambers for 30 minutes at 37° C. The indicator light turns off. The LED is turned on and fluorescence is measured by image, visual assessment, or photodiode detection.

At the end of the 30-minute 60° C. LAMP incubation, the solenoid valve opens and the peristaltic pump #1 engages at 100% PWM for 10 seconds. The LAMP buffer is pumped through the valve to the intersection of the serpentine channels leading to the DETECTR reaction chambers and the straight channels leading to the DETECTR reagent reservoirs. The serpentine channel leading to the DETECTR reaction chambers has a larger cross-sectional area than the channel leading to the DETECTR reagent reservoirs. This is intended to reduce the fluidic resistance in the serpentine channels and direct all of the buffer towards the DETECTR reaction chambers. However, throughout this study (testing 23+ chips), the buffer has split both ways nearly every time, with approximately half the buffer volume going the wrong way. In the next fluidic step, the solenoid valve closes and DETECTR reagent is pumped towards the DETECTR reaction chambers, collecting the LAMP product along the way. This provides some mixing as both buffers travel through the serpentine channels simultaneously, but this process also creates bubbles that can get carried to the DETECTR chamber.

To prevent bubbles from interfering with fluorescence measurements during DETECTR, a larger volume of buffer is loaded into the reservoirs than the reaction chambers can fit and use a longer pumping time than necessary. This ensures that the chambers are completely filled with reagent and all bubbles have been popped. The DETECTR reaction chambers have a 70 μL volume, and 25 μL LAMP plus 95 μL DETECTR reagent are delivered into each chamber. The second fluidic step (DETECTR reagent to the DETECTR reaction chambers) takes about 20-30 seconds to deliver all the buffer, but this step is run for 45 seconds. This results in completely full DETECTR reaction chambers, with the excess reagents backed up in the serpentine channels. In addition to bubbles, if the DETECTR reaction chambers are not completely filled, condensation forms on the top of the chamber during the 37° C. incubation, which also interferes with fluorescence measurements taken from above.

### Example 28

### Thermal Testing of a Microfluidic Device for a DETECTR Reaction

This example describes thermal testing of a microfluidic device for a DETECTR reaction. The thermal performance of a heating manifold was tested by measuring the time to temperature and the accuracy of heating to the setpoints with thermocouples submerged within the buffer. Under standard assay temperature setpoints (60° C. LAMP/37° C. DETECTR), the LAMP buffer heats to 60° C. in 8.5 minutes, but the DETECTR buffer reaches a maximum temperature of 34° C. at around 21 minutes. This is somewhat counterintuitive, since it takes longer to hit a lower temperature (and the DETECTR buffer does not reach the setpoint temperature). To hit a specific temperature, the heater controller varies the amount of time it spends in the on state. This state switching is quantified by the pulsed width modulation (PWM) value, the percentage of a given unit of time it spends in the on state. The heater controller also samples the temperature of the heater for feedback on the difference between the current temperature and the setpoint temperature. The larger the difference between those two values, the higher the resulting PWM value will be. As the heater temperature approaches the setpoint, the PWM value drops to slow the rate of change and avoid overshooting the setpoint temperature. The difference between the room temperature heater and the LAMP setpoint is about 35° C., while the difference between the DETECTR heater and its setpoint is about 12° C. The LAMP incubation heats with maximum PWM values around 20%, and the DETECTR incubation heats with maximum PWM values around 12%. Our current setup is designed with a larger emphasis on accuracy and not overshooting the setpoint temperature than heating the buffer to assay temperature quickly.

Specific PWM values can be used to heat to our setpoint temperatures faster. However, this is a manual process and can result in overshooting the target temperatures and damaging the breadboard prototype and melting the microfluidic chip. With the LAMP heater PWM value set to 100%, the LAMP buffer (measured by thermocouple) heats to 60° C. in 90 seconds, but the heater temperature hits 100° C. With the DETECTR heater PWM set to 100%, the DETECTR buffer heats to 37° C. in 60 seconds, and the heater hits 80° C. Turning the heater off when the DETECTR buffer hits 37° C. results in a maximum buffer temperature of around 60° C. the temperature of the DETECTR side of the chip rises during the 30-minute 60° C. LAMP incubation so that it is higher than room temperature. It varies from time to time, but it is usually between 25-29° C. by the beginning of the DETECTR side.

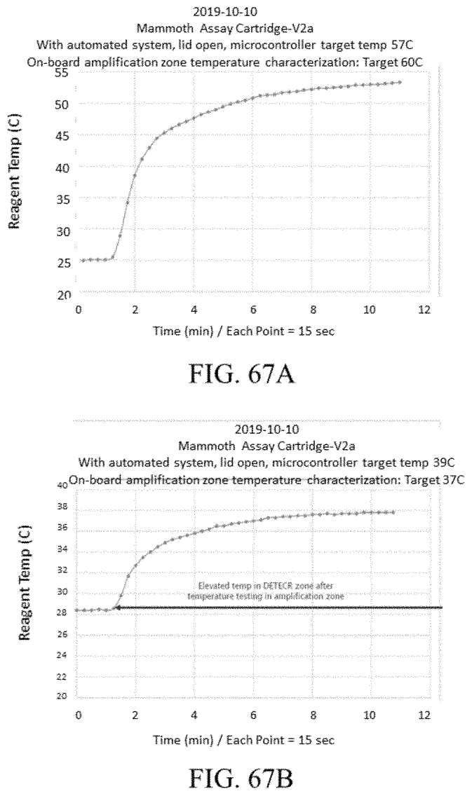

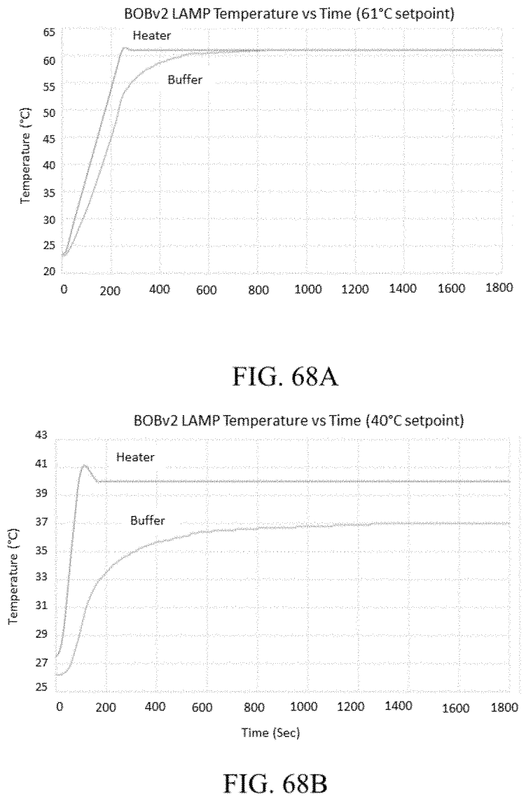

FIG. 67A, FIG. 67B, FIG. 68A, and FIG. 68B show thermal testing summaries for an amplification chamber heated to 60° C. (FIG. 67A and FIG. 68A) or a DETECTR chamber heated to 37° C. (FIG. 67B and FIG. 68B). FIG. 68A shows a graph titled BOBv2 LAMP Temperature vs Time (61° C. setpoint). The x-axis shows time in seconds from 0 to 1800 in increments of 200. The y-axis shows temperature in ° C. ranging from 20 to 65 in increments of 5. The graph includes two lines representing heater and buffer. While both the heater and buffer lines reach the same temperature eventually, the heater line achieves the max temperature more quickly. FIG. 68B shows a graph titled BOBv2 LAMP Temperature vs Time (40° C. setpoint). The x-axis shows time in seconds from 0 to 1800 in increments of 200. The y-axis shows temperature in ° C. ranging from 25 to 43 in increments of 2. The graph includes two lines representing heater and buffer. The heater line reaches a higher temperature more quickly.

### Example 29

### Detection of a HERC2 SNP Using a Microfluidic Cartridge

This example describes detection of a HERC2 SNP using a microfluidic cartridge. A primer mix containing 2 μM F3 primer, 2 μM B3 primer, 16 μM FIP primer, 16 μM BIP primer, 8 μM LF primer, and 8 μM LB primer in nuclease free water was prepared. A complexing reaction containing 1×MBuffer3, 40 nM crRNA, and 50 nM Cas12 variant (SEQ ID NO: 28) was prepared. 40 nM reporter substrate was added after incubating at 37° C. for 30 minutes. A LAMP mix containing 1×IsoAmp Buffer, 4.5 mM MgSO₄, dNTPs, and 1×primer mix was prepared. DETECTR reagents were loaded into a microfluidic cartridge and wells were sealed with PCR tape. LAMP mix was mixed with primers and loaded into the cartridge. The narrow end of the Chip Shop tank was covered with parafilm and inserted into the luer connection above the LAMP reaction chamber. The Chip Shop tank was loaded with 200 μL of 20 mM NaOH. The cartridge was inserted into the heating manifold and screws were tightened. A buccal swab was added to the tank, gently agitated, and incubated for 2 minutes. A Drummond micropipette was used to deliver 10 μL of lysed sample through parafilm into LAMP reaction chamber. The tank was removed and the chamber was sealed with qPCR tape cut to size.

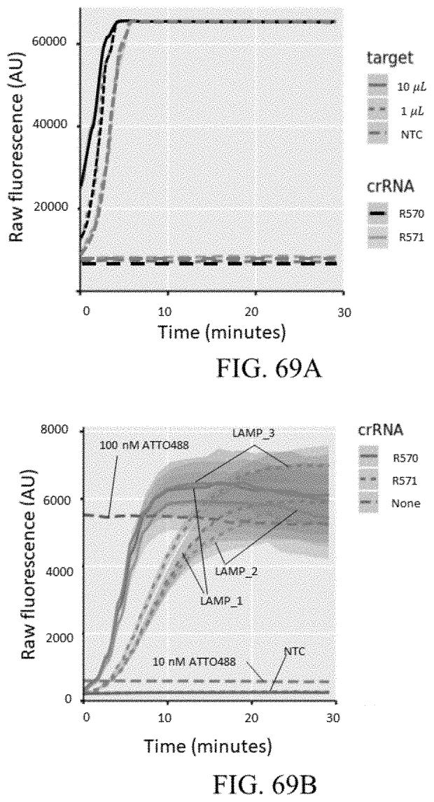

FIG. 69A shows the DETECTR results run on a plate reader at a gain of 100, using the LAMP product from the microfluidic cartridge as an input. The samples were run in duplicate with a single non-template control (NTC). 19 μL of the DETECTR master mix (the same mixture used on the device) was pipetted into wells of a 384-well plate and 1 μL of LAMP amplicon was added. For one sample, 10 μL of amplicon was inadvertently added; that sample is represented by “10 μL target”. Because the donor is homozygous for the A-SNP, guide R570 was expected to generate a faster signal than R571. A slight difference was observed between the two samples. FIG. 69A shows a line graph with the x-axis showing time in minutes ranging from 0 to 30 in increments of 10 and the y-axis shows raw fluorescence in arbitrary units (AU) ranging from 0 to 60000 in increments of 20000. The bottom two flat lines are R570 NTC and R571 NTC. The lines achieving high signal rapidly include, from left to right, R570 10 ul, R 570 1 ul, and R571 1 ul.

FIG. 69B shows three LAMP products run on a plate reader using samples from a microfluidic chip. The LAMP reactions are numbered in the order that the chips were run (LAMP_1 was run first, etc.). The donor was homozygous for SNP A and, accordingly, crRNA 570 comes up first. The ATTO 488 was used as a fluorescence standard. These measurements were taken on a plate reader at a gain of 60. Results of the three LAMP reactions were clustered close together, which indicated good run-to-run reproducibility for amplification on the microfluidic cartridge and heating manifold. Each LAMP reaction was run in triplicate with each crRNA, generating the error ranges visible in the graph. FIG. 69B shows a line graph with the x-axis showing time in minutes ranging from 0 to 30 in increments of 10 and the y-axis shows raw fluorescence in arbitrary units (AU) ranging from 0 to 8000 in increments of 2000. The flat lines near the bottom of the graph are 10 nM ATTO488 None and NTC. The flat dashed line near 6000 AU is 100 nM ATTO488 None. The lines achieving high signal rapidly include, from left to right approximately, LAMP_1 R570 and LAMP_3 R570, LAMP_2 R570, LAMP_3 R571, LAMP_1 R571, and LAMP_2 R571.

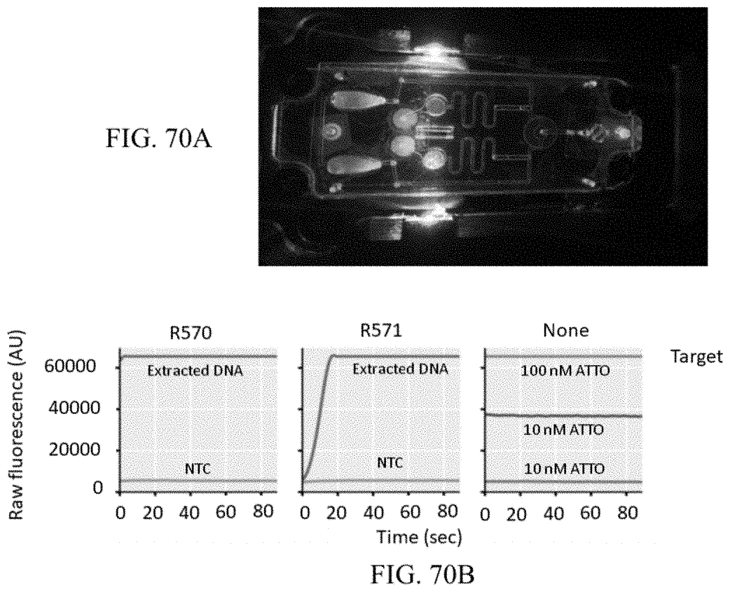

Another assay was performed. Solutions were prepared as described above, and samples were run on a microfluidic cartridge shown in FIG. 56A with addition of a luer connector on top of the amplification chamber. A buccal swab sample was prepared as described above. The cartridge was loaded, and the assay was run with the following settings: 30 minutes amplification, 10 seconds Pump 1, 40 seconds Pump 2, 30 minutes DETECTR. Samples were measured on a plate reader. FIG. 70A an image of the microfluidic cartridge after the assay. The bluer appearance of the right well compared to the green appearance of the left well is likely due to the bubbles in the right well diffusing the input blue light. FIG. 70B shows results of a DETECTR reaction measured on a plate reader after 30 minutes of LAMP amplification. The bubbles in the one reaction chamber interfered with the signal from the ESE log, so the quantitative measurements shouldn't be trusted. However, the 10 minute and 20-minute timepoints had similar signals. Furthermore, both wells appeared visually bright when the LEDs turned on after 30 minutes of DETECTR. The DETECTR results on the plate reader showed that after 30 minutes the signal was high for both SNPs. FIG. 70B shows line graphs from left to right titled R570, R571, and None. The x-axis on each graph shows time in seconds ranging from 0 to 80 in increments of 20 and the y-axis on each graph shows raw fluorescence in arbitrary units (AU) ranging from 0 to 60000 in increments of 20000. In the leftmost graph, the NTC line is flat at the bottom, while the extracted DNA line achieves high fluorescence signal rapidly. In the middle graph, the NTC line is flat at the bottom, while the extracted DNA line achieves high fluorescence signal rapidly. In the right graph, the 10 nM ATTO line is flat at the bottom, the 10 nM ATTO line is flat near the middle, and the 100 nM ATTO line is flat at the top.

### Example 30

### Detection of a Coronavirus Using a Microfluidic Cartridge

This example describes detection of a coronavirus using a microfluidic cartridge. A complexing reaction containing 1× MBuffer3, 40 nM crRNA, and 50 nM Cas12 variant (SEQ ID NO: 28) was prepared. 40 nM reporter substrate was added after incubating at 37° C. for 30 minutes. 95 μL DETECTR reagents were loaded into each DETECTR reagent well and sealed with qPCR tape. A tube of N Gene LAMP master mix (537 μL) was mixed with 32 μL of 100 mM MgSO₄ and 40 μL of mixture was loaded into a cartridge. 10 μL of Twist SARS-Cov-2 standard was added at various copies/μL or 1×TE as a negative control to LAMP reaction chamber. The cartridge was inserted into the manifold and tightened. The LAMP reaction chamber was sealed with qPCR tape. Temperatures were set (62° C. LAMP, 40° C. DETECTR (to account for thermal offset)) and automated workflow was initiated. A 3D-printed optical cover was placed on the cartridge to minimize optical noise. DETECTR measurements were taken at 0 min, 2 min, 5 min, 10 min, 20 min, and 30 min. The copy number of RNA in the LAMP reaction was varied in order to estimate the lower limit of detection in the device.

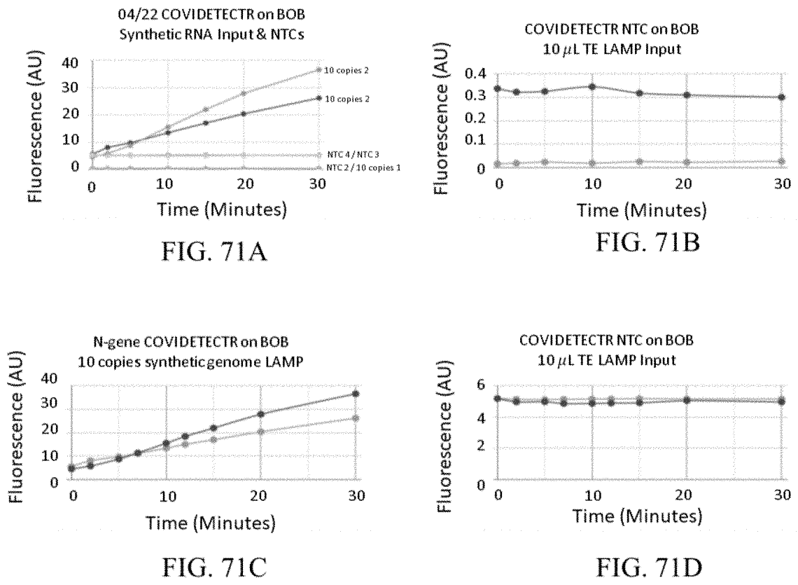

FIG. 71A, FIG. 71B, FIG. 71C, and FIG. 71D show results of the coronavirus DETECTR reaction. The two reaction chambers with 10 copies input to LAMP resulted in a rapidly increasing DETECTR signal. All NTCs were negative. With 10 copies input into LAMP, the DETECTR signal gradually increased over the course of the reaction, as shown in the photodiode measurements below in FIG. 71C. The negative controls in FIG. 71D indicated an absence of contamination.

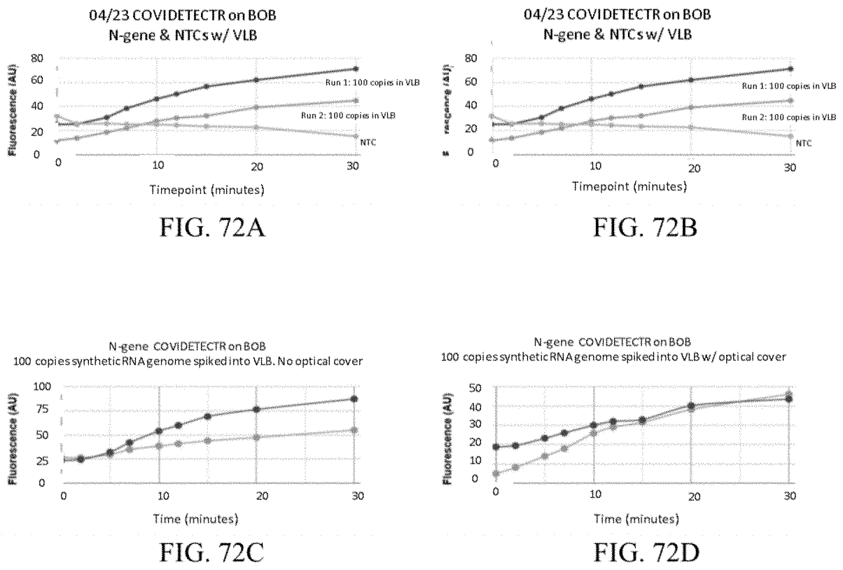

The assay was repeated. FIG. 72A, FIG. 72B, FIG. 72C, and FIG. 72D show the results of the repeated coronavirus DETECTR reaction.

### Example 31

### Turnaround Time of an Influenza B DETECTR Assy in a Microfluidic Cartridge

This example describes the turnaround time of an influenza B DETECTR assay in a microfluidic cartridge. A primer mix containing 2 μM F3 primer, 2 μM B3 primer, 16 μM FIP primer, 16 μM BIP primer, 8 μM LF primer, and 8 μM LB primer in nuclease free water was prepared. A complexing reaction containing 1×MBuffer3, 40 nM crRNA, and 50 nM Cas12 variant (SEQ ID NO: 28) was prepared. 40 nM reporter substrate was added after incubating at 37° C. for 30 minutes. 95 μL DETECTR reagents were loaded into each DETECTR reagent well and sealed with qPCR tape. 40 μL of LAMP mixture was added to the cartridge. 2 μL of 1 μM IBV target was added to 198 μL of viral lysis buffer and loaded into a Chip Shop tank. A Drummond micropipette was used to deliver 10 μL of lysed sample through parafilm into LAMP reaction chamber. The tank was removed and the chamber was sealed.

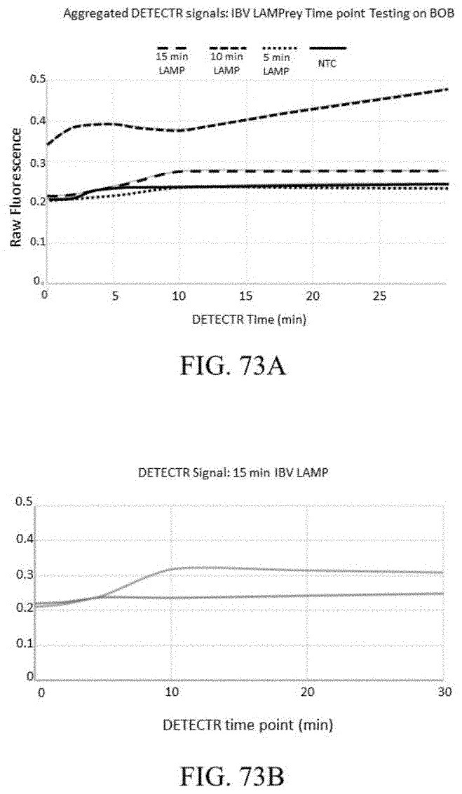

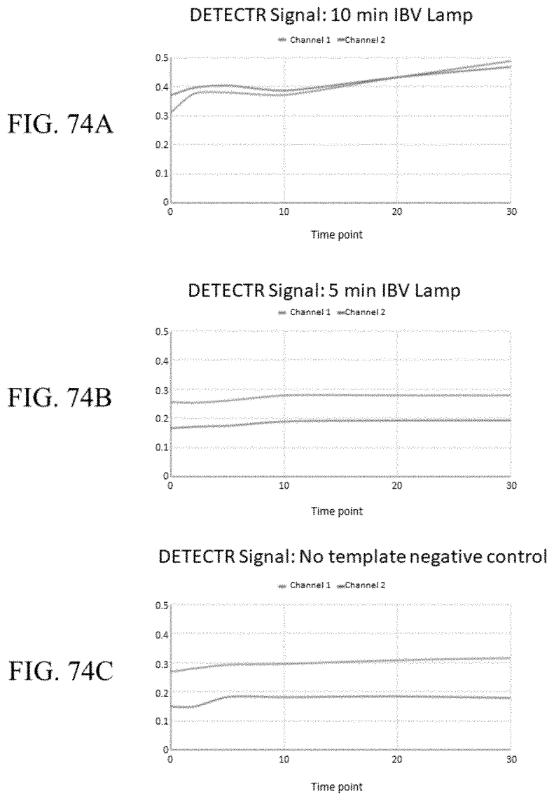

FIG. 73A, FIG. 73B, FIG. 74A, FIG. 74B, and FIG. 74C show the photodiode measurements for an influenza B DETECTR reaction in a microfluidic cartridge. 10 minutes of amplification time resulted in an increase in signal above the background (this was observed visually as well). 5 minutes of amplification time did not result in a visible increase in signal. FIG. 73A shows line graphs titled Aggregated DETECTR signals: IBV LAMPrey Time point Testing on BOB. The x-axis shows time in minutes ranging from 0 to 25 in increments of 5. The y-axis shows raw fluorescence ranging from 0 to 0.5 in increments of 0.1. The 3 lines near the middle are 15 min LAMP, 5 min LAMP, and NTC with the topmost line of the 3 liens being 15 min lamp. The topmost line in the graph is 10 min LAMP. FIG. 73B shows line graphs titled DETECTR Signal: 15 min IBV LAMP. The x-axis shows time in minutes ranging from 0 to 30 in increments of 10. The y-axis shows raw fluorescence ranging from 0 to 0.5 in increments of 0.1. The two lines near the middle are Channel 1 and Channel 2, with the Channel 1 line being higher.

### Example 32

### Device for Automating Sequential Amplification and CRISPR Reactions

This example describes a device capable of performing multiple amplification and CRISPR reactions on a sample. The device is capable of dividing a sample to perform multiple, distinct sequences of amplification and CRISPR reactions on different aliquots of a single input sample. The device houses a microfluidic chip containing multiple compartments for storing reagents and reacting the sample. The device is configured to detect signals produced from the CRISPR reactions (e.g., optical signals), and thus facilitates a plurality of measurements from a single sample input. A possible application of the device is to perform separate series of amplification and CRISPR reactions to assay a single biological sample for a large number of viruses.

A schematic for the microfluidic chip is depicted in FIG. 75. Upon insertion into the device, a biological sample will be transported a first compartment (V1), where the sample can be combined with a variety of solutions (e.g., lysis buffer) depending on the type of sample and the number and types of assays to be performed. In some assays, V1 will be preloaded with a dilution buffer prior to the sample being loaded. The device can move (e.g., via a pump) a controlled quantity of the sample (e.g., 5 μl) from the first compartment into a second compartment (V2), where it can be mixed with amplification reagents from P1. The device controls the temperature of V2 to facilitate an amplification reaction. The device transports portions of the amplification product from V2 to either V3 or V4, where the sample is mixed with reagents for CRISPR reactions. Sample from V3 and V4 can be transported to waste compartments (V5 and V6, respectively).

A depiction of the device is provided in FIG. 76. The device is configured to hold the microfluidic chip 101 below a sample inlet port 102. The inlet port contains a projection 103 (e.g., a pneumatically driven needle) that can pull a sample into a first compartment in the microfluidic chip 104. The microfluidic chip can be removed and replaced, and is held over temperature control elements 105 that modulate the temperature within compartments in the microfluidic chip. The device contains a diode array 106 configured to measure absorbance and fluorescence from multiple microfluidic chip compartments. The device utilizes batteries 107 as a power source.

### Example 33

### Flu DETECTR Reaction with Dual Amplification, Viral Lysis Buffer System

This example describes an assay for detecting flu viral nucleic acids. The assay is a combination of ambient temperature RT-LAMP amplification and guide nucleic acid driven, programmable nuclease-based detection. LAMP protocols often require strict operating temperatures that are unfeasible for implementation in devices that perform multiple types of reactions. For example, the high temperatures required for some amplification reactions can damage reagents for CRISPR reactions. This example discloses activators for LAMP amplification that are operable at a range of temperatures, including ambient temperatures, that are more suitable for implementation within a device. This example also provides viral lysis buffers containing the LAMP activators, enabling concurrent lysis and amplification upon input of a sample, such as a swab containing nucleic acids associated with the flu.

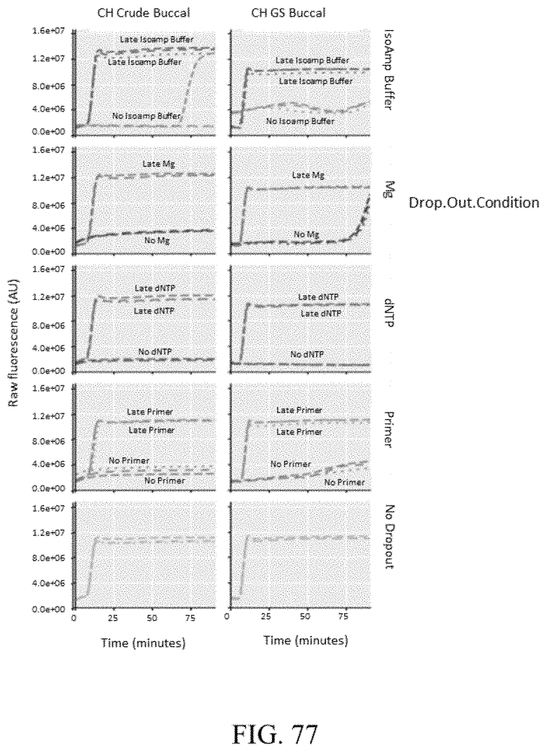

A variety of potential LAMP activators were tested for LAMP activating capacity and viral lysis buffer compatibility. LAMP activating capacity was evaluated by performing dual LAMP-DETECTR assays in the absence of individual LAMP activators. In these assays, LAMP was performed with three out of four of a buffering agent, an activator, dNTPs, and primer. The DETECTR reactions were performed on buccal swab samples with SEQ ID NO: 28 and the guide nucleic acid (targeting HERC2) given in TABLE 10 below. The DETECTR reactions were monitored by fluorescence over 90 minutes. A separate control assay was performed with all four reagents present during the LAMP amplification. As shown in FIG. 77, the LAMP reactions were inhibited by the absence of any of the four reagents. Different extraction conditions are shown in the two columns. The left column shows crude lysis, and the right column shows a standard commercial extraction method.

TABLE 10

| Species | SEQ ID NO | Sequence |
|---|---|---|
| Guide | SEQ ID NO: | UAAUUUCUACUAAGUGUAGAUAGCAUUAAGUGUC |
| nucleic acid | 270 | AAGUUCU |
| Reporter | | /5 Alex594N/TTATTATT/3IAbRQSp/ |
| nucleic acid | | |
| Target | SEQ ID NO: | TAACTCTGAAAACATTTCTAGTCTTGTAATCAACAT |
| nucleic acid | 271 | CAGGGTAAAAATCATGTGTTAATACAAAGGTACAG |
| | | GAACAAAGAATTTGTTCTTCATGGCTCTCTGTGTCT |
| | | GATCCAAGAGGCGAGGCCAGTTTCATTTGAGCATTA |
| | | AGTGTCAAGTTCTGCACGCTATCATCATCAGGGGCC |
| | | GAGGCTTCTCTTTGTTTTTAATTAATTGTTTTTAACT |
| | | GTGAGTTTATATACACTTGAAGCA |

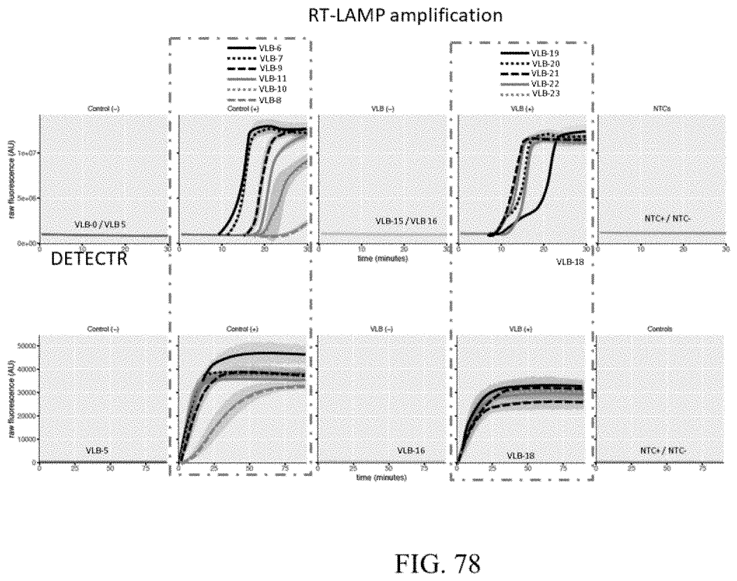

FIG. 78 shows the results of dual LAMP-DETECTR assays targeting a flu nucleic acid. Panels in the first and third columns show negative results for LAMP reactions lacking an activator. Samples were detected with a gRNA corresponding to SEQ ID NO: 377 (UAAUUUCUACUAAGUGUAGAUAGCUGCUCGAAUUGGCUUUG R1463) targeted to a target sequence corresponding to SEQ ID NO: 378 (AGCAGAAGCAGAGGATTTGTTTAGTCACTGGCAAACAGGAAAAAAAAATGGCG GACAACAACATGACCACAACACAAATTGAGGTGGGTCCGGGAGCAACCAATGC CACCATAAACTTTGAAGCAGGAATTCTGGAGTGCTATGAAAGGCTTTCATGGCA AAGGGCCCTTGACTACCCTGGTCAAGACCGCCTAAACAGACTAAAGAGAAAATT AGAGTCAAGAATAAAGACTCACAACAAAAGTGAGCCTGAAAGTAAAAGGATGT CTCTTGAAGAGAGAAAAGCAATTGGAGTAAAAATGATGAAAGTACTCCTATTTA TGAATCCGTCTGCTGGAATTGAAGGGTTTGAGCCATACT). Panels in the second and fourth column show results for LAMP reactions performed in buffer (panel in second column) and viral lysis buffer (panel in fourth column) in the presence of an activator.

### Example 34

### Multi-Chamber Injection-Molded Cartridge for Parallel Amplification and CRISPR Reactions

This example describes a fully integrated device capable of performing multiple amplification and DETECTR reactions on one input sample. The device contains an inlet port for inserting a sample, an injection-molded cartridge containing reagents for the amplification and DETECTR reactions, a fluidic system for partitioning a sample for multiple reactions, detection components for analyzing the reactions, and hardware for processing the reactions. Inserting a sample into the inlet port seals the sample within the device, preventing the sample and surrounding environment from contamination.

FIG. 79 panel (a) shows an injection-molded cartridge. The injection-molded cartridge contains an inlet port 101 for inserting a sample. The bottom of the inlet port is narrow, allowing a swab to snap and seal into place upon insertion. The top of the inlet port is attached to a cap 102 that is configured to hermetically seal the inlet port. The injection-molded cartridge contains fluidic channels 103 (e.g., microfluidic channels) through which samples and reagents can flow, including a metering channel 103a that apportions portions of the sample with defined volumes. The channels are interconnected by locations that can accommodate pumps (e.g., peristaltic pumps, hydraulic pumps, ports connecting to pneumatic pump manifolds, etc.) and switchable vales 104 that direct and meter the fluid flow. Some channels contain or terminate in compartments for reactions 105. The cartridge contains an array of reagent storage compartments 106 coupled to ports 107 for transporting the reagents throughout the fluidic channels and reaction compartments. The injection-molded cartridge is constructed from two pieces 108 & 109 that connect to hermetically seal reagents stored within the cartridge. The injection-molded cartridge chambers further comprise laser bonded sealing layers.

FIG. 79 panel (b) shows a device capable of housing the injection-molded cartridge. The device contains top 110 and bottom 111 platforms designed to hold the injection-molded cartridge firmly in place. The device contains an array of pumps and switchable valves 112 that control hydraulics within the injection-molded cartridge, and heating elements 113 that modulate temperature within the injection-molded cartridge. A fluorimeter 114 housed within the device is capable of measuring fluorescence from detection chambers in the injection-molded cartridge. A computing device 115 controls the fluorimeter, motors, and heating elements within the device.

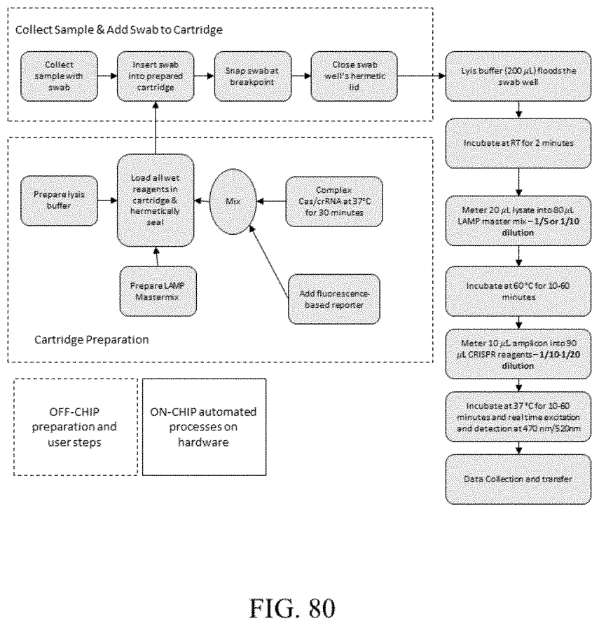

FIG. 80 shows an assay method utilizing the device that minimizes user input. The method includes off-chip preparation steps that require user input and on-chip automated processes that are controlled by the device. The injection-molded cartridge contains multiple compartments for reagents. Prior to use in an assay, compartments need to be filled with lysis buffer, amplification reagents, and DETECTR reagents including a fluorescence-based reporter, a programmable nuclease, and a guide nucleic acid. The injection-molded cartridge has multiple compartments capable of storing multiple, different sets of amplification and DETECTR reagents (e.g., amplification and DETECTR reagents with different target sequences). Prior to loading, the programmable nuclease and guide nucleic acid need to be incubated at 37° C. for 30 minutes. Once the reagents have been loaded, the injection-molded cartridge can be hermetically sealed, and then loaded into the device. The injection-molded cartridge may be reloadable, or may come pre-loaded with reagents. In such a case, the device can mix and preheat the guide nucleic acid and programmable nuclease prior to performing the DETECTR reaction.

The injection-molded cartridge contains an inlet port for sample insertion. Once the injection-molded cartridge has been prepared with reagents and sealed, a sample can be collected on a swab and inserted into the inlet port. The inlet port is configured so that a swab can be snapped at a break point within the inlet port to fix the sample within the injection-molded cartridge. Once a sample has been fixed in the injection-molded cartridge, the inlet port can be sealed with a hermetic lid.

The sealed injection-molded cartridge (loaded with reagents and a sample) can be inserted into the device, which automates sample preparation and analysis. The device first incubates the sample with 200 μl lysis buffer for 2 minutes. The device meters 20 μl aliquots of the sample into 80 or 180 μl LAMP mastermix for isothermal amplification at 60° C. for 10-60 minutes. 10 μl aliquots of the resulting amplicon are metered into 90 or 190 μl solutions containing DETECTR reagents, and incubated at 37° C. concurrent with real-time excitation and detection at 470 nm and 520 nm. The device collects and transfers this data (e.g., as a radio signal) to computing devices for analysis. The device can perform and detect a large number of sequential and parallel amplification and detection reactions targeting different nucleic acid sequences on a single sample.

FIG. 81 shows optical assemblies for the device. FIG. 81 panel (a) shows an array of diodes 116 that can produce 470 nm light and detect 520 nm or 594 nm light to excite and detect reporter molecules, respectively. FIG. 81 panel (b) shows the diode array with the amber and blue LEDs illuminated. FIG. 81 panel (c) shows an injection molded cartridge illuminated by the diode array.

FIG. 82 shows a possible design for an injection-molded cartridge. The injection-molded cartridge contains a sample chamber 117 for collecting and then mixing a sample with up to 400 μl of buffer. The sample chamber contains a pump, and is connected through a rotary valve to a series of fluidic channels 118 (e.g., microfluidic channels) which partition the sample into multiple amplification chambers 119. A metering valve within the rotary valve at the exit of the sample chamber dispenses 20 μl aliquots from the sample chamber via into the fluidic channels per rotation. The amplification chambers are coupled to amplification reagent chambers (which contain reagents for the amplification reactions) 120 through resistance channels 118b, which are each configured with a pump and a valve that control the flow of stored reagents into the amplification chambers. The back end of each amplification chamber is connected to a valve that meters flow through a second series of fluidic channels 121 into a series of detection chambers 122. The detection chambers are coupled to detection reagent chambers (which store reagents for the detection reactions) 123 through resistance channels 118b, which are each configured with a pump and a valve that control the flow of stored reagents into the detection chambers. This injection-molded cartridge contains one sample chamber, 5 amplification chambers, and 10 detection chambers.

### Example 35

### Injection-Molded Cartridge Design for Performing Multiple Amplification and DETECTR Reactions on a Single Sample

This example provides a design for an injection molded cartridge capable of partitioning a sample for separate amplification and DETECTR reactions. The injection-molded cartridge is designed to collect samples from swabs (e.g., buccal swabs). The combinations of distinct amplification and DETECTR reactions allow the sample to be assayed for multiple sequences. For example, the 8 DETECTR reaction could be used to query for 8 separate viruses or 7 viruses and an internal control. The injection-molded cartridge is designed to fit within a device that automates sample and reagent movement, heating, and detection.

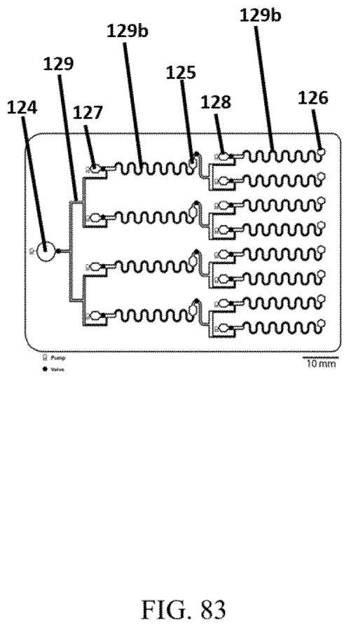

FIG. 83 shows an injection-molded cartridge design with 1 sample chamber 124, 4 amplification chambers 125, and 8 detection chambers 126. Each amplification chamber and detection chamber is connected by a resistance channel 129b to one amplification reagent chamber 127 or one detection reagent chamber 128, respectively. Each series of chambers is connected by fluidic channels 129 as shown in FIG. 83. The fluidic channels connecting the sample chamber to the amplification chambers are between 300 μm and 1 mm in width.

FIG. 84 shows an alternate design for the injection-molded cartridge in FIG. 83, with an lysis reagent chamber 130 connected to the sample chamber 124. A valve (v1) mediates flow between the lysis reagent chamber and sample chamber. V1-V18 correspond to valves to control flow between chambers.

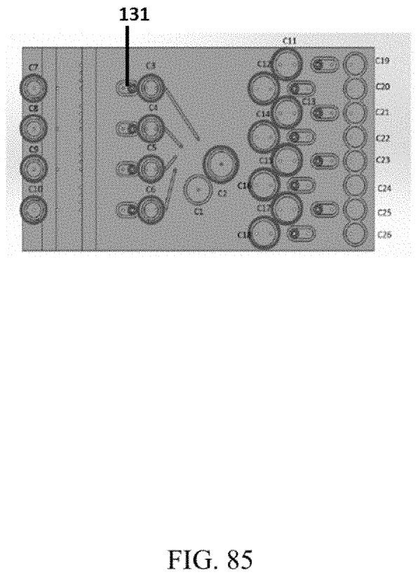

FIG. 85 shows a design for the top of an injection-molded cartridge similar to the one depicted in FIG. 84. The injection-molded cartridge can be connected to a manifold for pressure-driven flow. The labeled chambers C1 and C2 correspond to the lysis reagent chamber and sample chamber in FIG. 84. The labeled chambers C3-C6 correspond to the amplification reagent chambers in FIG. 84. The labeled chambers C7-C10 correspond to the amplification chambers in FIG. 84. The labeled chambers C11-C18 correspond to the detection reagent chambers in FIG. 84. The labeled chambers C19-C26 correspond to the detection chambers in FIG. 84. In this design, the sample chamber and the lysis reagent chamber are located near the center of the injection-molded cartridge. The valves controlling flow from C3-C6 and C11-C18 can be controlled 131 from the top of the injection molded cartridge. The detection reagent chambers and detection chambers are also spaced further from the amplification chambers to further isolate detection reagents (e.g., reagents for CRISPR reactions) from the temperatures of the amplification reactions, as in some cases, detection reagents (e.g., CRISPR reaction reagents) aren't stable at the temperatures required for amplification reactions.

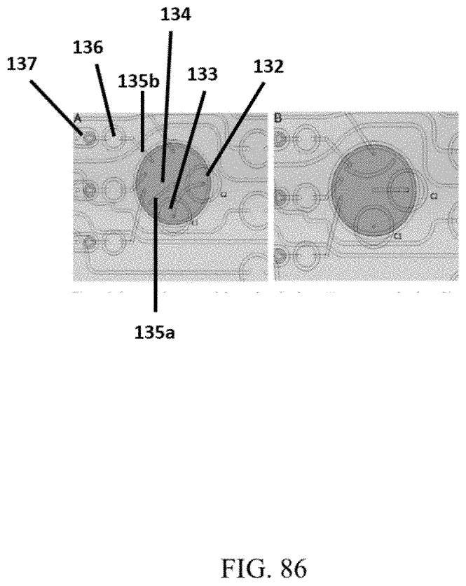

FIG. 86 shows a design for a portion of an injected molded cartridge containing a sample chamber 132 and a lysis reagent chamber 133 that are connected by a rotary valve 134, which is sealed with laser bonded clear polycarbonate. A swab containing a sample can be inserted into the sample chamber. Lysis buffer can be pumped from the lysis reagent chamber to the sample chamber by a partial rotation of a rotary valve 134. The rotary valve contains a metering channel 135a that can transfer a defined volume of liquid from the sample compartment into a channel 135b leading to an amplification chamber 136. Thus, the device is capable of sequentially transfer aliquots from the sample chamber to each of the individual amplification chambers. Flow out of each amplification chamber is controlled by a valve 137, which is connected to a vent. Panel A shows the rotary valve connecting the lysis reagent chamber to the sample chamber. Panel B shows the injection-molded cartridge after the rotary valve has been partially rotated (relative to panel A).

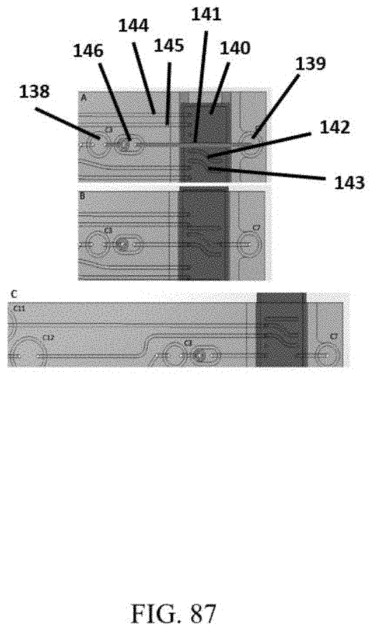

FIG. 87 shows a design for a portion of an injected molded cartridge containing an amplification reagent chamber 138 and an amplification chamber 139 connected by a slider valve 140. The slider valve has four positions, a first position for delivering fluid into the amplification chambers (shown in panel A) through a first metering channel 141, two positions for metering fluid out of the amplification chamber and into metering channels 142 & 143 (one of these two positions is depicted in panel B), and a fourth position in which the metering channels connect to fluidic channels 144 & 145 leading to separate detection chambers (shown in panel C). A valve 146 in between the amplification reagent chamber and amplification chamber controls flow between the two chambers when the slider valve is in the first of the four positions (shown in panel A).

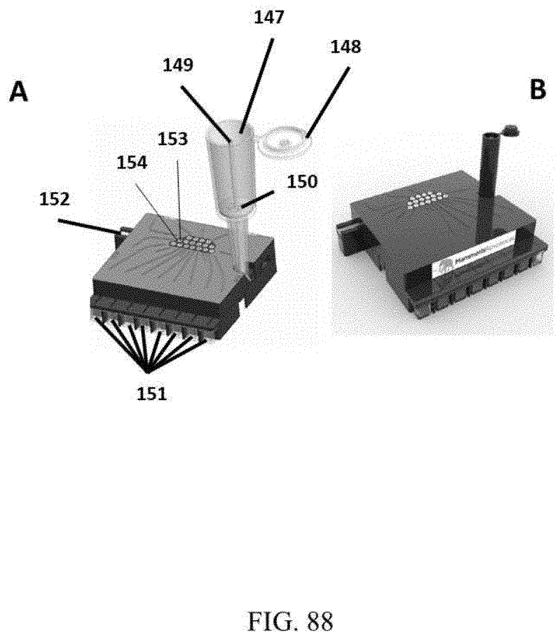

FIG. 88 shows a design for an injection-molded cartridge with a plastic shell. The design includes a sample inlet port 147 leading to the sample chamber with a hermetically sealing cap 148. The sample inlet port is designed to accommodate a swab 149. Lysis buffer can be loaded into the top of the sample inlet port prior to insertion of the swab. Insertion of the swab breaks a seal, allowing the lysis buffer to flow through the bottom of the sample inlet port and into the sample chamber. Once inserted, the swab locks in place against a set of plastic projections 150, minimizing sample contamination. Closing the cap over the sample inlet port further protects against contamination. The design is rectangular so that the detection chambers 151 have flat faces for excitation light to pass through during fluorescence detection. The slider valve 152 that meters flow through the amplification chambers can be seen near the back of the injection-molded cartridge. The top of the injection-molded cartridge contains multiple ports 153 terminating in O-rings 154 allow the cartridge to connect to a pneumatic pumping manifold that can apply pressure to individual cartridge chambers. Panel A depicts a design for an injection-molded cartridge. Panel B is a picture of a functional model of an injection-molded cartridge similar to the one shown in panel A. The injection-molded cartridge in panel C differs from the injection molded cartridges in panel A by its sample inlet port, which lacks the breakable seal and projections for holding a swab.

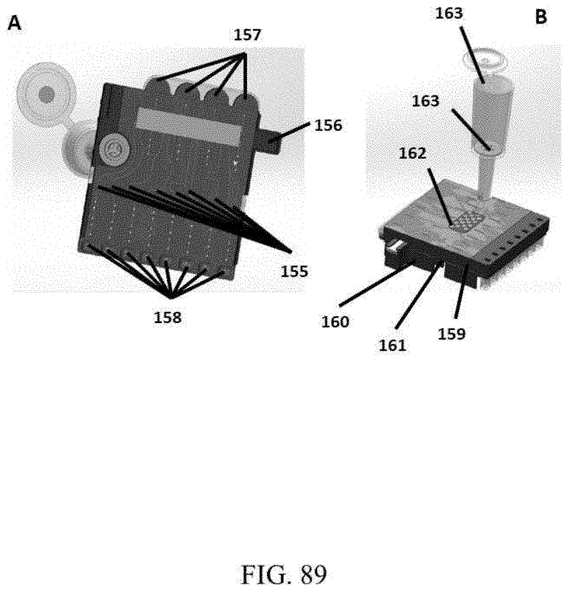

FIG. 89 panel A shows a bottom view of an injection-molded cartridge design. This design features wide, flat reagent chambers (e.g., amplification reagent chambers) to enable rapid heating and fast fluid mixing by pumping the fluids back and forth into and out of reagent chambers, rather than sequentially flowing different solutions into a single chamber. The short cartridge height allows a heater to wrap around the reaction compartments. The lengths of the channels 155 that connect the same types of chambers to provide equal fluidic resistance when used for mixing. The bottom of the sliding valve 156, amplification reagent chambers 157 and detection chambers 158 can be seen from the bottom of the cartridge. Panel B shows a top view of the injection-molded chip. Top 159 and bottom 160 plastic casing pieces form a hermetic seal around the injection molded chip. Interlocking clips 161 on the plastic casing pieces facilitate easy assembly into a single unit. A series of O-ring topped ports 162 allow the injection molded cartridge to couple to a pneumatic pumping manifold that can control flow throughout the injection-molded cartridge. A sample inlet port 163 contains a top chamber stoppered by a breakable seal 164.

### Example 36

### Injection-Molded Cartridge Capable of Performing Parallel Amplification and CRISPR Reactions on a Single Sample

This example describes an injection-molded cartridge designed to perform multiple amplification and CRISPR reactions on a single sample. This cartridge has 4 amplification chambers and 8 detection chambers. A single sample will first be diluted in a sample chamber, and then be partitioned between the four amplification chambers. The amplification products from each amplification chamber will be partitioned to two separate detection chambers. Each amplification chamber is transparent so as to allow optical (e.g., fluorescent) monitoring of the CRISPR (e.g., DETECTR) reactions. Each amplification and detection chamber is connected to a unique reagent storage chamber (e.g., an amplification reagent chamber). Some chambers can be loaded with identical reagents, or each chamber can be loaded with different reagents (e.g., amplification reagents and DETECTR reagents targeting different sequences). Thus, the injection-molded cartridge is capable of performing up to 8 unique sequences of amplification and CRISPR reactions on a single input sample.

The injection-molded cartridge is configured to insert into a device capable of controlling sample partition, reagent loading, heating and detection within the cartridge. The cartridge contains multiple valves along with a pneumatic delivery manifold, which collectively allow a device to control the flow, pressure, and temperature in the chambers and fluidic channels within the device. The device can also be equipped with an optical detector (e.g., a fluorimeter) capable of measuring the components of the detection chambers.

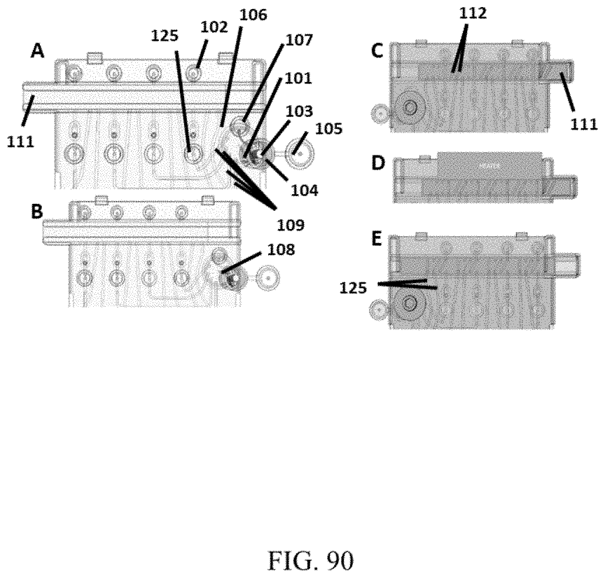

FIG. 90 shows designs for a portion of the injection-molded cartridge containing the sample chamber 101 and amplification chambers 102. Panels A & B provide top-down views, while panels C through E show the injection-molded cartridge from the bottom. As shown in panel A, a swab 103 containing the sample to be analyzed can be inserted into a sample inlet port 104. The sample inlet port has a hermetically sealing cap 105, which seals the contents of the injection-molded cartridge from the surrounding environment. Once a sample has been inserted into the sample chamber, a rotating valve 106 can transport lysis buffer from a lysis buffer storage chamber 107 to the sample chamber. Panel A shows the rotating valve connecting the lysis buffer storage and sample chambers. Once sample lysis has completed, the rotating valve can transfer 20 μl aliquots of the sample into a metering channel 108 that can be rotated to deliver sample into microfluidic channels 109 leading to the four amplification reagent chambers 110. Panel B shows the rotating valve positioned to connect the metering channel with the sample chamber.

As shown from the bottom-up view depicted in panel C, the contents of the amplification reagent chambers can flow into the amplification chambers 101. Mixing is performed by moving the contents back and forth between the two chambers. Once mixing is complete, the samples are completely transferred into the amplification chambers and incubated for a controlled period of time. As is shown in panel D, the injection-molded cartridge can be situated over a heating element within the control-device, thus allowing temperature control during the amplification during.

The direction of flow into and out of the amplification chambers is mediated by a slider valve 111. Panel C depicts the slider valve in a first position that connects each amplification reagent chamber to an amplification chamber. Once the amplification reaction is complete, the panel can slide to second and third positions (one of which is depicted in panel E) that allow sample to move from the amplification chambers into metering channels 112. The slider is then capable of adopting a fourth position in which the metering channels overlap with channels 113 that lead to the detection reagent chambers. Thus, the sample is divided into 8 separate components following amplification.

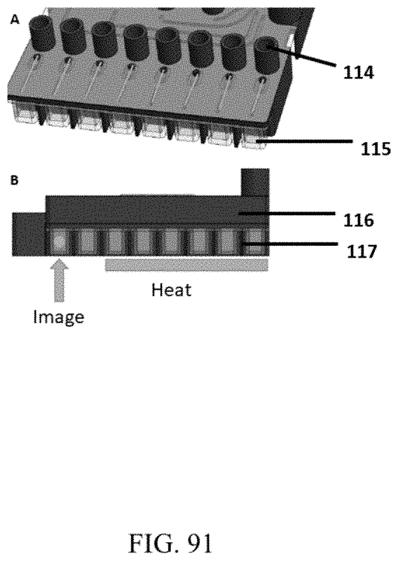

FIG. 91 panel A provides a design for the portion of the injection-molded cartridge containing the detection reagent chambers and detection chambers. Following amplification, the sample flows from the amplification chambers and into the detection reagent chambers 114. The sample then flows from the detection reagent chambers and cascades downwards into the detection chambers 115. The injection-molded cartridge connects to a plastic cover piece, which fits over the top of the cartridge and seals its chambers. Panel B shows the injection-molded cartridge with the plastic cover piece 116. As is shown in the side-on view of panel B, the detection chambers have flat, transparent surfaces enabling fluorescence excitation and detection. The detection chambers are situated over a second heater in the control device capable of elevating the temperatures of the detection chambers. Black bosses between the detection chambers minimizes light contamination between chambers, thus improving the accuracy and sensitivity of optical experiments (e.g., luminescence detection, fluorescence, etc.).

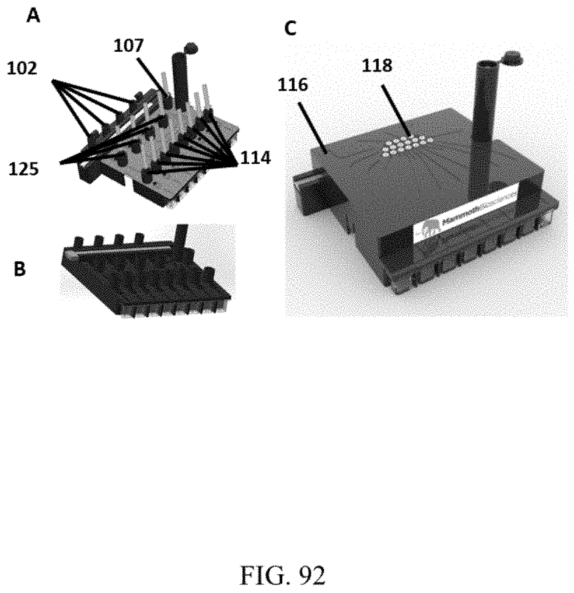

FIG. 92 panels A and B provide full views of the injection molded cartridge. The amplification chambers 102, lysis buffer storage chamber 107, amplification reagent chambers 110, and detection reagent chambers 114 are open, and can be loaded with solutions and reagents. Once desired reagents are loaded into the device, a plastic cover piece can be attached to the injection-molded cartridge, sealing the chambers and fluidic channels within the device. Panel C shows a picture of a working physical model of the injection molded cartridge with the plastic cover piece 116 attached. The plastic cover piece contains an array of O-ring topped inlet ports 118 that can connect to a pneumatic manifold capable of directing flow throughout the chambers and fluidic channels within the injection-molded cartridge. The total dimensions of the cartridge are 92 mm×80 mm×52.5 mm including the height of the sample inlet port, and 92 mm×80 mm×19.5 mm excluding the sample inlet port. A retaining ring forms a seal between the injection-molded cartridge and inlet port, which are otherwise distinct and separable.

### Example 37

### Diode Array for Excitation and Detection of Fluorescent Detection from an Injection-Molded Cartridge

This example covers a detection scheme for fluorescent read-out DETECTR reactions in a multi-chamber cartridge. The cartridge is designed to perform separate DETECTR reactions on separate portions of a sample that have undergone amplification. FIG. 93 shows an injection-molded cartridge 101 housed in a device 102 containing a diode array capable of detecting light from each of the chambers and white light emitting diodes 103 positioned to illuminate the chambers. The injection-molded cartridge has 8 detection chambers 104. The four leftmost (orange) detection chambers contain the dye ATTO 594, and the four rightmost chambers contain the dye ATTO 488. The front faces (pointing out of the device opening) of the detection chambers that contain the 594 dye are coated with an orange gel filter. The front faces of the detection chambers that contain the 488 dye are coated with a yellow filter. White lights illuminate the detection chambers from the side, exciting fluorescent dyes within the detection chambers. The sides of the detection chambers facing the white lights may be coated. with optical filters or color-absorbent gels. The device contains diodes that detect light emitted from the detection chambers, thus allowing the device to monitor DETECTR reactions with fluorescent reporters.

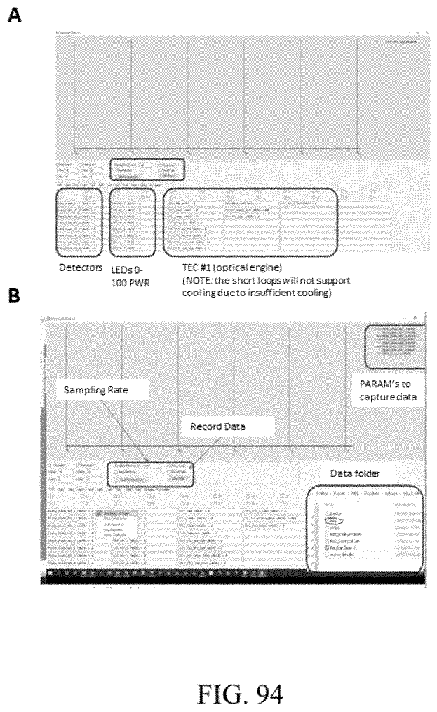

FIG. 94 panels A and B show a graphic user interface for controlling the white lights, detector diodes, and for monitoring data collected on the detector diodes. The graphic user interface allows the user to set temperature shutoff points (e.g., configure a detector diode to shut off if its temperature exceeds 50° C.), the bias voltage or current through the diodes, and the sampling rate (e.g., 100 Hz) on each detector diode. The graphic can display fluorescence readout data from each detector diode.

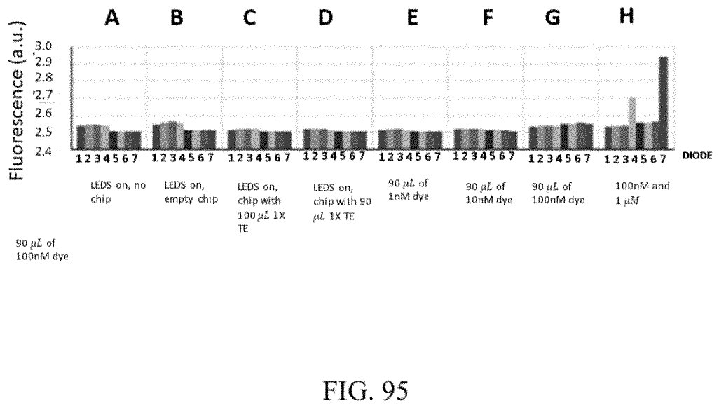

FIG. 95 shows the results of a calibration test for the diode array. Each set of 8 datapoints corresponds to the data collected by the 8 detector diodes in a single test. Data set A was collected without an injection molded cartridge in the device. Data sets B-H were collected with an empty injection molded cartridge in the device. Data set B was collected on the empty cartridge. Data sets C and D were collected with the cartridge containing buffer but no dye. Data sets E, F and G were collected with the cartridge containing 1 nM, 10 nM and 100 nM dye, with diodes 1-4 collecting on wells containing ATTO 488 and wells 5-8 containing ATTO 594. Data set H was collected with 100 nM ATTO 488 in wells 1-3, 1 μM ATTO 488 in well 4, 100 nM ATTO 594 in wells 5-7, and 1 μM ATTO 594 in well 8. FIG. 95 shows bar graphs in 8 sections designated as A, B, C, D, E, F, G, and H. Section 1 is LEDS on, no chip, Section B is LEDS on, empty chip, Section C is LEDS on, chip with 100 ul 1×TE, Section D is LEDS on, chip with 90 ul 1×TE, Section E is 90 ul of 1 nM dye, Section F is 90 ul of 10 nM dye, Section G is 90 ul of 100 nM dye, and Section H is 100 nM and 1 uM. Within each section are 7 bars, which from left to right are DIODE 1, DIODE 2, DIODE 3, DIODE 4, DIODE 5, DIODE 6, DIODE 7, and DIODE 8. The y-axis shows fluorescence in arbitrary units (a.u.) ranging from 2.4 to 3.0 in increments of 0.1.

### Example 38

### HERC2 DETECTR Assay Performed Measured with a Diode Array

This example describes a DETECTR Assay performed on the injection molded cartridge of EXAMPLE 36 using the diode array of EXAMPLE 37. The reagents for the DETECTR assays were loaded directly into the detection chambers. The assays utilized a programmable nuclease with SEQ ID NO: 28, a guide nucleic acid with SEQ ID NO: 270 targeting HERC2 G SNP allele, and a reporter nucleic acid which increased fluorescence response upon cleavage. Four wells contained 5 μM reporter, 150 nM programmable nuclease, 600 nM guide nucleic acid, and 500 μM target nucleic acid. Two wells contained 5 μM reporter, 150 nM programmable nucleic acid, 600 nM guide nucleic, and no target. Two wells contained only buffer. The reporters contained either ATTO 488 or ATTO 594.

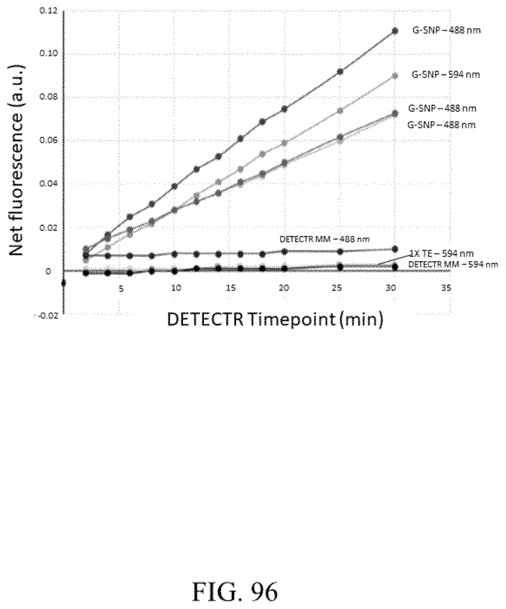

FIG. 96 shows fluorescence traces from the 8 detection chambers measured by an 8 diode detector array. The detection chambers containing a reporter, programmable nuclease, guide nucleic acid, and target nucleic acid provided fluorescence responses that increased linearly with time. The detection chambers containing DETECTR reagents but lacking the target nucleic acid and the detection chambers containing only buffer did not display increases in fluorescence. Thus, the detection chambers with active transcollateral reporter cleavage were distinguishable by fluorescence. FIG. 96 shows line graphs with the x-axis showing the DETECTR timepoint in minutes ranging from 0 to 35 in increments of 5 and the y-axis showing the net fluorescence in arbitrary units (a.u.) ranging from −0.02 to 0.12 in increments of 0.02. The four lines linearly increasing are from left/highest to right/lowest are G-SNP-488 nm, G-SNP-594 nm, G-SNP-488 nm, and G-SNP-488 nm. The last two in the prior list are nearly overlapping. The higher flat line near the bottom corresponds to DETECTR MM-488 nm. The lower flat lines at the bottom correspond to DETECTR MM-594 nm and 1×TE-594 nm.

FIG. 97 shows an image of the detection chambers 30 minutes after DETECTR reagent addition. Detection chambers 1, 4, 5, and 8 contained the target nucleic acid, and are visibly brighter than the remaining detection chambers.

### Example 39

### Detection of Coronavirus Variants Using a Reverse Transcriptase PCR DETECTR Assay

Reverse Transcriptase PCR DETECTR reactions can be used for the detection of a SARS-CoV-2 variant, particularly the United Kingdom (UK) variant known as 20B/501Y.V1, VOC 202012/01, or B.1.1.7 lineage, or the South African variant known as: 20C/501Y.V2 or B.1.351 lineage. See www.cdc.gov/coronavirus/2019-ncov/more/science-and-research/scientific-brief-emerging-variants.html. The genetic characteristics of these variants are discussed in Leung et. al, Early transmissibility assessment of the N501Y mutant strains of SARS-CoV-2 in the United Kingdom, October to November 2020, Euro Surveill. 2021; 26(1) and in Tegally et. al., Emergence and rapid spread of a new severe acute respiratory syndrome-related coronavirus 2 (SARS-CoV-2) lineage with multiple spike mutations in South Africa, MedRxiv 2020.12.21. A sample containing a target nucleic acid corresponding to the UK or South African variant can beamplified using one or more of the primers listed in Table 12, and a mutation in the nucleic acid characteristic of the variant can be detected using a fluorescent assay leveraging Cas12M08, a variant within the Cas12 family having SEQ ID NO: 28, and one of the gRNAs described in Table 14. RT-LAMP, which is described in the examples herein, may alternatively be used for the amplification. One skilled in the art will recognize that RT-LAMP methods may alternatively be used for the amplification method reaction with appropriate modifications to the primers.

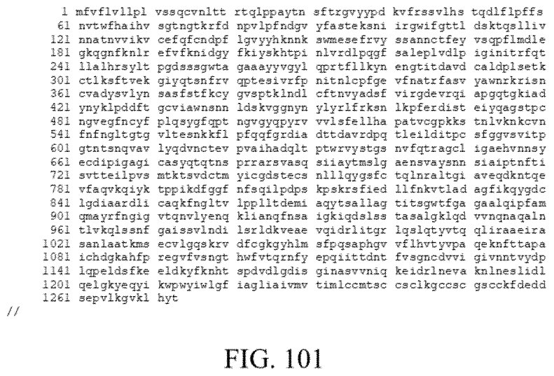

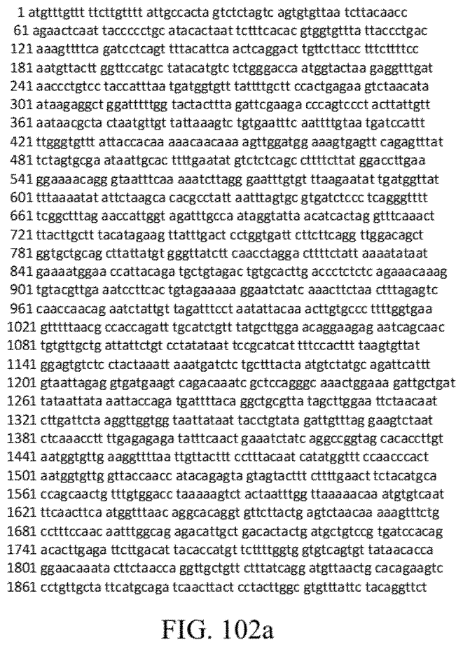

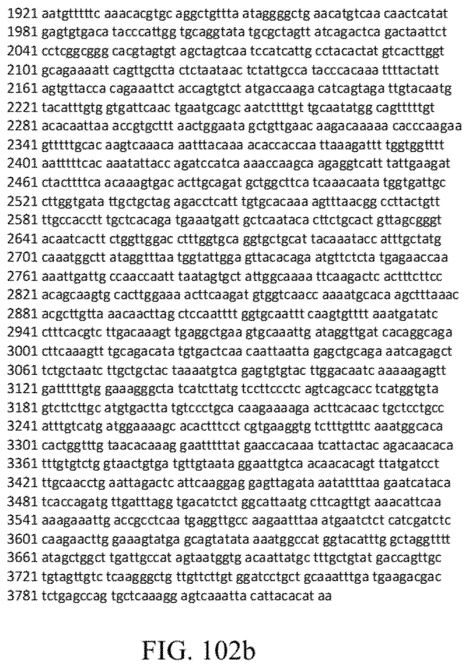

Table 11 lists certain mutations in the Spike gene characterizing the UK and South African variants, one or more of which can be selected as targets for the RT-PCR DETECTR reaction. The amino acid sequence of the Surface Glycoprotein (GenBank Ref QHD43416.1) encoded by the Spike (“S”) gene (GenBank Ref MN908947.3:21563..25384) is provided in FIG. 101. The nucleotide sequence of the Spike gene is provided in FIGS. 102A and 102B. Other mutations that can be used to differentiate between the strains of interest are located in other regions of the SARS-CoV-2 genome. Regarding Tables 11-14, amino acid mutations are described in the form of: [wild type amino acid][amino acid number][mutant amino acid] relative to the sequence depicted in FIG. 101. The lowercase nucleotides in parenthesis correspond to the wild-type nucleotides and the uppercase nucleotides in parenthesis correspond to the mutant nucleotides comprising the mutation (i.e., encoding the mutant amino acid). Additionally, “xxx” refers to an unknown SNP. SARS-CoV-2 target sequences have been obtained using all available genomes available from GISAID.

TABLE 11
Genetic Changes Characterizing the
UK and South African Variants.

| “UK” Variant Mutations | “South African” Variant Mutations |
|---|---|
| del69/del70 (68-70 ata cat gtc) | L18F (ctt/xxx) |
| del144 (143-144 gtt tat) | D80A (gat/GCT) |
| N501Y (aat/TAT) | D215G (gat/GGT) |
| A570D (gct/GMT) | del242-244 (240-243 actttacttgct |
| | (SEQ ID NO: 379)) |
| P681H (Nigerian)(cct/CAT) | R246I (aga/ATA) |
| T716H (aca/ATA) | K417N (aag/AAT) |
| D1118H (gac/CAC) | E484K (gaa/AAA) |
| | N501Y (aat/TAT) |
| | D614G (gat/GGT) |
| | A701V (gca/GTA) (edited) |

Any of the regions of the Spike gene comprising the groups of mutations detailed in Table 12 may be selected as an amplicon. Table 12 lists mutations present in the Spike gene (reference name MN908947.3). The Spike region is the most variable region of the viral genome and is a major region for current SARS-CoV-2 vaccine design. Amplicons may be selected comprising the start and stop nucleotides (nt) given in the 2nd and 3rd columns of Table 12, respectively. The three columns to the right detail whether the mutations are found in the UK, South African and/or both variants. Further, Table 13 details gene fragments of the South African and UK variants comprising the various mutations detailed in Table 11. Column 2 of Table 13 details the mutation comprised in the gene fragments.

TABLE 12
Exemplary mutations for combined strains of
the Spike gene (reference name MN908947.3).

| | | | | Found in the | |
|---|---|---|---|---|---|
| | | | Found in | South African | |
| Mutation | Start | Stop | the UK | Variant | Found in |
| Name | position | position | Variant | (501Y.V2) | Both |
| E484K, | 23012 | 23270 | A570D | E484 | N501Y |
| N501Y, | | | | | |
| A570D | | | | | |
| P681H, | 23603 | 23708 | P681H, | A701V | |
| A701V, | | | T716H | | |
| T716H | | | | | |
| P681H, | 23603 | 23663 | P681H | A701V | |
| A701V | | | | | |
| A701V, | 23663 | 23708 | T716H | A701V | |
| T716H | | | | | |
| 69-70del, | 21764 | 21802 | 69-70del | D80A | |
| D80A | | | | | |

TABLE 13
Gene fragments of the South African and UK Strains

| | Description/ |
|---|---|
| Sequence | Notes |
| tggtgtttattaccctgacaaagttttcagatcctcagttttacattcaactcaggacttgttcttacctt | del69/70; |
| tcttttccaatgttacttggttccatgctatacatgtctctgggaccaatggtactaagaggtttgata | D80A; del144; |
| accctgtcctaccatttaatgatggtgtttattttgcttccactgagaagtctaacataataagaggct | D215G; |
| ggatttttggtactactttagattcgaagacccagtccctacttattgttaataacgctactaatgttgt | del242; R246I |
| tattaaagtctgtgaatttcaattttgtaatgatccatttttgggtgtttattaccacaaaaacaacaaa | |
| agttggatggaaagtgagttcagagtttattctagtgcgaataattgcacttttgaatatgtctctca | |
| gccttttcttatggaccttgaaggaaaacagggtaatttcaaaaatcttagggaatttgtgtttaaga | |
| atattgatggttattttaaaatatattctaagcacacgcctattaatttagtgcgtgatctccctcagg | |
| gtttttcggctttagaaccattggtagatttgccaataggtattaacatcactaggtttcaaactttact | |
| tgctttacatagaagttatttgactcctggtgattcttcttcaggttggacagctggtgctgcagc | |
| (SEQ ID NO: 380) | |
| tctgctttactaatgtctatgcagattcatttgtaattagaggtgatgaagtcagacaaatcgctcca | K417N;E484K; |
| gggcaaactggaaagattgctgattataattataaattaccagatgattttacaggctgcgttatag | N501Y |
| cttggaattctaacaatcttgattctaaggttggtggtaattataattacctgtatagattgtttaggaa | |
| gtctaatctcaaaccttttgagagagatatttcaactgaaatctatcaggccggtagcacaccttgt | |
| aatggtgttgaaggttttaattgttactttcctttacaatcatatggtttccaacccactaatggtgttg | |
| gttaccaaccatacagagtagtagtactttcttttgaacttctacatgca (SEQ ID NO: | |
| 381) | |
| ctgagtctaacaaaaagtttctgcctttccaacaatttggcagagacattgctgacactactgatgc | A570D; |
| tgtccgtgatccacagacacttgagattcttgacattacaccatgttcttttggtggtgtcagtgttat | D614G; P681H; |
| aacaccaggaacaaatacttctaaccaggttgctgttctttatcaggatgttaactgcacagaagt | A701V; T716H |
| ccctgttgctattcatgcagatcaacttactcctacttggcgtgtttattctacaggttctaatgtttttc | |
| aaacacgtgcaggctgtttaataggggctgaacatgtcaacaactcatatgagtgtgacataccc | |
| attggtgcaggtatatgcgctagttatcagactcagactaattctcctcggcgggcacgtagtgta | |
| gctagtcaatccatcattgcctacactatgtcacttggtgcagaaaattcagttgcttactctaataa | |
| ctctattgccatacccacaaattttactattagtgttaccacagaaattctaccagtgtctatgaccaa | |
| gacatcagtagattgtacaatgtacatttgtggtgattc (SEQ ID NO: 382) | |
| gacaatcaaaaagagttgatttttgtggaaagggctatcatcttatgtccttccctcagtcagcacc | D1118H |
| tcatggtgtagtcttcttgcatgtgacttatgtccctgcacaagaaaagaacttcacaactgctcct | |
| gccatttgtcatgatggaaaagcacactttcctcgtgaaggtgtctttgtttcaaatggcacacact | |
| ggtttgtaacacaaaggaatttttatgaaccacaaatcattactacagacaacacatttgtgtctggt | |
| aactgtgatgttgtaataggaattgtcaacaacacagtttatgatcctttgcaacctgaattagactc | |
| attcaaggaggagttagataaatattttaagaatcatacatcaccaga (SEQ ID NO: | |
| 383) | |
| tggtgtttattaccctgacaaagttttcagatcctcagttttacattcaactcaggacttgttcttacctt | del69/70; |
| tcttttccaatgttacttggttccatgctatctctgggaccaatggtactaagaggtttgctaaccctg | D80A; del144; |
| tcctaccatttaatgatggtgtttattttgcttccactgagaagtctaacataataagaggctggatttt | D215G; |
| tggtactactttagattcgaagacccagtccctacttattgttaataacgctactaatgttgttattaaa | del242; R246I |
| gtctgtgaatttcaattttgtaatgatccatttttgggtgtttaccacaaaaacaacaaaagttggatg | |
| gaaagtgagttcagagtttattctagtgcgaataattgcacttttgaatatgtctctcagccttttctta | |
| tggaccttgaaggaaaacagggtaatttcaaaaatcttagggaatttgtgtttaagaatattgatgg | |
| ttattttaaaatatattctaagcacacgcctattaatttagtgcgtggtctccctcagggtttttcggctt | |
| tagaaccattggtagatttgccaataggtattaacatcactaggtttcaaactttacatataagttattt | |
| gactcctggtgattcttcttcaggttggacagctggtgctgcagc (SEQ ID NO: 384) | |
| tctgctttactaatgtctatgcagattcatttgtaattagaggtgatgaagtcagacaaatcgctcca | K417N; E484K; |
| gggcaaactggaaatattgctgattataattataaattaccagatgattttacaggctgcgttatagc | N501Y |
| ttggaattctaacaatcttgattctaaggttggtggtaattataattacctgtatagattgtttaggaag | |
| tctaatctcaaaccttttgagagagatatttcaactgaaatctatcaggccggtagcacaccttgta | |
| atggtgttaaaggttttaattgttactttcctttacaatcatatggtttccaacccacttatggtgttggtt | |
| accaaccatacagagtagtagtactttcttttgaacttctacatgca (SEQ ID NO: 385) | |
| ctgagtctaacaaaaagtttctgcctttccaacaatttggcagagacattgatgacactactgatgc | A570D; |
| tgtccgtgatccacagacacttgagattcttgacattacaccatgttcttttggtggtgtcagtgttat | D614G; P681H; |
| aacaccaggaacaaatacttctaaccaggttgctgttctttatcagggtgttaactgcacagaagt | A701V; T716H |
| ccctgttgctattcatgcagatcaacttactcctacttggcgtgtttattctacaggttctaatgtttttc | |
| aaacacgtgcaggctgtttaataggggctgaacatgtcaacaactcatatgagtgtgacataccc | |
| attggtgcaggtatatgcgctagttatcagactcagactaattctcatcggcgggcacgtagtgta | |
| getagtcaatccatcattgcctacactatgtcacttggtgtagaaaattcagttgcttactctaataac | |
| tctattgccatacccataaattttactattagtgttaccacagaaattctaccagtgtctatgaccaag | |
| acatcagtagattgtacaatgtacatttgtggtgattc (SEQ ID NO: 386) | |
| gacaatcaaaaagagttgatttttgtggaaagggctatcatcttatgtccttccctcagtcagcacc | D1118H |
| tcatggtgtagtcttcttgcatgtgacttatgtccctgcacaagaaaagaacttcacaactgctcct | |
| gccatttgtcatgatggaaaagcacactttcctcgtgaaggtgtctttgtttcaaatggcacacact | |
| ggtttgtaacacaaaggaatttttatgaaccacaaatcattactacacacaacacatttgtgtctggt | |
| aactgtgatgttgtaataggaattgtcaacaacacagtttatgatcctttgcaacctgaattagactc | |
| attcaaggaggagttagataaatattttaagaatcatacatcaccaga (SEQ ID NO: | |
| 387) | |

DETECTR assays are performed using reverse-transcriptase-PCR for pre-amplification. Particularly, an extreme PCR technique in which the speed of the PCR reaction is decreased to less than 5 minutes by near-instantaneous changes in the reaction temperature is used. This rapid temperature change may be accomplished by moving the reaction between heat-zones (water baths, heat blocks, etc.) of various temperatures in a thin-walled vessel, instead of cooling or heating the entire instrument for each cycle. Alternatively, the reaction volume can be pumped between two or three heat zones to achieve this rapid thermal change and drive the PCR reaction. Additional speed increases of the PCR reaction can be achieved by increasing the primer, polymerase, and Mg2+ concentrations of the reaction. One or more of the primers described in Table 14 are used. The primers have been designed using Panel Plex (https://www.dnasoftware.com/). Table 14 provides the sequence of each primer, along with the mutations comprised in the target sequence with which they are compatible.

TABLE 14
Primers designed for the Reverse Transriptase-PCR-DETECTR assay

| Name | Sequence | Description/Note |
|---|---|---|
| M6112 triple501-v1- | CTGAAATCTATCAGGCCGGTAGCA (SEQ | E484K, N501Y, |
| F | ID NO: 388) | A570D |
| M6113 triple501-v1- | GTCAAGAATCTCAAGTGTCTGTGGAT | E484K, N501Y, |
| R | (SEQ ID NO: 389) | A570D |
| M6114 triple501-v2- | TGAAATCTATCAGGCCGGTAGCAC (SEQ | E484K, N501Y, |
| F | ID NO: 390) | A570D |
| M6115 triple501-v2- | TGAAATCTATCAGGCCGGTAGCAC (SEQ | E484K, N501Y, |
| R | ID NO: 390) | A570D |
| M6116 triple501-v3- | TCAACTGAAATCTATCAGGCCGGTA | E484K, N501Y, |
| F | (SEQ ID NO: 391) | A570D |
| M6117 triple501-v3- | ATCTCAAGTGTCTGTGGATCAC (SEQ ID | E484K, N501Y, |
| R | NO: 392) | A570D |
| M6118 triple501-v4- | TATCAGGCCGGTAGCACACCTT (SEQ ID | E484K, N501Y, |
| F | NO: 393) | A570D |
| M6119 triple501-v4- | GTGTAATGTCAAGAATCTCAAGTGTCT | E484K, N501Y, |
| R | (SEQ ID NO: 394) | A570D |
| M6120 triple701-v1- | TGCAGGTATATGCGCTAGTTATCAGA | P681H, A701V, |
| F | (SEQ ID NO: 395) | T716H |
| M6121 triple701-v1- | GCAACAAAAGATTGCTGCATTCAGTTG | P681H, A701V, |
| R | A (SEQ ID NO: 396) | T716H |
| M6122 triple701-v2- | CAGGTATATGCGCTAGTTATCAGACTCA | P681H, A701V, |
| F | (SEQ ID NO: 397) | T716H |
| M6123 triple701-v2- | GCAACAAAAGATTGCTGCATTCAGTTG | P681H, A701V, |
| R | A (SEQ ID NO: 396) | T716H |
| M6124 triple701-v3- | GGTGCAGGTATATGCGCTAGTTATCA | P681H, A701V, |
| F | (SEQ ID NO: 398) | T716H |
| M6125 triple701-v3- | GCAACAAAAGATTGCTGCATTCAGTTG | P681H, A701V, |
| R | A (SEQ ID NO: 396) | T716H |
| M6126 triple701-v4- | ATTGGTGCAGGTATATGCGCTAGTTA | P681H, A701V, |
| F | (SEQ ID NO: 399) | T716H |
| M6127 triple701-v4- | GCAACAAAAGATTGCTGCATTCAGTTG | P681H, A701V, |
| R | A (SEQ ID NO: 396) | T716H |
| M6128 N501Y-v1-F | AGGTTTTAATTGTTACTTTCCTTTACA | N501Y |
| | (SEQ ID NO: 400) | |
| M6129 N501Y-v1-R | GCTGGTGCATGTAGAAGTTCAAAAGAA | N501Y |
| | (SEQ ID NO: 401) | |
| M6130 K417N-v1-F | TTGTAATTAGAGGTGATGAAGTCAGA | K417N |
| | (SEQ ID NO: 402) | |
| M6131 K417N-v1-R | ATTCCAAGCTATAACGCAGCCTGTAAA | K417N |
| | (SEQ ID NO: 403) | |
| M6132 K417N-v2-F | TGTAATTAGAGGTGATGAAGTCAGACA | K417N |
| | (SEQ ID NO: 404) | |
| M6133 K417N-v2-R | GAATTCCAAGCTATAACGCAGCCTGTA | K417N |
| | (SEQ ID NO: 405) | |
| M6134 Y144del-v1- | ATTGTTAATAACGCTACTAATGTTGTT | Y144del |
| F | (SEQ ID NO: 406) | |
| M6135 Y144del-v1- | CACTTTCCATCCAACTTTTGTTGTT (SEQ | Y144del |
| R | ID NO: 407) | |
| M6136 Y144del-v2- | AACGCTACTAATGTTGTTATTAAAGT | Y144del |
| F | (SEQ ID NO: 408) | |
| M6137 Y144del-v2- | ACTCTGAACTCACTTTCCATCCAACTT | Y144del |
| R | (SEQ ID NO: 409) | |
| M6138 P681H-v1-F | GGTGCAGGTATATGCGCTAGTTATCA | P681H |
| | (SEQ ID NO: 398) | |
| M6139 P681H-v1-R | AATGATGGATTGACTAGCTACACTA | P681H |
| | (SEQ ID NO: 410) | |
| M6140 P681H-v2-F | TGCAGGTATATGCGCTAGTTATCAGA | P681H |
| | (SEQ ID NO: 395) | |
| M6141 P681H-v2-R | AATGATGGATTGACTAGCTACACTA | P681H |
| | (SEQ ID NO: 410) | |
| M6142 A570D-v1-F | AGGTGTTCTTACTGAGTCTAACAAAAA | A570D |
| | (SEQ ID NO: 411) | |
| M6143 A570D-v1-R | GTCAAGAATCTCAAGTGTCTGTGGA | A570D |
| | (SEQ ID NO: 412) | |
| M6144 A570D-v2-F | AGGCACAGGTGTTCTTACTGAGTCTA | A570D |
| | (SEQ ID NO: 413) | |
| M6145 A570D-v2-R | CTCAAGTGTCTGTGGATCACGGA (SEQ | A570D |
| | ID NO: 414) | |
| M6146 double80-v1- | CAGTTTTACATTCAACTCAGGACTTGT | 69-70del, D80A |
| F | (SEQ ID NO: 415) | |
| M6147 double80-v1- | TATGTTAGACTTCTCAGTGGAAGCAAA | 69-70del, D80A |
| R | (SEQ ID NO: 416) | |
| M6148 double80-v2- | AGTTTTACATTCAACTCAGGACTTGTT | 69-70del, D80A |
| F | (SEQ ID NO: 417) | |
| M6149 double80-v2- | TGTTAGACTTCTCAGTGGAAGCAA (SEQ | 69-70del, D80A |
| R | ID NO: 418) | |
| M6150 double80-v3- | GTTTTACATTCAACTCAGGACTTGTTC | 69-70del, D80A |
| F | (SEQ ID NO: 419) | |
| M6151 double80-v3- | GTTAGACTTCTCAGTGGAAGCA (SEQ ID | 69-70del, D80A |
| R | NO: 420) | |
| M6152 double681- | GACATACCCATTGGTGCAGGTATAT | P681H, A701V |
| vl-F | (SEQ ID NO: 421) | |
| M6153 double681- | GTGGGTATGGCAATAGAGTTATTAGA | P681H, A701V |
| v1-R | (SEQ ID NO: 422) | |
| M6154 double681- | GACATACCCATTGGTGCAGGTATA (SEQ | P681H, A701V |
| v2-F | ID NO: 423) | |
| M6155 double681- | GTGGGTATGGCAATAGAGTTATTAGAG | P681H, A701V |
| v2-R | (SEQ ID NO: 424) | |
| M6156 double681- | TGACATACCCATTGGTGCAGGTA (SEQ | P681H, A701V |
| v3-F | ID NO: 425) | |
| M6157 double681- | GGGTATGGCAATAGAGTTATTAGAGTA | P681H, A701V |
| v3-R | (SEQ ID NO: 426) | |

After completion of the amplification step, the amplicon can be combined with a Cas12M08-gRNA complex, and a fluorescence-based trans-cleavage assay, as described in prior examples herein for example, is allowed to proceed. Sequences are detected using any of the gRNA sequences disclosed in Table 15. Table 15 provides exemplary guides for the UK and South African variants of the crRNA type and compatible with the Cas112M08 protein. Regarding Table 15, in the names of the guides, “d6-7” refers to deletion 60 to 70. “wt” refers to the original, wild-type SARS-CoV-2, “m” refers to a guide for a mutant variant, and “mp” refers to mutant poison. The mutant poison guides are designed to further destabilize the guides from recognizing the wild type sequence, as some guides designed to recognize the mutant may also recognize the wild type, but at a lower rate. In other words, the mutant poison guides promote stronger recognition of the mutant over the wildtype. The numbering in the “Name” column provides the amino acid position of the mutation. The Cas12M08 protein may recognize any of the following PAMs: ttcc, tcca, tttg, tHa, cttg, ccti, tHa, tttc, ttcc, tcca, ttg, tttg, ttg, tca, ctca, ttct, cttg, tttc, tcta, ctct, or ttg.

TABLE 15
Exemplary Guides for the UK and South African Variants

| Name | RNA Sequence | Target Site | Notes |
|---|---|---|---|
| d6-7-1m | UAAUUUCUACUAAGUGUAGAU | atgctgtctctgggaccaat | SARS-COV-2 UK |
| | augcugucucugggaccaau (SEQ ID | (SEQ ID NO: 483) | Variant |
| | NO: 427) | | |
| d6-7-2m | UAAUUUCUACUAAGUGUAGAU | tgctgtctctgggaccaatg | SARS-COV-2 UK |
| | ugcugucucugggaccaaug (SEQ ID | (SEQ ID NO: 484) | Variant |
| | NO: 428) | | |
| 80-1w | UAAUUUCUACUAAGUGUAGAU | ataaccctgtcctaccattt | SARS-COV-2 |
| | auaacccuguccuaccauuu (SEQ ID NO: | (SEQ ID NO: 485) | wild-type |
| | 429) | | |
| 80-1m | UAAUUUCUACUAAGUGUAGAU | Ctaaccctgtcctaccattt | SARS-COV-2 SA |
| | Cuaacccuguccuaccauuu (SEQ ID | (SEQ ID NO: 486) | Variant |
| | NO: 430) | | |
| 80-1mp | UAAUUUCUACUAAGUGUAGAU | CtaaccctAtcctaccattt | SARS-COV-2 SA |
| | CuaacccuAuccuaccauuu (SEQ ID | (SEQ ID NO: 487) | Variant |
| | NO: 431) | | |
| 80-2w | UAAUUUCUACUAAGUGUAGAU | tcaaacctcttagtaccatt | SARS-COV-2 |
| | ucaaaccucuuaguaccauu (SEQ ID NO: | (SEQ ID NO: 488) | wild-type |
| | 432) | | |
| 80-2m | UAAUUUCUACUAAGUGUAGAU | Gcaaacctcttagtaccatt | SARS-COV-2 SA |
| | Gcaaaccucuuaguaccauu (SEQ ID | (SEQ ID NO: 489) | Variant |
| | NO: 433) | | |
| 80-2mp | UAAUUUCUACUAAGUGUAGAU | GcaaacTtettagtaccatt | SARS-COV-2 SA |
| | GcaaacUucuuaguaccauu (SEQ ID | (SEQ ID NO: 490) | Variant |
| | NO: 434) | | |
| 484-1w | UAAUUUCUACUAAGUGUAGAU | taatggtgttgaaggtttta | SARS-COV-2 |
| | uaaugguguugaagguuuua (SEQ ID | (SEQ ID NO: 491) | wild-type |
| | NO: 435) | | |
| 484-1m | UAAUUUCUACUAAGUGUAGAU | taatggtgttAaaggtttta | SARS-COV-2 SA |
| | uaaugguguuAaagguuuua (SEQ ID | (SEQ ID NO: 492) | Variant |
| | NO: 436) | | |
| 484-1mp | UAAUUUCUACUAAGUGUAGAU | taatgAtgttAaaggtttta | SARS-COV-2 SA |
| | uaaugAuguuAaagguuuua (SEQ ID | (SEQ ID NO: 493) | Variant |
| | NO: 437) | | |
| 484-2w | UAAUUUCUACUAAGUGUAGAU | gtaatggtgttgaaggtttt | SARS-COV-2 |
| | guaaugguguugaagguuuu (SEQ ID | (SEQ ID NO: 494) | wild-type |
| | NO: 438) | | |
| 484-2m | UAAUUUCUACUAAGUGUAGAU | gtaatggtgttAaaggtttt | SARS-COV-2 SA |
| | guaaugguguuAaagguuuu (SEQ ID | (SEQ ID NO: 495) | Variant |
| | NO: 439) | | |
| 484-2mp | UAAUUUCUACUAAGUGUAGAU | gtaatgAtgttAaaggtttt | SARS-COV-2 SA |
| | guaaugAuguuAaagguuuu (SEQ ID | (SEQ ID NO: 496) | Variant |
| | NO: 440) | | |
| 484-3w | UAAUUUCUACUAAGUGUAGAU | aaaccttcaacaccattaca | SARS-COV-2 |
| | aaaccuucaacaccauuaca (SEQ ID NO: | (SEQ ID NO: 497) | wild-type |
| | 441) | | |
| 484-3m | UAAUUUCUACUAAGUGUAGAU | aaaccttTaacaccattaca | SARS-COV-2 SA |
| | aaaccuuUaacaccauuaca (SEQ ID NO: | (SEQ ID NO: 498) | Variant |
| | 442) | | |
| 484-3mp | UAAUUUCUACUAAGUGUAGAU | aaaccttTaacaTcattaca | SARS-COV-2 SA |
| | aaaccuuUaacaUcauuaca (SEQ ID | (SEQ ID NO: 499) | Variant |
| | NO: 443) | | |
| 501-1w | UAAUUUCUACUAAGUGUAGAU | caacccactaatggtgttgg | SARS-COV-2 |
| | caacccacuaaugguguugg (SEQ ID | (SEQ ID NO: 500) | wild-type |
| | NO: 444) | | |
| 501-1m | UAAUUUCUACUAAGUGUAGAU | caacccactTatggtgttgg | SARS-COV-2 |
| | caacccacuUaugguguugg (SEQ ID | (SEQ ID NO: 501) | UK/SA Variant |
| | NO: 445) | | |
| 501-1mp | UAAUUUCUACUAAGUGUAGAU | caacccTctTatggtgttgg | SARS-COV-2 |
| | caacccUcuUaugguguugg (SEQ ID | (SEQ ID NO: 502) | UK/SA Variant |
| | NO: 446) | | |
| 501-2w | UAAUUUCUACUAAGUGUAGAU | aacccactaatggtgttggt | SARS-COV-2 |
| | aacccacuaaugguguuggu (SEQ ID | (SEQ ID NO: 503) | wild-type |
| | NO: 447) | | |
| 501-2m | UAAUUUCUACUAAGUGUAGAU | aacccactTatggtgttggt | SARS-COV-2 |
| | aacccacuUaugguguuggu (SEQ ID | (SEQ ID NO: 504) | UK/SA Variant |
| | NO: 448) | | |
| 501-2mp | UAAUUUCUACUAAGUGUAGAU | aacccTctTatggtgttggt | SARS-COV-2 |
| | aacccUcuUaugguguuggu (SEQ ID | (SEQ ID NO: 505) | UK/SA Variant |
| | NO: 449) | | |
| 501-3w | UAAUUUCUACUAAGUGUAGAU | acccactaatggtgttggtt | SARS-COV-2 |
| | acccacuaaugguguugguu (SEQ ID | (SEQ ID NO: 506) | wild-type |
| | NO: 450) | | |
| 501-3m | UAAUUUCUACUAAGUGUAGAU | acccactTatggtgttggtt | SARS-COV-2 |
| | acccacuUaugguguugguu (SEQ ID | (SEQ ID NO: 507) | UK/SA Variant |
| | NO: 451) | | |
| 501-3mp | UAAUUUCUACUAAGUGUAGAU | acccTctTatggtgttggtt | SARS-COV-2 |
| | acccUcuUaugguguugguu (SEQ ID | (SEQ ID NO: 508) | UK/SA Variant |
| | NO: 452) | | |
| 501-4w | UAAUUUCUACUAAGUGUAGAU | gtaaccaacaccattagtgg | SARS-COV-2 |
| | guaaccaacaccauuagugg (SEQ ID NO: | (SEQ ID NO: 509) | wild-type |
| | 453) | | |
| 501-4m | UAAUUUCUACUAAGUGUAGAU | gtaaccaacaccatAagtg | SARS-COV-2 |
| | guaaccaacaccauAagugg (SEQ ID | g (SEQ ID NO: 510) | UK/SA Variant |
| | NO: 454) | | |
| 501-4mp | UAAUUUCUACUAAGUGUAGAU | gtaaccGacaccatAagtg | SARS-COV-2 |
| | guaaccGacaccauAagugg (SEQ ID | g (SEQ ID NO: 511) | UK/SA Variant |
| | NO: 455) | | |
| 570-1w | UAAUUUCUACUAAGUGUAGAU | gcagagacattgctgacact | SARS-COV-2 |
| | gcagagacauugcugacacu (SEQ ID NO: | (SEQ ID NO: 512) | wild-type |
| | 456) | | |
| 570-1m | UAAUUUCUACUAAGUGUAGAU | gcagagacattgAtgacact | SARS-COV-2 UK |
| | gcagagacauugAugacacu (SEQ ID | (SEQ ID NO: 513) | Variant |
| | NO: 457) | | |
| 570-1mp | UAAUUUCUACUAAGUGUAGAU | gcagagGcattgAtgacac | SARS-COV-2 UK |
| | gcagagGcauugAugacacu (SEQ ID | t (SEQ ID NO: 514) | Variant |
| | NO: 458) | | |
| 570-2w | UAAUUUCUACUAAGUGUAGAU | ctgacactactgatgctgtc | SARS-COV-2 |
| | cugacacuacugaugcuguc (SEQ ID | (SEQ ID NO: 515) | wild-type |
| | NO: 459) | | |
| 570-2m | UAAUUUCUACUAAGUGUAGAU | Atgacactactgatgctgtc | SARS-COV-2 UK |
| | Augacacuacugaugcuguc (SEQ ID | (SEQ ID NO: 516) | Variant |
| | NO: 460) | | |
| 570-2mp | UAAUUUCUACUAAGUGUAGAU | AtgacaTtactgatgctgtc | SARS-COV-2 UK |
| | AugacaUuacugaugcuguc (SEQ ID | (SEQ ID NO: 517) | Variant |
| | NO: 461) | | |
| 570-3w | UAAUUUCUACUAAGUGUAGAU | ccatacccacaaattttact | SARS-COV-2 |
| | guagugucagcaaugucucu (SEQ ID | (SEQ ID NO: 518) | wild-type |
| | NO: 462) | | |
| 570-3m | UAAUUUCUACUAAGUGUAGAU | ccatacccaTaaattttact | SARS-COV-2 UK |
| | guagugucaUcaaugucucu (SEQ ID | (SEQ ID NO: 519) | Variant |
| | NO: 463) | | |
| 570-3mp | UAAUUUCUACUAAGUGUAGAU | ccatacTcaTaaattttact | SARS-COV-2 UK |
| | guagugCcaUcaaugucucu (SEQ ID | (SEQ ID NO: 520) | Variant |
| | NO: 464) | | |
| 681-1w | UAAUUUCUACUAAGUGUAGAU | ccatacccacaaattttact | SARS-COV-2 |
| | ccucggcgggcacguagugu (SEQ ID | (SEQ ID NO: 518) | wild-type |
| | NO: 465) | | |
| 681-1m | UAAUUUCUACUAAGUGUAGAU | ccatacccaTaaattttact | SARS-COV-2 UK |
| | cAucggcgggcacguagugu (SEQ ID | (SEQ ID NO: 519) | Variant |
| | NO: 466) | | |
| 681-1mp | UAAUUUCUACUAAGUGUAGAU | ccatacTcaTaaattttact | SARS-COV-2 UK |
| | cAucggUgggcacguagugu (SEQ ID | (SEQ ID NO: 520) | Variant |
| | NO: 467) | | |
| 681-2w | UAAUUUCUACUAAGUGUAGAU | ccatacccacaaattttact | SARS-COV-2 |
| | gugcagaaaauucaguugcu (SEQ ID | (SEQ ID NO: 518) | wild-type |
| | NO: 468) | | |
| 681-2m | UAAUUUCUACUAAGUGUAGAU | ccatacccaTaaattttact | SARS-COV-2 UK |
| | gugUagaaaauucaguugcu (SEQ ID | (SEQ ID NO: 519) | Variant |
| | NO: 469) | | |
| 681-2mp | UAAUUUCUACUAAGUGUAGAU | ccatacTcaTaaattttact | SARS-COV-2 UK |
| | gugUagGaaauucaguugcu (SEQ ID | (SEQ ID NO: 520) | Variant |
| | NO: 470) | | |
| 701-1w | UAAUUUCUACUAAGUGUAGAU | ccatacccacaaattttact | SARS-COV-2 |
| | ugcaccaagugacauagugu (SEQ ID | (SEQ ID NO: 518) | wild-type |
| | NO: 471) | | |
| 701-1m | UAAUUUCUACUAAGUGUAGAU | ccatacccaTaaattttact | SARS-COV-2 SA |
| | uAcaccaagugacauagugu (SEQ ID | (SEQ ID NO: 521) | Variant |
| | NO: 472) | | |
| 701-1mp | UAAUUUCUACUAAGUGUAGAU | ccatacTcaTaaattttact | SARS-COV-2 SA |
| | uAcaccaaAugacauagugu (SEQ ID | (SEQ ID NO: 522) | Variant |
| | NO: 473) | | |
| 701-2w | UAAUUUCUACUAAGUGUAGAU | ccatacccacaaattttact | SARS-COV-2 |
| | uugccauacccacaaauuuu (SEQ ID NO: | (SEQ ID NO: 518) | wild-type |
| | 474) | | |
| 701-2m | UAAUUUCUACUAAGUGUAGAU | ccatacccaTaaattttact | SARS-COV-2 SA |
| | uugccauacccaUaaauuuu (SEQ ID | (SEQ ID NO: 521) | Variant |
| | NO: 475) | | |
| 701-2mp | UAAUUUCUACUAAGUGUAGAU | ccatacTcaTaaattttact | SARS-COV-2 SA |
| | uugccaCacccaUaaauuuu (SEQ ID | (SEQ ID NO: 522) | Variant |
| | NO: 476) | | |
| 716-1w | UAAUUUCUACUAAGUGUAGAU | ccatacccacaaattttact | SARS-COV-2 |
| | auugccauacccacaaauuu (SEQ ID NO: | (SEQ ID NO: 518) | wild-type |
| | 477) | | |
| 716-1m | UAAUUUCUACUAAGUGUAGAU | ccatacccaTaaattttact | SARS-COV-2 UK |
| | auugccauacccaUaaauuu (SEQ ID | (SEQ ID NO: 519) | Variant |
| | NO: 478) | | |
| 716-1mp | UAAUUUCUACUAAGUGUAGAU | ccatacTcaTaaattttact | SARS-COV-2 UK |
| | auugccGuacccaUaaauuu (SEQ ID | (SEQ ID NO: 520) | Variant |
| | NO: 479) | | |
| 716-2w | UAAUUUCUACUAAGUGUAGAU | ccatacccacaaattttact | SARS-COV-2 |
| | ccauacccacaaauuuuacu (SEQ ID NO: | (SEQ ID NO: 518) | wild-type |
| | 480) | | |
| 716-2m | UAAUUUCUACUAAGUGUAGAU | ccatacccaTaaattttact | SARS-COV-2 UK |
| | ccauacccaUaaauuuuacu (SEQ ID | (SEQ ID NO: 519) | Variant |
| | NO: 481) | | |
| 716-2mp | UAAUUUCUACUAAGUGUAGAU | ccatacTcaTaaattttact | SARS-COV-2 UK |
| | ccauacUcaUaaauuuuact (SEQ ID | (SEQ ID NO: 520) | Variant |
| | NO: 482) | | |

### Example 40

### Detection of SARS-CoV-2 with Rapid Thermocycling

This example describes the steps taken for the optimization of assay reaction conditions for rapid detection of SARS-CoV-2 with rapid thermocycling, herein named as the FASTR assay. FASTR uses an extreme PCR technique in which the speed of the PCR reaction is decreased to less than 5 minutes by near-instantaneous changes in the reaction temperature. This rapid temperature change may be accomplished by moving the reaction between heat-zones (water baths, heat blocks, etc.) of various temperatures in a thin-walled vessel, instead of cooling or heating the entire instrument for each cycle. Alternatively, the reaction volume can be pumped between two or three heat zones to achieve this rapid thermal change and drive the PCR reaction. Additional speed increases of the PCR reaction can be achieved by increasing the primer, polymerase, and Mg2+ concentrations of the reaction.

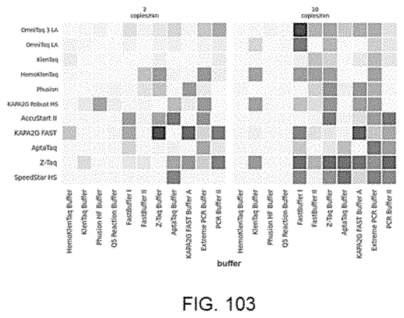

FIG. 103 depicts the results from the polymerase and buffer combinations that enabled the rapid amplification of SARS-CoV-2 using the N2 primers from the CDC assay (primer sequences presented in Table 16). The assay was performed at two target concentrations: 2 copies/reaction(rxn) and 10 copies/reaction(rxn). Reaction (rxn) conditions are as follows: initial denaturation at 98° C. for 30 seconds, followed by 45 cycles consisting of 1 second at 98° C. and 3 seconds at 65° C. Following thermocycling, amplicon was transferred to a Cas12M08 detection reaction for 30 minutes at 37° C. The data presented in FIG. 103 is the signal from the CRISPR reaction. Best performing enzyme/buffer pairs were those that gave strong signal in both tested concentrations.

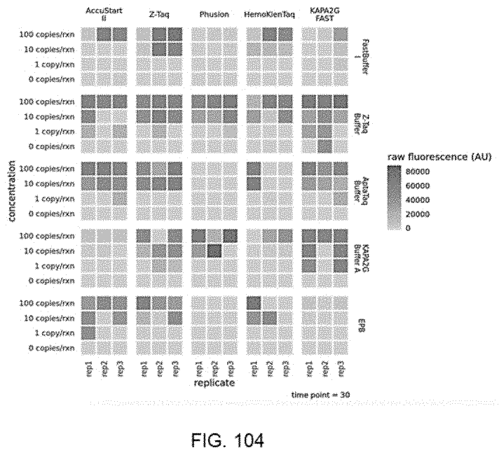

The top enzymes and buffers identified in FIG. 103 were tested at various concentrations and with multiple replicates as shown in FIG. 104 to further optimize the reaction conditions for FASTR. Reaction conditions were as follows: initial denaturation at 98° C. for 30 seconds, followed by 45 cycles consisting of 1 second at 98° C. and 3 seconds at 65° C. Primers used were from the CDC N2 assay for SARS-CoV-2 (primer sequences presented in Table 16). Following thermocycling, amplicon was transferred to a Cas12M08 detection reaction for 30 minutes at 37° C. The data presented in FIG. 104 is the signal from the CRISPR reaction. Best performing enzyme/buffer pairs were those that gave strong signal at the lowest tested concentrations and with detection across replicates.

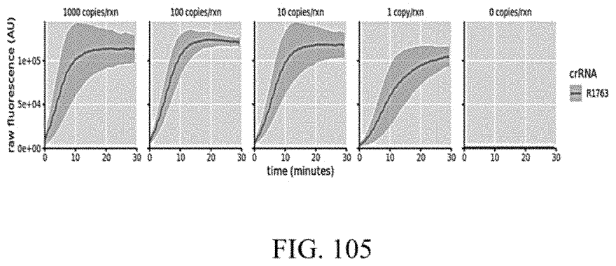

To further evaluate the performance of the FASTR assay, the limit of detection of the assay was evaluated from 1000 copies/reaction to 1 copy/reaction. Reaction conditions were as follows: reverse transcription at 55° C. for 60 seconds, initial denaturation at 98° C. for 30 seconds, followed by 45 cycles consisting of 1 second at 98° C. and 3 seconds at 65° C. Primers used were from the CDC N2 assay for SARS-CoV-2 (sequences presented in Table 16). Following thermocycling, amplicon was transferred to a Cas12M08 detection reaction for 30 minutes at 37° C. The data presented in FIG. 105 is the signal from the CRISPR reaction. The assay performed well at 1 copy/reaction and was able to detect SARS-CoV-2 at a single copy level.

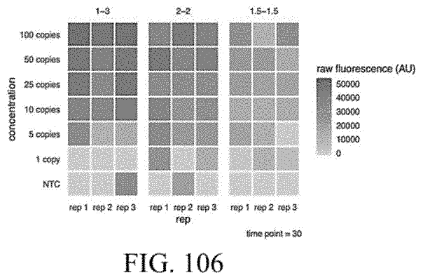

Next, the effect of variations in rapid cycling times for denaturation and annealing/extension in FASTR assay was evaluated. To determine the best cycling conditions for the FASTR assay, the performance of the assay was evaluated with varied cycling conditions. For all reactions, reverse transcription was performed at 55° C. for 60 seconds and initial denaturation at 98° C. for 30 seconds. The tested cycling conditions were: 98° C. for 1 second, 65° C. for 3 seconds; 98° C. for 2 seconds, 65° C. for 2 seconds; or 98° C. for 1.5 seconds, 65° C. for 1.5 seconds. Primers used were from the CDC N2 assay for SARS-CoV-2 (sequences presented in Table 16). Following thermocycling, amplicon was transferred to a Cas12M08 detection reaction for 30 minutes at 37° C. The results in FIG. 106 indicate that >2 seconds of annealing/extension time at 65° C. are necessary for robust sensitivity.

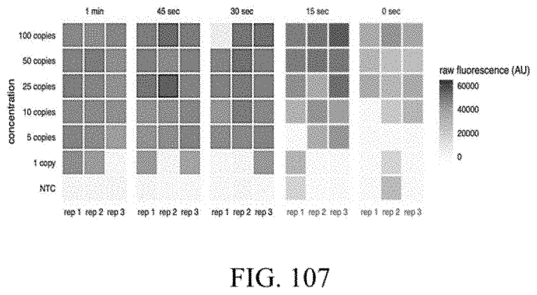

Next, in order to minimize the reverse transcription (RT) time for FASTR, the performance of the FASTR assay was evaluated with various reverse transcription incubation times at 55° C., to determine the minimal reverse transcription conditions for the FASTR assay. The results of this assay optimization in FIG. 107 indicate the assay is most robust above 30 seconds of reverse transcription.

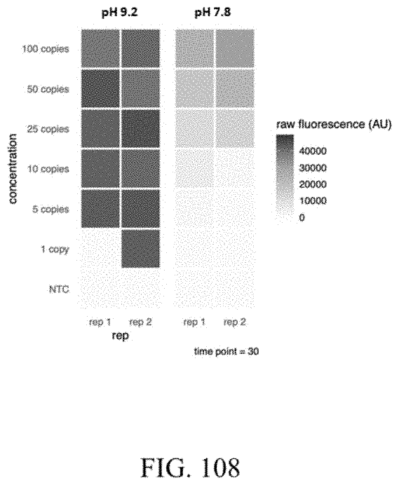

In order to test the effect of pH of the reaction buffer on the FASTR assay performance, the performance of the FASTR assay with buffers with pH of either 9.2 or pH 7.8 was evaluated. The results, as shown in FIG. 108 indicate that the higher pH buffer produced superior results in terms of amplicon yield and sensitivity.

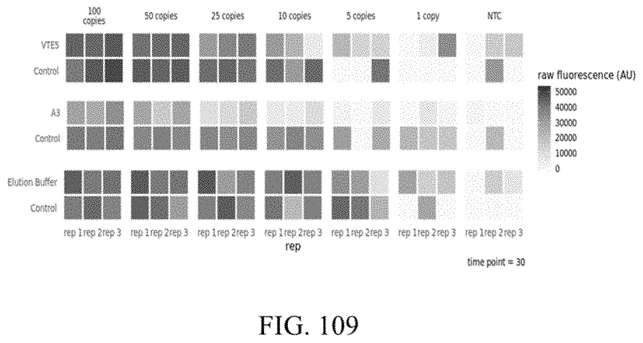

In order to test the compatibility of the FASTR assay with crude lysis buffers, the performance of the FASTR assay when combined with various crude lysis buffers was evaluated, including Crude lysis buffers VTE5, A3, and the Elution Buffer from the ChargeSwitch kit (Thermo). As seen in FIG. 109, the FASTR assay performed the best in the VTE5 lysis buffer, but performed slightly less robustly in the A3 buffer. The Elution Buffer from the ChargeSwitch kit performed similarly to the control reactions (water).

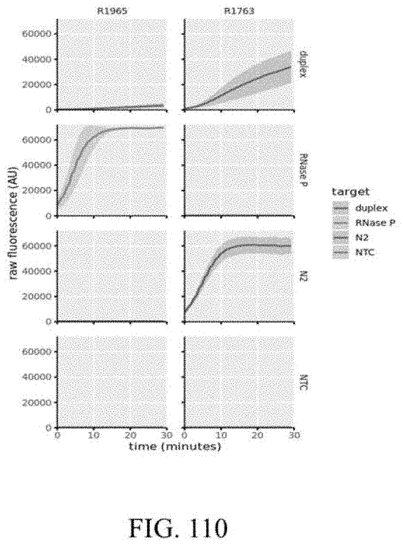

As shown in FIG. 110, initial non-optimized testing of multiplexed FASTR for SARS-CoV-2 and RNase P POP7 (endogenous control) showed that while the single-plex assays generated a robust signal in DETECTR, the duplex assay tended to generate a weak signal for SARS-CoV-2 (R1763) and almost no signal for RNase P (R1965). Reaction conditions were as follows: reverse transcription at 55° C. for 60 seconds, initial denaturation at 98° C. for 30 seconds, followed by 45 cycles consisting of 1 second at 98° C. and 3 seconds at 65° C. Primers used were from the CDC N2 assay for SARS-CoV-2, and M3637/M3638 as shown in Table 16.

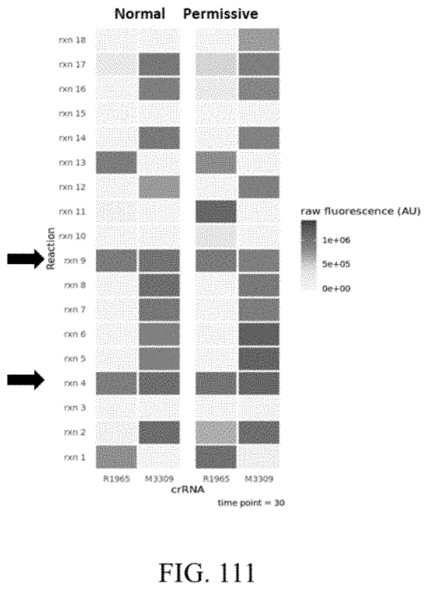

Next, considering the results of the non-optimized multiplexed FASTR assay in FIG. 110, in order to optimize multiplex FASTR for SARS-CoV-2 and RNase P, a new set of SARS-CoV-2 primers (M3257/M3258) were designed (sequences presented in Table 16). A series of experiments with varied reaction conditions containing different combinations of buffers, primer concentrations, dNTPs, and DMSO were then performed. The results of this experiment, as shown in FIG. 111, identified two reaction conditions that performed robustly for the multiplex reaction (depicted by arrows at Reaction 4 and Reaction 9). In Reaction 4, the following conditions were used: 1×FastBuffer 2, 1 μM RNase P primers, 0.5 μM CoV primers, 0.2 mM dNTPs, 2% DMSO. In Reaction 9, the following conditions were used: 1×Klentaql buffer, 1 μM RNase P primers, 0.5 μM CoV primers, 0.4 mM dNTPs, 0% DMSO. Under the normal reaction conditions, reverse transcription was performed at 55° C. for 60 seconds, initial denaturation at 98° C. for 30 seconds, followed by 45 cycles consisting of 1 second at 98° C. and 3 seconds at 65° C. Under the permissive reaction conditions, reverse transcription was performed at 55° C. for 60 seconds, initial denaturation at 98° C. for 30 seconds, followed by 45 cycles consisting of 3 seconds at 98° C. and 5 seconds at 65° C.

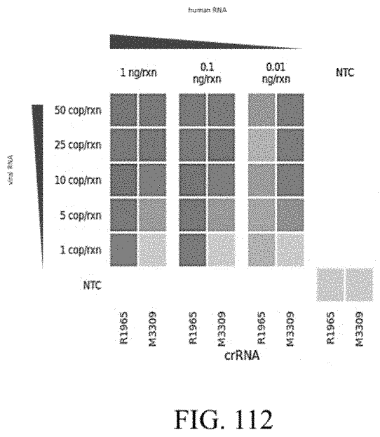

Once these conditions were optimized, the optimized multiplexed FASTR assay were evaluated at various concentrations of human RNA and viral RNA to evaluate the limit of detection of multiplex FASTR reaction. The results as shown in FIG. 111 indicate that the assay performs at a range of human RNA concentrations, while maintaining a sensitivity of ˜5 copies/reaction. Results shown in FIG. 112 are from a DETECTR reactions using either primer R1965 to detect the human RNase P, or primer R3185 (labeled M3309) to detect SARS-CoV-2. The primer sequences of R1965 and R3185 are presented in Table 16. The reaction conditions tested were as follows: reverse transcription at 55° C. for 60 seconds, initial denaturation at 98° C. for 30 seconds, followed by 45 cycles consisting of 1 second at 98° C. and 3 seconds at 65° C. Primers used were M3257/M3258 (SARS-CoV-2) and M3637/M3638 (RNase P) (presented in Table 16).

TABLE 16
Primers and gRNAs used for optimization of reaction (rxn) conditions for testing
of SARS-COV2 using rapid thermocycling

| Primer | | | |
|---|---|---|---|
| Name | Sequence | Purpose | Note |
| M2062 | TTACAAACATTGGC | PCR primer | CDC N2 assay for SARS- |
| | CGCAAA (SEQ ID | | CoV-2 |
| | NO: 192) | | |
| M2063 | GCGCGACATTCCG | PCR primer | CDC N2 assay for SARS- |
| | AAGAA (SEQ ID NO: | | CoV-2 |
| | 193) | | |
| R1763 | UAAUUUCUACUAA | Cas12 gRNA | Compatible with M2062 / |
| | GUGUAGAUCCCCC | | M2063 |
| | AGCGCUUCAGCGU | | |
| | UC (SEQ ID NO: 171) | | |
| M3637 | CCTCCGTGATATGG | PCR primer | Human RNase P POP7 |
| | CTCTTC (SEQ ID NO: | | |
| | 523) | | |
| M3638 | AGAGTCCTTTGGGC | PCR primer | Human RNase P POP7 |
| | TTCC (SEQ ID NO: | | |
| | 524) | | |
| R1965 | UAAUUUCUACUAA | Cas12 gRNA | Compatible with M3637 / |
| | GUGUAGAUUUACA | | M3638 |
| | UGGCUCUGGUCCG | | |
| | AG (SEQ ID NO: 218) | | |
| M3257 | AGGTGCCTGGAAT | PCR primer | SARS-COV-2, orf1ab |
| | ATTGGTGAACAG | | |
| | (SEQ ID NO: 525) | | |
| M3258 | TCAAGAGTGCGGG | PCR primer | SARS-COV-2, orf1ab |
| | AGAAAATTGATCG | | |
| | (SEQ ID NO: 526) | | |
| R3185 | UAAUUUCUACUAA | Cas12 gRNA | Compatible with M3257, |
| | GUGUAGAUCAUCA | | M3258 |
| | GAGGCUGCUCGUG | | |
| | UU (SEQ ID NO: 527) | | |

### Example 41

### Design of a Guide Screen for Detecting SNP Variants of SARS-CoV-2

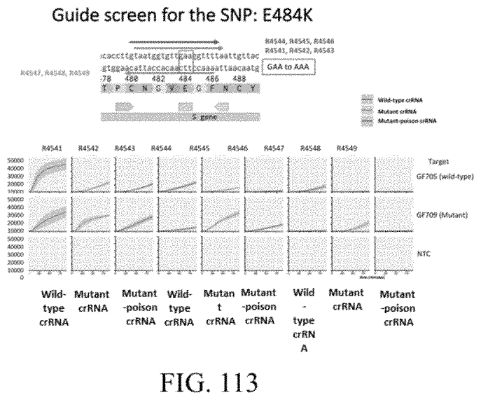

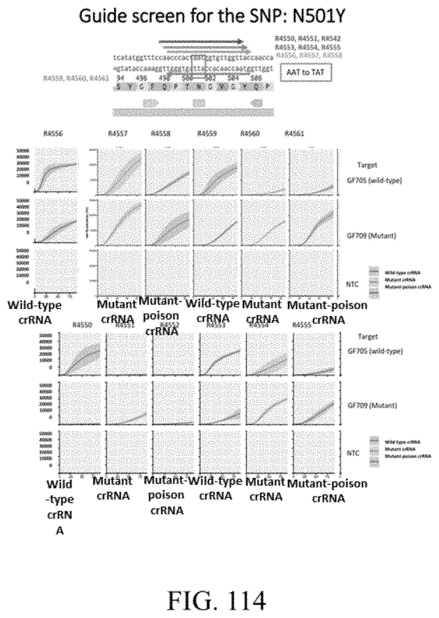

This example describes a guide screen capable of identifying guide RNAs that can enable detection of different SNP locations within the spike region of SARS-CoV-2. A series of guides were designed to differentiate between the wild-type and mutant sequences at E484K and N501Y. The E484K SNP is representative of the B.1.351 variant of SARS-CoV-2 found in South Africa. The N501Y SNP is characteristic of the B.1.1.7 variant of SARS-CoV-2 found in the U.K. The N501Y is also found in the B.1.351 variant. The first step to identifying appropriate cfRNA guides for SNP detection was to screen all the guides against gene fragments of a wild-type sequence and a mutant sequence. Gene fragments composed of wild-type or mutant sequence were amplified and used as templates in DETECTR reactions as shown in FIG. 113. Gene fragments composed of wild-type or mutant sequence (E484K) were screened for guide sequences that could distinguish between them. In a second similar assay, as shown in FIG. 114, gene fragments composed of wild-type or mutant sequence (N501Y) were screened for guide sequences that could distinguish between them.

The data shown in FIG. 113 and FIG. 114 are examples of guide screens designed to identify two different SNP locations (E484K and N501Y respectively). As shown in FIG. 114, the ideal guides recognize their specific target down to the nucleotide sequence (e.g. R4550). As shown in FIG. 113, less ‘stringent’ guides may not differentiate the single nucleotide change between wild-type and mutant sequence (e.g. 4541). As shown in FIG. 113, guides often show a stronger preference for one over the other sequences, which, depending on the ‘stringency’ will provide sufficient SNP differentiation. An example of this is R4542 or R4545 both of which should identify the mutant SNP E484K, but show low levels of wild-type detection (FIG. 113). The timing and strength of the guides in the wild-type reaction is such that these guides still provide strong differentiation of the mutant sequence.

1. A method of assaying for a segment of a coronavirus target nucleic acid in a sample, the method comprising:
a) contacting the sample to:
i) a detector nucleic acid; and
ii) a composition comprising a programmable nuclease and a non-naturally occurring guide nucleic acid that hybridizes to a segment of the target nucleic acid, wherein the programmable nuclease cleaves the detector nucleic acid upon hybridization of the non-naturally occurring guide nucleic acid to the segment of the coronavirus target nucleic acid; and
b) assaying for a change in a signal, wherein the change in the signal is produced by cleavage of the detector nucleic acid.

2. The method of claim 1, wherein the coronavirus target nucleic acid is from SARS-CoV-2.

3. The method of claim 1, wherein the coronavirus target nucleic acid is from an E gene, an N gene, or a combination thereof.

4. The method of claim 1, wherein:
(a) the coronavirus target nucleic acid has a sequence of any one of SEQ ID NO: 179-SEQ ID NO: 184;
(b) the guide nucleic acid is a guide RNA;
(c) the guide nucleic acid has at least 80%, at least 85%, at least 90%, at least 92%, at least 95%, at least 97%, or at least 99% sequence identity to any one of SEQ ID NO: 171-SEQ ID NO: 178, SEQ ID NO: 218, or SEQ ID NO: 219; or
(d) the guide nucleic acid is selected from any one of SEQ ID NO: 171-SEQ ID NO: 178, SEQ ID NO: 218, or SEQ ID NO: 219.

5.-7. (canceled)

8. The method of claim 1, further comprising amplifying the coronavirus target nucleic acid, optionally wherein: (a) the amplifying comprises thermal cycling amplification; (b) the amplifying comprises isothermal amplification; (c) the amplifying comprises loop mediated amplification (LAMP); or (d) the amplifying comprises contacting the sample to amplification primers selected from SEQ ID NO: 194-SEQ ID NO: 199 or SEQ ID NO: 202-SEQ ID NO: 205.

9.-18. (canceled)

19. The method of claim 1, wherein the method further comprises reverse transcribing the coronavirus target nucleic acid or the segment thereof.

20.-23. (canceled)

24. The method of claim 1, the method further comprising assaying for a control sequence by contacting a control nucleic acid to a second detector nucleic acid and a composition comprising the programmable nuclease and a non-naturally occurring guide nucleic acid that hybridizes to a segment of the control nucleic acid, wherein the programmable nuclease cleaves the detector nucleic acid upon hybridization of the non-naturally occurring guide nucleic acid to the segment of the control nucleic acid.

25. The method of claim 24, wherein: (a) the control nucleic acid is RNase P; or (b) the control nucleic acid has a sequence of SEQ ID NO: 220.

26. (canceled)

27. The method of claim 1, wherein: (a) the guide nucleic acid has at least 80%, at least 85%, at least 90%, at least 92%, at least 95%, at least 97%, or at least 99% sequence identity to SEQ ID NO: 178, SEQ ID NO: 218, or SEQ ID NO: 219; or (b) or the guide nucleic acid comprises the sequence of SEQ ID NO: 178, SEQ ID NO: 218, or SEQ ID NO: 219.

28. (canceled)

29. The method of claim 1, wherein the method comprises one or more steps carried out on a lateral flow strip.

30. The method of claim 29, wherein (a) the lateral flow strip comprises a sample pad region, a control line, and a test line; (b) presence or absence of an uncleaved reporter molecule is detected at the control line; and (c) presence or absence of a cleaved reporter molecule is detected at the test line.

31.-32. (canceled)

33. The method of claim 1, wherein the method comprises one or more steps carried out in a microfluidic cartridge.

34.-35. (canceled)

36. The method of claim 1, wherein:
(a) the programmable nuclease comprises an RuvC catalytic domain;
(b) the programmable nuclease is a type V CRISPR/Cas effector protein;
(c) the programmable nuclease is a Cas12 protein;
(d) the programmable nuclease comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, or a C2c9 polypeptide;
(e) the programmable nuclease has at least 80%, at least 85%, at least 90%, at least 92%, at least 95%, at least 97%, or at least 99% sequence identity to any one of SEQ ID NO: 18-SEQ ID NO: 60;
(f) the programmable nuclease comprises a sequence selected from SEQ ID NO: 18-SEQ ID NO: 60;
(g) the programmable nuclease is a Cas14 protein;
(h) the programmable nuclease comprises a Cas14a polypeptide, a Cas14b polypeptide, a Cas14c polypeptide, a Cas14d polypeptide, a Cas14e polypeptide, a Cas14f polypeptide, a Cas14g polypeptide, a Cas14h polypeptide, a Cas14i polypeptide, a Cas14j polypeptide, or a Cas14k polypeptide;
(i) the programmable nuclease has at least 80%, at least 85%, at least 90%, at least 92%, at least 95%, at least 97%, or at least 99% sequence identity to any one of SEQ ID NO: 61-SEQ ID NO: 152;
(j) the programmable nuclease comprises a sequence selected from SEQ ID NO: 61-SEQ ID NO: 152;
(k) the programmable nuclease is a CasΦ protein;
(l) the programmable nuclease has at least 80%, at least 85%, at least 90%, at least 92%, at least 95%, at least 97%, or at least 99% sequence identity to any one of SEQ ID NO: 221-SEQ ID NO: 268; or
(m) the programmable nuclease comprises a sequence selected from SEQ ID NO: 221-SEQ ID NO: 268.

37.-48. (canceled)

49. The method of claim 1, further comprising in vitro transcribing amplified coronavirus target nucleic acid.

50.-52. (canceled)

53. The method of claim 1, wherein:
(a) the programmable nuclease is a type VI CRISPR/Cas effector protein;
(b) the programmable nuclease is a Cas13 protein;
(c) the programmable nuclease comprises a Cas13a polypeptide, a Cas13b polypeptide, a Cas13c polypeptide, a Cas13c polypeptide, a Cas13d polypeptide, or a Cas13e polypeptide;
(d) the programmable nuclease has at least 80%, at least 85%, at least 90%, at least 92%, at least 95%, at least 97%, or at least 99% sequence identity to any one of SEQ ID NO: 153-SEQ ID NO: 170;
(e) the programmable nuclease comprises a sequence selected from SEQ ID NO: 153-SEQ ID NO: 170.

54.-57. (canceled)

58. The method of claim 1, further comprising multiplexed detection of more than one coronavirus target nucleic acid, and optionally a control nucleic acid.

59.-62. (canceled)

63. A composition comprising a non-naturally occurring guide nucleic acid having at least 80%, at least 85%, at least 90%, at least 92%, at least 95%, at least 97%, or at least 99% sequence identity to any one of SEQ ID NO: 171-SEQ ID NO: 177.

64. The composition of claim 63, wherein the guide nucleic acid is selected from any one of SEQ ID NO: 171-SEQ ID NO: 177.

65. The composition of claim 63, further comprising: a detector nucleic acid, a programmable nuclease, reagents for amplification, reagents for reverse transcription, reagents for in vitro transcription, a lysis buffer, a control nucleic acid, and/or a guide nucleic acid.

66.-74. (canceled)

75. A device that comprises:
a sample interface configured to receive a sample that comprises a coronavirus sequence of interest;
a channel in fluid communication with the sample interface and a detection chamber, said channel comprising one or more movable mechanisms to separate the sample into a plurality of droplets, wherein said detection chamber is configured to receive and contact the plurality of droplets with at least one programmable nuclease probe disposed on a surface of said detection chamber, wherein said at least one programmable nuclease probe comprises a guide nucleic acid complexed with a programmable nuclease; and
a plurality of sensors that determine a presence of said coronavirus sequence of interest by detecting a signal produced upon cleavage of a target nucleic acid region of said at least one sequence of interest by said at least one programmable nuclease probe.
